(19) United States
(12) Patent Application Publication
(10) Pub. No.: US 2026/0365682 A1
(43) Pub. Date: Nov. 19, 2026

(54) MODULATORS OF TNF-ALPHA ACTIVITY

(71) Applicant: Forward Therapeutics, Inc., Palm Beach Gardens, FL (US)

(72) Inventor: Toufike KANOUNI, Palm Beach Gardens, FL (US)

(21) Appl. No.: 19/137,383

(22) PCT Filed: Dec. 12, 2023
(86) PCT No.: PCT/US2023/083676

§ 371 Date: Jun. 10, 2025

Related U.S. Application Data
(60) Provisional application No. 63/387,231, filed on Dec. 13, 2022.

(51) Int. Cl. C07F 9/6561; A61K 31/55; A61K 31/675; C07B 59/00; C07D 487/18; C07D 487/22

(52) U.S. Cl. CPC C07F 9/6561; A61K 31/55; A61K 31/675; C07B 59/002; C07B 59/004; C07D 487/18; C07D 487/22; C07B 2200/05

(57) ABSTRACT

Provided herein are inhibitors of TNF-α, pharmaceutical compositions comprising the inhibitory compounds, and methods for using the TNF-α inhibitory compounds for the treatment of diseases or disorders.

### CROSS-REFERENCE TO RELATED APPLICATION

This application claims the benefit of U.S. Provisional Application No. 63/387,231, filed Dec. 13, 2022, which is incorporated herein by reference in its entirety.

### BACKGROUND

Tumor necrosis factor alpha (TNF-α) is an inflammatory cytokine that is responsible for a wide range of signaling events within cells. Aberrant TNF-α signaling gives rise to inflammatory conditions and is thought to be an important component of inflammatory disease, such as rheumatoid arthritis.

### BRIEF SUMMARY OF THE INVENTION

Provided herein are inhibitors of TNF-α, pharmaceutical compositions comprising said inhibitory compounds, and methods for using said inhibitory compounds for the treatment of inflammatory or autoimmune disease or disorder.

One embodiment provides a compound of Formula (I), or pharmaceutically acceptable salt, solvate, N-oxide, or deuteroisotope thereof:

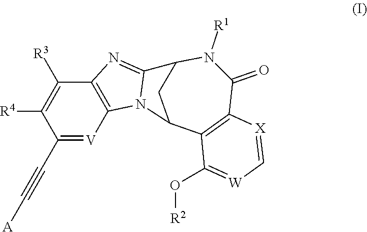

wherein,
Group A is selected from H, —CO₂R⁶, —CON(R⁶)₂, optionally substituted C1-C6 alkyl, optionally substituted C1-C6 alkyl further substituted with —O-(optionally substituted heterocyclyl), optionally substituted C3-C6 carbocyclyl, optionally substituted C4-C7 carbocyclylalkyl, optionally substituted heterocyclyl, optionally substituted heterocyclyl further substituted with an optionally substituted carbocyclyl or heterocyclyl, optionally substituted heterocyclylalkyl, optionally substituted heteroaryl, optionally substituted heteroaralkyl, optionally substituted aryl, optionally substituted aralkyl;
V is N or C—R⁵;
W is N or C—H;
X is N or C—H;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C3 alkyl;
R³ is hydrogen or halogen;
R⁴ is hydrogen, halogen, or optionally substituted C1-C6 alkyl;
R⁵ is hydrogen, halogen, or optionally substituted C1-C6 alkyl; and
R⁶ is hydrogen or optionally substituted C1-C6 alkyl.

One embodiment provides a compound of Formula (II), or pharmaceutically acceptable salt, solvate, N-oxide, or deuteroisotope thereof:

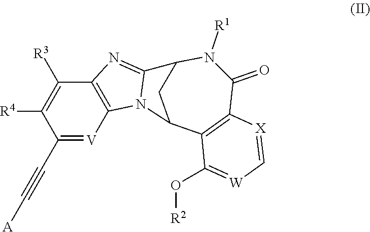

wherein,
Group A is optionally substituted C1-C6 alkyl, optionally substituted C3-C6 carbocyclyl, optionally substituted 5- or 6-membered heterocyclyl, optionally substituted 5- or 6-membered heteroaryl, or optionally substituted phenyl;
V is N or C—R;
W is N or C—H;
X is N or C—H;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C3 alkyl;
R³ is hydrogen or halogen;
R⁴ is hydrogen, halogen, or optionally substituted C1-C6 alkyl; and
R⁵ is hydrogen, halogen, or optionally substituted C1-C6 alkyl.

One embodiment provides a pharmaceutical composition comprising a compound of Formula (I) or (II), or pharmaceutically acceptable salt, solvate, N-oxide, or deuteroisotope thereof, and at least one pharmaceutically acceptable excipient.

One embodiment provides a method of treating a disease or disorder in a patient in need thereof comprising administering to the patient a compound of Formula (I) or (II), or pharmaceutically acceptable salt, solvate, N-oxide, or deuteroisotope thereof. Another embodiment provides the method wherein the disease or disorder is rheumatoid arthritis.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)R^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})ₜS(O)R^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene).

Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})ₜS(O)R^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})ₜS(O)R^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})ₜS(O)R^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, cyano, methoxy, or trifluoromethyl), fluoroalkyl, carbocyclyl (optionally substituted with halogen, hydroxy, cyano, methoxy, or trifluoromethyl), carbocyclylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R—O—R^{c}—C(O)N(R^{a})₂, —R^{b}—N(R^{a})C(O)OR^{a}, —R^{b}—N(R^{a})C(O)R^{a}, —R^{b}—N(R^{a})ₜS(O)R^{a} (where t is 1 or 2), —R^{b}—S(O)ₜR^{a} (where t is 1 or 2), —R^{b}—S(O)ₜOR^{a} (where t is 1 or 2), —R^{b}—S(O)ₜN(R^{a})₂ (where t is 1 or 2), and —P(O)(R^{a})₂ where each R^{a} is independently hydrogen, alkyl (optionally substituted with halogen, hydroxy, cyano, methoxy, or trifluoromethyl), fluoroalkyl, cycloalkyl (optionally substituted with halogen, hydroxy, cyano, methoxy, or trifluoromethyl), cycloalkylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R—OR^{a}, —R^{b}—OC(O)—R^{a}, —R^{b}—OC(O)—OR^{a}, —R—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) —Rb—S(O)tN(Ra)2 (where t is 1 or 2), and —P(O)(R^{a})₂, where each R^{a} is independently hydrogen, alkyl (optionally substituted with halogen, hydroxy, cyano, methoxy, or trifluoromethyl), fluoroalkyl, cycloalkyl (optionally substituted with halogen, hydroxy, cyano, methoxy, or trifluoromethyl), cycloalkylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R—OR^{a}, —R^{b}—OC(O)—R^{a}, —R^{b}—OC(O)—OR^{a}, —R—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) —Rb—S(O)tN(Ra)2 (where t is 1 or 2), and —P(O)(R^{a})₂, where each R^{a} is independently hydrogen, alkyl (optionally substituted with halogen, hydroxy, cyano, methoxy, or trifluoromethyl), fluoroalkyl, cycloalkyl (optionally substituted with halogen, hydroxy, cyano, methoxy, or trifluoromethyl), cycloalkylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) —Rb—S(O)tN(Ra)2 (where t is 1 or 2), and —P(O)(R^{a})₂, where each R^{a} is independently hydrogen, alkyl (optionally substituted with halogen, hydroxy, cyano, methoxy, or trifluoromethyl), fluoroalkyl, cycloalkyl (optionally substituted with halogen, hydroxy, cyano, methoxy, or trifluoromethyl), cycloalkylalkyl (optionally substituted with halogen, hydroxy, cyano, methoxy, or trifluoromethyl), aryl (optionally substituted with halogen, hydroxy, cyano, methoxy, or trifluoromethyl), aralkyl (optionally substituted with halogen, hydroxy, cyano, methoxy, or trifluoromethyl), heterocyclyl (optionally substituted with halogen, hydroxy, cyano, methoxy, or trifluoromethyl), heterocyclylalkyl (optionally substituted with halogen, hydroxy, cyano, methoxy, or trifluoromethyl), heteroaryl (optionally substituted with halogen, hydroxy, cyano, methoxy, or trifluoromethyl), or heteroarylalkyl (optionally substituted with halogen, hydroxy, cyano, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

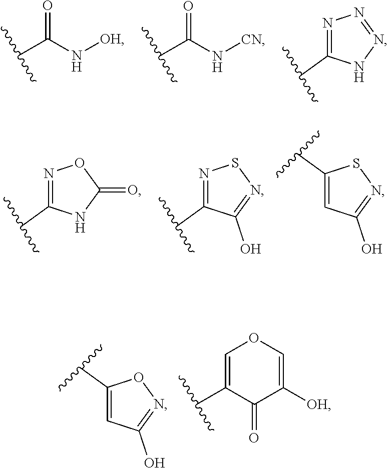

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

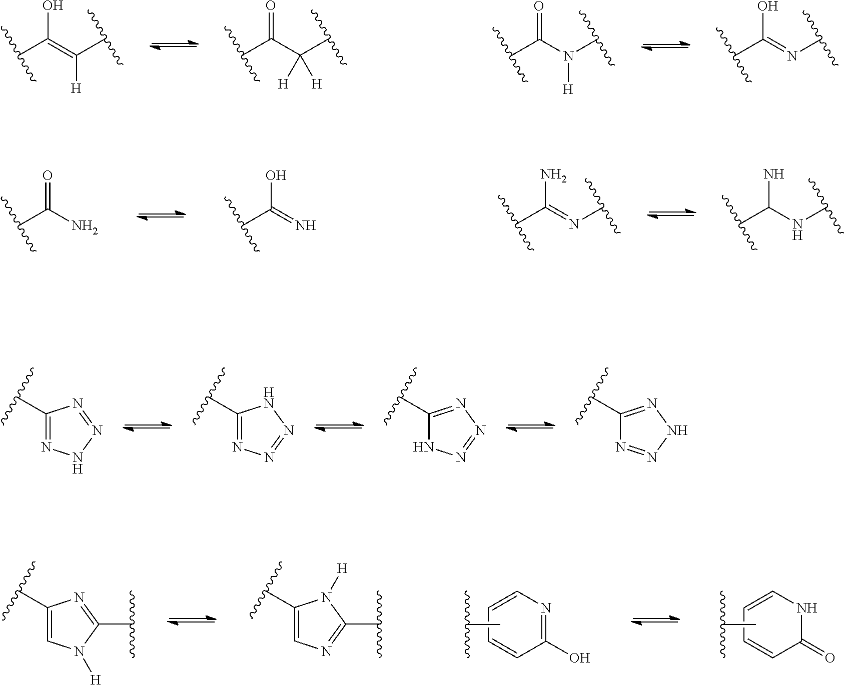

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. Said compounds with ²H replacement are described herein as deuteroisotopes. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

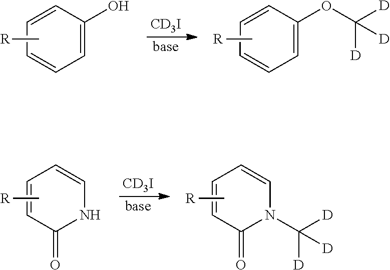

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

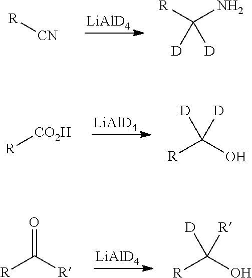

Deuterium gas and palladium catalyst are employed to reduce unsaturated carbon-carbon linkages and to perform a reductive substitution of aryl carbon-halogen bonds as illustrated, by way of example only, in the reaction schemes below.

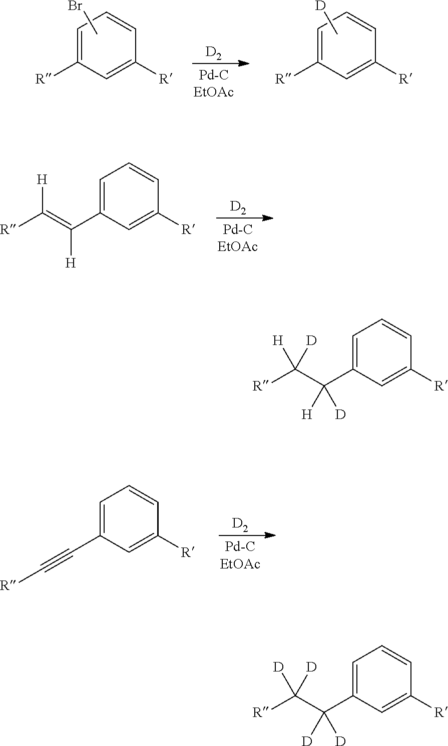

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the TNF-α inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Tumor Necrosis Factor Alpha (TNFα) Protein and Function

Tumor necrosis factor alpha (TNFα) proteins are members of the TNF superfamily, comprising various transmembrane proteins with a homologous TNF domain forming trimers. The TNF superfamily comprises 19 family members, including, but not limited to tumor necrosis factor alpha (also known as tumor necrosis factor, or TNF), lymphotoxin alpha (TNFβ), lymphotoxin beta (TNFγ), OX40 ligand, CD40 ligand, Fas ligand, CD27 ligand, CD30 ligand, CD137 ligand, CD137 ligand, and TNF-related apoptosis-inducing ligand. TNFα proteins are cytokines and adipokines (cytokines secreted by adipose tissue).

TNFα is a transmembrane protein, with soluble TNFα (sTNFα) released via protein cleavage. The sTNFα can propagate signaling by binding to two receptors, TNFR1 and TNFR2. TNFα is a regulator of immune responses for cell signaling and can mediate cell survival and cell death inducing signaling. There are two receptors for TNF signaling, TNFR1 and TNFR2. sTNFα-TNFR1 signaling promotes immune cell activation and drives acute and chronic inflammation. Membrane TNFα-TNFR2 signaling promotes inflammation resolution, immune cell regulatory functions and cell survival.

The extracellular region of both TNFR1 and TNFR2 have four homologous cysteine-rich domains, but they have structurally different intracellular regions. TNFR1 has a protein binding region called a death domain which allows homo- and hetero-typic interactions with other death domain-containing proteins. In contrast, TNFR2 has a TNF Receptor Associated Factor TRAF) that interacts with TRAF family of signaling adaptors. The distinct profiles and differences of the two TNF receptors influence the cellular activity and physiological roles. TNFR1 can activate NF-κB and MAPK signaling, and cell death, and is important to regulate for inflammatory diseases. TNFR2 is highly regulated and restricted to specific cell types such as endothelial cells and T cells. TNFR1 primarily promotes tissue degeneration and inflammation and TNFR2 typically mediates local homeostatic effects such as tissue regeneration and cell survival (D. Fresegna et al., Cells, 2020, 9, 2290).

Binding of TNFα to TNFR1 can activate NF-κB for mediating transcription of various proteins involved in cell survival and proliferation, anti-apoptotic factors, and inflammatory response. Further, the MAPK pathway can also be activated by binding of TNFα to TNFR1, which is involved in cell differentiation and proliferation. When TNF binds to TNFR1, it triggers receptor trimerization, leading to the assembly of a TNFR1-associated signaling complex. This complex recruits the receptor interacting protein 1 (RIP2) and TNF receptor associated death domain (TRADD) to the TNFR1 through the receptive death domains. TRADD then recruits adaptor proteins TRAF2 and TRAF5, which can engage the E3 ligases cellular inhibitors of apoptosis (c-IAP1, c-IAP2). C-IAP1/2 are important for TNFR1 complex signaling, which can eventually lead to the recruitment of the signaling kinase complexes of kinase IKKα and IKKβ, which are inhibitors of kappa B kinase 1 and 2, and transforming growth factor beta-activated kinase 1 (TAK1) leading to activation of NF-κB and MAPK signaling. Activation of these signaling pathways can result in gene activation and expression of pro-inflammatory cytokines and pro-survival proteins.

TNF signaling is regulated by post-translational ubiquitination, which is essential for my biological processes. Post-translational modifications of TNFR1-associated signaling complexes can result in a change from inflammatory gene signaling to cell death. This switch is dependent upon the ubiquitination status of RIP1, which is formed as part of the TNFR1-associated signaling complex from TNFa binding.

TNF has long been known to be a key regulator of the inflammatory response, and recently has been known to be involved n brain functioning (D. Fresegna et al., Cells, 2020, 9, 2290). As a regulator of the inflammatory response, TNF can regulate many aspects of T cell biology including, but not limited to proliferation, survival, priming, and apoptotic fate. TNF is also known to play a role in conclusion of lymphocyte response, by the ability to promote cell death in both CD4 and CD8P T cells, through TNFR1. Specific inflammatory conditions can also result in TNFR2 promoting or supporting T cell apoptosis.

In normal adult brains, TNF is expressed at low levels, and it is believed that the expression could be influenced by presence or absence of cytokines that can cross the blood brain barrier. TNFRs in the brain are expressed by glia and neurons cells, and have regulatory functions, including, but not limited to homeostatic synaptic plasticity, astrocyte-mediated synaptic transmission, and neurogenesis. These functions are useful for regulating learning and memory functions amongst other roles.

TNF is recognized to be physiological gliotransmitter for the communication between neurons and glial cells, which in turn affects synaptic regulation. Glial TNF is important for maintenance of normal surface expression of AMPA receptors, and for homeostatic synaptic scaling, which allows for adjustment of the strength of all synapses on a neuron.

### Prior Art Small Molecules Inhibitors

Diseases treated with biologic TNFα inhibitors include, but are not limited to rheumatoid arthritis, inflammatory bowel disease, psoriatic arthritis, psoriasis, and ankylosing spondylitis. Patients with neuroinflammatory conditions and degenerative disease, including, but not limited to Alzheimer's disease, Parkinson's disease, multiple sclerosis, treatment resistant depression, and tinnitus, may benefit from treatment with oral CNS sTNFα inhibitors by disrupting the sTNFα signaling and sparing the mTNFα signaling. Previous reports have also indicated targeting TNFR2 for treating Alzheimer's Disease (N. Orti-Casañ et al., Front Neurosci. 2019; 13: 49).

Small molecules have been developed for treatment of rheumatoid arthritis as some patients have responded poorly to monotherapy of approved anti-TNFα drugs (J. D. Dietrich et al., J. Med. Chem. 2021, 64, 417-429). Anti-TNFα drugs have also been expanded for use in other chronic autoimmune diseases, including, but not limited to, Crohn's disease, psoriasis, psoriatic arthritis, ulcerative colitis inflammatory bowel disease, ankylosing spondylitis, and juvenile rheumatoid arthritis. Small molecules have been developed as an alternative to anti-TNFa biologics since the long-term clinical response rate is generally around 60-70% for rheumatoid arthritis.

Previous research has also indicated that TNFα inhibitors can be therapeutic for treatment of multiple sclerosis (D. Fresegna et al., Cells, 2020, 9, 2290). There has been evidence of the involvement of TNF in various pathological issues of multiple sclerosis, including immune dysregulation, demyelination, synaptopathy, and neuroinflammation. TNFa inhibitors have the potential for treatment of multiple sclerosis, other potential chronic neurodegenerative diseases of the central nervous system.

More than 50 million Americans struggle with tinnitus, which is the hearing of a sound with no external source. It has been shown that TNFα is necessary for noise-induced neuroinflammation and synaptic imbalance (W. Wang et al., PLoS Biol. 2019 Jun. 18; 17(6):e3000307; A. Shulman et al., Curr Top Behav Neurosci. 2021; 51:161-174). It is believed that certain inhibitors of TNFa have activities for treating tinnitus.

Recent reports also indicate that TNFα inhibitors can be used alone or in combination for treatment with inflammatory bowel disease (S. F. Fowler Braga and K. J. Clark, US Pharm. 2021; 46(5):34-37). TNFα is a mediator of the abnormal immune response of inflammatory bowel disease, which leads to disruption of the intestinal mucosa and epithelial wall barrier. The anti-TNF agents can block TNF-mediated activation of the proinflammatory pathways to result in decreased immune-mediated inflammation.

Small molecule sTNF α inhibitors are active in pharmacology models of sTNFα/TNFR1 signaling in addition to demonstrating efficacy in a model of collagen antibody induced arthritis. There is currently limited data in the public domain for small molecule sTNF α inhibitors. Some TNFα inhibitors include, but are not limited to XPro1595, Etanercept, Infliximab, Adalimumab, Certolizumab pegol, Golimumab, and other inhibitors described in “TNF-α: The Shape of Small Molecules to Come?” (A. Dömling and X. Li, Drug Discov Today 2022 January; 27(1):3-7) and “Small Molecules that Inhibit TNF Signalling by Stabilising an Asymmetric Form of the Trimer (J. O'Connell et al., Nature Communications 10, 5795 (2019)). Additional small molecule inhibitors of TNFa include, but are not limited to the inhibitors described in “Biologic-like In Vivo Efficacy with Small Molecule Inhibitors of TNFα Identified Using Scaffold Hopping and Structure-Based Drug Design Approaches” (H-Y Xiao et al., J. Med. Chem. 2020, 15050-15071), “Development of Orally Efficacious Allosteric Inhibitors of TNFa via Fragment-Based Drug Design” (J. D. Dietrich et al., J. Med. Chem. 2021, 64, 417-429), and “Small-Molecule Inhibition of TNF-α” (M. M. He et al., Science, 310 (2015), 1022-1025).

Small molecule sTNFα inhibitors have potential as a valuable therapy for patients currently treated with biologic TNFα inhibitors which affect mTNFα with the ability to fine tune oral dosing requirements and avoid anti-drug antibody responses, thereby improving short and long responses (A. Dömling and X. Li, Drug Discov Today 2022 January; 27(1):3-7).

### Novel Compounds Inhibiting TNF-α

In one aspect, provided herein are TNF-α inhibitory compounds.

One embodiment provides a compound of Formula (I), or pharmaceutically acceptable salt, solvate, N-oxide, or deuteroisotope thereof:

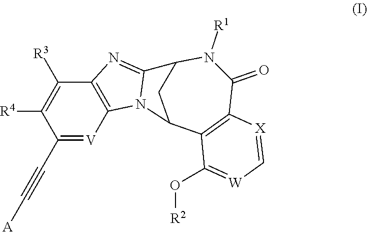

wherein,
Group A is selected from H, —CO₂R⁶, —CON(R⁶)₂, optionally substituted C1-C6 alkyl, optionally substituted C1-C6 alkyl further substituted with —O-(optionally substituted heterocyclyl), optionally substituted C3-C6 carbocyclyl, optionally substituted C4-C7 carbocyclylalkyl, optionally substituted heterocyclyl, optionally substituted heterocyclyl further substituted with an optionally substituted carbocyclyl or heterocyclyl, optionally substituted heterocyclylalkyl, optionally substituted heteroaryl, optionally substituted heteroaralkyl, optionally substituted aryl, optionally substituted aralkyl;
V is N or C—R⁵;
W is N or C—H;
X is N or C—H;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C3 alkyl;
R³ is hydrogen or halogen;
R⁴ is hydrogen, halogen, or optionally substituted C1-C6 alkyl;
R⁵ is hydrogen, halogen, or optionally substituted C1-C6 alkyl; and
R⁶ is hydrogen or optionally substituted C1-C6 alkyl.

One embodiment provides a compound of Formula (II), or pharmaceutically acceptable salt, solvate, N-oxide, or deuteroisotope thereof:

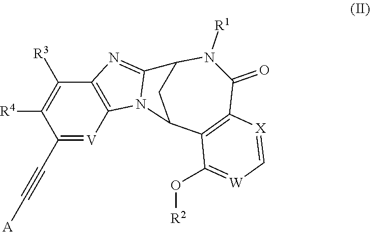

wherein,
Group A is optionally substituted C1-C6 alkyl, optionally substituted C3-C6 carbocyclyl, optionally substituted 5- or 6-membered heterocyclyl, optionally substituted 5- or 6-membered heteroaryl, or optionally substituted phenyl;
V is N or C—R⁵;
W is N or C—H;
X is N or C—H;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C3 alkyl;
R³ is hydrogen or halogen;
R⁴ is hydrogen, halogen, or optionally substituted C1-C6 alkyl; and
R⁵ is hydrogen, halogen, or optionally substituted C1-C6 alkyl.

One embodiment provides the compound of Formula (I) or (II), or pharmaceutically acceptable salt, solvate, N-oxide, or deuteroisotope thereof, wherein R¹ is hydrogen. One embodiment provides the compound of Formula (I) or (II), or pharmaceutically acceptable salt, solvate, N-oxide, or deuteroisotope thereof, wherein R¹ is optionally substituted C1-C6 alkyl. One embodiment provides the compound of Formula (I) or (II), or pharmaceutically acceptable salt, solvate, N-oxide, or deuteroisotope thereof, wherein R¹ is CH₃ or CD₃.

One embodiment provides the compound of Formula (I) or (II), or pharmaceutically acceptable salt, solvate, N-oxide, or deuteroisotope thereof, wherein R² is optionally substituted C1-C3 alkyl. One embodiment provides the compound of Formula (I) or (II), or pharmaceutically acceptable salt, solvate, N-oxide, or deuteroisotope thereof, wherein the optionally substituted C1-C3 alkyl is substituted with a halogen. One embodiment provides the compound of Formula (I) or (II), or pharmaceutically acceptable salt, solvate, N-oxide, or deuteroisotope thereof, wherein the optionally substituted C1-C3 alkyl is —CHF₂.

One embodiment provides the compound of Formula (I) or (II), or pharmaceutically acceptable salt, solvate, N-oxide, or deuteroisotope thereof, wherein R⁴ is hydrogen. One embodiment provides the compound of Formula (I) or (II), or pharmaceutically acceptable salt, solvate, N-oxide, or deuteroisotope thereof, wherein R⁴ is halogen.

One embodiment provides the compound of Formula (I) or (II), or pharmaceutically acceptable salt, solvate, N-oxide, or deuteroisotope thereof, wherein R³ is hydrogen.

One embodiment provides the compound of Formula (I) or (II), or pharmaceutically acceptable salt, solvate, N-oxide, or deuteroisotope thereof, wherein V is N. One embodiment provides the compound of Formula (I) or (II), or pharmaceutically acceptable salt, solvate, N-oxide, or deuteroisotope thereof, wherein V is C—R¹¹. One embodiment provides the compound of Formula (I) or (II), or pharmaceutically acceptable salt, solvate, N-oxide, or deuteroisotope thereof, wherein V is C—R¹¹ and R¹¹ is hydrogen.

One embodiment provides the compound of Formula (I) or (II), or pharmaceutically acceptable salt, solvate, N-oxide, or deuteroisotope thereof, wherein W is C—H. One embodiment provides the compound of Formula (I) or (II), or pharmaceutically acceptable salt, solvate, N-oxide, or deuteroisotope thereof, wherein W is N.

One embodiment provides the compound of Formula (I) or (II), or pharmaceutically acceptable salt, solvate, N-oxide, or deuteroisotope thereof, wherein X is C—H. One embodiment provides the compound of Formula (I) or (II), or pharmaceutically acceptable salt, solvate, N-oxide, or deuteroisotope thereof, wherein X is N. One embodiment provides the compound of Formula (I) or (II), or pharmaceutically acceptable salt, solvate, N-oxide, or deuteroisotope thereof, wherein X is C—H and W is C—H.

One embodiment provides the compound of Formula (I) or (II), or pharmaceutically acceptable salt, solvate, N-oxide, or deuteroisotope thereof, wherein Group A is optionally substituted C1-C6 alkyl. One embodiment provides the compound of Formula (I) or (II), or pharmaceutically acceptable salt, solvate, N-oxide, or deuteroisotope thereof, wherein the optionally substituted C1-C6 alkyl is substituted with at least an amino group or a hydroxy group. One embodiment provides the compound of Formula (I) or (II), or pharmaceutically acceptable salt, solvate, N-oxide, or deuteroisotope thereof, wherein Group A is optionally substituted C1-C6 alkyl further substituted with —O-(optionally substituted heterocyclyl). One embodiment provides the compound of Formula (I) or (II), or pharmaceutically acceptable salt, solvate, N-oxide, or deuteroisotope thereof, wherein optionally substituted C1-C6 alkyl further substituted with —O-(optionally substituted heterocyclyl) is substituted with an —O—(N-methylpiperidine).

One embodiment provides the compound of Formula (I) or (II), or pharmaceutically acceptable salt, solvate, N-oxide, or deuteroisotope thereof, wherein Group A is optionally substituted C3-C6 carbocyclyl. One embodiment provides the compound of Formula (I) or (II), or pharmaceutically acceptable salt, solvate, N-oxide, or deuteroisotope thereof, wherein the optionally substituted C3-C6 carbocyclyl is substituted with at least an amino group or a hydroxy group.

One embodiment provides the compound of Formula (I) or (II), or pharmaceutically acceptable salt, solvate, N-oxide, or deuteroisotope thereof, wherein Group A is optionally substituted 5- or 6-membered heterocyclyl. One embodiment provides the compound of Formula (I) or (II), or pharmaceutically acceptable salt, solvate, N-oxide, or deuteroisotope thereof, wherein the optionally substituted 5- or 6-membered heterocyclyl is an optionally substituted tetrahydrofuran, or optionally substituted tetrahydropyran. One embodiment provides the compound of Formula (I) or (II), or pharmaceutically acceptable salt, solvate, N-oxide, or deuteroisotope thereof, wherein the optionally substituted 5- or 6-membered heterocyclyl is an optionally substituted piperidine, optionally substituted morpholine, optionally substituted piperazine, or optionally substituted pyrrolidine.

One embodiment provides the compound of Formula (I) or (II), or pharmaceutically acceptable salt, solvate, N-oxide, or deuteroisotope thereof, wherein Group A is optionally substituted 5-membered heteroaryl. One embodiment provides the compound of Formula (I) or (II), or pharmaceutically acceptable salt, solvate, N-oxide, or deuteroisotope thereof, wherein the optionally substituted 5-membered heteroaryl is selected from optionally substituted pyrazole, optionally substituted thiazole, optionally substituted oxazole, optionally substituted imidazole, or optionally substituted triazole.

One embodiment provides the compound of Formula (I) or (II), or pharmaceutically acceptable salt, solvate, N-oxide, or deuteroisotope thereof, wherein Group A is optionally substituted 6-membered heteroaryl. One embodiment provides the compound of Formula (I) or (II), or pharmaceutically acceptable salt, solvate, N-oxide, or deuteroisotope thereof, wherein the optionally substituted 6-membered heteroaryl is selected from optionally substituted pyridine or optionally substituted pyrimidine.

One embodiment provides the compound of Formula (I) or (II), or pharmaceutically acceptable salt, solvate, N-oxide, or deuteroisotope thereof, wherein Group A is optionally substituted aryl. One embodiment provides the compound of Formula (I) or (II), or pharmaceutically acceptable salt, solvate, N-oxide, or deuteroisotope thereof, wherein the optionally substituted aryl is optionally substituted phenyl.

One embodiment provides the compound of Formula (I) or (II), or pharmaceutically acceptable salt, solvate, N-oxide, or deuteroisotope thereof, wherein Group A is optionally substituted C4-C7 carbocyclylalkyl. One embodiment provides the compound of Formula (I) or (II), or pharmaceutically acceptable salt, solvate, N-oxide, or deuteroisotope thereof, wherein the optionally substituted C4-C7 carbocyclylalkyl is —CH₂-(optionally substituted carbocyclyl).

One embodiment provides the compound of Formula (I) or (II), or pharmaceutically acceptable salt, solvate, N-oxide, or deuteroisotope thereof, wherein Group A is optionally substituted heterocyclyl further substituted with an optionally substituted heterocyclyl. One embodiment provides the compound of Formula (I) or (II), or pharmaceutically acceptable salt, solvate, N-oxide, or deuteroisotope thereof, wherein the optionally substituted heterocyclyl further substituted with an optionally substituted carbocyclyl or heterocyclyl is selected from:

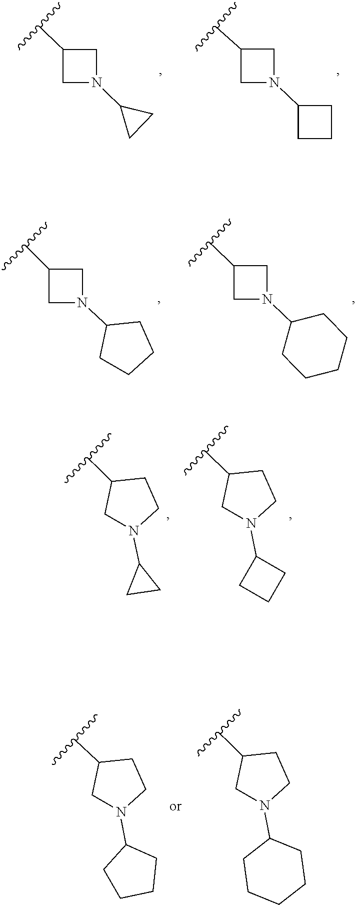

One embodiment provides the compound of Formula (I) or (II), or pharmaceutically acceptable salt, solvate, N-oxide, or deuteroisotope thereof, wherein the optionally substituted heterocyclyl further substituted with an optionally substituted carbocyclyl or heterocyclyl is selected from:

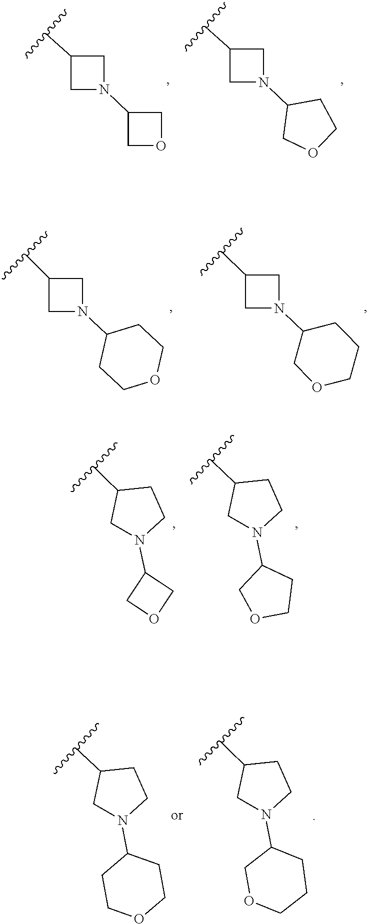

One embodiment provides a TNF-α inhibitory compound, or a pharmaceutically acceptable salt, solvate, or N-oxide thereof, having a structure presented in Table 1.

TABLE 1

| Synthetic | | |
|---|---|---|
| Chemistry | | |
| Example | Compound Structure | Compound Name |
| 1 | 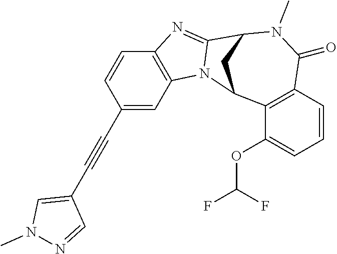 [SMILES: CN1C(=O)c2cccc(OC(F)F)c2[C@H]2C[C@@H]1c1nc3ccc(C#Cc4cnn(C)c4)cc3n12] | (1R,11R)-18-(difluoromethoxy)-12- methyl-5-[2-(1-methylpyrazol-4- yl)ethynyl]-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 2 | 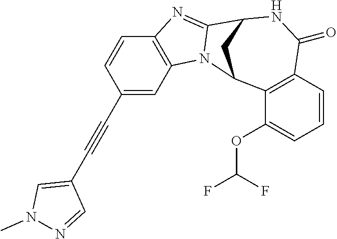 [SMILES: Cn1cc(C#Cc2ccc3nc4n(c3c2)[C@@H]2C[C@H]4NC(=O)c3cccc(OC(F)F)c32)cn1] | (1R,11R)-18-(difluoromethoxy)-5-[2-(1- methylpyrazol-4-yl)ethynyl]-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 3 | 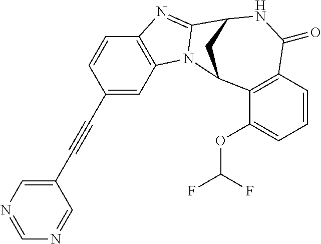 [SMILES: O=C1N[C@@H]2C[C@H](c3c(OC(F)F)cccc31)n1c2nc2ccc(C#Cc3cncnc3)cc21] | (1R,11R)-18-(difluoromethoxy)-5-[2- (pyrimidin-5-yl)ethynyl]-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 4 | 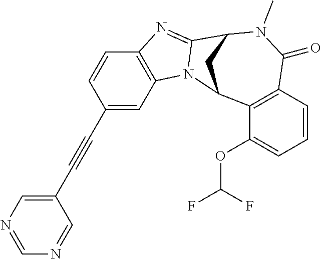 [SMILES: CN1C(=O)c2cccc(OC(F)F)c2[C@H]2C[C@@H]1c1nc3ccc(C#Cc4cncnc4)cc3n12] | (1R,11R)-18-(difluoromethoxy)-12- methyl-5-[2-(pyrimidin-5-yl)ethynyl]- 2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 5 | 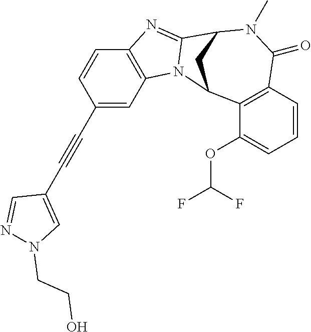 [SMILES: CN1C(=O)c2cccc(OC(F)F)c2[C@H]2C[C@@H]1c1nc3ccc(C#Cc4cnn(CCO)c4)cc3n12] | (1R,11R)-18-(difluoromethoxy)-5-{2-[1- (2-hydroxyethyl)pyrazol-4-yl]ethynyl}- 12-methyl-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 6 | 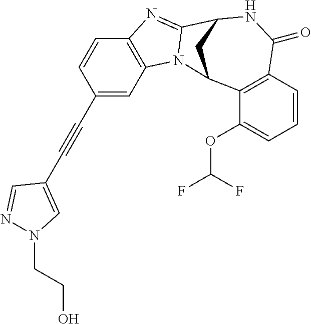 [SMILES: O=C1N[C@@H]2C[C@H](c3c(OC(F)F)cccc31)n1c2nc2ccc(C#Cc3cnn(CCO)c3)cc21] | (1R,11R)-18-(difluoromethoxy)-5-{2-[1- (2-hydroxyethyl)pyrazol-4-yl]ethynyl}- 2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 7 | 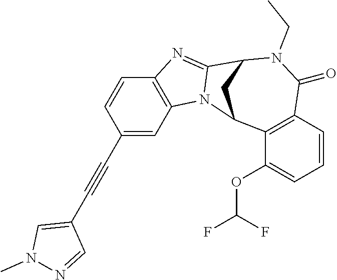 [SMILES: CCN1C(=O)c2cccc(OC(F)F)c2[C@H]2C[C@@H]1c1nc3ccc(C#Cc4cnn(C)c4)cc3n12] | (1R,11R)-18-(difluoromethoxy)-12- ethyl-5-[2-(1-methylpyrazol-4- yl)ethynyl]-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 8 | 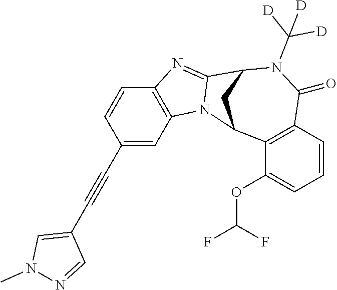 [SMILES: [2H]C([2H])([2H])N1C(=O)c2cccc(OC(F)F)c2[C@H]2C[C@@H]1c1nc3ccc(C#Cc4cnn(C)c4)cc3n12] | (7R,14R)-1-(difluoromethoxy)-6- (methyl-d3)-11-((1-methyl-1H-pyrazol- 4-yl)ethynyl)-6,7-dihydro-7,14- methanobenzo[f]benzo[4,5]imidazo[1,2- a][1,4]diazocin-5(14H)-one |
| 9 | 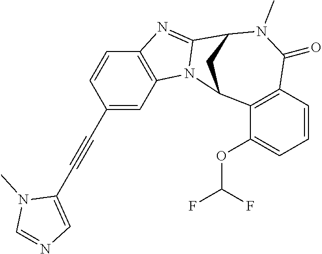 [SMILES: CN1C(=O)c2cccc(OC(F)F)c2[C@H]2C[C@@H]1c1nc3ccc(C#Cc4cncn4C)cc3n12] | (1R,11R)-18-(difluoromethoxy)-12- methyl-5-[2-(3-methylimidazol-4- yl)ethynyl]-2,9,12- triazapentacyclo[9.8.1.0^{∧}{2,10}.0^{∧}{3,8}. 0^{∧}{14,19}]icosa-3(8),4,6,9,14(19),15,17- heptaen-13-one |
| 10 | 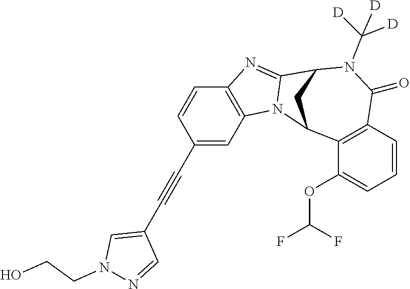 [SMILES: [2H]C([2H])([2H])N1C(=O)c2cccc(OC(F)F)c2[C@H]2C[C@@H]1c1nc3ccc(C#Cc4cnn(CCO)c4)cc3n12] | (7R,14R)-1-(difluoromethoxy)-11-((1-(2- hydroxyethyl)-1H-pyrazol-4-yl)ethynyl)- 6-(methyl-d3)-6,7-dihydro-7,14- methanobenzo[f]benzo[4,5]imidazo[1,2- a][1,4]diazocin-5(14H)-one |
| 11 | 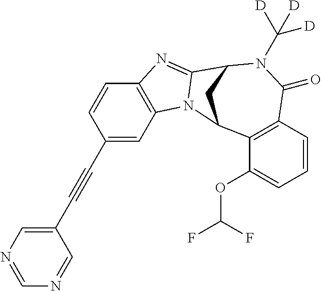 [SMILES: [2H]C([2H])([2H])N1C(=O)c2cccc(OC(F)F)c2[C@H]2C[C@@H]1c1nc3ccc(C#Cc4cncnc4)cc3n12] | (7R,14R)-1-(difluoromethoxy)-6- (methyl-d3)-11-(pyrimidin-5-ylethynyl)- 6,7-dihydro-7,14- methanobenzo[f]benzo[4,5]imidazo[1,2- a][1,4]diazocin-5(14H)-one |
| 12 | 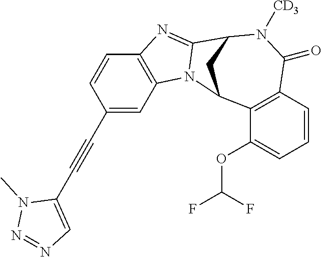 [SMILES: [2H]C([2H])([2H])N1C(=O)c2cccc(OC(F)F)c2[C@H]2C[C@@H]1c1nc3ccc(C#Cc4cnnn4C)cc3n12] | (7R,14R)-1-(difluoromethoxy)-6- (methyl-d3)-11-((1-methyl-1H-1,2,3- triazol-5-yl)ethynyl)-6,7-dihydro-7,14- methanobenzo[f]benzo[4,5]imidazo[1,2- a][1,4]diazocin-5(14H)-one |
| 13 | 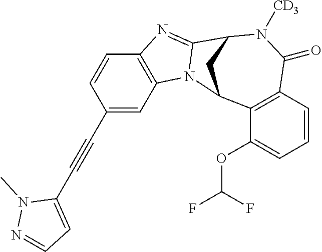 [SMILES: [2H]C([2H])([2H])N1C(=O)c2cccc(OC(F)F)c2[C@H]2C[C@@H]1c1nc3ccc(C#Cc4ccnn4C)cc3n12] | (7R,14R)-1-(difluoromethoxy)-6- (methyl-d3)-11-((1-methyl-1H-pyrazol- 5-yl)ethynyl)-6,7-dihydro-7,14- methanobenzo[f]benzo[4,5]imidazo[1,2- a][1,4]diazocin-5(14H)-one |
| 14 | 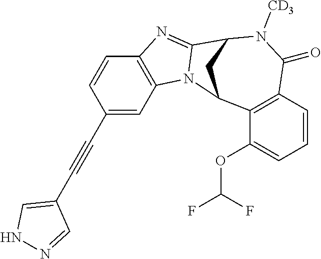 [SMILES: [2H]C([2H])([2H])N1C(=O)c2cccc(OC(F)F)c2[C@H]2C[C@@H]1c1nc3ccc(C#Cc4cn[nH]c4)cc3n12] | (7R,14R)-11-((1H-pyrazol-4-yl)ethynyl)- 1-(difluoromethoxy)-6-(methyl-d3)-6,7- dihydro-7,14- methanobenzo[f]benzo[4,5]imidazo[1,2- a][1,4]diazocin-5(14H)-one |
| 15 | 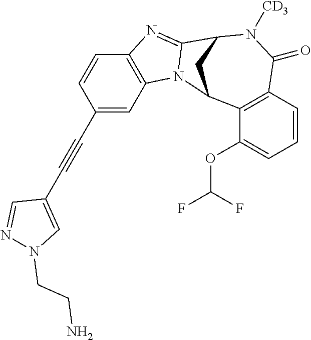 [SMILES: [2H]C([2H])([2H])N1C(=O)c2cccc(OC(F)F)c2[C@H]2C[C@@H]1c1nc3ccc(C#Cc4cnn(CCN)c4)cc3n12] | (7R,14R)-11-((1-(2-aminoethyl)-1H- pyrazol-4-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 16 | 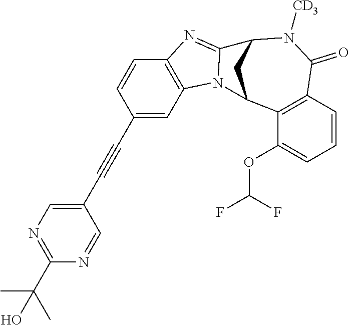 [SMILES: [2H]C([2H])([2H])N1C(=O)c2cccc(OC(F)F)c2[C@H]2C[C@@H]1c1nc3ccc(C#Cc4cnc(C(C)(C)O)nc4)cc3n12] | (7R,14R)-1-(difluoromethoxy)-11-((2-(2- hydroxypropan-2-yl)pyrimidin-5- yl)ethynyl)-6-(methyl-d3)-6,7-dihydro- 7,14- methanobenzo[f]benzo[4,5]imidazo[1,2- a][1,4]diazocin-5(14H)-one |
| 17 | 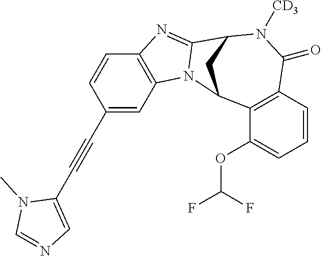 [SMILES: [2H]C([2H])([2H])N1C(=O)c2cccc(OC(F)F)c2[C@H]2C[C@@H]1c1nc3ccc(C#Cc4cncn4C)cc3n12] | (7R,14R)-1-(difluoromethoxy)-6- (methyl-d3)-11-((1-methyl-1H-imidazol- 5-yl)ethynyl)-6,7-dihydro-7,14- methanobenzo[f]benzo[4,5]imidazo[1,2- a][1,4]diazocin-5(14H)-one |
| 18 | 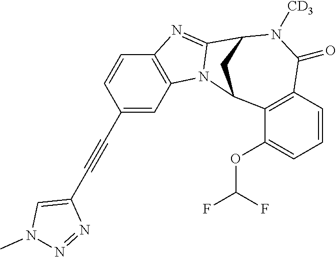 [SMILES: [2H]C([2H])([2H])N1C(=O)c2cccc(OC(F)F)c2[C@H]2C[C@@H]1c1nc3ccc(C#Cc4cn(C)nn4)cc3n12] | (7R,14R)-1-(difluoromethoxy)-6- (methyl-d3)-11-((1-methyl-1H-1,2,3- triazol-4-yl)ethynyl)-6,7-dihydro-7,14- methanobenzo[f]benzo[4,5]imidazo[1,2- a][1,4]diazocin-5(14H)-one |
| 19 | 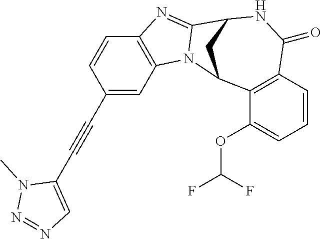 [SMILES: Cn1nncc1C#Cc1ccc2nc3n(c2c1)[C@@H]1C[C@H]3NC(=O)c2cccc(OC(F)F)c21] | (7R,14R)-1-(difluoromethoxy)-11-((1- methyl-1H-1,2,3-triazol-5-yl)ethynyl)- 6,7-dihydro-7,14- methanobenzo[f]benzo[4,5]imidazo[1,2- a][1,4]diazocin-5(14H)-one |
| 20 | 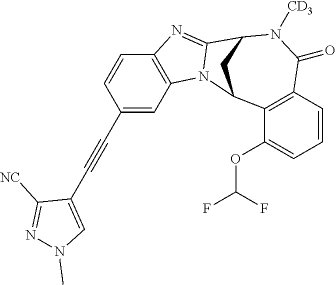 [SMILES: [2H]C([2H])([2H])N1C(=O)c2cccc(OC(F)F)c2[C@H]2C[C@@H]1c1nc3ccc(C#Cc4cn(C)nc4C#N)cc3n12] | 4-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)-1-methyl- 1H-pyrazole-3-carbonitrile |
| 21 | 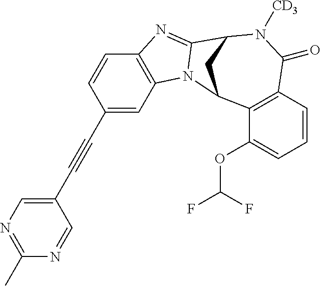 [SMILES: [2H]C([2H])([2H])N1C(=O)c2cccc(OC(F)F)c2[C@H]2C[C@@H]1c1nc3ccc(C#Cc4cnc(C)nc4)cc3n12] | (7R,14R)-1-(difluoromethoxy)-6- (methyl-d3)-11-((2-methylpyrimidin-5- yl)ethynyl)-6,7-dihydro-7,14- methanobenzo[f]benzo[4,5]imidazo[1,2- a][1,4]diazocin-5(14H)-one |
| 22 | 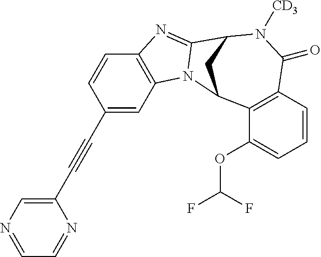 [SMILES: [2H]C([2H])([2H])N1C(=O)c2cccc(OC(F)F)c2[C@H]2C[C@@H]1c1nc3ccc(C#Cc4cnccn4)cc3n12] | (7R,14R)-1-(difluoromethoxy)-6- (methyl-d3)-11-(pyrazin-2-ylethynyl)- 6,7-dihydro-7,14- methanobenzo[f]benzo[4,5]imidazo[1,2- a][1,4]diazocin-5(14H)-one |
| 23 | 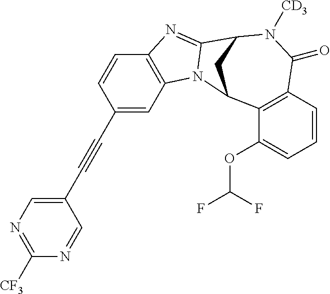 [SMILES: [2H]C([2H])([2H])N1C(=O)c2cccc(OC(F)F)c2[C@H]2C[C@@H]1c1nc3ccc(C#Cc4cnc(C(F)(F)F)nc4)cc3n12] | (7R,14R)-1-(difluoromethoxy)-6- (methyl-d3)-11-((2- (trifluoromethyl)pyrimidin-5-yl)ethynyl)- 6,7-dihydro-7,14- methanobenzo[f]benzo[4,5]imidazo[1,2- a][1,4]diazocin-5(14H)-one |
| 24 | 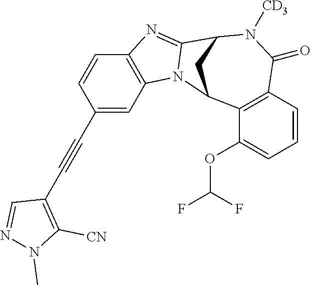 [SMILES: [2H]C([2H])([2H])N1C(=O)c2cccc(OC(F)F)c2[C@H]2C[C@@H]1c1nc3ccc(C#Cc4cnn(C)c4C#N)cc3n12] | 4-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)-1-methyl- 1H-pyrazole-5-carbonitrile |
| 25 | 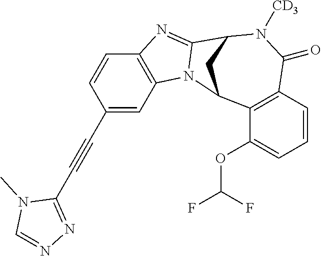 [SMILES: [2H]C([2H])([2H])N1C(=O)c2cccc(OC(F)F)c2[C@H]2C[C@@H]1c1nc3ccc(C#Cc4nncn4C)cc3n12] | (7R,14R)-1-(difluoromethoxy)-6- (methyl-d3)-11-((4-methyl-4H-1,2,4- triazol-3-yl)ethynyl)-6,7-dihydro-7,14- methanobenzo[f]benzo[4,5]imidazo[1,2- a][1,4]diazocin-5(14H)-one |
| 26 | 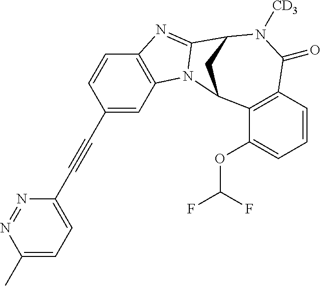 [SMILES: [2H]C([2H])([2H])N1C(=O)c2cccc(OC(F)F)c2[C@H]2C[C@@H]1c1nc3ccc(C#Cc4ccc(C)nn4)cc3n12] | (7R,14R)-1-(difluoromethoxy)-6- (methyl-d3)-11-((6-methylpyridazin-3- yl)ethynyl)-6,7-dihydro-7,14- methanobenzo[f]benzo[4,5]imidazo[1,2- a][1,4]diazocin-5(14H)-one |
| 27 | 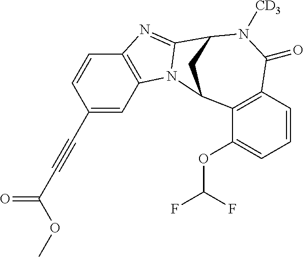 [SMILES: [2H]C([2H])([2H])N1C(=O)c2cccc(OC(F)F)c2[C@H]2C[C@@H]1c1nc3ccc(C#CC(=O)OC)cc3n12] | methyl 3-((7R,14R)-1- (difluoromethoxy)-6-(methyl-d3)-5-oxo- 5,6,7,14-tetrahydro-7,14- methanobenzo[f]benzo[4,5]imidazo[1,2- a][1,4]diazocin-11-yl)propiolate |
| 28 | 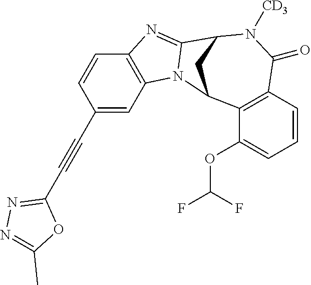 [SMILES: [2H]C([2H])([2H])N1C(=O)c2cccc(OC(F)F)c2[C@H]2C[C@@H]1c1nc3ccc(C#Cc4nnc(C)o4)cc3n12] | (7R,14R)-1-(difluoromethoxy)-6- (methyl-d3)-11-((5-methyl-1,3,4- oxadiazol-2-yl)ethynyl)-6,7-dihydro- 7,14- methanobenzo[f]benzo[4,5]imidazo[1,2- a][1,4]diazocin-5(14H)-one |
| 29 | 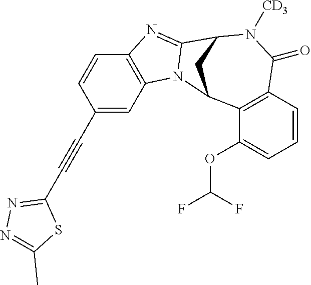 [SMILES: [2H]C([2H])([2H])N1C(=O)c2cccc(OC(F)F)c2[C@H]2C[C@@H]1c1nc3ccc(C#Cc4nnc(C)s4)cc3n12] | (7R,14R)-1-(difluoromethoxy)-6- (methyl-d3)-11-((5-methyl-1,3,4- thiadiazol-2-yl)ethynyl)-6,7-dihydro- 7,14- methanobenzo[f]benzo[4,5]imidazo[1,2- a][1,4]diazocin-5(14H)-one |
| 30 | 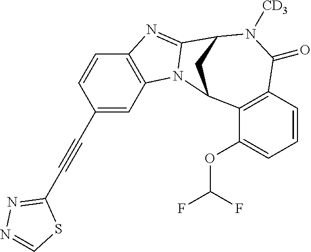 [SMILES: [2H]C([2H])([2H])N1C(=O)c2cccc(OC(F)F)c2[C@H]2C[C@@H]1c1nc3ccc(C#Cc4nncs4)cc3n12] | (7R,14R)-11-((1,3,4-thiadiazol-2- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 31 | 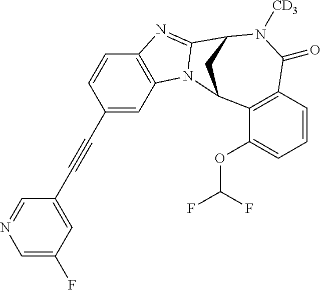 [SMILES: [2H]C([2H])([2H])N1C(=O)c2cccc(OC(F)F)c2[C@H]2C[C@@H]1c1nc3ccc(C#Cc4cncc(F)c4)cc3n12] | (7R,14R)-1-(difluoromethoxy)-11-((5- fluoropyridin-3-yl)ethynyl)-6-(methyl- d3)-6,7-dihydro-7,14- methanobenzo[f]benzo[4,5]imidazo[1,2- a][1,4]diazocin-5(14H)-one |
| 32 | 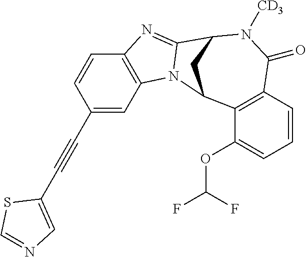 [SMILES: [2H]C([2H])([2H])N1C(=O)c2cccc(OC(F)F)c2[C@H]2C[C@@H]1c1nc3ccc(C#Cc4cncs4)cc3n12] | (7R,14R)-1-(difluoromethoxy)-6- (methyl-d3)-11-(thiazol-5-ylethynyl)-6,7- dihydro-7,14- methanobenzo[f]benzo[4,5]imidazo[1,2- a][1,4]diazocin-5(14H)-one |
| 33 | 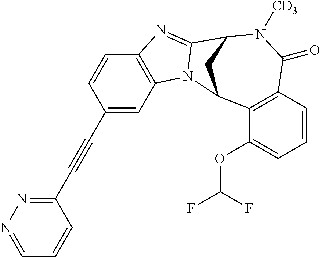 [SMILES: [2H]C([2H])([2H])N1C(=O)c2cccc(OC(F)F)c2[C@H]2C[C@@H]1c1nc3ccc(C#Cc4cccnn4)cc3n12] | (7R,14R)-1-(difluoromethoxy)-6- (methyl-d3)-11-(pyridazin-3-ylethynyl)- 6,7-dihydro-7,14- methanobenzo[f]benzo[4,5]imidazo[1,2- a][1,4]diazocin-5(14H)-one |
| 34 | 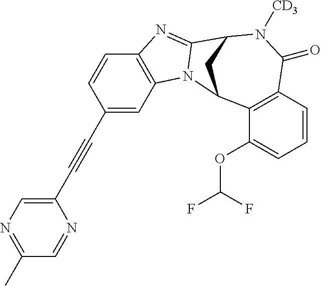 [SMILES: [2H]C([2H])([2H])N1C(=O)c2cccc(OC(F)F)c2[C@H]2C[C@@H]1c1nc3ccc(C#Cc4cnc(C)cn4)cc3n12] | (7R,14R)-1-(difluoromethoxy)-6- (methyl-d3)-11-((5-methylpyrazin-2- yl)ethynyl)-6,7-dihydro-7,14- methanobenzo[f]benzo[4,5]imidazo[1,2- a][1,4]diazocin-5(14H)-one |
| 35 | 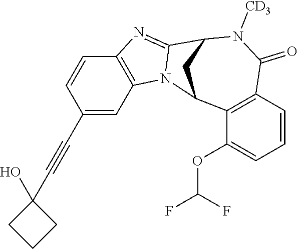 [SMILES: [2H]C([2H])([2H])N1C(=O)c2cccc(OC(F)F)c2[C@H]2C[C@@H]1c1nc3ccc(C#CC4(O)CCC4)cc3n12] | (7R,14R)-1-(difluoromethoxy)-11-((1- hydroxycyclobutyl)ethynyl)-6-(methyl- d3)-6,7-dihydro-7,14- methanobenzo[f]benzo[4,5]imidazo[1,2- a][1,4]diazocin-5(14H)-one |
| 36 | 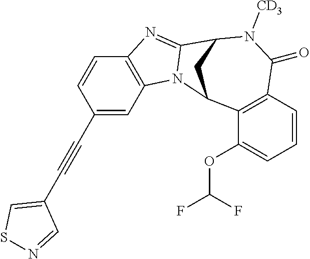 [SMILES: [2H]C([2H])([2H])N1C(=O)c2cccc(OC(F)F)c2[C@H]2C[C@@H]1c1nc3ccc(C#Cc4cnsc4)cc3n12] | (7R,14R)-1-(difluoromethoxy)-11- (isothiazol-4-ylethynyl)-6-(methyl-d3)- 6,7-dihydro-7,14- methanobenzo[f]benzo[4,5]imidazo[1,2- a][1,4]diazocin-5(14H)-one |
| 37 | 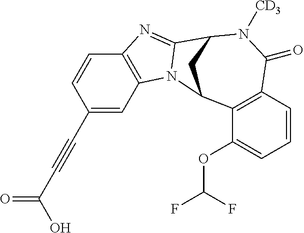 [SMILES: [2H]C([2H])([2H])N1C(=O)c2cccc(OC(F)F)c2[C@H]2C[C@@H]1c1nc3ccc(C#CC(=O)O)cc3n12] | 3-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)propiolic acid |
| 38 | 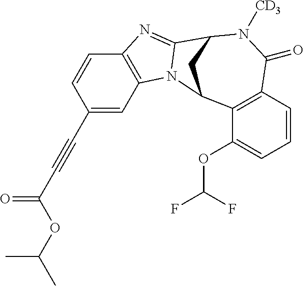 [SMILES: [2H]C([2H])([2H])N1C(=O)c2cccc(OC(F)F)c2[C@H]2C[C@@H]1c1nc3ccc(C#CC(=O)OC(C)C)cc3n12] | isopropyl 3-((7R,14R)-1- (difluoromethoxy)-6-(methyl-d3)-5-oxo- 5,6,7,14-tetrahydro-7,14- methanobenzo[f]benzo[4,5]imidazo[1,2- a][1,4]diazocin-11-yl)propiolate |
| 39 | 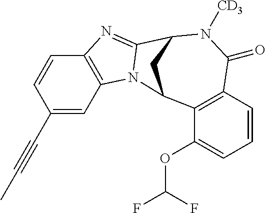 [SMILES: [2H]C([2H])([2H])N1C(=O)c2cccc(OC(F)F)c2[C@H]2C[C@@H]1c1nc3ccc(C#CC)cc3n12] | (7R,14R)-1-(difluoromethoxy)-6- (methyl-d3)-11-(prop-1-yn-1-yl)-6,7- dihydro-7,14- methanobenzo[f]benzo[4,5]imidazo[1,2- a][1,4]diazocin-5(14H)-one |
| 40 | 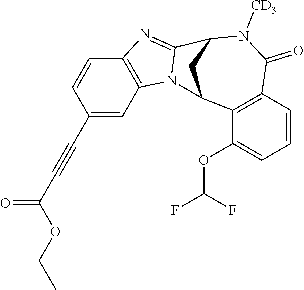 [SMILES: [2H]C([2H])([2H])N1C(=O)c2cccc(OC(F)F)c2[C@H]2C[C@@H]1c1nc3ccc(C#CC(=O)OCC)cc3n12] | ethyl 3-((7R,14R)-1-(difluoromethoxy)- 6-(methyl-d3)-5-oxo-5,6,7,14-tetrahydro- 7,14- methanobenzo[f]benzo[4,5]imidazo[1,2- a][1,4]diazocin-11-yl)propiolate |
| 41 | 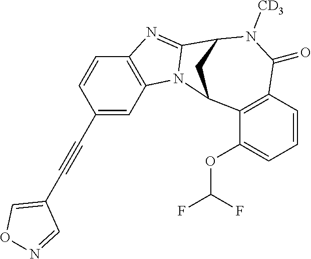 [SMILES: [2H]C([2H])([2H])N1C(=O)c2cccc(OC(F)F)c2[C@H]2C[C@@H]1c1nc3ccc(C#Cc4cnoc4)cc3n12] | (7R,14R)-1-(difluoromethoxy)-11- (isoxazol-4-ylethynyl)-6-(methyl-d3)- 6,7-dihydro-7,14- methanobenzo[f]benzo[4,5]imidazo[1,2- a][1,4]diazocin-5(14H)-one |
| 42 | 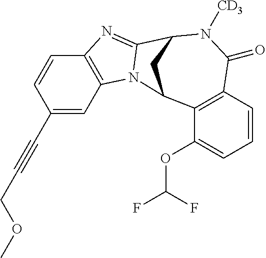 [SMILES: [2H]C([2H])([2H])N1C(=O)c2cccc(OC(F)F)c2[C@H]2C[C@@H]1c1nc3ccc(C#CCOC)cc3n12] | (7R,14R)-1-(difluoromethoxy)-11-(3- methoxyprop-1-yn-1-yl)-6-(methyl-d3)- 6,7-dihydro-7,14- methanobenzo[f]benzo[4,5]imidazo[1,2- a][1,4]diazocin-5(14H)-one |
| 43 | 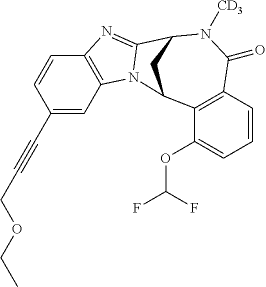 [SMILES: [2H]C([2H])([2H])N1C(=O)c2cccc(OC(F)F)c2[C@H]2C[C@@H]1c1nc3ccc(C#CCOCC)cc3n12] | (7R,14R)-1-(difluoromethoxy)-11-(3- ethoxyprop-1-yn-1-yl)-6-(methyl-d3)- 6,7-dihydro-7,14- methanobenzo[f]benzo[4,5]imidazo[1,2- a][1,4]diazocin-5(14H)-one |
| 44 | 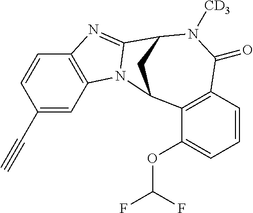 [SMILES: [2H]C([2H])([2H])N1C(=O)c2cccc(OC(F)F)c2[C@H]2C[C@@H]1c1nc3ccc(C#C)cc3n12] | (7R,14R)-1-(difluoromethoxy)-11- ethynyl-6-(methyl-d3)-6,7-dihydro-7,14- methanobenzo[f]benzo[4,5]imidazo[1,2- a][1,4]diazocin-5(14H)-one |
| 45 | 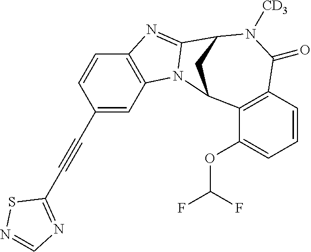 [SMILES: [2H]C([2H])([2H])N1C(=O)c2cccc(OC(F)F)c2[C@H]2C[C@@H]1c1nc3ccc(C#Cc4ncns4)cc3n12] | (7R,14R)-11-((1,2,4-thiadiazol-5- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 46 | 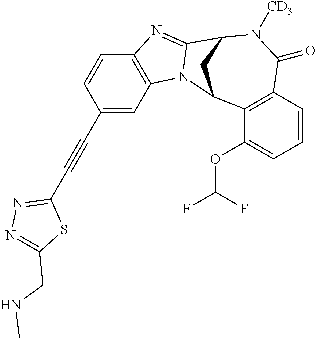 [SMILES: [2H]C([2H])([2H])N1C(=O)c2cccc(OC(F)F)c2[C@H]2C[C@@H]1c1nc3ccc(C#Cc4nnc(CNC)s4)cc3n12] | (7R,14R)-1-(difluoromethoxy)-6- (methyl-d3)-11-((5- ((methylamino)methyl)-1,3,4-thiadiazol- 2-yl)ethynyl)-6,7-dihydro-7,14- methanobenzo[f]benzo[4,5]imidazo[1,2- a][1,4]diazocin-5(14H)-one |
| 47 | 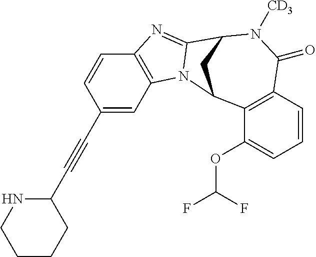 [SMILES: [2H]C([2H])([2H])N1C(=O)c2cccc(OC(F)F)c2[C@H]2C[C@@H]1c1nc3ccc(C#CC4CCCCN4)cc3n12] | (7R,14R)-1-(difluoromethoxy)-6- (methyl-d3)-11-(piperidin-2-ylethynyl)- 6,7-dihydro-7,14- methanobenzo[f]benzo[4,5]imidazo[1,2- a][1,4]diazocin-5(14H)-one |
| 48 | 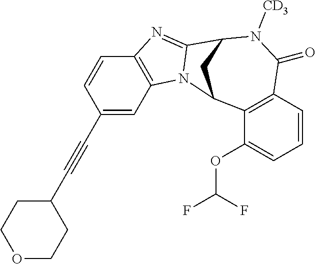 [SMILES: [2H]C([2H])([2H])N1C(=O)c2cccc(OC(F)F)c2[C@H]2C[C@@H]1c1nc3ccc(C#CC4CCOCC4)cc3n12] | (7R,14R)-1-(difluoromethoxy)-6- (methyl-d3)-11-((tetrahydro-2H-pyran-4- yl)ethynyl)-6,7-dihydro-7,14- methanobenzo[f]benzo[4,5]imidazo[1,2- a][1,4]diazocin-5(14H)-one |
| 49 | 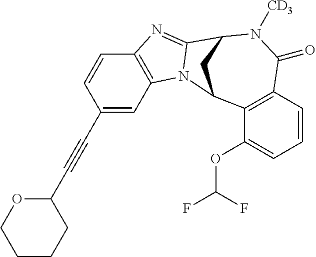 [SMILES: [2H]C([2H])([2H])N1C(=O)c2cccc(OC(F)F)c2[C@H]2C[C@@H]1c1nc3ccc(C#CC4CCCCO4)cc3n12] | (7R,14R)-1-(difluoromethoxy)-6- (methyl-d3)-11-((tetrahydro-2H-pyran-2- yl)ethynyl)-6,7-dihydro-7,14- methanobenzo[f]benzo[4,5]imidazo[1,2- a][1,4]diazocin-5(14H)-one |
| 50 | 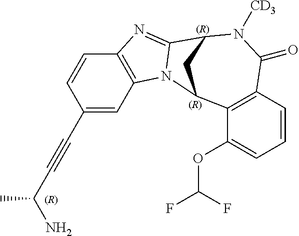 | (7R,14R)-11-((R)-3-aminobut-1-yn-1- yl)-1-(difluoromethoxy)-6-(methyl-d3)- 6,7-dihydro-7,14- methanobenzo[f]benzo[4,5]imidazo[1,2- a][1,4]diazocin-5(14H)-one |
| 51 | 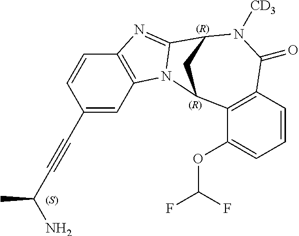 | (7R,14R)-11-((S)-3-aminobut-1-yn-1-yl)- 1-(difluoromethoxy)-6-(methyl-d3)-6,7- dihydro-7,14- methanobenzo[f]benzo[4,5]imidazo[1,2- a][1,4]diazocin-5(14H)-one |
| 52 | 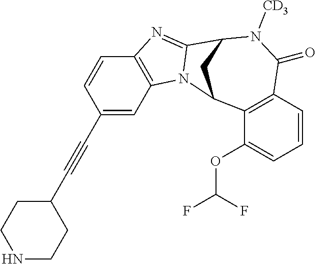 [SMILES: [2H]C([2H])([2H])N1C(=O)c2cccc(OC(F)F)c2[C@H]2C[C@@H]1c1nc3ccc(C#CC4CCNCC4)cc3n12] | (7R,14R)-1-(difluoromethoxy)-6- (methyl-d3)-11-(piperidin-4-ylethynyl)- 6,7-dihydro-7,14- methanobenzo[f]benzo[4,5]imidazo[1,2- a][1,4]diazocin-5(14H)-one |
| 53 | 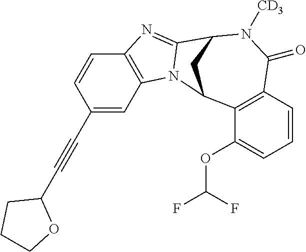 [SMILES: [2H]C([2H])([2H])N1C(=O)c2cccc(OC(F)F)c2[C@H]2C[C@@H]1c1nc3ccc(C#CC4CCCO4)cc3n12] | (7R,14R)-1-(difluoromethoxy)-6- (methyl-d3)-11-((tetrahydrofuran-2- yl)ethynyl)-6,7-dihydro-7,14- methanobenzo[f]benzo[4,5]imidazo[1,2- a][1,4]diazocin-5(14H)-one |
| 54 | 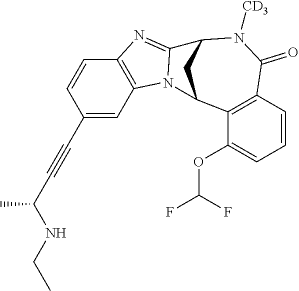 [SMILES: [2H]C([2H])([2H])N1C(=O)c2cccc(OC(F)F)c2[C@H]2C[C@@H]1c1nc3ccc(C#C[C@@H](C)NCC)cc3n12] | (7R,14R)-1-(difluoromethoxy)-11-((R)- 3-(ethylamino)but-1-yn-1-yl)-6-(methyl- d3)-6,7-dihydro-7,14- methanobenzo[f]benzo[4,5]imidazo[1,2- a][1,4]diazocin-5(14H)-one |
| 55 | 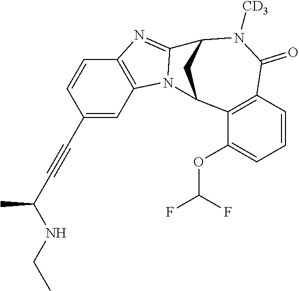 [SMILES: [2H]C([2H])([2H])N1C(=O)c2cccc(OC(F)F)c2[C@H]2C[C@@H]1c1nc3ccc(C#C[C@H](C)NCC)cc3n12] | (7R,14R)-1-(difluoromethoxy)-11-((S)-3- (ethylamino)but-1-yn-1-yl)-6-(methyl- d3)-6,7-dihydro-7,14- methanobenzo[f]benzo[4,5]imidazo[1,2- a][1,4]diazocin-5(14H)-one |
| 56 | 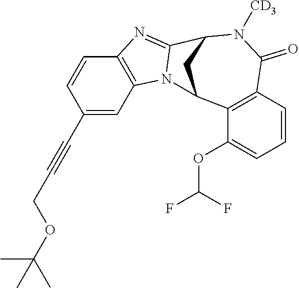 [SMILES: [2H]C([2H])([2H])N1C(=O)c2cccc(OC(F)F)c2[C@H]2C[C@@H]1c1nc3ccc(C#CCOC(C)(C)C)cc3n12] | (7R,14R)-11-(3-(tert-butoxy)prop-1-yn- 1-yl)-1-(difluoromethoxy)-6-(methyl- d3)-6,7-dihydro-7,14- methanobenzo[f]benzo[4,5]imidazo[1,2- a][1,4]diazocin-5(14H)-one |
| 57 | 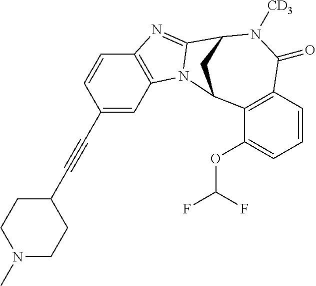 [SMILES: [2H]C([2H])([2H])N1C(=O)c2cccc(OC(F)F)c2[C@H]2C[C@@H]1c1nc3ccc(C#CC4CCN(C)CC4)cc3n12] | (7R,14R)-1-(difluoromethoxy)-6- (methyl-d3)-11-((1-methylpiperidin-4- yl)ethynyl)-6,7-dihydro-7,14- methanobenzo[f]benzo[4,5]imidazo[1,2- a][1,4]diazocin-5(14H)-one |
| 58 | 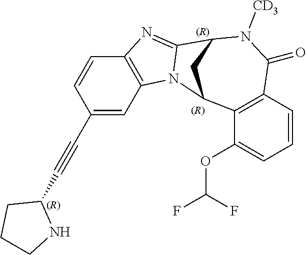 | (7R,14R)-1-(difluoromethoxy)-6- (methyl-d3)-11-(((R)-pyrrolidin-2- yl)ethynyl)-6,7-dihydro-7,14- methanobenzo[f]benzo[4,5]imidazo[1,2- a][1,4]diazocin-5(14H)-one |
| 59 | 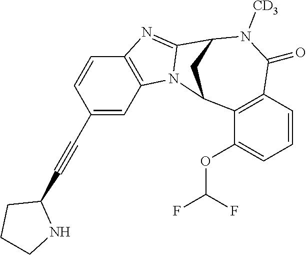 [SMILES: [2H]C([2H])([2H])N1C(=O)c2cccc(OC(F)F)c2[C@H]2C[C@@H]1c1nc3ccc(C#C[C@@H]4CCCN4)cc3n12] | (7R,14R)-1-(difluoromethoxy)-6- (methyl-d3)-11-(((S)-pyrrolidin-2- yl)ethynyl)-6,7-dihydro-7,14- methanobenzo[f]benzo[4,5]imidazo[1,2- a][1,4]diazocin-5(14H)-one |
| 60 | 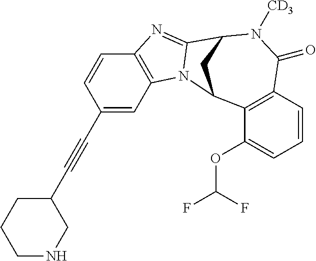 [SMILES: [2H]C([2H])([2H])N1C(=O)c2cccc(OC(F)F)c2[C@H]2C[C@@H]1c1nc3ccc(C#CC4CCCNC4)cc3n12] | (7R,14R)-1-(difluoromethoxy)-6- (methyl-d3)-11-(piperidin-3-ylethynyl)- 6,7-dihydro-7,14- methanobenzo[f]benzo[4,5]imidazo[1,2- a][1,4]diazocin-5(14H)-one |
| 61 | 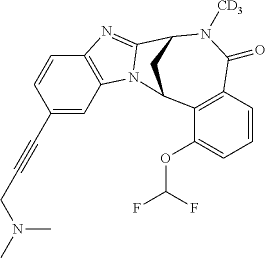 [SMILES: [2H]C([2H])([2H])N1C(=O)c2cccc(OC(F)F)c2[C@H]2C[C@@H]1c1nc3ccc(C#CCN(C)C)cc3n12] | (7R,14R)-1-(difluoromethoxy)-11-(3- (dimethylamino)prop-1-yn-1-yl)-6- (methyl-d3)-6,7-dihydro-7,14- methanobenzo[f]benzo[4,5]imidazo[1,2- a][1,4]diazocin-5(14H)-one |
| 62 | 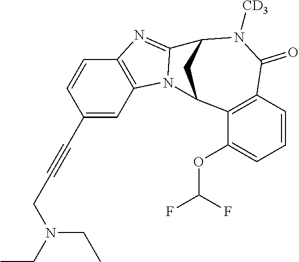 [SMILES: [2H]C([2H])([2H])N1C(=O)c2cccc(OC(F)F)c2[C@H]2C[C@@H]1c1nc3ccc(C#CCN(CC)CC)cc3n12] | (7R,14R)-11-(3-(diethylamino)prop-1- yn-1-yl)-1-(difluoromethoxy)-6-(methyl- d3)-6,7-dihydro-7,14- methanobenzo[f]benzo[4,5]imidazo[1,2- a][1,4]diazocin-5(14H)-one |
| 63 | 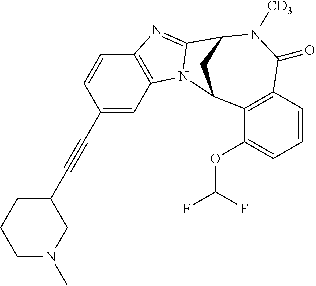 [SMILES: [2H]C([2H])([2H])N1C(=O)c2cccc(OC(F)F)c2[C@H]2C[C@@H]1c1nc3ccc(C#CC4CCCN(C)C4)cc3n12] | (7R,14R)-1-(difluoromethoxy)-6- (methyl-d3)-11-((1-methylpiperidin-3- yl)ethynyl)-6,7-dihydro-7,14- methanobenzo[f]benzo[4,5]imidazo[1,2- a][1,4]diazocin-5(14H)-one |
| 64 | 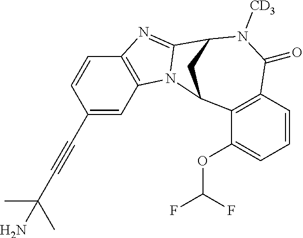 [SMILES: [2H]C([2H])([2H])N1C(=O)c2cccc(OC(F)F)c2[C@H]2C[C@@H]1c1nc3ccc(C#CC(C)(C)N)cc3n12] | (7R,14R)-11-(3-amino-3-methylbut-1- yn-1-yl)-1-(difluoromethoxy)-6-(methyl- d3)-6,7-dihydro-7,14- methanobenzo[f]benzo[4,5]imidazo[1,2- a][1,4]diazocin-5(14H)-one |
| 65 | 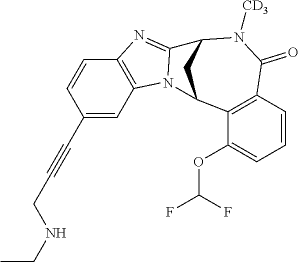 [SMILES: [2H]C([2H])([2H])N1C(=O)c2cccc(OC(F)F)c2[C@H]2C[C@@H]1c1nc3ccc(C#CCNCC)cc3n12] | (7R,14R)-1-(difluoromethoxy)-11-(3- (ethylamino)prop-1-yn-1-yl)-6-(methyl- d3)-6,7-dihydro-7,14- methanobenzo[f]benzo[4,5]imidazo[1,2- a][1,4]diazocin-5(14H)-one |
| 66 | 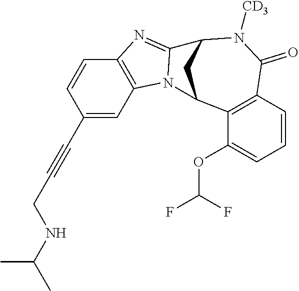 [SMILES: [2H]C([2H])([2H])N1C(=O)c2cccc(OC(F)F)c2[C@H]2C[C@@H]1c1nc3ccc(C#CCNC(C)C)cc3n12] | (7R,14R)-1-(difluoromethoxy)-11-(3- (isopropylamino)prop-1-yn-1-yl)-6- (methyl-d3)-6,7-dihydro-7,14- methanobenzo[f]benzo[4,5]imidazo[1,2- a][1,4]diazocin-5(14H)-one |
| 67 | 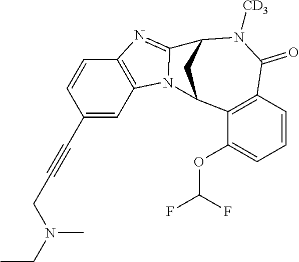 [SMILES: [2H]C([2H])([2H])N1C(=O)c2cccc(OC(F)F)c2[C@H]2C[C@@H]1c1nc3ccc(C#CCN(C)CC)cc3n12] | (7R,14R)-1-(difluoromethoxy)-11-(3- (ethyl(methyl)amino)prop-1-yn-1-yl)-6- (methyl-d3)-6,7-dihydro-7,14- methanobenzo[f]benzo[4,5]imidazo[1,2- a][1,4]diazocin-5(14H)-one |
| 68 | 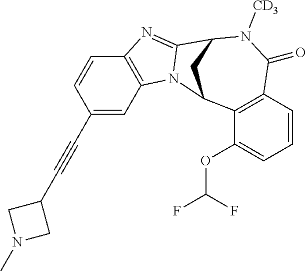 [SMILES: [2H]C([2H])([2H])N1C(=O)c2cccc(OC(F)F)c2[C@H]2C[C@@H]1c1nc3ccc(C#CC4CN(C)C4)cc3n12] | (7R,14R)-1-(difluoromethoxy)-6- (methyl-d3)-11-((1-methylazetidin-3- yl)ethynyl)-6,7-dihydro-7,14- methanobenzo[f]benzo[4,5]imidazo[1,2- a][1,4]diazocin-5(14H)-one |
| 69 | 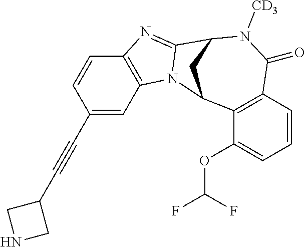 [SMILES: [2H]C([2H])([2H])N1C(=O)c2cccc(OC(F)F)c2[C@H]2C[C@@H]1c1nc3ccc(C#CC4CNC4)cc3n12] | (7R,14R)-11-(azetidin-3-ylethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 70 | 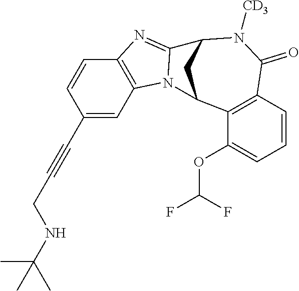 [SMILES: [2H]C([2H])([2H])N1C(=O)c2cccc(OC(F)F)c2[C@H]2C[C@@H]1c1nc3ccc(C#CCNC(C)(C)C)cc3n12] | (7R,14R)-11-(3-(tert-butylamino)prop-1- yn-1-yl)-1-(difluoromethoxy)-6-(methyl- d3)-6,7-dihydro-7,14- methanobenzo[f]benzo[4,5]imidazo[1,2- a][1,4]diazocin-5(14H)-one |
| 71 | 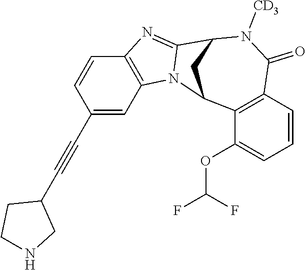 [SMILES: [2H]C([2H])([2H])N1C(=O)c2cccc(OC(F)F)c2[C@H]2C[C@@H]1c1nc3ccc(C#CC4CCNC4)cc3n12] | (7R,14R)-1-(difluoromethoxy)-6- (methyl-d3)-11-(pyrrolidin-3-ylethynyl)- 6,7-dihydro-7,14- methanobenzo[f]benzo[4,5]imidazo[1,2- a][1,4]diazocin-5(14H)-one |
| 72 | 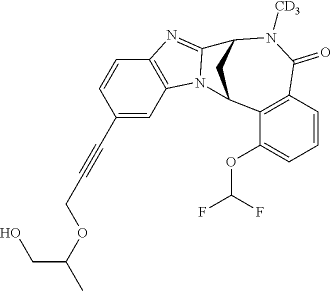 [SMILES: [2H]C([2H])([2H])N1C(=O)c2cccc(OC(F)F)c2[C@H]2C[C@@H]1c1nc3ccc(C#CCOC(C)CO)cc3n12] | (7R,14R)-1-(difluoromethoxy)-11-(3-((1- hydroxypropan-2-yl)oxy)prop-1-yn-1- yl)-6-(methyl-d3)-6,7-dihydro-7,14- methanobenzo[f]benzo[4,5]imidazo[1,2- a][1,4]diazocin-5(14H)-one |
| 73 | 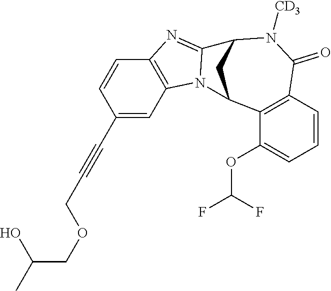 [SMILES: [2H]C([2H])([2H])N1C(=O)c2cccc(OC(F)F)c2[C@H]2C[C@@H]1c1nc3ccc(C#CCOCC(C)O)cc3n12] | (7R,14R)-1-(difluoromethoxy)-11-(3-(2- hydroxypropoxy)prop-1-yn-1-yl)-6- (methyl-d3)-6,7-dihydro-7,14- methanobenzo[f]benzo[4,5]imidazo[1,2- a][1,4]diazocin-5(14H)-one |
| 74 | 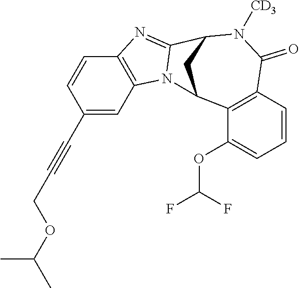 [SMILES: [2H]C([2H])([2H])N1C(=O)c2cccc(OC(F)F)c2[C@H]2C[C@@H]1c1nc3ccc(C#CCOC(C)C)cc3n12] | (7R,14R)-1-(difluoromethoxy)-11-(3- isopropoxyprop-1-yn-1-yl)-6-(methyl- d3)-6,7-dihydro-7,14- methanobenzo[f]benzo[4,5]imidazo[1,2- a][1,4]diazocin-5(14H)-one |
| 75 | 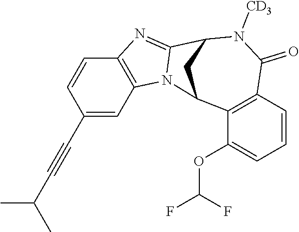 [SMILES: [2H]C([2H])([2H])N1C(=O)c2cccc(OC(F)F)c2[C@H]2C[C@@H]1c1nc3ccc(C#CC(C)C)cc3n12] | (7R,14R)-1-(difluoromethoxy)-6- (methyl-d3)-11-(3-methylbut-1-yn-1-yl)- 6,7-dihydro-7,14- methanobenzo[f]benzo[4,5]imidazo[1,2- a][1,4]diazocin-5(14H)-one |
| 76 | 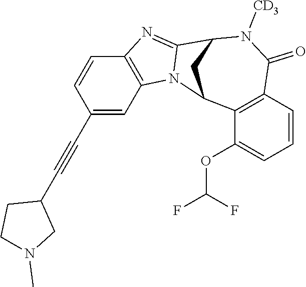 [SMILES: [2H]C([2H])([2H])N1C(=O)c2cccc(OC(F)F)c2[C@H]2C[C@@H]1c1nc3ccc(C#CC4CCN(C)C4)cc3n12] | (7R,14R)-1-(difluoromethoxy)-6- (methyl-d3)-11-((1-methylpyrrolidin-3- yl)ethynyl)-6,7-dihydro-7,14- methanobenzo[f]benzo[4,5]imidazo[1,2- a][1,4]diazocin-5(14H)-one |
| 77 | 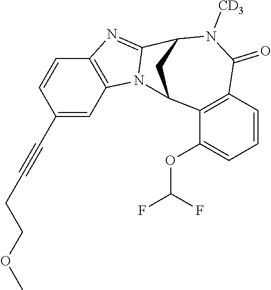 [SMILES: [2H]C([2H])([2H])N1C(=O)c2cccc(OC(F)F)c2[C@H]2C[C@@H]1c1nc3ccc(C#CCCOC)cc3n12] | (7R,14R)-1-(difluoromethoxy)-11-(4- methoxybut-1-yn-1-yl)-6-(methyl-d3)- 6,7-dihydro-7,14- methanobenzo[f]benzo[4,5]imidazo[1,2- a][1,4]diazocin-5(14H)-one |
| 78 | 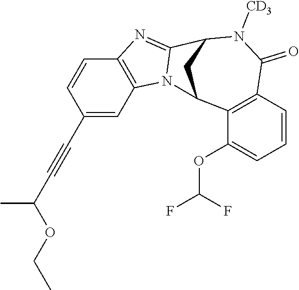 [SMILES: [2H]C([2H])([2H])N1C(=O)c2cccc(OC(F)F)c2[C@H]2C[C@@H]1c1nc3ccc(C#CC(C)OCC)cc3n12] | (7R,14R)-1-(difluoromethoxy)-11-(3- ethoxybut-1-yn-1-yl)-6-(methyl-d3)-6,7- dihydro-7,14- methanobenzo[f]benzo[4,5]imidazo[1,2- a][1,4]diazocin-5(14H)-one |
| 79 | 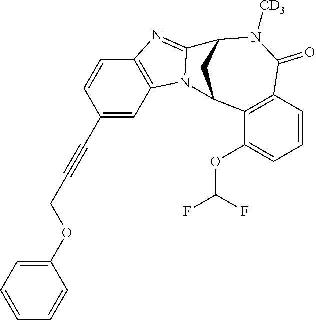 [SMILES: [2H]C([2H])([2H])N1C(=O)c2cccc(OC(F)F)c2[C@H]2C[C@@H]1c1nc3ccc(C#CCOc4ccccc4)cc3n12] | (7R,14R)-1-(difluoromethoxy)-6- (methyl-d3)-11-(3-phenoxyprop-1-yn-1- yl)-6,7-dihydro-7,14- methanobenzo[f]benzo[4,5]imidazo[1,2- a][1,4]diazocin-5(14H)-one |
| 80 | 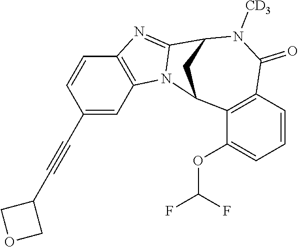 [SMILES: [2H]C([2H])([2H])N1C(=O)c2cccc(OC(F)F)c2[C@H]2C[C@@H]1c1nc3ccc(C#CC4COC4)cc3n12] | (7R,14R)-1-(difluoromethoxy)-6- (methyl-d3)-11-(oxetan-3-ylethynyl)-6,7- dihydro-7,14- methanobenzo[f]benzo[4,5]imidazo[1,2- a][1,4]diazocin-5(14H)-one |
| 81 | 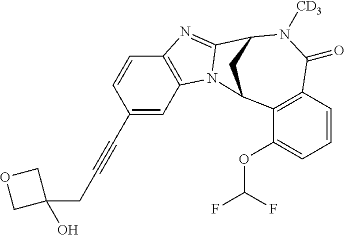 [SMILES: [2H]C([2H])([2H])N1C(=O)c2cccc(OC(F)F)c2[C@H]2C[C@@H]1c1nc3ccc(C#CCC4(O)COC4)cc3n12] | (7R,14R)-1-(difluoromethoxy)-11-(3-(3- hydroxyoxetan-3-yl)prop-1-yn-1-yl)-6- (methyl-d3)-6,7-dihydro-7,14- methanobenzo[f]benzo[4,5]imidazo[1,2- a][1,4]diazocin-5(14H)-one |
| 82 | 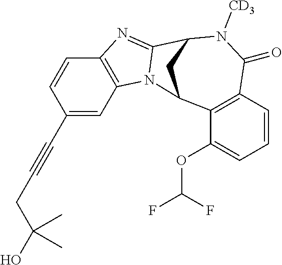 [SMILES: [2H]C([2H])([2H])N1C(=O)c2cccc(OC(F)F)c2[C@H]2C[C@@H]1c1nc3ccc(C#CCC(C)(C)O)cc3n12] | (7R,14R)-1-(difluoromethoxy)-11-(4- hydroxy-4-methylpent-1-yn-1-yl)-6- (methyl-d3)-6,7-dihydro-7,14- methanobenzo[f]benzo[4,5]imidazo[1,2- a][1,4]diazocin-5(14H)-one |
| 83 | 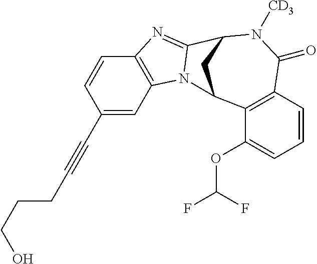 | (7R,14R)-1-(difluoromethoxy)-11-(5- hydroxypent-1-yn-1-yl)-6-(methyl-d3)- 6,7-dihydro-7,14- methanobenzo[f]benzo[4,5]imidazo[1,2- a][1,4]diazocin-5(14H)-one |
| 84 | 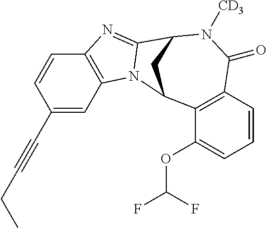 [SMILES: [2H]C([2H])([2H])N1C(=O)c2cccc(OC(F)F)c2[C@H]2C[C@@H]1c1nc3ccc(C#CCC)cc3n12] | (7R,14R)-11-(but-1-yn-1-yl)-1- (difluoromethoxy)-6-(methyl-d3)-6,7- dihydro-7,14- methanobenzo[f]benzo[4,5]imidazo[1,2- a][1,4]diazocin-5(14H)-one |
| 85 | 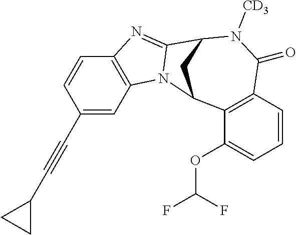 [SMILES: [2H]C([2H])([2H])N1C(=O)c2cccc(OC(F)F)c2[C@H]2C[C@@H]1c1nc3ccc(C#CC4CC4)cc3n12] | (7R,14R)-11-(cyclopropylethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 86 | 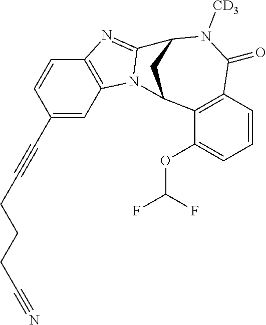 [SMILES: [2H]C([2H])([2H])N1C(=O)c2cccc(OC(F)F)c2[C@H]2C[C@@H]1c1nc3ccc(C#CCCCC#N)cc3n12] | 6-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)hex-5-ynenitrile |
| 87 | 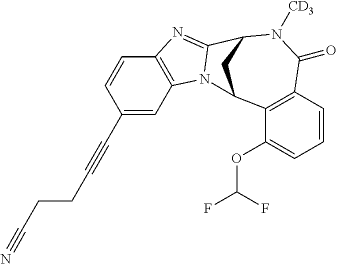 [SMILES: [2H]C([2H])([2H])N1C(=O)c2cccc(OC(F)F)c2[C@H]2C[C@@H]1c1nc3ccc(C#CCCC#N)cc3n12] | 5-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)pent-4-ynenitrile |
| 88 | 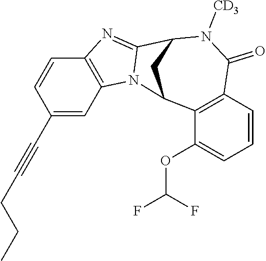 [SMILES: [2H]C([2H])([2H])N1C(=O)c2cccc(OC(F)F)c2[C@H]2C[C@@H]1c1nc3ccc(C#CCCC)cc3n12] | (7R,14R)-1-(difluoromethoxy)-6- (methyl-d3)-11-(pent-1-yn-1-yl)-6,7- dihydro-7,14- methanobenzo[f]benzo[4,5]imidazo[1,2- a][1,4]diazocin-5(14H)-one |
| 89 | 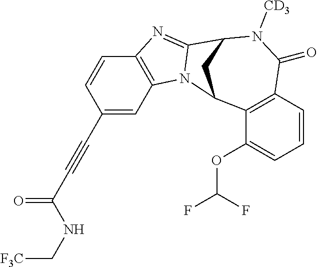 [SMILES: [2H]C([2H])([2H])N1C(=O)c2cccc(OC(F)F)c2[C@H]2C[C@@H]1c1nc3ccc(C#CC(=O)NCC(F)(F)F)cc3n12] | 3-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)-N-(2,2,2- trifluoroethyl)propiolamide |
| 90 | 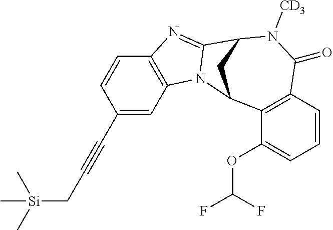 [SMILES: [2H]C([2H])([2H])N1C(=O)c2cccc(OC(F)F)c2[C@H]2C[C@@H]1c1nc3ccc(C#CC[Si](C)(C)C)cc3n12] | (7R,14R)-1-(difluoromethoxy)-6- (methyl-d3)-11-(3-(trimethylsilyl)prop-1- yn-1-yl)-6,7-dihydro-7,14- methanobenzo[f]benzo[4,5]imidazo[1,2- a][1,4]diazocin-5(14H)-one |
| 91 | 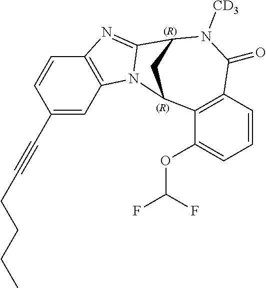 | (7R,14R)-1-(difluoromethoxy)-11-(hex- 1-yn-1-yl)-6-(methyl-d3)-6,7-dihydro- 7,14- methanobenzo[f]benzo[4,5]imidazo[1,2- a][1,4]diazocin-5(14H)-one |
| 92 | 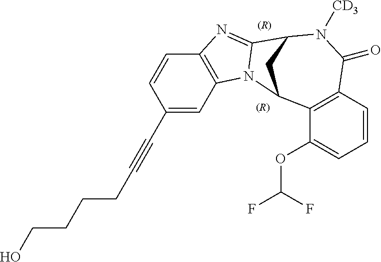 | (7R,14R)-1-(difluoromethoxy)-11-(6- hydroxyhex-1-yn-1-yl)-6-(methyl-d3)- 6,7-dihydro-7,14- methanobenzo[f]benzo[4,5]imidazo[1,2- a][1,4]diazocin-5(14H)-one |
| 93 | 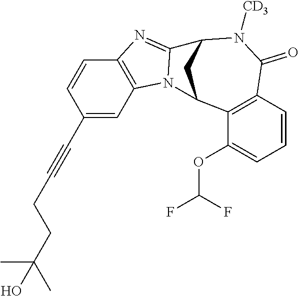 [SMILES: [2H]C([2H])([2H])N1C(=O)c2cccc(OC(F)F)c2[C@H]2C[C@@H]1c1nc3ccc(C#CCCC(C)(C)O)cc3n12] | (7R,14R)-1-(difluoromethoxy)-11-(5- hydroxy-5-methylhex-1-yn-1-yl)-6- (methyl-d3)-6,7-dihydro-7,14- methanobenzo[f]benzo[4,5]imidazo[1,2- a][1,4]diazocin-5(14H)-one |
| 94 | 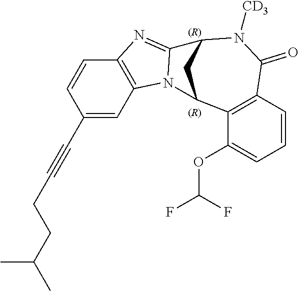 | (7R,14R)-1-(difluoromethoxy)-6- (methyl-d3)-11-(5-methylhex-1-yn-1-yl)- 6,7-dihydro-7,14- methanobenzo[f]benzo[4,5]imidazo[1,2- a][1,4]diazocin-5(14H)-one |
| 95 | 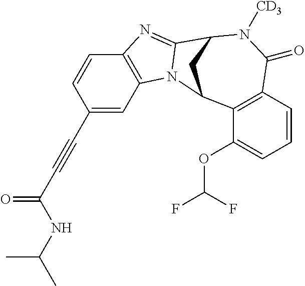 [SMILES: [2H]C([2H])([2H])N1C(=O)c2cccc(OC(F)F)c2[C@H]2C[C@@H]1c1nc3ccc(C#CC(=O)NC(C)C)cc3n12] | 3-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)-N- isopropylpropiolamide |
| 96 | 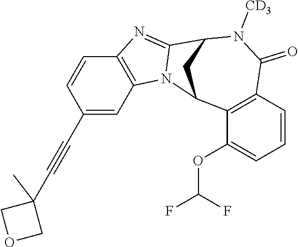 [SMILES: [2H]C([2H])([2H])N1C(=O)c2cccc(OC(F)F)c2[C@H]2C[C@@H]1c1nc3ccc(C#CC4(C)COC4)cc3n12] | (7R,14R)-1-(difluoromethoxy)-6- (methyl-d3)-11-((3-methyloxetan-3- yl)ethynyl)-6,7-dihydro-7,14- methanobenzo[f]benzo[4,5]imidazo[1,2- a][1,4]diazocin-5(14H)-one |
| 97 | 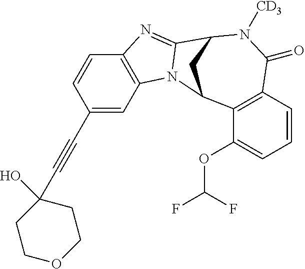 [SMILES: [2H]C([2H])([2H])N1C(=O)c2cccc(OC(F)F)c2[C@H]2C[C@@H]1c1nc3ccc(C#CC4(O)CCOCC4)cc3n12] | (7R,14R)-1-(difluoromethoxy)-11-((4- hydroxytetrahydro-2H-pyran-4- yl)ethynyl)-6-(methyl-d3)-6,7-dihydro- 7,14- methanobenzo[f]benzo[4,5]imidazo[1,2- a][1,4]diazocin-5(14H)-one |
| 98 | 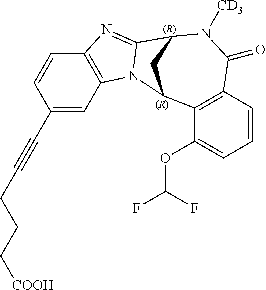 | 6-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)hex-5-ynoic acid |
| 99 | 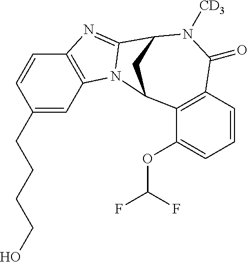 [SMILES: [2H]C([2H])([2H])N1C(=O)c2cccc(OC(F)F)c2[C@H]2C[C@@H]1c1nc3ccc(CCCCO)cc3n12] | (7R,14R)-1-(difluoromethoxy)-11-(4- hydroxybutyl)-6-(methyl-d3)-6,7- dihydro-7,14- methanobenzo[f]benzo[4,5]imidazo[1,2- a][1,4]diazocin-5(14H)-one |
| 100 | 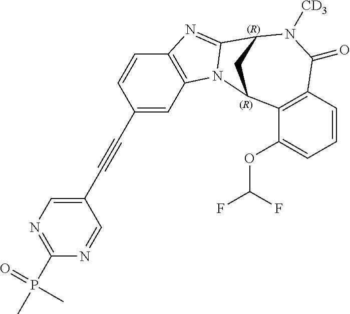 | (7R,14R)-1-(difluoromethoxy)-11-((2- (dimethylphosphoryl)pyrimidin-5- yl)ethynyl)-6-(methyl-d3)-6,7-dihydro- 7,14- methanobenzo[f]benzo[4,5]imidazo[1,2- a][1,4]diazocin-5(14H)-one |
| 101 | 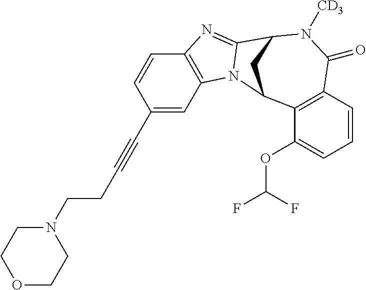 [SMILES: [2H]C([2H])([2H])N1C(=O)c2cccc(OC(F)F)c2[C@H]2C[C@@H]1c1nc3ccc(C#CCCN4CCOCC4)cc3n12] | (7R,14R)-1-(difluoromethoxy)-6- (methyl-d3)-11-(4-morpholinobut-1-yn- 1-yl)-6,7-dihydro-7,14- methanobenzo[f]benzo[4,5]imidazo[1,2- a][1,4]diazocin-5(14H)-one |
| 102 | 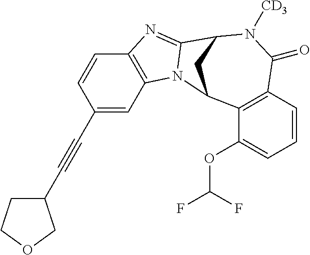 [SMILES: [2H]C([2H])([2H])N1C(=O)c2cccc(OC(F)F)c2[C@H]2C[C@@H]1c1nc3ccc(C#CC4CCOC4)cc3n12] | (7R,14R)-1-(difluoromethoxy)-6- (methyl-d3)-11-((tetrahydrofuran-3- yl)ethynyl)-6,7-dihydro-7,14- methanobenzo[f]benzo[4,5]imidazo[1,2- a][1,4]diazocin-5(14H)-one |
| 103 | 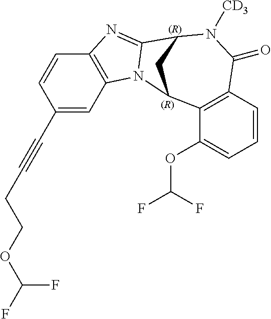 | (7R,14R)-1-(difluoromethoxy)-11-(4- (difluoromethoxy)but-1-yn-1-yl)-6- (methyl-d3)-6,7-dihydro-7,14- methanobenzo[f]benzo[4,5]imidazo[1,2- a][1,4]diazocin-5(14H)-one |
| 104 | 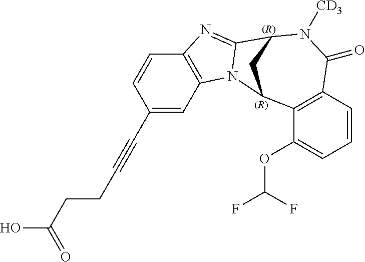 | 5-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)pent-4-ynoic acid |
| 105 | 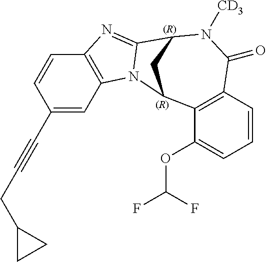 | (7R,14R)-11-(3-cyclopropylprop-1-yn-1- yl)-1-(difluoromethoxy)-6-(methyl-d3)- 6,7-dihydro-7,14- methanobenzo[f]benzo[4,5]imidazo[1,2- a][1,4]diazocin-5(14H)-one |
| 106 | 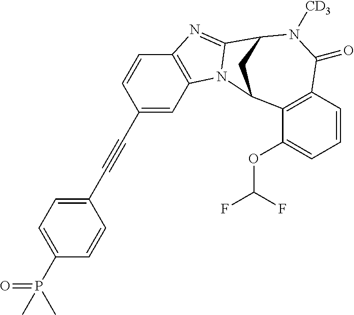 [SMILES: [2H]C([2H])([2H])N1C(=O)c2cccc(OC(F)F)c2[C@H]2C[C@@H]1c1nc3ccc(C#Cc4ccc(P(C)(C)=O)cc4)cc3n12] | (7R,14R)-1-(difluoromethoxy)-11-((4- (dimethylphosphoryl)phenyl)ethynyl)-6- (methyl-d3)-6,7-dihydro-7,14- methanobenzo[f]benzo[4,5]imidazo[1,2- a][1,4]diazocin-5(14H)-one |
| 107 | 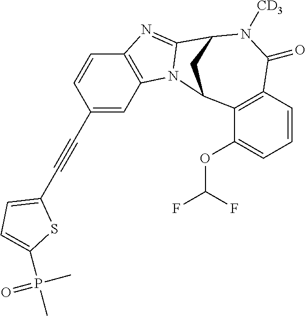 [SMILES: [2H]C([2H])([2H])N1C(=O)c2cccc(OC(F)F)c2[C@H]2C[C@@H]1c1nc3ccc(C#Cc4ccc(P(C)(C)=O)s4)cc3n12] | (7R,14R)-1-(difluoromethoxy)-11-((5- (dimethylphosphoryl)thiophen-2- yl)ethynyl)-6-(methyl-d3)-6,7-dihydro- 7,14- methanobenzo[f]benzo[4,5]imidazo[1,2- a][1,4]diazocin-5(14H)-one |
| 108 | 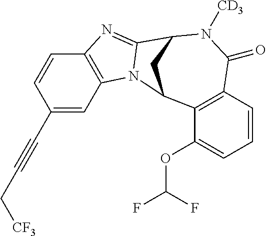 [SMILES: [2H]C([2H])([2H])N1C(=O)c2cccc(OC(F)F)c2[C@H]2C[C@@H]1c1nc3ccc(C#CCC(F)(F)F)cc3n12] | (7R,14R)-1-(difluoromethoxy)-6- (methyl-d3)-11-(4,4,4-trifluorobut-1-yn- 1-yl)-6,7-dihydro-7,14- methanobenzo[f]benzo[4,5]imidazo[1,2- a][1,4]diazocin-5(14H)-one |
| 109 | 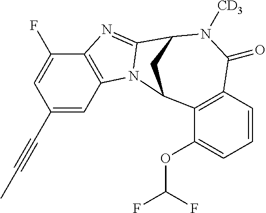 [SMILES: [2H]C([2H])([2H])N1C(=O)c2cccc(OC(F)F)c2[C@H]2C[C@@H]1c1nc3c(F)cc(C#CC)cc3n12] | (7R,14R)-1-(difluoromethoxy)-9-fluoro- 6-(methyl-d3)-11-(prop-1-yn-1-yl)-6,7- dihydro-7,14- methanobenzo[f]benzo[4,5]imidazo[1,2- a][1,4]diazocin-5(14H)-one |
| 110 | 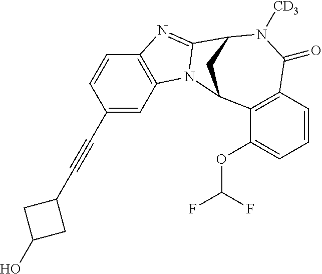 [SMILES: [2H]C([2H])([2H])N1C(=O)c2cccc(OC(F)F)c2[C@H]2C[C@@H]1c1nc3ccc(C#CC4CC(O)C4)cc3n12] | (7R,14R)-1-(difluoromethoxy)-11-((3- hydroxycyclobutyl)ethynyl)-6-(methyl- d3)-6,7-dihydro-7,14- methanobenzo[f]benzo[4,5]imidazo[1,2- a][1,4]diazocin-5(14H)-one |
| 111 | 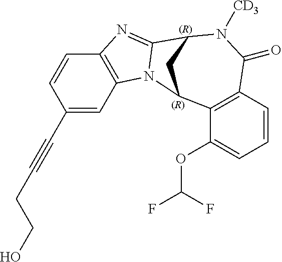 | (7R,14R)-1-(difluoromethoxy)-11-(4- hydroxybut-1-yn-1-yl)-6-(methyl-d3)- 6,7-dihydro-7,14- methanobenzo[f]benzo[4,5]imidazo[1,2- a][1,4]diazocin-5(14H)-one |
| 112 | 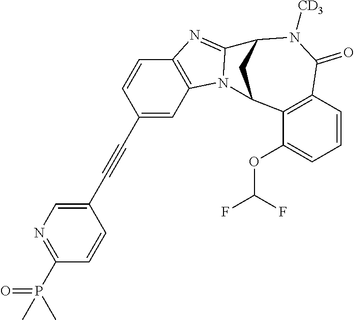 [SMILES: [2H]C([2H])([2H])N1C(=O)c2cccc(OC(F)F)c2[C@H]2C[C@@H]1c1nc3ccc(C#Cc4ccc(P(C)(C)=O)nc4)cc3n12] | (7R,14R)-1-(difluoromethoxy)-11-((6- (dimethylphosphoryl)pyridin-3- yl)ethynyl)-6-(methyl-d3)-6,7-dihydro- 7,14- methanobenzo[f]benzo[4,5]imidazo[1,2- a][1,4]diazocin-5(14H)-one |
| 113 | 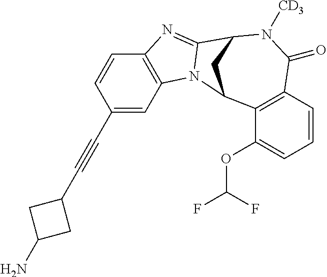 [SMILES: [2H]C([2H])([2H])N1C(=O)c2cccc(OC(F)F)c2[C@H]2C[C@@H]1c1nc3ccc(C#CC4CC(N)C4)cc3n12] | (7R,14R)-11-((3- aminocyclobutyl)ethynyl)-1- (difluoromethoxy)-6-(methyl-d₃)-6,7- dihydro-7,14- methanobenzo[f]benzo[4,5]imidazo[1,2- a][1,4]diazocin-5(14H)-one |
| 114 | 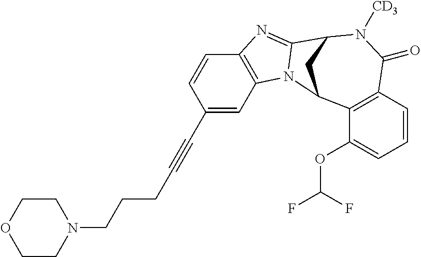 [SMILES: [2H]C([2H])([2H])N1C(=O)c2cccc(OC(F)F)c2[C@H]2C[C@@H]1c1nc3ccc(C#CCCCN4CCOCC4)cc3n12] | (7R,14R)-1-(difluoromethoxy)-6- (methyl-d₃)-11-(5-morpholinopent-1-yn- 1-yl)-6,7-dihydro-7,14- methanobenzo[f]benzo[4,5]imidazo[1,2- a][1,4]diazocin-5(14H)-one |
| 115 | 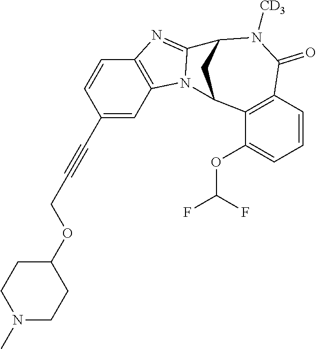 [SMILES: [2H]C([2H])([2H])N1C(=O)c2cccc(OC(F)F)c2[C@H]2C[C@@H]1c1nc3ccc(C#CCOC4CCN(C)CC4)cc3n12] | (7R,14R)-1-(difluoromethoxy)-6- (methyl-d₃)-11-(3-((1-methylpiperidin-4- yl)oxy)prop-1-yn-1-yl)-6,7-dihydro-7,14- methanobenzo[f]benzo[4,5]imidazo[1,2- a][1,4]diazocin-5(14H)-one |
| 116 | 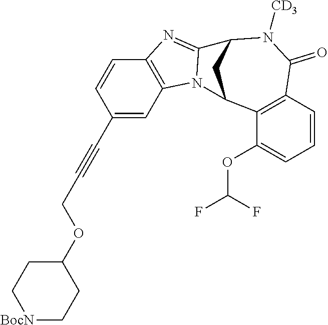 [SMILES: [2H]C([2H])([2H])N1C(=O)c2cccc(OC(F)F)c2[C@H]2C[C@@H]1c1nc3ccc(C#CCOC4CCN(C(=O)OC(C)(C)C)CC4)cc3n12] | tert-butyl 4-((3-((7R,14R)-1- (difluoromethoxy)-6-(methyl-d₃)-5-oxo- 5,6,7,14-tetrahydro-7,14- methanobenzo[f]benzo[4,5]imidazo[1,2- a][1,4]diazocin-11-yl)prop-2-yn-1- yl)oxy)piperidine-1-carboxylate |
| 117 | 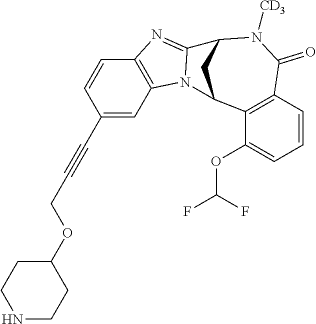 [SMILES: [2H]C([2H])([2H])N1C(=O)c2cccc(OC(F)F)c2[C@H]2C[C@@H]1c1nc3ccc(C#CCOC4CCNCC4)cc3n12] | (7R,14R)-1-(difluoromethoxy)-6- (methyl-d₃)-11-(3-(piperidin-4- yloxy)prop-1-yn-1-yl)-6,7-dihydro-7,14- methanobenzo[f]benzo[4,5]imidazo[1,2- a][1,4]diazocin-5(14H)-one |
| 118 | 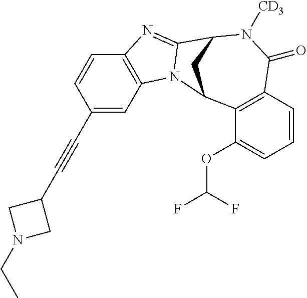 [SMILES: [2H]C([2H])([2H])N1C(=O)c2cccc(OC(F)F)c2[C@H]2C[C@@H]1c1nc3ccc(C#CC4CN(CC)C4)cc3n12] | (7R,14R)-1-(difluoromethoxy)-11-((1- ethylazetidin-3-yl)ethynyl)-6-(methyl- d₃)-6,7-dihydro-7,14- methanobenzo[f]benzo[4,5]imidazo[1,2- a][1,4]diazocin-5(14H)-one |
| 119 | 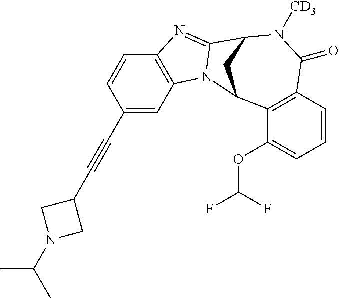 [SMILES: [2H]C([2H])([2H])N1C(=O)c2cccc(OC(F)F)c2[C@H]2C[C@@H]1c1nc3ccc(C#CC4CN(C(C)C)C4)cc3n12] | (7R,14R)-1-(difluoromethoxy)-11-((1- isopropylazetidin-3-yl)ethynyl)-6- (methyl-d3)-6,7-dihydro-7,14- methanobenzo[f]benzo[4,5]imidazo[1,2- a][1,4]diazocin-5(14H)-one |
| 120 | 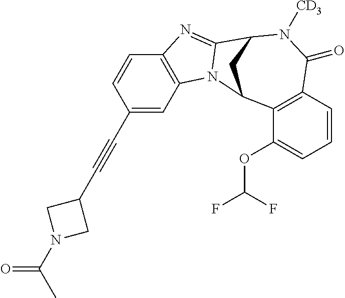 [SMILES: [2H]C([2H])([2H])N1C(=O)c2cccc(OC(F)F)c2[C@H]2C[C@@H]1c1nc3ccc(C#CC4CN(C(C)=O)C4)cc3n12] | (7R,14R)-11-((1-acetylazetidin-3- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 121 | 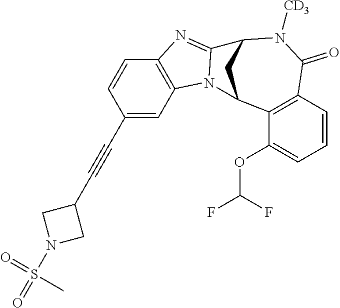 [SMILES: [2H]C([2H])([2H])N1C(=O)c2cccc(OC(F)F)c2[C@H]2C[C@@H]1c1nc3ccc(C#CC4CN(S(C)(=O)=O)C4)cc3n12] | (7R,14R)-1-(difluoromethoxy)-6- (methyl-d3)-11-((1- (methylsulfonyl)azetidin-3-yl)ethynyl)- 6,7-dihydro-7,14- methanobenzo[f]benzo[4,5]imidazo[1,2- a][1,4]diazocin-5(14H)-one |
| 122 | 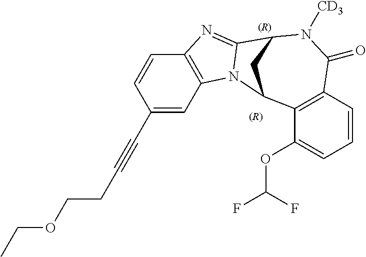 | (7R,14R)-1-(difluoromethoxy)-11-(4- ethoxybut-1-yn-1-yl)-6-(methyl-d₃)-6,7- dihydro-7,14- methanobenzo[f]benzo[4,5]imidazo[1,2- a][1,4]diazocin-5(14H)-one |
| 123 | 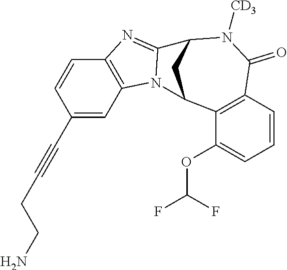 [SMILES: [2H]C([2H])([2H])N1C(=O)c2cccc(OC(F)F)c2[C@H]2C[C@@H]1c1nc3ccc(C#CCCN)cc3n12] | (7R,14R)-11-(4-aminobut-1-yn-1-yl)-1- (difluoromethoxy)-6-(methyl-d₃)-6,7- dihydro-7,14- methanobenzo[f]benzo[4,5]imidazo[1,2- a][1,4]diazocin-5(14H)-one |
| 124 | 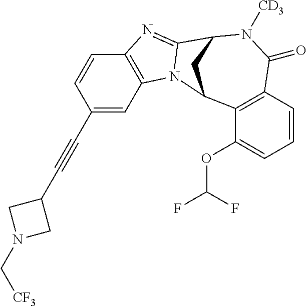 [SMILES: [2H]C([2H])([2H])N1C(=O)c2cccc(OC(F)F)c2[C@H]2C[C@@H]1c1nc3ccc(C#CC4CN(CC(F)(F)F)C4)cc3n12] | (7R,14R)-1-(difluoromethoxy)-6- (methyl-d₃)-11-((1-(2,2,2- trifluoroethyl)azetidin-3-yl)ethynyl)-6,7- dihydro-7,14- methanobenzo[f]benzo[4,5]imidazo[1,2- a][1,4]diazocin-5(14H)-one |
| 125 | 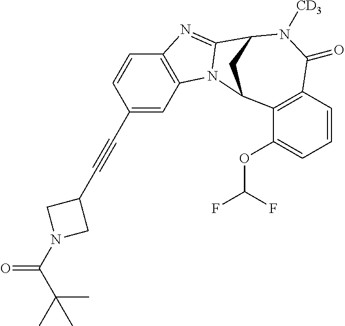 [SMILES: [2H]C([2H])([2H])N1C(=O)c2cccc(OC(F)F)c2[C@H]2C[C@@H]1c1nc3ccc(C#CC4CN(C(=O)C(C)(C)C)C4)cc3n12] | (7R,14R)-1-(difluoromethoxy)-6- (methyl-d₃)-11-((1-pivaloylazetidin-3- yl)ethynyl)-6,7-dihydro-7,14- methanobenzo[f]benzo[4,5]imidazo[1,2- a][1,4]diazocin-5(14H)-one |
| 126 | 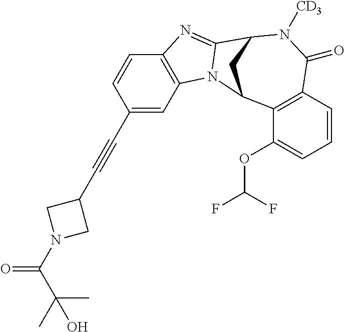 [SMILES: [2H]C([2H])([2H])N1C(=O)c2cccc(OC(F)F)c2[C@H]2C[C@@H]1c1nc3ccc(C#CC4CN(C(=O)C(C)(C)O)C4)cc3n12] | (7R,14R)-1-(difluoromethoxy)-11-((1-(2- hydroxy-2-methylpropanoyl)azetidin-3- yl)ethynyl)-6-(methyl-d₃)-6,7-dihydro- 7,14- methanobenzo[f]benzo[4,5]imidazo[1,2- a][1,4]diazocin-5(14H)-one |
| 127 | 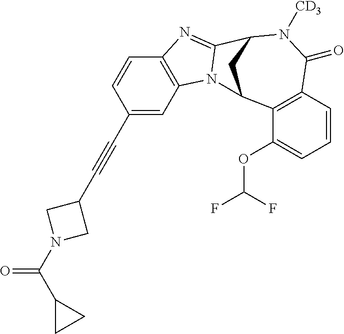 [SMILES: [2H]C([2H])([2H])N1C(=O)c2cccc(OC(F)F)c2[C@H]2C[C@@H]1c1nc3ccc(C#CC4CN(C(=O)C5CC5)C4)cc3n12] | (7R,14R)-11-((1- (cyclopropanecarbonyl)azetidin-3- yl)ethynyl)-1-(difluoromethoxy)-6- (methyl-d₃)-6,7-dihydro-7,14- methanobenzo[f]benzo[4,5]imidazo[1,2- a][1,4]diazocin-5(14H)-one |
| 128 | 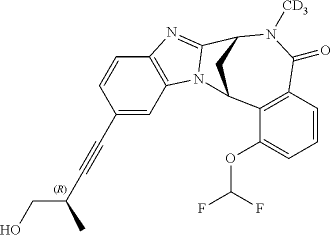 | (7R,14R)-1-(difluoromethoxy)-11-((R)- 4-hydroxy-3-methylbut-1-yn-1-yl)-6- (methyl-d₃)-6,7-dihydro-7,14- methanobenzo[f]benzo[4,5]imidazo[1,2- a][1,4]diazocin-5(14H)-one |
| 129 | 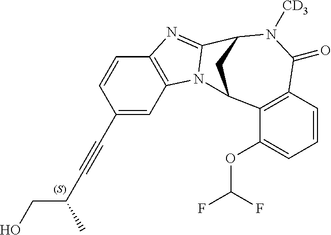 | (7R,14R)-1-(difluoromethoxy)-11-((S)-4- hydroxy-3-methylbut-1-yn-1-yl)-6- (methyl-d₃)-6,7-dihydro-7,14- methanobenzo[f]benzo[4,5]imidazo[1,2- a][1,4]diazocin-5(14H)-one |
| 130 | 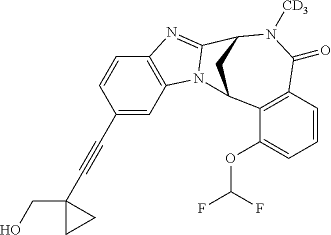 [SMILES: [2H]C([2H])([2H])N1C(=O)c2cccc(OC(F)F)c2[C@H]2C[C@@H]1c1nc3ccc(C#CC4(CO)CC4)cc3n12] | (7R,14R)-1-(difluoromethoxy)-11-((1- (hydroxymethyl)cyclopropyl)ethynyl)-6- (methyl-d₃)-6,7-dihydro-7,14- methanobenzo[f]benzo[4,5]imidazo[1,2- a][1,4]diazocin-5(14H)-one |
| 131 | 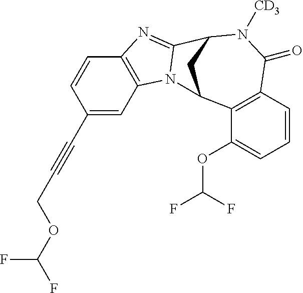 [SMILES: [2H]C([2H])([2H])N1C(=O)c2cccc(OC(F)F)c2[C@H]2C[C@@H]1c1nc3ccc(C#CCOC(F)F)cc3n12] | (7R,14R)-1-(difluoromethoxy)-11-(3- (difluoromethoxy)prop-1-yn-1-yl)-6- (methyl-d₃)-6,7-dihydro-7,14- methanobenzo[f]benzo[4,5]imidazo[1,2- a][1,4]diazocin-5(14H)-one |
| 132 | 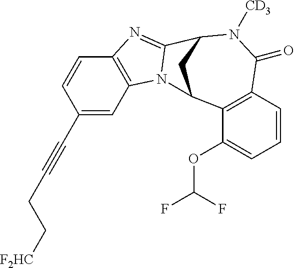 [SMILES: [2H]C([2H])([2H])N1C(=O)c2cccc(OC(F)F)c2[C@H]2C[C@@H]1c1nc3ccc(C#CCCC(F)F)cc3n12] | (7R,14R)-1-(difluoromethoxy)-11-(5,5- difluoropent-1-yn-1-yl)-6-(methyl-d₃)- 6,7-dihydro-7,14- methanobenzo[f]benzo[4,5]imidazo[1,2- a][1,4]diazocin-5(14H)-one |
| 133 | 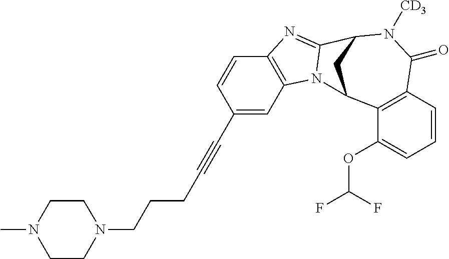 [SMILES: [2H]C([2H])([2H])N1C(=O)c2cccc(OC(F)F)c2[C@H]2C[C@@H]1c1nc3ccc(C#CCCCN4CCN(C)CC4)cc3n12] | (7R,14R)-1-(difluoromethoxy)-6- (methyl-d₃)-11-(5-(4-methylpiperazin-1- yl)pent-1-yn-1-yl)-6,7-dihydro-7,14- methanobenzo[f]benzo[4,5]imidazo[1,2- a][1,4]diazocin-5(14H)-one |
| 134 | 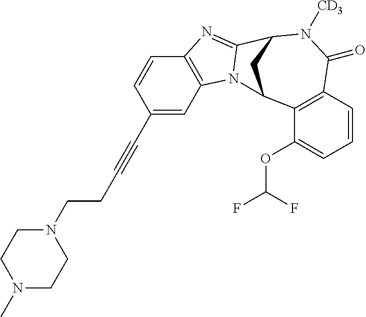 [SMILES: [2H]C([2H])([2H])N1C(=O)c2cccc(OC(F)F)c2[C@H]2C[C@@H]1c1nc3ccc(C#CCCN4CCN(C)CC4)cc3n12] | (7R,14R)-1-(difluoromethoxy)-6- (methyl-d₃)-11-(4-(4-methylpiperazin-1- yl)but-1-yn-1-yl)-6,7-dihydro-7,14- methanobenzo[f]benzo[4,5]imidazo[1,2- a][1,4]diazocin-5(14H)-one |
| 135 | 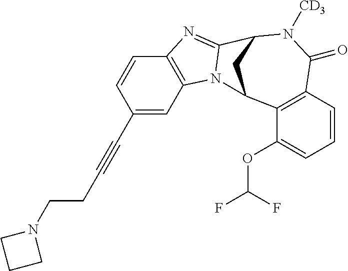 [SMILES: [2H]C([2H])([2H])N1C(=O)c2cccc(OC(F)F)c2[C@H]2C[C@@H]1c1nc3ccc(C#CCCN4CCC4)cc3n12] | (7R,14R)-11-(4-(azetidin-1-yl)but-1-yn- 1-yl)-1-(difluoromethoxy)-6-(methyl-d₃)- 6,7-dihydro-7,14- methanobenzo[f]benzo[4,5]imidazo[1,2- a][1,4]diazocin-5(14H)-one |
| 136 | 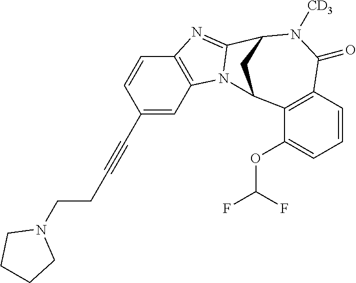 [SMILES: [2H]C([2H])([2H])N1C(=O)c2cccc(OC(F)F)c2[C@H]2C[C@@H]1c1nc3ccc(C#CCCN4CCCC4)cc3n12] | (7R,14R)-1-(difluoromethoxy)-6- (methyl-d₃)-11-(4-(pyrrolidin-1-yl)but-1- yn-1-yl)-6,7-dihydro-7,14- methanobenzo[f]benzo[4,5]imidazo[1,2- a][1,4]diazocin-5(14H)-one |
| 137 | 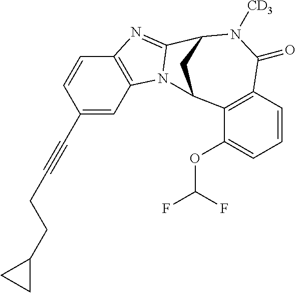 [SMILES: [2H]C([2H])([2H])N1C(=O)c2cccc(OC(F)F)c2[C@H]2C[C@@H]1c1nc3ccc(C#CCCC4CC4)cc3n12] | (7R,14R)-11-(4-cyclopropylbut-1-yn-1- yl)-1-(difluoromethoxy)-6-(methyl-d₃)- 6,7-dihydro-7,14- methanobenzo[f]benzo[4,5]imidazo[1,2- a][1,4]diazocin-5(14H)-one |
| 138 | 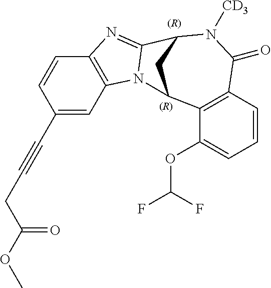 | methyl 4-((7R,14R)-1- (difluoromethoxy)-6-(methyl-d₃)-5-oxo- 5,6,7,14-tetrahydro-7,14- methanobenzo[f]benzo[4,5]imidazo[1,2- a][1,4]diazocin-11-yl)but-3-ynoate |
| 139 and 140 | 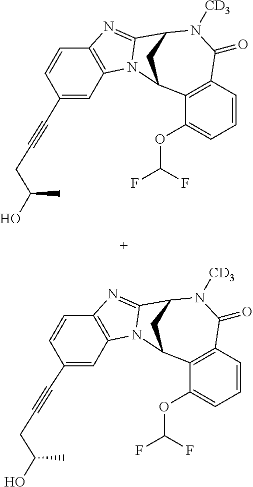 [SMILES: [2H]C([2H])([2H])N1C(=O)c2cccc(OC(F)F)c2[C@H]2C[C@@H]1c1nc3ccc(C#CC[C@@H](C)O)cc3n12 ; [2H]C([2H])([2H])N1C(=O)c2cccc(OC(F)F)c2[C@H]2C[C@@H]1c1nc3ccc(C#CC[C@H](C)O)cc3n12] | (7R,14R)-1-(difluoromethoxy)-11-((R or S)-4-hydroxypent-1-yn-1-yl)-6-(methyl- d3)-6,7-dihydro-7,14- methanobenzo[f]benzo[4,5]imidazo[1,2- a][1,4]diazocin-5(14H)-one And (7R,14R)-1-(difluoromethoxy)-11- ((S or R)-4-hydroxypent-1-yn-1-yl)-6- (methyl-d3)-6,7-dihydro-7,14- methanobenzo[f]benzo[4,5]imidazo[1,2- a][1,4]diazocin-5(14H)-one |
| 141 | 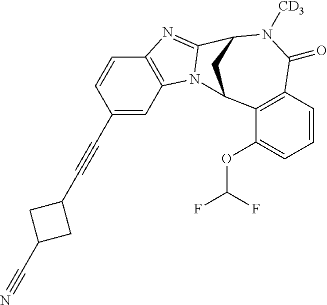 [SMILES: [2H]C([2H])([2H])N1C(=O)c2cccc(OC(F)F)c2[C@H]2C[C@@H]1c1nc3ccc(C#CC4CC(C#N)C4)cc3n12] | 3-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11- yl)ethynyl)cyclobutane-1-carbonitrile |
| 142 | 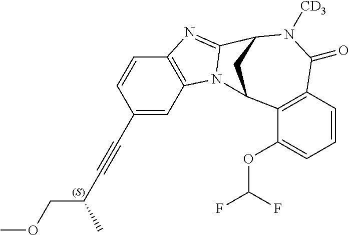 | (7R,14R)-1-(difluoromethoxy)-11-((S)-4- methoxy-3-methylbut-1-yn-1-yl)-6- (methyl-d3)-6,7-dihydro-7,14- methanobenzo[f]benzo[4,5]imidazo[1,2- a][1,4]diazocin-5(14H)-one |
| 143 | 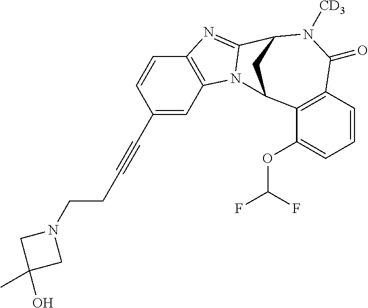 [SMILES: [2H]C([2H])([2H])N1C(=O)c2cccc(OC(F)F)c2[C@H]2C[C@@H]1c1nc3ccc(C#CCCN4CC(C)(O)C4)cc3n12] | (7R,14R)-1-(difluoromethoxy)-11-(4-(3- hydroxy-3-methylazetidin-1-yl)but-1-yn- 1-yl)-6-(methyl-d3)-6,7-dihydro-7,14- methanobenzo[f]benzo[4,5]imidazo[1,2- a][1,4]diazocin-5(14H)-one |
| 144 | 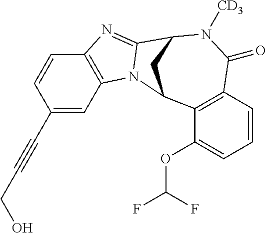 [SMILES: [2H]C([2H])([2H])N1C(=O)c2cccc(OC(F)F)c2[C@H]2C[C@@H]1c1nc3ccc(C#CCO)cc3n12] | (7R,14R)-1-(difluoromethoxy)-11-(3- hydroxyprop-1-yn-1-yl)-6-(methyl-d3)- 6,7-dihydro-7,14- methanobenzo[f]benzo[4,5]imidazo[1,2- a][1,4]diazocin-5(14H)-one |
| 145 | 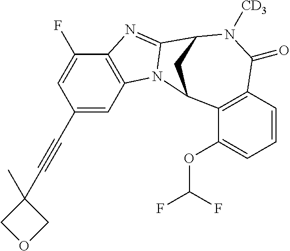 [SMILES: [2H]C([2H])([2H])N1C(=O)c2cccc(OC(F)F)c2[C@H]2C[C@@H]1c1nc3c(F)cc(C#CC4(C)COC4)cc3n12] | (7R,14R)-1-(difluoromethoxy)-9-fluoro- 6-(methyl-d3)-11-((3-methyloxetan-3- yl)ethynyl)-6,7-dihydro-7,14- methanobenzo[f]benzo[4,5]imidazo[1,2- a][1,4]diazocin-5(14H)-one |
| 146 | 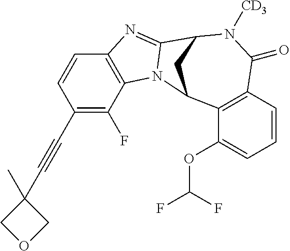 [SMILES: [2H]C([2H])([2H])N1C(=O)c2cccc(OC(F)F)c2[C@H]2C[C@@H]1c1nc3ccc(C#CC4(C)COC4)c(F)c3n12] | (7R,14R)-1-(difluoromethoxy)-12- fluoro-6-(methyl-d3)-11-((3- methyloxetan-3-yl)ethynyl)-6,7-dihydro- 7,14- methanobenzo[f]benzo[4,5]imidazo[1,2- a][1,4]diazocin-5(14H)-one |
| 147 | 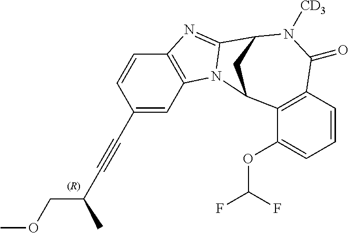 | (7R,14R)-1-(difluoromethoxy)-11-((R)- 4-methoxy-3-methylbut-1-yn-1-yl)-6- (methyl-d3)-6,7-dihydro-7,14- methanobenzo[f]benzo[4,5]imidazo[1,2- a][1,4]diazocin-5(14H)-one |
| 148 | 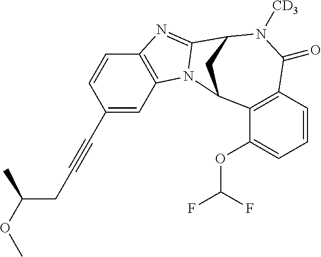 [SMILES: [2H]C([2H])([2H])N1C(=O)c2cccc(OC(F)F)c2[C@H]2C[C@@H]1c1nc3ccc(C#CC[C@H](C)OC)cc3n12] | (7R,14R)-1-(difluoromethoxy)-11-((R or S)-4-methoxypent-1-yn-1-yl)-6-(methyl- d3)-6,7-dihydro-7,14- methanobenzo[f]benzo[4,5]imidazo[1,2- a][1,4]diazocin-5(14H)-one |
| 149 | 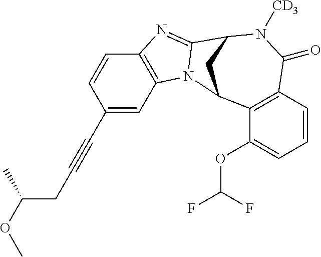 [SMILES: [2H]C([2H])([2H])N1C(=O)c2cccc(OC(F)F)c2[C@H]2C[C@@H]1c1nc3ccc(C#CC[C@@H](C)OC)cc3n12] | (7R,14R)-1-(difluoromethoxy)-11-((S or R)-4-methoxypent-1-yn-1-yl)-6-(methyl- d3)-6,7-dihydro-7,14- methanobenzo[f]benzo[4,5]imidazo[1,2- a][1,4]diazocin-5(14H)-one |
| 150 | 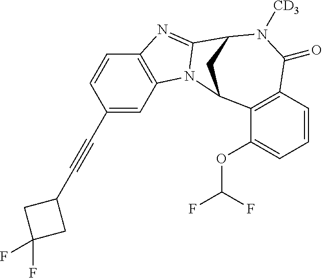 [SMILES: [2H]C([2H])([2H])N1C(=O)c2cccc(OC(F)F)c2[C@H]2C[C@@H]1c1nc3ccc(C#CC4CC(F)(F)C4)cc3n12] | (7R,14R)-11-((3,3- difluorocyclobutyl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 151 | 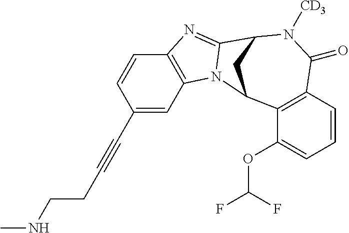 [SMILES: [2H]C([2H])([2H])N1C(=O)c2cccc(OC(F)F)c2[C@H]2C[C@@H]1c1nc3ccc(C#CCCNC)cc3n12] | (7R,14R)-1-(difluoromethoxy)-6- (methyl-d3)-11-(4-(methylamino)but-1- yn-1-yl)-6,7-dihydro-7,14- methanobenzo[f]benzo[4,5]imidazo[1,2- a][1,4]diazocin-5(14H)-one |
| 152 | 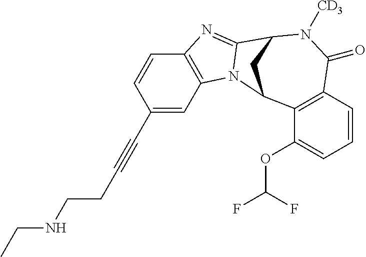 [SMILES: [2H]C([2H])([2H])N1C(=O)c2cccc(OC(F)F)c2[C@H]2C[C@@H]1c1nc3ccc(C#CCCNCC)cc3n12] | (7R,14R)-1-(difluoromethoxy)-11-(4- (ethylamino)but-1-yn-1-yl)-6-(methyl- d3)-6,7-dihydro-7,14- methanobenzo[f]benzo[4,5]imidazo[1,2- a][1,4]diazocin-5(14H)-one |
| 153 | 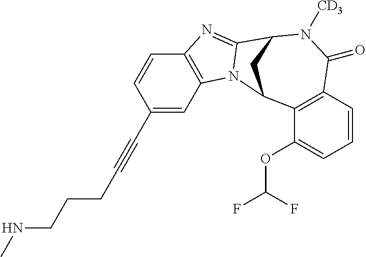 [SMILES: [2H]C([2H])([2H])N1C(=O)c2cccc(OC(F)F)c2[C@H]2C[C@@H]1c1nc3ccc(C#CCCCNC)cc3n12] | (7R,14R)-1-(difluoromethoxy)-6- (methyl-d3)-11-(5-(methylamino)pent-1- yn-1-yl)-6,7-dihydro-7,14- methanobenzo[f]benzo[4,5]imidazo[1,2- a][1,4]diazocin-5(14H)-one |
| 154 | 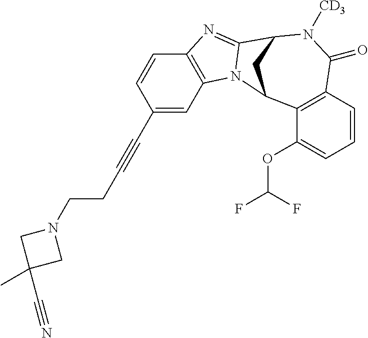 [SMILES: [2H]C([2H])([2H])N1C(=O)c2cccc(OC(F)F)c2[C@H]2C[C@@H]1c1nc3ccc(C#CCCN4CC(C)(C#N)C4)cc3n12] | 1-(4-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)but-3-yn-1-yl)-3- methylazetidine-3-carbonitrile |
| 155 | 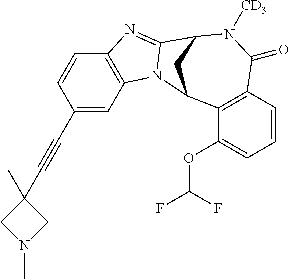 [SMILES: [2H]C([2H])([2H])N1C(=O)c2cccc(OC(F)F)c2[C@H]2C[C@@H]1c1nc3ccc(C#CC4(C)CN(C)C4)cc3n12] | (7R,14R)-1-(difluoromethoxy)-11-((1,3- dimethylazetidin-3-yl)ethynyl)-6- (methyl-d3)-6,7-dihydro-7,14- methanobenzo[f]benzo[4,5]imidazo[1,2- a][1,4]diazocin-5(14H)-one |
| 156 | 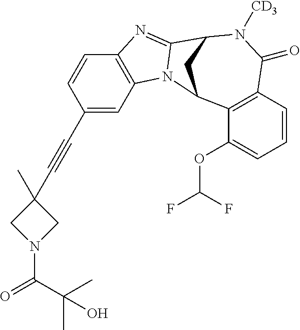 [SMILES: [2H]C([2H])([2H])N1C(=O)c2cccc(OC(F)F)c2[C@H]2C[C@@H]1c1nc3ccc(C#CC4(C)CN(C(=O)C(C)(C)O)C4)cc3n12] | (7R,14R)-1-(difluoromethoxy)-11-((1-(2- hydroxy-2-methylpropanoyl)-3- methylazetidin-3-yl)ethynyl)-6-(methyl- d3)-6,7-dihydro-7,14- methanobenzo[f]benzo[4,5]imidazo[1,2- a][1,4]diazocin-5(14H)-one |
| 157 | 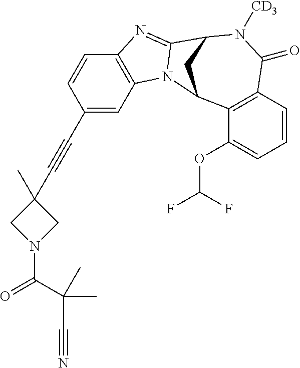 [SMILES: [2H]C([2H])([2H])N1C(=O)c2cccc(OC(F)F)c2[C@H]2C[C@@H]1c1nc3ccc(C#CC4(C)CN(C(=O)C(C)(C)C#N)C4)cc3n12] | 3-(3-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)-3- methylazetidin-1-yl)-2,2-dimethyl-3- oxopropanenitrile |
| 158 | 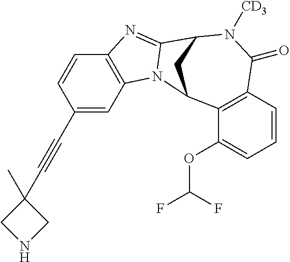 [SMILES: [2H]C([2H])([2H])N1C(=O)c2cccc(OC(F)F)c2[C@H]2C[C@@H]1c1nc3ccc(C#CC4(C)CNC4)cc3n12] | (7R,14R)-1-(difluoromethoxy)-6- (methyl-d3)-11-((3-methylazetidin-3- yl)ethynyl)-6,7-dihydro-7,14- methanobenzo[f]benzo[4,5]imidazo[1,2- a][1,4]diazocin-5(14H)-one |
| 159 | 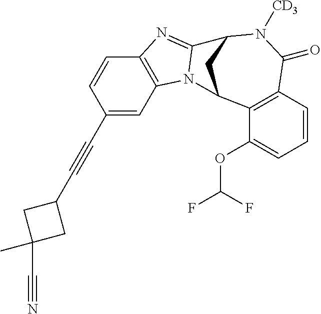 [SMILES: [2H]C([2H])([2H])N1C(=O)c2cccc(OC(F)F)c2[C@H]2C[C@@H]1c1nc3ccc(C#CC4CC(C)(C#N)C4)cc3n12] | 3-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)-1- methylcyclobutane-1-carbonitrile |
| 160 | 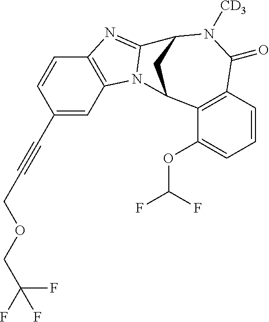 [SMILES: [2H]C([2H])([2H])N1C(=O)c2cccc(OC(F)F)c2[C@H]2C[C@@H]1c1nc3ccc(C#CCOCC(F)(F)F)cc3n12] | (7R,14R)-1-(difluoromethoxy)-6- (methyl-d3)-11-(3-(2,2,2- trifluoroethoxy)prop-1-yn-1-yl)-6,7- dihydro-7,14- methanobenzo[f]benzo[4,5]imidazo[1,2- a][1,4]diazocin-5(14H)-one |
| 161 | 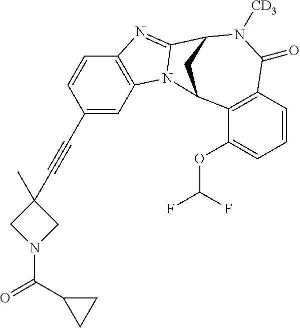 [SMILES: [2H]C([2H])([2H])N1C(=O)c2cccc(OC(F)F)c2[C@H]2C[C@@H]1c1nc3ccc(C#CC4(C)CN(C(=O)C5CC5)C4)cc3n12] | (7R,14R)-11-((1- (cyclopropanecarbonyl)-3- methylazetidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 162 | 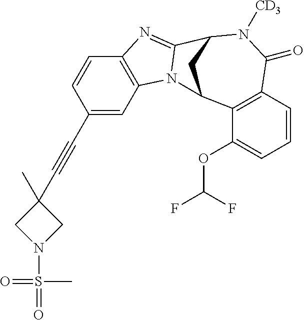 [SMILES: [2H]C([2H])([2H])N1C(=O)c2cccc(OC(F)F)c2[C@H]2C[C@@H]1c1nc3ccc(C#CC4(C)CN(S(C)(=O)=O)C4)cc3n12] | (7R,14R)-1-(difluoromethoxy)-6- (methyl-d3)-11-((3-methyl-1- (methylsulfonyl)azetidin-3-yl)ethynyl)- 6,7-dihydro-7,14- methanobenzo[f]benzo[4,5]imidazo[1,2- a][1,4]diazocin-5(14H)-one |
| 163 | 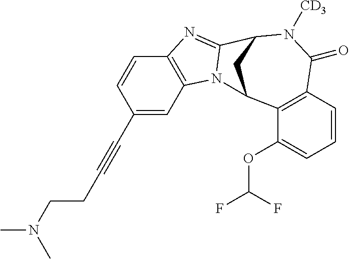 [SMILES: [2H]C([2H])([2H])N1C(=O)c2cccc(OC(F)F)c2[C@H]2C[C@@H]1c1nc3ccc(C#CCCN(C)C)cc3n12] | (7R,14R)-1-(difluoromethoxy)-11-(4- (dimethylamino)but-1-yn-1-yl)-6- (methyl-d3)-6,7-dihydro-7,14- methanobenzo[f]benzo[4,5]imidazo[1,2- a][1,4]diazocin-5(14H)-one |
| 164 | 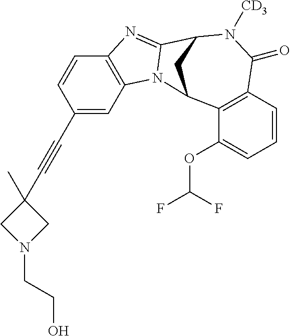 [SMILES: [2H]C([2H])([2H])N1C(=O)c2cccc(OC(F)F)c2[C@H]2C[C@@H]1c1nc3ccc(C#CC4(C)CN(CCO)C4)cc3n12] | (7R,14R)-1-(difluoromethoxy)-11-((1-(2- hydroxyethyl)-3-methylazetidin-3- yl)ethynyl)-6-(methyl-d3)-6,7-dihydro- 7,14- methanobenzo[f]benzo[4,5]imidazo[1,2- a][1,4]diazocin-5(14H)-one |
| 165 | 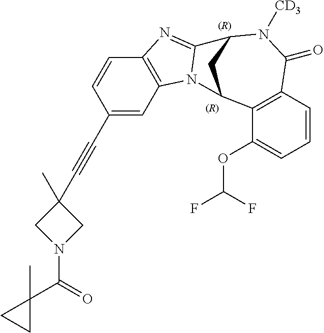 | (7R,14R)-1-(difluoromethoxy)-6- (methyl-d3)-11-((3-methyl-1-(1- methylcyclopropane-1-carbonyl)azetidin- 3-yl)ethynyl)-6,7-dihydro-7,14- methanobenzo[f]benzo[4,5]imidazo[1,2- a][1,4]diazocin-5(14H)-one |
| 166 | 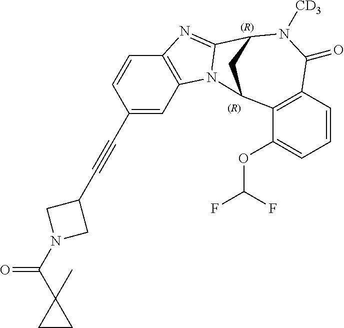 | (7R,14R)-1-(difluoromethoxy)-6- (methyl-d3)-11-((1-(1- methylcyclopropane-1-carbonyl)azetidin- 3-yl)ethynyl)-6,7-dihydro-7,14- methanobenzo[f]benzo[4,5]imidazo[1,2- a][1,4]diazocin-5(14H)-one |
| 167 | 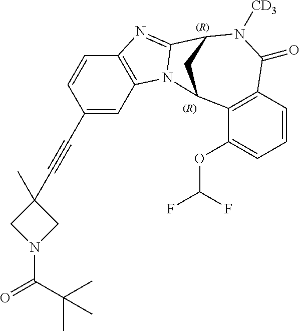 | (7R,14R)-1-(difluoromethoxy)-6- (methyl-d3)-11-((3-methyl-1- pivaloylazetidin-3-yl)ethynyl)-6,7- dihydro-7,14- methanobenzo[f]benzo[4,5]imidazo[1,2- a][1,4]diazocin-5(14H)-one |
| 168 | 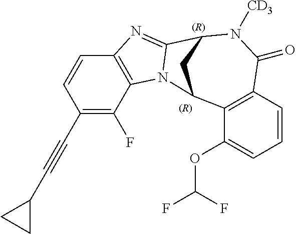 | (7R,14R)-11-(cyclopropylethynyl)-1- (difluoromethoxy)-12-fluoro-6-(methyl- d3)-6,7-dihydro-7,14- methanobenzo[f]benzo[4,5]imidazo[1,2- a][1,4]diazocin-5(14H)-one |
| 169 | 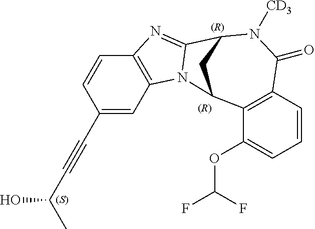 | (7R,14R)-1-(difluoromethoxy)-11-((S)-3- hydroxybut-1-yn-1-yl)-6-(methyl-d3)- 6,7-dihydro-7,14- methanobenzo[f]benzo[4,5]imidazo[1,2- a][1,4]diazocin-5(14H)-one |
| 170 | 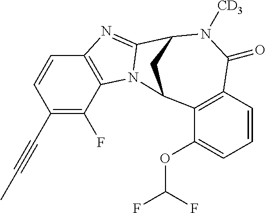 [SMILES: [2H]C([2H])([2H])N1C(=O)c2cccc(OC(F)F)c2[C@H]2C[C@@H]1c1nc3ccc(C#CC)c(F)c3n12] | (7R,14R)-1-(difluoromethoxy)-12- fluoro-6-(methyl-d3)-11-(prop-1-yn-1- yl)-6,7-dihydro-7,14- methanobenzo[f]benzo[4,5]imidazo[1,2- a][1,4]diazocin-5(14H)-one |
| 171 | 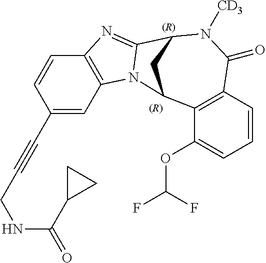 | N-(3-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)prop-2-yn-1- yl)cyclopropanecarboxamide |
| 172 | 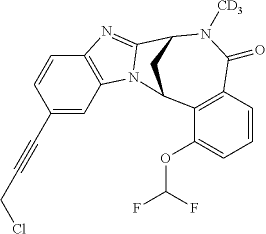 [SMILES: [2H]C([2H])([2H])N1C(=O)c2cccc(OC(F)F)c2[C@H]2C[C@@H]1c1nc3ccc(C#CCCl)cc3n12] | (7R,14R)-11-(3-chloroprop-1-yn-1-yl)-1- (difluoromethoxy)-6-(methyl-d3)-6,7- dihydro-7,14- methanobenzo[f]benzo[4,5]imidazo[1,2- a][1,4]diazocin-5(14H)-one |
| 173 | 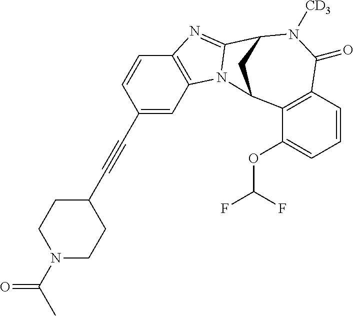 [SMILES: [2H]C([2H])([2H])N1C(=O)c2cccc(OC(F)F)c2[C@H]2C[C@@H]1c1nc3ccc(C#CC4CCN(C(C)=O)CC4)cc3n12] | (7R,14R)-11-((1-acetylpiperidin-4- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 174 | 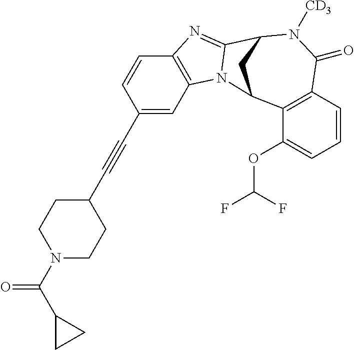 [SMILES: [2H]C([2H])([2H])N1C(=O)c2cccc(OC(F)F)c2[C@H]2C[C@@H]1c1nc3ccc(C#CC4CCN(C(=O)C5CC5)CC4)cc3n12] | (7R,14R)-11-((1- (cyclopropanecarbonyl)piperidin-4- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 175 | 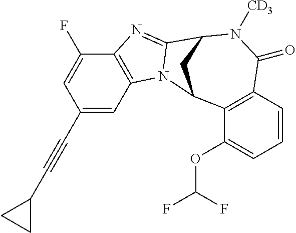 [SMILES: [2H]C([2H])([2H])N1C(=O)c2cccc(OC(F)F)c2[C@H]2C[C@@H]1c1nc3c(F)cc(C#CC4CC4)cc3n12] | (7R,14R)-11-(cyclopropylethynyl)-1- (difluoromethoxy)-9-fluoro-6-(methyl- d3)-6,7-dihydro-7,14- methanobenzo[f]benzo[4,5]imidazo[1,2- a][1,4]diazocin-5(14H)-one |
| 176 | 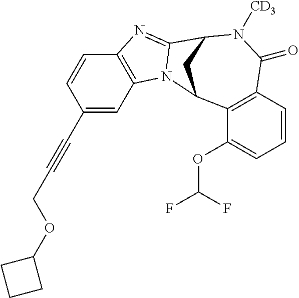 [SMILES: [2H]C([2H])([2H])N1C(=O)c2cccc(OC(F)F)c2[C@H]2C[C@@H]1c1nc3ccc(C#CCOC4CCC4)cc3n12] | (7R,14R)-11-(3-cyclobutoxyprop-1-yn-1- yl)-1-(difluoromethoxy)-6-(methyl-d3)- 6,7-dihydro-7,14- methanobenzo[f]benzo[4,5]imidazo[1,2- a][1,4]diazocin-5(14H)-one |
| 177 | 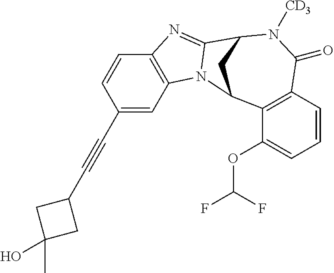 [SMILES: [2H]C([2H])([2H])N1C(=O)c2cccc(OC(F)F)c2[C@H]2C[C@@H]1c1nc3ccc(C#CC4CC(C)(O)C4)cc3n12] | (7R,14R)-1-(difluoromethoxy)-11-((3- hydroxy-3-methylcyclobutyl)ethynyl)-6- (methyl-d3)-6,7-dihydro-7,14- methanobenzo[f]benzo[4,5]imidazo[1,2- a][1,4]diazocin-5(14H)-one |
| 178 | 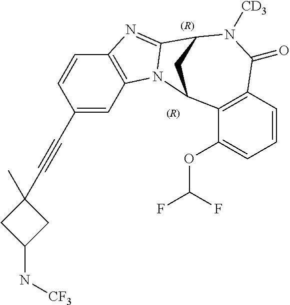 | (7R,14R)-1-(difluoromethoxy)-6- (methyl-d3)-11-((3-methyl-1-(2,2,2- trifluoroethyl)azetidin-3-yl)ethynyl)-6,7- dihydro-7,14- methanobenzo[f]benzo[4,5]imidazo[1,2- a][1,4]diazocin-5(14H)-one |
| 179 | 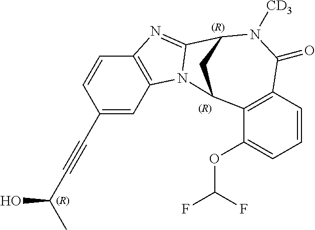 | (7R,14R)-1-(difluoromethoxy)-11-((R)- 3-hydroxybut-1-yn-1-yl)-6-(methyl-d3)- 6,7-dihydro-7,14- methanobenzo[f]benzo[4,5]imidazo[1,2- a][1,4]diazocin-5(14H)-one |
| 180 | 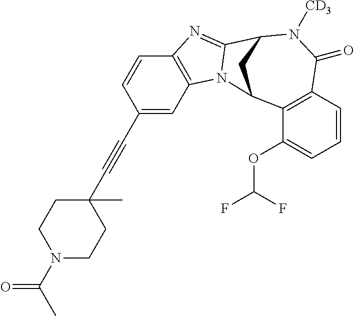 [SMILES: [2H]C([2H])([2H])N1C(=O)c2cccc(OC(F)F)c2[C@H]2C[C@@H]1c1nc3ccc(C#CC4(C)CCN(C(C)=O)CC4)cc3n12] | (7R,14R)-11-((1-acetyl-4- methylpiperidin-4-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 181 | 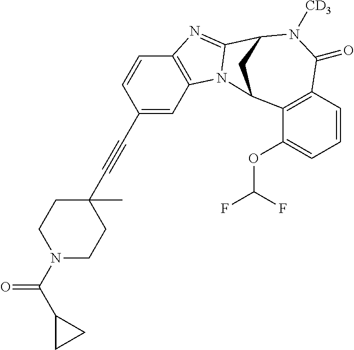 [SMILES: [2H]C([2H])([2H])N1C(=O)c2cccc(OC(F)F)c2[C@H]2C[C@@H]1c1nc3ccc(C#CC4(C)CCN(C(=O)C5CC5)CC4)cc3n12] | (7R,14R)-11-((1- (cyclopropanecarbonyl)-4- methylpiperidin-4-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 182 | 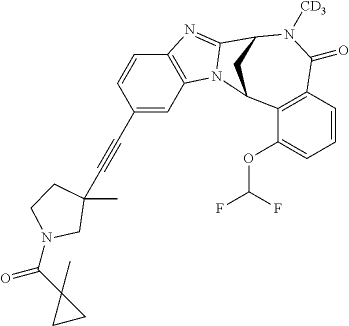 [SMILES: [2H]C([2H])([2H])N1C(=O)c2cccc(OC(F)F)c2[C@H]2C[C@@H]1c1nc3ccc(C#CC4(C)CCN(C(=O)C5(C)CC5)C4)cc3n12] | (7R,14R)-1-(difluoromethoxy)-6- (methyl-d₃)-11-((3-methyl-1-(1- methylcyclopropane-1- carbonyl)pyrrolidin-3-yl)ethynyl)-6,7- dihydro-7,14- methanobenzo[f]benzo[4,5]imidazo[1,2- a][1,4]diazocin-5(14H)-one |
| 183 | 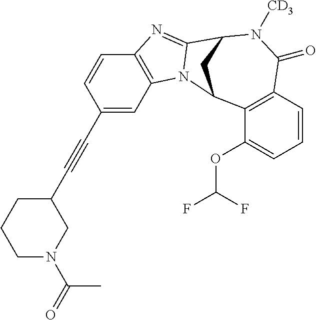 [SMILES: [2H]C([2H])([2H])N1C(=O)c2cccc(OC(F)F)c2[C@H]2C[C@@H]1c1nc3ccc(C#CC4CCCN(C(C)=O)C4)cc3n12] | (7R,14R)-11-((1-acetylpiperidin-3- yl)ethynyl)-1-(difluoromethoxy)-6- (methyl-d₃)-6,7-dihydro-7,14- methanobenzo[f]benzo[4,5]imidazo[1,2- a][1,4]diazocin-5(14H)-one |
| 184 | 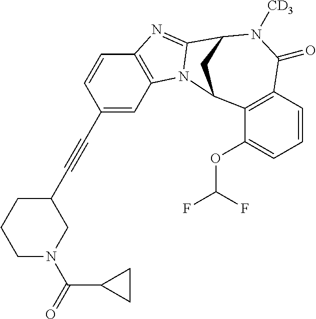 [SMILES: [2H]C([2H])([2H])N1C(=O)c2cccc(OC(F)F)c2[C@H]2C[C@@H]1c1nc3ccc(C#CC4CCCN(C(=O)C5CC5)C4)cc3n12] | (7R,14R)-11-((1- (cyclopropanecarbonyl)piperidin-3- yl)ethynyl)-1-(difluoromethoxy)-6- (methyl-d₃)-6,7-dihydro-7,14- methanobenzo[f]benzo[4,5]imidazo[1,2- a][1,4]diazocin-5(14H)-one |
| 185 | 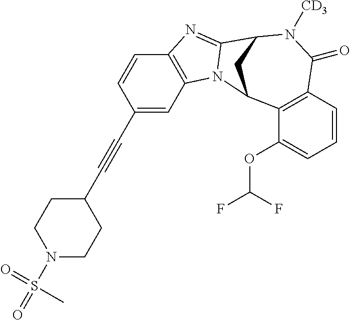 [SMILES: [2H]C([2H])([2H])N1C(=O)c2cccc(OC(F)F)c2[C@H]2C[C@@H]1c1nc3ccc(C#CC4CCN(S(C)(=O)=O)CC4)cc3n12] | (7R,14R)-1-(difluoromethoxy)-6- (methyl-d3)-11-((1- (methylsulfonyl)piperidin-4-yl)ethynyl)- 6,7-dihydro-7,14- methanobenzo[f]benzo[4,5]imidazo[1,2- a][1,4]diazocin-5(14H)-one |
| 186 | 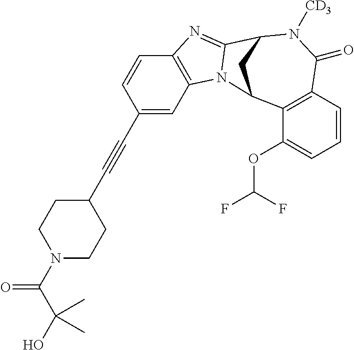 [SMILES: [2H]C([2H])([2H])N1C(=O)c2cccc(OC(F)F)c2[C@H]2C[C@@H]1c1nc3ccc(C#CC4CCN(C(=O)C(C)(C)O)CC4)cc3n12] | (7R,14R)-1-(difluoromethoxy)-11-((1-(2- hydroxy-2-methylpropanoyl)piperidin-4- yl)ethynyl)-6-(methyl-d3)-6,7-dihydro- 7,14- methanobenzo[f]benzo[4,5]imidazo[1,2- a][1,4]diazocin-5(14H)-one |
| 187 | 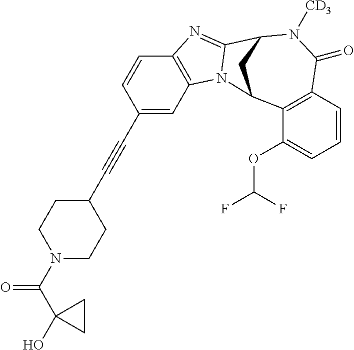 [SMILES: [2H]C([2H])([2H])N1C(=O)c2cccc(OC(F)F)c2[C@H]2C[C@@H]1c1nc3ccc(C#CC4CCN(C(=O)C5(O)CC5)CC4)cc3n12] | (7R,14R)-1-(difluoromethoxy)-11-((1-(1- hydroxycyclopropane-1- carbonyl)piperidin-4-yl)ethynyl)-6- (methyl-d3)-6,7-dihydro-7,14- methanobenzo[f]benzo[4,5]imidazo[1,2- a][1,4]diazocin-5(14H)-one |
| 188 | 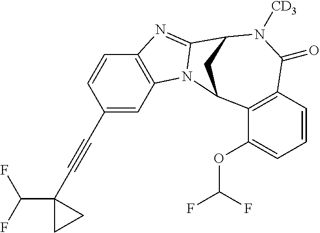 [SMILES: [2H]C([2H])([2H])N1C(=O)c2cccc(OC(F)F)c2[C@H]2C[C@@H]1c1nc3ccc(C#CC4(C(F)F)CC4)cc3n12] | (7R,14R)-1-(difluoromethoxy)-11-((1- (difluoromethyl)cyclopropyl)ethynyl)-6- (methyl-d3)-6,7-dihydro-7,14- methanobenzo[f]benzo[4,5]imidazo[1,2- a][1,4]diazocin-5(14H)-one |

| Synthetic | | |
|---|---|---|
| Chemistry | | |
| Example | Compound Structure | Compound Name |
| 189 | 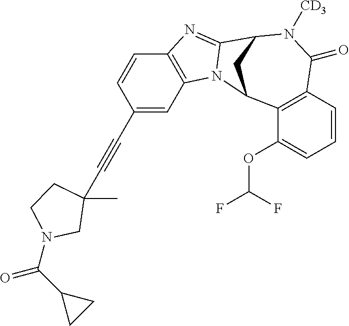 [SMILES: [2H]C([2H])([2H])N1C(=O)c2cccc(OC(F)F)c2[C@H]2C[C@@H]1c1nc3ccc(C#CC4(C)CCN(C(=O)C5CC5)C4)cc3n12] | (7R,14R)-11-((1-(cyclopropanecarbonyl)- 3-methylpyrrolidin-3-yl)ethynyl)-1- (difluoromethoxy)-6-(methyl-d₃)-6,7- dihydro-7,14- methanobenzo[f]benzo[4,5]imidazo[1,2- a][1,4]diazocin-5(14H)-one |
| 190 | 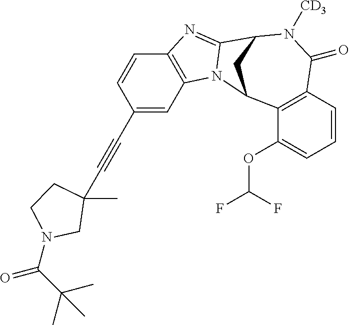 [SMILES: [2H]C([2H])([2H])N1C(=O)c2cccc(OC(F)F)c2[C@H]2C[C@@H]1c1nc3ccc(C#CC4(C)CCN(C(=O)C(C)(C)C)C4)cc3n12] | (7R,14R)-1-(difluoromethoxy)-6- (methyl-d₃)-11-((3-methyl-1- pivaloylpyrrolidin-3-yl)ethynyl)-6,7- dihydro-7,14- methanobenzo[f]benzo[4,5]imidazo[1,2- a][1,4]diazocin-5(14H)-one |
| 191 | 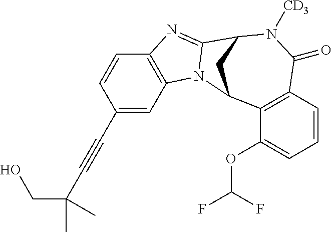 [SMILES: [2H]C([2H])([2H])N1C(=O)c2cccc(OC(F)F)c2[C@H]2C[C@@H]1c1nc3ccc(C#CC(C)(C)CO)cc3n12] | (7R,14R)-1-(difluoromethoxy)-11-(4- hydroxy-3,3-dimethylbut-1-yn-1-yl)-6- (methyl-d3)-6,7-dihydro-7,14- methanobenzo[f]benzo[4,5]imidazo[1,2- a][1,4]diazocin-5(14H)-one |
| 192 | 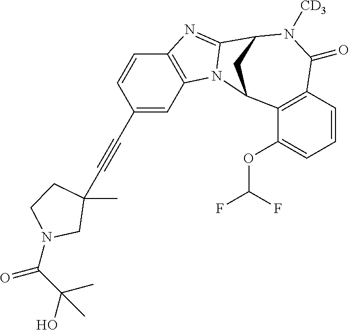 [SMILES: [2H]C([2H])([2H])N1C(=O)c2cccc(OC(F)F)c2[C@H]2C[C@@H]1c1nc3ccc(C#CC4(C)CCN(C(=O)C(C)(C)O)C4)cc3n12] | (7R,14R)-1-(difluoromethoxy)-11-((1-(2- hydroxy-2-methylpropanoyl)-3- methylpyrrolidin-3-yl)ethynyl)-6- (methyl-d₃)-6,7-dihydro-7,14- methanobenzo[f]benzo[4,5]imidazo[1,2- a][1,4]diazocin-5(14H)-one |
| 193 | 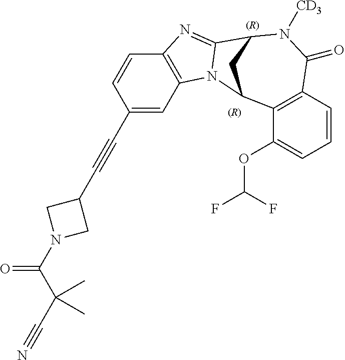 | 3-(3-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)azetidin-1- yl)-2,2-dimethyl-3-oxopropanenitrile |
| 194 | 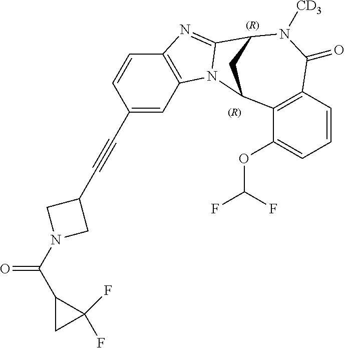 | (7R,14R)-11-((1-(2,2- difluorocyclopropane-1- carbonyl)azetidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 195 | 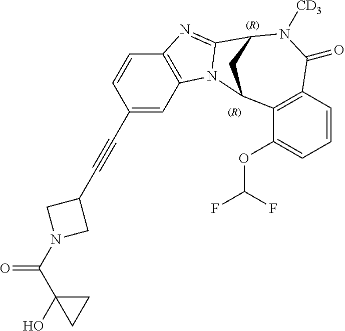 | (7R,14R)-1-(difluoromethoxy)-11-((1-(1- hydroxycyclopropane-1- carbonyl)azetidin-3-yl)ethynyl)-6- (methyl-d3)-6,7-dihydro-7,14- methanobenzo[f]benzo[4,5]imidazo[1,2- a][1,4]diazocin-5(14H)-one |
| 196 | 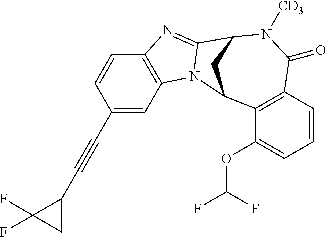 [SMILES: [2H]C([2H])([2H])N1C(=O)c2cccc(OC(F)F)c2[C@H]2C[C@@H]1c1nc3ccc(C#CC4CC4(F)F)cc3n12] | (7R,14R)-11-((2,2- difluorocyclopropyl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 197 | 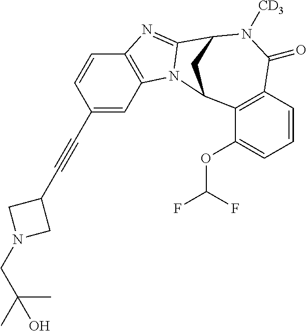 [SMILES: [2H]C([2H])([2H])N1C(=O)c2cccc(OC(F)F)c2[C@H]2C[C@@H]1c1nc3ccc(C#CC4CN(CC(C)(C)O)C4)cc3n12] | (7R,14R)-1-(difluoromethoxy)-11-((1-(2- hydroxy-2-methylpropyl)azetidin-3- yl)ethynyl)-6-(methyl-d3)-6,7-dihydro- 7,14- methanobenzo[f]benzo[4,5]imidazo[1,2- a][1,4]diazocin-5(14H)-one |
| 198 | 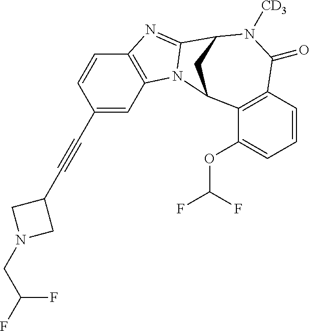 [SMILES: [2H]C([2H])([2H])N1C(=O)c2cccc(OC(F)F)c2[C@H]2C[C@@H]1c1nc3ccc(C#CC4CN(CC(F)F)C4)cc3n12] | (7R,14R)-11-((1-(2,2- difluoroethyl)azetidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 199 and 200 | 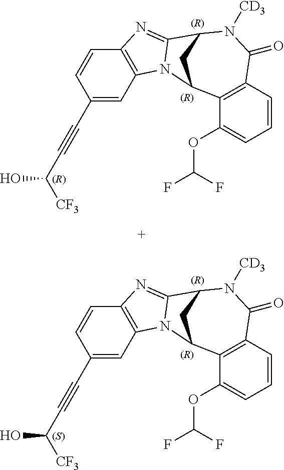 | (7R,14R)-1-(difluoromethoxy)-6- (methyl-d3)-11-((R or S)-4,4,4-trifluoro- 3-hydroxybut-1-yn-1-yl)-6,7-dihydro- 7,14- methanobenzo[f]benzo[4,5]imidazo[1,2- a][1,4]diazocin-5(14H)-one And (7R,14R)-1-(difluoromethoxy)-6- (methyl-d3)-11-((S or R)-4,4,4-trifluoro- 3-hydroxybut-1-yn-1-yl)-6,7-dihydro- 7,14- methanobenzo[f]benzo[4,5]imidazo[1,2- a][1,4]diazocin-5(14H)-one |
| 201 | 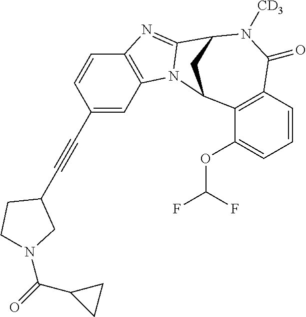 [SMILES: [2H]C([2H])([2H])N1C(=O)c2cccc(OC(F)F)c2[C@H]2C[C@@H]1c1nc3ccc(C#CC4CCN(C(=O)C5CC5)C4)cc3n12] | (7R,14R)-11-((1- (cyclopropanecarbonyl)pyrrolidin-3- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 202 | 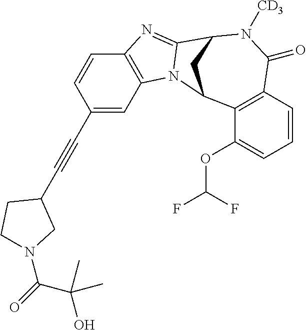 [SMILES: [2H]C([2H])([2H])N1C(=O)c2cccc(OC(F)F)c2[C@H]2C[C@@H]1c1nc3ccc(C#CC4CCN(C(=O)C(C)(C)O)C4)cc3n12] | (7R,14R)-1-(difluoromethoxy)-11-((1-(2- hydroxy-2-methylpropanoyl)pyrrolidin- 3-yl)ethynyl)-6-(methyl-d3)-6,7-dihydro- 7,14- methanobenzo[f]benzo[4,5]imidazo[1,2- a][1,4]diazocin-5(14H)-one |
| 203 | 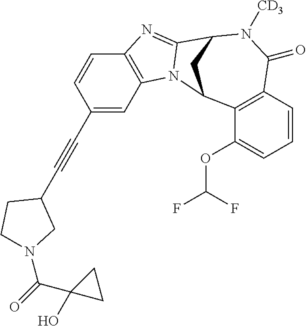 [SMILES: [2H]C([2H])([2H])N1C(=O)c2cccc(OC(F)F)c2[C@H]2C[C@@H]1c1nc3ccc(C#CC4CCN(C(=O)C5(O)CC5)C4)cc3n12] | (7R,14R)-1-(difluoromethoxy)-11-((1-(1- hydroxycyclopropane-1- carbonyl)pyrrolidin-3-yl)ethynyl)-6- (methyl-d3)-6,7-dihydro-7,14- methanobenzo[f]benzo[4,5]imidazo[1,2- a][1,4]diazocin-5(14H)-one |
| 204 | 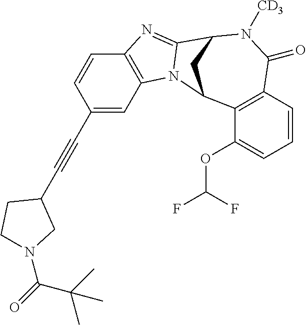 [SMILES: [2H]C([2H])([2H])N1C(=O)c2cccc(OC(F)F)c2[C@H]2C[C@@H]1c1nc3ccc(C#CC4CCN(C(=O)C(C)(C)C)C4)cc3n12] | (7R,14R)-1-(difluoromethoxy)-6- (methyl-d3)-11-((1-pivaloylpyrrolidin-3- yl)ethynyl)-6,7-dihydro-7,14- methanobenzo[f]benzo[4,5]imidazo[1,2- a][1,4]diazocin-5(14H)-one |
| 205 | 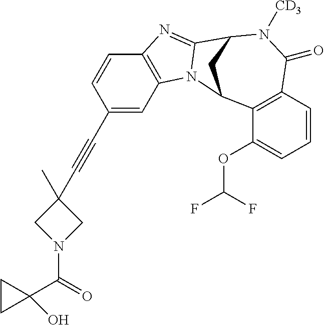 [SMILES: [2H]C([2H])([2H])N1C(=O)c2cccc(OC(F)F)c2[C@H]2C[C@@H]1c1nc3ccc(C#CC4(C)CN(C(=O)C5(O)CC5)C4)cc3n12] | (7R,14R)-1-(difluoromethoxy)-11-((1-(1- hydroxycyclopropane-1-carbonyl)-3- methylazetidin-3-yl)ethynyl)-6-(methyl- d3)-6,7-dihydro-7,14- methanobenzo[f]benzo[4,5]imidazo[1,2- a][1,4]diazocin-5(14H)-one |
| 206 | 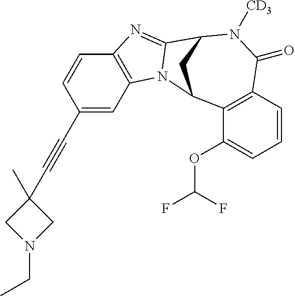 [SMILES: [2H]C([2H])([2H])N1C(=O)c2cccc(OC(F)F)c2[C@H]2C[C@@H]1c1nc3ccc(C#CC4(C)CN(CC)C4)cc3n12] | (7R,14R)-1-(difluoromethoxy)-11-((1- ethyl-3-methylazetidin-3-yl)ethynyl)-6- (methyl-d3)-6,7-dihydro-7,14- methanobenzo[f]benzo[4,5]imidazo[1,2- a][1,4]diazocin-5(14H)-one |
| 207 | 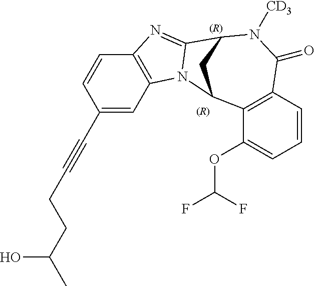 | (7R,14R)-1-(difluoromethoxy)-11-(5- hydroxyhex-1-yn-1-yl)-6-(methyl-d3)- 6,7-dihydro-7,14- methanobenzo[f]benzo[4,5]imidazo[1,2- a][1,4]diazocin-5(14H)-one |
| 208 and 209 | 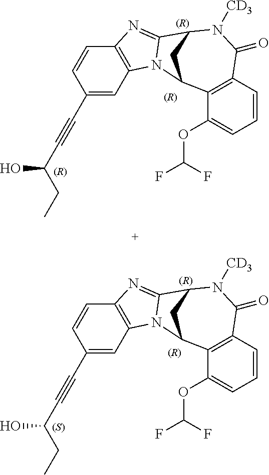 | (7R,14R)-1-(difluoromethoxy)-11-((R or S)-3-hydroxypent-1-yn-1-yl)-6-(methyl- d3)-6,7-dihydro-7,14- methanobenzo[f]benzo[4,5]imidazo[1,2- a][1,4]diazocin-5(14H)-one And (7R,14R)-1-(difluoromethoxy)-11- ((S or R)-3-hydroxypent-1-yn-1-yl)-6- (methyl-d3)-6,7-dihydro-7,14- methanobenzo[f]benzo[4,5]imidazo[1,2- a][1,4]diazocin-5(14H)-one |
| 210 | 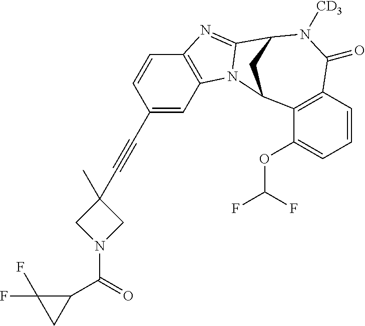 [SMILES: [2H]C([2H])([2H])N1C(=O)c2cccc(OC(F)F)c2[C@H]2C[C@@H]1c1nc3ccc(C#CC4(C)CN(C(=O)C5CC5(F)F)C4)cc3n12] | (7R,14R)-11-((1-(2,2- difluorocyclopropane-1-carbonyl)-3- methylazetidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 211 | 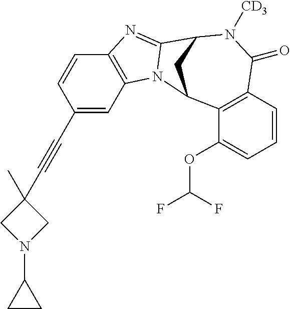 [SMILES: [2H]C([2H])([2H])N1C(=O)c2cccc(OC(F)F)c2[C@H]2C[C@@H]1c1nc3ccc(C#CC4(C)CN(C5CC5)C4)cc3n12] | (7R,14R)-11-((1-cyclopropyl-3- methylazetidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 212 | 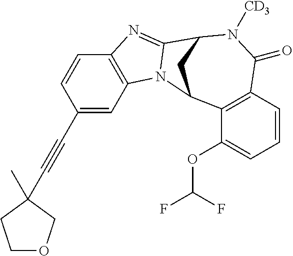 [SMILES: [2H]C([2H])([2H])N1C(=O)c2cccc(OC(F)F)c2[C@H]2C[C@@H]1c1nc3ccc(C#CC4(C)CCOC4)cc3n12] | (7R,14R)-1-(difluoromethoxy)-6- (methyl-d3)-11-((3- methyltetrahydrofuran-3-yl)ethynyl)-6,7- dihydro-7,14- methanobenzo[f]benzo[4,5]imidazo[1,2- a][1,4]diazocin-5(14H)-one |
| 213 | 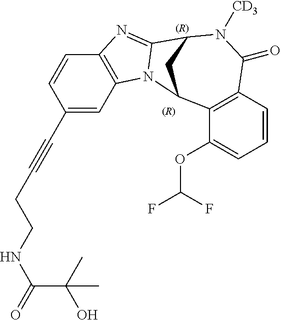 | N-(4-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)but-3-yn-1-yl)-2- hydroxy-2-methylpropanamide |
| 214 | 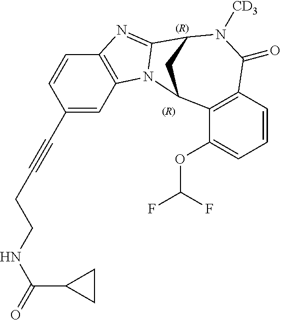 | N-(4-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)but-3-yn-1- yl)cyclopropanecarboxamide |
| 215 | 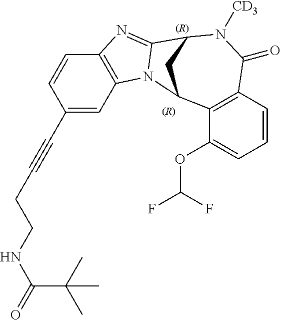 | N-(4-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)but-3-yn-1- yl)pivalamide |
| 216 and 217 | 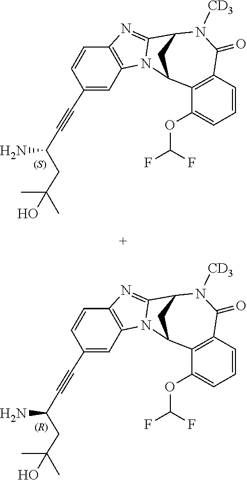 | (7R,14R)-11-((S or R)-3-amino-5- hydroxy-5-methylhex-1-yn-1-yl)-1- (difluoromethoxy)-6-(methyl-d3)-6,7- dihydro-7,14- methanobenzo[f]benzo[4,5]imidazo[1,2- a][1,4]diazocin-5(14H)-one And (7R,14R)-11-((R or S)-3-amino-5- hydroxy-5-methylhex-1-yn-1-yl)-1- (difluoromethoxy)-6-(methyl-d3)-6,7- dihydro-7,14- methanobenzo[f]benzo[4,5]imidazo[1,2- a][1,4]diazocin-5(14H)-one |
| 218 | 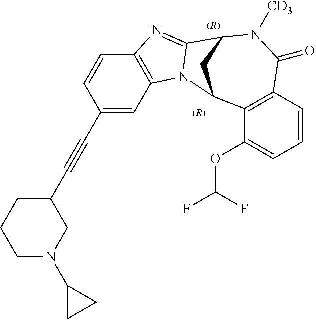 | (7R,14R)-11-((1-cyclopropylpiperidin-3- yl)ethynyl)-1-(difluoromethoxy)-6- (methyl-d₃)-6,7-dihydro-7,14- methanobenzo[f]benzo[4,5]imidazo[1,2- a][1,4]diazocin-5(14H)-one |
| 219 | 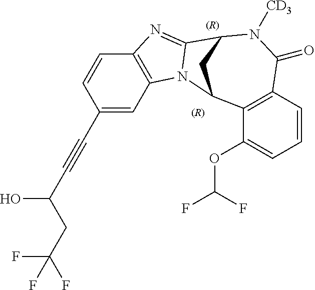 | (7R,14R)-1-(difluoromethoxy)-6- (methyl-d3)-11-(5,5,5-trifluoro-3- hydroxypent-1-yn-1-yl)-6,7-dihydro- 7,14- methanobenzo[f]benzo[4,5]imidazo[1,2- a][1,4]diazocin-5(14H)-one |
| 220 | 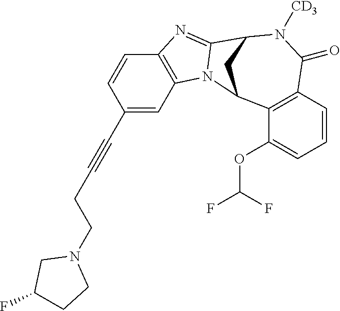 [SMILES: [2H]C([2H])([2H])N1C(=O)c2cccc(OC(F)F)c2[C@H]2C[C@@H]1c1nc3ccc(C#CCCN4CC[C@H](F)C4)cc3n12] | (7R,14R)-1-(difluoromethoxy)-11-(4- ((S)-3-fluoropyrrolidin-1-yl)but-1-yn-1- yl)-6-(methyl-d3)-6,7-dihydro-7,14- methanobenzo[f]benzo[4,5]imidazo[1,2- a][1,4]diazocin-5(14H)-one |
| 221 and 222 | 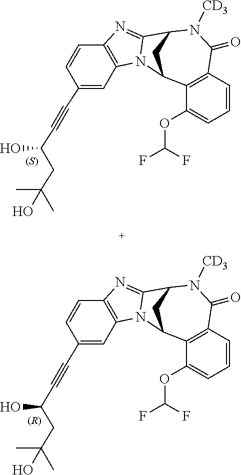 | (7R,14R)-1-(difluoromethoxy)-11-((S or R)-3,5-dihydroxy-5-methylhex-1-yn-1- yl)-6-(methyl-d3)-6,7-dihydro-7,14- methanobenzo[f]benzo[4,5]imidazo[1,2- a][1,4]diazocin-5(14H)-one And (7R,14R)-1-(difluoromethoxy)-11- ((R or S)-3,5-dihydroxy-5-methylhex-1- yn-1-yl)-6-(methyl-d3)-6,7-dihydro-7,14- methanobenzo[f]benzo[4,5]imidazo[1,2- a][1,4]diazocin-5(14H)-one |
| 223 | 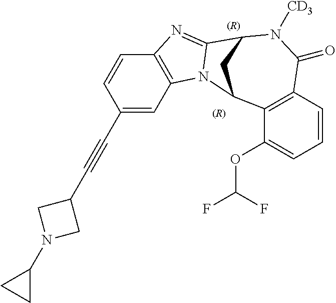 | (7R,14R)-11-((1-cyclopropylazetidin-3- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 224 | 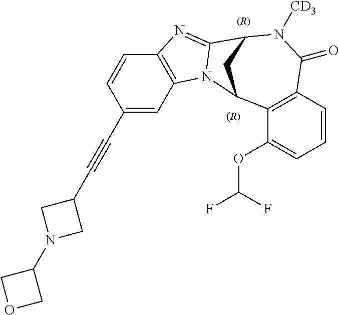 | (7R,14R)-1-(difluoromethoxy)-6- (methyl-d3)-11-((1-(oxetan-3-yl)azetidin- 3-yl)ethynyl)-6,7-dihydro-7,14- methanobenzo[f]benzo[4,5]imidazo[1,2- a][1,4]diazocin-5(14H)-one |
| 225 | 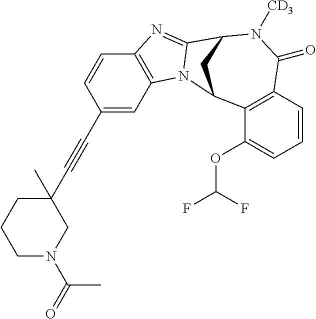 [SMILES: [2H]C([2H])([2H])N1C(=O)c2cccc(OC(F)F)c2[C@H]2C[C@@H]1c1nc3ccc(C#CC4(C)CCCN(C(C)=O)C4)cc3n12] | (7R,14R)-11-((1-acetyl-3- methylpiperidin-3-yl)ethynyl)-1- (difluoromethoxy)-6-(methyl-d3)-6,7- dihydro-7,14- methanobenzo[f]benzo[4,5]imidazo[1,2- a][1,4]diazocin-5(14H)-one |
| 226 | 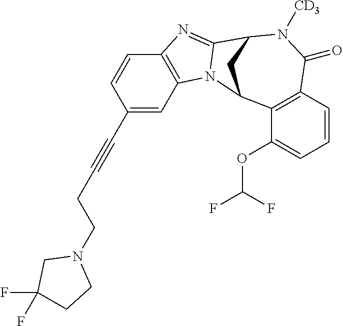 [SMILES: [2H]C([2H])([2H])N1C(=O)c2cccc(OC(F)F)c2[C@H]2C[C@@H]1c1nc3ccc(C#CCCN4CCC(F)(F)C4)cc3n12] | (7R,14R)-1-(difluoromethoxy)-11-(4- (3,3-difluoropyrrolidin-1-yl)but-1-yn-1- yl)-6-(methyl-d3)-6,7-dihydro-7,14- methanobenzo[f]benzo[4,5]imidazo[1,2- a][1,4]diazocin-5(14H)-one |
| 227 | 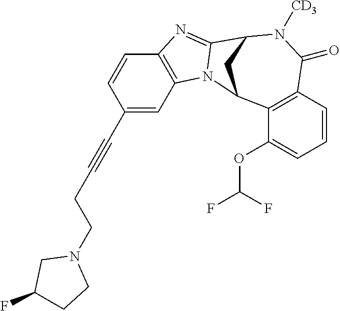 [SMILES: [2H]C([2H])([2H])N1C(=O)c2cccc(OC(F)F)c2[C@H]2C[C@@H]1c1nc3ccc(C#CCCN4CC[C@@H](F)C4)cc3n12] | (7R,14R)-1-(difluoromethoxy)-11-(4- ((R)-3-fluoropyrrolidin-1-yl)but-1-yn-1- yl)-6-(methyl-d3)-6,7-dihydro-7,14- methanobenzo[f]benzo[4,5]imidazo[1,2- a][1,4]diazocin-5(14H)-one |
| 228 | 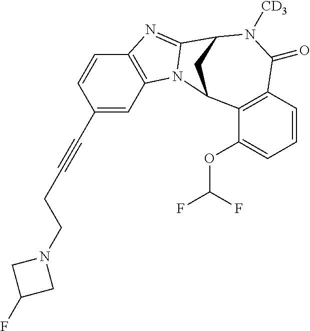 [SMILES: [2H]C([2H])([2H])N1C(=O)c2cccc(OC(F)F)c2[C@H]2C[C@@H]1c1nc3ccc(C#CCCN4CC(F)C4)cc3n12] | (7R,14R)-1-(difluoromethoxy)-11-(4-(3- fluoroazetidin-1-yl)but-1-yn-1-yl)-6- (methyl-d3)-6,7-dihydro-7,14- methanobenzo[f]benzo[4,5]imidazo[1,2- a][1,4]diazocin-5(14H)-one |
| 229 | 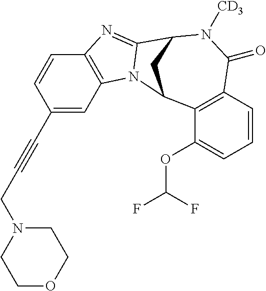 [SMILES: [2H]C([2H])([2H])N1C(=O)c2cccc(OC(F)F)c2[C@H]2C[C@@H]1c1nc3ccc(C#CCN4CCOCC4)cc3n12] | (7R,14R)-1-(difluoromethoxy)-6- (methyl-d3)-11-(3-morpholinoprop-1-yn- 1-yl)-6,7-dihydro-7,14- methanobenzo[f]benzo[4,5]imidazo[1,2- a][1,4]diazocin-5(14H)-one |
| 230 | 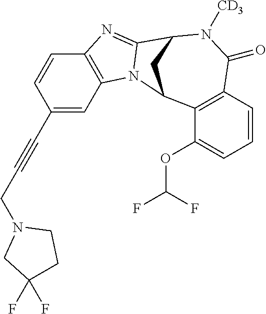 [SMILES: [2H]C([2H])([2H])N1C(=O)c2cccc(OC(F)F)c2[C@H]2C[C@@H]1c1nc3ccc(C#CCN4CCC(F)(F)C4)cc3n12] | (7R,14R)-1-(difluoromethoxy)-11-(3- (3,3-difluoropyrrolidin-1-yl)prop-1-yn-1- yl)-6-(methyl-d3)-6,7-dihydro-7,14- methanobenzo[f]benzo[4,5]imidazo[1,2- a][1,4]diazocin-5(14H)-one |
| 231 | 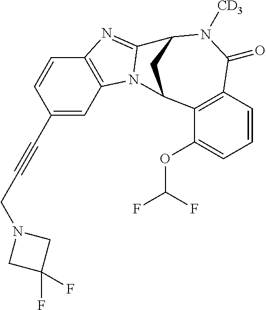 [SMILES: [2H]C([2H])([2H])N1C(=O)c2cccc(OC(F)F)c2[C@H]2C[C@@H]1c1nc3ccc(C#CCN4CC(F)(F)C4)cc3n12] | (7R,14R)-11-(3-(3,3-difluoroazetidin-1- yl)prop-1-yn-1-yl)-1-(difluoromethoxy)- 6-(methyl-d3)-6,7-dihydro-7,14- methanobenzo[f]benzo[4,5]imidazo[1,2- a][1,4]diazocin-5(14H)-one |
| 232 | 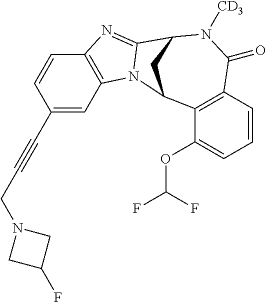 [SMILES: [2H]C([2H])([2H])N1C(=O)c2cccc(OC(F)F)c2[C@H]2C[C@@H]1c1nc3ccc(C#CCN4CC(F)C4)cc3n12] | (7R,14R)-1-(difluoromethoxy)-11-(3-(3- fluoroazetidin-1-yl)prop-1-yn-1-yl)-6- (methyl-d3)-6,7-dihydro-7,14- methanobenzo[f]benzo[4,5]imidazo[1,2- a][1,4]diazocin-5(14H)-one |
| 233 | 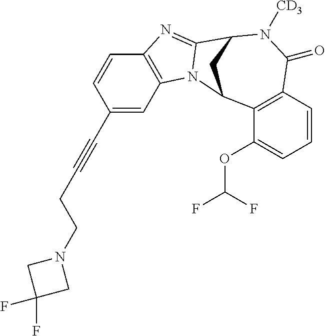 [SMILES: [2H]C([2H])([2H])N1C(=O)c2cccc(OC(F)F)c2[C@H]2C[C@@H]1c1nc3ccc(C#CCCN4CC(F)(F)C4)cc3n12] | (7R,14R)-11-(4-(3,3-difluoroazetidin-1- yl)but-1-yn-1-yl)-1-(difluoromethoxy)-6- (methyl-d3)-6,7-dihydro-7,14- methanobenzo[f]benzo[4,5]imidazo[1,2- a][1,4]diazocin-5(14H)-one |
| 234 | 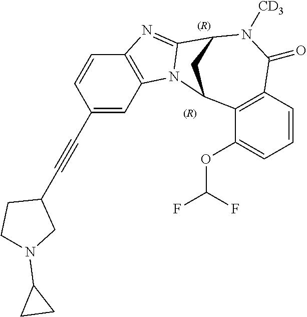 | (7R,14R)-11-((1-cyclopropylpyrrolidin- 3-yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 235 | 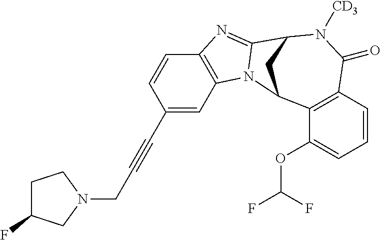 [SMILES: [2H]C([2H])([2H])N1C(=O)c2cccc(OC(F)F)c2[C@H]2C[C@@H]1c1nc3ccc(C#CCN4CC[C@H](F)C4)cc3n12] | (7R,14R)-1-(difluoromethoxy)-11-(3- ((S)-3-fluoropyrrolidin-1-yl)prop-1-yn-1- yl)-6-(methyl-d₃)-6,7-dihydro-7,14- methanobenzo[f]benzo[4,5]imidazo[1,2- a][1,4]diazocin-5(14H)-one |
| 236 | 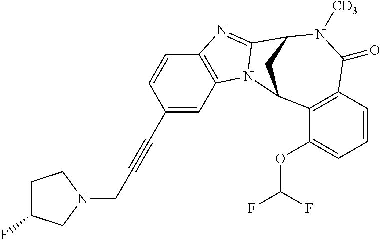 [SMILES: [2H]C([2H])([2H])N1C(=O)c2cccc(OC(F)F)c2[C@H]2C[C@@H]1c1nc3ccc(C#CCN4CC[C@@H](F)C4)cc3n12] | (7R,14R)-1-(difluoromethoxy)-11-(3- ((R)-3-fluoropyrrolidin-1-yl)prop-1-yn-1- yl)-6-(methyl-d₃)-6,7-dihydro-7,14- methanobenzo[f]benzo[4,5]imidazo[1,2- a][1,4]diazocin-5(14H)-one |
| 237 | 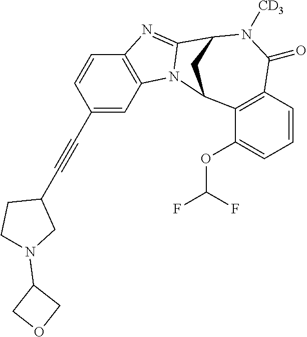 [SMILES: [2H]C([2H])([2H])N1C(=O)c2cccc(OC(F)F)c2[C@H]2C[C@@H]1c1nc3ccc(C#CC4CCN(C5COC5)C4)cc3n12] | (7R,14R)-1-(difluoromethoxy)-6- (methyl-d3)-11-((1-(oxetan-3- yl)pyrrolidin-3-yl)ethynyl)-6,7-dihydro- 7,14- methanobenzo[f]benzo[4,5]imidazo[1,2- a][1,4]diazocin-5(14H)-one |
| 238 | 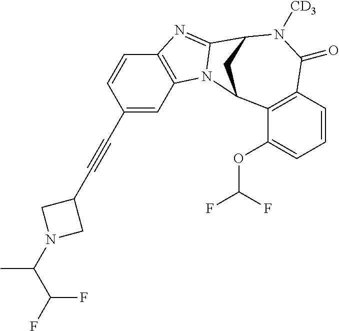 [SMILES: [2H]C([2H])([2H])N1C(=O)c2cccc(OC(F)F)c2[C@H]2C[C@@H]1c1nc3ccc(C#CC4CN(C(C)C(F)F)C4)cc3n12] | (7R,14R)-1-(difluoromethoxy)-11-((1- (1,1-difluoropropan-2-yl)azetidin-3- yl)ethynyl)-6-(methyl-d3)-6,7-dihydro- 7,14- methanobenzo[f]benzo[4,5]imidazo[1,2- a][1,4]diazocin-5(14H)-one |
| 239 and 240 | 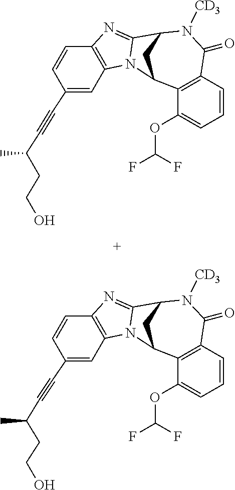 [SMILES: [2H]C([2H])([2H])N1C(=O)c2cccc(OC(F)F)c2[C@H]2C[C@@H]1c1nc3ccc(C#C[C@@H](C)CCO)cc3n12 ; [2H]C([2H])([2H])N1C(=O)c2cccc(OC(F)F)c2[C@H]2C[C@@H]1c1nc3ccc(C#C[C@H](C)CCO)cc3n12] | (7R,14R)-1-(difluoromethoxy)-11-((S or R)-5-hydroxy-3-methylpent-1-yn-1-yl)-6- (methyl-d₃)-6,7-dihydro-7,14- methanobenzo[f]benzo[4,5]imidazo[1,2- a][1,4]diazocin-5(14H)-one And (7R,14R)-1-(difluoromethoxy)-11- ((R or S)-5-hydroxy-3-methylpent-1-yn- 1-yl)-6-(methyl-d₃)-6,7-dihydro-7,14- methanobenzo[f]benzo[4,5]imidazo[1,2- a][1,4]diazocin-5(14H)-one |
| 241 | 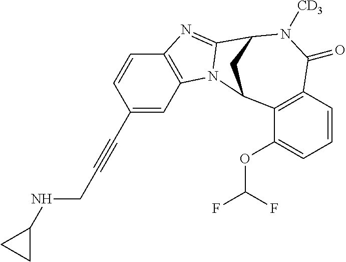 [SMILES: [2H]C([2H])([2H])N1C(=O)c2cccc(OC(F)F)c2[C@H]2C[C@@H]1c1nc3ccc(C#CCNC4CC4)cc3n12] | (7R,14R)-11-(3-(cyclopropylamino)prop- 1-yn-1-yl)-1-(difluoromethoxy)-6- (methyl-d3)-6,7-dihydro-7,14- methanobenzo[f]benzo[4,5]imidazo[1,2- a][1,4]diazocin-5(14H)-one |
| 242 | 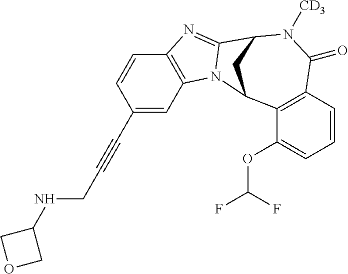 [SMILES: [2H]C([2H])([2H])N1C(=O)c2cccc(OC(F)F)c2[C@H]2C[C@@H]1c1nc3ccc(C#CCNC4COC4)cc3n12] | (7R,14R)-1-(difluoromethoxy)-6- (methyl-d3)-11-(3-(oxetan-3- ylamino)prop-1-yn-1-yl)-6,7-dihydro- 7,14- methanobenzo[f]benzo[4,5]imidazo[1,2- a][1,4]diazocin-5(14H)-one |
| 243, 244 | 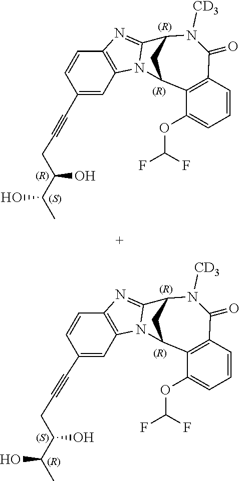 | (7R,14R)-1-(difluoromethoxy)-11-((4R or 4S,5S or 5R)-4,5-dihydroxyhex-1-yn- 1-yl)-6-(methyl-d3)-6,7-dihydro-7,14- methanobenzo[f]benzo[4,5]imidazo[1,2- a][1,4]diazocin-5(14H)-one And (7R,14R)-1-(difluoromethoxy)-11- ((4S or 4S,5R or 5S)-4,5-dihydroxyhex- 1-yn-1-yl)-6-(methyl-d3)-6,7-dihydro- 7,14- methanobenzo[f]benzo[4,5]imidazo[1,2- a][1,4]diazocin-5(14H)-one; |
| 245 and 246 | 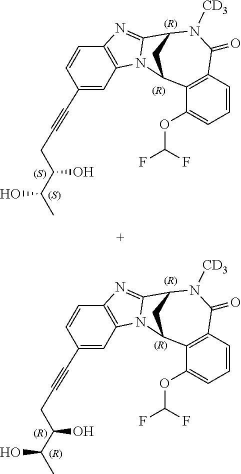 | (7R,14R)-1-(difluoromethoxy)-11-((4S or 4R,5S or 5R)-4,5-dihydroxyhex-1-yn- 1-yl)-6-(methyl-d3)-6,7-dihydro-7,14- methanobenzo[f]benzo[4,5]imidazo[1,2- a][1,4]diazocin-5(14H)-one And (7R,14R)-1-(difluoromethoxy)-11- ((4R or 4S,5R or 5S)-4,5-dihydroxyhex- 1-yn-1-yl)-6-(methyl-d3)-6,7-dihydro- 7,14- methanobenzo[f]benzo[4,5]imidazo[1,2- a][1,4]diazocin-5(14H)-one |
| 247 | 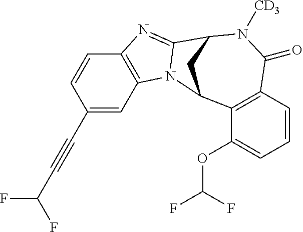 [SMILES: [2H]C([2H])([2H])N1C(=O)c2cccc(OC(F)F)c2[C@H]2C[C@@H]1c1nc3ccc(C#CC(F)F)cc3n12] | (7R,14R)-1-(difluoromethoxy)-11-(3,3- difluoroprop-1-yn-1-yl)-6-(methyl-d3)- 6,7-dihydro-7,14- methanobenzo[f]benzo[4,5]imidazo[1,2- a][1,4]diazocin-5(14H)-one |
| 248 | 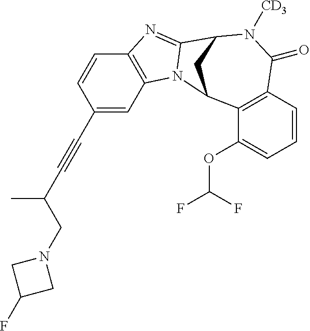 [SMILES: [2H]C([2H])([2H])N1C(=O)c2cccc(OC(F)F)c2[C@H]2C[C@@H]1c1nc3ccc(C#CC(C)CN4CC(F)C4)cc3n12] | (7R,14R)-11-(3-(cyclopropylamino)prop- 1-yn-1-yl)-1-(difluoromethoxy)-6- (methyl-d3)-6,7-dihydro-7,14- methanobenzo[f]benzo[4,5]imidazo[1,2- a][1,4]diazocin-5(14H)-one |
| 249 | 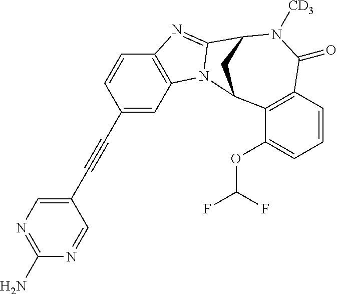 [SMILES: [2H]C([2H])([2H])N1C(=O)c2cccc(OC(F)F)c2[C@H]2C[C@@H]1c1nc3ccc(C#Cc4cnc(N)nc4)cc3n12] | (7R,14R)-11-((2-aminopyrimidin-5- yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |
| 250 | 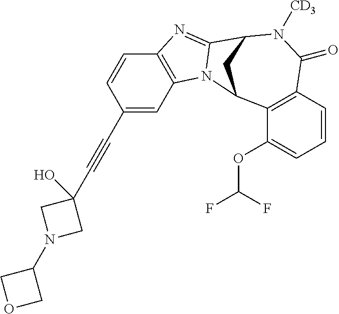 [SMILES: [2H]C([2H])([2H])N1C(=O)c2cccc(OC(F)F)c2[C@H]2C[C@@H]1c1nc3ccc(C#CC4(O)CN(C5COC5)C4)cc3n12] | (7R,14R)-1-(difluoromethoxy)-11-((3- hydroxy-1-(oxetan-3-yl)azetidin-3- yl)ethynyl)-6-(methyl-d3)-6,7-dihydro- 7,14- methanobenzo[f]benzo[4,5]imidazo[1,2- a][1,4]diazocin-5(14H)-one |
| 251 | 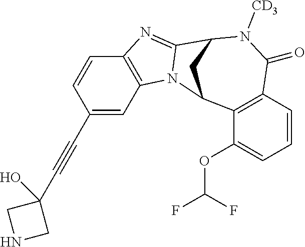 [SMILES: [2H]C([2H])([2H])N1C(=O)c2cccc(OC(F)F)c2[C@H]2C[C@@H]1c1nc3ccc(C#CC4(O)CNC4)cc3n12] | (7R,14R)-1-(difluoromethoxy)-11-((3- hydroxyazetidin-3-yl)ethynyl)-6-(methyl- d3)-6,7-dihydro-7,14- methanobenzo[f]benzo[4,5]imidazo[1,2- a][1,4]diazocin-5(14H)-one |
| 252 | 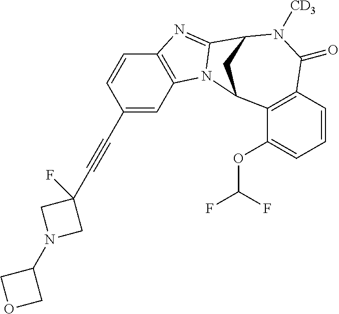 [SMILES: [2H]C([2H])([2H])N1C(=O)c2cccc(OC(F)F)c2[C@H]2C[C@@H]1c1nc3ccc(C#CC4(F)CN(C5COC5)C4)cc3n12] | (7R,14R)-1-(difluoromethoxy)-11-((3- fluoro-1-(oxetan-3-yl)azetidin-3- yl)ethynyl)-6-(methyl-d3)-6,7-dihydro- 7,14- methanobenzo[f]benzo[4,5]imidazo[1,2- a][1,4]diazocin-5(14H)-one |
| 253 | 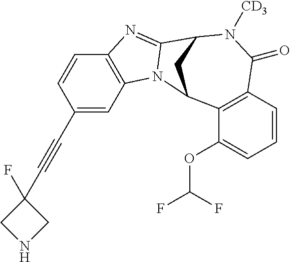 [SMILES: [2H]C([2H])([2H])N1C(=O)c2cccc(OC(F)F)c2[C@H]2C[C@@H]1c1nc3ccc(C#CC4(F)CNC4)cc3n12] | (7R,14R)-1-(difluoromethoxy)-11-((3- fluoroazetidin-3-yl)ethynyl)-6-(methyl- d3)-6,7-dihydro-7,14- methanobenzo[f]benzo[4,5]imidazo[1,2- a][1,4]diazocin-5(14H)-one |
| 254 | 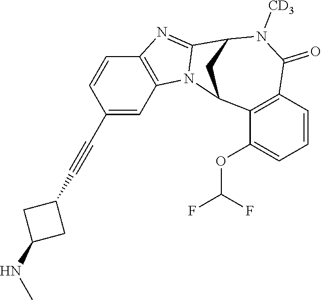 [SMILES: [2H]C([2H])([2H])N1C(=O)c2cccc(OC(F)F)c2[C@H]2C[C@@H]1c1nc3ccc(C#C[C@H]4C[C@H](NC)C4)cc3n12] | (7R,14R)-1-(difluoromethoxy)-6- (methyl-d3)-11-(((1r,3R)-3- (methylamino)cyclobutyl)ethynyl)-6,7- dihydro-7,14- methanobenzo[f]benzo[4,5]imidazo[1,2- a][1,4]diazocin-5(14H)-one |
| 255 | 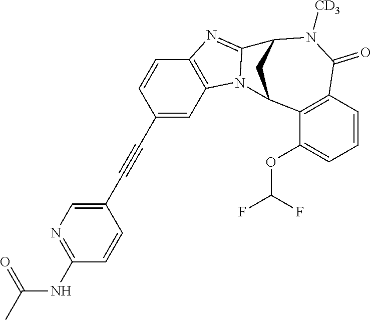 [SMILES: [2H]C([2H])([2H])N1C(=O)c2cccc(OC(F)F)c2[C@H]2C[C@@H]1c1nc3ccc(C#Cc4ccc(NC(C)=O)nc4)cc3n12] | N-(5-(((7R,14R)-1-(difluoromethoxy)-6- (methyl-d₃)-5-oxo-5,6,7,14-tetrahydro- 7,14- methanobenzo[f]benzo[4,5]imidazo[1,2- a][1,4]diazocin-11-yl)ethynyl)pyridin-2- yl)acetamide |
| 256 | 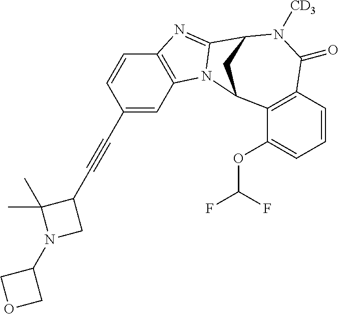 [SMILES: [2H]C([2H])([2H])N1C(=O)c2cccc(OC(F)F)c2[C@H]2C[C@@H]1c1nc3ccc(C#CC4CN(C5COC5)C4(C)C)cc3n12] | (7R,14R)-1-(difluoromethoxy)-11-((2,2- dimethyl-1-(oxetan-3-yl)azetidin-3- yl)ethynyl)-6-(methyl-d₃)-6,7-dihydro- 7,14- methanobenzo[f]benzo[4,5]imidazo[1,2- a][1,4]diazocin-5(14H)-one |
| 257 and 258 | 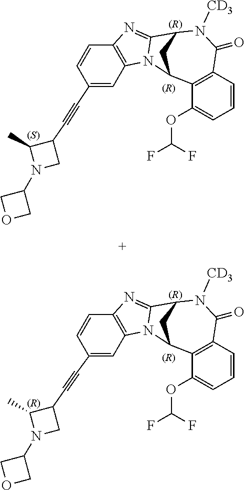 | (7R,14R)-1-(difluoromethoxy)-6- (methyl-d3)-11-(((2S or 2R)-2-methyl-1- (oxetan-3-yl)azetidin-3-yl)ethynyl)-6,7- dihydro-7,14- methanobenzo[f]benzo[4,5]imidazo[1,2- a][1,4]diazocin-5(14H)-one And (7R,14R)-1-(difluoromethoxy)-6- (methyl-d3)-11-(((2R or 2S)-2-methyl-1- (oxetan-3-yl)azetidin-3-yl)ethynyl)-6,7- dihydro-7,14- methanobenzo[f]benzo[4,5]imidazo[1,2- a][1,4]diazocin-5(14H)-one |
| 259 | 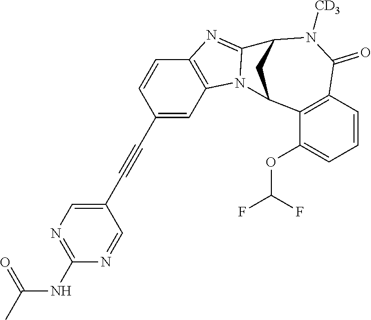 [SMILES: [2H]C([2H])([2H])N1C(=O)c2cccc(OC(F)F)c2[C@H]2C[C@@H]1c1nc3ccc(C#Cc4cnc(NC(C)=O)nc4)cc3n12] | N-(5-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)pyrimidin- 2-yl)acetamide |
| 260 | 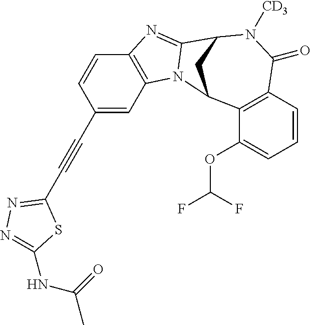 [SMILES: [2H]C([2H])([2H])N1C(=O)c2cccc(OC(F)F)c2[C@H]2C[C@@H]1c1nc3ccc(C#Cc4nnc(NC(C)=O)s4)cc3n12] | N-(5-(((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)ethynyl)-1,3,4- thiadiazol-2-yl)acetamide |
| 261 | 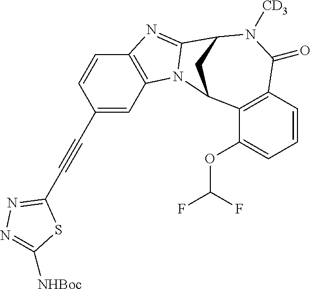 [SMILES: [2H]C([2H])([2H])N1C(=O)c2cccc(OC(F)F)c2[C@H]2C[C@@H]1c1nc3ccc(C#Cc4nnc(NC(=O)OC(C)(C)C)s4)cc3n12] | tert-butyl (5-(((7R,14R)-1- (difluoromethoxy)-6-(methyl-d3)-5-oxo- 5,6,7,14-tetrahydro-7,14- methanobenzo[f]benzo[4,5]imidazo[1,2- a][1,4]diazocin-11-yl)ethynyl)-1,3,4- thiadiazol-2-yl)carbamate |
| 262 | 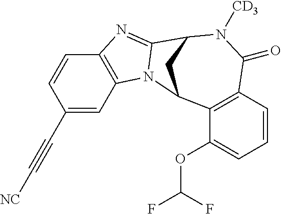 [SMILES: [2H]C([2H])([2H])N1C(=O)c2cccc(OC(F)F)c2[C@H]2C[C@@H]1c1nc3ccc(C#CC#N)cc3n12] | 3-((7R,14R)-1-(difluoromethoxy)-6- (methyl-d3)-5-oxo-5,6,7,14-tetrahydro- 7,14- methanobenzo[f]benzo[4,5]imidazo[1,2- a][1,4]diazocin-11-yl)propiolonitrile |
| 263 | 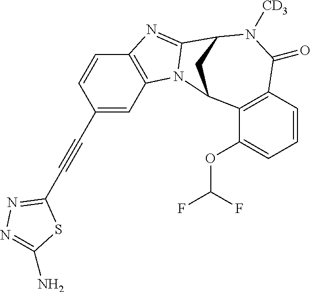 [SMILES: [2H]C([2H])([2H])N1C(=O)c2cccc(OC(F)F)c2[C@H]2C[C@@H]1c1nc3ccc(C#Cc4nnc(N)s4)cc3n12] | (7R,14R)-11-((5-amino-1,3,4-thiadiazol- 2-yl)ethynyl)-1-(difluoromethoxy)-6- (methyl-d3)-6,7-dihydro-7,14- methanobenzo[f]benzo[4,5]imidazo[1,2- a][1,4]diazocin-5(14H)-one |

Another embodiment provides a TNF-α inhibitory compound, or a pharmaceutically acceptable salt, solvate, or N-oxide thereof, having a structure presented in Table 2.

TABLE 2

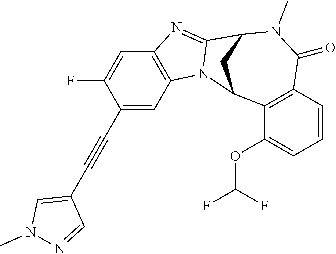 [SMILES: CN1C(=O)c2cccc(OC(F)F)c2[C@H]2C[C@@H]1c1nc3cc(F)c(C#Cc4cnn(C)c4)cc3n12]

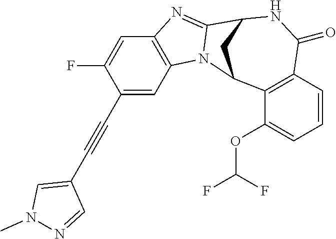 [SMILES: Cn1cc(C#Cc2cc3c(cc2F)nc2n3[C@@H]3C[C@H]2NC(=O)c2cccc(OC(F)F)c23)cn1]

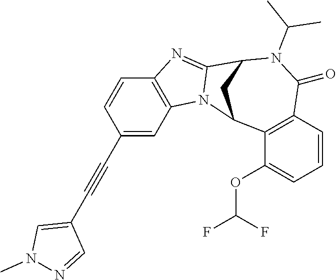 [SMILES: CC(C)N1C(=O)c2cccc(OC(F)F)c2[C@H]2C[C@@H]1c1nc3ccc(C#Cc4cnn(C)c4)cc3n12]

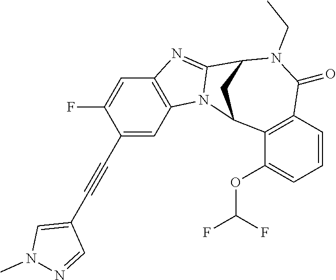 [SMILES: CCN1C(=O)c2cccc(OC(F)F)c2[C@H]2C[C@@H]1c1nc3cc(F)c(C#Cc4cnn(C)c4)cc3n12]

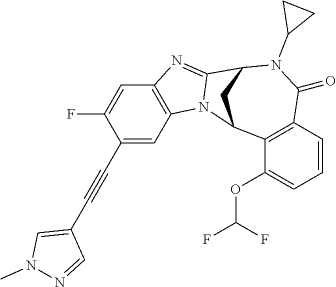 [SMILES: Cn1cc(C#Cc2cc3c(cc2F)nc2n3[C@@H]3C[C@H]2N(C2CC2)C(=O)c2cccc(OC(F)F)c23)cn1]

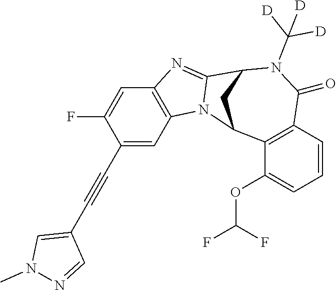 [SMILES: [2H]C([2H])([2H])N1C(=O)c2cccc(OC(F)F)c2[C@H]2C[C@@H]1c1nc3cc(F)c(C#Cc4cnn(C)c4)cc3n12]

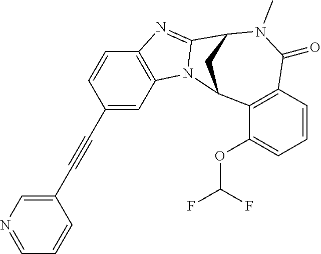 [SMILES: CN1C(=O)c2cccc(OC(F)F)c2[C@H]2C[C@@H]1c1nc3ccc(C#Cc4cccnc4)cc3n12]

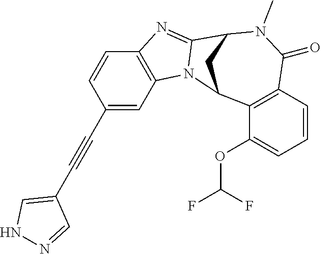 [SMILES: CN1C(=O)c2cccc(OC(F)F)c2[C@H]2C[C@@H]1c1nc3ccc(C#Cc4cn[nH]c4)cc3n12]

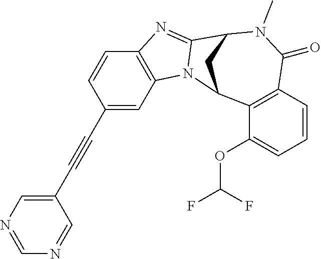 [SMILES: CN1C(=O)c2cccc(OC(F)F)c2[C@H]2C[C@@H]1c1nc3ccc(C#Cc4cncnc4)cc3n12]

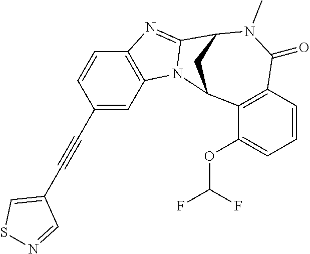 [SMILES: CN1C(=O)c2cccc(OC(F)F)c2[C@H]2C[C@@H]1c1nc3ccc(C#Cc4cnsc4)cc3n12]

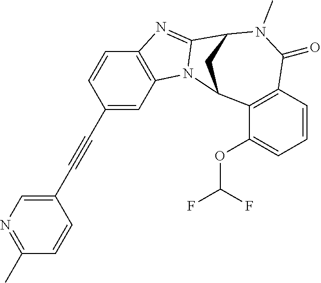 [SMILES: Cc1ccc(C#Cc2ccc3nc4n(c3c2)[C@@H]2C[C@H]4N(C)C(=O)c3cccc(OC(F)F)c32)cn1]

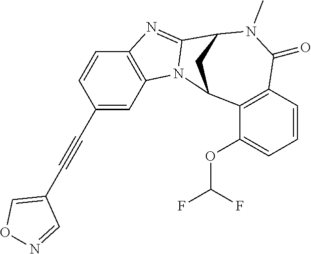 [SMILES: CN1C(=O)c2cccc(OC(F)F)c2[C@H]2C[C@@H]1c1nc3ccc(C#Cc4cnoc4)cc3n12]

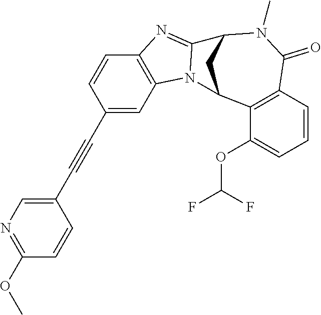 [SMILES: COc1ccc(C#Cc2ccc3nc4n(c3c2)[C@@H]2C[C@H]4N(C)C(=O)c3cccc(OC(F)F)c32)cn1]

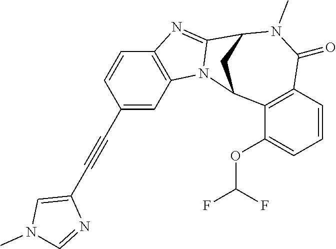 [SMILES: CN1C(=O)c2cccc(OC(F)F)c2[C@H]2C[C@@H]1c1nc3ccc(C#Cc4cn(C)cn4)cc3n12]

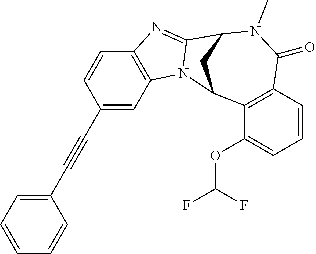 [SMILES: CN1C(=O)c2cccc(OC(F)F)c2[C@H]2C[C@@H]1c1nc3ccc(C#Cc4ccccc4)cc3n12]

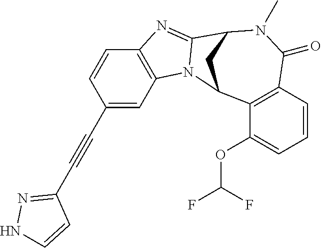 [SMILES: CN1C(=O)c2cccc(OC(F)F)c2[C@H]2C[C@@H]1c1nc3ccc(C#Cc4cc[nH]n4)cc3n12]

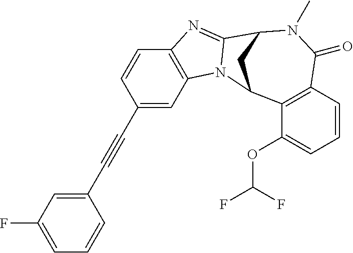 [SMILES: CN1C(=O)c2cccc(OC(F)F)c2[C@H]2C[C@@H]1c1nc3ccc(C#Cc4cccc(F)c4)cc3n12]

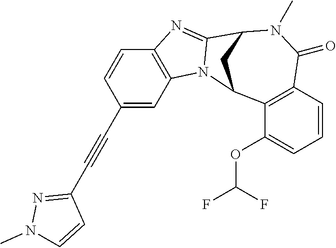 [SMILES: CN1C(=O)c2cccc(OC(F)F)c2[C@H]2C[C@@H]1c1nc3ccc(C#Cc4ccn(C)n4)cc3n12]

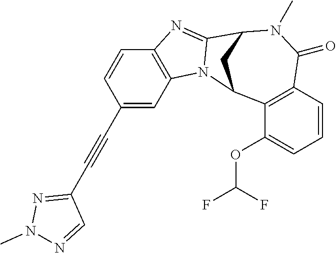 [SMILES: CN1C(=O)c2cccc(OC(F)F)c2[C@H]2C[C@@H]1c1nc3ccc(C#Cc4cnn(C)n4)cc3n12]

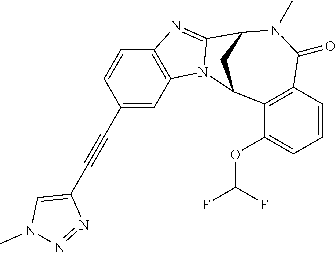 [SMILES: CN1C(=O)c2cccc(OC(F)F)c2[C@H]2C[C@@H]1c1nc3ccc(C#Cc4cn(C)nn4)cc3n12]

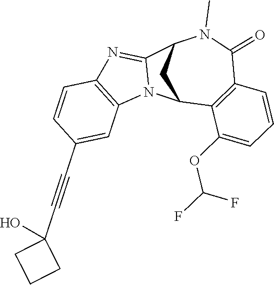 [SMILES: CN1C(=O)c2cccc(OC(F)F)c2[C@H]2C[C@@H]1c1nc3ccc(C#CC4(O)CCC4)cc3n12]

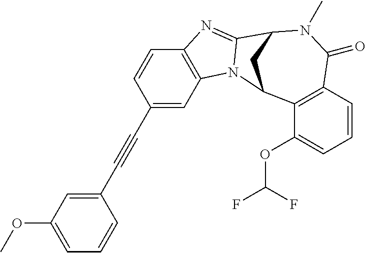 [SMILES: COc1cccc(C#Cc2ccc3nc4n(c3c2)[C@@H]2C[C@H]4N(C)C(=O)c3cccc(OC(F)F)c32)c1]

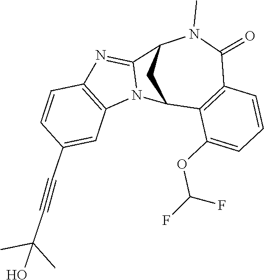 [SMILES: CN1C(=O)c2cccc(OC(F)F)c2[C@H]2C[C@@H]1c1nc3ccc(C#CC(C)(C)O)cc3n12]

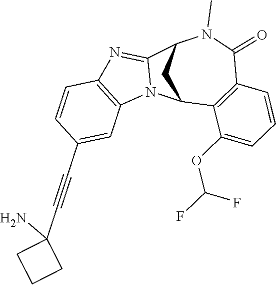 [SMILES: CN1C(=O)c2cccc(OC(F)F)c2[C@H]2C[C@@H]1c1nc3ccc(C#CC4(N)CCC4)cc3n12]

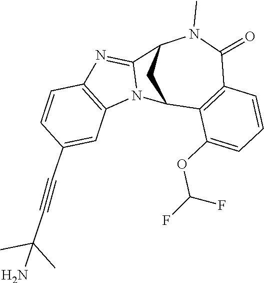 [SMILES: CN1C(=O)c2cccc(OC(F)F)c2[C@H]2C[C@@H]1c1nc3ccc(C#CC(C)(C)N)cc3n12]

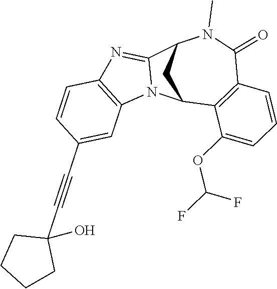 [SMILES: CN1C(=O)c2cccc(OC(F)F)c2[C@H]2C[C@@H]1c1nc3ccc(C#CC4(O)CCCC4)cc3n12]

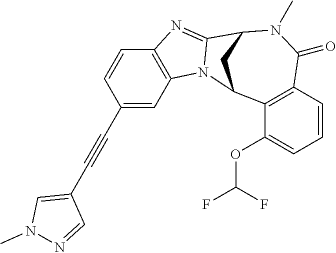 [SMILES: CN1C(=O)c2cccc(OC(F)F)c2[C@H]2C[C@@H]1c1nc3ccc(C#Cc4cnn(C)c4)cc3n12]

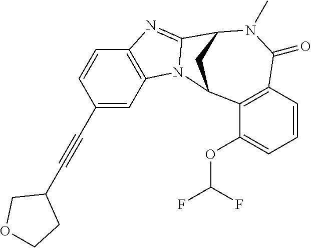 [SMILES: CN1C(=O)c2cccc(OC(F)F)c2[C@H]2C[C@@H]1c1nc3ccc(C#CC4CCOC4)cc3n12]

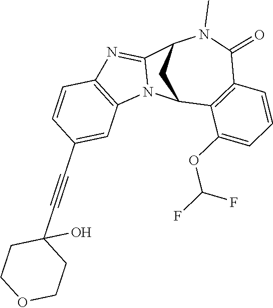 [SMILES: CN1C(=O)c2cccc(OC(F)F)c2[C@H]2C[C@@H]1c1nc3ccc(C#CC4(O)CCOCC4)cc3n12]

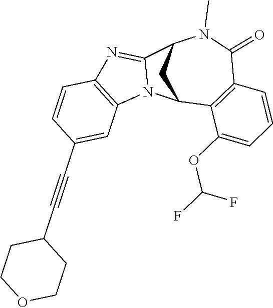 [SMILES: CN1C(=O)c2cccc(OC(F)F)c2[C@H]2C[C@@H]1c1nc3ccc(C#CC4CCOCC4)cc3n12]

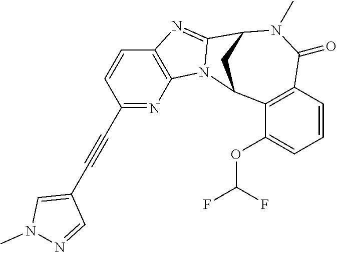 [SMILES: CN1C(=O)c2cccc(OC(F)F)c2[C@H]2C[C@@H]1c1nc3ccc(C#Cc4cnn(C)c4)nc3n12]

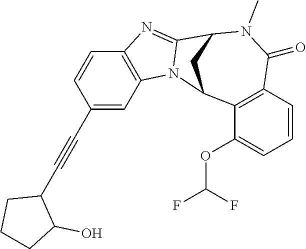 [SMILES: CN1C(=O)c2cccc(OC(F)F)c2[C@H]2C[C@@H]1c1nc3ccc(C#CC4CCCC4O)cc3n12]

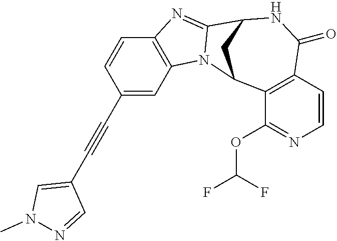

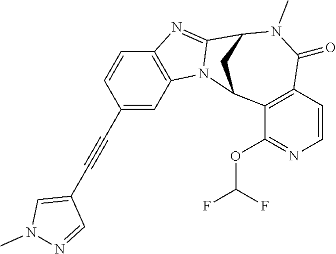 [SMILES: CN1C(=O)c2ccnc(OC(F)F)c2[C@H]2C[C@@H]1c1nc3ccc(C#Cc4cnn(C)c4)cc3n12]

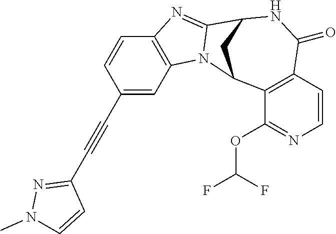 [SMILES: Cn1ccc(C#Cc2ccc3nc4n(c3c2)[C@@H]2C[C@H]4NC(=O)c3ccnc(OC(F)F)c32)n1]

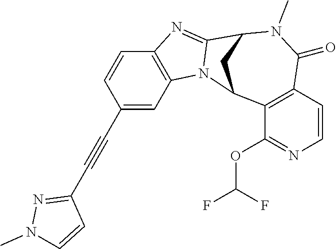 [SMILES: CN1C(=O)c2ccnc(OC(F)F)c2[C@H]2C[C@@H]1c1nc3ccc(C#Cc4ccn(C)n4)cc3n12]

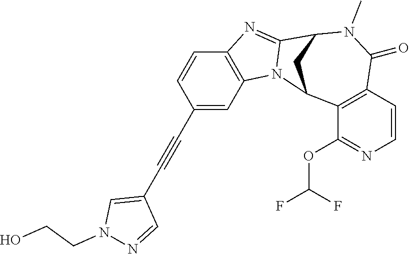 [SMILES: CN1C(=O)c2ccnc(OC(F)F)c2[C@H]2C[C@@H]1c1nc3ccc(C#Cc4cnn(CCO)c4)cc3n12]

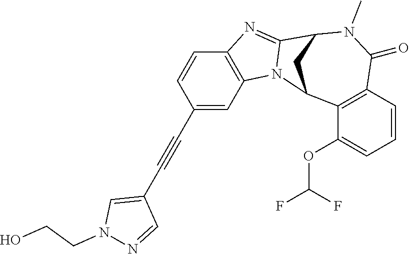 [SMILES: CN1C(=O)c2cccc(OC(F)F)c2[C@H]2C[C@@H]1c1nc3ccc(C#Cc4cnn(CCO)c4)cc3n12]

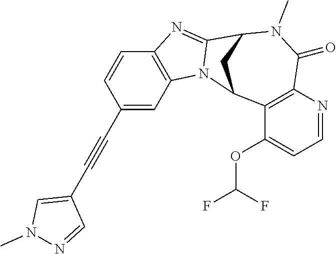 [SMILES: CN1C(=O)c2nccc(OC(F)F)c2[C@H]2C[C@@H]1c1nc3ccc(C#Cc4cnn(C)c4)cc3n12]

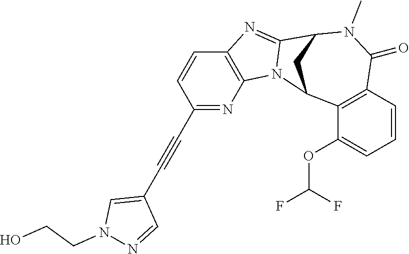 [SMILES: CN1C(=O)c2cccc(OC(F)F)c2[C@H]2C[C@@H]1c1nc3ccc(C#Cc4cnn(CCO)c4)nc3n12]

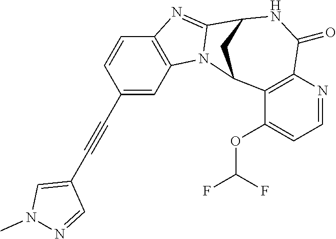 [SMILES: Cn1cc(C#Cc2ccc3nc4n(c3c2)[C@@H]2C[C@H]4NC(=O)c3nccc(OC(F)F)c32)cn1]

—

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the TNF-α inhibitory compound described herein is administered as a pure chemical. In other embodiments, the TNF-α inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one TNF-α inhibitory compound as described herein, or pharmaceutically acceptable salt, solvate, N-oxide, or deuteroisotope thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I) or (II), or pharmaceutically acceptable salt, solvate, N-oxide, or deuteroisotope thereof.

One embodiment provides a method of preparing a pharmaceutical composition comprising mixing a compound of Formula (I) or (II), or a pharmaceutically acceptable salt, solvate, or N-oxide thereof, and a pharmaceutically acceptable carrier.

In certain embodiments, the TNF-α inhibitory compound as described by Formula (I) or (II), or a pharmaceutically acceptable salt, solvate, or N-oxide thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt, solvate, or N-oxide thereof.

One embodiment provides a method of preparing a pharmaceutical composition comprising mixing a compound of Table 1 or Table 2, or a pharmaceutically acceptable salt, solvate, or N-oxide thereof, and a pharmaceutically acceptable carrier.

In certain embodiments, the TNF-α inhibitory compound as described by Table 1 or Table 2, or a pharmaceutically acceptable salt, solvate, or N-oxide thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the TNF-α inhibitory compound as described by Formula (I) or Table 1 or Table 2, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one TNF-α inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I) or (II), or a pharmaceutically acceptable salt, solvate, or N-oxide thereof, for use in a method of treatment of the human or animal body.

One embodiment provides a compound of Formula (I) or (II), or a pharmaceutically acceptable salt, solvate, or N-oxide thereof, for use in a method of treatment of inflammatory or autoimmune disease or disorder. Another embodiment provides a compound of Formula (I) or (II), or a pharmaceutically acceptable salt, solvate, or N-oxide thereof, for use in a method of treatment of inflammatory disease or disorder. Yet another embodiment provides a compound of Formula (I) or (II), or a pharmaceutically acceptable salt, solvate, or N-oxide thereof, for use in a method of treatment of autoimmune disease or disorder.

One embodiment provides a pharmaceutical composition comprising a compound of Formula (I) or (II), or a pharmaceutically acceptable salt, solvate, or N-oxide thereof, and a pharmaceutically acceptable excipient for use in a method of treatment of inflammatory or autoimmune disease or disorder.

One embodiment provides a use of a compound of Formula (I) or (II), or a pharmaceutically acceptable salt, solvate, or N-oxide thereof, in the manufacture of a medicament for the treatment of inflammatory or autoimmune disease or disorder.

In some embodiments is provided a method of treating an inflammatory or autoimmune disease or disorder, in a patient in need thereof, comprising administering to the patient a compound of Formula (I) or (II), or a pharmaceutically acceptable salt, solvate, or N-oxide thereof. In some embodiments is provided a method of treating inflammatory or autoimmune disease or disorder, in a patient in need thereof, comprising administering to the patient a pharmaceutical composition comprising a compound of Formula (I) or (II), or a pharmaceutically acceptable salt, solvate, or N-oxide thereof, and a pharmaceutically acceptable excipient. One embodiment provides a method of treating an inflammatory disease or disorder. Another embodiment provides a method of treating an autoimmune disease or disorder.

One embodiment provides a compound of Table 1 or Table 2, or a pharmaceutically acceptable salt, solvate, or N-oxide thereof, for use in a method of treatment of the human or animal body.

One embodiment provides a compound of Table 1 or Table 2, or a pharmaceutically acceptable salt, solvate, or N-oxide thereof, for use in a method of treatment of inflammatory or autoimmune disease or disorder.

One embodiment provides a pharmaceutical composition comprising a compound of Table 1 or Table 2, or a pharmaceutically acceptable salt, solvate, or N-oxide thereof, and a pharmaceutically acceptable excipient for use in a method of treatment of inflammatory or autoimmune disease or disorder.

One embodiment provides a use of a compound of Table 1 or Table 2, or a pharmaceutically acceptable salt, solvate, or N-oxide thereof, in the manufacture of a medicament for the treatment of inflammatory or autoimmune disease or disorder.

In some embodiments is provided a method of treating an inflammatory or autoimmune disease or disorder in a patient in need thereof, comprising administering to the patient a compound of Table 1 or Table 2, or a pharmaceutically acceptable salt, solvate, or N-oxide thereof. In some embodiments is provided a method of treating an inflammatory or autoimmune disease or disorder, in a patient in need thereof, comprising administering to the patient a pharmaceutical composition comprising a compound of Table 1 or Table 2, or a pharmaceutically acceptable salt, solvate, or N-oxide thereof, and a pharmaceutically acceptable excipient.

In some embodiments the inflammatory and autoimmune disease or disorder is selected from, but are not limited to: rheumatoid arthritis, psoriatic arthritis, systemic onset juvenile idiopathic arthritis, multiple sclerosis, lupus nephritis, systemic lupus erythematosus, psoriasis, Crohn's disease, colitis, asthma, graft versus host disease, allograft rejection, chronic obstructive pulmonary disease, multiple sclerosis, Alzheimer's disease, Graves' disease, cutaneous lupus, ankylosing spondylitis, cryopyrin-associated periodic syndromes (CAPS), gout, and gouty arthritis, ulcerative TNF receptor associated periodic syndrome (TRAPS), Wegener's granulomatosis, sarcoidosis, familial Mediterranean fever (FMF), neuropathic pain, and adult onset stills.

In some embodiments the inflammatory and autoimmune disease or disorder is selected from Rheumatoid Arthritis (RA), Psoriatic Arthritis (PsA), Psoriasis, Juvenile Idiopathic Arthritis (JIA), Ankylosing Spondylitis (AS) Inflammatory Bowel Diseases (IBD) including Crohn's disease and ulcerative colitis. Systemic Lupus Erythematosus (SLE), Spondyloarthritis (SpA). Behçet's Disease, Sarcoidosis, Multiple Sclerosis (MS), Palmoplantar Pustulosis (PPP), Palmoplantar Pustulosis (PPP), Atopic Dermatitis, graft versus host disease, Sepsis, Neuropathic pain, Fibromyalgia, Schnitzler Syndrome, Blau Syndrome, TNF Receptor-Associated Periodic Syndrome (TRAPS), Pyogenic Arthritis, Pyoderma Gangrenosum, chronic Recurrent Multifocal Osteomyelitis (CRMO).

Provided herein is the method wherein the pharmaceutical composition is administered orally. Provided herein is the method wherein the pharmaceutical composition is administered by injection.

One embodiment provides a method of inhibiting TNF-α activity comprising contacting the TNF-α protein with a compound of Formula (I) or (II), or Table 1 or Table 2. Another embodiment provides the method of inhibiting TNF-α activity, wherein the TNF-α protein is contacted in an in vivo setting. Another embodiment provides the method of inhibiting TNF-α activity, wherein the TNF-α protein is contacted in an in vitro setting.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the TNF-α inhibitory compounds disclosed herein are synthesized according to the following examples. As used below, and throughout the description of the invention, the following abbreviations, unless otherwise indicated, shall be understood to have the following meanings:
ACN acetonitrile
° C. degrees Celsius
δ_{H} chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP Triphenylphosphine

### Example 1: (1R,11R)-18-(difluoromethoxy)-12-methyl-5-[2-(1-methylpyrazol-4-yl)ethynyl]-2,9,12-triazapentacyclo[9.8.1.0\hat{ }{2,10}.0\hat{ }{3,8}.0\hat{ }{14,19})]icosa-3(8),4,6,9,14(19),15,17-heptaen-13-one

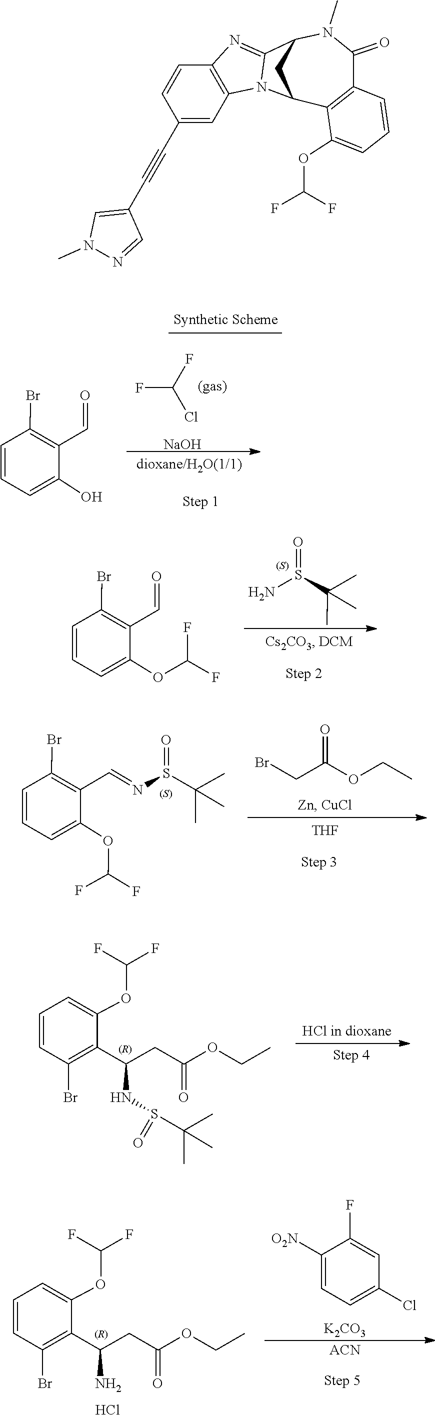

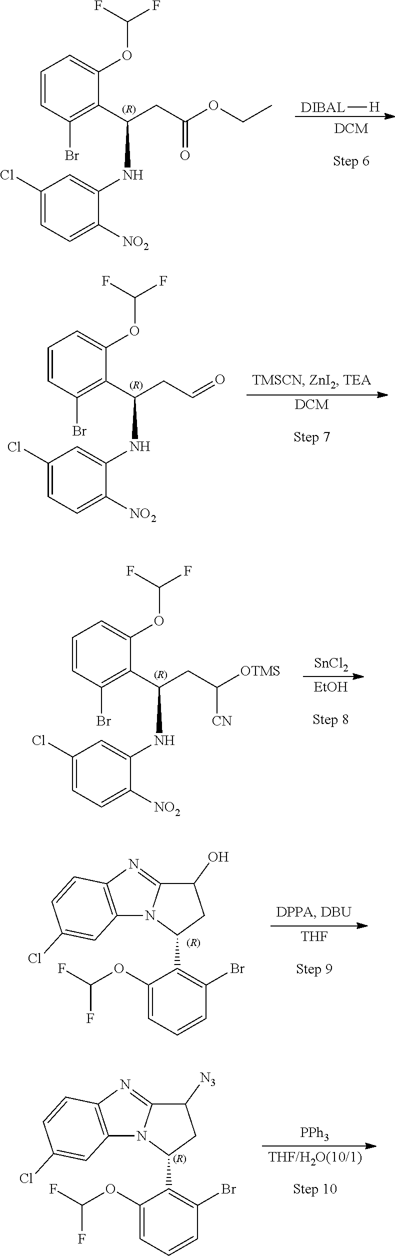

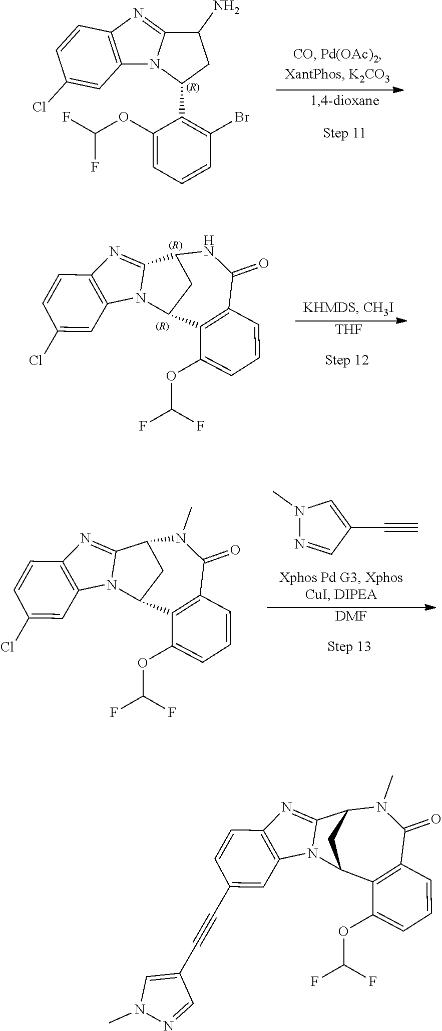

### Preparation 1A: 2-bromo-6-(difluoromethoxy)benzaldehyde

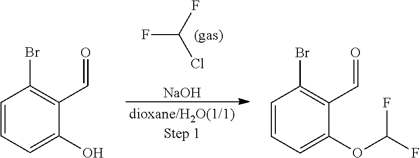

To a stirred solution of 2-bromo-6-hydroxybenzaldehyde (90.00 g, 447.719 mmol) in 1,4-dioxane (900 mL) was added the solution of NaOH (107.44 g, 2686.314 mmol) in H₂O (900 mL) dropwise at room temperature. The mixture was heated at 65° C. and chlorodifluoromethane (gas) was passed through the solution. The mixture was stirred for 1 h at 65° C. The reaction mixture was allowed to cool down to room temperature. The resulting mixture was filtered, and the filter cake was washed with EtOAc (3×500 mL). The filtrate was extracted with EtOAc (3×500 mL) and combined organic layers were dried over anhydrous Na₂SO₄. After filtration, the filtrate was concentrated under reduced pressure. The residue was purified by silica gel column chromatography, eluted with PE/EA (20:1) to afford 2-bromo-6-(difluoromethoxy)benzaldehyde (80.00 g, 71%) as a yellow oil. ¹H NMR (300 MHz, Chloroform-d) δ 10.35 (s, 1H), 7.58 (d, J=8.1 Hz, 1H), 7.41 (t, J=8.1 Hz, 1H), 7.26 (d, J=7.9 Hz, 1H), 6.61 (t, J=73.4 Hz, 1H).

### Preparation 1B: (S)—N-{[2-bromo-6-(difluoromethoxy)phenyl]methylidene}-2-methylpropane-2-sulfinamide

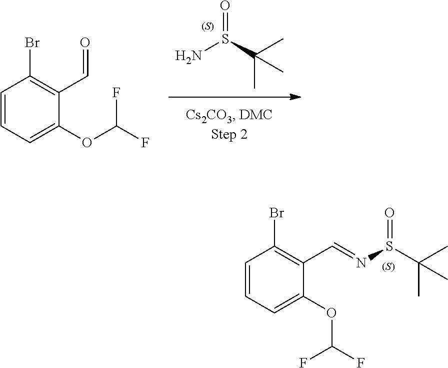

To a stirred solution of 2-bromo-6-(difluoromethoxy)benzaldehyde (80.00 g, 318.691 mmol) and (S)-2-methylpropane-2-sulfinamide (38.63 g, 318.691 mmol) in CH₂Cl₂ (800 mL) was added Cs₂CO₃ (207.67 g, 637.382 mmol) at room temperature. The mixture was stirred for 16 h at room temperature. The resulting mixture was diluted with water (1 L) and extracted with EtOAc (3×500 mL). The combined organic layers were washed with brine (1×1 L), dried over anhydrous Na₂SO₄. After filtration, the filtrate was concentrated under reduced pressure. The residue was purified by silica gel column chromatography, eluted with PE/EA (10:1) to afford (S)—N-{[2-bromo-6-(difluoromethoxy)phenyl]methylidene}-2-methylpropane-2-sulfinamide (90.00 g, 80%) as a yellow oil. MS ESI calculated for C₁₂H₁₄BrF₂NO₂S [M+H]⁺ 353.99 355.99. found 353.80 355.75. ¹H NMR (300 MHz, Chloroform-d) δ 8.84 (s, 1H), 7.59-7.57 (m, 1H), 7.35-7.31 (m, 1H), 7.26-7.24 (m, 1H), 6.57 (t, J=73.8 Hz, 1H), 1.30 (s, 9H).

### Preparation 1C: ethyl (3R)-3-[2-bromo-6-(difluoromethoxy)phenyl]-3-{[(S)-2-methylpropane-2-sulfinyl]amino}propanoate

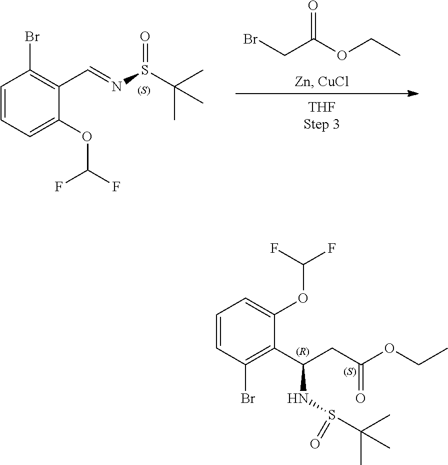

To a stirred mixture of activated Zn powder (36.92 g, 564.640 mmol) in THF (200 mL) was added anhydrous CuCl (5.59 g, 56.464 mmol) at room temperature. The mixture was stirred for 0.5 h at 70° C. The mixture was allowed to cool down to room temperature. To the above mixture was added solution of ethyl bromoacetate (23.57 g, 141.160 mmol) in THF (200 mL) dropwise at room temperature. The resulting mixture was stirred for additional 0.5 h at 50° C. The resulting mixture was filtered. To the above filtrate was added solution of (S)—N-{[2-bromo-6-(difluoromethoxy)phenyl]methylidene}-2-methylpropane-2-sulfinamide (20.00 g, 56.464 mmol) in THF (20 mL) dropwise at 0° C. The resulting mixture was stirred for additional 2 h at room temperature. This reaction was quenched with sat. NH₄Cl (aq.) at room temperature. The resulting mixture was filtered, and the filter cake was washed with ethyl acetate (3×200 mL). The filtrate was extracted with EtOAc (3×200 mL). The combined organic layers were washed with brine (1×500 mL), dried over anhydrous Na₂SO₄. After filtration, the filtrate was concentrated under reduced pressure. The residue was purified by silica gel column chromatography, eluted with CH₂Cl₂/MeOH (10:1) to afford ethyl (3R)-3-[2-bromo-6-(difluoromethoxy)phenyl]-3-{[(S)-2-methylpropane-2-sulfinyl]amino}propanoate (21.00 g, 84%) as a yellow oil. MS ESI calculated for C₁₆H₂₂BrF₂NO₄S [M+H]⁺ 442.04 444.4. found 441.90 443.90. ¹H NMR (300 MHz, Chloroform-d) δ 7.50-7.42 (m, 1H), 7.23-7.00 (m, 2H), 6.62 (t, J=73.0 Hz, 1H), 5.68-5.55 (m, 1H), 4.18-4.03 (m, 2H), 3.36-2.92 (m, 2H), 1.22 (t, J=7.0 Hz, 3H), 1.16 (s, 9H).

### Preparation 1D: ethyl (3R)-3-amino-3-[2-bromo-6-(difluoromethoxy)phenyl]propanoate Hydrochloride

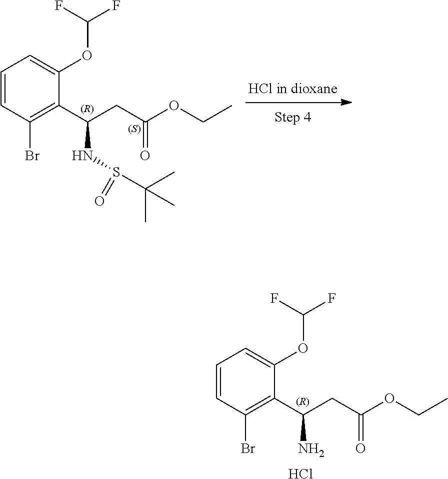

To a stirred solution of ethyl (3R)-3-[2-bromo-6-(difluoromethoxy)phenyl]-3-{[(S)-2-methylpropane-2-sulfinyl]amino}propanoate (24.00 g, 54.259 mmol) in Et₂O (50 mL) and EtOH (25 mL) was added 4 N HCl(gas) in 1,4-dioxane (75 mL) at room temperature. The mixture was stirred for 3 h at room temperature. The resulting mixture was concentrated under reduced pressure. This resulting in ethyl (3R)-3-amino-3-[2-bromo-6-(difluoromethoxy)phenyl]propanoate hydrochloride (20.00 g, 98%) as a yellow oil. MS ESI calculated for C₁₂H₁₄BrF₂NO₃ [M+H]⁺ 338.01 340.01. found 338.00 340.00.

### Preparation 1E: ethyl (3R)-3-[2-bromo-6-(difluoromethoxy)phenyl]-3-[(5-chloro-2-nitrophenyl)amino]propanoate

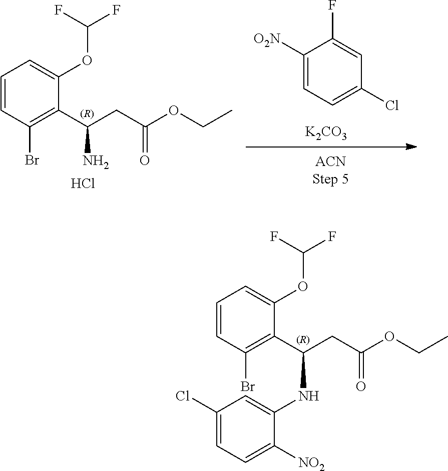

To a stirred solution of ethyl (3R)-3-amino-3-[2-bromo-6-(difluoromethoxy)phenyl]propanoate hydrochloride (20.00 g, 53.389 mmol) and 4-chloro-2-fluoro-1-nitrobenzene (11.25 g, 64.067 mmol) in ACN (200 mL) was added potassium carbonate (22.30 g, 160.167 mmol) at room temperature. The mixture was stirred for 16 h at 80° C. The resulting mixture was diluted with water (200 mL), and extracted with EtOAc (3×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EA (10:1) to afford ethyl (3R)-3-[2-bromo-6-(difluoromethoxy)phenyl]-3-[(5-chloro-2-nitrophenyl)amino]propanoate (20.00 g, 76%) as a yellow oil. MS ESI calculated for C₁₈H₁₆BrClF₂N₂O₅ [M+H]⁺ 492.99 494.99. found 492.95 494.85. ¹H NMR (400 MHz, Chloroform-d) δ 8.92 (d, J=8.8 Hz, 1H), 8.08 (d, J=9.1 Hz, 1H), 7.48-7.43 (m, 1H), 7.23-7.11 (m, 2H), 7.11-7.04 (m, 1H), 6.65 (t, J=73.0 Hz, 1H), 6.63-6.57 (m, 1H), 5.87-5.77 (m, 1H), 4.18-4.09 (m, 2H), 3.23-3.17 (m, 1H), 3.02-2.85 (m, 1H), 1.22 (t, J=7.1 Hz, 3H).

### Preparation 1F: (3R)-3-[2-bromo-6-(difluoromethoxy)phenyl]-3-[(5-chloro-2-nitrophenyl)amino]propanal

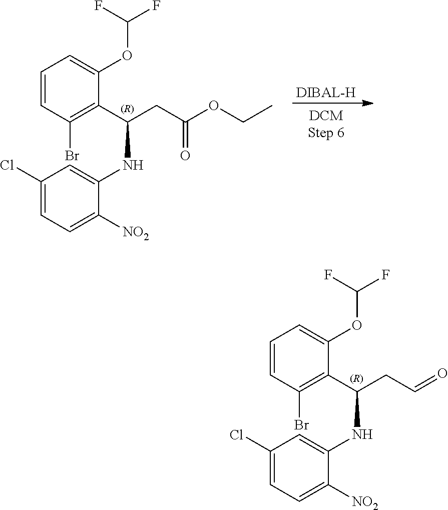

To a stirred solution of ethyl (3R)-3-[2-bromo-6-(difluoromethoxy)phenyl]-3-[(5-chloro-2-nitrophenyl)amino]propanoate (18.00 g, 36.460 mmol) in CH₂Cl₂ (200 mL) was added 1N DIBAL-H (73 mL, 73.000 mmol) in THF dropwise at −78° C. under nitrogen atmosphere. The reaction was stirred for 3 h at −78° C. under nitrogen atmosphere. The resulting mixture was quenched with sat. NH₄Cl (aq.) at −78° C., and extracted with CH₂Cl₂ (3×500 mL). The combined organic layers were dried over anhydrous Na₂SO₄. After filtration, the filtrate was concentrated under reduced pressure. The residue was purified by silica gel column chromatography, eluted with PE/EA (10:1) to afford (3R)-3-[2-bromo-6-(difluoromethoxy)phenyl]-3-[(5-chloro-2-nitrophenyl)amino]propanal (12.00 g, 73%) as a yellow oil. MS ESI calculated for C₁₆H₁₂BrClF₂N₂O₄ [M+H]⁺ 448.96 450.96. found 448.95 450.95. ¹H NMR (400 MHz, Chloroform-d) δ 9.81 (s, 1H), 8.84 (d, J=9.0 Hz, 1H), 8.08 (d, J=9.1 Hz, 1H), 7.49-7.43 (m, 1H), 7.22-7.14 (m, 2H), 7.13-7.06 (m, 1H), 6.67 (t, J=73.0 Hz, 1H), 6.65-6.60 (m, 1H), 5.97-5.88 (m, 1H), 3.56-3.41 (m, 1H), 3.22-2.93 (m, 1H).

### Preparation 1G: (4R)-4-[2-bromo-6-(difluoromethoxy)phenyl]-4-[(5-chloro-2-nitrophenyl)amino]-2-[(trimethylsilyl)oxy]butanenitrile

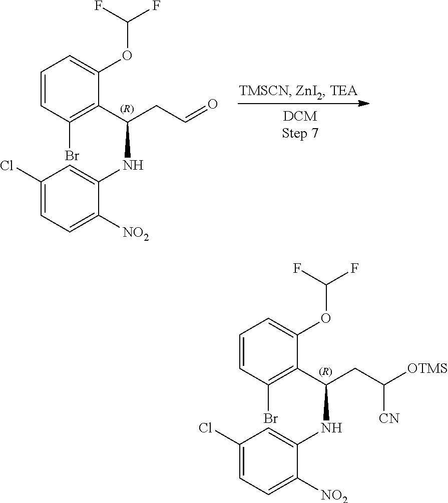

To a stirred solution of (3R)-3-[2-bromo-6-(difluoromethoxy)phenyl]-3-[(5-chloro-2-nitrophenyl)amino]propanal (12.00 g, 26.689 mmol) in CH₂Cl₂ (120 mL) were added ZnI₂ (852 mg, 2.669 mmol), TMSCN (5.30 g, 53.378 mmol) and TEA (0.37 mL, 2.669 mmol) at room temperature. The mixture was stirred for 16 h at room temperature. The resulting mixture was diluted with water (50 mL), and extracted with CH₂Cl₂ (3×50 mL). The combined organic layers were dried over anhydrous Na₂SO₄. After filtration, the filtrate was concentrated under reduced pressure to afford (4R)-4-[2-bromo-6-(difluoromethoxy)phenyl]-4-[(5-chloro-2-nitrophenyl)amino]-2-[(trimethylsilyl)oxy]butanenitrile (13.00 g, 89%) as a yellow oil. The crude product was used in the next step directly without further purification. MS ESI calculated for C₂₀H₂₁BrClF₂N₃O₄Si [M+H]⁺ 548.01 550.01. found 548.05 550.00.

### Preparation 1H: (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol

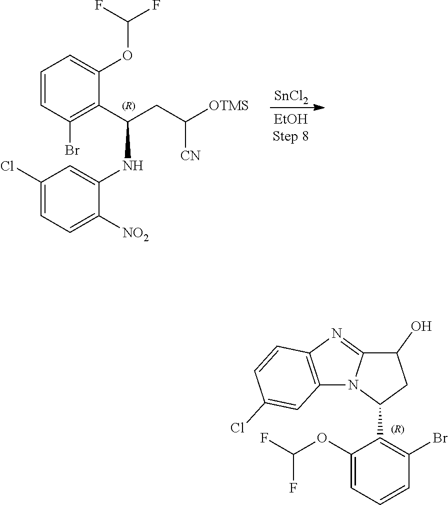

To a stirred solution of (4R)-4-[2-bromo-6-(difluoromethoxy)phenyl]-4-[(5-chloro-2-nitrophenyl)amino]-2-[(trimethylsilyl)oxy]butanenitrile (13.00 g, 23.686 mmol) in EtOH (100 mL) was added SnCl₂·2H₂O (26.96 g, 118.430 mmol) at room temperature. The mixture was stirred for 16 h at 80° C. The reaction was quenched by the addition of water (50 mL) at room temperature, and basified to pH~8 with 1 N KOH (aq.). The mixture was filtered, the filter cake was washed with EtOAc (3×50 mL), and extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:2) to afford (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol (7.00 g, 69%) as a yellow solid. MS ESI calculated for C₁₇H₁₂BrClF₂N₂O₂ [M+H]⁺ 428.97 430.97. found 429.00 431.00.

### Preparation 1I: (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene

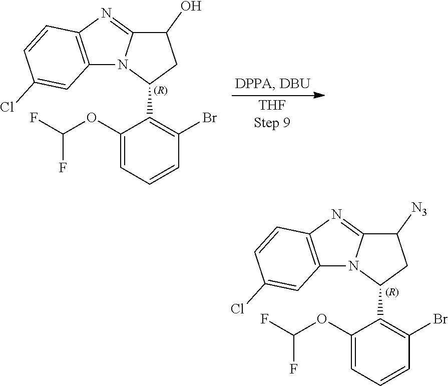

To a stirred solution of (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-ol (22.00 g, 51.204 mmol) in THF (200 mL) were added DPPA (16.91 g, 61.445 mmol) and DBU (15.59 g, 102.408 mmol) at 0° C. The mixture was stirred for 16 h at room temperature. The reaction was quenched by the addition of water (200 mL) at room temperature, and extracted with EtOAc (3×500 mL). The combined organic layers were washed with brine (3×200 mL), dried over anhydrous Na₂SO₄. After filtration, the filtrate was concentrated under reduced pressure. The residue was purified by silica gel column chromatography, eluted with CH₂Cl₂/MeOH (20:1) to afford desired mixture of cis (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene (15.00 g, 64%) as a green oil. MS ESI calculated for C₁₇H₁₁BrClF₂N₅O [M+H]⁺ 453.98 455.98. found 454.05 456.05.

### Preparation 1J: (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine

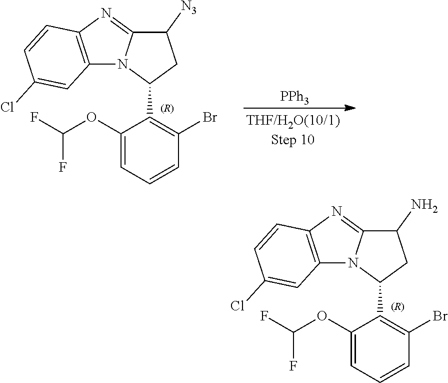

To a stirred solution of (3R)-5-azido-3-[2-bromo-6-(difluoromethoxy)phenyl]-11-chloro-2,7-diazatricyclo[6.4.0.0\hat{ }{2,6}]dodeca-1(8),6,9,11-tetraene (15.00 g, 32.992 mmol) in THF (50 mL) and H₂O (5 mL) was added PPh₃ (12.98 g, 49.488 mmol) at room temperature. The mixture was stirred for 16 h at room temperature. The resulting mixture was concentrated under reduced pressure. The residue was purified by silica gel column chromatography, eluted with CH₂Cl₂/MeOH (12:1) to afford (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine (10.00 g, 71%) as a yellow solid. MS ESI calculated for C₁₇H₁₃BrClF₂N₃O [M+H]+ 427.99 429.99. found 428.00 430.00. ¹H NMR (400 MHz, Chloroform-d) δ 7.70-7.58 (m, 2H), 7.35-7.28 (m, 1H), 7.23-7.00 (m, 2H), 6.90-6.46 (m, 1H), 6.17-6.06 (m, 1H), 5.98-5.58 (m, 1H), 4.77-4.57 (m, 1H), 3.60-3.40 (m 1H), 2.82-2.55 (m, 1H).

### Preparation 1K: (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

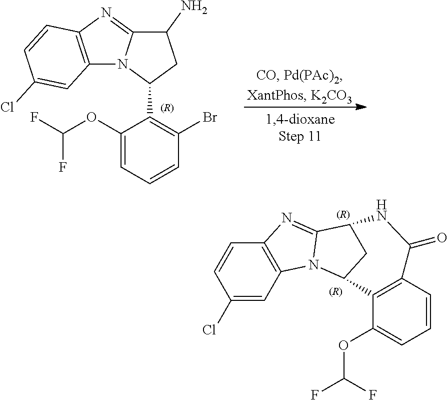

To a solution of (3R)-3-[2-bromo-6-(difluoromethoxy)phenyl]-11-chloro-2,7-diazatricyclo[6.4.0.0\hat{ }{2,6}]dodeca-1(8),6,9,11-tetraen-5-amine (500 mg, 1.166 mmol) in 1,4-dioxane (10 mL) were added K₂CO₃ (806 mg, 5.830 mmol), XantPhos (34 mg, 0.058 mmol) and Pd(OAc)₂ (13 mg, 0.058 mmol) in a pressure tank. The mixture was purged with nitrogen for 5 min and then was pressurized to 1 atm with carbon monoxide at 100° C. for 16 h. The resulting mixture was concentrated under reduced pressure. The residue was purified by silica gel column chromatography, eluted with CH₂Cl₂/MeOH (10:1) to afford a mixture of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one and (1S,11S)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (200 mg). The mixture was purified by Chiral-Prep-HPLC with the following conditions (Column: CHIRAL ART Cellulose-SB, 2*25 cm, 5 μm; Mobile Phase A: Hex (10 mM NH₃-MeOH), Mobile Phase B: EtOH; Flow rate: 20 mL/min; Gradient: 30% B; 210/286 nm). The first peak afforded 160 mg of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one as a white solid. MS ESI calculated for C₁₈H₁₂ClF₂N₃O₂ [M+H]⁺, 376.06. found 375.95. ¹H NMR (400 MHz, Chloroform-d) δ 8.44 (d, J=8.0 Hz, 1H), 7.64 (d, J=8.7 Hz, 1H), 7.52-7.35 (m, 4H), 7.24-7.19 (m, 1H), 6.85 (t, J=72.6 Hz, 1H), 6.29 (d, J=7.3 Hz, 1H), 4.94 (t, J=6.6 Hz, 1H), 3.53-3.41 (m, 1H), 2.85 (d, J=13.3 Hz, 1H).

### Preparation 1L: (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

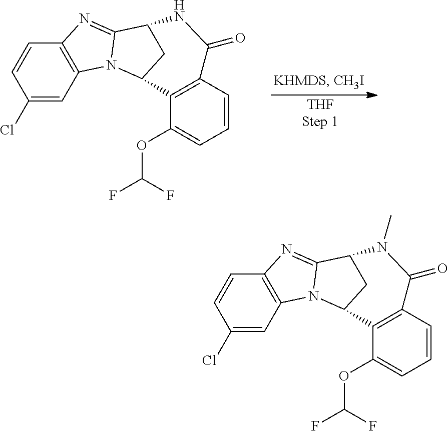 [SMILES: O=C1N[C@@H]2C[C@H](c3c(OC(F)F)cccc31)n1c2nc2ccc(Cl)cc21 ; CN1C(=O)c2cccc(OC(F)F)c2[C@H]2C[C@@H]1c1nc3ccc(Cl)cc3n12]

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (120 mg, 0.319 mmol) in dry THF (2 mL) was added 1N KHNDS (0.4 mL, 0.40 mmol) in THF dropwise at −78° C. under nitrogen atmosphere. This reaction was stirred for 1 h at −78° C. under nitrogen atmosphere. To the above solution was added CH₃I (68 mg, 0.479 mmol) dropwise over 2 min at −78° C. The mixture was stirred for 1 h at room temperature. The reaction was quenched by the addition of sat. NH₄Cl (aq.) (1 mL) at room temperature. The resulting mixture was concentrated under reduced pressure. The residue was purified by silica gel column chromatography, eluted with CH₂Cl₂/MeOH (10:1) to afford (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (85 mg, 68%) as a yellow solid. MS ESI calculated for C₁₉H₁₄ClF₂N₃O₂ [M+H]⁺, 390.07 392.07. found 390.25 392.25. ¹H NMR (300 MHz, Chloroform-d) δ 8.50 (d, J=8.2 Hz, 1H), 7.65 (d, J=8.7 Hz, 1H), 7.48-7.42 (m, 2H), 7.38-7.32 (m, 1H), 7.25-7.22 (m, 1H), 6.84 (t, J=72.6 Hz, 1H), 6.24-6.21 (m, 1H), 5.02-5.00 (m, 1H), 3.53 (s, 3H), 3.49-3.41 (m, 1H), 2.90-2.86 (m, 1H).

### Example 1: (1R,11R)-18-(difluoromethoxy)-12-methyl-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

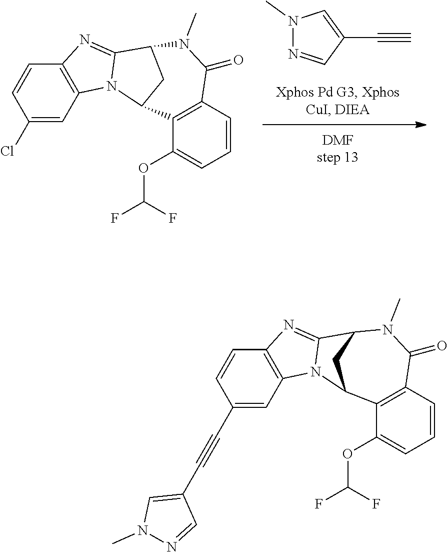

To a stirred solution of (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (80 mg, 0.205 mmol), CuI (2 mg, 0.010 mmol) and 4-ethynyl-1-methylpyrazole (109 mg, 1.025 mmol) in DMF (2 mL) were added XPhos (10 mg, 0.021 mmol), DIPEA (178 uL, 1.025 mmol) and XPhos Pd G3 (17 mg, 0.021 mmol) at room temperature under nitrogen atmosphere. The resulting mixture was stirred for overnight at 110° C. under nitrogen atmosphere. The mixture was allowed to cool down to room temperature and diluted with water (20 mL). The mixture was extracted with EtOAc (2×20 mL). The combined organic layers were washed with brine (30 mL), dried over anhydrous Na₂SO₄. After filtration, the filtrate was concentrated under reduced pressure. The residue was purified by silica gel column chromatography, eluted with CH₂Cl₂/MeOH (10/1) to afford the crude product. The crude product (75 mg) was purified by reversed-phase flash chromatography with the following conditions: column, C18 silica gel; mobile phase, CH₃CN in water (0.1% FA), 30% to 50% gradient in 20 min; detector, 254 nm to afford (1R,11R)-18-(difluoromethoxy)-12-methyl-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (10 mg, 10%) as a light yellow solid. MS ESI calculated for C₂₅H₁₉F₂N₅O₂ [M+H]⁺, 460.15. found 460.15. ¹H NMR (400 MHz, Chloroform-d) δ 8.49 (d, J=8.2 Hz, 1H), 7.69-7.62 (m, 2H), 7.58-7.54 (m, 2H), 7.42 (t, J=8.2 Hz, 1H), 7.38-7.31 (m, 2H), 6.84 (t, J=73.3 Hz, 1H), 6.22 (d, J=7.2 Hz, 1H), 4.95 (d, J=7.0 Hz, 1H), 3.92 (s, 3H), 3.51 (s, 3H), 3.50-3.40 (m, 1H), 2.86 (d, J=13.5 Hz, 1H). ¹⁹F NMR (377 MHz, Chloroform-d) δ −79.80, −80.24, −81.10, −81.54.

### Example 2: (1R,11R)-18-(difluoromethoxy)-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

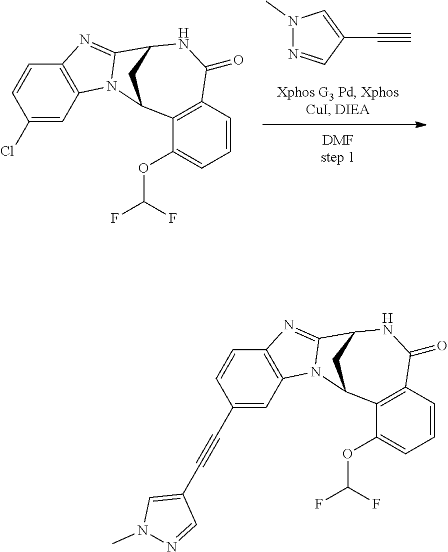

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.399 mmol) and 4-ethynyl-1-methylpyrazole (212 mg, 1.995 mmol) in DMF (3 mL) were added XPhos (19 mg, 0.040 mmol), DIPEA (257 mg, 1.995 mmol), CuI (4 mg, 0.020 mmol) and XPhos Pd G₃ (34 mg, 0.040 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm to afford (1R,11R)-18-(difluoromethoxy)-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (28 mg, 15%) as a yellow solid. MS ESI calculated for C₂₄H₁₇F₂N₅O₂ [M+H]⁺, 446.14. found 446.15. ¹H NMR (400 MHz, Chloroform-d) δ 8.47-8.40 (m, 1H), 7.71-7.59 (m, 2H), 7.59-7.52 (m, 2H), 7.49-7.33 (m, 4H), 6.85 (d, J=72.0 Hz, 1H), 6.32 (d, J=7.2 Hz, 1H), 4.97 (t, J=6.8 Hz, 1H), 3.92 (s, 3H), 3.53-3.41 (m, 1H), 2.86 (d, J=13.2 Hz, 1H). ¹⁹F NMR (377 MHz, Chloroform-d) δ −79.98, −80.43, −81.11, −81.56.

### Example 3: (1R,11R)-18-(difluoromethoxy)-5-[2-(pyrimidin-5-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

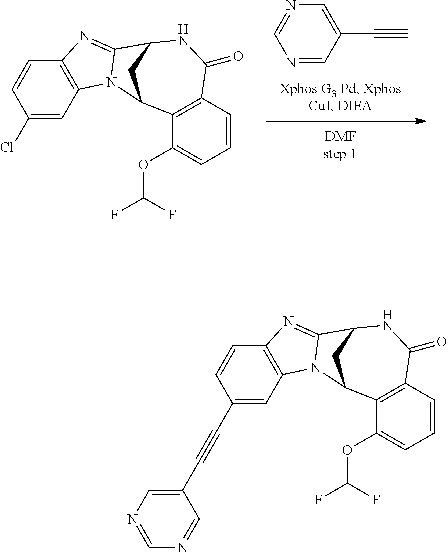

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.399 mmol) and 5-ethynylpyrimidine (208 mg, 1.995 mmol) in DMF (3 mL) were added XPhos (19 mg, 0.040 mmol), DIPEA (258 mg, 1.995 mmol), CuI (4 mg, 0.020 mmol) and XPhos Pd G3 (34 mg, 0.040 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm followed by reversed-phase flash chromatography with the following conditions: column, C18 silica gel; mobile phase, CH₃CN in water (0.1% NH₃·H₂O), 20% to 50% gradient in 25 min; detector, 254 nm to afford (1R,11R)-18-(difluoromethoxy)-5-[2-(pyrimidin-5-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (25 mg, 13%) as a white solid. MS ESI calculated for C₂₄H₁₅F₂N₅O₂ [M+H]⁺, 444.12. found 444.10. ¹H NMR (400 MHz, Chloroform-d) δ 9.15 (s, 1H), 8.88 (s, 2H), 8.48-8.41 (m, 1H), 7.74 (d, J=8.4 Hz, 1H), 7.65 (s, 1H), 7.51-7.38 (m, 3H), 7.31 (d, J=6.4 Hz, 1H), 6.87 (t, J=72.8 Hz, 1H), 6.36 (d, J=7.2 Hz, 1H), 5.00 (t, J=6.4 Hz, 1H), 3.55-3.44 (m, 1H), 2.89 (d, J=13.2 Hz, 1H). ¹⁹F NMR (377 MHz, Chloroform-d) δ −80.23, −80.67, −80.96, −81.41.

### Example 4: (1R,11R)-18-(difluoromethoxy)-12-methyl-5-[2-(pyrimidin-5-yl)ethynyl]-2,9,12-triazapentacyclo[9.8.1.0\hat{ }{12,10}.0\hat{ }{13,8}.0\hat{ }{14,19}]icosa-3(8),4,6,9,14(19),15,17-heptaen-13-one

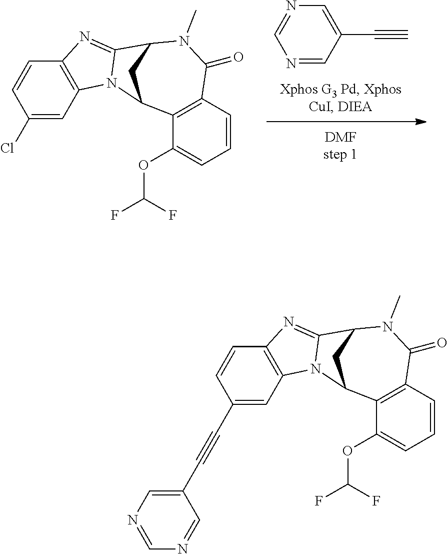

To a stirred solution of (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.385 mmol) and 5-ethynylpyrimidine (200 mg, 1.925 mmol) in DMF (3 mL) were added XPhos (18 mg, 0.039 mmol), DIPEA (0.4 mL, 1.925 mmol), CuI (3.66 mg, 0.019 mmol) and XPhos Pd G₃ (32 mg, 0.039 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm to afford (1R,11R)-18-(difluoromethoxy)-12-methyl-5-[2-(pyrimidin-5-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (20 mg, 10%) as a light grey solid. MS ESI calculated for C₂₅H₁₇F₂N₅O₂ [M+H]⁺, 458.14. found 458.05. ¹H NMR (400 MHz, Chloroform-d) δ 9.18 (s, 1H), 8.91 (s, 2H), 8.61-8.42 (m, 1H), 8.00-7.57 (m, 2H), 7.50-7.41 (m, 2H), 7.35 (d, J=8.4 Hz, 1H), 6.86 (t, J=72.8 Hz, 1H), 6.27 (s, 1H), 4.98 (s, 1H), 3.79-3.33 (m, 4H), 2.88 (d, J=13.2 Hz, 1H). ¹⁹F NMR (376 MHz, Chloroform-d) δ −80.03, −80.48, −80.93, −81.38.

### Example 5: (1R,11R)-18-(difluoromethoxy)-5-{2-[1-(2-hydroxyethyl)pyrazol-4-yl]ethynyl)}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

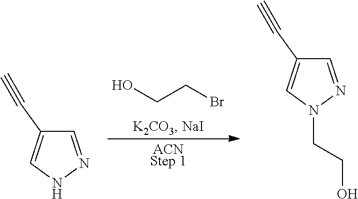

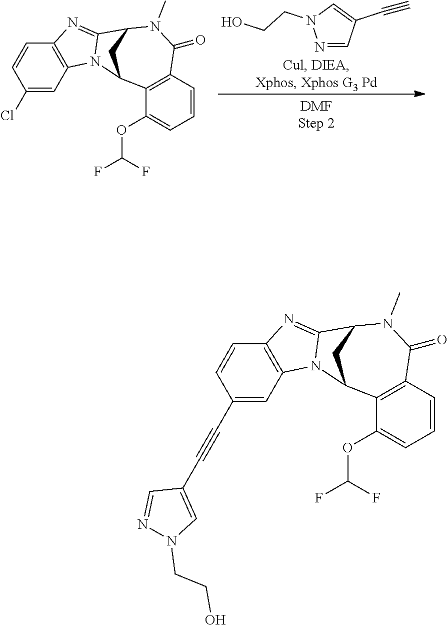

### Preparation 5A: 2-(4-ethynylpyrazol-1-yl)ethanol

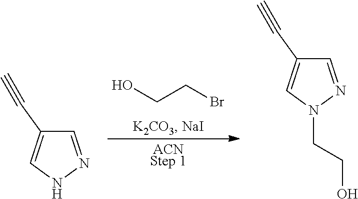

A mixture of 4-ethynyl-1H-pyrazole (1.00 g, 10.858 mmol), K₂CO₃ (4.50 g, 32.574 mmol), NaI (162 mg, 1.086 mmol) and 2-bromoethanol (2.71 g, 21.716 mmol) in ACN (15 mL) was stirred for overnight at 85° C. under nitrogen atmosphere. The mixture was allowed to cool down to room temperature. The resulting mixture was filtered, and the filter cake was washed with DCM (2×15 mL). The filtrate was concentrated under reduced pressure. The residue was purified by silica gel column chromatography, eluted with CH₂Cl₂/MeOH (10/1) to afford 2-(4-ethynylpyrazol-1-yl)ethanol (1.10 g, 74%) as a light yellow oil. MS ESI calculated for C₇H₈N₂O [M+H]⁺, 137.06. found 137.10. ¹H NMR (400 MHz, Chloroform-d) δ 7.64 (s, 1H), 7.62 (s, 1H), 4.29-4.18 (m, 2H), 4.04-3.94 (m, 2H), 3.02 (s, 1H).

### Example 5: (1R,11R)-18-(difluoromethoxy)-5-{2-[1-(2-hydroxyethyl)pyrazol-4-yl]ethynyl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

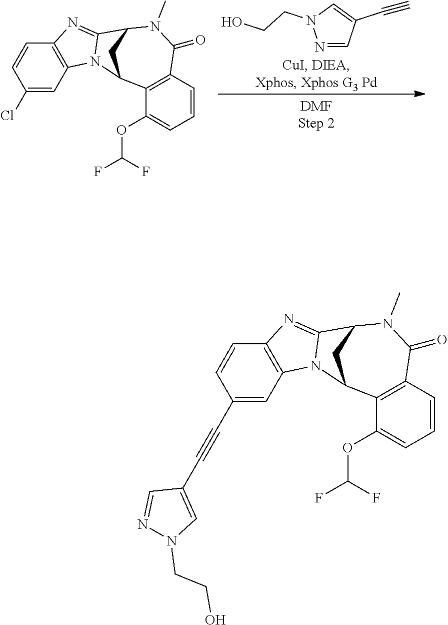

To a stirred solution of (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.385 mmol), CuI (4 mg, 0.019 mmol) and 2-(4-ethynylpyrazol-1-yl)ethanol (157 mg, 1.155 mmol) in DMF (3 mL) were added Xphos (18 mg, 0.039 mmol), DIPEA (0.4 mL, 1.925 mmol) and Xphos Pd G₃ (32 mg, 0.039 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm followed by reversed-phase flash chromatography with the following conditions: column, C18 silica gel; mobile phase, CH₃CN in water (10 mmol/L NH₄HCO₃), 25% to 45% gradient in 20 min; detector, 254 nm to afford (1R,11R)-18-(difluoromethoxy)-5-{2-[1-(2-hydroxyethyl)pyrazol-4-yl]ethynyl}-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (26 mg, 13%) as a light yellow solid. MS ESI calculated for C₂₆H₂₁F₂N₅O₃ [M+H]⁺ 490.16. found 490.25. ¹H NMR (400 MHz, DMSO-d₆) δ 8.30-8.25 (m, 1H), 8.06 (s, 1H), 7.79-7.38 (m, 6H), 7.31-7.25 (m, 1H), 6.24 (d, J=7.1 Hz, 1H), 5.24 (d, J=7.1 Hz, 1H), 4.92 (t, J=5.3 Hz, 1H), 4.16 (t, J=5.6 Hz, 2H), 3.74 (q, J=5.5 Hz, 2H), 3.55-3.45 (m, 1H), 3.34 (s, 3H), 2.81 (d, J=13.8 Hz, 1H). ¹⁹F NMR (377 MHz, DMSO-d₆) δ −81.49, −81.93, −82.02, −82.46.

### Example 6: (1R,11R)-18-(difluoromethoxy)-5-{2-[1-(2-hydroxyethyl)pyrazol-4-yl]ethynyl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

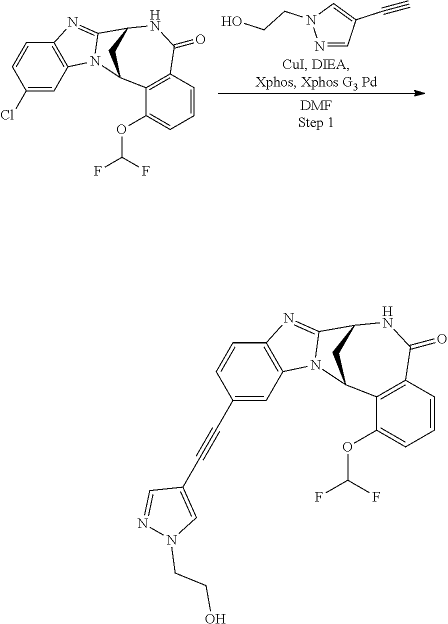

To a stirred solution of (1R,11R)-5-chloro-18-(difluoromethoxy)-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.399 mmol) and 2-(4-ethynylpyrazol-1-yl)ethanol (163.05 mg, 1.197 mmol) in DMF (3 mL) were added CuI (4 mg, 0.020 mmol), Xphos (19 mg, 0.040 mmol), Xphos Pd G3 (34 mg, 0.040 mmol) and DIPEA (0.4 mL, 1.995 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm followed by reversed-phase flash chromatography with the following conditions: column, C18 silica gel; mobile phase, CH₃CN in water (10 mmol/L NH₄HCO₃), 25% to 45% gradient in 20 min; detector, 254 nm to afford (1R,11R)-18-(difluoromethoxy)-5-{2-[1-(2-hydroxyethyl)pyrazol-4-yl]ethynyl}-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (25 mg, 12%) as a light yellow solid. MS ESI calculated for C₂₅H₁₉F₂N₅O₃ [M+H]⁺ 476.15. found 476.25. ¹H NMR (400 MHz, DMSO-d₆) δ 9.14 (d, J=6.8 Hz, 1H), 8.29-7.19 (m, 1H), 8.06 (s, 1H), 7.69 (s, 1H), 7.60 (d, J=8.4 Hz, 1H), 7.58 (t, J=72 Hz, 1H), 7.57-7.46 (m, 3H), 7.29-7.24 (m, 1H), 6.30 (d, J=7.1 Hz, 1H), 4.92 (t, J=5.2 Hz, 1H), 4.87 (t, J=6.8 Hz, 1H), 4.16 (t, J=5.6 Hz, 2H), 3.78-3.69 (m, 2H), 3.52-3.41 (m, 1H), 2.72 (d, J=13.2 Hz, 1H). ¹⁹F NMR (377 MHz, DMSO-d₆) δ −81.57, −82.02, −82.10, −82.54.

### Example 7: (1R,11R)-18-(difluoromethoxy)-12-ethyl-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

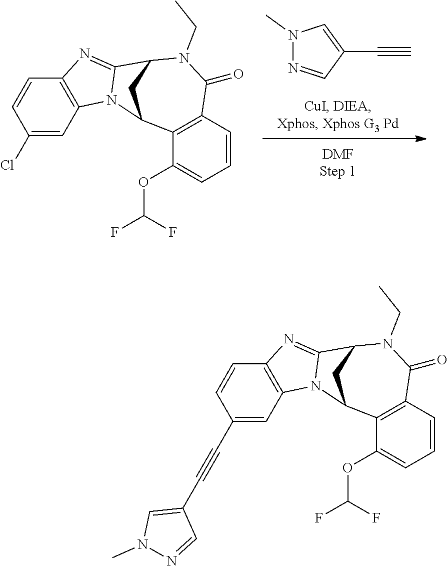

To a stirred solution of (1R,11R)-5-chloro-18-(difluoromethoxy)-12-ethyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.371 mmol) and 4-ethynyl-1-methylpyrazole (197 mg, 1.855 mmol) in DMF (3 mL) were added CuI (3 mg, 0.019 mmol), Xphos (17 mg, 0.037 mmol), Xphos Pd G₃ (31 mg, 0.037 mmol) and DIEA (240 mg, 1.855 mmol) at room temperature under nitrogen atmosphere. The resulting mixture was stirred for 16 h at 110° C. under nitrogen atmosphere. The resulting mixture was concentrated under reduced pressure. The residue was purified by silica gel column chromatography, eluted with CH₂Cl₂/MeOH (10:1) followed by reversed-phase flash chromatography with the following conditions: column, C18 silica gel; mobile phase, CH₃CN in water (10 mmol/L NH₄HCO₃), 30% to 60% gradient in 25 min; detector, 254 nm. This resulted in (1R,11R)-18-(difluoromethoxy)-12-ethyl-5-[2-(1-methylpyrazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (22 mg, 12%) as a white solid. MS ESI calculated for C₂₆H₂₁F₂N₅O₂ [M+H]⁺, 474.17. found 474.10. ¹H NMR (400 MHz, DMSO-d₆) δ 8.37-8.30 (m, 1H), 8.07 (s, 1H), 7.82-7.38 (m, 6H), 7.32-7.26 (m, 1H), 6.26 (d, J=7.0 Hz, 1H), 5.29 (d, J=7.2 Hz, 1H), 3.87 (s, 3H), 3.85-3.75 (m, 2H), 3.59-3.48 (m, 1H), 2.78 (d, J=13.7 Hz, 1H), 1.35 (t, J=7.0 Hz, 3H). ¹⁹F NMR (377 MHz, DMSO-d₆) δ −81.56, −82.01, −82.09, −82.53.

### Example 8: (7R,14R)-1-(difluoromethoxy)-6-(methyl-d3)-11-((1-methyl-1H-pyrazol-4-yl)ethynyl)-6,7-dihydro-7,14-methanobenzo[f]benzo[4,5]imidazo[1,2-a][1,4]diazocin-5(14H)-one

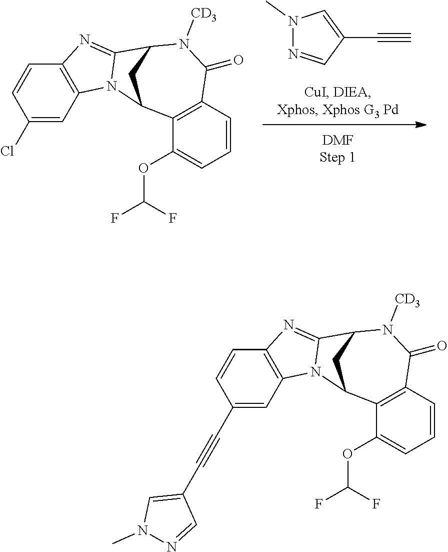

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol) and 4-ethynyl-1-methylpyrazole (203 mg, 1.910 mmol) in DMF (3 mL) were added Xphos (18 mg, 0.038 mmol), DIPEA (0.33 mL, 1.910 mmol) and Xphos Pd G₃ (32 mg, 0.038 mmol), CuI (4 mg, 0.019 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 15% to 45% gradient in 25 min; detector, 254 nm to afford (7R,14R)-1-(difluoromethoxy)-6-(methyl-d3)-11-((1-methyl-1H-pyrazol-4-yl)ethynyl)-6,7-dihydro-7,14-methanobenzo[f]benzo[4,5]imidazo[1,2-a][1,4]diazocin-5(14H)-one (14 mg, 8%) as a light yellow solid. MS ESI calculated for C₂₅H₁₆D₃F₂N₅O₂ [M+H]⁺, 463.17. found 463.05. ¹H NMR (400 MHz, Chloroform-d) δ 8.51-8.47 (m, 1H), 7.69-7.62 (m, 2H), 7.56 (d, J=1.9 Hz, 2H), 7.42 (t, J=8.2 Hz, 1H), 7.38-7.32 (m, 2H), 6.84 (t, J=73.3 Hz, 1H), 6.22 (d, J=7.2 Hz, 1H), 4.95 (d, J=7.1 Hz, 1H), 3.92 (s, 3H), 3.51-3.41 (m, 1H), 2.86 (d, J=13.5 Hz, 1H). ¹⁹F NMR (377 MHz, Chloroform-d) δ −79.80, −80.25, −81.10, −81.54.

### Example 9: (1R,11R)-18-(difluoromethoxy)-12-methyl-5-[2-(3-methylimidazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one

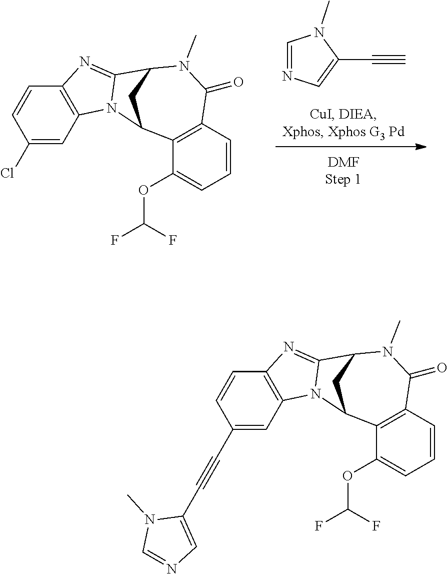

To a stirred solution of (1R,11R)-5-chloro-18-(difluoromethoxy)-12-methyl-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (150 mg, 0.385 mmol), CuI (4 mg, 0.019 mmol) and 5-ethynyl-1-methylimidazole (123 mg, 1.155 mmol) in DMF (3 mL) were added Xphos (18 mg, 0.039 mmol), Xphos Pd G₃ (33 mg, 0.039 mmol) and DIPEA (249 mg, 1.925 mmol) at room temperature under nitrogen atmosphere. The resulting mixture was stirred for overnight at 110° C. under nitrogen atmosphere. The mixture was allowed to cool down to room temperature. The mixture was purified by reversed-phase flash chromatography with the following conditions: column, C18 silica gel; mobile phase, CH₃CN in Water (0.1% FA), 15% to 45% gradient in 25 min; detector, 254 nm followed by reversed-phase flash chromatography with the following conditions: column, C18 silica gel; mobile phase, CH₃CN in water (10 mmol/L NH₄HCO₃), 25% to 45% gradient in 20 min; detector, 254 nm to afford (1R,11R)-18-(difluoromethoxy)-12-methyl-5-[2-(3-methylimidazol-4-yl)ethynyl]-2,9,12-triazapentacyclo[9.8.1.0\hat{ }{2,10}.0\hat{ }{3,8}.0\hat{ }{14,19}]icosa-3(8),4,6,9,14(19),15,17-heptaen-13-one (35 mg, 19%) as a light white solid. MS ESI calculated for C₂₅H₁₉F₂N₅O₂ [M+H]⁺, 460.15. found 460.05. ¹H NMR (400 MHz, Chloroform-d) δ 8.55-8.46 (m, 1H), 7.68 (d, J=8.4 Hz, 1H), 7.62 (d, J=1.6 Hz, 1H), 7.55 (s, 1H), 7.48-7.30 (m, 4H), 6.85 (t, J=72.8 Hz, 1H), 6.23 (d, J=7.2 Hz, 1H), 4.96 (d, J=7.2 Hz, 1H), 3.76 (s, 3H), 3.51 (s, 3H), 3.50-3.41 (m, 1H), 2.88 (d, J=13.6 Hz, 1H). ¹⁹F NMR (376 MHz, Chloroform-d) δ −80.14, −80.59, −80.91, −81.35.

### Example 10: (7R,14R)-1-(difluoromethoxy)-11-((1-(2-hydroxyethyl)-1H-pyrazol-4-yl)ethynyl)-6-(methyl-d3)-6,7-dihydro-7,14-methanobenzo[f]benzo[4,5]imidazo[1,2-a][1,4]diazocin-5(14H)-one

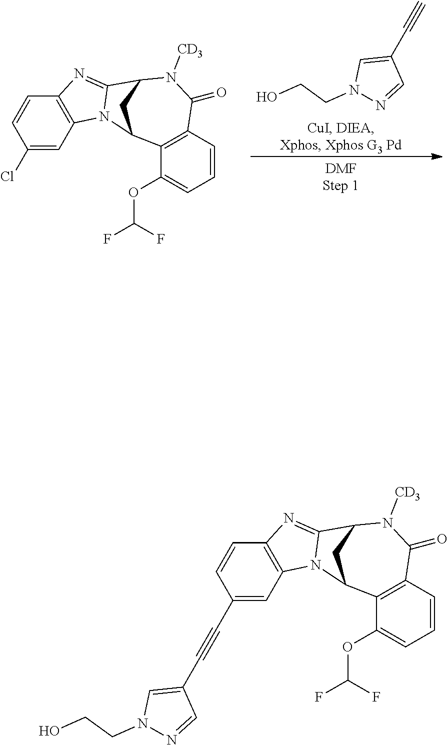

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol) and 2-(4-ethynylpyrazol-1-yl)ethanol (260 mg, 1.910 mmol) in DMF (3 mL) were added Xphos (18 mg, 0.038 mmol), CuI (3.64 mg, 0.019 mmol), DIEA (247 mg, 1.910 mmol) and Xphos Pd G₃ (32 mg, 0.038 mmol) at room temperature under nitrogen atmosphere. The resulting mixture was stirred for 24 h at 110° C. under nitrogen atmosphere. The mixture was allowed to cool down to room temperature. The mixture was purified by reversed-phase flash chromatography with the following conditions: column, C18 silica gel; mobile phase, CH₃CN in water (0.1% FA), 20% to 45% gradient in 25 min; detector, 254 nm followed by reversed-phase flash chromatography with the following conditions: column, C18 silica gel; mobile phase, CH₃CN in Water (10 mmol/L NH₄HCO₃), 25% to 45% gradient in 30 min; detector, 254 nm to afford (7R,14R)-1-(difluoromethoxy)-11-((1-(2-hydroxyethyl)-1H-pyrazol-4-yl)ethynyl)-6-(methyl-d3)-6,7-dihydro-7,14-methanobenzo[f]benzo[4,5]imidazo[1,2-a][1,4]diazocin-5(14H)-one (29 mg, 15%) as a yellow solid. MS ESI calculated for C₂₆H₁₈D₃F₂N₅O₃ [M+H]⁺, 493.18. found 493.10. ¹H NMR (400 MHz, Chloroform-d) δ 8.52-8.47 (m, 1H), 7.72-7.62 (m, 3H), 7.57 (t, J=1.0 Hz, 1H), 7.43 (t, J=8.2 Hz, 1H), 7.39-7.32 (m, 2H), 6.84 (t, J=73.2 Hz, 1H), 6.23 (d, J=7.2 Hz, 1H), 4.98 (d, J=7.1 Hz, 1H), 4.30-4.23 (m, 2H), 4.07-4.00 (m, 2H), 3.50-3.41 (m, 1H), 2.87 (d, J=13.6 Hz, 1H). ¹⁹F NMR (377 MHz, Chloroform-d) δ −79.85, −80.29, −81.06, −81.50.

### Example 11: (7R,14R)-1-(difluoromethoxy)-6-(methyl-d3)-11-(pyrimidin-5-ylethynyl)-6,7-dihydro-7,14-methanobenzo[f]benzo[4,5]imidazo[1,2-a][1,4]diazocin-5(14H)-one

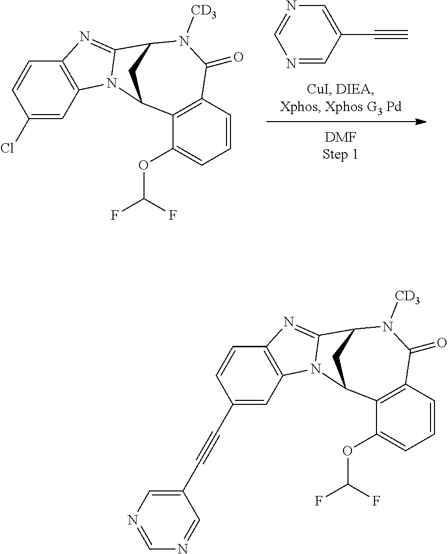

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol), CuI (4 mg, 0.019 mmol), Xphos (18 mg, 0.038 mmol) and 5-ethynylpyrimidine (119 mg, 1.146 mmol) in DMF (2 mL) were added DIEA (247 mg, 1.910 mmol) and Xphos Pd G₃ (32 mg, 0.038 mmol). After stirring for 16 h at 110° C. under a nitrogen atmosphere, the resulting mixture was concentrated under reduced pressure. The resulting mixture was filtered, and the filter cake was washed with MeOH (3×5 mL). The filtrate was concentrated under reduced pressure. The residue was purified by reversed-phase flash chromatography with the following conditions: column, C18 silica gel; mobile phase, CH₃CN in water (10 mmol/L NH₄HCO₃), 20% to 50% gradient in 30 min; detector, 254 nm. This resulted in (7R,14R)-1-(difluoromethoxy)-6-(methyl-d3)-11-(pyrimidin-5-ylethynyl)-6,7-dihydro-7,14-methanobenzo[f]benzo[4,5]imidazo[1,2-a][1,4]diazocin-5(14H)-one (13 mg, 7%) as a white solid. MS ESI calculated for C₂₅H₁₄D₃F₂N₅O₂ [M+H]⁺, 461.15. found 461.10. ¹H NMR (400 MHz, Chloroform-d) δ 9.15 (s, 1H), 8.87 (s, 2H), 8.53-8.48 (m, 1H), 7.73 (d, J=8.4 Hz, 1H), 7.68 (s, 1H), 7.51-7.41 (m, 2H), 7.36 (d, J=8.1 Hz, 1H), 6.87 (t, J=72.7 Hz, 1H), 6.32-6.24 (m, 1H), 5.07-4.98 (m, 1H), 3.57-3.42 (m, 1H), 2.90 (d, J=9.8 Hz, 1H). ¹⁹F NMR (377 MHz, Chloroform-d) δ −80.10, −80.55, −80.95, −81.39.

### Example 12: (7R,14R)-1-(difluoromethoxy)-6-(methyl-d3)-11-((1-methyl-1H-1,2,3-triazol-5-yl)ethynyl)-6,7-dihydro-7,14-methanobenzo[f]benzo[4,5]imidazo[1,2-a][1,4]diazocin-5(14H)-one

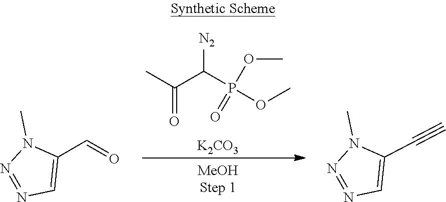

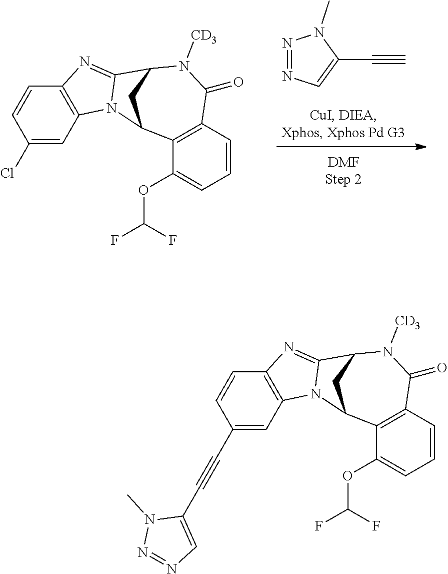

### Preparation 12A: 5-ethynyl-1-methyl-1,2,3-triazole

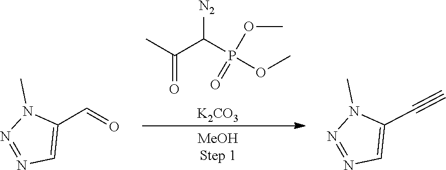

To a stirred mixture of 3-methyl-1,2,3-triazole-4-carbaldehyde (900 mg, 8.101 mmol) and K₂CO₃ (2.24 g, 16.202 mmol) in MeOH (20 mL) was added seyferth-gilbert homologation (1.72 g, 8.911 mmol) dropwise at 0° C. under nitrogen atmosphere. The mixture was stirred for 3 h at 25° C. The resulting mixture was concentrated under reduced pressure. The residue was purified by silica gel column chromatography, eluted with DCM/MeOH (20/1) to afford 5-ethynyl-1-methyl-1,2,3-triazole (732 mg, 84%) as a light yellow solid. MS ESI calculated for C₅H₅N [M+H]⁺, 108.05. found 108.20. ¹H NMR (400 MHz, Chloroform-d) δ 7.81 (s, 1H), 4.11 (s, 3H), 3.70 (s, 1H).

### Example 12: (7R,14R)-1-(difluoromethoxy)-6-(methyl-d3)-11-((1-methyl-1H-1,2,3-triazol-5-yl)ethynyl)-6,7-dihydro-7,14-methanobenzo[f]benzo[4,5]imidazo[1,2-a][1,4]diazocin-5(14H)-one

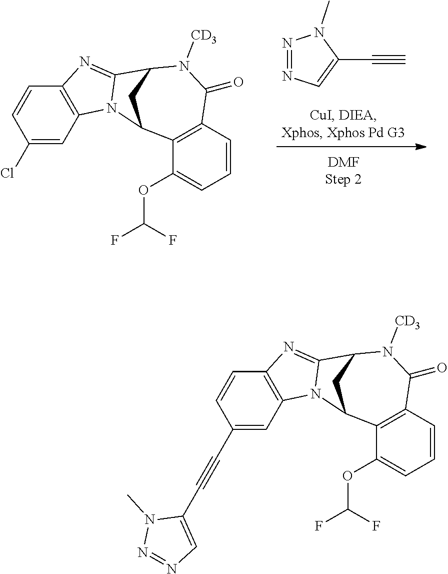

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (130 mg, 0.331 mmol), CuI (3 mg, 0.017 mmol) and 5-ethynyl-1-methyl-1,2,3-triazole (177 mg, 1.655 mmol) in DMF (3 mL) were added XPhos (32 mg, 0.066 mmol), XPhos Pd G₃ (56 mg, 0.066 mmol) and DIPEA (289 uL, 1.655 mmol) at room temperature under nitrogen atmosphere. The resulting mixture was stirred for overnight at 110° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 25% B to 42% B in 10 min; Wave Length: 254 nm/220 nm to afford (7R,14R)-1-(difluoromethoxy)-6-(methyl-d3)-11-((1-methyl-1H-1,2,3-triazol-5-yl)ethynyl)-6,7-dihydro-7,14-methanobenzo[f]benzo[4,5]imidazo[1,2-a][1,4]diazocin-5(14H)-one (26 mg, 16%) as a white solid. MS ESI calculated for C₂₄H₁₅D₃F₂N₆O₂[M+H]⁺, 464.16. found 464.10. ¹H NMR (400 MHz, Chloroform-d) δ 8.53-8.47 (m, 1H), 7.84 (s, 1H), 7.74 (d, J=8.4 Hz, 1H), 7.72-7.67 (m, 1H), 7.49-7.40 (m, 2H), 7.37-7.31 (m, 1H), 6.87 (t, J=72.8 Hz, 1H), 6.28 (d, J=6.4 Hz, 1H), 5.04 (d, J=6.0 Hz, 1H), 4.16 (s, 3H), 3.56-3.42 (m, 1H), 2.91 (d, J=13.2 Hz, 1H). ¹⁹F NMR (377 MHz, Chloroform-d) δ −80.31, −80.76, −80.80, −81.24.

### Example 13: (7R,14R)-1-(difluoromethoxy)-6-(methyl-d3)-11-((1-methyl-1H-pyrazol-5-yl)ethynyl)-6,7-dihydro-7,14-methanobenzo[f]benzo[4,5]imidazo[1,2-a][1,4]diazocin-5(14H)-one

### Synthetic Scheme

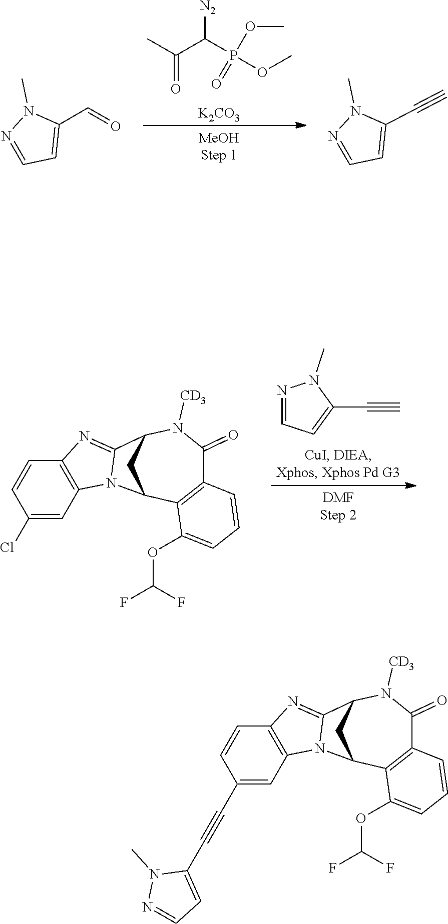

### Preparation 13A: 5-ethynyl-1-methylpyrazole

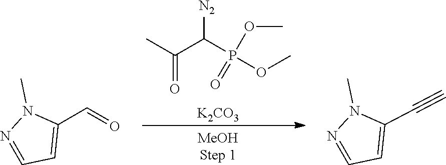

To a stirred mixture of 2-methylpyrazole-3-carbaldehyde (5.00 g, 45.407 mmol) and K₂CO₃ (12.55 g, 90.814 mmol) in MeOH (50 mL) was added seyferth-gilbert homologation (9.60 g, 49.948 mmol) dropwise at 0° C. under nitrogen atmosphere. The mixture was stirred for 3 h at 25° C. The resulting mixture was concentrated under reduced pressure. The residue was purified by silica gel column chromatography, eluted with PE/EA (3/1) to afford 5-ethynyl-1-methylpyrazole (2.96 g, 61%) as a light yellow oil. MS ESI calculated for C₆H₆N₂ [M+H]⁺, 107.05. found 107.15.

### Example 13: (7R,14R)-1-(difluoromethoxy)-6-(methyl-d3)-11-((1-methyl-1H-pyrazol-5-yl)ethynyl)-6,7-dihydro-7,14-methanobenzo[f]benzo[4,5]imidazo[1,2-a][1,4]diazocin-5(14H)-one

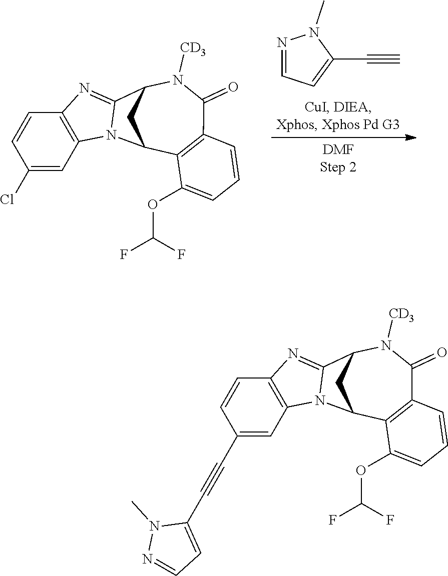

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (130 mg, 0.331 mmol), XPhos (31 mg, 0.066 mmol) and 5-ethynyl-1-methylpyrazole (140 mg, 1.324 mmol) in DMF (3 mL) were added XPhos Pd G3 (56 mg, 0.066 mmol), CuI (3 mg, 0.017 mmol) and DIPEA (288 uL, 1.655 mmol) at room temperature under nitrogen atmosphere. The resulting mixture was stirred for overnight at 110° C. under nitrogen atmosphere. The resulting mixture was diluted with water (20 mL) and extracted with EtOAc (3×20 mL).The combined organic layers were washed with brine (40 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((1-methyl-1H-pyrazol-5-yl)ethynyl)-6,7-dihydro-7,14-methanobenzo[f]benzo[4,5]imidazo[1,2-a][1,4]diazocin-5(14H)-one (25 mg, 15%) as a light yellow solid. MS ESI calculated for C₂₅H₁₆D₃F₂N₅O₂ [M+H]⁺, 463.17. found 463.05. ¹H NMR (400 MHz, Chloroform-d) δ 8.52-8.47 (m, 1H), 7.72 (d, J=8.4 Hz, 1H), 7.69-7.65 (m, 1H), 7.49-7.41 (m, 3H), 7.34 (d, J=8.0 Hz, 1H), 6.86 (t, J=72.8 Hz, 1H), 6.50 (d, J=2.0 Hz, 1H), 6.28 (d, J=6.8 Hz, 1H), 5.05 (d, J=6.8 Hz, 1H), 4.01 (s, 3H), 3.56-3.42 (m, 1H), 2.90 (d, J=13.6 Hz, 1H). ¹⁹F NMR (377 MHz, Chloroform-d) δ −80.27, −80.72, −80.94, −81.38.

### Example 14: (7R,14R)-11-((1H-pyrazol-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

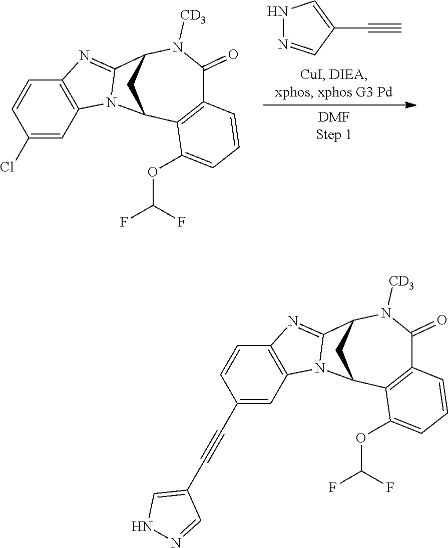

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (130 mg, 0.331 mmol), XPhos (32 mg, 0.066 mmol) and 4-ethynyl-1H-pyrazole (153 mg, 1.655 mmol) in DMF (3 mL) were added CuI (3 mg, 0.017 mmol), XPhos Pd G3 (32 mg, 0.066 mmol) and DIPEA (289 uL, 1.655 mmol) at room temperature under nitrogen atmosphere. The resulting mixture was stirred for overnight at 110° C. under nitrogen atmosphere. The resulting mixture was diluted with water (20 mL) and extracted with EtOAc (3×20 mL). The combined organic layers were washed with brine (40 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-11-((1H-pyrazol-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (24 mg, 16%) as a light brown solid. MS ESI calculated for C₂₄H₁₄D₃F₂N₅O₂ [M+H]⁺, 449.15. found 449.05. ¹H NMR (400 MHz, DMSO-d₆) δ 13.18 (s, 1H), 8.31-8.26 (m, 1H), 8.12 (s, 1H), 7.73 (s, 1H), 7.63 (d, J=8.4 Hz, 1H), 7.58 (t, J=72.4 Hz, 1H), 7.55-7.47 (m, 3H), 7.31-7.26 (m, 1H), 6.24 (d, J=7.2 Hz, 1H), 5.23 (d, J=7.2 Hz, 1H), 3.56-3.45 (m, 1H), 2.81 (d, J=13.6 Hz, 1H). ¹⁹F NMR (377 MHz, DMSO-d₆) δ −81.46, −81.90, −81.98, −82.43.

### Example 15: (7R,14R)-11-((1-(2-aminoethyl)-1H-pyrazol-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

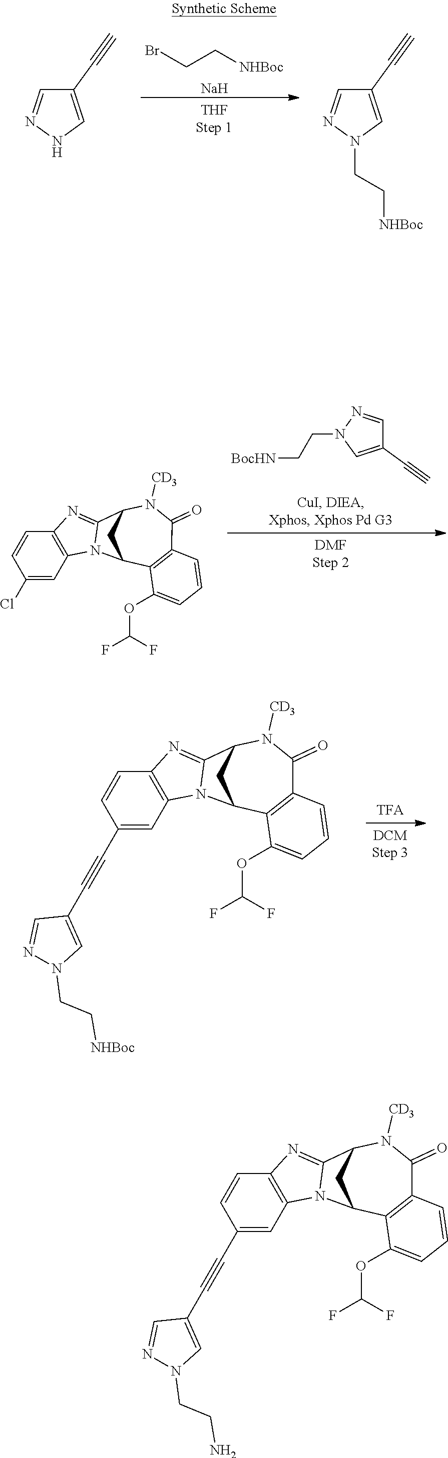

### Preparation 15A: tert-butyl N-[2-(4-ethynylpyrazol-1-yl)ethyl]carbamate

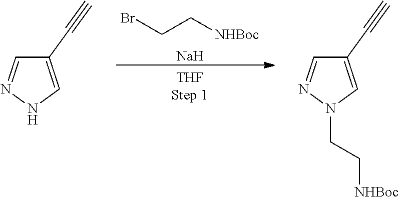

To a stirred solution of 4-ethynyl-1H-pyrazole (1.00 g, 10.858 mmol) in THF (10 mL) was added NaH (0.65 g, 16.287 mmol, 60%) at 0° C. under nitrogen atmosphere. The resulting solution was stirred for 30 min at 0° C. under nitrogen atmosphere. To the above solution was added tert-butyl N-(2-bromoethyl)carbamate (2.92 g, 13.030 mmol) at 0° C. under nitrogen atmosphere. The resulting mixture was stirred for 16 h at room temperature under nitrogen atmosphere. The reaction was quenched with water (1 mL) at 0° C. The resulting mixture was concentrated under reduced pressure. The residue was purified by silica gel column chromatography, eluted with PE/EA (6/4) to afford tert-butyl N-[2-(4-ethynylpyrazol-1-yl)ethyl]carbamate (1.31 g, 51%) as a white solid. MS ESI calculated for C₁₂H₁₇N₃O₂ [M+H]⁺, 236.13. found 236.00. ¹H NMR (400 MHz, Chloroform-d) δ 7.64 (s, 1H), 7.54 (s, 1H), 4.21 (t, J=5.7 Hz, 2H), 3.58-3.53 (m, 2H), 3.01 (s, 1H), 1.44 (s, 9H).

### Preparation 15B: tert-butyl (2-(4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1H-pyrazol-1-yl)ethyl)carbamate

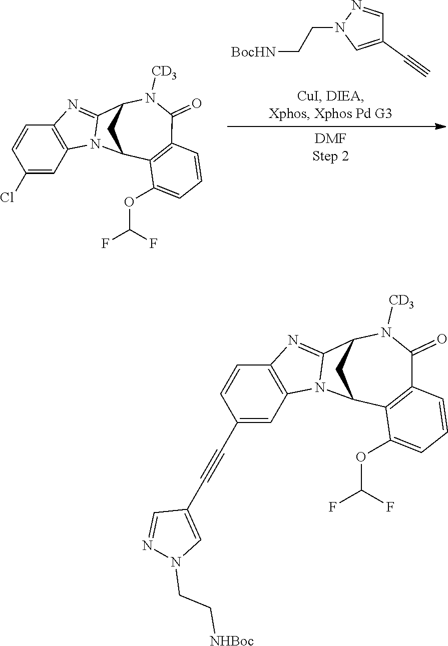 [SMILES: [2H]C([2H])([2H])N1C(=O)c2cccc(OC(F)F)c2[C@H]2C[C@@H]1c1nc3ccc(Cl)cc3n12 ; C#Cc1cnn(CCNC(=O)OC(C)(C)C)c1 ; [2H]C([2H])([2H])N1C(=O)c2cccc(OC(F)F)c2[C@H]2C[C@@H]1c1nc3ccc(C#Cc4cnn(CCNC(=O)OC(C)(C)C)c4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (200 mg, 0.509 mmol) and tert-butyl N-[2-(4-ethynylpyrazol-1-yl)ethyl]carbamate (356 mg, 1.527 mmol) in DMF (3 mL) were added XPhos (24 mg, 0.051 mmol), CuI (5 mg, 0.025 mmol), DIEA (329 mg, 2.545 mmol) and XPhos Pd G3 (43 mg, 0.051 mmol) under nitrogen atmosphere. The resulting mixture was stirred for 24 h at 110° C. under nitrogen atmosphere. The resulting mixture was concentrated under reduced pressure. The residue was purified by silica gel column chromatography, eluted with CH₂Cl₂/MeOH (15/1) to afford tert-butyl (2-(4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1H-pyrazol-1-yl)ethyl)carbamate (93 mg, 31%) as a yellow solid. MS ESI calculated for C₃₁H₂₇D₃F₂N₆O₄ [M+H]⁺, 592.25. found 592.30.

### Example 15: (7R,14R)-11-((1-(2-aminoethyl)-1H-pyrazol-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

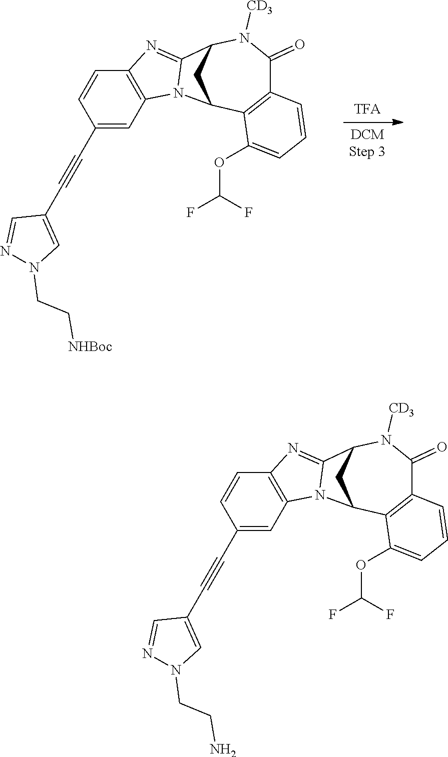 [SMILES: [2H]C([2H])([2H])N1C(=O)c2cccc(OC(F)F)c2[C@H]2C[C@@H]1c1nc3ccc(C#Cc4cnn(CCNC(=O)OC(C)(C)C)c4)cc3n12 ; [2H]C([2H])([2H])N1C(=O)c2cccc(OC(F)F)c2[C@H]2C[C@@H]1c1nc3ccc(C#Cc4cnn(CCN)c4)cc3n12]

A solution of tert-butyl (2-(4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1H-pyrazol-1-yl)ethyl)carbamate (80 mg, 0.135 mmol) in DCM (2 mL) was added TFA (0.5 mL, 6.732 mmol) dropwise at 0° C. The resulting solution was stirred for 3 h at room temperature. The resulting solution was concentrated under reduced pressure. The residue was purified by reversed-phase flash chromatography with the following conditions: column, C18 silica gel; mobile phase, CH₃CN in water (10 mmol/L NH₄HCO₃), 30% to 50% gradient in 30 min; detector, 254 nm. This resulted in (7R,14R)-11-((1-(2-aminoethyl)-1H-pyrazol-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 29%) as an off-white solid. MS ESI calculated for C₂₆H₁₉D₃F₂N₆O₂ [M+H]⁺, 492.20. found 492.10. ¹H NMR (400 MHz, Chloroform-d) δ 8.48 (d, J=8.2 Hz, 1H), 7.73-7.61 (m, 3H), 7.55 (s, 1H), 7.41 (t, J=8.1 Hz, 1H), 7.38-7.31 (m, 2H), 6.84 (t, J=72.7 Hz, 1H), 6.19 (d, J=7.3 Hz, 1H), 4.92 (d, J=7.1 Hz, 1H), 4.24 (t, J=5.7 Hz, 2H), 3.47-3.40 (m, 1H), 3.25 (t, J=6.0 Hz, 2H), 2.85 (d, J=13.6 Hz, 1H). ¹⁹F NMR (377 MHz, Chloroform-d) δ −79.82, −80.26, −81.09, −81.54.

### Example 16: (7R,14R)-1-(difluoromethoxy)-11-((2-(2-hydroxypropan-2-yl)pyrimidin-5-yl)ethynyl)-6-(methyl-d3)-6,7-dihydro-7,14-methanobenzo[f]benzo[4,5]imidazo[1,2-a][1,4]diazocin-5(14H)-one

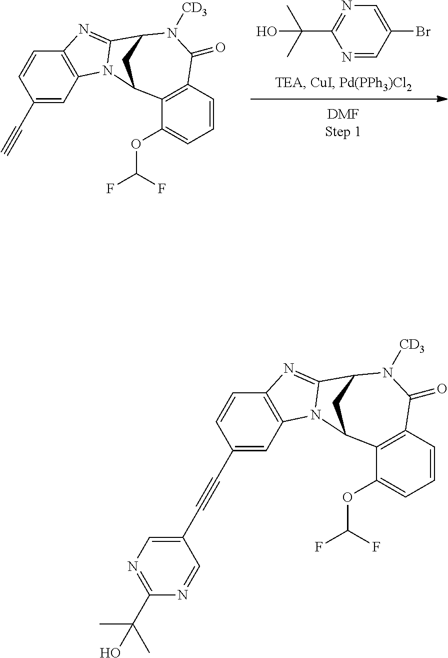

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), TEA (73 uL, 0.524 mmol) and 2-(5-bromopyrimidin-2-yl)propan-2-ol (57 mg, 0.262 mmol) in DMF (0.5 mL) were added Pd(PPh₃)₂Cl₂ (9 mg, 0.013 mmol) and CuI (1 mg, 0.007 mmol) at room temperature under nitrogen atmosphere. The resulting mixture was stirred for overnight at 80° C. under nitrogen atmosphere. The resulting mixture was concentrated under vacuum. The residue purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-((2-(2-hydroxypropan-2-yl)pyrimidin-5-yl)ethynyl)-6-(methyl-d3)-6,7-dihydro-7,14-methanobenzo[f]benzo[4,5]imidazo[1,2-a][1,4]diazocin-5(14H)-one (45 mg, 66%) as a white solid. MS ESI calculated for C₂₈H₂₀D₃F₂N₅O₃ [M+H]⁺, 519.20. found 519.20. ¹H NMR (400 MHz, Chloroform-d) δ 8.84 (s, 2H), 8.55-8.46 (m, 1H), 7.75 (d, J=8.4 Hz, 1H), 7.70 (d, J=1.2 Hz, 1H), 7.50-7.41 (m, 2H), 7.36 (d, J=8.4 Hz, 1H), 6.87 (t, J=72.8 Hz, 1H), 6.30 (d, J=6.8 Hz, 1H), 5.10 (d, J=6.8 Hz, 1H), 4.50 (br, 1H), 3.58-3.45 (m, 1H), 2.92 (d, J=13.6 Hz, 1H), 1.62 (s, 6H). ¹⁹F NMR (376 MHz, Chloroform-d) δ −80.17, −80.62, −80.91, −81.36.

### Example 17: (7R,14R)-1-(difluoromethoxy)-6-(methyl-d3)-11-((1-methyl-1H-imidazol-5-yl)ethynyl)-6,7-dihydro-7,14-methanobenzo[f]benzo[4,5]imidazo[1,2-a][1,4]diazocin-5(14H)-one

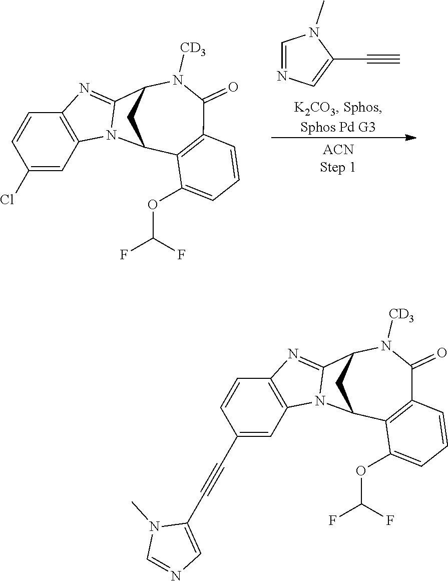

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (50 mg, 0.127 mmol), Sphos (11 mg, 0.025 mmol) and 5-ethynyl-1-methylimidazole (41 mg, 0.381 mmol) in ACN (1 mL) were added K₂CO₃ (87 mg, 0.635 mmol) and Sphos Pd G3 (20 mg, 0.025 mmol). After stirring for overnight at 90° C. under a nitrogen atmosphere, the resulting mixture was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((1-methyl-1H-imidazol-5-yl)ethynyl)-6,7-dihydro-7,14-methanobenzo[f]benzo[4,5]imidazo[1,2-a][1,4]diazocin-5(14H)-one (24 mg, 40%) as a white solid. MS ESI calculated for C₂₅H₁₆D₃F₂N₅O₂ [M+H]⁺, 463.17. found 463.15. ¹H NMR (400 MHz, Chloroform-d) δ 8.53-8.44 (m, 1H), 7.68 (d, J=8.4 Hz, 1H), 7.63 (d, J=1.6 Hz, 1H), 7.58 (s, 1H), 7.47-7.30 (m, 4H), 6.85 (t, J=72.8 Hz, 1H), 6.23 (d, J=7.2 Hz, 1H), 4.95 (d, J=7.2 Hz, 1H), 3.76 (s, 3H), 3.52-3.40 (m, 1H), 2.87 (d, J=13.6 Hz, 1H). ¹⁹F NMR (377 MHz, Chloroform-d) δ −80.16, −80.61, −80.91, −81.36.

### Example 18: (7R,14R)-1-(difluoromethoxy)-6-(methyl-d3)-11-((1-methyl-1H-1,2,3-triazol-4-yl)ethynyl)-6,7-dihydro-7,14-methanobenzo[f]benzo[4,5]imidazo[1,2-a][1,4]diazocin-5(14H)-one

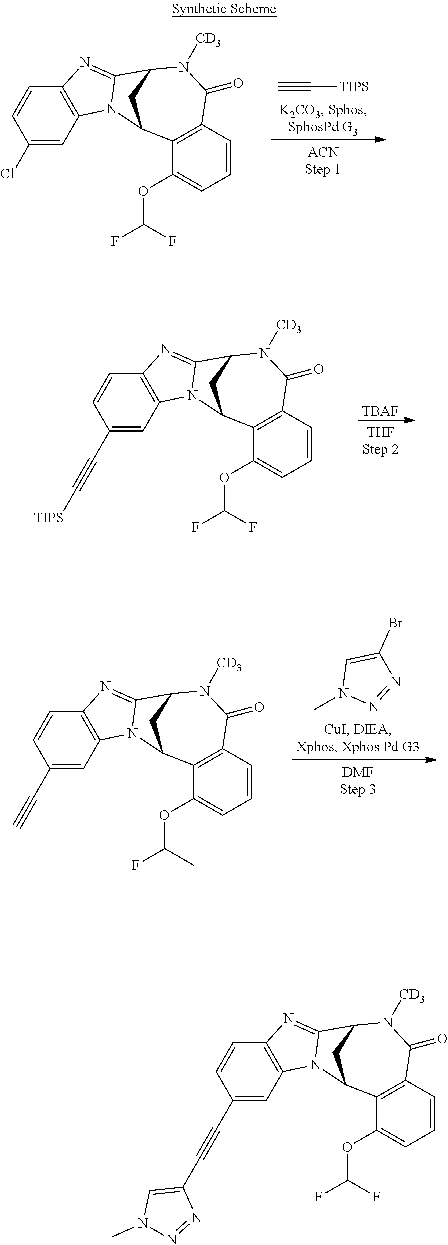

### Preparation 18A: (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one

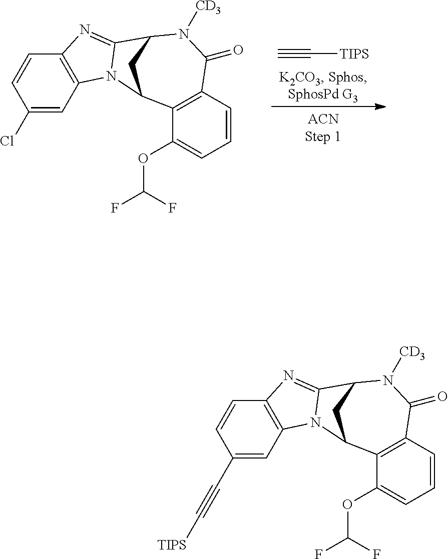

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (1.00 g, 2.546 mmol), Sphos (105 mg, 0.255 mmol) and ethynyltriisopropylsilane (2.32 g, 12.730 mmol) in ACN (15 mL) were added K₂CO₃ (1.76 g, 12.730 mmol) and Sphos Pd G3 (199 mg, 0.255 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with EA (100%) to afford (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one (1.18 g, 86%) as a light yellow solid. MS ESI calculated for C₃₀H₃₂D₃F₂N₃O₂Si [M+H]⁺, 539.27. found 539.30. ¹H NMR (400 MHz, Chloroform-d) δ 8.51-8.45 (m, 1H), 7.67-7.62 (m, 2H), 7.47-7.37 (m, 2H), 7.34-7.30 (m, 1H), 7.03-6.64 (m, 1H), 6.28 (d, J=7.2 Hz, 1H), 5.08 (d, J=7.2 Hz, 1H), 3.54-3.44 (m, 1H), 2.89 (d, J=13.6 Hz, 1H), 1.14 (s, 21H).

### Preparation 18B: (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one

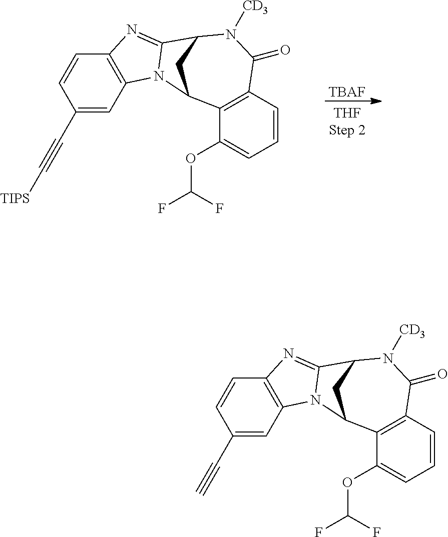

To a stirred solution of (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one (1.18 g, 2.190 mmol) in THF (20 mL) was added TBAF (2.7 mL, 2.628 mmol, 1M in THF) dropwise at 0° C. under nitrogen atmosphere. The resulting mixture was stirred for 3 h at room temperature. The resulting mixture was concentrated under vacuum. The residue was purified by silica gel column chromatography, eluted with CH₂Cl₂/MeOH (10/1) to afford ((7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (745 mg, 88%) as a light yellow solid. MS ESI calculated for C₂₁H₁₂D₃F₂N₃O₂ [M+H]⁺, 383.13. found 383.15. ¹H NMR (400 MHz, Chloroform-d) δ 8.52-8.46 (m, 1H), 7.65 (d, J=8.4 Hz, 1H), 7.61 (d, J=1.6 Hz, 1H), 7.42 (t, J=8.4 Hz, 1H), 7.39-7.31 (m, 2H), 6.83 (t, J=73.2 Hz, 1H), 6.22 (d, J=7.2 Hz, 1H), 4.96 (d, J=7.2 Hz, 1H), 3.50-3.38 (m, 1H), 3.07 (s, 1H), 2.86 (d, J=13.6 Hz, 1H). ¹⁹F NMR (377 MHz, Chloroform-d) δ −79.96, −80.40, −81.32, −81.76.

### Example 18: (7R,14R)-1-(difluoromethoxy)-6-(methyl-d3)-11-((1-methyl-1H-1,2,3-triazol-4-yl)ethynyl)-6,7-dihydro-7,14-methanobenzo[f]benzo[4,5]imidazo[1,2-a][1,4]diazocin-5(14H)-one

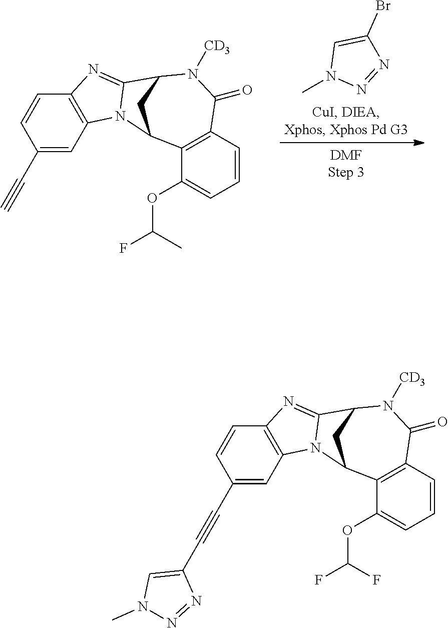

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (55 mg, 0.144 mmol), DIPEA (93 mg, 0.720 mmol) and 4-bromo-1-methyl-1,2,3-triazole (94 mg, 0.576 mmol) in DMF (2 mL) were added Xphos (7 mg, 0.014 mmol), CuI (3 mg, 0.014 mmol) and XPhos Pd G3 (13 mg, 0.014 mmol). The resulting mixture was stirred for overnight at 110° C. under nitrogen atmosphere. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (0.1% NH₄HCO₃), Mobile Phase B: CH₃CN; Flow rate: 50 mL/min; Gradient: 25% B to 60% B in 25 min; 254/220 nm to afford (7R,14R)-1-(difluoromethoxy)-6-(methyl-d3)-11-((1-methyl-1H-1,2,3-triazol-4-yl)ethynyl)-6,7-dihydro-7,14-methanobenzo[f]benzo[4,5]imidazo[1,2-a][1,4]diazocin-5(14H)-one (16 mg, 23%) as a white solid. MS ESI calculated for C₂₄H₁₅D₃F₂N₆O₂[M+H]⁺, 464.16. found 464.15. ¹H NMR (400 MHz, Chloroform-d) δ 8.52-8.44 (m, 1H), 7.76-7.56 (m, 3H), 7.46-7.32 (m, 3H), 6.86 (t, J=72.8 Hz, 1H), 6.22 (d, J=7.2 Hz, 1H), 4.94 (d, J=6.8 Hz, 1H), 4.13 (s, 3H), 3.52-3.38 (m, 1H), 2.86 (d, J=13.6 Hz, 1H). ¹⁹F NMR (377 MHz, Chloroform-d) δ −79.96, −80.40, −81.32, −81.76.

### Example 19: (7R,14R)-1-(difluoromethoxy)-11-((1-methyl-1H-1,2,3-triazol-5-yl)ethynyl)-6,7-dihydro-7,14-methanobenzo[f]benzo[4,5]imidazo[1,2-a][1,4]diazocin-5(14H)-one

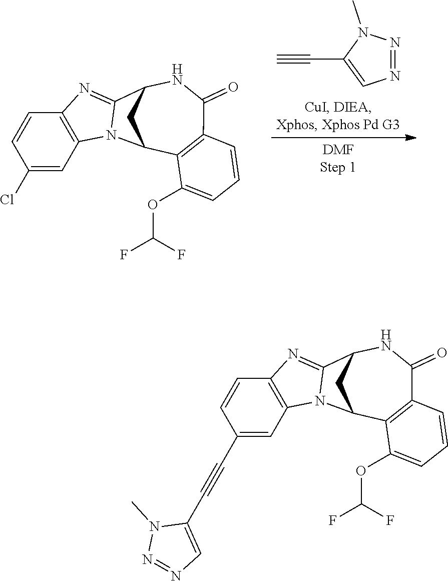

A mixture of (7R,14R)-11-chloro-1-(difluoromethoxy)-6,7-dihydro-7,14-methanobenzo[f]benzo[4,5]imidazo[1,2-a][1,4]diazocin-5(14H)-one (150 mg, 0.399 mmol), 5-ethynyl-1-methyl-1,2,3-triazole (171 mg, 1.596 mmol), Xphos (19 mg, 0.040 mmol), Xphos Pd G3 (34 mg, 0.040 mmol), CuI (4 mg, 0.020 mmol) and DIPEA (348 uL, 1.995 mmol) in DMF (3 mL) was stirred for overnight at 110° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: XBridge Shield RP18 OBD Column 30*150 mm, 5m; Mobile Phase A: water (10 mmol/L NH₄HCO₃), Mobile Phase B: ACN; Flow rate: 60 mL/min mL/min; Gradient: 22% B to 37% B in 10 min; Wave Length: 254 nm/220 nm to afford (7R,14R)-1-(difluoromethoxy)-11-((1-methyl-1H-1,2,3-triazol-5-yl)ethynyl)-6,7-dihydro-7,14-methanobenzo[f]benzo[4,5]imidazo[1,2-a][1,4]diazocin-5(14H)-one (15 mg, 8%) as a light yellow solid. MS ESI calculated for C₂₃H₁₆F₂N₆O₂[M+H]⁺, 447.13. found 447.05. ¹H NMR (400 MHz, DMSO-d₆) δ 9.16 (d, J=6.8 Hz, 1H), 8.28-8.18 (m, 1H), 8.09 (s, 1H), 7.70 (d, J=8.4 Hz, 1H), 7.65 (d, J=1.6 Hz, 1H), 7.63 (t, J=73.2 Hz, 1H), 7.57-7.48 (m, 2H), 7.47-7.41 (m, 1H), 6.34 (d, J=7.2 Hz, 1H), 4.91 (t, J=6.8 Hz, 1H), 4.14 (s, 3H), 3.47-3.40 (m, 1H), 2.75 (d, J=13.6 Hz, 1H). ¹⁹F NMR (377 MHz, DMSO-d₆) δ −81.78, −82.22, −82.38, −82.83.

### Example 20: 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methyl-1H-pyrazole-3-carbonitrile

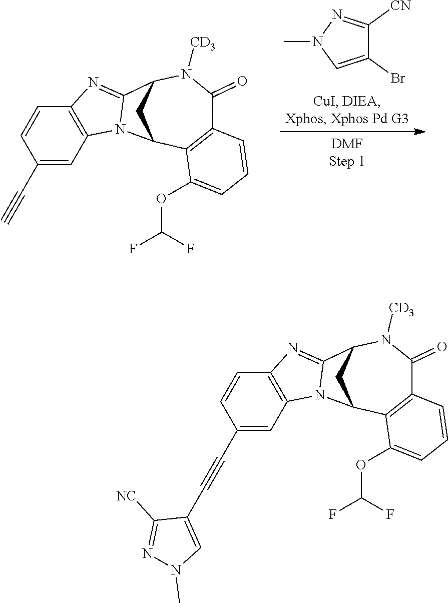

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol) and 4-bromo-1-methylpyrazole-3-carbonitrile (72 mg, 0.393 mmol) in DMF (1 mL) were added CuI (2 mg, 0.013 mmol), Xphos (6 mg, 0.013 mmol), DIPEA (84 mg, 0.655 mmol) and Xphos Pd G₃ (11 mg, 0.013 mmol) at room temperature under nitrogen atmosphere. The resulting mixture was stirred for 16 h at 80° C. under nitrogen atmosphere. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methyl-1H-pyrazole-3-carbonitrile (19 mg, 30%) as a white solid. MS ESI calculated for C₂₆H₁₅D₃F₂N₆O₂[M+H]⁺, 488.16. found 488.15. ¹H NMR (400 MHz, Chloroform-d) δ 8.49 (d, J=7.7 Hz, 1H), 7.73-7.63 (m, 2H), 7.60 (s, 1H), 7.47-7.33 (m, 3H), 7.10-6.71 (m, 1H), 6.28 (d, J=6.5 Hz, 1H), 5.04 (d, J=4.5 Hz, 1H), 3.99 (s, 3H), 3.55-3.43 (m, 1H), 2.90 (d, J=13.4 Hz, 1H). ¹⁹F NMR (377 MHz, Chloroform-d) δ −80.12, −80.56, −81.69, −82.14.

### Example 21: (7R,14R)-1-(difluoromethoxy)-6-(methyl-d3)-11-((2-methylpyrimidin-5-yl)ethynyl)-6,7-dihydro-7,14-methanobenzo[f]benzo[4,5]imidazo[1,2-a][1,4]diazocin-5(14H)-one

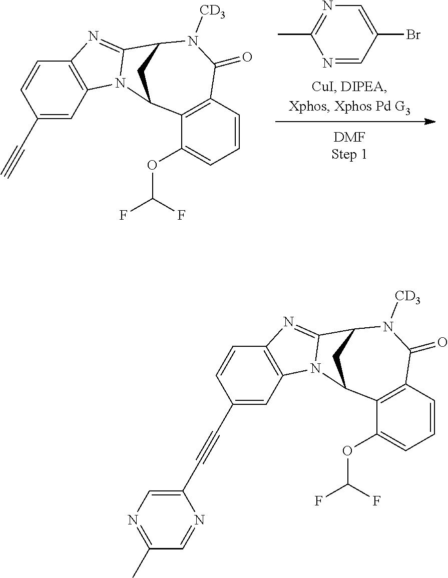

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (67 mg, 0.393 mmol) in DMF (1 mL) were added CuI (2 mg, 0.013 mmol), Xphos (6 mg, 0.013 mmol), DIPEA (84 mg, 0.655 mmol) and Xphos Pd G₃ (11 mg, 0.013 mmol) at room temperature under nitrogen atmosphere. The resulting mixture was stirred for overnight at 80° C. under nitrogen atmosphere. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((2-methylpyrimidin-5-yl)ethynyl)-6,7-dihydro-7,14-methanobenzo[f]benzo[4,5]imidazo[1,2-a][1,4]diazocin-5(14H)-one (31 mg, 48%) as a white solid. MS ESI calculated for C₂₆H₁₆D₃F₂N₅O₂ [M+H]⁺, 475.17. found 475.05. ¹H NMR (400 MHz, Chloroform-d) δ 8.78 (s, 2H), 8.50 (d, J=7.4 Hz, 1H), 7.73 (d, J=8.2 Hz, 1H), 7.67 (s, 1H), 7.49-7.42 (m, 2H), 7.36 (d, J=8.3 Hz, 1H), 6.86 (t, J=72.7 Hz, 1H), 6.31-6.24 (m, 1H), 5.07-5.00 (m, 1H), 3.57-3.41 (m, 1H), 2.90 (d, J=12.5 Hz, 1H), 2.78 (s, 3H). ¹⁹F NMR (377 MHz, Chloroform-d) δ −80.10, −80.54, −81.01, −81.45.

### Example 22: (7R,14R)-1-(difluoromethoxy)-6-(methyl-d3)-11-(pyrazin-2-ylethynyl)-6,7-dihydro-7,14-methanobenzo[f]benzo[4,5]imidazo[1,2-a][1,4]diazocin-5(14H)-one

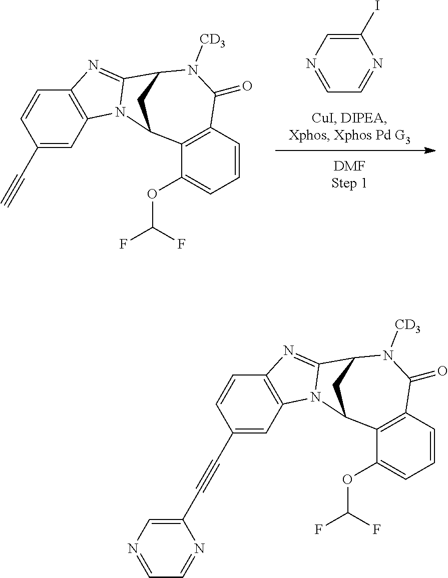

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol) and 2-iodopyrazine (80 mg, 0.393 mmol) in DMF (1 mL) were added CuI (2 mg, 0.013 mmol), Xphos (6 mg, 0.013 mmol), Xphos Pd G₃ (11 mg, 0.013 mmol) and DIPEA (84 mg, 0.655 mmol) at room temperature under nitrogen atmosphere. The resulting mixture was stirred for overnight at 80° C. under nitrogen atmosphere. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-(pyrazin-2-ylethynyl)-6,7-dihydro-7,14-methanobenzo[f]benzo[4,5]imidazo[1,2-a][1,4]diazocin-5(14H)-one (25 mg, 42%) as a white solid. MS ESI calculated for C₂₅H₁₄D₃F₂N₅O₂ [M+H]⁺, 461.15. found 461.10. ¹H NMR (400 MHz, Chloroform-d) δ 8.78 (s, 1H), 8.59 (s, 1H), 8.53-8.46 (m, 2H), 7.76-7.66 (m, 2H), 7.51 (d, J=8.3 Hz, 1H), 7.44 (t, J=8.2 Hz, 1H), 7.37 (d, J=8.0 Hz, 1H), 6.86 (t, J=72.7 Hz, 1H), 6.30-6.21 (m, 1H), 5.06-4.90 (m, 1H), 3.54-3.35 (m, 1H), 2.89 (d, J=13.0 Hz, 1H). ¹⁹F NMR (377 MHz, Chloroform-d) δ −80.02, −80.46, −81.23, −81.67.

### Example 23: (7R,14R)-1-(difluoromethoxy)-6-(methyl-d3)-11-((2-(trifluoromethyl)pyrimidin-5-yl)ethynyl)-6,7-dihydro-7,14-methanobenzo[f]benzo[4,5]imidazo[1,2-a][1,4]diazocin-5(14H)-one

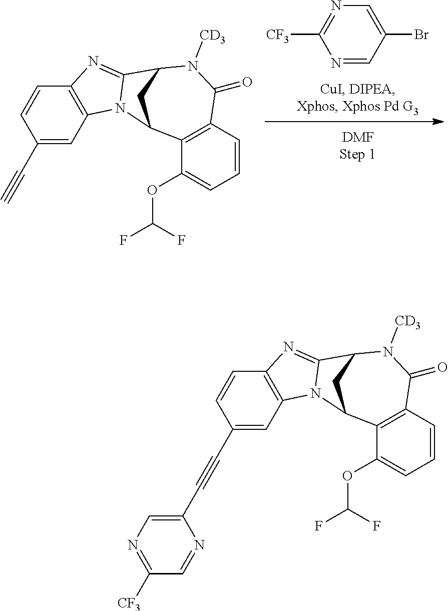

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol) and 5-bromo-2-(trifluoromethyl)pyrimidine (89 mg, 0.393 mmol) in DMF (1 mL) were added CuI (2 mg, 0.013 mmol), Xphos (6 mg, 0.013 mmol), Xphos Pd G₃ (11 mg, 0.013 mmol) and DIPEA (84 mg, 0.655 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((2-(trifluoromethyl)pyrimidin-5-yl)ethynyl)-6,7-dihydro-7,14-methanobenzo[f]benzo[4,5]imidazo[1,2-a][1,4]diazocin-5(14H)-one (34 mg, 49%) as a white solid. MS ESI calculated for C₂₆H₁₃D₃F₅N₅O₂ [M+H]⁺, 529.14. found 529.05. ¹H NMR (400 MHz, Chloroform-d) δ 8.99 (s, 2H), 8.51 (d, J=8.2 Hz, 1H), 7.80-7.67 (m, 2H), 7.52-7.41 (m, 2H), 7.36 (d, J=8.1 Hz, 1H), 6.87 (t, J=72.7 Hz, 1H), 6.29 (d, J=6.9 Hz, 1H), 5.04 (d, J=6.7 Hz, 1H), 3.55-3.45 (m, 1H), 2.91 (d, J=13.6 Hz, 1H). ¹⁹F NMR (377 MHz, Chloroform-d) δ −70.28, −80.18, −80.63, −80.82, −81.27.

### Example 24: 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methyl-1H-pyrazole-5-carbonitrile

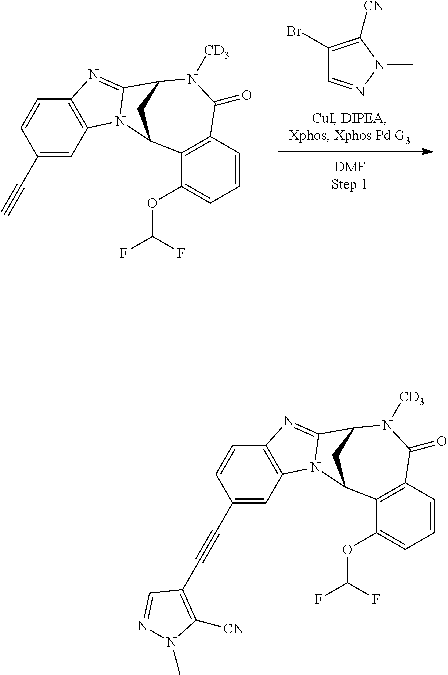

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), DIPEA (115 uL, 0.655 mmol) and 4-bromo-2-methylpyrazole-3-carbonitrile (98 mg, 0.524 mmol) in DMF (1 mL) were added Xphos (7 mg, 0.013 mmol), Xphos Pd G3 (12 mg, 0.013 mmol) and CuI (3 mg, 0.013 mmol) at room temperature under nitrogen atmosphere. The resulting mixture was stirred for overnight at 100° C. under nitrogen atmosphere. The mixture was allowed to cool down to room temperature and diluted with water (20 mL). The mixture was extracted with EtOAc (3×20 mL). The combined organic layers were washed with brine (40 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in Water (10 mmol/L NH₄HCO₃), 35% to 70% gradient in 25 min; detector, 254 nm to afford 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methyl-1H-pyrazole-5-carbonitrile (15 mg, 23%) as a white solid. MS ESI calculated for C₂₆H₁₅D₃F₂N₆O₂[M+H]⁺, 488.16. found 488.05. ¹H NMR (400 MHz, Chloroform-d) δ 8.53-8.44 (m, 1H), 7.72-7.66 (m, 2H), 7.64 (s, 1H), 7.46-7.33 (m, 3H), 6.88 (t, J=73.2 Hz, 1H), 6.25 (d, J=7.2 Hz, 1H), 4.96 (d, J=7.2 Hz, 1H), 4.07 (s, 3H), 3.51-3.40 (m, 1H), 2.88 (d, J=13.6 Hz, 1H). ¹⁹F NMR (377 MHz, Chloroform-d) δ −79.97, −80.42, −81.47, −81.92.

### Example 25: (7R,14R)-1-(difluoromethoxy)-6-(methyl-d3)-11-((4-methyl-4H-1,2,4-triazol-3-yl)ethynyl)-6,7-dihydro-7,14-methanobenzo[f]benzo[4,5]imidazo[1,2-a][1,4]diazocin-5(14H)-one

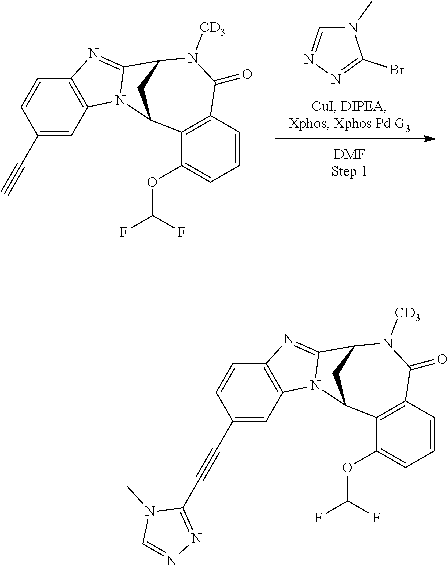

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (63 mg, 0.393 mmol) in DMF (1 mL) were added CuI (2 mg, 0.013 mmol), Xphos (6 mg, 0.013 mmol), Xphos Pd G3 (11 mg, 0.013 mmol) and DIPEA (84 mg, 0.655 mmol) at room temperature under nitrogen atmosphere. The resulting mixture was stirred for 16 h at 80° C. under nitrogen atmosphere. The mixture was allowed to cool down to room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((4-methyl-4H-1,2,4-triazol-3-yl)ethynyl)-6,7-dihydro-7,14-methanobenzo[f]benzo[4,5]imidazo[1,2-a][1,4]diazocin-5(14H)-one (9 mg, 15%) as a white solid. MS ESI calculated for C₂₄H₁₅D₃F₂N₆O₂[M+H]⁺, 464.16. found 464.10. ¹H NMR (400 MHz, Chloroform-d) δ 8.50 (d, J=8.2 Hz, 1H), 8.20 (s, 1H), 7.77-7.69 (m, 2H), 7.47-7.42 (m, 2H), 7.36 (d, J=8.0 Hz, 1H), 6.88 (t, J=72.7 Hz, 1H), 6.26 (d, J=7.1 Hz, 1H), 4.99 (d, J=6.8 Hz, 1H), 3.81 (s, 3H), 3.53-3.45 (m, 1H), 2.90 (d, J=13.6 Hz, 1H). ¹⁹F NMR (377 MHz, Chloroform-d) δ −80.31, −80.75, −81.12, −81.57.

### Example 26: (7R,14R)-1-(difluoromethoxy)-6-(methyl-d3)-11-((6-methylpyridazin-3-yl)ethynyl)-6,7-dihydro-7,14-methanobenzo[f]benzo[4,5]imidazo[1,2-a][1,4]diazocin-5(14H)-one

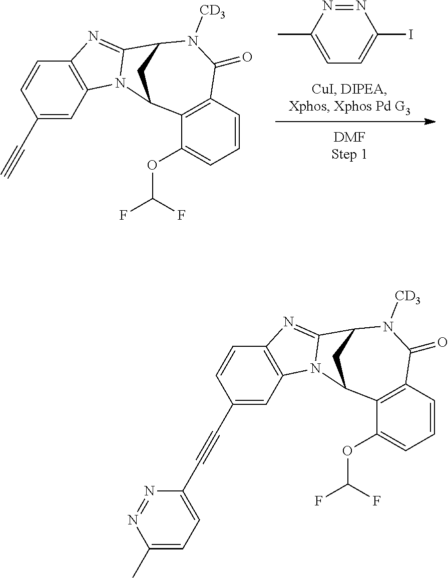

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.078 mmol) and 3-iodo-6-methylpyridazine (143 mg, 0.655 mmol) in DMF (1 mL) were added CuI (2 mg, 0.013 mmol), Xphos (6 mg, 0.013 mmol), Xphos Pd G₃ (11 mg, 0.013 mmol) and DIPEA (84 mg, 0.655 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((6-methylpyridazin-3-yl)ethynyl)-6,7-dihydro-7,14-methanobenzo[f]benzo[4,5]imidazo[1,2-a][1,4]diazocin-5(14H)-one (40 mg, 63%) as a white solid. MS ESI calculated for C₂₆H₁₆D₃F₂N₅O₂ [M+H]⁺, 475.17. found 475.10. ¹H NMR (400 MHz, Chloroform-d) δ 8.50 (d, J=8.1 Hz, 1H), 7.77-7.68 (m, 2H), 7.56-7.49 (m, 2H), 7.44 (t, J=8.1 Hz, 1H), 7.38 (d, J=8.0 Hz, 1H), 7.33 (d, J=8.6 Hz, 1H), 6.88 (t, J=72.6 Hz, 1H), 6.26 (d, J=7.2 Hz, 1H), 5.00 (d, J=7.1 Hz, 1H), 3.52-3.43 (m, 1H), 2.89 (d, J=13.5 Hz, 1H), 2.77 (s, 3H). ¹⁹F NMR (377 MHz, Chloroform-d) δ −80.04, −80.48, −81.38, −81.83.

### Example 27: methyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate

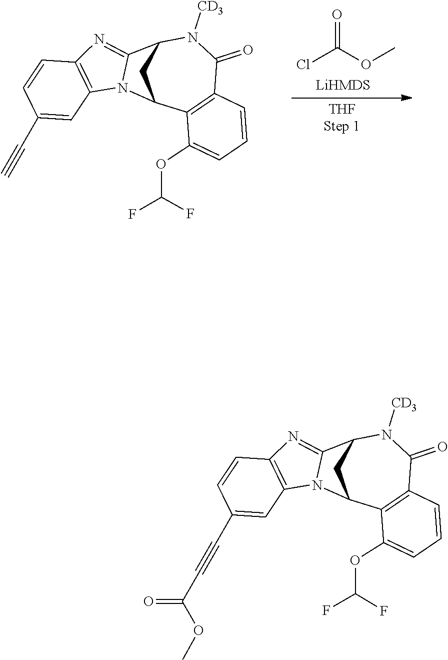

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol) in THF (2 mL) was added LiHMDS (0.20 mL, 0.197 mmol, 1M in THF) dropwise at −78° C. under nitrogen atmosphere. The resulting mixture was stirred for 30 min at −78° C. under nitrogen atmosphere. To the above mixture was added methyl chloroformate (25 mg, 0.262 mmol) dropwise portions at −78° C. The resulting mixture was stirred for additional overnight at room temperature. The reaction was quenched with 10 mL sat. NH₄Cl (aq.) at room temperature and the mixture was extracted with EtOAc (3×10 mL). The combined organic layers were washed with brine (20 mL), dried over anhydrous Na₂SO₄. After filtration, the filtrate was concentrated under reduced pressure. The crude product (70 mg) was purified by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min mL/min; Gradient: isocratic 27% B to 47% B in 10 min; Wave Length: 254 nm/220 nm nm) to afford methyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate (16 mg, 26%) as a white solid. MS ESI calculated for C₂₃H₁₄D₃F₂N₃O₄[M+H]⁺, 441.14. found 441.10. ¹H NMR (400 MHz, Chloroform-d) δ 8.52-8.44 (m, 1H), 7.79-7.59 (m, 2H), 7.48-7.39 (m, 2H), 7.37 (d, J=8.0 Hz, 1H), 6.85 (t, J=72.8 Hz, 1H), 6.23 (d, J=7.2 Hz, 1H), 4.95 (d, J=7.2 Hz, 1H), 3.85 (s, 3H), 3.52-3.38 (m, 1H), 2.87 (d, J=13.6 Hz, 1H). ¹⁹F NMR (377 MHz, Chloroform-d) δ −80.06, −80.50, −81.36, −81.80.

### Example 28: (7R,14R)-1-(difluoromethoxy)-6-(methyl-d3)-11-((5-methyl-1,3,4-oxadiazol-2-yl)ethynyl)-6,7-dihydro-7,14-methanobenzo[f]benzo[4,5]imidazo[1,2-a][1,4]diazocin-5(14H)-one

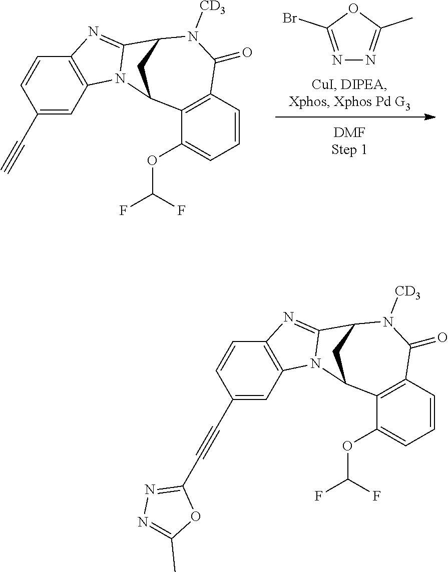

Into a 8 mL vial were added (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), 2-bromo-5-methyl-1,3,4-oxadiazole (85 mg, 0.524 mmol), CuI (2 mg, 0.013 mmol), Xphos (13 mg, 0.026 mmol), Xphos Pd G3 (22 mg, 0.026 mmol), DIPEA (115 uL, 0.655 mmol) and DMF (0.5 mL) at room temperature. The mixture was purged with nitrogen for 5 min and then was stirred for 5 h at 90° C. The mixture was allowed to cool down to room temperature. The resulting mixture was diluted with water (15 mL) and ethyl acetate (10 mL). The mixture was extracted with ethyl acetate (2×10 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in Water (10 mmol/L NH₄HCO₃), 35% to 70% gradient in 25 min; detector, 254 nm to afford (7R,14R)-1-(difluoromethoxy)-6-(methyl-d3)-11-((5-methyl-1,3,4-oxadiazol-2-yl)ethynyl)-6,7-dihydro-7,14-methanobenzo[f]benzo[4,5]imidazo[1,2-a][1,4]diazocin-5(14H)-one (14 mg, 21%) as a white solid. MS ESI calculated for C₂₄H₁₄D₃F₂N₅O₃ [M+H]⁺, 465.15. found 465.10. ¹H NMR (400 MHz, Chloroform-d) δ 8.50 (d, J=8.0 Hz, 1H), 7.85-7.66 (m, 2H), 7.48 (d, J=10.0 Hz, 1H), 7.44 (d, J=8.0 Hz, 1H), 7.38 (d, J=8.0 Hz, 1H), 6.87 (t, J=72.8 Hz, 1H), 6.27 (d, J=6.0 Hz, 1H), 5.06-4.91 (m, 1H), 3.53-3.40 (m, 1H), 2.90 (d, J=12.8 Hz, 1H), 2.60 (s, 3H). ¹⁹F NMR (377 MHz, Chloroform-d) δ −80.10, −80.54, −81.25, −81.69.

### Example 29: (7R,14R)-1-(difluoromethoxy)-6-(methyl-d3)-11-((5-methyl-1,3,4-thiadiazol-2-yl)ethynyl)-6,7-dihydro-7,14-methanobenzo[f]benzo[4,5]imidazo[1,2-a][1,4]diazocin-5(14H)-one

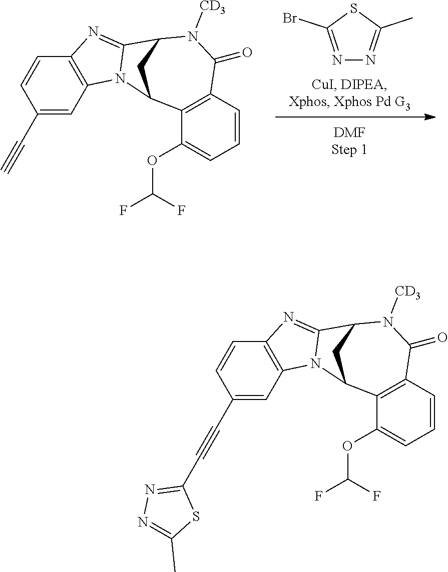

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), XPhos (12 mg, 0.026 mmol), XPhos Pd G₃ (22 mg, 0.026 mmol) and 2-bromo-5-methyl-1,3,4-thiadiazole (93 mg, 0.524 mmol) in DMF (1 mL) were added DIPEA (113 uL, 0.655 mmol) and CuI (2 mg, 0.013 mmol). The resulting mixture was stirred for overnight at 100° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((5-methyl-1,3,4-thiadiazol-2-yl)ethynyl)-6,7-dihydro-7,14-methanobenzo[f]benzo[4,5]imidazo[1,2-a][1,4]diazocin-5(14H)-one (21 mg, 32%) as a white solid. MS ESI calculated for C₂₄H₁₄D₃F₂N₅O₂S [M+H]⁺, 481.13. found 481.00. ¹H NMR (400 MHz, Chloroform-d) δ 8.52-8.48 (m, 1H), 7.74 (d, J=9.0 Hz, 2H), 7.52-7.48 (m, 1H), 7.45 (t, J=8.2 Hz, 1H), 7.39 (d, J=8.1 Hz, 1H), 7.08-6.67 (m, 1H), 6.28 (d, J=7.1 Hz, 1H), 5.06 (d, J=6.9 Hz, 1H), 3.54-3.47 (m, 1H), 2.91 (d, J=13.6 Hz, 1H), 2.83 (s, 3H). ¹⁹F NMR (377 MHz, Chloroform-d) δ −80.14, −80.58, −81.35, −81.80.

### Example 30: (7R,14R)-11-((1,3,4-thiadiazol-2-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

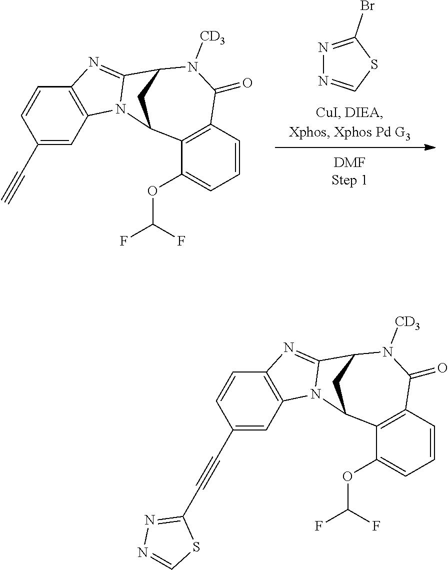

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), XPhos (12 mg, 0.026 mmol), XPhos Pd G₃ (22 mg, 0.026 mmol) and 2-bromo-1,3,4-thiadiazole (86 mg, 0.524 mmol) in DMF (0.5 mL) were added DIPEA (113 uL, 0.655 mmol) and CuI (2 mg, 0.013 mmol). The resulting mixture was stirred for overnight at 100° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-11-((1,3,4-thiadiazol-2-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (9 mg, 15%) as a white solid. MS ESI calculated for C₂₃H₁₂D₃F₂N₅O₂S [M+H]⁺, 467.11. found 467.05. ¹H NMR (400 MHz, Chloroform-d) δ 9.17 (s, 1H), 8.51 (d, J=8.1 Hz, 1H), 7.79 (d, J=8.5 Hz, 2H), 7.56 (d, J=8.8 Hz, 1H), 7.48 (t, J=8.2 Hz, 1H), 7.40 (d, J=8.1 Hz, 1H), 6.88 (t, J=72.4 Hz, 1H), 6.33 (d, J=6.4 Hz, 1H), 5.18-5.15 (m, 1H), 3.63-3.46 (m, 1H), 2.94 (d, J=13.3 Hz, 1H). ¹⁹F NMR (377 MHz, Chloroform-d) δ −80.23, −80.67, −81.29, −81.73.

### Example 31: (7R,14R)-1-(difluoromethoxy)-11-((5-fluoropyridin-3-yl)ethynyl)-6-(methyl-d3)-6,7-dihydro-7,14-methanobenzo[f]benzo[4,5]imidazo[1,2-a][1,4]diazocin-5(14H)-one

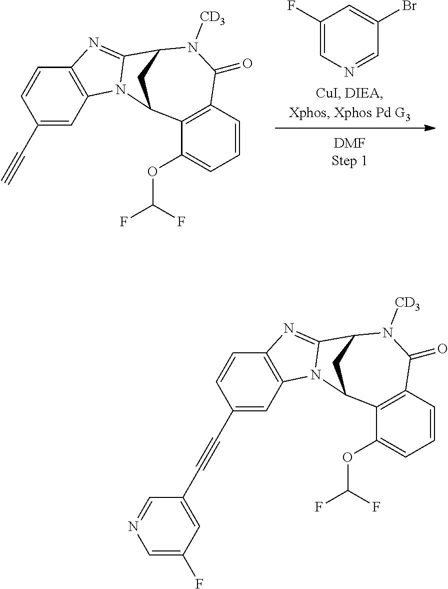

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (40 mg, 0.105 mmol) and 3-bromo-5-fluoropyridine (55 mg, 0.315 mmol) in DMF (1 mL) were added CuI (1 mg, 0.011 mmol), XPhos (4 mg, 0.011 mmol), XPhos Pd G₃ (4 mg, 0.011 mmol) and DIPEA (67 mg, 0.525 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-((5-fluoropyridin-3-yl)ethynyl)-6-(methyl-d3)-6,7-dihydro-7,14-methanobenzo[f]benzo[4,5]imidazo[1,2-a][1,4]diazocin-5(14H)-one (30 mg, 57%) as a white solid. MS ESI calculated for C₂₆H₁₄D₃F₃N₄O₂ [M+H]⁺, 478.15. found 478.05. ¹H NMR (400 MHz, Chloroform-d) δ 8.60 (s, 1H), 8.51 (d, J=8.3 Hz, 1H), 8.44 (s, 1H), 7.74 (d, J=8.6 Hz, 1H), 7.69 (s, 1H), 7.54 (d, J=8.7 Hz, 1H), 7.46 (t, J=8.1 Hz, 2H), 7.37 (d, J=8.1 Hz, 1H), 6.87 (t, J=72.6 Hz, 1H), 6.30 (d, J=6.8 Hz, 1H), 5.09 (d, J=6.1 Hz, 1H), 3.58-3.43 (m, 1H), 2.92 (d, J=13.5 Hz, 1H). ¹⁹F NMR (377 MHz, Chloroform-d)) δ −80.14, −80.58, −81.01, −81.46, −126.61.

### Example 32: (7R,14R)-1-(difluoromethoxy)-6-(methyl-d3)-11-(thiazol-5-ylethynyl)-6,7-dihydro-7,14-methanobenzo[f]benzo[4,5]imidazo[1,2-a][1,4]diazocin-5(14H)-one

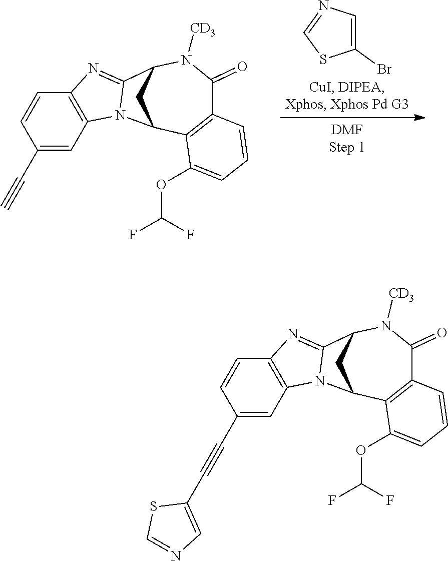 [SMILES: [2H]C([2H])([2H])N1C(=O)c2cccc(OC(F)F)c2[C@H]2C[C@@H]1c1nc3ccc(C#C)cc3n12 ; Brc1cncs1 ; [2H]C([2H])([2H])N1C(=O)c2cccc(OC(F)F)c2[C@H]2C[C@@H]1c1nc3ccc(C#Cc4cncs4)cc3n12]

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (40 mg, 0.105 mmol) and 5-bromo-1,3-thiazole (51 mg, 0.315 mmol) in DMF (1 mL) were added CuI (1 mg, 0.011 mmol), XPhos (4 mg, 0.011 mmol), XPhos Pd G₃ (8 mg, 0.011 mmol) and DIPEA (67 mg, 0.525 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-(thiazol-5-ylethynyl)-6,7-dihydro-7,14-methanobenzo[f]benzo[4,5]imidazo[1,2-a][1,4]diazocin-5(14H)-one (18 mg, 38%) as a white solid. MS ESI calculated for C₂₄H₁₃D₃F₂N₄O₂S [M+H]⁺, 466.12. found 466.05. ¹H NMR (400 MHz, Chloroform-d) δ 8.76 (s, 1H), 8.50 (d, J=8.2 Hz, 1H), 8.07 (s, 1H), 7.72 (d, J=8.5 Hz, 1H), 7.64 (s, 1H), 7.49-7.39 (m, 2H), 7.37 (d, J=8.0 Hz, 1H), 6.86 (t, J=72.7 Hz, 1H), 6.27 (d, J=7.2 Hz, 1H), 5.04 (d, J=6.9 Hz, 1H), 3.53-3.45 (m, 1H), 2.90 (d, J=13.7 Hz, 1H). ¹⁹F NMR (377 MHz, Chloroform-d)) δ −80.03, −80.47, −81.12, −81.56.

### Example 33: (7R,14R)-1-(difluoromethoxy)-6-(methyl-d3)-11-(pyridazin-3-ylethynyl)-6,7-dihydro-7,14-methanobenzo[f]benzo[4,5]imidazo[1,2-a][1,4]diazocin-5(14H)-one

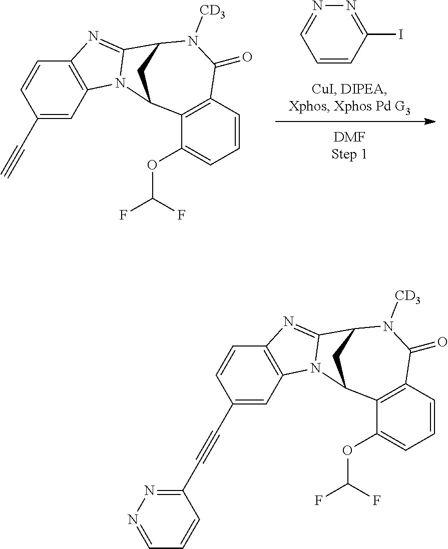

Into a 8 mL vial were added (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), 3-iodopyridazine (81 mg, 0.393 mmol), DIPEA (114 uL, 0.655 mmol), XPhos (6 mg, 0.013 mmol), XPhos Pd G3 (11 mg, 0.013 mmol), CuI (1 mg, 0.007 mmol) and DMF (1 mL) at room temperature. The mixture was purged with nitrogen for 5 min and then was stirred for 5 h at 90° C. The mixture was allowed to cool down to room temperature. The resulting mixture was diluted with water (15 mL) and ethyl acetate (10 mL). The mixture was extracted with ethyl acetate (2×10 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in Water (10 mmol/L NH₄HCO₃), 35% to 70% gradient in 25 min; detector, 254 nm to afford (7R,14R)-1-(difluoromethoxy)-6-(methyl-d3)-11-(pyridazin-3-ylethynyl)-6,7-dihydro-7,14-methanobenzo[f]benzo[4,5]imidazo[1,2-a][1,4]diazocin-5(14H)-one (24 mg, 38%) as a white solid. MS ESI calculated for C₂₅H₁₄D₃F₂N₅O₂ [M+H]⁺, 461.15. found 461.00. ¹H NMR (400 MHz, Chloroform-d) δ 9.18-9.10 (m, 1H), 8.54-8.46 (m, 1H), 7.75 (d, J=1.6 Hz, 1H), 7.72 (d, J=8.4 Hz, 1H), 7.669-7.64 (m, 1H), 7.54-7.41 (m, 3H), 7.38 (d, J=8.0 Hz, 1H), 6.88 (t, J=72.8 Hz, 1H), 6.27 (d, J=6.8 Hz, 1H), 5.00 (d, J=6.4 Hz, 1H), 3.61-3.41 (m, 1H), 2.89 (d, J=13.6 Hz, 1H). ¹⁹F NMR (376 MHz, Chloroform-d) δ −80.06, −80.51, −81.33, −81.77.

### Example 34: (7R,14R)-1-(difluoromethoxy)-6-(methyl-d3)-11-((5-methylpyrazin-2-yl)ethynyl)-6,7-dihydro-7,14-methanobenzo[f]benzo[4,5]imidazo[1,2-a][1,4]diazocin-5(14H)-one

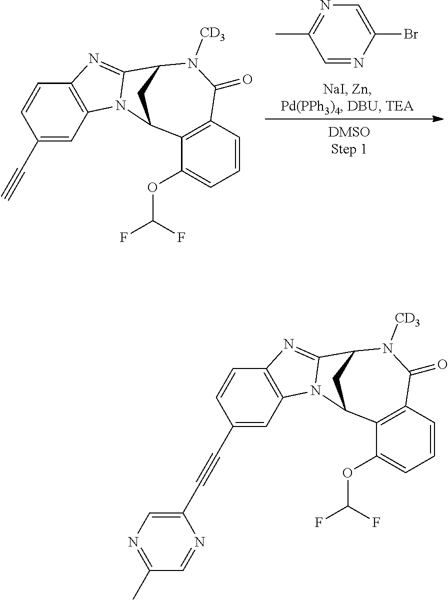

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (226 mg, 1.310 mmol) and 2-bromo-5-methylpyrazine (102 mg, 1.310 mmol) in DMSO (1 mL) were added NaI (3 mg, 0.026 mmol), Zn (1 mg, 0.026 mmol), Pd(PPh₃)₄(30 mg, 0.026 mmol), DBU (39 mg, 0.262 mmol) and TEA (39 mg, 0.262 mmol) at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-6-(methyl-d3)-11-((5-methylpyrazin-2-yl)ethynyl)-6,7-dihydro-7,14-methanobenzo[f]benzo[4,5]imidazo[1,2-a][1,4]diazocin-5(14H)-one (4 mg, 7%) as a white solid. MS ESI calculated for C₂₆H₁₆D₃F₂N₅O₂ [M+H]⁺, 475.17. found 475.05. ¹H NMR (400 MHz, Chloroform-d) δ 8.66 (d, J=1.3 Hz, 1H), 8.51-8.48 (m, 1H), 8.46 (s, 1H), 7.76-7.66 (m, 2H), 7.50-7.48 (m, 1H), 7.43 (t, J=8.2 Hz, 1H), 7.37 (d, J=8.1 Hz, 1H), 7.05-6.67 (m, 1H), 6.25 (d, J=7.3 Hz, 1H), 4.98 (d, J=7.0 Hz, 1H), 3.50-3.43 (m, 1H), 2.88 (d, J=13.6 Hz, 1H), 2.61 (s, 3H). ¹⁹F NMR (377 MHz, Chloroform-d) δ −80.00, −80.44, −81.30, −81.74.

### Example 35: (7R,14R)-1-(difluoromethoxy)-11-((1-hydroxycyclobutyl)ethynyl)-6-(methyl-d3)-6,7-dihydro-7,14-methanobenzo[f]benzo[4,5]imidazo[1,2-a][1,4]diazocin-5(14H)-one

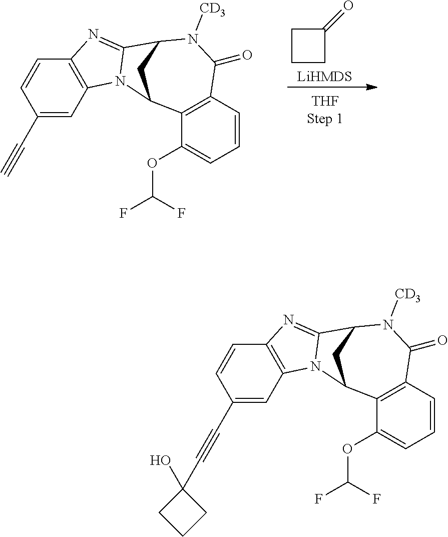

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol) in THF (2 mL) was added LiHMDS (0.20 mL, 0.197 mmol, 1 M in THF) dropwise at −78° C. under nitrogen atmosphere. The resulting mixture was stirred for 30 min at −78° C. under nitrogen atmosphere. To the above mixture was added a solution of cyclobutanone (19 mg, 0.262 mmol) in THF (0.5 mL) dropwise over 5 min at −78° C. The resulting mixture was stirred for additional 3 h at room temperature. The resulting mixture was diluted with water (15 mL) and extracted with EtOAc (3×15 mL). The combined organic layers were washed with brine (30 mL), dried over anhydrous Na₂SO₄. After filtration, the filtrate was concentrated under reduced pressure. The crude product (70 mg) was purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min; Gradient: 27.5% B to 45% B in 10 min; Wave Length: 254 nm/220 nm nm) to afford (7R,14R)-1-(difluoromethoxy)-11-((1-hydroxycyclobutyl)ethynyl)-6-(methyl-d3)-6,7-dihydro-7,14-methanobenzo[f]benzo[4,5]imidazo[1,2-a][1,4]diazocin-5(14H)-one (24 mg, 41%) as a white solid. MS ESI calculated for C₂₅H₁₈D₃F₂N₃O₃ [M+H]⁺, 453.17. found 453.10. ¹H NMR (400 MHz, Chloroform-d) δ 8.51-8.44 (m, 1H), 7.63 (d, J=8.4 Hz, 1H), 7.54 (s, 1H), 7.42 (t, J=8.0 Hz, 1H), 7.34 (d, J=8.0 Hz, 1H), 7.30 (d, J=8.4 Hz, 1H), 6.85 (t, J=72.8 Hz, 1H), 6.27-6.17 (m, 1H), 5.06-4.88 (m, 1H), 3.53-3.37 (m, 1H), 2.87 (d, J=10.0 Hz, 1H), 2.61-2.48 (m, 2H), 2.42-2.30 (m, 2H), 1.93-1.82 (m, 2H). ¹⁹F NMR (377 MHz, Chloroform-d) δ −80.01, −80.46, −81.26, −81.70.

### Example 36: (7R,14R)-1-(difluoromethoxy)-11-(isothiazol-4-ylethynyl)-6-(methyl-d3)-6,7-dihydro-7,14-methanobenzo[f]benzo[4,5]imidazo[1,2-a][1,4]diazocin-5(14H)-one

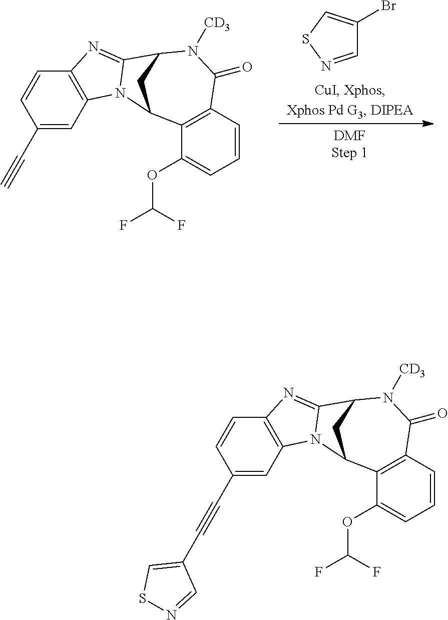

A solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), 4-bromo-1,2-thiazole (64 mg, 0.393 mmol), CuI (2 mg, 0.013 mmol), XPhos (6 mg, 0.013 mmol), XPhos Pd G3 (11 mg, 0.013 mmol) and DIPEA (84 mg, 0.655 mmol) in DMF (1 mL) was stirred for 16 h at 80° C. under nitrogen atmosphere. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-(isothiazol-4-ylethynyl)-6-(methyl-d3)-6,7-dihydro-7,14-methanobenzo[f]benzo[4,5]imidazo[1,2-a][1,4]diazocin-5(14H)-one (19 mg, 30%) as a white solid. MS ESI calculated for C₂₄H₁₃D₃F₂N₄O₂S [M+H]⁺, 466.12. found 466.05. ¹H NMR (400 MHz, Chloroform-d) δ 8.77 (s, 1H), 8.61 (s, 1H), 8.51 (d, J=8.2 Hz, 1H), 7.73 (d, J=8.5 Hz, 1H), 7.66 (s, 1H), 7.49-7.43 (m, 2H), 7.37 (d, J=8.1 Hz, 1H), 6.86 (t, J=72.6 Hz, 1H), 6.29 (d, J=7.2 Hz, 1H), 5.11 (d, J=7.1 Hz, 1H), 3.56-3.47 (m, 1H), 2.92 (d, J=13.6 Hz, 1H). ¹⁹F NMR (377 MHz, Chloroform-d)) δ −80.07, −80.52, −80.99, −81.43.

### Example 37 and 38: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic acid and isopropyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate

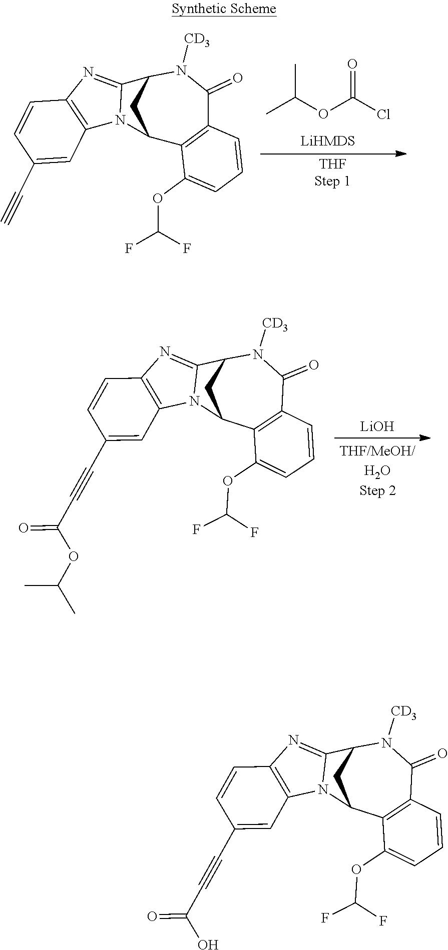

### Example 38: isopropyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate

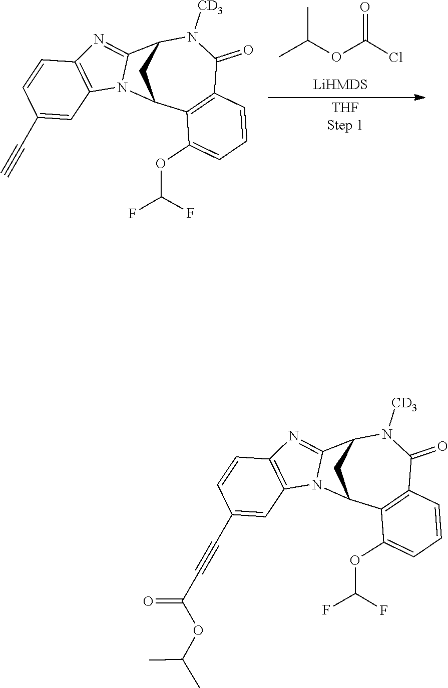

A mixture of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (190 mg, 0.497 mmol) and LiHMDS (0.6 mL, 0.596 mmol, 1 M) in THF (4 mL) was stirred for 1 h at −78° C. under nitrogen atmosphere. To the above mixture was added isopropyl chloroformate (110 mg, 0.895 mmol) dropwise at −78° C. The resulting mixture was stirred for additional 16 h at room temperature. The reaction was quenched with water (1 drop) at room temperature. The resulting mixture was concentrated under reduced pressure. The residue was purified by reversed-phase flash chromatography with the following conditions: column, C18 silica gel; mobile phase, CH₃CN in water (10 mmol/L NH₄HCO₃), 30% to 60% gradient in 25 min; detector, 254 nm. This resulted in isopropyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate (105 mg, 44%) as a white solid. MS ESI calculated for C₂₅H₁₈D₃F₂N₃O₄ [M+H]⁺, 469.17. found 469.10. ¹H NMR (400 MHz, DMSO-d₆) δ 8.29-8.26 (m, 1H), 7.79-7.56 (m, 3H), 7.55-7.39 (m, 3H), 6.28 (d, J=7.1 Hz, 1H), 5.27 (d, J=7.1 Hz, 1H), 5.09-5.02 (m, 1H), 3.54-3.48 (m, 1H), 2.83 (d, J=13.8 Hz, 1H), 1.28 (d, J=6.2, 6H). ¹⁹F NMR (377 MHz, DMSO-d₆) δ −81.56, −82.01, −82.17, −82.61.

### Example 37: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic Acid

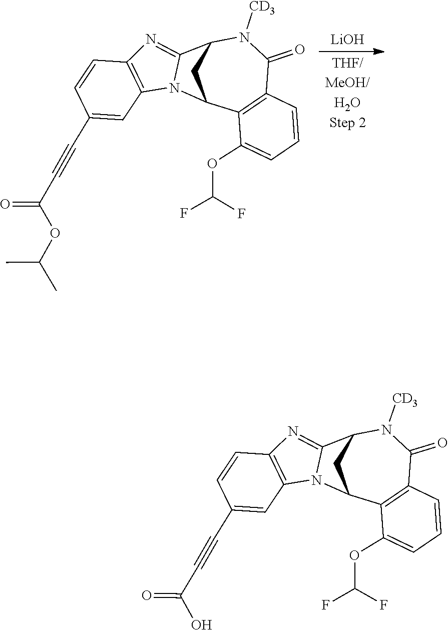

A mixture of isopropyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate (85 mg, 0.181 mmol) and LiOH (38 mg, 0.905 mmol) in THF (0.2 mL), MeOH (0.4 mL) and H₂O (0.4 mL) was stirred for 16 h at room temperature. The residue was purified by reversed-phase flash chromatography with the following conditions: column, C18 silica gel; mobile phase, CH₃CN in water (0.1% FA), 15% to 45% gradient in 30 min; detector, 254 nm. This resulted in 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic acid (55 mg, 70%) as a white solid. MS ESI calculated for C₂₂H₁₂D₃F₂N₃O₄ [M+H]⁺, 427.12. found 427.05. ¹H NMR (400 MHz, DMSO-d₆) δ 12.40 (s, 1H), 8.30-8.26 (m, 1H), 7.80-7.58 (m, 3H), 7.56-7.48 (m, 2H), 7.44-7.40 (m, 1H), 6.28 (d, J=7.1 Hz, 1H), 5.26 (d, J=7.1 Hz, 1H), 3.55-3.48 (m, 1H), 2.83 (d, J=13.8 Hz, 1H). ¹⁹F NMR (377 MHz, DMSO-d₆) δ −81.56, −82.00, −82.10, −82.54.

### Example 39: (7R,14R)-1-(difluoromethoxy)-6-(methyl-d3)-11-(prop-1-yn-1-yl)-6,7-dihydro-7,14-methanobenzo[f]benzo[4,5]imidazo[1,2-a][1,4]diazocin-5(14H)-one

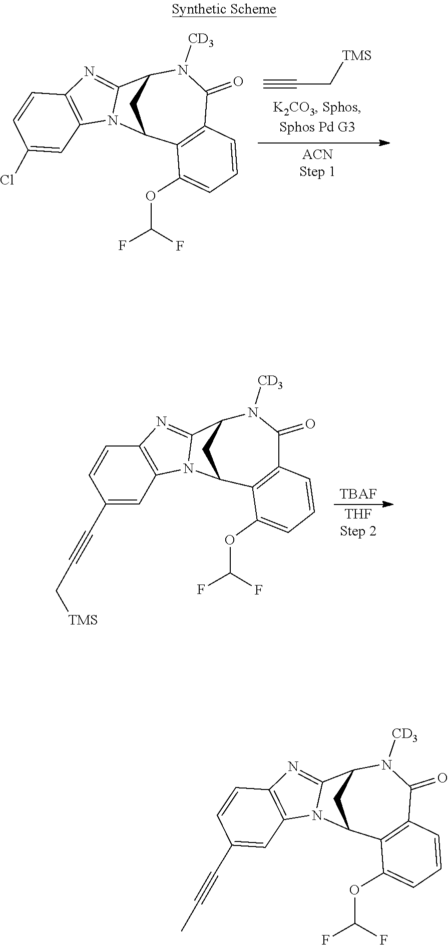

### Preparation 39A: (7R,14R)-1-(difluoromethoxy)-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one

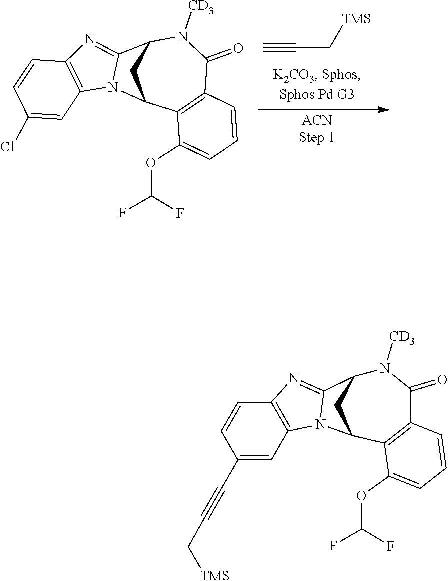

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (50 mg, 0.127 mmol), trimethyl(prop-2-yn-1-yl)silane (71 mg, 0.635 mmol), K₂CO₃ (88 mg, 0.635 mmol), SPhos Pd Gen.3 (20 mg, 0.025 mmol), SPhos (10 mg, 0.025 mmol) and ACN (1 mL) at room temperature. The mixture was purged with nitrogen for 5 min and then was stirred for overnight at 80° C. under nitrogen atmosphere. The resulting mixture was concentrated under vacuum. The residue was purified by silica gel column chromatography, eluted with CH₂Cl₂/MeOH (12/1) to afford (7R,14R)-1-(difluoromethoxy)-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one (70 mg) as a yellow oil. The crude product was used in the next step directly without further purification. MS ESI calculated for C₂₅H₂₂D₃F₂N₃O₂Si [M+H]⁺, 469.19. found 469.10.

### Example 39: (7R,14R)-1-(difluoromethoxy)-6-(methyl-d3)-11-(prop-1-yn-1-yl)-6,7-dihydro-7,14-methanobenzo[f]benzo[4,5]imidazo[1,2-a][1,4]diazocin-5(14H)-one

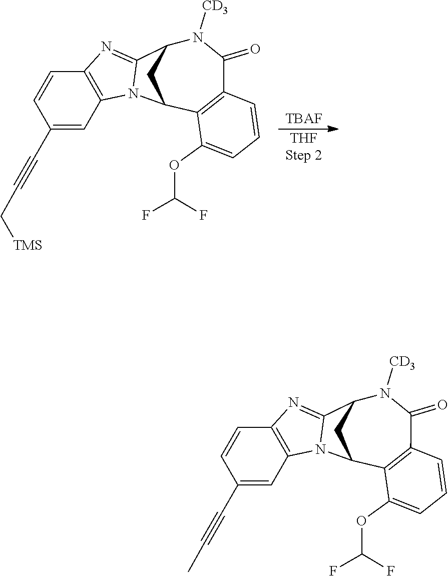

To a stirred solution of (7R,14R)-1-(difluoromethoxy)-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one (70 mg, 0.149 mmol) in THF (3 mL) was added TBAF (179 uL, 0.179 mmol, 1M in THF) dropwise at room temperature. The resulting mixture was stirred for 1 h at room temperature and then concentrated under vacuum. The residue was purified by silica gel column chromatography, eluted with CH₂Cl₂/MeOH (12/1) followed by Prep-HPLC with the following conditions (Column: Xselect CSH F-Phenyl OBD column 30*250 mm, 5 μm; Mobile Phase A: Water (10 mmol/L NH₄HCO₃+0.1% NH₃·H₂O), Mobile Phase B: MeOH; Flow rate: 60 mL/min mL/min; Gradient: 47% B to 62% B in 10 min; Wave Length: 254 nm/220 nm nm) to afford (7R,14R)-1-(difluoromethoxy)-6-(methyl-d3)-11-(prop-1-yn-1-yl)-6,7-dihydro-7,14-methanobenzo[f]benzo[4,5]imidazo[1,2-a][1,4]diazocin-5(14H)-one (9 mg, 15%) as a white solid. MS ESI calculated for C₂₂H₁₄D₃F₂N₃O₂ [M+H]⁺, 397.15. found 397.10. ¹H NMR (400 MHz, Chloroform-d) δ 8.50-8.43 (m, 1H), 7.60 (d, J=8.4 Hz, 1H), 7.48 (d, J=1.6 Hz, 1H), 7.40 (t, J=8.4 Hz, 1H), 7.33 (d, J=8.0 Hz, 1H), 7.28-7.24 (m, 1H), 6.83 (t, J=73.2 Hz, 1H), 6.18 (d, J=7.2 Hz, 1H), 4.91 (d, J=7.2 Hz, 1H), 3.47-3.34 (m, 1H), 2.83 (d, J=13.6 Hz, 1H), 2.06 (s, 3H). ¹⁹F NMR (377 MHz, Chloroform-d) δ −79.79, −80.23, −81.09, −81.53.

### Example 40: ethyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate

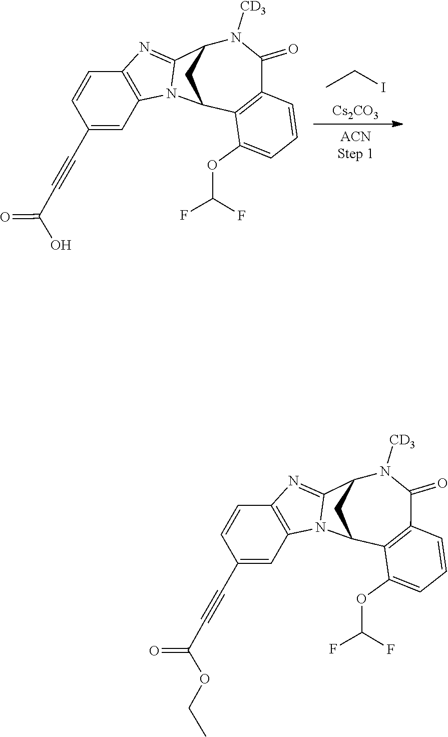

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic acid (27 mg, 0.063 mmol) and ethyl iodide (39 mg, 0.252 mmol) in ACN (1 mL) was added Cs₂CO₃ (41 mg, 0.126 mmol) at room temperature. The resulting mixture was stirred for 16 h at 50° C. The resulting mixture was concentrated under reduced pressure. The residue was purified by reversed-phase flash chromatography with the following conditions: column, C18 silica gel; mobile phase, CH₃CN in water (10 mmol/L NH₄HCO₃), 35% to 65% gradient in 25 min; detector, 254 nm. This resulted in ethyl 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolate (20 mg, 71%) as a white solid. MS ESI calculated for C₂₄H₁₆D₃F₂N₃O₄ [M+H]⁺, 455.15. found 455.15. ¹H NMR (400 MHz, DMSO-d₆) δ 8.30-8.26 (m, 1H), 7.80-7.54 (m, 3H), 7.53-7.41 (m, 3H), 6.28 (d, J=7.1 Hz, 1H), 5.27 (d, J=7.1 Hz, 1H), 4.25 (q, J=7.1 Hz, 2H), 3.56-3.47 (m, 1H), 2.84 (d, J=13.8 Hz, 1H), 1.27 (t, J=7.1 Hz, 3H). ¹⁹F NMR (377 MHz, DMSO-d₆) δ −81.58, −82.03, −82.18, −82.62.

### Example 41: (7R,14R)-1-(difluoromethoxy)-11-(isoxazol-4-ylethynyl)-6-(methyl-d3)-6,7-dihydro-7,14-methanobenzo[f]benzo[4,5]imidazo[1,2-a][1,4]diazocin-5(14H)-one

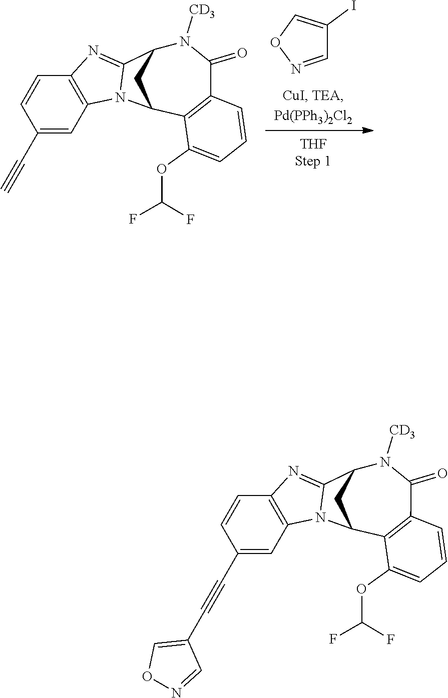

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (100 mg, 0.251 mmol), TEA (105 uL, 0.753 mmol) and 4-iodo-1,2-oxazole (147 mg, 0.753 mmol) in THF (1 mL) were added Pd(PPh₃)2C₂ (18 mg, 0.025 mmol) and CuI (4 mg, 0.025 mmol). After stirring for 1 h at room temperature under a nitrogen atmosphere, the mixture was diluted with water (10 mL) and extracted with EtOAc (3×10 mL). The combined organic layers were washed with brine (20 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in water (10 mmol/L NH₄HCO₃), 35% to 70% gradient in 25 min; detector, 254 nm to afford (7R,14R)-1-(difluoromethoxy)-11-(isoxazol-4-ylethynyl)-6-(methyl-d3)-6,7-dihydro-7,14-methanobenzo[f]benzo[4,5]imidazo[1,2-a][1,4]diazocin-5(14H)-one (6 mg, 5%) as a white solid. MS ESI calculated for C₂₄H₁₃D₃F₂N₄O₃ [M+H]⁺, 450.14. found 450.10. ¹H NMR (400 MHz, Chloroform-d) δ 8.65 (s, 1H), 8.53-5.46 (m, 1H), 8.40 (s, 1H), 7.69 (d, J=8.4 Hz, 1H), 7.61 (s, 1H), 7.47-7.32 (m, 3H), 6.85 (t, J=72.8 Hz, 1H), 6.25 (d, J=6.8 Hz, 1H), 5.04-4.95 (m, 1H), 3.54-3.39 (m, 1H), 2.88 (d, J=13.6 Hz, 1H). ¹⁹F NMR (377 MHz, Chloroform-d) δ −79.99, −80.44, −80.88, −81.33.

### Example 42: (7R,14R)-1-(difluoromethoxy)-11-(3-methoxyprop-1-yn-1-yl)-6-(methyl-d3)-6,7-dihydro-7,14-methanobenzo[f]benzo[4,5]imidazo[1,2-a][1,4]diazocin-5(14H)-one

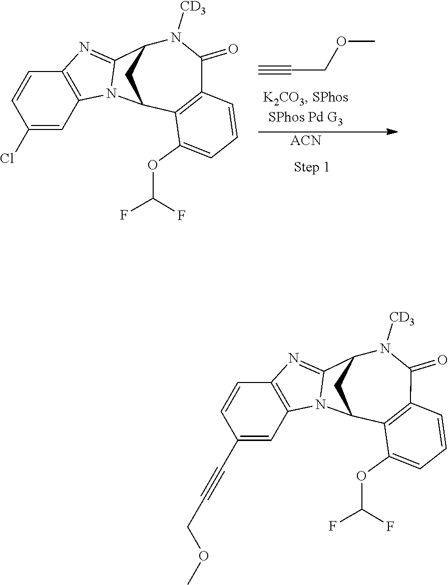

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (50 mg, 0.127 mmol), K₂CO₃ (88 mg, 0.635 mmol), SPhos (10 mg, 0.025 mmol), SPhos Pd Gen.3 (19 mg, 0.025 mmol), 3-methoxy-propyne (44 mg, 0.635 mmol) and ACN (2 mL) at room temperature. The mixture was purged with nitrogen for 3 min and then was stirred overnight at 80° C. under nitrogen atmosphere. The mixture was concentrated under reduce pressure. The residue was purified by silica gel column chromatography, eluted with CH₂Cl₂/MeOH (12/1) followed by Prep-HPLC with the following conditions (Column: Xselect CSH F-Phenyl OBD column 30*250 mm, 5 μm; Mobile Phase A: Water (0.1% FA), Mobile Phase B: MeOH; Flow rate: 60 mL/min; Gradient: 48% B to 66% B in 10 min; Wave Length: 254 nm/220 nm) to afford (7R,14R)-1-(difluoromethoxy)-11-(3-methoxyprop-1-yn-1-yl)-6-(methyl-d3)-6,7-dihydro-7,14-methanobenzo[f]benzo[4,5]imidazo[1,2-a][1,4]diazocin-5(14H)-one (5 mg, 10%) as white solid. MS ESI calculated for C₂₃H₁₆D₃F₂N₃O₃ [M+H]⁺, 427.16. found 427.20. ¹H NMR (400 MHz, Chloroform-d) δ 8.51-8.45 (m, 1H), 7.70-7.59 (m, 1H), 7.56 (s, 1H), 7.42 (t, J=8.2 Hz, 1H), 7.36-7.31 (m, 2H), 6.84 (t, J=73.2 Hz, 1H), 6.22 (d, J=6.9 Hz, 1H), 5.02-4.92 (m, 1H), 4.34 (s, 2H), 3.49-3.40 (m, 4H), 2.86 (d, J=13.5 Hz, 1H). ¹⁹F NMR (376 MHz, Chloroform-d) δ −79.98, −80.42, −81.17, −81.61.

### Example 43: (7R,14R)-1-(difluoromethoxy)-11-(3-ethoxyprop-1-yn-1-yl)-6-(methyl-d3)-6,7-dihydro-7,14-methanobenzo[f]benzo[4,5]imidazo[1,2-a][1,4]diazocin-5(14H)-one

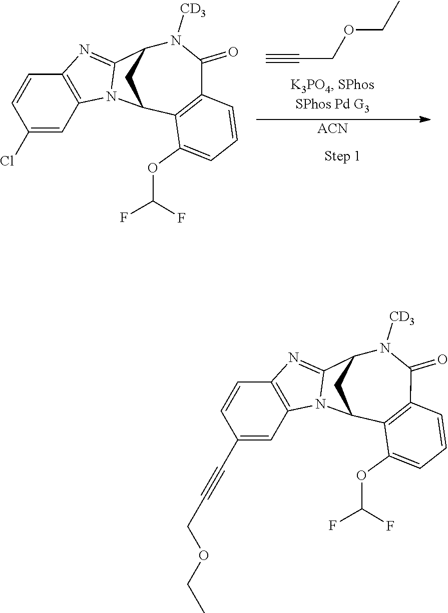

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol), K₂CO₃ (176 mg, 1.275 mmol), SPhos (20 mg, 0.051 mmol), SPhos Pd Gen.3 (40 mg, 0.051 mmol), 3-ethoxyprop-1-yne (107 mg, 1.275 mmol) and ACN (4 mL) at room temperature. The mixture was purged with nitrogen for 3 min and then was stirred for overnight at 80° C. under nitrogen atmosphere. The mixture was concentrated under reduce pressure. The residue was purified by silica gel column chromatography, eluted with CH₂Cl₂/MeOH (12/1) followed by Prep-HPLC with the following conditions (Column: Xselect CSH F-Phenyl OBD column 30*250 mm, 5 μm; Mobile Phase A: Water (0.1% FA), Mobile Phase B: MeOH; Flow rate: 60 mL/min; Gradient: 50% B to 68% B in 10 min; Wave Length: 254 nm/220 nm) to afford (7R,14R)-1-(difluoromethoxy)-11-(3-ethoxyprop-1-yn-1-yl)-6-(methyl-d3)-6,7-dihydro-7,14-methanobenzo[f]benzo[4,5]imidazo[1,2-a][1,4]diazocin-5(14H)-one (25 mg, 22%) as white solid. MS ESI calculated for C₂₄H₁₈D₃F₂N₃O₃ [M+H]⁺, 441.17. found 441.30. ¹H NMR (400 MHz, Chloroform-d) δ 8.50-8.45 (m, 1H), 7.64 (d, J=8.5 Hz, 1H), 7.60-7.54 (m, 1H), 7.43 (t, J=8.2 Hz, 1H), 7.37-7.31 (m, 2H), 6.84 (t, J=73.2 Hz, 1H), 6.23 (d, J=7.1 Hz, 1H), 5.01 (d, J=6.9 Hz, 1H), 4.38 (s, 2H), 3.66 (q, J=7.0 Hz, 2H), 3.52-3.42 (m, 1H), 2.87 (d, J=13.6 Hz, 1H), 1.28 (t, J=7.0 Hz, 3H). ¹⁹F NMR (377 MHz, Chloroform-d) δ −80.02, −80.46, −81.16, −81.61.

### Example 44: (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one

Example 44 refer to the preparation of Example 18, MS ESI calculated for C₂₁H₁₂D₃F₂N₃O₂ [M+H]⁺, 383.13. found 383.15. ¹H NMR (400 MHz, Chloroform-d) δ 8.52-8.46 (m, 1H), 7.65 (d, J=8.4 Hz, 1H), 7.61 (d, J=1.6 Hz, 1H), 7.42 (t, J=8.4 Hz, 1H), 7.39-7.31 (m, 2H), 6.83 (t, J=73.2 Hz, 1H), 6.22 (d, J=7.2 Hz, 1H), 4.96 (d, J=7.2 Hz, 1H), 3.50-3.38 (m, 1H), 3.07 (s, 1H), 2.86 (d, J=13.6 Hz, 1H). ¹⁹F NMR (377 MHz, Chloroform-d) δ −79.96, −80.40, −81.32, −81.76.

### Example 45: (7R,14R)-11-((1,2,4-thiadiazol-5-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

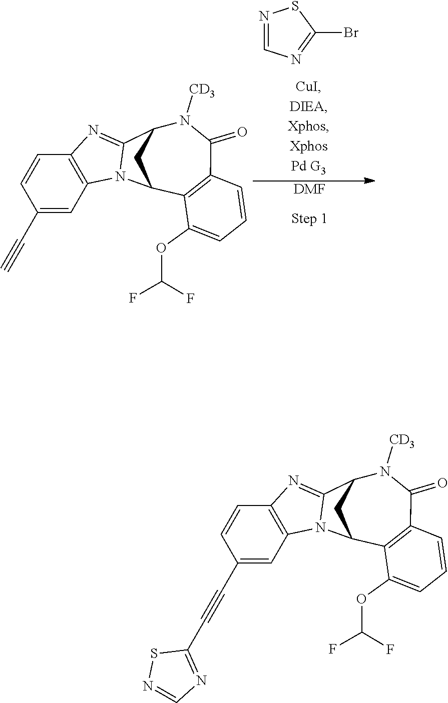

Into a 8 mL vial were added (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), XPhos (6 mg, 0.013 mmol), XPhos Pd G3 (11 mg, 0.013 mmol), DIPEA (114 uL, 0.655 mmol), 5-bromo-1,2,4-thiadiazole (65 mg, 0.393 mmol), CuI (2 mg, 0.013 mmol) and DMF (1 mL) at room temperature. The mixture was purged with nitrogen for 5 min and then was stirred for 5 h at 90° C. The mixture was allowed to cool down to room temperature. The resulting mixture was diluted with water (15 mL) and ethyl acetate (10 mL). The mixture was extracted with ethyl acetate (2×10 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (0% to 10%) followed by Prep-HPLC with the following conditions: column, C18 silica gel; mobile phase, CH₃CN in Water (10 mmol/L NH₄HCO₃), 35% to 70% gradient in 25 min; detector, 254 nm to afford (7R,14R)-11-((1,2,4-thiadiazol-5-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 32%) as a white solid. MS ESI calculated for C₂₃H₁₂D₃F₂N₅O₂S [M+H]⁺, 467.11. found 467.10. ¹H NMR (400 MHz, Chloroform-d) δ 8.74 (s, 1H), 8.50 (d, J=8.0 Hz, 1H), 7.85-7.64 (m, 2H), 7.51 (d, J=7.2 Hz, 1H), 7.45 (t, J=8.1 Hz, 1H), 7.38 (d, J=8.0 Hz, 1H), 6.87 (t, J=72.8 Hz, 1H), 6.36-6.24 (m, 1H), 5.13-4.92 (m, 1H), 3.58-3.39 (m, 1H), 2.91 (d, J=12.6 Hz, 1H). ¹⁹F NMR (376 MHz, Chloroform-d) δ −80.11, −80.55, −81.19, −81.64.

### Example 46: (7R,14R)-1-(difluoromethoxy)-6-(methyl-d3)-11-((5-((methylamino)methyl)-1,3,4-thiadiazol-2-yl)ethynyl)-6,7-dihydro-7,14-methanobenzo[f]benzo[4,5]imidazo[1,2-a][1,4]diazocin-5(14H)-one

### Synthetic Scheme

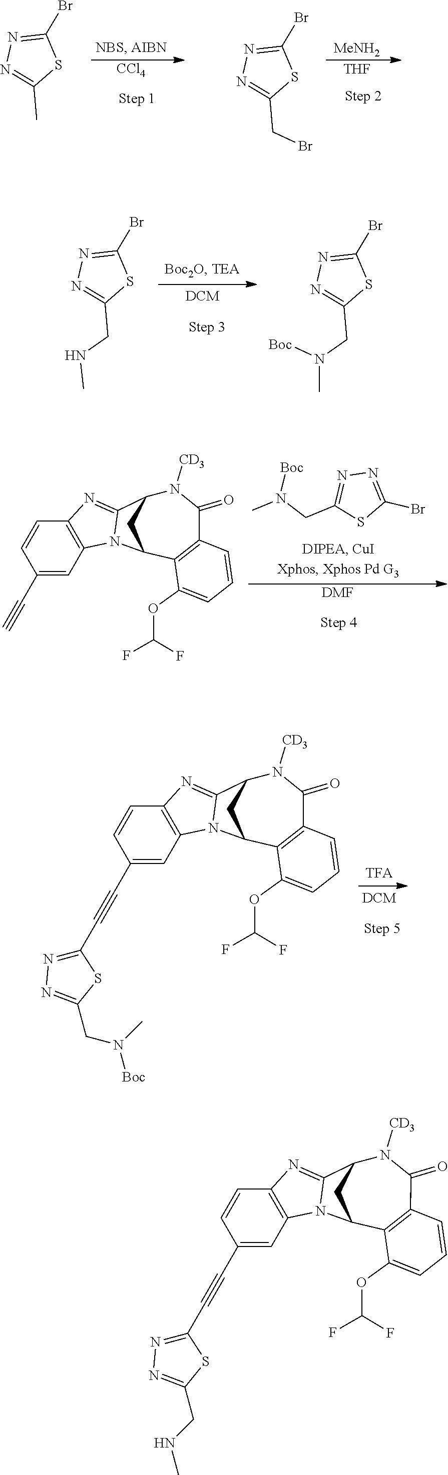

### Preparation 46A: 2-bromo-5-(bromomethyl)-1,3,4-thiadiazole

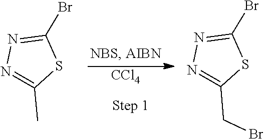

To a solution of 2-bromo-5-methyl-1,3,4-thiadiazole (5.00 g, 27.927 mmol) in CCl₄ (50 mL) was added NBS (5.96 g, 33.512 mmol) and AIBN (0.46 g, 2.793 mmol) at room temperature. The resulting mixture was stirred overnight at 70° C. The mixture was allowed to cool down to room temperature. The resulting mixture was concentrated under vacuum. The residue was purified by silica gel column chromatography, eluted with PE/EA (4/1) to afford 2-bromo-5-(bromomethyl)-1,3,4-thiadiazole (1.50 g, 20%) as yellow solid. MS ESI calculated for C₃H₂Br₂N₂S [M+H]⁺, 256.83 258.83 260.83 found 256.90 258.85 260.85. ¹H NMR (400 MHz, Chloroform-d) δ 4.77 (s, 2H).

### Preparation 46B: 1-(5-bromo-1,3,4-thiadiazol-2-yl)-N-methylmethanamine

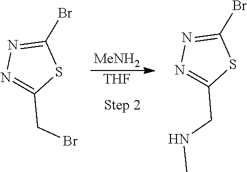

Into a 40 mL vial were added 2-bromo-5-(bromomethyl)-1,3,4-thiadiazole (800 mg, 3.102 mmol) and Methylamine, 2M in THF (10 mL) at room temperature. The resulting mixture was stirred for 1 h at room temperature. The resulting mixture was concentrated under vacuum. The residue was purified by silica gel column chromatography, eluted with PE/EA (1/2) to afford 1-(5-bromo-1,3,4-thiadiazol-2-yl)-N-methylmethanamine (555 mg, 86%) as reddish brown oil. MS ESI calculated for C₄H₆BrN₃S [M+H]⁺, 207.95 209.95. found 208.00 210.05. ¹H NMR (400 MHz, Chloroform-d) δ 4.17 (s, 2H), 2.52 (s, 3H).

### Preparation 46C: tert-butyl ((5-bromo-1,3,4-thiadiazol-2-yl)methyl)(methyl)carbamate

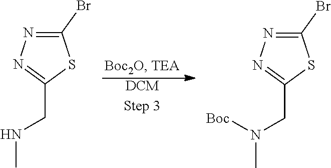

To a solution of 1-(5-bromo-1,3,4-thiadiazol-2-yl)-N-methylmethanamine (200 mg, 0.961 mmol) in DCM (3.00 mL) was added Boc₂O (251 mg, 1.153 mmol) and TEA (194 mg, 1.922 mmol) at room temperature. The resulting mixture was stirred for 1 h at room temperature. The resulting mixture was concentrated under vacuum. The residue was purified by silica gel column chromatography, eluted with PE/EA (8/1) to afford tert-butyl ((5-bromo-1,3,4-thiadiazol-2-yl)methyl)(methyl)carbamate (150 mg, 50%) as brown oil. MS ESI calculated for C₉H₁₄BrN₃O₂S [M+H]⁺, 308.00 310.00. found 308.00, 310.00. ¹H NMR (400 MHz, Chloroform-d) δ 4.74 (s, 2H), 2.94 (s, 3H), 1.81-1.18 (m, 9H).

### Preparation 46D: tert-butyl ((5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)methyl)(methyl)carbamate

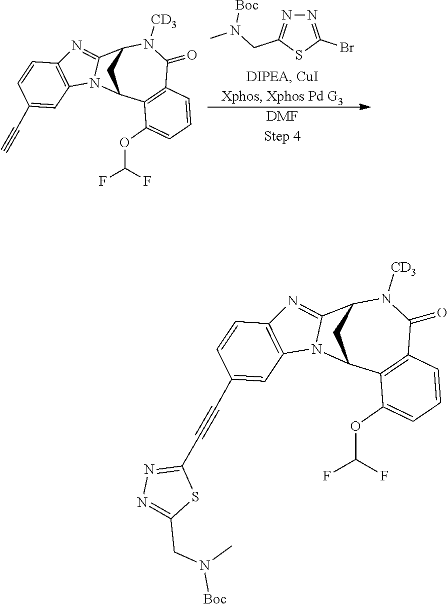

Into a 8 mL vial were added (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (60 mg, 0.157 mmol), tert-butyl ((5-bromo-1,3,4-thiadiazol-2-yl)methyl)(methyl)carbamate (145 mg, 0.471 mmol), XPhos (7 mg, 0.016 mmol), XPhos Pd G₃ (13 mg, 0.016 mmol), CuI (3 mg, 0.016 mmol), DIEA (101 mg, 0.785 mmol) and DMF (1 mL) at room temperature. The mixture was purged with nitrogen for 3 min and then was stirred for 4 h at 100° C. under nitrogen atmosphere. The mixture was allowed to cool down to room temperature. The residue was purified by silica gel column chromatography, eluted with CH₂Cl₂/MeOH (9/1) to afford tert-butyl ((5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)methyl)(methyl)carbamate (74 mg, 77%) as brown oil. MS ESI calculated for C₃₀H₂₅D₃F₂N₆O₄S [M+H]⁺, 610.20. found 610.35. ¹H NMR (400 MHz, Chloroform-d) δ 8.50 (d, J=8.2 Hz, 1H), 7.75 (s, 1H), 7.57-7.50 (m, 1H), 7.49-7.36 (m, 2H), 7.32-7.23 (m, 1H), 6.78 (t, J=72.4 Hz, 1H), 6.34-6.24 (m, 1H), 5.12-5.00 (m, 1H), 4.81 (s, 2H), 3.52-3.41 (m, 1H), 3.03-2.85 (m, 4H), 1.51 (s, 9H).

### Example 46: (7R,14R)-1-(difluoromethoxy)-6-(methyl-d3)-11-((5-((methylamino)methyl)-1,3,4-thiadiazol-2-yl)ethynyl)-6,7-dihydro-7,14-methanobenzo[f]benzo[4,5]imidazo[1,2-a][1,4]diazocin-5(14H)-one

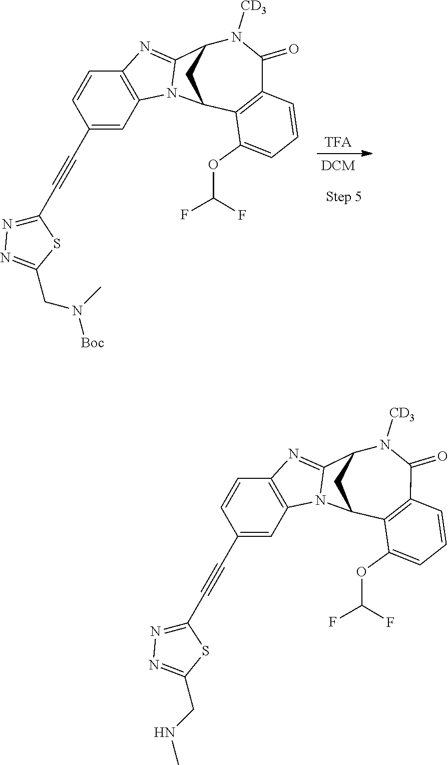 [SMILES: [2H]C([2H])([2H])N1C(=O)c2cccc(OC(F)F)c2[C@H]2C[C@@H]1c1nc3ccc(C#Cc4nnc(CN(C)C(=O)OC(C)(C)C)s4)cc3n12 ; [2H]C([2H])([2H])N1C(=O)c2cccc(OC(F)F)c2[C@H]2C[C@@H]1c1nc3ccc(C#Cc4nnc(CNC)s4)cc3n12]

A solution of tert-butyl ((5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)methyl)(methyl)carbamate (74 mg, 0.121 mmol) in DCM (5 mL) and TFA (1 mL) was stirred for 2 h at room temperature. The resulting mixture was concentrated under reduced pressure. The residue was purified by Prep-HPLC with the following conditions (Column: XBridge Prep OBD C18 Column 30*150 mm, 5m; Mobile Phase A: Water (0.1% FA), Mobile Phase B: ACN; Flow rate: 60 mL/min; Gradient: 15% B to 25% B in 10 min; Wave Length: 254 nm/220 nm) to afford (7R,14R)-1-(difluoromethoxy)-6-(methyl-d3)-11-((5-((methylamino)methyl)-1,3,4-thiadiazol-2-yl)ethynyl)-6,7-dihydro-7,14-methanobenzo[f]benzo[4,5]imidazo[1,2-a][1,4]diazocin-5(14H)-one (32 mg, 52%) as white solid. MS ESI calculated for C₂₅H₁₇D₃F₂N₆O₂S [M+H]⁺, 510.15. found 510.35. ¹H NMR (400 MHz, Chloroform-d) δ 8.51-8.47 (m, 1H), 7.73-7.66 (m, 2H), 7.49-7.34 (m, 3H), 6.87 (t, J=72.6 Hz, 1H), 6.23 (d, J=7.3 Hz, 1H), 4.96 (d, J=6.9 Hz, 1H), 4.29 (s, 2H), 3.55-3.41 (m, 1H), 2.88 (d, J=13.6 Hz, 1H), 2.29 (s, 3H). ¹⁹F NMR (377 MHz, Chloroform-d) δ −80.07, −80.51, −81.35, −81.80.

### Example 47: (7R,14R)-1-(difluoromethoxy)-6-(methyl-d3)-11-(piperidin-2-ylethynyl)-6,7-dihydro-7,14-methanobenzo[f]benzo[4,5]imidazo[1,2-a][1,4]diazocin-5(14H)-one

### Synthetic Scheme

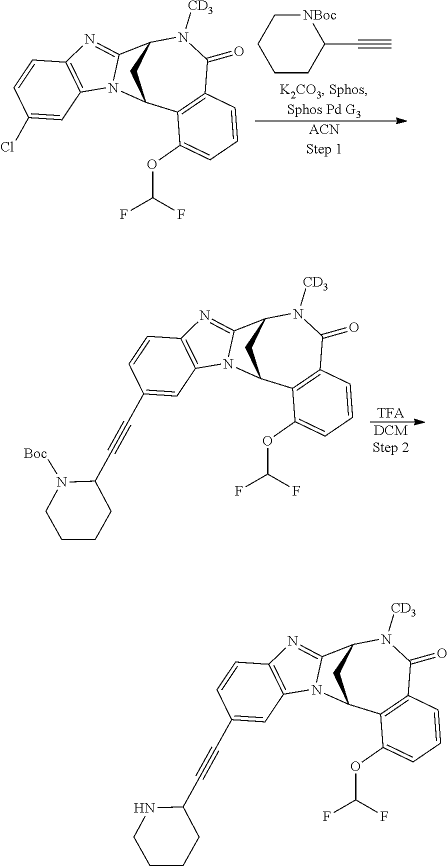

### Preparation 47A: tert-butyl 2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate

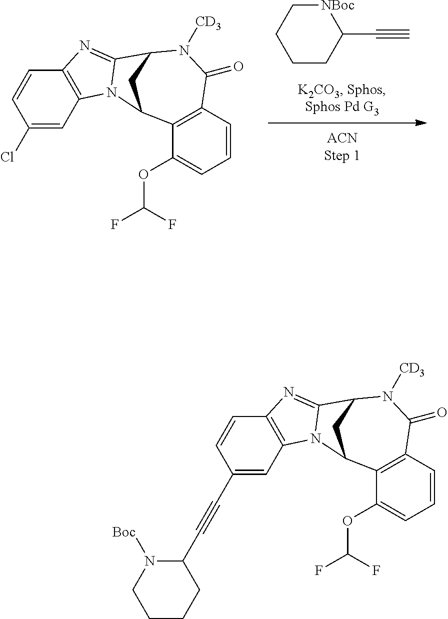

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol), tert-butyl 2-ethynylpiperidine-1-carboxylate (266 mg, 1.275 mmol), K₂CO₃ (175 mg, 1.275 mmol), SPhos (20 mg, 0.051 mmol), SPhos Pd G₃(40 mg, 0.051 mmol) and ACN (2.5 mL) at room temperature. The mixture was purged with nitrogen for 3 min and then was stirred for 1 h at 100° C. under nitrogen atmosphere. The mixture was concentrated under reduce pressure. The residue was purified by silica gel column chromatography, eluted with CH₂Cl₂/MeOH (10/1) to afford tert-butyl 2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (130 mg, 90%) as yellow solid. MS ESI calculated for C₃₁H₂₉D₃F₂N₄O₄[M+H]⁺, 566.26 found 566.45. ¹H NMR (400 MHz, Chloroform-d) δ 8.55-8.44 (m, 1H), 7.80-7.61 (m, 1H), 7.56-7.49 (m, 1H), 7.46-7.39 (m, 1H), 7.36-7.30 (m, 2H), 7.07-6.58 (m, 1H), 6.31-6.17 (m, 1H), 5.12-4.95 (m, 1H), 3.97 (d, J=11.5 Hz, 1H), 3.73-3.64 (m, 1H), 3.57-3.38 (m, 1H), 3.12 (t, J=13.2 Hz, 1H), 2.93-2.79 (m, 1H), 1.90-1.59 (m, 6H), 1.49 (s, 9H).

### Example 47: (7R,14R)-1-(difluoromethoxy)-6-(methyl-d3)-11-(piperidin-2-ylethynyl)-6,7-dihydro-7,14-methanobenzo[f]benzo[4,5]imidazo[1,2-a][1,4]diazocin-5(14H)-one

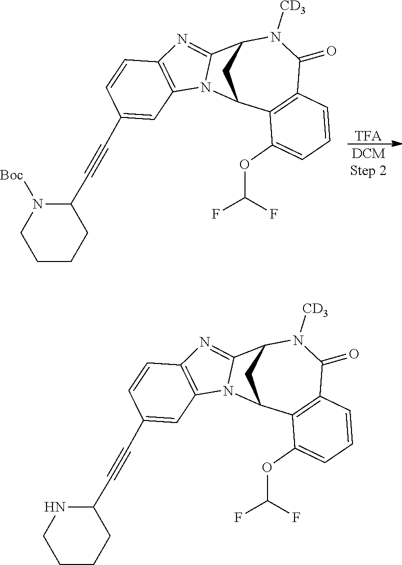 [SMILES: [2H]C([2H])([2H])N1C(=O)c2cccc(OC(F)F)c2[C@H]2C[C@@H]1c1nc3ccc(C#CC4CCCCN4C(=O)OC(C)(C)C)cc3n12 ; [2H]C([2H])([2H])N1C(=O)c2cccc(OC(F)F)c2[C@H]2C[C@@H]1c1nc3ccc(C#CC4CCCCN4)cc3n12]

A solution of tert-butyl 2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (130 mg, 0.230 mmol) in DCM (5 mL) and TFA (1 mL) was stirred for 2 h at room temperature. The resulting mixture was concentrated under reduced pressure. The residue was purified by Prep-HPLC with the following conditions (Column: XBridge Prep OBD C18 Column 30*150 mm, 5m; Mobile Phase A: Water (0.1% FA), Mobile Phase B: ACN; Flow rate: 60 mL/min; Gradient: 26% B to 44% B in 10 min; Wave Length: 254 nm/220 nm; RT1(min): 11.12) to afford (7R,14R)-1-(difluoromethoxy)-6-(methyl-d3)-11-(piperidin-2-ylethynyl)-6,7-dihydro-7,14-methanobenzo[f]benzo[4,5]imidazo[1,2-a][1,4]diazocin-5(14H)-one (39 mg, 36%) as white solid. MS ESI calculated for C₂₆H₂₁D₃F₂N₄O₂ [M+H]⁺, 466.21 found 466.35. ¹H NMR (400 MHz, Methanol-d₄) δ 8.53 (d, J=2.4 Hz, 1H), 8.42-8.28 (m, 1H), 7.73-7.57 (m, 2H), 7.55-7.00 (m, 4H), 6.36 (d, J=7.2 Hz, 1H), 5.19 (d, J=7.1 Hz, 1H), 4.34-4.21 (m, 1H), 3.62-3.50 (m, 1H), 3.05-2.95 (m, 1H), 2.89 (d, J=13.9 Hz, 1H), 2.19-2.07 (m, 1H), 2.00-1.59 (m, 5H). ¹⁹F NMR (376 MHz, Methanol-d₄) δ −82.33, −82.80, −83.62, −84.07.

### Example 48: (7R,14R)-1-(difluoromethoxy)-6-(methyl-d3)-11-((tetrahydro-2H-pyran-4-yl)ethynyl)-6,7-dihydro-7,14-methanobenzo[f]benzo[4,5]imidazo[1,2-a][1,4]diazocin-5(14H)-one

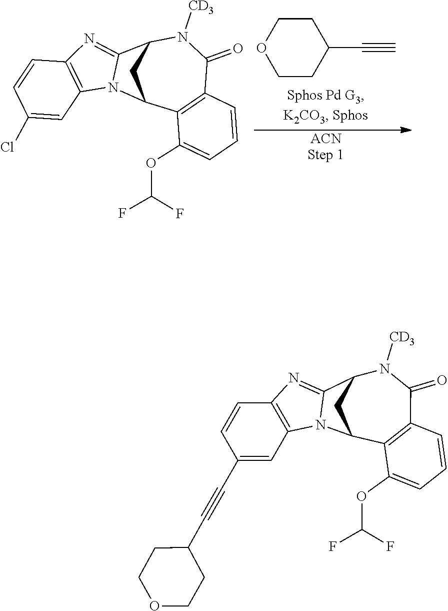

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and 4-ethynyloxane (50 mg, 0.459 mmol) in ACN (1 mL) were added K₂CO₃ (105 mg, 0.765 mmol), SPhos Pd Gen.3 (24 mg, 0.031 mmol) and SPhos (12 mg, 0.031 mmol) in portions at room temperature under nitrogen atmosphere. The resulting mixture was stirred for overnight at 80° C. under nitrogen atmosphere. The resulting mixture was concentrated under reduced pressure. The residue was purified by silica gel column chromatography, eluted with CH₂Cl₂/MeOH (0% to 10%) followed by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 27% B to 45% B in 10 min; Wave Length: 254/220 nm;) to afford (7R,14R)-1-(difluoromethoxy)-6-(methyl-d3)-11-((tetrahydro-2H-pyran-4-yl)ethynyl)-6,7-dihydro-7,14-methanobenzo[f]benzo[4,5]imidazo[1,2-a][1,4]diazocin-5(14H)-one (17 mg, 23%) as a white solid. MS ESI calculated for C₂₆H₂₀D₃F₂N₃O₃[M+H]⁺, 467.19. found 467.30. ¹H NMR (400 MHz, Chloroform-d) δ 8.51-8.41 (m, 1H), 7.62 (d, J=8.4 Hz, 1H), 7.52 (s, 1H), 7.42 (t, J=8.4 Hz, 1H), 7.35-7.27 (m, 2H), 7.03-6.65 (m, 1H), 6.21 (d, J=7.0 Hz, 1H), 5.01-4.89 (m, 1H), 4.00-3.91 (m, 2H), 3.61-3.52 (m, 2H), 3.50-3.39 (m, 1H), 2.93-2.81 (m, 2H), 1.98-1.88 (m, 2H), 1.80-1.74 (m, 2H). ¹⁹F NMR (377 MHz, Chloroform-d) δ −80.00, −80.45, −81.14, −81.58.

### Example 49: (7R,14R)-1-(difluoromethoxy)-6-(methyl-d3)-11-((tetrahydro-2H-pyran-2-yl)ethynyl)-6,7-dihydro-7,14-methanobenzo[f]benzo[4,5]imidazo[1,2-a][1,4]diazocin-5(14H)-one

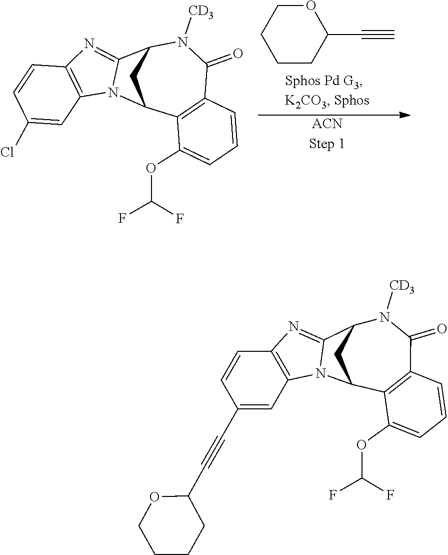

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and 2-ethynyloxane (67 mg, 0.612 mmol) in ACN (1 mL) were added K₂CO₃ (141 mg, 1.020 mmol), SPhos Pd Gen.3 (32 mg, 0.041 mmol) and SPhos (17 mg, 0.041 mmol) in portions at room temperature under nitrogen atmosphere. The resulting mixture was stirred for overnight at 80° C. under nitrogen atmosphere. The residue was purified by reversed-phase flash chromatography with the following conditions: column, C18 silica gel; mobile phase, CH₃CN in Water (10 mmol/L NH₄HCO₃), 40% to 70% gradient in 20 min; detector, 254/220 nm followed by Prep-HPLC with the following conditions (Column: XBridge Shield RP18 OBD Column 30*150 mm, 5m; Mobile Phase A: Water (10 mmol/L NH₄HCO₃+0.1% NH₃·H₂O), Mobile Phase B: MeOH; Flow rate: 60 mL/min; Gradient: 47% B to 67% B in 10 min; Wave Length: 254/220 nm; RT1(min): 11.83) to afford (7R,14R)-1-(difluoromethoxy)-6-(methyl-d3)-11-((tetrahydro-2H-pyran-2-yl)ethynyl)-6,7-dihydro-7,14-methanobenzo[f]benzo[4,5]imidazo[1,2-a][1,4]diazocin-5(14H)-one (5 mg, 5%) as a white solid. MS ESI calculated for C₂₆H₂₀D₃F₂N₃O₃ [M+H]⁺, 467.19. found 467.25. ¹H NMR (400 MHz, Chloroform-d) δ 8.52-8.45 (m, 1H), 7.64 (d, J=8.5 Hz, 1H), 7.57 (s, 1H), 7.43 (t, J=8.2 Hz, 1H), 7.37-7.32 (m, 2H), 7.03-6.65 (m, 1H), 6.27-6.16 (m, 1H), 5.05-4.91 (m, 1H), 4.55-4.48 (m, 1H), 4.10-4.01 (m, 1H), 3.64-3.55 (m, 1H), 3.52-3.38 (m, 1H), 2.87 (d, J=11.1 Hz, 1H), 1.91-1.86 (m, 2H), 1.85-1.78 (m, 1H), 1.67-1.55 (m, 3H). ¹⁹F NMR (377 MHz, Chloroform-d) δ −80.00, −80.44, −81.31, −81.75.

### Example 50: (7R,14R)-11-((R)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

### Synthetic Scheme

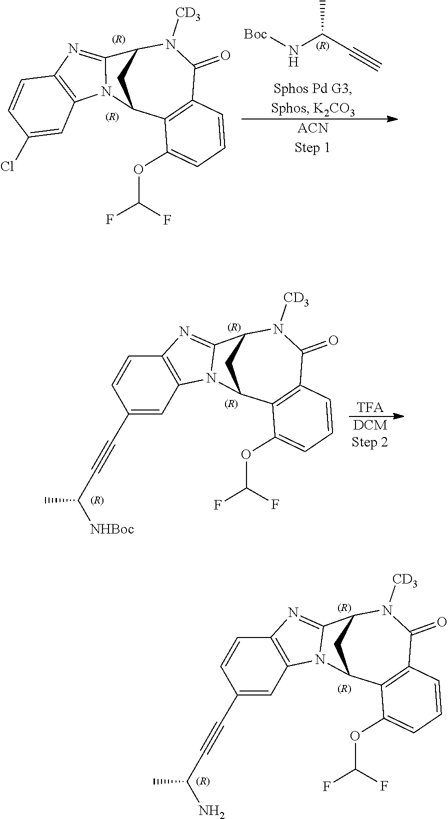

### Preparation 50A: tert-butyl ((R)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate

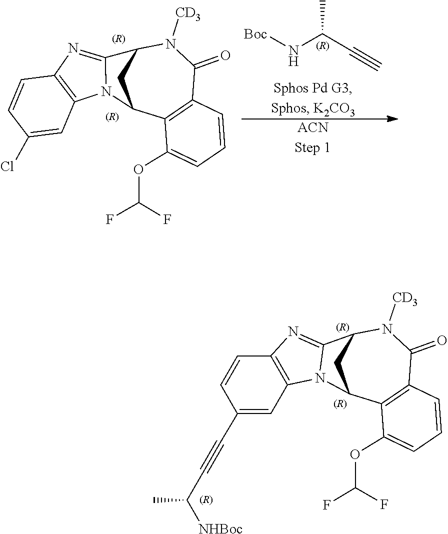

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol), tert-butyl (R)-but-3-yn-2-ylcarbamate (323 mg, 1.910 mmol), K₂CO₃ (264 mg, 1.910 mmol), SPhos Pd Gen.3 (60 mg, 0.076 mmol) and SPhos (31 mg, 0.076 mmol) in ACN (1.5 mL) was stirred for 4 h at 80° C. under nitrogen atmosphere. The mixture was allowed to cool down to room temperature. The resulting mixture was concentrated under reduced pressure. The residue was purified by silica gel column chromatography, eluted with CH₂Cl₂/MeOH (10/1) to afford tert-butyl ((R)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate (200 mg, crude) as yellow solid. MS ESI calculated for C₂₈H₂₅D₃F₂N₄O₄ [M+H]⁺, 526.23. found 526.20.

### Example 50: (7R,14R)-11-((R)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

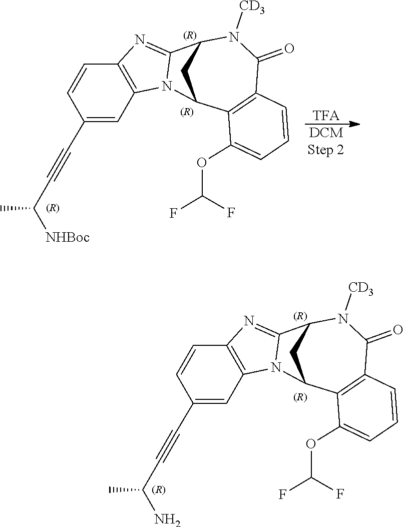

A solution of tert-butyl ((R)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate (200 mg, 0.381 mmol) in DCM (5 mL) and TFA (1 mL) was stirred for 2 h at room temperature. The resulting mixture was concentrated under reduced pressure. The residue was purified by reversed-phase flash chromatography with the following conditions: column, C18 silica gel; mobile phase, CH₃CN in water (10 mmol/L NH₄HCO₃), 30% to 45% gradient in 10 min; detector, 254 nm to afford (7R,14R)-11-((R)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (4 mg, 3%) as a brown yellow solid. MS ESI calculated for C₂₃H₁₇D₃F₂N₄O₂ [M+H]⁺, 426.17. found 426.20. ¹H NMR (400 MHz, Chloroform-d) δ 8.51-8.38 (m, 1H), 7.64-7.46 (m, 2H), 7.46-7.27 (m, 3H), 7.00-6.66 (m, 1H), 6.16 (d, J=7.5 Hz, 1H), 4.98-4.87 (m, 1H), 4.05-3.95 (m, 1H), 3.47-3.33 (m, 1H), 2.89-2.67 (m, 1H), 1.57-1.45 (m, 3H). ¹⁹F NMR (377 MHz, Chloroform-d) δ −80.09, −80.38, −80.53, −81.24, −81.26, −81.43, −81.70, −81.87.

### Example 51: (7R,14R)-11-((S)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

### Synthetic Scheme

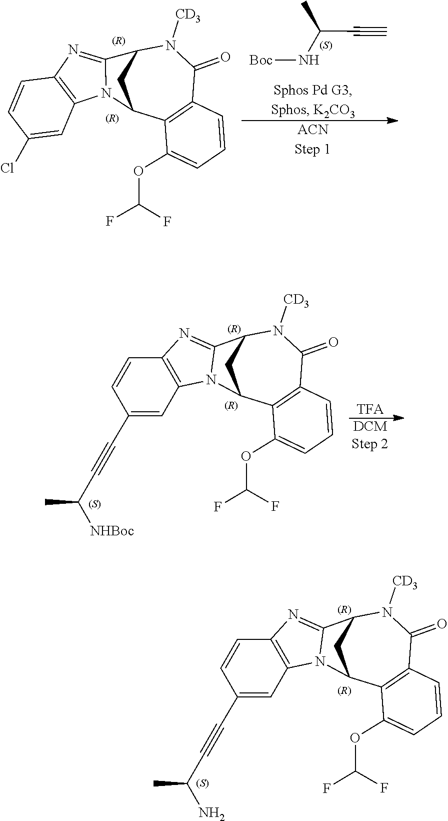

### Preparation 51A: tert-butyl ((S)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate

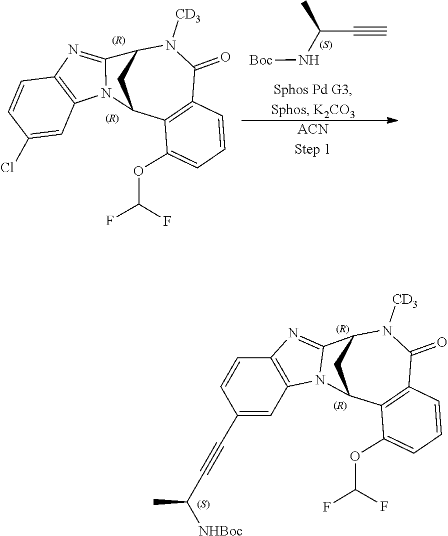

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol), tert-butyl (S)-but-3-yn-2-ylcarbamate (323 mg, 1.910 mmol), K₂CO₃ (264 mg, 1.910 mmol), SPhos Pd Gen.3 (60 mg, 0.076 mmol), SPhos (31 mg, 0.076 mmol) and ACN (1.5 mL) at room temperature. The resulting mixture was stirred for 4 h at 80° C. under nitrogen atmosphere. The mixture was allowed to cool down to room temperature. The resulting mixture was concentrated under reduced pressure. The residue was purified by silica gel column chromatography, eluted with CH₂Cl₂/MeOH (10/1) to afford tert-butyl ((S)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate (150 mg, crude) as a yellow solid. MS ESI calculated for C₂₈H₂₅D₃F₂N₄O₄ [M+H]⁺, 526.23. found 526.40.

### Example 51: (7R,14R)-11-((S)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

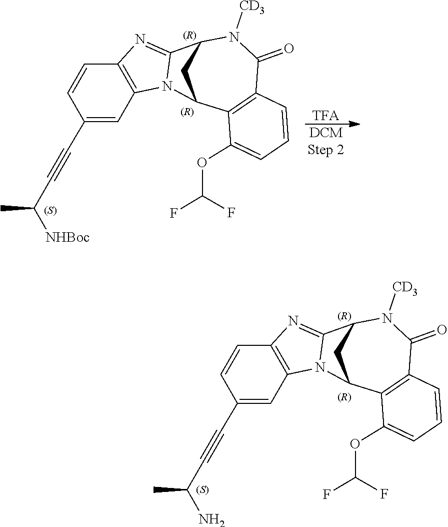

A solution of tert-butyl ((S)-4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-2-yl)carbamate (150 mg, 0.571 mmol) in DCM (5 mL) and TFA (1 mL) was stirred for 2 h at room temperature. The resulting mixture was concentrated under reduced pressure. The residue was purified by reversed-phase flash chromatography with the following conditions: column, C18 silica gel; mobile phase, CH₃CN in water (10 mmol/L NH₄HCO₃), 35% to 45% gradient in 10 min; detector, 254 nm to afford (7R,14R)-11-((S)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (10 mg, 8%) as yellow solid. MS ESI calculated for C₂₃H₁₇D₃F₂N₄O₂ [M+H]⁺, 426.17. found 426.15. ¹H NMR (400 MHz, Chloroform-d) δ 8.50-8.45 (m, 1H), 7.61 (d, J=8.5 Hz, 1H), 7.49 (t, J=1.1 Hz, 1H), 7.40 (t, J=8.2 Hz, 1H), 7.37-7.31 (m, 1H), 7.28 (d, J=1.5 Hz, 1H), 7.07-6.65 (m, 1H), 6.16 (d, J=7.2 Hz, 1H), 4.92 (d, J=7.1 Hz, 1H), 4.00 (d, J=7.4 Hz, 1H), 3.47-3.36 (m, 1H), 2.84 (d, J=13.5 Hz, 1H), 1.49 (d, J=6.7 Hz, 3H). ¹⁹F NMR (377 MHz, Chloroform-d) δ −79.94, −79.97, −80.38, −80.41, −81.25, −81.28, −81.70, −81.72.

### Example 52: (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one

### Synthetic Scheme

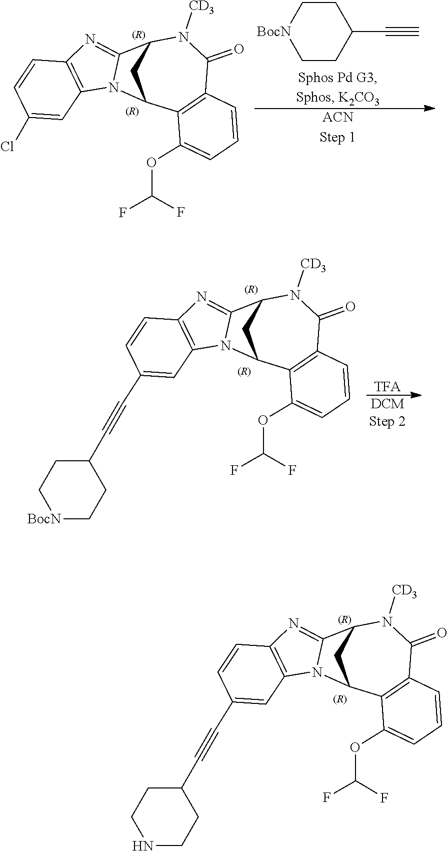

### Preparation 52A: tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate

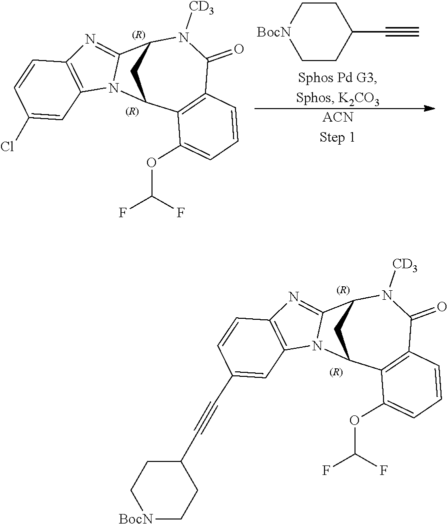

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), tert-butyl 4-ethynylpiperidine-1-carboxylate (160 mg, 0.765 mmol), K₂CO₃ (106 mg, 0.765 mmol), SPhos Pd Gen.3 (24 mg, 0.031 mmol) and SPhos (13 mg, 0.031 mmol) in ACN (1.5 mL) was stirred for 3 h at 80° C. under nitrogen atmosphere. The mixture was concentrated under reduce pressure. The residue was purified by silica gel column chromatography, eluted with CH₂Cl₂/MeOH (7/1) to afford tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (90 mg, crude) as a yellow solid. MS ESI calculated for C₃₁H₂₉D₃F₂N₄O₄[M+H]⁺, 566.26. found 566.30.

### Example 52: (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one

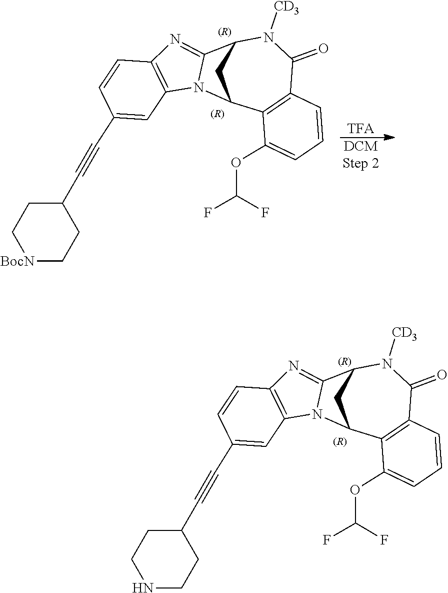

A solution of tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (90 mg, 0.159 mmol) in DCM (5 mL) and TFA (1 mL) was stirred for 2 h at room temperature. The resulting mixture was concentrated under reduced pressure. The residue was purified by reversed-phase flash chromatography with the following conditions: column, C18 silica gel; mobile phase, CH₃CN in water (10 mmol/L NH₄HCO₃), 40% to 50% gradient in 10 min; detector, 254 nm to afford (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (10 mg, 13%) as a white solid. MS ESI calculated for C₂₆H₂₁D₃F₂N₄O₂ [M+H]⁺, 466.21. found 466.05. ¹H NMR (300 MHz, Methanol-d₄) δ 8.39-8.33 (m, 1H), 7.61-6.97 (m, 6H), 6.34 (d, J=7.2 Hz, 1H), 5.17 (d, J=7.2 Hz, 1H), 3.62-3.48 (m, 1H), 3.21-3.11 (m, 2H), 2.92-2.74 (m, 4H), 2.05-1.91 (m, 2H), 1.83-1.66 (m, 2H). ¹⁹F NMR (282 MHz, Methanol-d₄) δ −82.89, −83.53, −83.56, −84.22.

### Example 53: (7R,14R)-1-(difluoromethoxy)-6-(methyl-d3)-11-((tetrahydrofuran-2-yl)ethynyl)-6,7-dihydro-7,14-methanobenzo[f]benzo[4,5]imidazo[1,2-a][1,4]diazocin-5(14H)-one

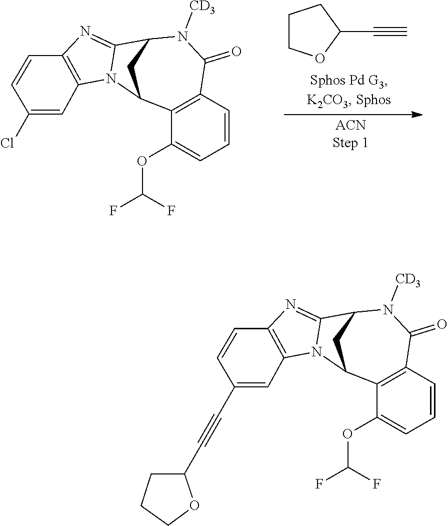

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (140 mg, 0.356 mmol) and 2-ethynyloxolane (103 mg, 1.068 mmol) in ACN (2 mL) were added K₂CO₃ (246 mg, 1.780 mmol), SPhos Pd Gen.3 (55 mg, 0.071 mmol) and SPhos (29 mg, 0.071 mmol) in portions at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by reversed-phase flash chromatography with the following conditions: column, C18 silica gel; mobile phase, CH₃CN in Water (10 mmol/L NH₄HCO₃), 40% to 70% gradient in 20 min; detector, 254/220 nm followed by Prep-HPLC with the following conditions (Column: XBridge Shield RP18 OBD Column 30*150 mm, 5m; Mobile Phase A: Water (10 mmol/L NH₄HCO₃), Mobile Phase B: MeOH; Flow rate: 60 mL/min; Gradient: 43% B to 63% B in 12 min; Wave Length: 254/200 nm) to afford (7R,14R)-1-(difluoromethoxy)-6-(methyl-d3)-11-((tetrahydrofuran-2-yl)ethynyl)-6,7-dihydro-7,14-methanobenzo[f]benzo[4,5]imidazo[1,2-a][1,4]diazocin-5(14H)-one (5 mg, 5%) as a white solid. MS ESI calculated for C₂₅H₁₈D₃F₂N₃O₃ [M+H]⁺, 453.17. found 453.20. ¹H NMR (400 MHz, Methanol-d₄) δ 8.42-8.30 (m, 1H), 7.60-7.53 (m, 2H), 7.51-7.40 (m, 2H), 7.31-7.04 (m, 2H), 6.41-6.24 (m, 1H), 5.20-5.13 (m, 1H), 4.81 (t, J=6.0 Hz, 1H), 4.04-3.93 (m, 1H), 3.88-3.80 (m, 1H), 3.59-3.48 (m, 1H), 2.91-2.82 (m, 1H), 2.33-2.20 (m, 1H), 2.18-1.85 (m, 3H). ¹⁹F NMR (377 MHz, Methanol-d₄) δ −83.57.

### Example 54: (7R,14R)-1-(difluoromethoxy)-11-((R)-3-(ethylamino)but-1-yn-1-yl)-6-(methyl-d3)-6,7-dihydro-7,14-methanobenzo[f]benzo[4,5]imidazo[1,2-a][1,4]diazocin-5(14H)-one

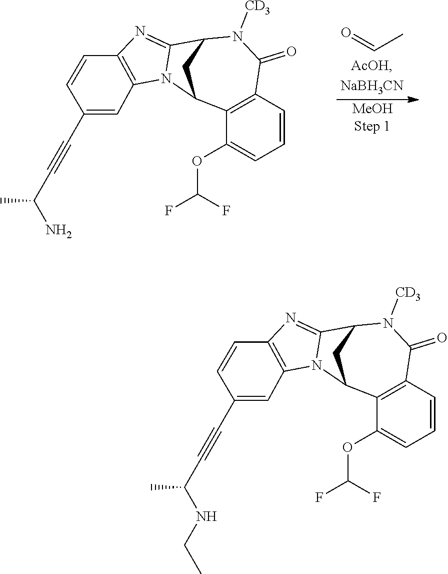

To a stirred solution of (7R,14R)-11-((R)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (30 mg, 0.071 mmol) in MeOH (1 mL) were added acetaldehyde (3 mg, 0.071 mmol) and AcOH (13 mg, 0.213 mmol) in portions at room temperature. The resulting mixture was stirred for 20 min at room temperature. To the above mixture was added NaBH₃CN (7 mg, 0.106 mmol) in portions over 5 min at 0° C. The resulting mixture was stirred for additional 2 h at room temperature. The resulting mixture was purified by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 20% B to 40% B in 10 min; Wave Length: 254 nm/220 nm; RT1(min): 10.72) to afford (7R,14R)-1-(difluoromethoxy)-11-((R)-3-(ethylamino)but-1-yn-1-yl)-6-(methyl-d3)-6,7-dihydro-7,14-methanobenzo[f]benzo[4,5]imidazo[1,2-a][1,4]diazocin-5(14H)-one (7 mg, 22%) as white solid. MS ESI calculated for C₂₅H₂₁D₃F₂N₄O₂ [M+H]⁺, 454.21 found 454.20. ¹H NMR (400 MHz, Chloroform-d) δ 8.50-8.45 (m, 1H), 7.64-7.60 (m, 1H), 7.55-7.52 (m, 1H), 7.42 (t, J=8.2 Hz, 1H), 7.36-7.27 (m, 2H), 6.87 (t, J=73.3 Hz, 1H), 6.21 (d, J=7.2 Hz, 1H), 4.93 (d, J=7.1 Hz, 1H), 3.88-3.78 (m, 1H), 3.48-3.38 (m, 1H), 3.11-2.99 (m, 1H), 2.91-2.71 (m, 2H), 1.54-1.48 (m, 3H), 1.27-1.17 (m, 3H). ¹⁹F NMR (377 MHz, Chloroform-d) δ −80.07, −80.51, −81.25, −81.69.

### Example 55: (7R,14R)-1-(difluoromethoxy)-11-((S)-3-(ethylamino)but-1-yn-1-yl)-6-(methyl-d3)-6,7-dihydro-7,14-methanobenzo[f]benzo[4,5]imidazo[1,2-a][1,4]diazocin-5(14H)-one

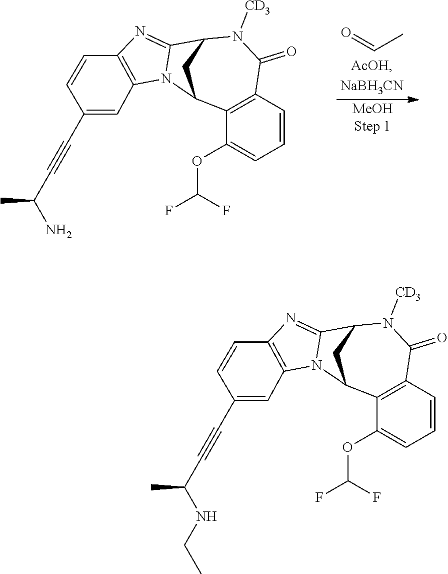

To a stirred solution of (7R,14R)-11-((S)-3-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (29 mg, 0.068 mmol) in MeOH (1 mL) were added acetaldehyde (3 mg, 0.068 mmol) and AcOH (12 mg, 0.204 mmol) in portions at room temperature. The resulting mixture was stirred for 20 min at room temperature. To the above mixture was added NaBH₃CN (6 mg, 0.102 mmol) in portions over 5 min at 0° C. The resulting mixture was stirred for additional 2 h at room temperature. The resulting solution was purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min; Gradient: 27% B to 44% B in 10 min; Wave Length: 254 nm/220 nm; RT1(min): 9.73) to afford (7R,14R)-1-(difluoromethoxy)-11-((S)-3-(ethylamino)but-1-yn-1-yl)-6-(methyl-d3)-6,7-dihydro-7,14-methanobenzo[f]benzo[4,5]imidazo[1,2-a][1,4]diazocin-5(14H)-one (14 mg, 42%) as a white solid. MS ESI calculated for C₂₅H₂₁D₃F₂N₄O₂ [M+H]⁺, 454.21 found 454.20. ¹H NMR (400 MHz, Chloroform-d) δ 8.52-8.45 (m, 1H), 7.62 (d, J=8.4 Hz, 1H), 7.53 (s, 1H), 7.43 (d, J=8.2 Hz, 1H), 7.33 (d, J=8.1 Hz, 1H), 7.30-7.27 (m, 1H), 6.87 (t, J=72.7 Hz, 1H), 6.20 (d, J=7.2 Hz, 1H), 4.93 (d, J=7.2 Hz, 1H), 3.91-3.76 (m, 1H), 3.49-3.39 (m, 1H), 3.12-3.00 (m, 1H), 2.89-2.73 (m, 2H), 1.58-1.47 (m, 3H), 1.29-1.17 (m, 3H). ¹⁹F NMR (377 MHz, Chloroform-d) δ −80.05, −80.50, −81.26, −81.71.

### Example 56: (7R,14R)-11-(3-(tert-butoxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

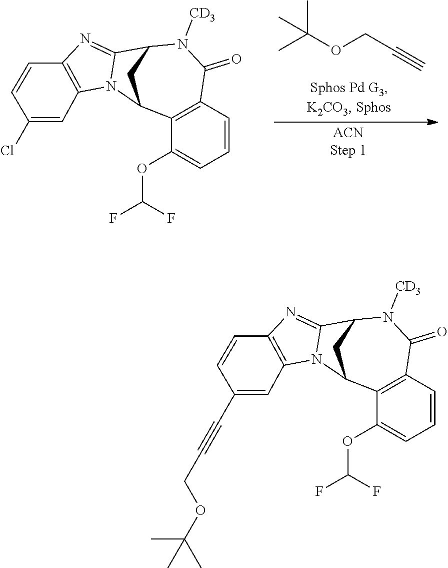

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and 3-(tert-butoxy)prop-1-yne (51 mg, 0.459 mmol) in ACN (1.0 mL) were added K₂CO₃ (105 mg, 0.765 mmol), SPhos Pd Gen.3 (23 mg, 0.031 mmol) and SPhos (12 mg, 0.031 mmol) in portions at room temperature under nitrogen atmosphere. The resulting mixture was stirred for overnight at 80° C. under nitrogen atmosphere. The resulting mixture was concentrated under vacuum. The residue was purified by silica gel column chromatography, eluted with DCM/MeOH (0% to 10%) followed by reversed-phase flash chromatography with the following conditions: column, C18 silica gel; mobile phase, CH₃CN in Water (10 mmol/L NH₄HCO₃), 40% to 70% gradient in 20 min; detector, 254/220 nm to afford (7R,14R)-11-(3-(tert-butoxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (4 mg, 4%) as a white solid. MS ESI calculated for C₂₆H₂₂D₃F₂N₃O₃[M+H]⁺, 469.21. found 469.20. ¹H NMR (400 MHz, Methanol-d₄) δ 8.53-8.23 (m, 1H), 7.62-7.53 (m, 2H), 7.51-7.40 (m, 2H), 7.31-7.02 (m, 2H), 6.43-6.26 (m, 1H), 5.22-5.07 (m, 1H), 4.60-4.31 (m, 2H), 3.65-3.46 (m, 1H), 2.98-2.76 (m, 1H), 1.29-1.27 (m, 9H). ¹⁹F NMR (377 MHz, Methanol-d₄) δ −82.97, −83.43, −83.48, −83.92.

### Example 57: (7R,14R)-1-(difluoromethoxy)-6-(methyl-d3)-11-((1-methylpiperidin-4-yl)ethynyl)-6,7-dihydro-7,14-methanobenzo[f]benzo[4,5]imidazo[1,2-a][1,4]diazocin-5(14H)-one

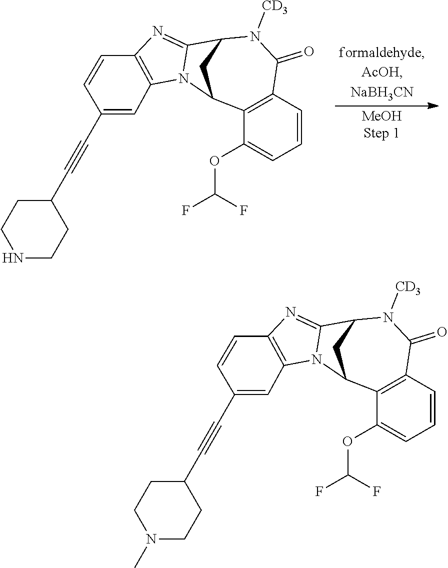 [SMILES: [2H]C([2H])([2H])N1C(=O)c2cccc(OC(F)F)c2[C@H]2C[C@@H]1c1nc3ccc(C#CC4CCNCC4)cc3n12 ; [2H]C([2H])([2H])N1C(=O)c2cccc(OC(F)F)c2[C@H]2C[C@@H]1c1nc3ccc(C#CC4CCN(C)CC4)cc3n12]

To a stirred solution of (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (22 mg, 0.047 mmol) in MeOH (1 mL) were added formaldehyde (2 mg, 0.071 mmol) and AcOH (9 mg, 0.141 mmol) in portions at room temperature. The resulting mixture was stirred for 20 min at room temperature. To the above mixture was added NaBH₃CN (4 mg, 0.071 mmol) in portions over 5 min at 0° C. The resulting mixture was stirred for additional 2 h at room temperature. The resulting solution was purified by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min; Gradient: 20% B to 40% B in 10 min; Wave Length: 254 nm/220 nm; RT1(min): 10.93) to afford (7R,14R)-1-(difluoromethoxy)-6-(methyl-d3)-11-((1-methylpiperidin-4-yl)ethynyl)-6,7-dihydro-7,14-methanobenzo[f]benzo[4,5]imidazo[1,2-a][1,4]diazocin-5(14H)-one (7 mg, 31%) as a white solid. MS ESI calculated for C₂₇H₂₃D₃F₂N₄O₂ [M+H]⁺, 480.22 found 480.25. ¹H NMR (400 MHz, Methanol-d₄) δ 8.43-8.31 (m, 1H), 7.60-7.04 (m, 5H), 6.34 (d, J=7.1 Hz, 1H), 5.17 (d, J=7.1 Hz, 1H), 3.59-3.47 (m, 1H), 3.31-2.27 (m, 2H), 2.87 (d, J=13.8 Hz, 1H), 2.81-2.56 (m, 3H), 2.38-2.16 (m, 4H), 2.02-1.89 (m, 2H), 1.80-1.70 (m, 2H). ¹⁹F NMR (377 MHz, Methanol-d₄) δ −82.97, −83.42, −83.59, −84.10.

### Example 58: (7R,14R)-1-(difluoromethoxy)-6-(methyl-d3)-11-(((R)-pyrrolidin-2-yl)ethynyl)-6,7-dihydro-7,14-methanobenzo[f]benzo[4,5]imidazo[1,2-a][1,4]diazocin-5(14H)-one

### Synthetic Scheme

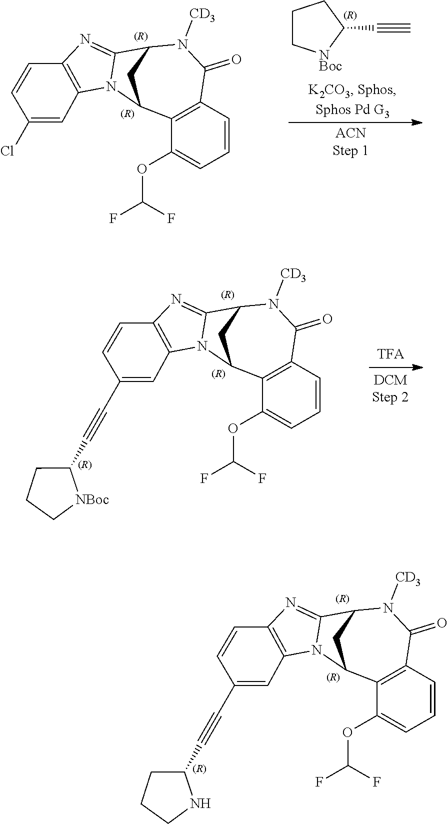

### Preparation 58A: tert-butyl (R)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate

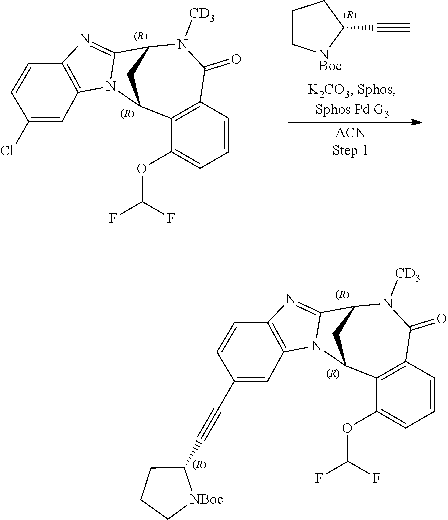

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), tert-butyl (R)-2-ethynylpyrrolidine-1-carboxylate (298 mg, 1.530 mmol), K₂CO₃ (105 mg, 0.765 mmol), SPhos Pd Gen.3 (48 mg, 0.061 mmol) and SPhos (25 mg, 0.061 mmol) in ACN (1.5 mL) was stirred for overnight at 80° C. under nitrogen atmosphere. The mixture was allowed to cool down to room temperature. The residue was purified by silica gel column chromatography, eluted with CH₂Cl₂/MeOH (6/1) to afford tert-butyl (R)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (107 mg, crude) as yellow solid. MS ESI calculated for C₃₀H₂₇D₃F₂N₄O₄ [M+H]⁺, 552.24 found 552.40.

### Example 58: (7R,14R)-1-(difluoromethoxy)-6-(methyl-d3)-11-(((R)-pyrrolidin-2-yl)ethynyl)-6,7-dihydro-7,14-methanobenzo[f]benzo[4,5]imidazo[1,2-a][1,4]diazocin-5(14H)-one

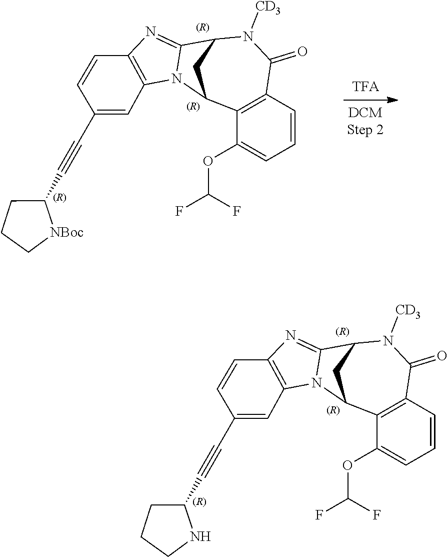

A solution of tert-butyl (R)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (107 mg, 0.194 mmol) in DCM (5 mL) was added TFA (1 mL) and stirred for 2 h at room temperature. The resulting mixture was concentrated under reduced pressure. The residue was purified by reversed-phase flash chromatography with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 15% B to 33% B in 10 min; Wave Length: 254 nm/220 nm; RT1(min): 12.38) to afford (7R,14R)-1-(difluoromethoxy)-6-(methyl-d3)-11-(((R)-pyrrolidin-2-yl)ethynyl)-6,7-dihydro-7,14-methanobenzo[f]benzo[4,5]imidazo[1,2-a][1,4]diazocin-5(14H)-one (3 mg, 3%) as a white solid. MS ESI calculated for C₂₅H₁₉D₃F₂N₄O₂ [M+H]⁺, 452.19 found 452.20. ¹H NMR (400 MHz, Methanol-d₄) δ 8.44-8.30 (m, 1H), 7.59-7.54 (m, 2H), 7.51-7.40 (m, 2H), 7.32-7.03 (m, 2H), 6.35 (d, J=7.2 Hz, 1H), 5.17 (d, J=7.1 Hz, 1H), 4.07-3.94 (m, 1H), 3.62-3.47 (m, 1H), 3.14-3.01 (m, 1H), 2.92-2.83 (m, 2H), 2.21-2.09 (m, 1H), 2.00-1.74 (m, 3H). ¹⁹F NMR (377 MHz, Methanol-d₄) δ −82.98, −83.43, −83.50, −83.95.

### Example 59: (7R,14R)-1-(difluoromethoxy)-6-(methyl-d3)-11-(((S)-pyrrolidin-2-yl)ethynyl)-6,7-dihydro-7,14-methanobenzo[f]benzo[4,5]imidazo[1,2-a][1,4]diazocin-5(14H)-one

### Synthetic Scheme

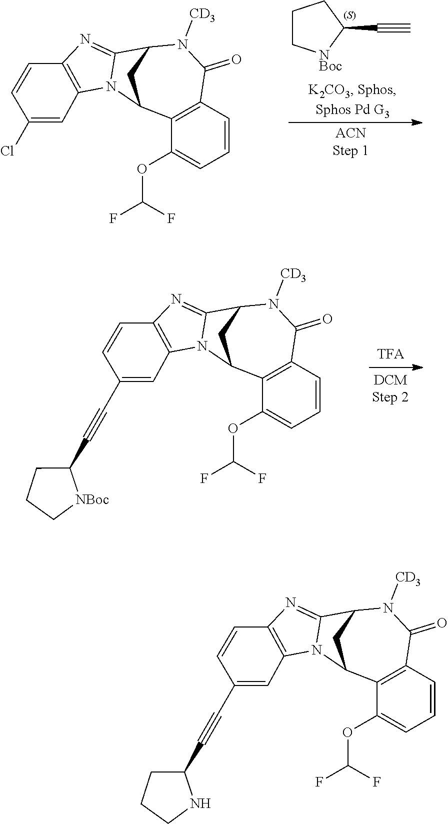

### Preparation 59A: tert-butyl (S)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate

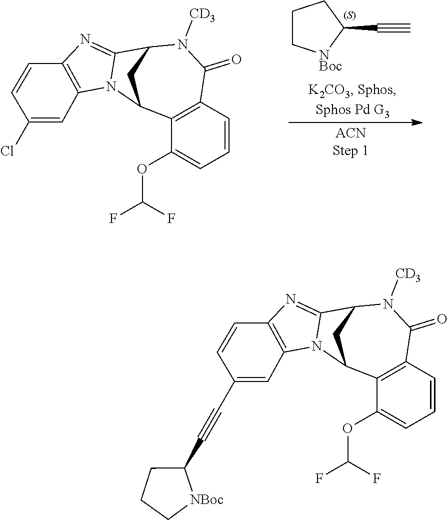

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol), tert-butyl (S)-2-ethynylpyrrolidine-1-carboxylate (199 mg, 1.020 mmol), K₂CO₃ (141 mg, 1.020 mmol), SPhos Pd Gen.3 (32 mg, 0.041 mmol) and SPhos (17 mg, 0.041 mmol) in ACN (1 mL) was stirred for overnight at 80° C. under nitrogen atmosphere. The resulting mixture was concentrated under reduced pressure. The residue was purified by silica gel column chromatography, eluted with CH₂Cl₂/MeOH (9/1) to afford tert-butyl (S)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (95 mg, 84%) as a yellow solid. MS ESI calculated for C₃₀H₂₇D₃F₂N₄O₄[M+H]⁺, 552.24 found 552.40.

### Example 59: (7R,14R)-1-(difluoromethoxy)-6-(methyl-d3)-11-(((S)-pyrrolidin-2-yl)ethynyl)-6,7-dihydro-7,14-methanobenzo[f]benzo[4,5]imidazo[1,2-a][1,4]diazocin-5(14H)-one

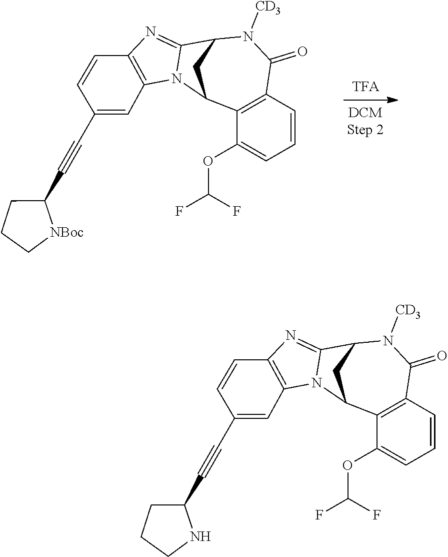 [SMILES: [2H]C([2H])([2H])N1C(=O)c2cccc(OC(F)F)c2[C@H]2C[C@@H]1c1nc3ccc(C#C[C@@H]4CCCN4C(=O)OC(C)(C)C)cc3n12 ; [2H]C([2H])([2H])N1C(=O)c2cccc(OC(F)F)c2[C@H]2C[C@@H]1c1nc3ccc(C#C[C@@H]4CCCN4)cc3n12]

A solution of tert-butyl (S)-2-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (90 mg, 0.163 mmol) in DCM (5 mL) was added TFA (1 mL) and stirred for 2 h at room temperature. The resulting mixture was concentrated under reduced pressure. The resulting solution was purified by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min mL/min; Gradient: 15% B to 33% B in 10 min; Wave Length: 254 nm/220 nm; RT1(min): 11.8) to afford (7R,14R)-1-(difluoromethoxy)-6-(methyl-d3)-11-(((S)-pyrrolidin-2-yl)ethynyl)-6,7-dihydro-7,14-methanobenzo[f]benzo[4,5]imidazo[1,2-a][1,4]diazocin-5(14H)-one (21 mg, 27%) as an orange solid. MS ESI calculated for C₂₅H₁₉D₃F₂N₄O₂ [M+H]⁺, 452.19 found 452.30. ¹H NMR (400 MHz, Methanol-d₄) δ 8.41-8.32 (m, 1H), 7.60-7.53 (m, 2H), 7.51-7.39 (m, 2H), 7.30-7.02 (m, 2H), 6.35 (d, J=7.2 Hz, 1H), 5.17 (d, J=7.1 Hz, 1H), 4.02 (t, J=6.6 Hz, 1H), 3.59-3.47 (m, 1H), 3.13-3.04 (m, 1H), 2.95-2.82 (m, 2H), 2.21-2.10 (m, 1H), 2.01-1.74 (m, 3H). ¹⁹F NMR (377 MHz, Methanol-d₄) δ −82.95, −83.41, −83.49, −83.94.

### Example 60: (7R,14R)-1-(difluoromethoxy)-6-(methyl-d3)-11-(piperidin-3-ylethynyl)-6,7-dihydro-7,14-methanobenzo[f]benzo[4,5]imidazo[1,2-a][1,4]diazocin-5(14H)-one

### Synthetic Scheme

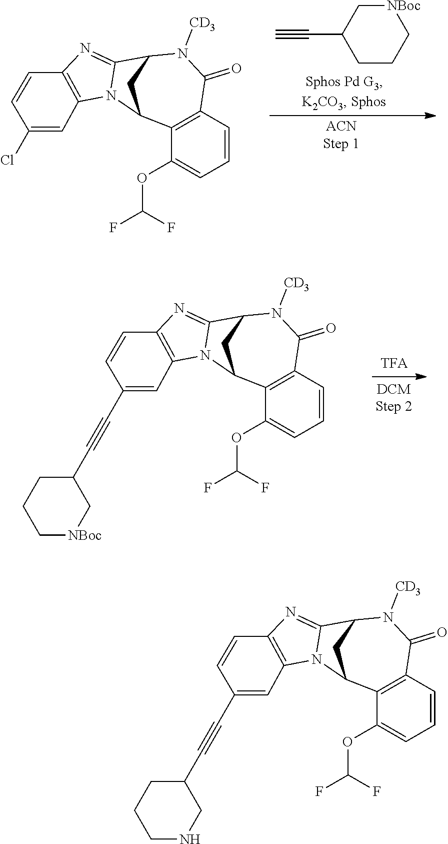 [SMILES: [2H]C([2H])([2H])N1C(=O)c2cccc(OC(F)F)c2[C@H]2C[C@@H]1c1nc3ccc(Cl)cc3n12 ; C#CC1CCCN(C(=O)OC(C)(C)C)C1 ; [2H]C([2H])([2H])N1C(=O)c2cccc(OC(F)F)c2[C@H]2C[C@@H]1c1nc3ccc(C#CC4CCCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CCCNC4)cc3n12]

### Preparation 60A: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate

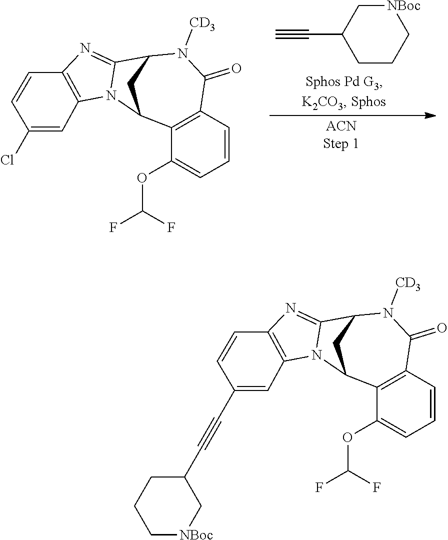 [SMILES: [2H]C([2H])([2H])N1C(=O)c2cccc(OC(F)F)c2[C@H]2C[C@@H]1c1nc3ccc(Cl)cc3n12 ; C#CC1CCCN(C(=O)OC(C)(C)C)C1 ; [2H]C([2H])([2H])N1C(=O)c2cccc(OC(F)F)c2[C@H]2C[C@@H]1c1nc3ccc(C#CC4CCCN(C(=O)OC(C)(C)C)C4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and tert-butyl 3-ethynylpiperidine-1-carboxylate (213 mg, 1.020 mmol) in ACN (1 mL) were added K₂CO₃ (141 mg, 1.020 mmol), SPhos (16 mg, 0.041 mmol) and SPhos Pd Gen.3 (31 mg, 0.041 mmol) in portions at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (0% to 10%) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (110 mg, crude) as yellow oil. MS ESI calculated for C₃₁H₂₉D₃F₂N₄O₄[M+H]⁺, 566.26. found 566.35.

### Example 60: (7R,14R)-1-(difluoromethoxy)-6-(methyl-d3)-11-(piperidin-3-ylethynyl)-6,7-dihydro-7,14-methanobenzo[f]benzo[4,5]imidazo[1,2-a][1,4]diazocin-5(14H)-one

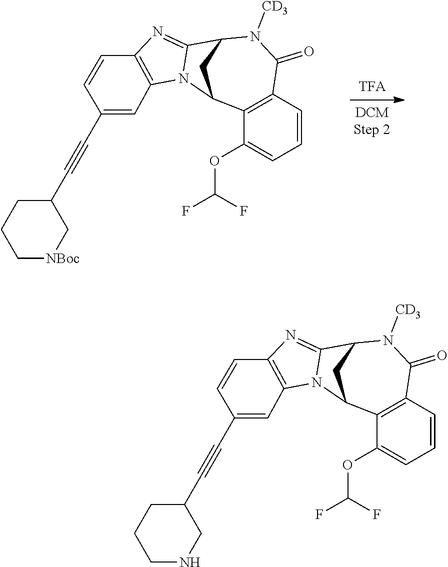 [SMILES: [2H]C([2H])([2H])N1C(=O)c2cccc(OC(F)F)c2[C@H]2C[C@@H]1c1nc3ccc(C#CC4CCCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CCCNC4)cc3n12]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (110 mg, 0.194 mmol) in DCM (5 mL) was added TFA (1 mL) dropwise at 0° C. The resulting mixture was stirred for 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 20% to 50% gradient in 20 min; detector, 254/220 nm to afford (7R,14R)-1-(difluoromethoxy)-6-(methyl-d3)-11-(piperidin-3-ylethynyl)-6,7-dihydro-7,14-methanobenzo[f]benzo[4,5]imidazo[1,2-a][1,4]diazocin-5(14H)-one (33 mg, 36%) as a white solid. MS ESI calculated for C₂₆H₂₁D₃F₂N₄O₂ [M+H]⁺, 466.21. found 466.10. ¹H NMR (400 MHz, Chloroform-d) δ 8.53-8.42 (m, 1H), 7.61 (d, J=8.5 Hz, 1H), 7.50 (d, J=2.6 Hz, 1H), 7.44-7.28 (m, 3H), 6.90 (t, J=72.7 Hz, 1H), 6.17 (d, J=7.2 Hz, 1H), 4.92 (d, J=7.1 Hz, 1H), 3.48-3.35 (m, 2H), 3.28-3.16 (m, 1H), 3.10-3.01 (m, 1H), 2.97-2.74 (m, 3H), 2.16-1.65 (m, 4H). ¹⁹F NMR (377 MHz, Chloroform-d) δ −80.00, −80.12, −80.45, −80.57, −81.42, −81.47, −81.87, −81.91.

### Example 61: (7R,14R)-1-(difluoromethoxy)-11-(3-(dimethylamino)prop-1-yn-1-yl)-6-(methyl-d3)-6,7-dihydro-7,14-methanobenzo[f]benzo[4,5]imidazo[1,2-a][1,4]diazocin-5(14H)-one

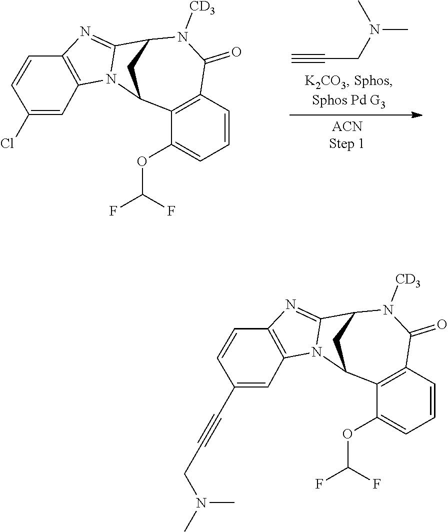

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), N,N-dimethylprop-2-yn-1-amine (63 mg, 0.765 mmol), K₂CO₃ (105 mg, 0.765 mmol), SPhos Pd Gen.3 (24 mg, 0.031 mmol) and SPhos (13 mg, 0.031 mmol) in ACN (1 mL) was stirred for 2 h at 80° C. under nitrogen atmosphere. The resulting mixture was concentrated under reduced pressure. The residue was purified by silica gel column chromatography, eluted with CH₂Cl₂/MeOH (8/1) followed by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 20% B to 38% B in 10 min; Wave Length: 254 nm/220 nm; RT1(min): 11.48) to afford (7R,14R)-1-(difluoromethoxy)-11-(3-(dimethylamino)prop-1-yn-1-yl)-6-(methyl-d3)-6,7-dihydro-7,14-methanobenzo[f]benzo[4,5]imidazo[1,2-a][1,4]diazocin-5(14H)-one (30 mg, 44%) as white solid. MS ESI calculated for C₂₄H₁₉D₃F₂N₄O₂ [M+H]⁺, 440.19. found 440.30. ¹H NMR (400 MHz, Chloroform-d) δ 8.55-8.42 (m, 1H), 7.63 (d, J=8.4 Hz, 1H), 7.56 (d, J=1.6 Hz, 1H), 7.42 (t, J=8.2 Hz, 1H), 7.36-7.30 (m, 2H), 6.85 (t, J=72.8 Hz, 1H), 6.21 (d, J=7.2 Hz, 1H), 4.93 (d, J=7.1 Hz, 1H), 3.55 (d, J=3.8 Hz, 2H), 3.51-3.36 (m, 1H), 2.86 (d, J=13.5 Hz, 1H), 2.44 (s, 6H). ¹⁹F NMR (377 MHz, Chloroform-d) δ −80.07, −80.51, −81.10, −81.54.

### Example 62: (7R,14R)-11-(3-(diethylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

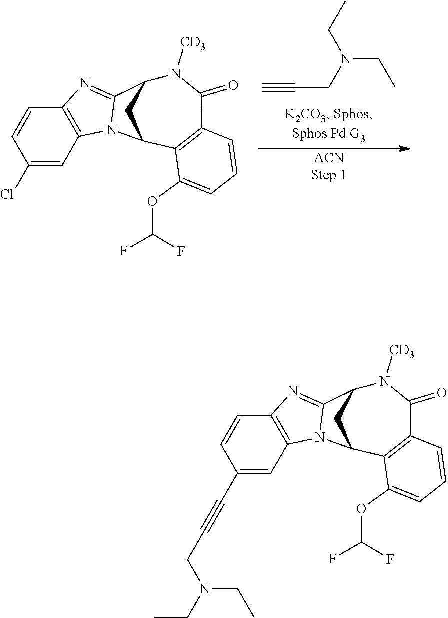

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), N,N-diethylprop-2-yn-1-amine (85 mg, 0.765 mmol), K₂CO₃ (106 mg, 0.765 mmol), SPhos Pd Gen.3 (24 mg, 0.031 mmol) and SPhos (13 mg, 0.031 mmol) in ACN (1 mL) was stirred for 2 h at 80° C. under nitrogen atmosphere. The resulting mixture was concentrated under reduced pressure. The residue was purified by silica gel column chromatography, eluted with CH₂Cl₂/MeOH (9/1) followed by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 26% B to 44% B in 10 min; Wave Length: 254 nm/220 nm; RT1(min): 11.13) to afford (7R,14R)-11-(3-(diethylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (16 mg, 22%) as white solid. MS ESI calculated for C₂₆H₂₃D₃F₂N₄O₂ [M+H]⁺, 468.22 found 468.35. ¹H NMR (400 MHz, Chloroform-d) δ 8.51-8.43 (m, 1H), 7.62 (d, J=8.4 Hz, 1H), 7.55 (d, J=1.6 Hz, 1H), 7.42 (t, J=8.2 Hz, 1H), 7.35-7.28 (m, 2H), 6.84 (t, J=72.8 Hz, 1H), 6.21 (d, J=7.2 Hz, 1H), 4.93 (d, J=7.1 Hz, 1H), 3.72 (s, 2H), 3.52-3.37 (m, 1H), 2.86 (d, J=13.6 Hz, 1H), 2.78-2.66 (m, 4H), 1.18 (t, J=7.1 Hz, 6H). ¹⁹F NMR (377 MHz, Chloroform-d) δ −80.14, −80.58, −80.96, −81.41.

### Example 63: (7R,14R)-1-(difluoromethoxy)-6-(methyl-d3)-11-((1-methylpiperidin-3-yl)ethynyl)-6,7-dihydro-7,14-methanobenzo[f]benzo[4,5]imidazo[1,2-a][1,4]diazocin-5(14H)-one

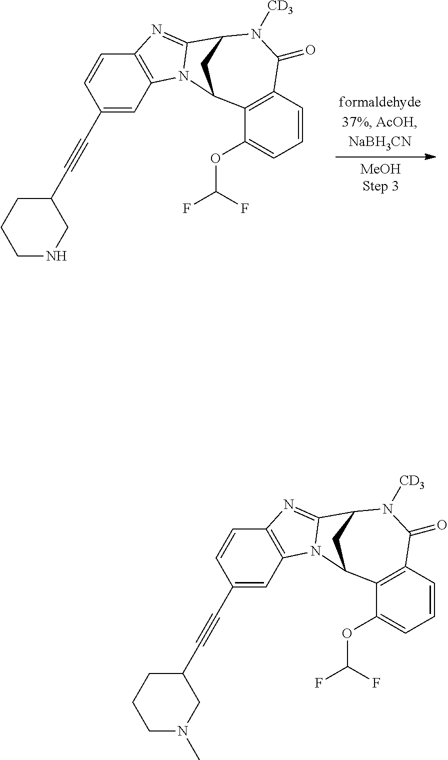 [SMILES: [2H]C([2H])([2H])N1C(=O)c2cccc(OC(F)F)c2[C@H]2C[C@@H]1c1nc3ccc(C#CC4CCCNC4)cc3n12 ; [2H]C([2H])([2H])N1C(=O)c2cccc(OC(F)F)c2[C@H]2C[C@@H]1c1nc3ccc(C#CC4CCCN(C)C4)cc3n12]

To a stirred solution of (7R,14R)-1-(difluoromethoxy)-6-(methyl-d3)-11-(piperidin-3-ylethynyl)-6,7-dihydro-7,14-methanobenzo[f]benzo[4,5]imidazo[1,2-a][1,4]diazocin-5(14H)-one (23 mg, 0.049 mmol) in MeOH (0.5 mL) were added AcOH (9 mg, 0.147 mmol) and formaldehyde (6 mg, 0.074 mmol, 37%) in portions at room temperature. The resulting mixture was stirred for 30 min at room temperature. To the above mixture was added NaBH₃CN (5 mg, 0.074 mmol) dropwise at 0° C. The resulting mixture was stirred for additional 2 h at room temperature. The resulting mixture was concentrated under vacuum. The residue was purified by reversed-phase flash chromatography with the following conditions: column, C18 silica gel; mobile phase, CH₃CN in water (10 mmol/L NH₄HCO₃), 30% to 50% gradient in 20 min; detector, 254/220 nm to afford (7R,14R)-1-(difluoromethoxy)-6-(methyl-d3)-11-((1-methylpiperidin-3-yl)ethynyl)-6,7-dihydro-7,14-methanobenzo[f]benzo[4,5]imidazo[1,2-a][1,4]diazocin-5(14H)-one (6 mg, 23%) as a white solid. MS ESI calculated for C₂₇H₂₃D₃F₂N₄O₂[M+H]⁺, 480.22. found 480.25. ¹H NMR (400 MHz, Methanol-d₄) δ 8.37 (d, J=7.7 Hz, 1H), 7.54 (d, J=8.9 Hz, 2H), 7.50-7.41 (m, 2H), 7.27-7.04 (m, 2H), 6.34 (d, J=7.4 Hz, 1H), 5.17 (d, J=7.5 Hz, 1H), 3.58-3.46 (m, 1H), 3.00-2.70 (m, 4H), 2.30 (s, 3H), 2.20-1.95 (m, 3H), 1.85-1.72 (m, 1H), 1.69-1.54 (m, 1H), 1.50-1.37 (m, 1H). ¹⁹F NMR (377 MHz, Methanol-d₄) δ −83.58, −83.61.

### Example 64: (7R,14R)-11-(3-amino-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

### Synthetic Scheme

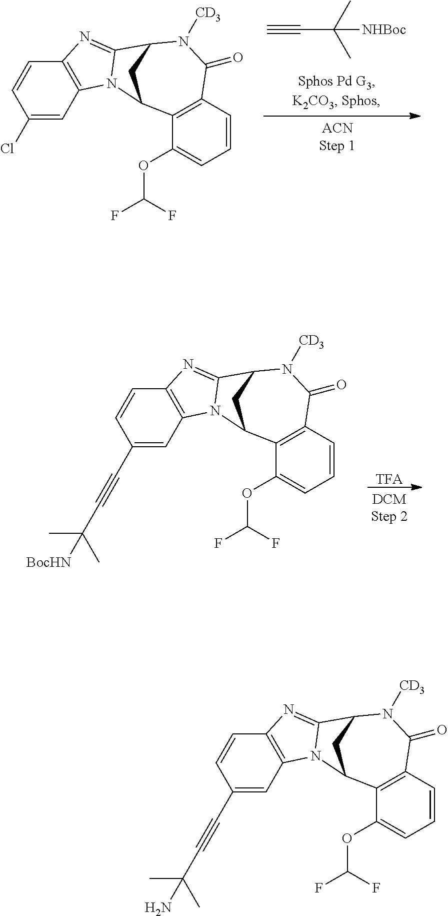

### Preparation 64A: tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-2-yl)carbamate

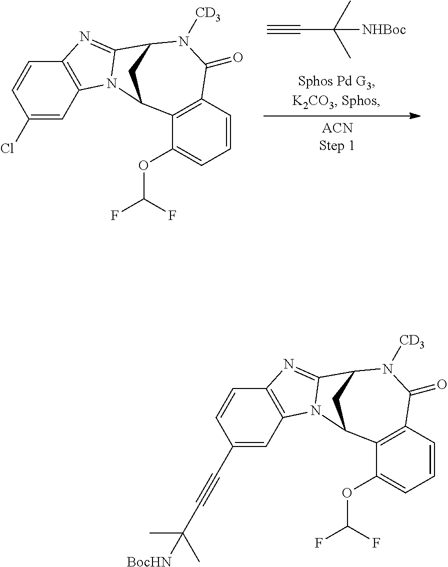

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and tert-butyl (2-methylbut-3-yn-2-yl)carbamate (140 mg, 0.765 mmol) in ACN (1 mL) were added K₂CO₃ (106 mg, 0.765 mmol) SPhos Pd Gen.3 (24 mg, 0.031 mmol) and SPhos (13 mg, 0.031 mmol) in portions at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (9/1) to afford tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-2-yl)carbamate (70 mg, crude) as yellow solid. MS ESI calculated for C₂₉H₂₇D₃F₂N₄O₄[M+H]⁺, 540.24 found 540.40.

### Example 64: (7R,14R)-11-(3-amino-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

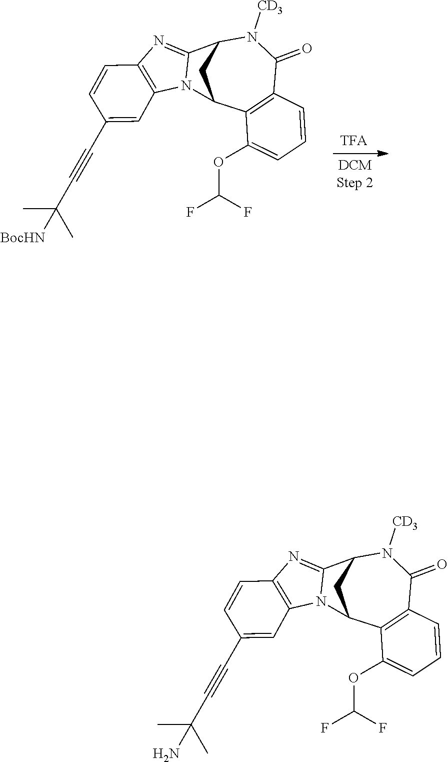 [SMILES: [2H]C([2H])([2H])N1C(=O)c2cccc(OC(F)F)c2[C@H]2C[C@@H]1c1nc3ccc(C#CC(C)(C)NC(=O)OC(C)(C)C)cc3n12 ; [2H]C([2H])([2H])N1C(=O)c2cccc(OC(F)F)c2[C@H]2C[C@@H]1c1nc3ccc(C#CC(C)(C)N)cc3n12]

A solution of tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-2-yl)carbamate (70 mg, 0.130 mmol) in DCM (5 mL) was added TFA (1 mL) and stirred for 2 h at room temperature. The resulting mixture was concentrated under reduced pressure. The resulting solution was purified by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 17% B to 33% B in 10 min; Wave Length: 254 nm/220 nm; RT1(min): 14.08) to afford (7R,14R)-11-(3-amino-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 34%) as white solid. MS ESI calculated for C₂₄H₁₉D₃F₂N₄O₂ [M+H]⁺, 440.19 found 440.35. ¹H NMR (400 MHz, Chloroform-d) δ 8.51-8.45 (m, 1H), 7.61 (d, J=8.4 Hz, 1H), 7.51-7.46 (m, 1H), 7.42 (t, J=8.2 Hz, 1H), 7.34 (d, J=8.1 Hz, 1H), 7.25 (d, J=1.6 Hz, 1H), 6.85 (t, J=73.3 Hz, 1H), 6.20 (d, J=7.2 Hz, 1H), 4.94 (d, J=7.2 Hz, 1H), 3.53-3.40 (m, 1H), 2.85 (d, J=13.5 Hz, 1H), 1.50 (s, 6H). ¹⁹F NMR (377 MHz, Chloroform-d) δ −79.96, −80.40, −81.24, −81.68.

### Example 65: (7R,14R)-1-(difluoromethoxy)-11-(3-(ethylamino)prop-1-yn-1-yl)-6-(methyl-d3)-6,7-dihydro-7,14-methanobenzo[f]benzo[4,5]imidazo[1,2-a][1,4]diazocin-5(14H)-one

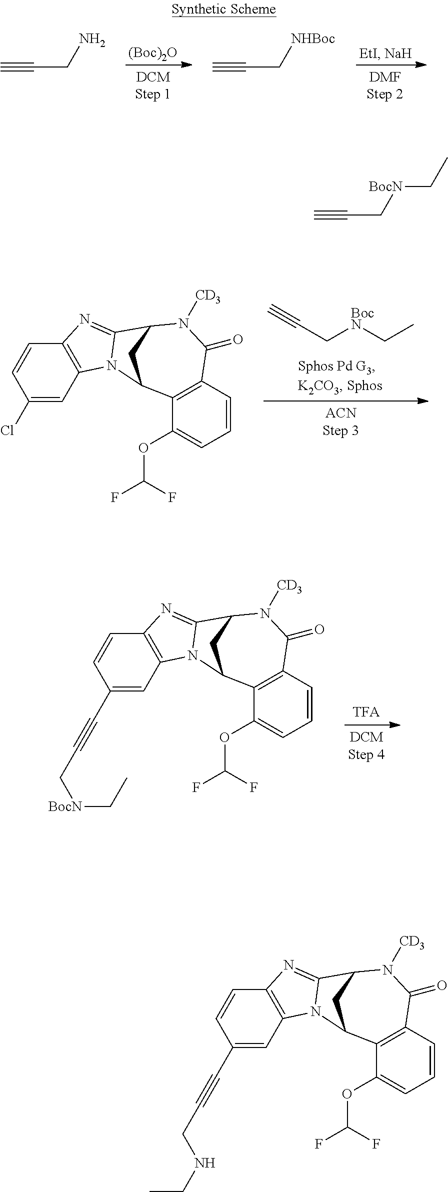

### Preparation 65A: tert-butyl N-(prop-2-yn-1-yl)carbamate

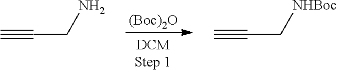

A solution of 2-propynylamine (4.00 g, 72.622 mmol) and (Boc)₂O (15.85 g, 72.622 mmol) in DCM (120 mL) was stirred for 3 h at room temperature under nitrogen atmosphere. The resulting mixture was concentrated under vacuum to afford tert-butyl N-(prop-2-yn-1-yl)carbamate (11.00 g, 97%) as a white solid. ¹H NMR (400 MHz, Chloroform-d) δ 4.71 (s, 1H), 3.92 (s, 2H), 2.22 (t, J=2.5 Hz, 1H), 1.46 (s, 9H).

### Preparation 65B: tert-butyl N-ethyl-N-(prop-2-yn-1-yl)carbamate

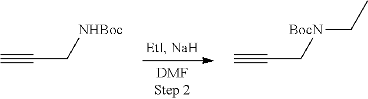

To a solution of tert-butyl N-(prop-2-yn-1-yl)carbamate (2.00 g, 12.887 mmol) in DMF (20 mL) was added NaH (0.62 g, 15.464 mmol, 60%) at 0 degrees C. The mixture was stirred for 15 min. Ethyl iodide (2.41 g, 15.464 mmol) was added and the mixture was allowed to warm to RT and stirred for 3 h. The reaction mixture was quenched by water and extracted with EtOAc (3×25 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/PE (1/1) to afford tert-butyl N-ethyl-N-(prop-2-yn-1-yl)carbamate (1.20 g, 50%) as a colorless oil. ¹H NMR (400 MHz, Chloroform-d) δ 4.04 (s, 2H), 3.37 (q, J=7.2 Hz, 2H), 2.18 (t, J=2.4 Hz, 1H), 1.47 (s, 9H), 1.14 (t, J=7.2 Hz, 3H).

### Preparation 65C: tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(ethyl)carbamate

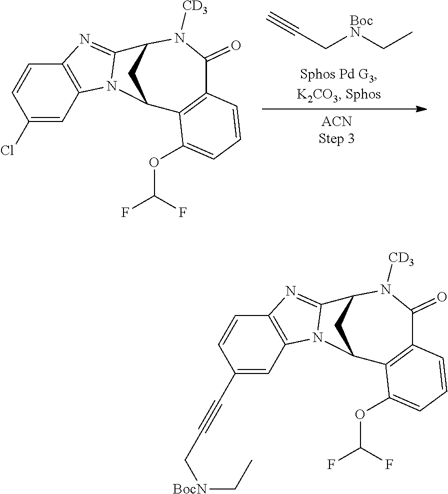

Into a 20 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (120 mg, 0.305 mmol), tert-butyl N-ethyl-N-(prop-2-yn-1-yl)carbamate (280 mg, 1.525 mmol), SPhos (25 mg, 0.061 mmol), SPhos Pd Gen.3 (23 mg, 0.030 mmol), K₂CO₃ (211.10 mg, 1.525 mmol) and ACN (4 mL) at room temperature. The mixture was purged with nitrogen for 5 min and then was stirred for 16 h at 80° C. under nitrogen atmosphere. The resulting mixture was concentrated under reduced pressure. The residue was purified by silica gel column chromatography, eluted with DCM/EA (1/10) to afford tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(ethyl)carbamate (160 mg, crude) as a brown solid. MS ESI calculated for C₂₉H₂₇D₃F₂N₄O₄[M+H]⁺, 540.24. found 540.10.

### Example 65: (7R,14R)-1-(difluoromethoxy)-11-(3-(ethylamino)prop-1-yn-1-yl)-6-(methyl-d3)-6,7-dihydro-7,14-methanobenzo[f]benzo[4,5]imidazo[1,2-a][1,4]diazocin-5(14H)-one

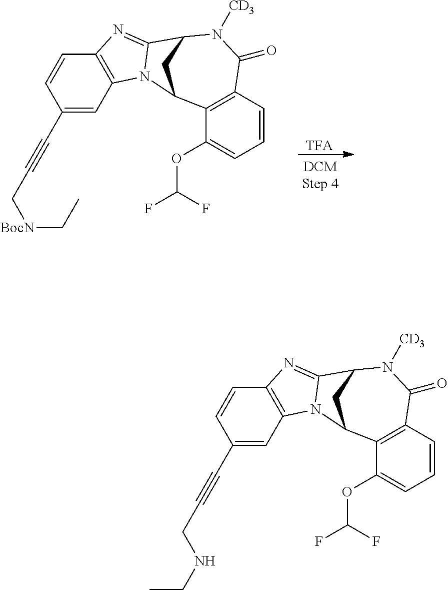 [SMILES: [2H]C([2H])([2H])N1C(=O)c2cccc(OC(F)F)c2[C@H]2C[C@@H]1c1nc3ccc(C#CCN(CC)C(=O)OC(C)(C)C)cc3n12 ; [2H]C([2H])([2H])N1C(=O)c2cccc(OC(F)F)c2[C@H]2C[C@@H]1c1nc3ccc(C#CCNCC)cc3n12 ; "TFA"]

To a stirred solution of tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(ethyl)carbamate (160 mg, 0.297 mmol) in DCM (5 mL) was added TFA (2 mL) dropwise at room temperature under nitrogen atmosphere. The resulting mixture was stirred for 2 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 5% to 50% gradient in 20 min; detector, 254 nm to afford (7R,14R)-1-(difluoromethoxy)-11-(3-(ethylamino)prop-1-yn-1-yl)-6-(methyl-d3)-6,7-dihydro-7,14-methanobenzo[f]benzo[4,5]imidazo[1,2-a][1,4]diazocin-5(14H)-one (60 mg, 44%) as a tan solid. MS ESI calculated for C₂₄H₁₉D₃F₂N₄O₂ [M+H]⁺, 440.19. found 440.25. ¹H NMR (400 MHz, Chloroform-d) δ 8.51-8.43 (m, 1H), 7.65-7.58 (m, 1H), 7.52 (d, J=1.2 Hz, 1H), 7.42 (t, J=8.0 Hz, 1H), 7.36-7.27 (m, 2H), 6.83 (t, J=73.2 Hz, 1H), 6.20 (d, J=7.2 Hz, 1H), 4.93 (d, J=7.2 Hz, 1H), 3.67 (s, 2H), 3.50-3.35 (m, 1H), 2.91-2.75 (m, 3H), 1.17 (t, J=7.2 Hz, 3H). ¹⁹F NMR (376 MHz, Chloroform-d) δ −79.94, −80.38, −81.08, −81.53.

### Example 66: (7R,14R)-1-(difluoromethoxy)-11-(3-(isopropylamino)prop-1-yn-1-yl)-6-(methyl-d3)-6,7-dihydro-7,14-methanobenzo[f]benzo[4,5]imidazo[1,2-a][1,4]diazocin-5(14H)-one

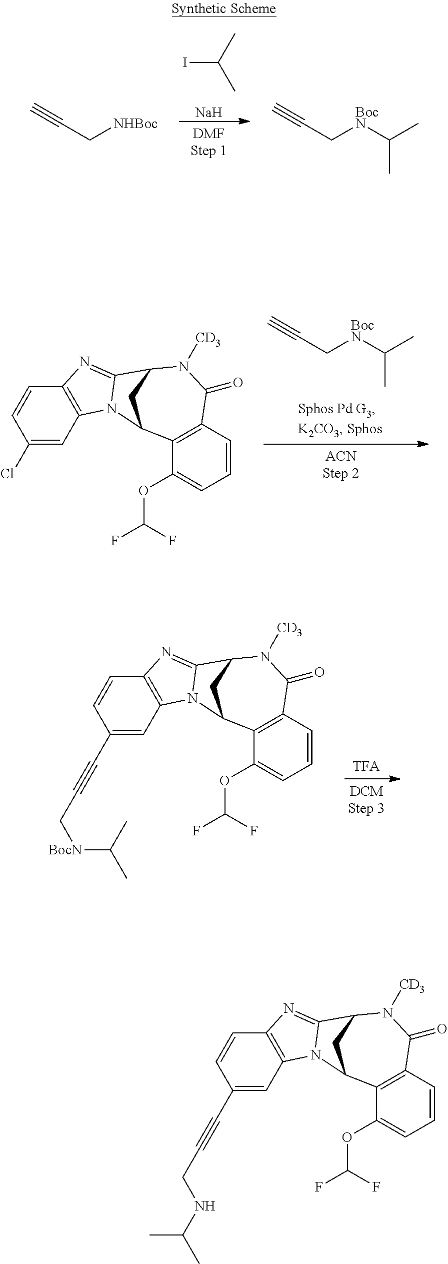

### Preparation 66A: Tert-Butyl isopropyl(prop-2-yn-1-yl)carbamate

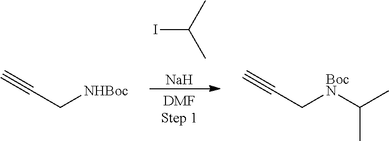

To a solution of tert-butyl prop-2-yn-1-ylcarbamate (2.00 g, 12.887 mmol) in DMF (20 mL) was added NaH (0.62 g, 15.464 mmol, 60%) in portions at 0° C. The mixture was stirred under nitrogen atmosphere for 15 min. Then 2-iodopropane (2.63 g, 15.464 mmol) was added and the mixture was allowed to warm to RT and stirred for overnight. The reaction mixture was quenched with water and extracted with EtOAc (3×25 mL). The combined organic layers were washed with brine (50 mL), dried over anhydrous Na₂SO₄. After filtration, the filtrate was concentrated under reduced pressure. The residue was purified by silica gel column chromatography, eluted with CH₂Cl₂/PE (1/1) to afford tert-butyl isopropyl(prop-2-yn-1-yl)carbamate (180 mg, 7%) as a colorless oil. ¹H NMR (400 MHz, Chloroform-d) δ 4.44-4.07 (m, 1H), 3.89 (s, 2H), 2.13 (t, J=2.4 Hz, 1H), 1.46 (s, 9H), 1.20 (d, J=6.8 Hz, 6H).

### Preparation 66B: tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(isopropyl)carbamate

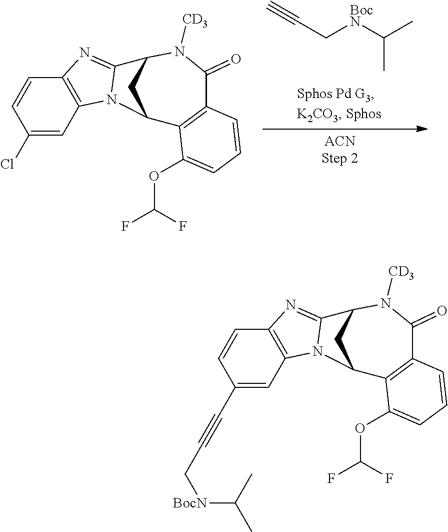 [SMILES: [2H]C([2H])([2H])N1C(=O)c2cccc(OC(F)F)c2[C@H]2C[C@@H]1c1nc3ccc(Cl)cc3n12 ; C#CCN(C(=O)OC(C)(C)C)C(C)C ; [2H]C([2H])([2H])N1C(=O)c2cccc(OC(F)F)c2[C@H]2C[C@@H]1c1nc3ccc(C#CCN(C(=O)OC(C)(C)C)C(C)C)cc3n12]

Into a 8 mL vial were added (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol), tert-butyl isopropyl(prop-2-yn-1-yl)carbamate (175 mg, 0.890 mmol), SPhos (15 mg, 0.036 mmol), SPhos Pd Gen.3 (14 mg, 0.018 mmol), K₂CO₃ (123 mg, 0.890 mmol) and ACN (3 mL) at room temperature. The mixture was purged with nitrogen for 5 min and then was stirred for 16 h at 80° C. under nitrogen atmosphere. The resulting mixture was concentrated under reduced pressure. The residue was purified by silica gel column chromatography, eluted with DCM/EA (1/10) to afford tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(isopropyl)carbamate (90 mg, crude) as a brown solid. MS ESI calculated for C₃₀H₂₉D₃F₂N₄O₄[M+H]⁺, 554.26. found 554.30.

### Example 66: (7R,14R)-1-(difluoromethoxy)-11-(3-(isopropylamino)prop-1-yn-1-yl)-6-(methyl-d3)-6,7-dihydro-7,14-methanobenzo[f]benzo[4,5]imidazo[1,2-a][1,4]diazocin-5(14H)-one

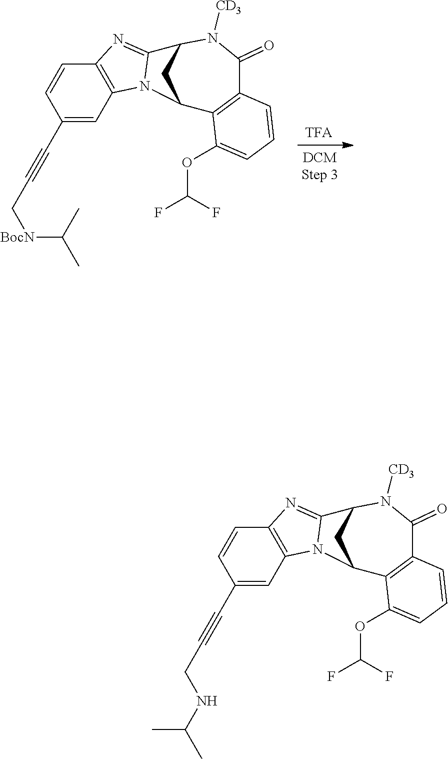 [SMILES: [2H]C([2H])([2H])N1C(=O)c2cccc(OC(F)F)c2[C@H]2C[C@@H]1c1nc3ccc(C#CCN(C(=O)OC(C)(C)C)C(C)C)cc3n12 ; [2H]C([2H])([2H])N1C(=O)c2cccc(OC(F)F)c2[C@H]2C[C@@H]1c1nc3ccc(C#CCNC(C)C)cc3n12]

To a stirred solution of tert-butyl (3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)(isopropyl)carbamate (90 mg, 0.163 mmol) in DCM (2 mL) was added TFA (1 mL) dropwise at room temperature under nitrogen atmosphere. The resulting mixture was stirred for 2 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 15% to 60% gradient in 25 min; detector, 254 nm to afford (7R,14R)-1-(difluoromethoxy)-11-(3-(isopropylamino)prop-1-yn-1-yl)-6-(methyl-d3)-6,7-dihydro-7,14-methanobenzo[f]benzo[4,5]imidazo[1,2-a][1,4]diazocin-5(14H)-one (15 mg, 19%) as a white solid. MS ESI calculated for C₂₅H₂₁D₃F₂N₄O₂ [M+H]⁺, 454.21. found 454.35. ¹H NMR (400 MHz, Chloroform-d) δ 8.54-8.36 (m, 1H), 7.67-7.56 (m, 1H), 7.51 (s, 1H), 7.46-7.37 (m, 1H), 7.36-7.27 (m, 2H), 6.83 (t, J=73.2 Hz, 1H), 6.29-6.10 (m, 1H), 5.02-4.78 (m, 1H), 3.66 (s, 2H), 3.51-3.34 (m, 1H), 3.19-3.02 (m, 1H), 2.94-2.76 (m, 1H), 1.11 (d, J=4.4 Hz, 6H). ¹⁹F NMR (377 MHz, Chloroform-d) δ −79.97, −80.41, −81.04, −81.49.

### Example 67: (7R,14R)-1-(difluoromethoxy)-11-(3-(ethyl(methyl)amino)prop-1-yn-1-yl)-6-(methyl-d3)-6,7-dihydro-7,14-methanobenzo[f]benzo[4,5]imidazo[1,2-a][1,4]diazocin-5(14H)-one

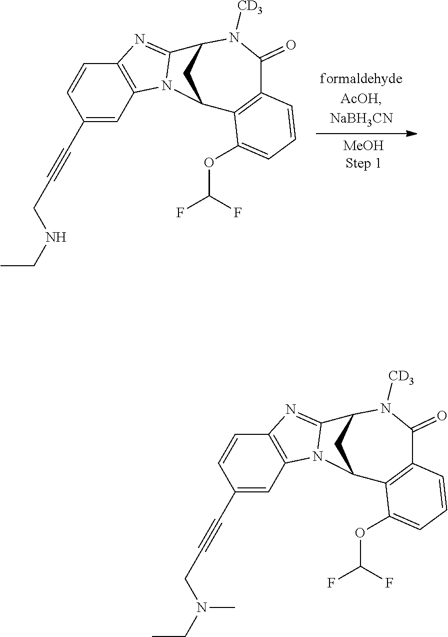 [SMILES: [2H]C([2H])([2H])N1C(=O)c2cccc(OC(F)F)c2[C@H]2C[C@@H]1c1nc3ccc(C#CCNCC)cc3n12 ; [2H]C([2H])([2H])N1C(=O)c2cccc(OC(F)F)c2[C@H]2C[C@@H]1c1nc3ccc(C#CCN(C)CC)cc3n12]

A solution of (7R,14R)-1-(difluoromethoxy)-11-(3-(ethylamino)prop-1-yn-1-yl)-6-(methyl-d3)-6,7-dihydro-7,14-methanobenzo[f]benzo[4,5]imidazo[1,2-a][1,4]diazocin-5(14H)-one (40 mg, 0.091 mmol), AcOH (16 mg, 0.273 mmol) and formaldehyde (14 mg, 0.182 mmol, 37%) in MeOH (3 mL) was stirred for 15 min at room temperature. To the above mixture was added NaBH₃CN (11 mg, 0.182 mmol) in portions at room temperature. The resulting mixture was stirred for additional overnight at room temperature. The resulting mixture was concentrated under vacuum. The residue was purified by silica gel column chromatography, eluted with DCM/MeOH (0% to 10%) followed by Prep-HPLC with the following conditions: column, C18 silica gel; mobile phase, CH₃CN in water (10 mmol/L NH₄HCO₃), 30% to 60% gradient in 25 min; detector, 254 nm to afford (7R,14R)-1-(difluoromethoxy)-11-(3-(ethyl(methyl)amino)prop-1-yn-1-yl)-6-(methyl-d3)-6,7-dihydro-7,14-methanobenzo[f]benzo[4,5]imidazo[1,2-a][1,4]diazocin-5(14H)-one (16 mg, 39%) as a white solid. MS ESI calculated for C₂₅H₂₁D₃F₂N₄O₂ [M+H]⁺, 454.21. found 454.20. ¹H NMR (400 MHz, Chloroform-d) δ 8.51-8.45 (m, 1H), 7.62 (d, J=8.4 Hz, 1H), 7.58-7.53 (m, 1H), 7.42 (t, J=8.2 Hz, 1H), 7.36-7.27 (m, 2H), 6.84 (t, J=73.2 Hz, 1H), 6.21 (d, J=7.2 Hz, 1H), 4.93 (d, J=7.1 Hz, 1H), 3.64 (s, 2H), 3.49-3.38 (m, 1H), 2.86 (d, J=13.6 Hz, 1H), 2.65 (q, J=7.2 Hz, 2H), 2.45 (s, 3H), 1.18 (t, J=7.2 Hz, 3H). ¹⁹F NMR (377 MHz, Chloroform-d) δ −80.10, −80.55, −81.04, −81.49.

### Example 68: (7R,14R)-1-(difluoromethoxy)-6-(methyl-d3)-11-((1-methylazetidin-3-yl)ethynyl)-6,7-dihydro-7,14-methanobenzo[f]benzo[4,5]imidazo[1,2-a][1,4]diazocin-5(14H)-one

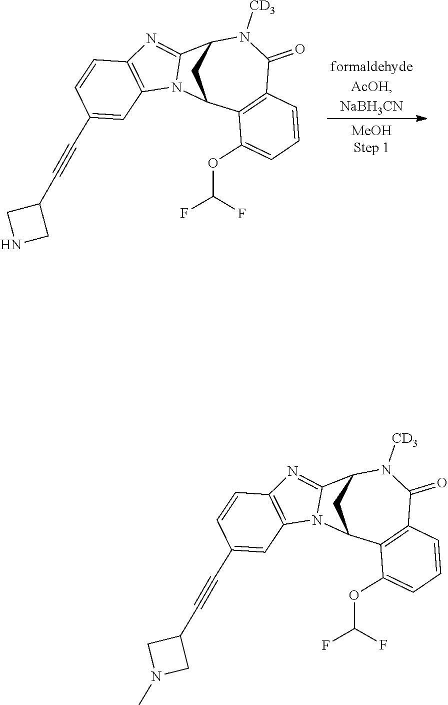 [SMILES: [2H]C([2H])([2H])N1C(=O)c2cccc(OC(F)F)c2[C@H]2C[C@@H]1c1nc3ccc(C#CC4CNC4)cc3n12 ; "formaldehydeAcOH, NaBH3CN" ; [2H]C([2H])([2H])N1C(=O)c2cccc(OC(F)F)c2[C@H]2C[C@@H]1c1nc3ccc(C#CC4CN(C)C4)cc3n12]

A solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 0.046 mmol), formaldehyde (4 mg, 0.055 mmol, 37%) and AcOH (8 uL, 0.138 mmol) in MeOH (1 mL) was stirred for 30 min at room temperature. To the above mixture was added NaBH₃CN (4 mg, 0.069 mmol) at room temperature. The resulting mixture was stirred for additional 2 h at room temperature. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 70% gradient in 20 min; detector, 254 nm. This resulted in (7R,14R)-1-(difluoromethoxy)-6-(methyl-d3)-11-((1-methylazetidin-3-yl)ethynyl)-6,7-dihydro-7,14-methanobenzo[f]benzo[4,5]imidazo[1,2-a][1,4]diazocin-5(14H)-one (11 mg, 52%) as a white solid. MS ESI calculated for C₂₅H₁₉D₃F₂N₄O₂ [M+H]⁺, 452.19. found 452.10. ¹H NMR (400 MHz, Chloroform-d) δ 8.50-8.46 (m, 1H), 7.61 (d, J=8.5 Hz, 1H), 7.51-7.48 (m, 1H), 7.42 (t, J=8.2 Hz, 1H), 7.34 (d, J=8.3 Hz, 1H), 7.29 (d, J=1.5 Hz, 1H), 7.04-6.66 (m, 1H), 6.20 (d, J=7.3 Hz, 1H), 4.93 (d, J=7.0 Hz, 1H), 3.78 (t, J=7.5 Hz, 2H), 3.54-3.38 (m, 2H), 3.23 (t, J=7.6 Hz, 2H), 2.85 (d, J=13.6 Hz, 1H), 2.41 (s, 3H). ¹⁹F NMR (377 MHz, Chloroform-d) δ −79.90, −80.35, −81.13, −81.57.

### Example 69: (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one

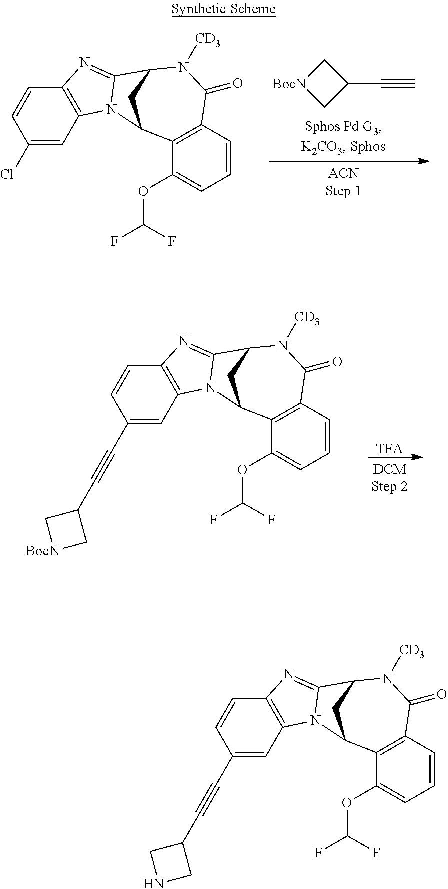 [SMILES: [2H]C([2H])([2H])N1C(=O)c2cccc(OC(F)F)c2[C@H]2C[C@@H]1c1nc3ccc(Cl)cc3n12 ; C#CC1CN(C(=O)OC(C)(C)C)C1 ; "TFA" ; [2H]C([2H])([2H])N1C(=O)c2cccc(OC(F)F)c2[C@H]2C[C@@H]1c1nc3ccc(C#CC4CN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CNC4)cc3n12]

### Preparation 69A: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)azetidine-1-carboxylate

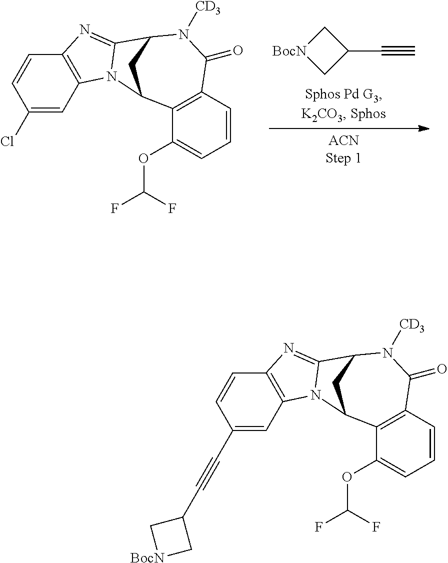 [SMILES: [2H]C([2H])([2H])N1C(=O)c2cccc(OC(F)F)c2[C@H]2C[C@@H]1c1nc3ccc(Cl)cc3n12 ; C#CC1CN(C(=O)OC(C)(C)C)C1 ; [2H]C([2H])([2H])N1C(=O)c2cccc(OC(F)F)c2[C@H]2C[C@@H]1c1nc3ccc(C#CC4CN(C(=O)OC(C)(C)C)C4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.382 mmol) and tert-butyl 3-ethynylazetidine-1-carboxylate (346 mg, 1.910 mmol) in ACN (5 mL) were added SPhos Pd Gen.3 (60 mg, 0.076 mmol), SPhos (31 mg, 0.076 mmol) and K₂CO₃ (264 mg, 1.910 mmol) at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (10/1) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)azetidine-1-carboxylate (90 mg, 44%) as a light yellow solid. MS ESI calculated for C₂₉H₂₅D₃F₂N₄O₄[M+H]⁺, 538.23. found 538.25.

### Example 69: (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one

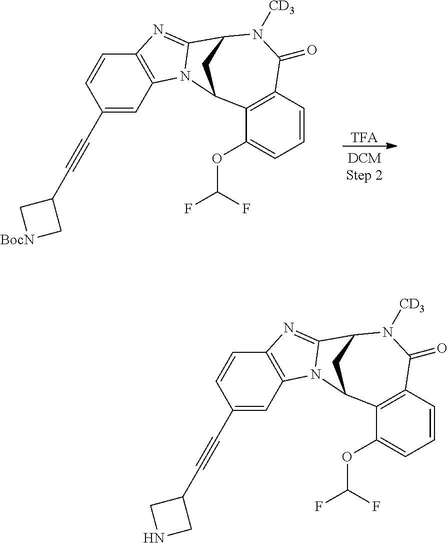 [SMILES: "TFA" ; [2H]C([2H])([2H])N1C(=O)c2cccc(OC(F)F)c2[C@H]2C[C@@H]1c1nc3ccc(C#CC4CN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CNC4)cc3n12]

To a solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)azetidine-1-carboxylate (90 mg, 0.167 mmol) in DCM (3.5 mL) was added TFA (1.5 mL) and stirred for 1 h at room temperature. The resulting mixture was concentrated under vacuum. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (25 mg, 33%) as a white solid. MS ESI calculated for C₂₄H₁₇D₃F₂N₄O₂ [M+H]⁺, 438.17 found 438.05. ¹H NMR (400 MHz, Chloroform-d) δ 8.57-8.41 (m, 1H), 7.61 (d, J=8.5 Hz, 1H), 7.50 (d, J=1.5 Hz, 1H), 7.41 (t, J=8.2 Hz, 1H), 7.38-7.28 (m, 2H), 6.85 (t, J=73.3 Hz, 1H), 6.19 (d, J=7.3 Hz, 1H), 4.93 (d, J=7.1 Hz, 1H), 3.96-3.65 (m, 5H), 3.52-3.38 (m, 1H), 2.85 (d, J=13.6 Hz, 1H). ¹⁹F NMR (377 MHz, Chloroform-d) δ −79.89, −80.33, −81.12, −81.57.

### Example 70: (7R,14R)-11-(3-(tert-butylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

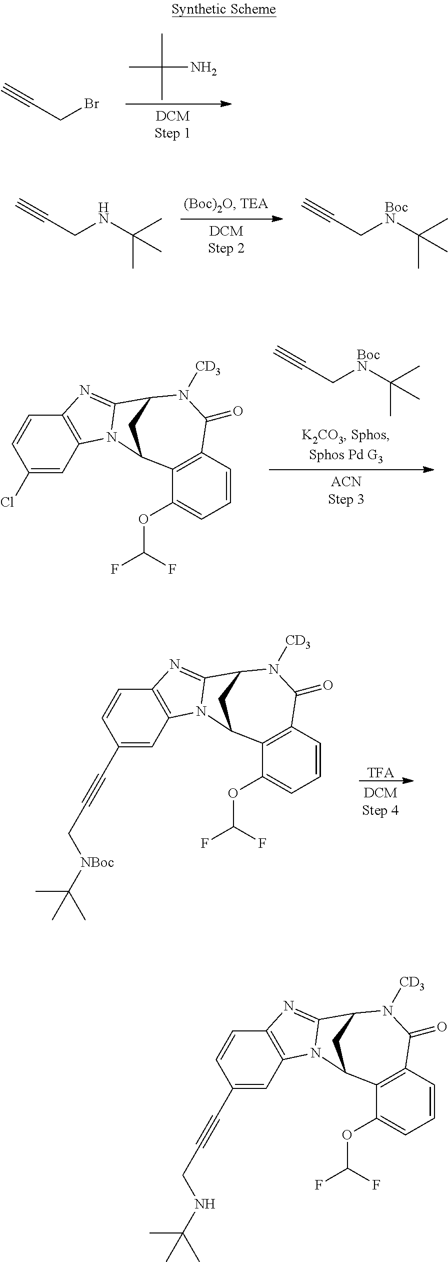

### Preparation 70A: N-(tert-butyl)prop-2-yn-1-amine

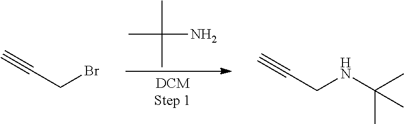

To a stirred solution of 2-methylpropan-2-amine (5 mL) in DCM (15 mL) was added 3-bromoprop-1-yne (1.33 mL, 15.429 mmol) dropwise at room temperature under nitrogen atmosphere. The resulting mixture was stirred for 1 days at room temperature under nitrogen atmosphere. The reaction was quenched with water (20 mL). The aqueous layer was extracted with CH₂Cl₂ (3×40 mL). The combined organic layers were washed with brine (3×30 mL), dried over anhydrous Na₂SO₄. After filtration, the filtrate was concentrated under reduced pressure. The crude product was used in the next step directly without further purification.

### Preparation 70B: tert-butyl tert-butyl(prop-2-yn-1-yl)carbamate

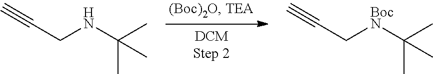

To a stirred solution of N-(tert-butyl)prop-2-yn-1-amine (1.00 g, 8.994 mmol) and TEA (3.8 mL, 26.982 mmol) in DCM (30 mL) was added (Boc)₂O (2.1 mL, 9.893 mmol) dropwise at room temperature. The resulting mixture was stirred for 3 h at room temperature. The mixture was concentrated under reduced pressure. The residue was purified by silica gel column chromatography, eluted with CH₂Cl₂/PE (5/1) to afford tert-butyl tert-butyl(prop-2-yn-1-yl)carbamate (710 mg, 37%) as pink oil. ¹H NMR (400 MHz, Chloroform-d) δ 4.09 (d, J=2.4 Hz, 2H), 2.16 (t, J=2.4 Hz, 1H), 1.49 (s, 9H), 1.46 (s, 9H).

### Preparation 70C: tert-butyl tert-butyl(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)carbamate

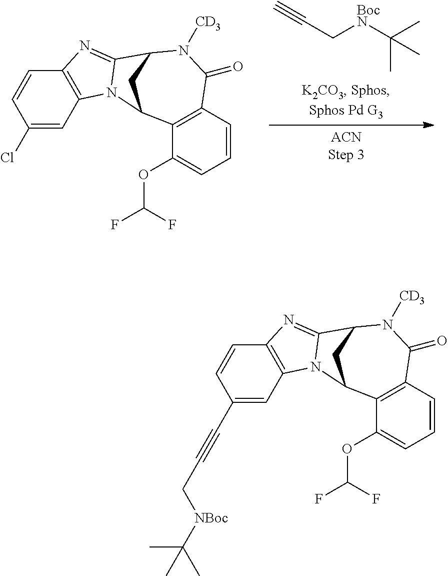 [SMILES: [2H]C([2H])([2H])N1C(=O)c2cccc(OC(F)F)c2[C@H]2C[C@@H]1c1nc3ccc(Cl)cc3n12 ; C#CCN(C(=O)OC(C)(C)C)C(C)(C)C ; [2H]C([2H])([2H])N1C(=O)c2cccc(OC(F)F)c2[C@H]2C[C@@H]1c1nc3ccc(C#CCN(C(=O)OC(C)(C)C)C(C)(C)C)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and tert-butyl tert-butyl(prop-2-yn-1-yl)carbamate (188 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) in portions at room temperature under nitrogen atmosphere. The resulting mixture was stirred for 4 h at 80° C. under nitrogen atmosphere. The mixture was concentrated under reduced pressure. The residue was purified by silica gel column chromatography, eluted with CH₂Cl₂/MeOH (9/1) to afford tert-butyl tert-butyl(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)carbamate (81 mg, 80%) as yellow solid. MS ESI calculated for C₃₁H₃₁D₃F₂N₄O₄ [M+H]⁺, 568.27 found 568.30.

### Example 70: (7R,14R)-11-(3-(tert-butylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

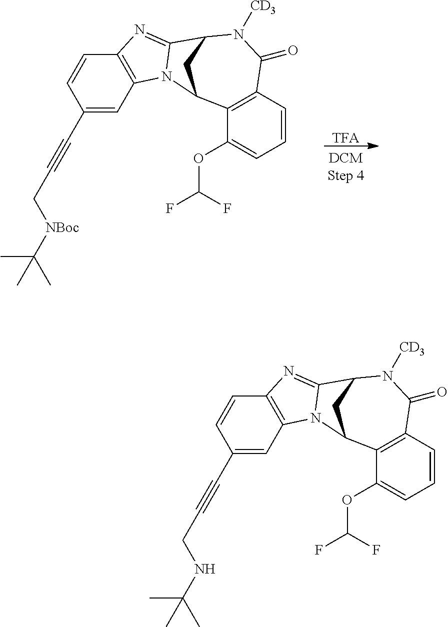 [SMILES: "TFA" ; [2H]C([2H])([2H])N1C(=O)c2cccc(OC(F)F)c2[C@H]2C[C@@H]1c1nc3ccc(C#CCN(C(=O)OC(C)(C)C)C(C)(C)C)cc3n12 ; [2H]C([2H])([2H])N1C(=O)c2cccc(OC(F)F)c2[C@H]2C[C@@H]1c1nc3ccc(C#CCNC(C)(C)C)cc3n12]

A solution of tert-butyl tert-butyl(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)carbamate (81 mg, 0.143 mmol) in DCM (5 mL) and TFA (1 mL) was stirred for 1 h at room temperature. The mixture was concentrated under reduced pressure. The residue was purified by Prep-HPLC with the following conditions (Column: XBridge Prep Phenyl OBD Column 19*250 mm, 5m; Mobile Phase A: Water (10 mmol/L NH₄HCO₃), Mobile Phase B: ACN; Flow rate: 60 mL/min; Gradient: 27% B to 42% B in 10 min; Wave Length:254 nm/220 nm; RT1(min): 9.68) to afford (7R,14R)-11-(3-(tert-butylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (9 mg, 13%) as white solid. MS ESI calculated for C₂₆H₂₃D₃F₂N₄O₂[M+H]⁺, 468.22 found 468.30. ¹H NMR (400 MHz, Chloroform-d) δ 8.52-8.43 (m, 1H), 7.60 (d, J=8.5 Hz, 1H), 7.51 (d, J=3.7 Hz, 1H), 7.44-7.39 (m, 1H), 7.37-7.27 (m, 2H), 6.83 (t, J=72.7 Hz, 1H), 6.20 (d, J=7.3 Hz, 1H), 4.93 (d, J=7.2 Hz, 1H), 3.62 (d, J=3.5 Hz, 2H), 3.48-3.34 (m, 1H), 2.85 (d, J=13.5 Hz, 1H), 1.18 (s, 9H). ¹⁹F NMR (376 MHz, Chloroform-d) δ −79.85, −80.30, −81.13, −81.57.

### Example 71: (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one

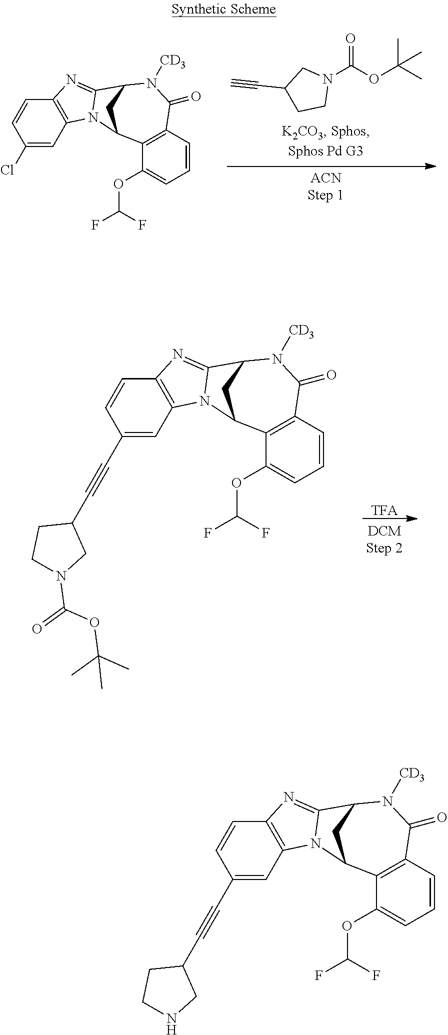

### Preparation 71A: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate

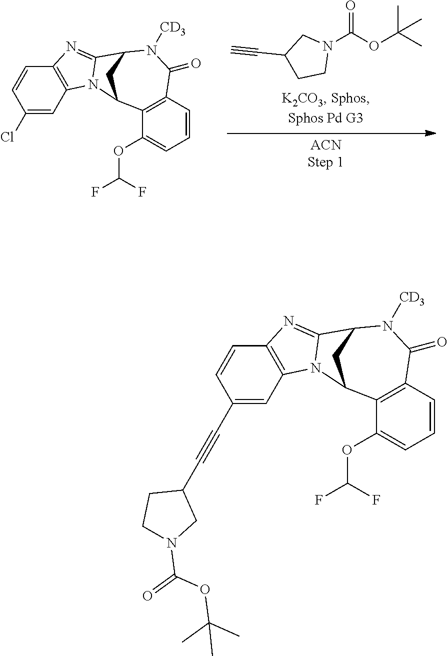

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (120 mg, 0.305 mmol), tert-butyl 3-ethynylpyrrolidine-1-carboxylate (298 mg, 1.525 mmol), K₂CO₃ (211 mg, 1.525 mmol) and SPhos (25 mg, 0.061 mmol) in ACN (2 mL) was added SPhos Pd Gen.3 (48 mg, 0.061 mmol) at room temperature under nitrogen atmosphere. The mixture was stirred for 16 h at 80° C. The resulting mixture was concentrated under vacuum. The residue was purified by silica gel column chromatography, eluted with CH₂Cl₂/MeOH (0~10%) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (120 mg, 71%) as a white solid. MS ESI calculated for C₃₀H₂₇D₃F₂N₄O₄ [M+H]⁺, 552.24. found 552.20. ¹H NMR (400 MHz, Chloroform-d) δ 8.53-8.46 (m, 1H), 7.67 (d, J=8.5 Hz, 1H), 7.53 (s, 1H), 7.45 (t, J=8.2 Hz, 1H), 7.39-7.30 (m, 2H), 6.86 (t, J=72.6 Hz, 1H), 6.29-6.24 (m, 1H), 5.17-5.12 (m, 1H), 3.79-3.30 (m, 5H), 3.29-3.14 (m, 1H), 2.90 (d, J=11.7 Hz, 1H), 2.28-2.18 (m, 1H), 2.10-2.02 (m, 1H), 1.47 (s, 9H).

### Example 71: (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one

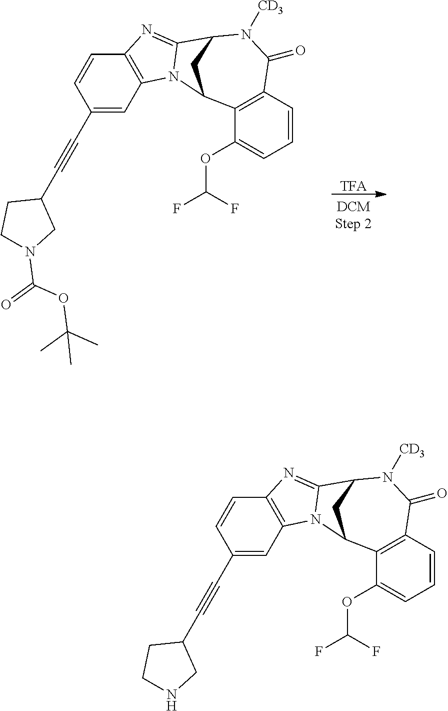 [SMILES: "TFA" ; [2H]C([2H])([2H])N1C(=O)c2cccc(OC(F)F)c2[C@H]2C[C@@H]1c1nc3ccc(C#CC4CCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CCNC4)cc3n12]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (120 mg, 0.218 mmol) in DCM (2 mL) was added TFA (0.4 mL) dropwise at room temperature. The resulting mixture was stirred for 1 h at room temperature. The mixture was concentrated under vacuum. The residue was purified by reversed-phase flash chromatography with the following conditions: column, C18 silica gel; mobile phase, CH₃CN in Water (10 mmol/L NH₄HCO₃), 30% to 50% gradient in 25 min; detector, 254 nm to afford (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (80 mg, 81%) as a white solid. MS ESI calculated for C₂₅H₁₉D₃F₂N₄O₂[M+H]⁺, 452.19. found 452.10. ¹H NMR (400 MHz, Methanol-d₄) δ 8.40-8.33 (m, 1H), 7.57-7.43 (m, 4H), 7.42-7.03 (m, 2H), 6.34 (d, J=7.1 Hz, 1H), 5.16 (d, J=7.1 Hz, 1H), 3.59-3.47 (m, 1H), 3.31-3.24 (m, 1H), 3.21-3.09 (m, 2H), 3.08-2.99 (m, 1H), 2.98-2.91 (m, 1H), 2.90-2.82 (m, 1H), 2.29-2.16 (m, 1H), 2.01-1.87 (m, 1H). ¹⁹F NMR (377 MHz, Methanol-d₄) δ −83.03, −83.48, −83.56, −84.01.

### Example 72 and 73: (7R,14R)-1-(difluoromethoxy)-11-(3-((1-hydroxypropan-2-yl)oxy)prop-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-(3-(2-hydroxypropoxy)prop-1-yn-1-yl)-6-(methyl-d3)-6,7-dihydro-7,14-methanobenzo[f]benzo[4,5]imidazo[1,2-a][1,4]diazocin-5(14H)-one

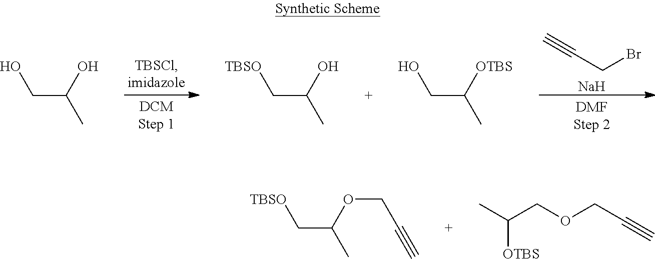

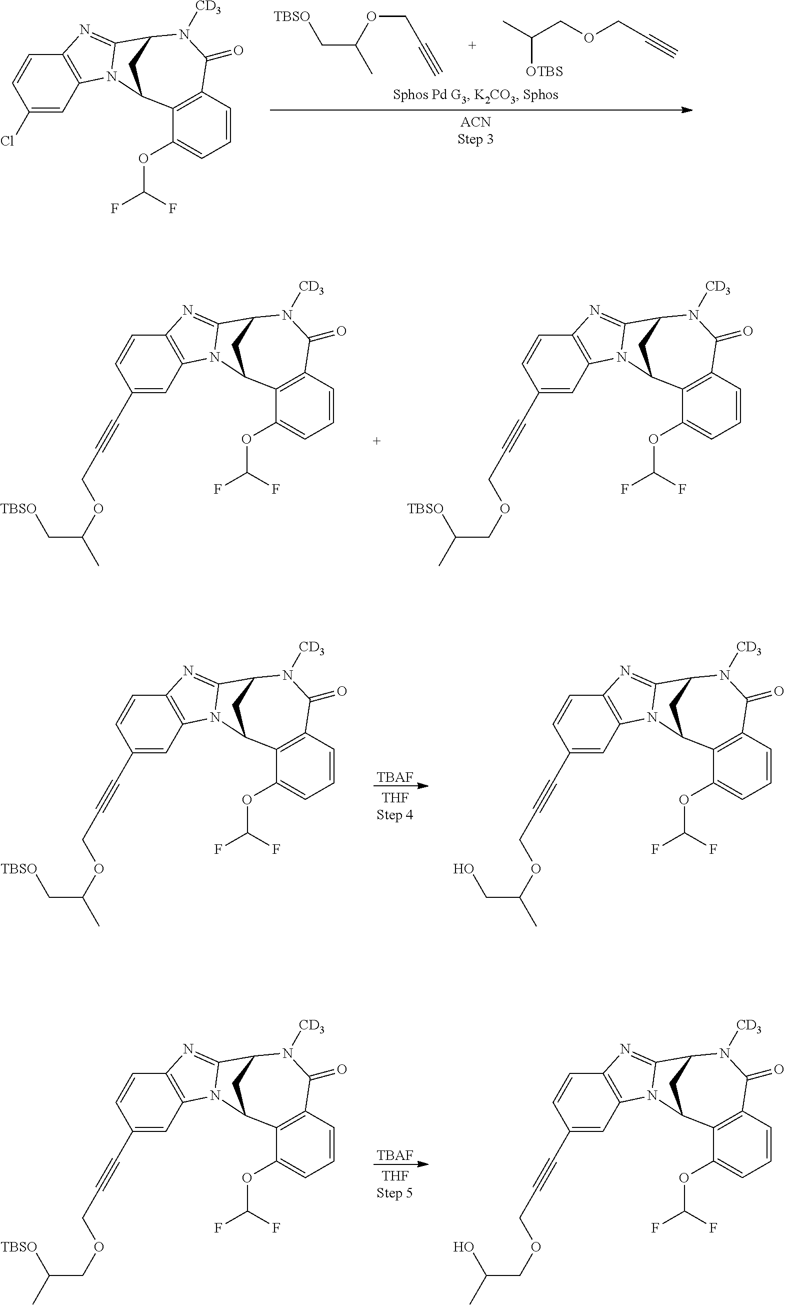

### Preparation 72A: 1-[(tert-butyldimethylsilyl)oxy]propan-2-ol and 2-[(tert-butyldimethylsilyl)oxy]propan-2-ol

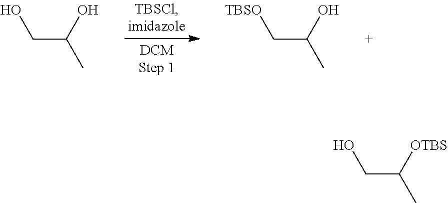

To a stirred solution of 1,2-propanediol (5.00 g, 65.707 mmol) and imidazole (4.47 g, 65.707 mmol) in DCM (80 mL) was added tert-butyl(chloro)dimethylsilane (9.90 g, 65.707 mmol) in DCM (80 mL) dropwise at 0° C. The resulting mixture was stirred for 2 h at room temperature. The mixture was filtered, the filter cake was washed with CH₂Cl₂ (3×30 mL). The filtrate was concentrated under reduced pressure to afford the mixture of 1-[(tert-butyldimethylsilyl)oxy]propan-2-ol and 2-[(tert-butyldimethylsilyl)oxy]propan-2-ol (12.10 g, 96%) as a colorless oil. MS ESI calculated for C₉H₂₂O₂Si [M+H]⁺, 191.14. found N/A. ¹H NMR (400 MHz, Chloroform-d) δ 3.87-3.77 (m, 1H), 3.61-3.58 (m, 1H), 3.37-3.32 (m, 1H), 1.18 (d, J=6.4 Hz, 1H), 1.12 (d, J=6.3 Hz, 3H), 0.91 (s, 9H), 0.08 (s, 6H).

### Preparation 72B: tert-butyldimethyl[2-(prop-2-yn-1-yloxy)propoxy]silane and tert-butyldimethyl[1-(prop-2-yn-1-yloxy)propoxy]silane

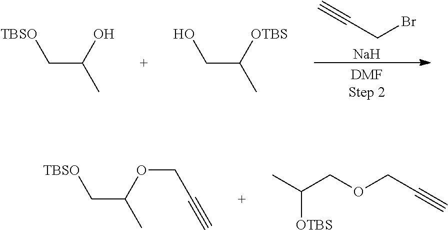

To a stirred solution of propargyl bromide (2.50 g, 21.014 mmol) and the mixture of 1-[(tert-butyldimethylsilyl)oxy]propan-2-ol and 2-[(tert-butyldimethylsilyl)oxy]propan-2-ol (2.00 g, 10.506 mmol) in DMF (35 mL) was added NaH (0.84 g, 21.014 mmol, 60%) at 0° C. The resulting mixture was stirred for 2 h at room temperature. The reaction was quenched with water at 0° C. The resulting mixture was extracted with EtOAc (3×200 mL). The combined organic layers were washed with brine (200 mL), dried over anhydrous Na₂SO₄. After filtration, the filtrate was concentrated under reduced pressure. The residue was purified by silica gel column chromatography, eluted with PE/EA (4:1) to afford the mixture of tert-butyldimethyl[2-(prop-2-yn-1-yloxy)propoxy]silane and tert-butyldimethyl[1-(prop-2-yn-1-yloxy)propoxy]silane (1.27 g, 53%) as a yellow oil. MS ESI calculated for C₁₂H₂₄O₂Si [M+H]⁺, 229.15. found N/A. ¹H NMR (400 MHz, Chloroform-d) δ 4.19 (d, J=2.2 Hz, 1H), 4.11 (s, 1H), 3.67-3.57 (m, 1H), 3.47-3.42 (m, 1H), 3.39-3.27 (m, 1H), 2.36-2.31 (m, 1H), 1.09 (d, J=6.1 Hz, 3H), 0.83 (d, J=4.2 Hz, 9H), 0.00 (d, J=2.2 Hz, 6H).

### Preparation 72C and 73C: (7R,14R)-11-(3-((1-((tert-butyldimethylsilyl)oxy)propan-2-yl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-(3-(2-((tert-butyldimethylsilyl)oxy)propoxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

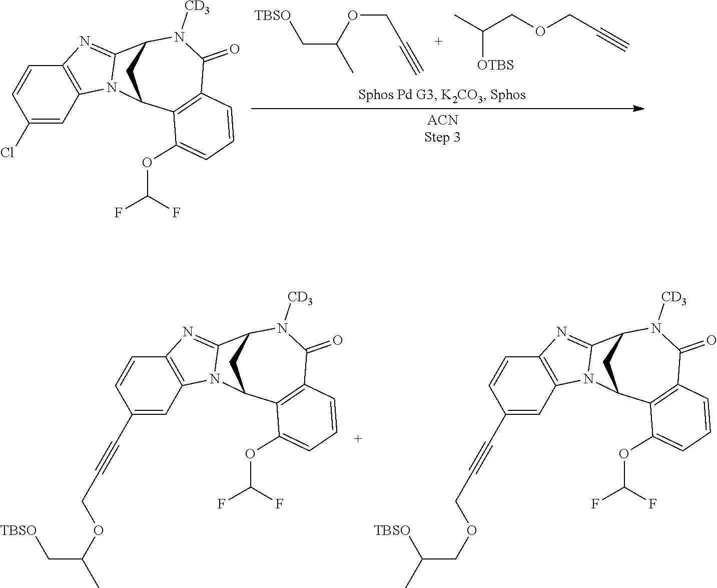 [SMILES: [2H]C([2H])([2H])N1C(=O)c2cccc(OC(F)F)c2[C@H]2C[C@@H]1c1nc3ccc(Cl)cc3n12 ; C#CCOC(C)CO[Si](C)(C)C(C)(C)C ; C#CCOCC(C)O[Si](C)(C)C(C)(C)C ; [2H]C([2H])([2H])N1C(=O)c2cccc(OC(F)F)c2[C@H]2C[C@@H]1c1nc3ccc(C#CCOC(C)CO[Si](C)(C)C(C)(C)C)cc3n12 ; [2H]C([2H])([2H])N1C(=O)c2cccc(OC(F)F)c2[C@H]2C[C@@H]1c1nc3ccc(C#CCOCC(C)O[Si](C)(C)C(C)(C)C)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (130 mg, 0.331 mmol), the mixture of tert-butyldimethyl[1-(prop-2-yn-1-yloxy)propoxy]silane and tert-butyldimethyl[2-(prop-2-yn-1-yloxy)propoxy]silane (302 mg, 1.324 mmol) in ACN (2 mL) were added SPhos Pd Gen.3 (52 mg, 0.066 mmol), SPhos (27 mg, 0.066 mmol) and K₂CO₃ (229 mg, 1.655 mmol) at room temperature under nitrogen atmosphere. The resulting mixture was stirred for 2 h at 80° C. under nitrogen atmosphere. The mixture was allowed to cool down to room temperature, and concentrated under reduced pressure. The residue was purified by silica gel column chromatography, eluted with CH₂Cl₂/MeOH (10/1) to afford (7R,14R)-11-(3-((1-((tert-butyldimethylsilyl)oxy)propan-2-yl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (48 mg, 25%) as a light yellow solid. and (7R,14R)-11-(3-(2-((tert-butyldimethylsilyl)oxy)propoxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (45 mg, 23%) as a light yellow solid. MS ESI calculated for C₃₁H₃₄D₃F₂N₃O₄Si [M+H]⁺, 585.27. found 585.35.

### Example 72: (7R,14R)-1-(difluoromethoxy)-11-(3-((1-hydroxypropan-2-yl)oxy)prop-1-yn-1-yl)-6-(methyl-d3)-6,7-dihydro-7,14-methanobenzo[f]benzo[4,5]imidazo[1,2-a][1,4]diazocin-5(14H)-one

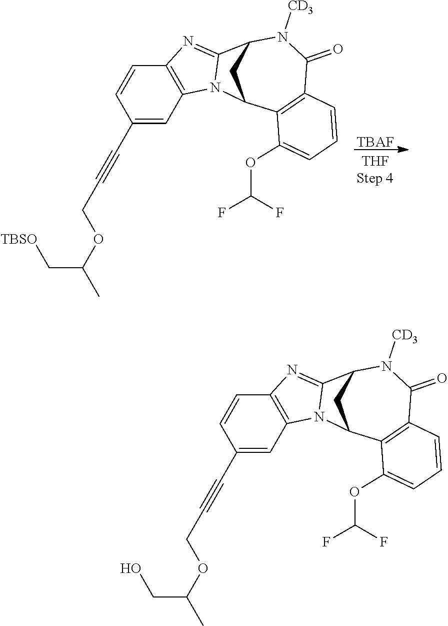

A solution of (7R,14R)-11-(3-((1-((tert-butyldimethylsilyl)oxy)propan-2-yl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (45 mg, 0.077 mmol) and TBAF (92 uL, 0.092 mmol) in THF (1 mL) was stirred for 1 h at room temperature. The resulting mixture was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-(3-((1-hydroxypropan-2-yl)oxy)prop-1-yn-1-yl)-6-(methyl-d3)-6,7-dihydro-7,14-methanobenzo[f]benzo[4,5]imidazo[1,2-a][1,4]diazocin-5(14H)-one (6 mg, 16%) as a white solid. MS ESI calculated for C₂₅H₂₀D₃F₂N₃O₄[M+H]⁺, 471.18. found 471.15. ¹H NMR (400 MHz, Chloroform-d) δ 8.48 (d, J=7.4 Hz, 1H), 7.65 (d, J=8.4 Hz, 1H), 7.57 (s, 1H), 7.43 (t, J=8.2 Hz, 1H), 7.37-7.31 (m, 2H), 7.07-6.68 (m, 1H), 6.24 (d, J=7.3 Hz, 1H), 5.01 (d, J=7.1 Hz, 1H), 4.52 (d, J=15.9 Hz, 1H), 4.42 (d, J=15.9 Hz, 1H), 3.92-3.81 (m, 1H), 3.70-3.63 (m, 1H), 3.58-3.40 (m, 2H), 2.87 (d, J=13.6 Hz, 1H), 1.21 (d, J=6.3 Hz, 3H). ¹⁹F NMR (377 MHz, Chloroform-d) δ −79.93, −80.08, −80.38, −80.52, −81.24, −81.35, −81.69, −81.79.

### Example 73: (7R,14R)-1-(difluoromethoxy)-11-(3-(2-hydroxypropoxy)prop-1-yn-1-yl)-6-(methyl-d3)-6,7-dihydro-7,14-methanobenzo[f]benzo[4,5]imidazo[1,2-a][1,4]diazocin-5(14H)-one

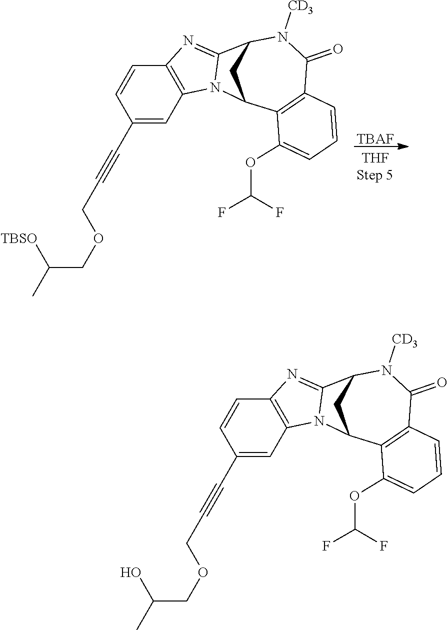

A solution of (7R,14R)-11-(3-(2-((tert-butyldimethylsilyl)oxy)propoxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (45 mg, 0.077 mmol) and TBAF (92 uL, 0.092 mmol) in THF (1 mL) was stirred for 1 h at room temperature. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 30% to 70% gradient in 20 min; detector, 254 nm. This resulted in (7R,14R)-1-(difluoromethoxy)-11-(3-(2-hydroxypropoxy)prop-1-yn-1-yl)-6-(methyl-d3)-6,7-dihydro-7,14-methanobenzo[f]benzo[4,5]imidazo[1,2-a][1,4]diazocin-5(14H)-one (10 mg, 28%) as a white solid. MS ESI calculated for C₂₅H₂₀D₃F₂N₃O₄[M+H]⁺, 471.18. found 471.15. ¹H NMR (400 MHz, Chloroform-d) δ 8.51-8.46 (m, 1H), 7.65 (d, J=8.5 Hz, 1H), 7.57 (s, 1H), 7.43 (t, J=8.2 Hz, 1H), 7.37-7.31 (m, 2H), 7.05-6.66 (m, 1H), 6.23 (d, J=7.2 Hz, 1H), 4.99 (d, J=6.9 Hz, 1H), 4.44 (d, J=3.7 Hz, 2H), 4.12-3.95 (m, 1H), 3.65-3.62 (m, 1H), 3.50-3.37 (m, 2H), 2.87 (d, J=13.5 Hz, 1H), 1.20 (d, J=6.4 Hz, 3H). ¹⁹F NMR (377 MHz, Chloroform-d) δ −80.01, −80.03, −80.46, −80.48, −81.08, −81.09, −81.53, −81.53.

### Example 74: (7R,14R)-1-(difluoromethoxy)-11-(3-isopropoxyprop-1-yn-1-yl)-6-(methyl-d3)-6,7-dihydro-7,14-methanobenzo[f]benzo[4,5]imidazo[1,2-a][1,4]diazocin-5(14H)-one

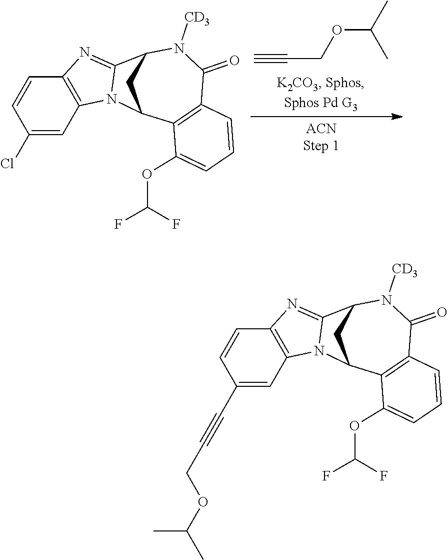

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and 3-isopropoxyprop-1-yne (100 mg, 1.020 mmol) in ACN (1 mL) were added K₂CO₃ (141 mg, 1.020 mmol), SPhos Pd Gen.3 (32 mg, 0.041 mmol) and SPhos (17 mg, 0.041 mmol) in portions at room temperature under nitrogen atmosphere. The resulting mixture was stirred for overnight at 80° C. under nitrogen atmosphere. The mixture was concentrated under vacuum. The residue was purified by silica gel column chromatography, eluted with CH₂Cl₂/MeOH (8/1) followed by reversed-phase flash chromatography with the following conditions: column, C18 silica gel; mobile phase, CH₃CN in Water (10 mmol/L NH₄HCO₃), 40% to 60% gradient in 10 min; detector, 254 nm to afford (7R,14R)-1-(difluoromethoxy)-11-(3-isopropoxyprop-1-yn-1-yl)-6-(methyl-d3)-6,7-dihydro-7,14-methanobenzo[f]benzo[4,5]imidazo[1,2-a][1,4]diazocin-5(14H)-one (6 mg, 7%) as yellow solid. MS ESI calculated for C₂₅H₂₀D₃F₂N₃O₃[M+H]⁺, 455.19 found 455.35. ¹H NMR (400 MHz, Chloroform-d) δ 8.53-8.42 (m, 1H), 7.64 (d, J=8.5 Hz, 1H), 7.57 (d, J=1.4 Hz, 1H), 7.43 (t, J=8.2 Hz, 1H), 7.38-7.33 (m, 2H), 7.10-6.65 (m, 1H), 6.23 (d, J=5.9 Hz, 1H), 5.08-4.94 (m, 1H), 4.39 (s, 2H), 3.96-3.83 (m, 1H), 3.53-3.40 (m, 1H), 2.87 (d, J=12.9 Hz, 1H), 1.24 (d, J=6.1 Hz, 6H). ¹⁹F NMR (377 MHz, Chloroform-d) δ −80.01, −80.45, −81.13, −81.57.

### Example 75: (7R,14R)-1-(difluoromethoxy)-6-(methyl-d3)-11-(3-methylbut-1-yn-1-yl)-6,7-dihydro-7,14-methanobenzo[f]benzo[4,5]imidazo[1,2-a][1,4]diazocin-5(14H)-one

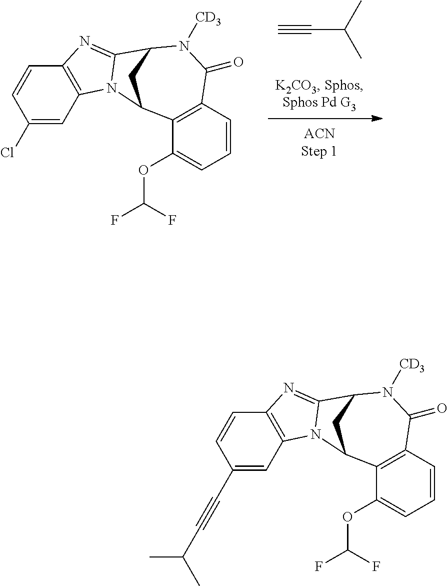

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and 3-methylbut-1-yne (69 mg, 1.020 mmol) in ACN (1 mL) were added K₂CO₃ (141 mg, 1.020 mmol), SPhos Pd Gen.3 (32 mg, 0.041 mmol) and SPhos (17 mg, 0.041 mmol) in portions at room temperature under nitrogen atmosphere. The resulting mixture was stirred for 3 h at 80° C. under nitrogen atmosphere. The mixture was concentrated under vacuum. The residue was purified by silica gel column chromatography, eluted with CH₂Cl₂/MeOH (9/1) followed by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min; Gradient: 43% B to 63% B in 10 min; Wave Length: 254 nm/220 nm; RT1(min): 8.52) to afford (7R,14R)-1-(difluoromethoxy)-6-(methyl-d3)-11-(3-methylbut-1-yn-1-yl)-6,7-dihydro-7,14-methanobenzo[f]benzo[4,5]imidazo[1,2-a][1,4]diazocin-5(14H)-one (51 mg, 59%) as a white solid. MS ESI calculated for C₂₄H₁₈D₃F₂N₃O₂[M+H]⁺, 425.18 found 425.30. ¹H NMR (400 MHz, Chloroform-d) δ 8.53-8.45 (m, 1H), 7.63 (d, J=8.5 Hz, 1H), 7.57-7.49 (m, 1H), 7.44 (t, J=8.2 Hz, 1H), 7.39-7.30 (m, 2H), 7.06-6.65 (m, 1H), 6.24 (d, J=7.2 Hz, 1H), 5.06 (d, J=7.1 Hz, 1H), 3.53-3.42 (m, 1H), 2.88 (d, J=13.6 Hz, 1H), 2.84-2.73 (m, 1H), 1.28 (d, J=6.9 Hz, 6H). ¹⁹F NMR (377 MHz, Chloroform-d) δ −80.00, −80.45, −81.31, −81.76.

### Example 76: (7R,14R)-1-(difluoromethoxy)-6-(methyl-d3)-11-((1-methylpyrrolidin-3-yl)ethynyl)-6,7-dihydro-7,14-methanobenzo[f]benzo[4,5]imidazo[1,2-a][1,4]diazocin-5(14H)-one

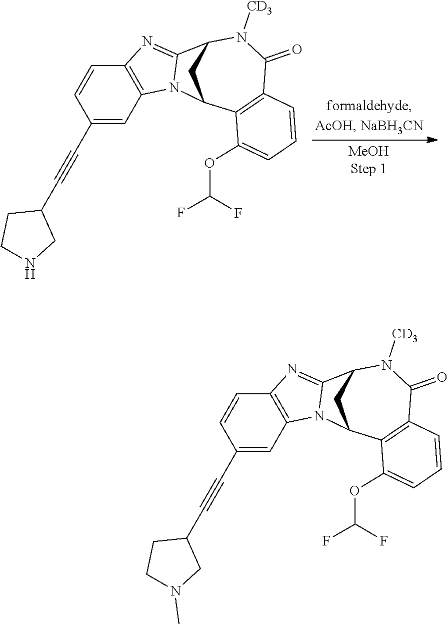 [SMILES: [2H]C([2H])([2H])N1C(=O)c2cccc(OC(F)F)c2[C@H]2C[C@@H]1c1nc3ccc(C#CC4CCNC4)cc3n12 ; [2H]C([2H])([2H])N1C(=O)c2cccc(OC(F)F)c2[C@H]2C[C@@H]1c1nc3ccc(C#CC4CCN(C)C4)cc3n12]

To a stirred mixture of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (35 mg, 0.078 mmol) in MeOH (1 mL) were added formaldehyde (9 mg, 0.117 mmol, 37%) and AcOH (14 mg, 0.234 mmol) at room temperature. The resulting mixture was stirred for 30 min at room temperature. To the above mixture was added NaBH₃CN (7 mg, 0.117 mmol) at room temperature. The resulting mixture was stirred for additional 2 h at room temperature. The residue was purified by reversed-phase flash chromatography with the following conditions: column, C18 silica gel; mobile phase, CH₃CN in Water (10 mmol/L NH₄HCO₃), 30% to 50% gradient in 20 min; detector, 254 nm to afford (7R,14R)-1-(difluoromethoxy)-6-(methyl-d3)-11-((1-methylpyrrolidin-3-yl)ethynyl)-6,7-dihydro-7,14-methanobenzo[f]benzo[4,5]imidazo[1,2-a][1,4]diazocin-5(14H)-one (10 mg, 26%) as a white solid. MS ESI calculated for C₂₆H₂₁D₃F₂N₄O₂ [M+H]⁺, 466.21. found 466.40. ¹H NMR (400 MHz, Methanol-d₄) δ 8.39-8.33 (m, 1H), 7.56-7.43 (m, 4H), 7.42-7.03 (m, 2H), 6.34 (d, J=7.1 Hz, 1H), 5.17 (d, J=7.2 Hz, 1H), 3.61-3.48 (m, 1H), 3.29-3.19 (m, 1H), 3.02-2.94 (m, 1H), 2.90-2.83 (m, 1H), 2.77-2.56 (m, 3H), 2.39 (s, 3H), 2.38-2.25 (m, 1H), 2.05-1.94 (m, 1H). ¹⁹F NMR (376 MHz, Methanol-d₄) δ −83.10, −83.55, −83.56, −84.01.

### Example 77: (7R,14R)-1-(difluoromethoxy)-11-(4-methoxybut-1-yn-1-yl)-6-(methyl-d3)-6,7-dihydro-7,14-methanobenzo[f]benzo[4,5]imidazo[1,2-a][1,4]diazocin-5(14H)-one

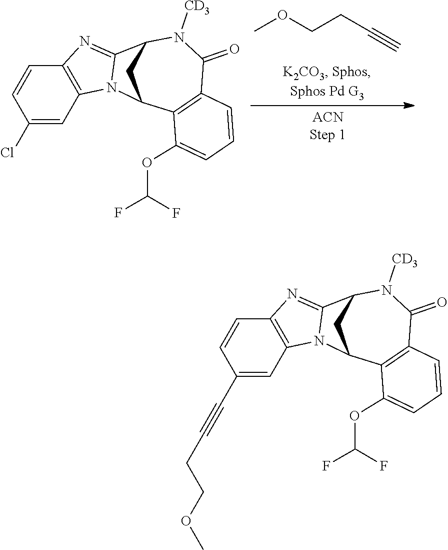

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 4-methoxybut-1-yne (75 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) in portions at room temperature under nitrogen atmosphere. The resulting mixture was stirred for 3 h at 80° C. under nitrogen atmosphere. The mixture was concentrated under vacuum. The residue was purified by silica gel column chromatography, eluted with CH₂Cl₂/MeOH (8/1) followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 27% B to 42% B in 10 min; Wave Length: 254 nm/220 nm; RT1(min): 12.34) to afford (7R,14R)-1-(difluoromethoxy)-11-(4-methoxybut-1-yn-1-yl)-6-(methyl-d3)-6,7-dihydro-7,14-methanobenzo[f]benzo[4,5]imidazo[1,2-a][1,4]diazocin-5(14H)-one (45 mg, 56%) as a white solid. MS ESI calculated for C₂₄H₁₈D₃F₂N₃O₃. [M+H]⁺, 441.17 found 441.30. ¹H NMR (400 MHz, Chloroform-d) δ 8.49-8.39 (m, 1H), 7.59 (d, J=8.4 Hz, 1H), 7.49 (d, J=1.5 Hz, 1H), 7.39 (t, J=8.2 Hz, 1H), 7.34-7.24 (m, 2H), 6.85 (t, J=73.3 Hz, 1H), 6.15 (d, J=7.2 Hz, 1H), 4.90 (d, J=7.1 Hz, 1H), 3.61 (t, J=6.9 Hz, 2H), 3.49-3.31 (m, 4H), 2.81 (d, J=13.6 Hz, 1H), 2.71 (t, J=6.9 Hz, 2H). ¹⁹F NMR (377 MHz, Chloroform-d) δ −79.92, −80.36, −81.17, −81.62.

### Example 78: (7R,14R)-1-(difluoromethoxy)-11-(3-ethoxybut-1-yn-1-yl)-6-(methyl-d3)-6,7-dihydro-7,14-methanobenzo[f]benzo[4,5]imidazo[1,2-a][1,4]diazocin-5(14H)-one

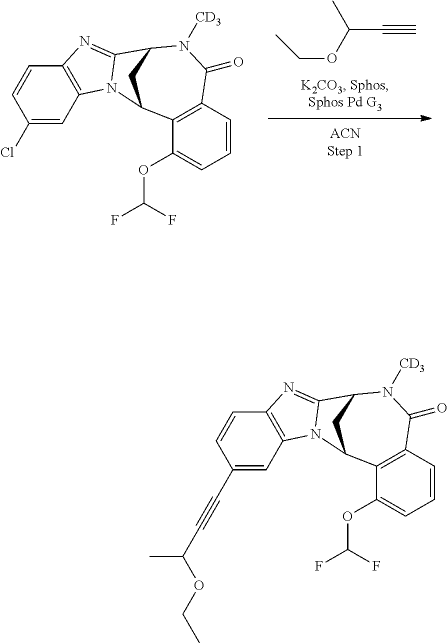

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 3-ethoxybut-1-yne (75 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) in portions at room temperature under nitrogen atmosphere. The resulting mixture was stirred for overnight at 80° C. under nitrogen atmosphere. The mixture was concentrated under vacuum. The residue was purified by silica gel column chromatography, eluted with CH₂Cl₂/MeOH (9/1) followed by reversed-phase flash chromatography with the following conditions: column, C18 silica gel; mobile phase, CH₃CN in water (10 mmol/L NH₄HCO₃), 50% to 60% gradient in 10 min; detector, 254 nm to afford (7R,14R)-1-(difluoromethoxy)-11-(3-ethoxybut-1-yn-1-yl)-6-(methyl-d3)-6,7-dihydro-7,14-methanobenzo[f]benzo[4,5]imidazo[1,2-a][1,4]diazocin-5(14H)-one (5 mg, 6%) as a yellow solid. MS ESI calculated for C₂₅H₂₀D₃F₂N₃O₃[M+H]⁺, 455.19 found 455.30. ¹H NMR (400 MHz, Chloroform-d) δ 8.52-8.44 (m, 1H), 7.64 (d, J=8.4 Hz, 1H), 7.56 (s, 1H), 7.43 (t, J=8.2 Hz, 1H), 7.38-7.29 (m, 2H), 6.85 (t, J=72.7 Hz, 1H), 6.23 (d, J=7.0 Hz, 1H), 5.00 (d, J=6.9 Hz, 1H), 4.41 (q, J=6.6 Hz, 1H), 3.92-3.82 (m, 1H), 3.59-3.40 (m, 2H), 2.87 (d, J=13.5 Hz, 1H), 1.54 (d, J=6.6 Hz, 3H), 1.27 (t, J=7.0 Hz, 3H). ¹⁹F NMR (377 MHz, Chloroform-d) δ −80.09, −80.54, −81.22, −81.66.

### Example 79: (7R,14R)-1-(difluoromethoxy)-6-(methyl-d3)-11-(3-phenoxyprop-1-yn-1-yl)-6,7-dihydro-7,14-methanobenzo[f]benzo[4,5]imidazo[1,2-a][1,4]diazocin-5(14H)-one

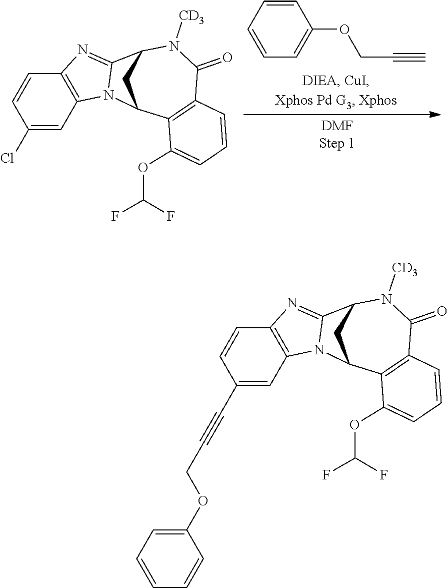

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and (prop-2-yn-1-yloxy)benzene (81 mg, 0.612 mmol) in DMF (1 mL) were added DIPEA (132 mg, 1.020 mmol), XPhos (19 mg, 0.041 mmol), CuI (2 mg, 0.010 mmol) and XPhos Pd G3 (17 mg, 0.020 mmol) in portions at room temperature under nitrogen atmosphere. The resulting mixture was stirred for overnight at 110° C. under nitrogen atmosphere. The mixture was concentrated under vacuum. The residue was purified by reversed-phase flash chromatography with the following conditions: column, C18 silica gel; mobile phase, CH₃CN in Water (10 mmol/L NH₄HCO₃), 20% to 30% gradient in 10 min; detector, 254 nm to afford (7R,14R)-1-(difluoromethoxy)-6-(methyl-d3)-11-(3-phenoxyprop-1-yn-1-yl)-6,7-dihydro-7,14-methanobenzo[f]benzo[4,5]imidazo[1,2-a][1,4]diazocin-5(14H)-one (7 mg, 7%) as a white solid. MS ESI calculated for C₂₈H₁₈D₃F₂N₃O₃ [M+H]⁺, 489.17 found 489.15. ¹H NMR (400 MHz, Chloroform-d) δ 8.48 (d, J=8.2 Hz, 1H), 7.64 (d, J=8.5 Hz, 1H), 7.55 (s, 1H), 7.48-7.39 (m, 1H), 7.33 (t, J=7.8 Hz, 4H), 7.06-6.62 (m, 4H), 6.22 (d, J=6.7 Hz, 1H), 4.99 (d, J=6.6 Hz, 1H), 4.94 (d, J=2.2 Hz, 2H), 3.52-3.40 (m, 1H), 2.87 (d, J=13.3 Hz, 1H). ¹⁹F NMR (377 MHz, Chloroform-d) δ −79.88, −80.33, −81.11, −81.55.

### Example 80: (7R,14R)-1-(difluoromethoxy)-6-(methyl-d3)-11-(oxetan-3-ylethynyl)-6,7-dihydro-7,14-methanobenzo[f]benzo[4,5]imidazo[1,2-a][1,4]diazocin-5(14H)-one

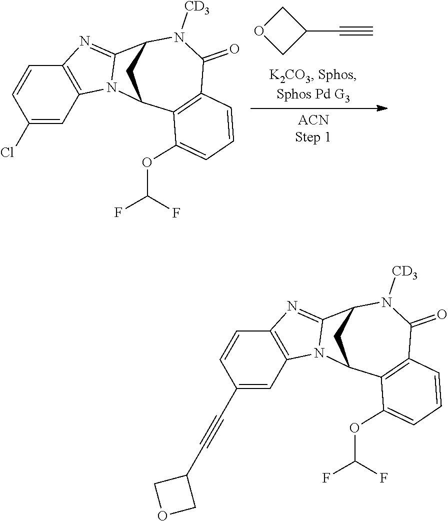

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 3-ethynyloxetane (73 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) in portions at room temperature under nitrogen atmosphere. The resulting mixture was stirred for overnight at 80° C. under nitrogen atmosphere. The residue was purified by silica gel column chromatography, eluted with CH₂Cl₂/MeOH (9/1) followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: MEOH; Flow rate: 60 mL/min; Gradient: 44% B to 59% B in 10 min; Wave Length: 254 nm/220 nm; RT1(min): 14.66) to afford (7R,14R)-1-(difluoromethoxy)-6-(methyl-d3)-11-(oxetan-3-ylethynyl)-6,7-dihydro-7,14-methanobenzo[f]benzo[4,5]imidazo[1,2-a][1,4]diazocin-5(14H)-one (13 mg, 17%) as white solid. MS ESI calculated for C₂₄H₁₆D₃F₂N₃O₃ [M+H]⁺, 439.16 found 439.10. ¹H NMR (400 MHz, Chloroform-d) δ 8.54-8.44 (m, 1H), 7.65 (d, J=8.5 Hz, 1H), 7.53 (s, 1H), 7.48-7.40 (m, 1H), 7.38-7.30 (m, 2H), 6.84 (t, J=72.7 Hz, 1H), 6.23 (d, J=7.2 Hz, 1H), 4.99 (d, J=7.0 Hz, 1H), 4.93-4.87 (m, 2H), 4.86-4.78 (m, 2H), 4.18-4.02 (m, 1H), 3.52-3.39 (m, 1H), 2.87 (d, J=13.5 Hz, 1H). ¹⁹F NMR (377 MHz, Chloroform-d) δ −79.97, −80.42, −81.04, −81.48.

### Example 81: (7R,14R)-1-(difluoromethoxy)-11-(3-(3-hydroxyoxetan-3-yl)prop-1-yn-1-yl)-6-(methyl-d3)-6,7-dihydro-7,14-methanobenzo[f]benzo[4,5]imidazo[1,2-a][1,4]diazocin-5(14H)-one

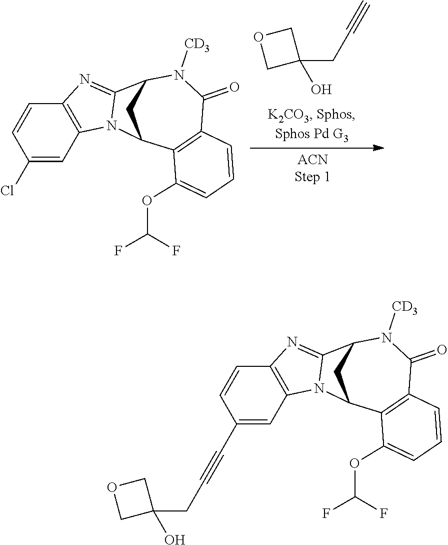

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 3-(prop-2-yn-1-yl)oxetan-3-ol (100 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) in portions at room temperature under nitrogen atmosphere. The resulting mixture was stirred for 2 h at 80° C. under nitrogen atmosphere. The mixture was concentrated under reduce pressure. The residue was purified by silica gel column chromatography, eluted with CH₂Cl₂/MeOH (9/1) followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 20% B to 35% B in 10 min; Wave Length: 254 nm/220 nm; RT1(min): 10.68) to afford (7R,14R)-1-(difluoromethoxy)-11-(3-(3-hydroxyoxetan-3-yl)prop-1-yn-1-yl)-6-(methyl-d3)-6,7-dihydro-7,14-methanobenzo[f]benzo[4,5]imidazo[1,2-a][1,4]diazocin-5(14H)-one (32 mg, 38%) as white solid. MS ESI calculated for C₂₅H₁₈D₃F₂N₃O₄ [M+H]⁺, 469.17 found 469.10. ¹H NMR (400 MHz, Chloroform-d) δ 8.54-8.44 (m, 1H), 7.58 (d, J=8.4 Hz, 1H), 7.53 (d, J=1.4 Hz, 1H), 7.44 (t, J=8.2 Hz, 1H), 7.34 (d, J=8.2 Hz, 1H), 7.29-7.24 (m, 1H), 6.89 (t, J=72.7 Hz, 1H), 6.24 (d, J=7.0 Hz, 1H), 5.08 (d, J=6.8 Hz, 1H), 4.82-4.68 (m, 2H), 4.61-4.51 (m, 2H), 3.56-3.41 (m, 1H), 3.03 (s, 2H), 2.88 (d, J=13.5 Hz, 1H). ¹⁹F NMR (377 MHz, Chloroform-d) δ −80.28, −80.72, −81.26, −81.71.

### Example 82: (7R,14R)-1-(difluoromethoxy)-11-(4-hydroxy-4-methylpent-1-yn-1-yl)-6-(methyl-d3)-6,7-dihydro-7,14-methanobenzo[f]benzo[4,5]imidazo[1,2-a][1,4]diazocin-5(14H)-one

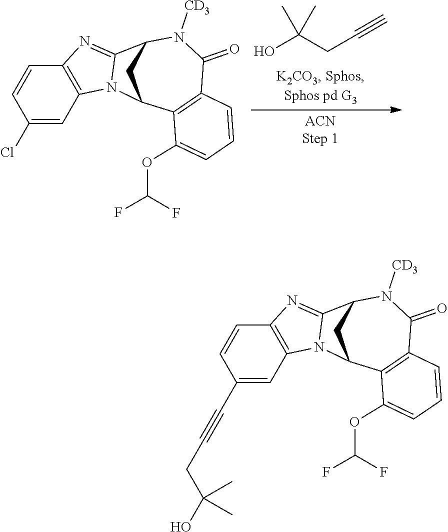

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 2-methylpent-4-yn-2-ol (87 mg, 0.890 mmol) in ACN (1 mL) were added SPhos Pd Gen.3 (28 mg, 0.036 mmol), SPhos (15 mg, 0.036 mmol) and K₂CO₃ (123 mg, 0.890 mmol) at room temperature under nitrogen atmosphere. The resulting mixture was stirred for 2 h at 80° C. under nitrogen atmosphere. The resulting mixture was filtered, the filter cake was washed with CH₂Cl₂ (3×2 mL). The filtrate was concentrated under reduced pressure. The residue was purified by silica gel column chromatography, eluted with CH₂Cl₂/MeOH (0% to 10%) to afford followed by reversed-phase flash chromatography with the following conditions: column, C18 silica gel; mobile phase, CH₃CN in Water (10 mmol/L NH₄HCO₃), 10% to 50% gradient in 20 min; detector, 254/220 nm to afford (7R,14R)-1-(difluoromethoxy)-11-(4-hydroxy-4-methylpent-1-yn-1-yl)-6-(methyl-d3)-6,7-dihydro-7,14-methanobenzo[f]benzo[4,5]imidazo[1,2-a][1,4]diazocin-5(14H)-one (31 mg, 38%) as a white solid. MS ESI calculated for C₂₅H₂₀D₃F₂N₃O₃[M+H]⁺, 455.19. found 455.10. ¹H NMR (400 MHz, Chloroform-d) δ 8.49 (d, J=8.3 Hz, 1H), 7.66 (d, J=8.5 Hz, 1H), 7.58 (s, 1H), 7.49-7.42 (m, 1H), 7.38-7.31 (m, 2H), 6.86 (t, J=72.6 Hz, 1H), 6.27 (d, J=6.9 Hz, 1H), 5.12 (d, J=6.7 Hz, 1H), 3.56-3.45 (m, 1H), 2.90 (d, J=13.5 Hz, 1H), 2.63 (s, 2H), 1.39 (s, 6H). ¹⁹F NMR (377 MHz, Chloroform-d) δ −80.29, −80.74, −81.16, −81.60.

### Example 83: (7R,14R)-1-(difluoromethoxy)-11-(5-hydroxypent-1-yn-1-yl)-6-(methyl-d3)-6,7-dihydro-7,14-methanobenzo[f]benzo[4,5]imidazo[1,2-a][1,4]diazocin-5(14H)-one

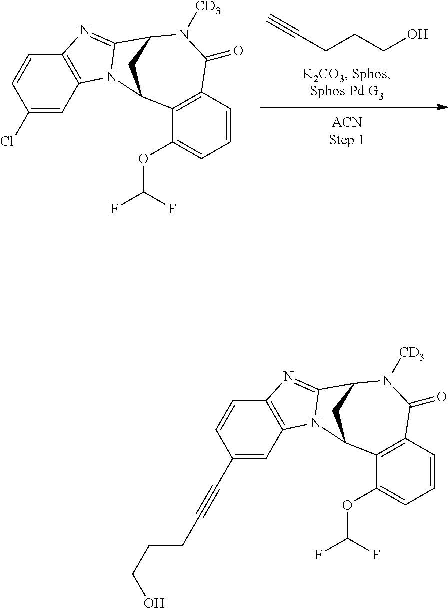

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and pent-4-yn-1-ol (75 mg, 0.890 mmol) in ACN (1 mL) were added K₂CO₃ (123 mg, 0.890 mmol), SPhos Pd Gen.3 (28 mg, 0.036 mmol) and SPhos (15 mg, 0.036 mmol) at room temperature under nitrogen atmosphere. The resulting mixture was stirred for overnight at 80° C. under nitrogen atmosphere. The mixture was concentrated under reduce pressure. The residue was purified by silica gel column chromatography, eluted with CH₂Cl₂/MeOH (12/1) followed by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min; Gradient: 19% B to 36% B in 10 min; Wave Length: 254 nm/220 nm; RT1(min): 13.3) to afford (7R,14R)-1-(difluoromethoxy)-11-(5-hydroxypent-1-yn-1-yl)-6-(methyl-d3)-6,7-dihydro-7,14-methanobenzo[f]benzo[4,5]imidazo[1,2-a][1,4]diazocin-5(14H)-one (24 mg, 31%) as white solid. MS ESI calculated for C₂₄H₁₈D₃F₂N₃O₃[M+H]⁺, 441.17 found 441.20. ¹H NMR (400 MHz, Chloroform-d) δ 8.48 (d, J=8.2 Hz, 1H), 7.62 (d, J=8.4 Hz, 1H), 7.50 (s, 1H), 7.42 (t, J=8.2 Hz, 1H), 7.34 (d, J=8.1 Hz, 1H), 7.29 (d, J=1.5 Hz, 1H), 6.85 (t, J=72.7 Hz, 1H), 6.21 (d, J=7.2 Hz, 1H), 4.98 (d, J=7.1 Hz, 1H), 3.84 (t, J=6.1 Hz, 2H), 3.48-3.41 (m, 1H), 2.86 (d, J=13.6 Hz, 1H), 2.56 (t, J=6.9 Hz, 2H), 1.89 (d, J=6.5 Hz, 2H). ¹⁹F NMR (377 MHz, Chloroform-d) δ −79.96, −80.40, −81.19, −81.63.

### Example 84: (7R,14R)-11-(but-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

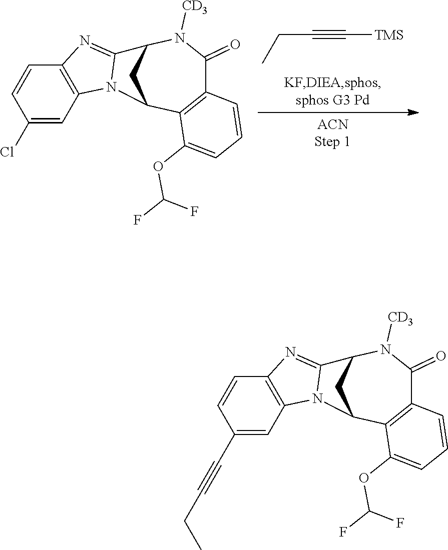

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and but-1-yn-1-yltrimethylsilane (96 mg, 0.765 mmol) in ACN (1 mL) was added KF (15 mg, 0.255 mmol), DIEA (0.2 mL, 1.275 mmol), SPhos (21 mg, 0.051 mmol) and SPhos Pd Gen.3 (40 mg, 0.051 mmol) at room temperature under nitrogen atmosphere. The resulting mixture was stirred for 16 h at 80° C. under nitrogen atmosphere. The mixture was allowed to cool down to room temperature, and concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 60% gradient in 25 min; detector, 254/220 nm to afford (7R,14R)-11-(but-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (10 mg, 9%) as a white solid. MS ESI calculated for C₂₃H₁₆D₃F₂N₃O₂ [M+H]⁺, 411.16. found 411.15. ¹H NMR (400 MHz, Chloroform-d) δ 8.48 (d, J=8.2 Hz, 1H), 7.61 (d, J=8.5 Hz, 1H), 7.49 (s, 1H), 7.42 (t, J=7.2 Hz, 1H), 7.37-7.31 (m, 1H), 7.31-7.27 (m, 1H), 6.84 (t, J=72.7 Hz, 1H), 6.23-6.17 (m, 1H), 5.01-4.90 (m, 1H), 3.51-3.37 (m, 1H), 2.85 (d, J=13.6 Hz, 1H), 2.49-2.39 (m, 2H), 1.25 (t, J=7.5 Hz, 3H). ¹⁹F NMR (377 MHz, Chloroform-d) δ −79.86, −80.30, −81.20, −81.64.

### Example 85: (7R,14R)-11-(cyclopropylethynyl)-1-(difluoromethoxy)-6-(methyl-d3)-6,7-dihydro-7,14-methanobenzo[f]benzo[4,5]imidazo[1,2-a][1,4]diazocin-5(14H)-one

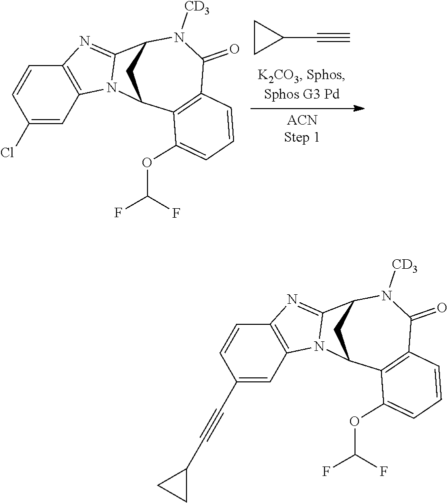

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and ethynylcyclopropane (51 mg, 0.765 mmol) in ACN (1 mL) were added K₂CO₃ (106 mg, 0.765 mmol), SPhos (13 mg, 0.031 mmol) and SPhos Pd Gen.3 (24 mg, 0.031 mmol) at room temperature under nitrogen atmosphere. The resulting mixture was stirred for 2 h at room temperature under nitrogen atmosphere. The resulting mixture was concentrated under vacuum. The residue was purified by silica gel column chromatography, eluted with CH₂Cl₂/MeOH (0% to 10%) to afford followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 32% B to 47% B in 10 min; Wave Length: 254/220 nm) to afford (7R,14R)-11-(cyclopropylethynyl)-1-(difluoromethoxy)-6-(methyl-d3)-6,7-dihydro-7,14-methanobenzo[f]benzo[4,5]imidazo[1,2-a][1,4]diazocin-5(14H)-one (25 mg, 38%) as a light yellow solid. MS ESI calculated for C₂₄H₁₆D₃F₂N₃O₂ [M+H]⁺, 423.16. found 423.00. ¹H NMR (400 MHz, Chloroform-d) δ 8.48 (d, J=8.3, 1.4 Hz, 1H), 7.60 (d, J=8.4 Hz, 1H), 7.48 (s, 1H), 7.45-7.39 (m, 1H), 7.34 (d, J=8.2 Hz, 1H), 7.30-7.26 (m, 1H), 6.84 (t, J=73.3 Hz, 1H), 6.21 (d, J=7.2 Hz, 1H), 4.99 (d, J=7.0 Hz, 1H), 3.50-3.38 (m, 1H), 2.86 (d, J=13.5 Hz, 1H), 1.54-1.40 (m, 1H), 0.93-0.85 (m, 2H), 0.85-0.78 (m, 2H). ¹⁹F NMR (377 MHz, Chloroform-d) δ −79.88, −80.32, −81.21, −81.66.

### Example 86: 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynenitrile

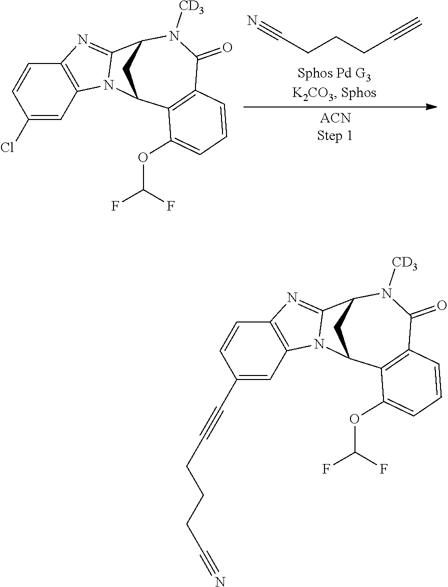

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol, hex-5-ynenitrile (71 mg, 0.765 mmol), SPhos (13 mg, 0.031 mmol), SPhos Pd Gen.3 (24 mg, 0.031 mmol) and K₂CO₃ (105 mg, 0.765 mmol) in ACN (1 mL) was stirred for 2 h at 80° C. under nitrogen atmosphere. The resulting mixture was concentrated under vacuum. The residue was purified by silica gel column chromatography, eluted with MeOH/DCM (0~10%) followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min mL/min; Gradient: 28% B to 43% B in 10 min; Wave Length: 254 nm/220 nm; RT1(min): 11.065) to afford 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynenitrile (33 mg, 48%) as a white solid. MS ESI calculated for C₂₅H₁₇D₃F₂N₄O₂ [M+H]⁺, 450.17. found 450.00. ¹H NMR (400 MHz, Chloroform-d) δ 8.52-8.46 (m, 1H), 7.69-7.62 (m, 1H), 7.56-7.53 (m, 1H), 7.44 (t, J=8.2 Hz, 1H), 7.37-7.29 (m, 2H), 6.87 (t, J=72.7 Hz, 1H), 6.26 (d, J=7.1 Hz, 1H), 5.07 (d, J=7.1 Hz, 1H), 3.54-3.42 (m, 1H), 2.89 (d, J=13.6 Hz, 1H), 2.67-2.54 (m, 4H), 2.04-1.93 (m, 2H). ¹⁹F NMR (377 MHz, Chloroform-d) δ −80.26, −80.71, −81.18, −81.63.

### Example 87: 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynenitrile

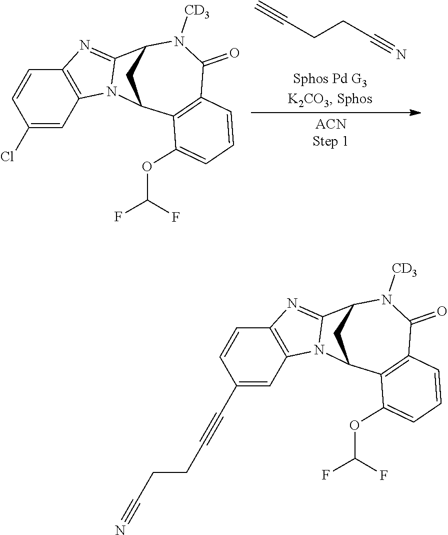

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol), K₂CO₃ (140 mg, 1.020 mmol), SPhos (17 mg, 0.041 mmol) and SPhos Pd Gen.3 (32 mg, 0.041 mmol) in ACN (1 mL) was stirred for 16 h at 80° C. under nitrogen atmosphere. The resulting mixture was concentrated under vacuum. The residue was purified by Prep-HPLC with the following conditions (Column: Xselect CSH C18 OBD Column 30*150 mm 5 μm, n; Mobile Phase A: Water (0.1% FA), Mobile Phase B: MeOH; Flow rate: 60 mL/min; Gradient: 40% B to 60% B in 10 min; Wave Length: 254 nm) to afford 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynenitrile (20 mg, 22%) as a white solid. MS ESI calculated for C₂₄H₁₅D₃F₂N₄O₂[M+H]⁺, 436.16. found 436.05. ¹H NMR (400 MHz, Chloroform-d) δ 8.52-8.45 (m, 1H), 7.65 (d, J=8.5 Hz, 1H), 7.57-7.52 (m, 1H), 7.43 (t, J=8.2 Hz, 1H), 7.38-7.27 (m, 2H), 6.87 (t, J=73.3 Hz, 1H), 6.24 (d, J=7.1 Hz, 1H), 5.01 (d, J=7.1 Hz, 1H), 3.52-3.40 (m, 1H), 2.92-2.84 (m, 1H), 2.84-2.77 (m, 2H), 2.72-2.63 (m, 2H). ¹⁹F NMR (377 MHz, Chloroform-d) δ −80.02, −80.46, −81.35, −81.79.

### Example 88: (7R,14R)-1-(difluoromethoxy)-6-(methyl-d3)-11-(pent-1-yn-1-yl)-6,7-dihydro-7,14-methanobenzo[f]benzo[4,5]imidazo[1,2-a][1,4]diazocin-5(14H)-one

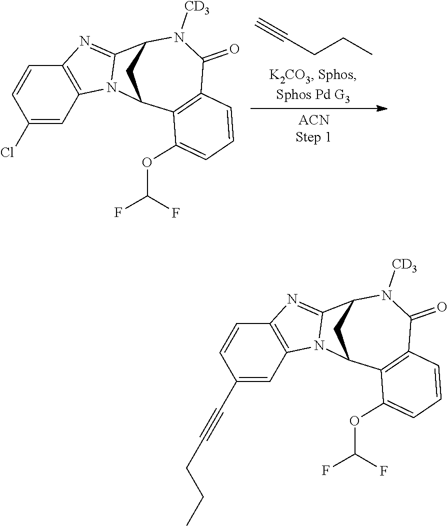

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and K₂CO₃ (106 mg, 0.765 mmol) in CH₃CN (1 mL) were added SPhos (13 mg, 0.031 mmol), SPhos Pd Gen.3 (24 mg, 0.031 mmol) and 1-pentyne (52 mg, 0.764 mmol) at room temperature under nitrogen atmosphere. The resulting mixture was stirred for 16 h at 80° C. under nitrogen atmosphere. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254 nm to afford (7R,14R)-1-(difluoromethoxy)-6-(methyl-d3)-11-(pent-1-yn-1-yl)-6,7-dihydro-7,14-methanobenzo[f]benzo[4,5]imidazo[1,2-a][1,4]diazocin-5(14H)-one (22 mg, 34%) as a white solid. MS ESI calculated for C₂₄H₁₈D₃F₂N₃O₂. [M+H]⁺, 425.18 found 425.15. ¹H NMR (400 MHz, DMSO-d₆) δ 8.30-8.24 (m, 1H), 7.58 (d, J=4.4 Hz, 1H), 7.55 (t, J=74 Hz, 1H), 7.53-7.47 (m, 2H), 7.43 (s, 1H), 7.20-7.15 (m, 1H), 6.21 (d, J=7.1 Hz, 1H), 5.21 (d, J=7.0 Hz, 1H), 3.54-3.46 (m, 1H), 2.79 (d, J=13.7 Hz, 1H), 2.40 (t, J=6.9 Hz, 2H), 1.61-1.49 (m, 2H), 1.01 (t, J=7.3 Hz, 3H). ¹⁹F NMR (377 MHz, DMSO-d₆) δ −82.15.

### Example 89: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-N-(2,2,2-trifluoroethyl)propiolamide

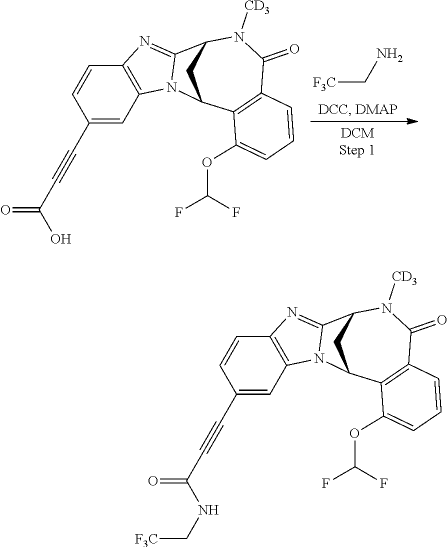 [SMILES: [2H]C([2H])([2H])N1C(=O)c2cccc(OC(F)F)c2[C@H]2C[C@@H]1c1nc3ccc(C#CC(=O)O)cc3n12 ; NCC(F)(F)F ; [2H]C([2H])([2H])N1C(=O)c2cccc(OC(F)F)c2[C@H]2C[C@@H]1c1nc3ccc(C#CC(=O)NCC(F)(F)F)cc3n12]

A solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic acid (50 mg, 0.117 mmol) and 2,2,2-trifluoroethan-1-amine (23 mg, 0.234 mmol) in dry CH₂Cl₂ (1 mL) and cooled to 0° C. DMAP (10 mg, 0.293 mmol) and DCC (29 mg, 0.140 mmol) were added and the mixture was allowed to warm up. The resulting mixture was stirred for 16 h at room temperature. The resulting mixture was filtered, the filter cake was washed with CH₂Cl₂ 3 times. The filtrate was concentrated under reduced pressure. The residue was purified by silica gel column chromatography, eluted with CH₂Cl₂/MeOH (12/1) to afford followed by Prep-HPLC with the following conditions (Column: XBridge Prep Phenyl OBD Column 19*250 mm, 5m; Mobile Phase A: Water (10 mmol/L NH₄HCO₃), Mobile Phase B: ACN; Flow rate: 60 mL/min; Gradient: 27% B to 43% B in 10 min; Wave Length: 254/220 nm) to afford 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-N-(2,2,2-trifluoroethyl)propiolamide (23 mg, 39%) as a white solid. MS ESI calculated for C₂₄H₁₄D₃F₅N₄O₃[M+H]⁺, 508.14. found 508.20. ¹H NMR (400 MHz, Chloroform-d) δ 8.50 (d, J=8.2 Hz, 1H), 7.76-7.53 (m, 2H), 7.52-7.31 (m, 3H), 6.89 (t, J=72.2 Hz, 1H), 6.33-6.31 (m, 2H), 5.27-5.08 (m, 1H), 4.09-3.97 (m, 2H), 3.66-3.48 (m, 1H), 2.92 (d, J=13.1 Hz, 1H). ¹⁹F NMR (377 MHz, Chloroform-d) δ −72.18, −80.25, −80.68, −81.27, −81.72.

### Example 90: (7R,14R)-1-(difluoromethoxy)-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one

Refer to the synthesis of Example 39. MS ESI calculated for C₂₅H₂₂D₃F₂N₃O₂Si [M+H]⁺, 469.19. found 469.05. ¹H NMR (400 MHz, Chloroform-d) δ 8.53-8.44 (m, 1H), 7.63 (d, J=8.5 Hz, 1H), 7.52 (d, J=1.5 Hz, 1H), 7.44 (t, J=8.2 Hz, 1H), 7.36-7.28 (m, 2H), 6.83 (t, J=72.7 Hz, 1H), 6.26 (d, J=7.0 Hz, 1H), 5.11 (d, J=6.8 Hz, 1H), 3.58-3.41 (m, 1H), 2.89 (d, J=13.5 Hz, 1H), 1.72 (s, 2H), 0.17 (s, 9H). ¹⁹F NMR (377 MHz, Chloroform-d) δ −80.11, −80.55, −80.95, −81.39.

### Example 91: (7R,14R)-1-(difluoromethoxy)-11-(hex-1-yn-1-yl)-6-(methyl-d3)-6,7-dihydro-7,14-methanobenzo[f]benzo[4,5]imidazo[1,2-a][1,4]diazocin-5(14H)-one

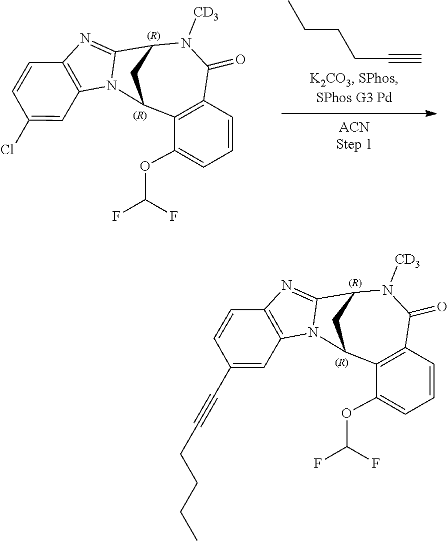

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), K₂CO₃ (106 mg, 0.765 mmol), SPhos (13 mg, 0.031 mmol), SPhos Pd Gen.3 (24 mg, 0.031 mmol and hexyne (63 mg, 0.765 mmol) in ACN (1.5 mL) was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by Prep-HPLC with the following conditions (Column: XBridge Shield RP18 OBD Column 30*150 mm, 5m; Mobile Phase A: Water (10 mmol/L NH₄HCO₃+0.1% NH₃·H₂O), Mobile Phase B: ACN; Flow rate: 60 mL/min; Gradient: 41% B to 60% B in 10 min; Wave Length: 254/220 nm; RT1(min): 10.9) to afford (7R,14R)-1-(difluoromethoxy)-11-(hex-1-yn-1-yl)-6-(methyl-d3)-6,7-dihydro-7,14-methanobenzo[f]benzo[4,5]imidazo[1,2-a][1,4]diazocin-5(14H)-one (33 mg, 49%) as a white solid. MS ESI calculated for C₂₅H₂₀D₃F₂N₃O₂[M+H]⁺, 439.19. found 439.10. ¹H NMR (400 MHz, DMSO-d₆) δ 8.31-8.24 (m, 1H), 7.81-7.55 (m, 2H), 7.53-7.45 (m, 2H), 7.45-7.37 (m, 1H), 7.17 (d, J=8.4 Hz, 1H), 6.21 (d, J=7.1 Hz, 1H), 5.21 (d, J=7.0 Hz, 1H), 3.55-3.40 (m, 1H), 2.79 (d, J=13.8 Hz, 1H), 2.42 (t, J=6.8 Hz, 2H), 1.60-1.39 (m, 4H), 1.02-0.88 (m, 3H). ¹⁹F NMR (377 MHz, DMSO-d₆) δ −82.18.

### Example 92: (7R,14R)-1-(difluoromethoxy)-11-(6-hydroxyhex-1-yn-1-yl)-6-(methyl-d3)-6,7-dihydro-7,14-methanobenzo[f]benzo[4,5]imidazo[1,2-a][1,4]diazocin-5(14H)-one

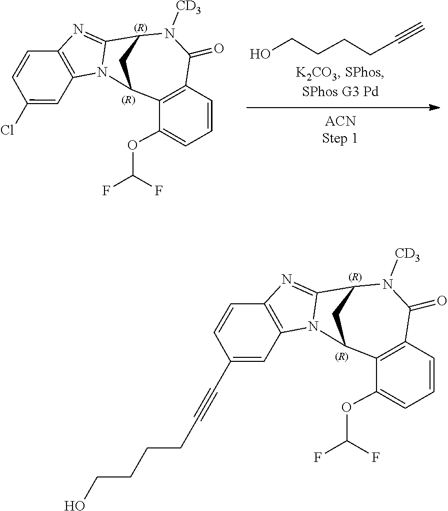

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), K₂CO₃ (106 mg, 0.765 mmol,), SPhos (13 mg, 0.031 mmol), SPhos Pd Gen.3 (24 mg, 0.031 mmol) and hex-5-yn-1-ol (75 mg, 0.765 mmol) in ACN (1.5 mL) was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: MeOH; Flow rate: 60 mL/min; Gradient: 43% B to 61% B in 10 min; Wave Length: 254/220 nm) to afford (7R,14R)-1-(difluoromethoxy)-11-(6-hydroxyhex-1-yn-1-yl)-6-(methyl-d3)-6,7-dihydro-7,14-methanobenzo[f]benzo[4,5]imidazo[1,2-a][1,4]diazocin-5(14H)-one (19 mg, 27%) as a white solid. MS ESI calculated for C₂₅H₂₀D₃F₂N₃O₃[M+H]⁺, 455.19. found 455.10. ¹H NMR (400 MHz, DMSO-d₆) δ 8.34-8.22 (m, 1H), 7.79-7.37 (m, 5H), 7.21-7.14 (m, 1H), 6.21 (d, J=7.1 Hz, 1H), 5.21 (d, J=7.1 Hz, 1H), 4.41 (t, J=5.0 Hz, 1H), 3.55-3.39 (m, 3H), 2.80 (d, J=13.7 Hz, 1H), 2.46-2.39 (m, 2H), 1.63-1.53 (m, 4H). ¹⁹F NMR (377 MHz, DMSO-d₆) δ −81.65, −82.10, −82.12, −82.56.

### Example 93: (7R,14R)-1-(difluoromethoxy)-11-(5-hydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one

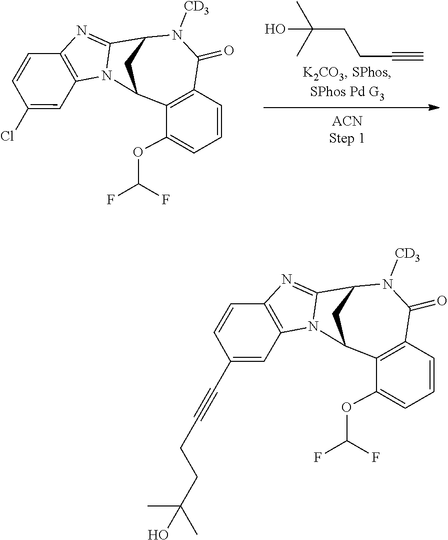

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and K₂CO₃ (106 mg, 0.765 mmol) in ACN (1 mL) were added SPhos (13 mg, 0.031 mmol), SPhos Pd Gen.3 (24 mg, 0.031 mmol) and 2-methylhex-5-yn-2-ol (86 mg, 0.765 mmol) at room temperature. The resulting mixture was stirred for 16 h at 80° C. under nitrogen atmosphere. The resulting mixture was concentrated under vacuum. The residue was purified by silica gel column chromatography, eluted with CH₂Cl₂/MeOH (10/1) followed by Prep-HPLC with the following conditions (Column: XBridge Prep OBD C18 Column 30*150 mm, 5m; Mobile Phase A: Water (10 mmol/L NH₄HCO₃), Mobile Phase B: MeOH; Flow rate: 60 mL/min; Gradient: 46% B to 61% B in 10 min; Wave Length: 254 nm/220 nm nm) to afford (7R,14R)-1-(difluoromethoxy)-11-(5-hydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one (19 mg, 26%) as a white solid. MS ESI calculated for C₂₆H₂₂D₃F₂N₃O₃[M+H]⁺, 469.21 found 469.20. ¹H NMR (400 MHz, DMSO-d₆) δ 8.29-8.24 (m, 1H), 7.78-7.36 (m, 5H), 7.19-7.14 (m, 1H), 6.21 (d, J=7.1 Hz, 1H), 5.21 (d, J=7.1 Hz, 1H), 4.28 (s, 1H), 3.53-3.46 (m, 1H), 2.79 (d, J=13.9 Hz, 1H), 2.47-2.41 (m, 2H), 1.69-1.62 (m, 2H), 1.12 (s, 6H). ¹⁹F NMR (377 MHz, DMSO-d) δ −82.02.

### Example 94: (7R,14R)-1-(difluoromethoxy)-6-(methyl-d3)-11-(5-methylhex-1-yn-1-yl)-6,7-dihydro-7,14-methanobenzo[f]benzo[4,5]imidazo[1,2-a][1,4]diazocin-5(14H)-one

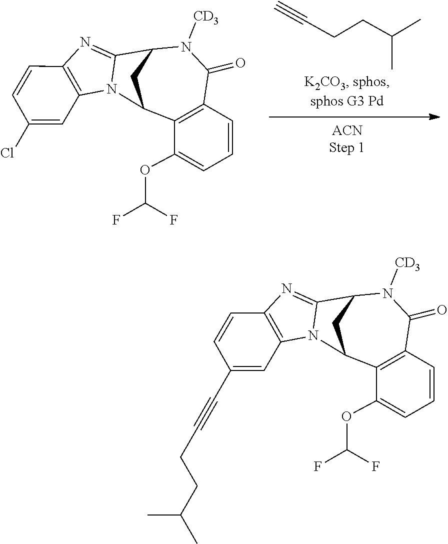

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and 5-methylhex-1-yne (73 mg, 0.765 mmol) in ACN (1 mL) were added K₂CO₃ (106 mg, 0.765 mmol), SPhos (13 mg, 0.031 mmol) and SPhos Pd Gen.3 (24 mg, 0.031 mmol) at room temperature under nitrogen atmosphere. The resulting mixture was stirred for 16 h at 80° C. under nitrogen atmosphere. The resulting mixture was concentrated under vacuum. The residue was purified by silica gel column chromatography, eluted with CH₂Cl₂/MeOH (10/1) followed by Prep-HPLC with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min; Gradient: 47% B to 63% B in 10 min; Wave Length: 254 nm/220 nm nm) to afford (7R,14R)-1-(difluoromethoxy)-6-(methyl-d3)-11-(5-methylhex-1-yn-1-yl)-6,7-dihydro-7,14-methanobenzo[f]benzo[4,5]imidazo[1,2-a][1,4]diazocin-5(14H)-one (27 mg, 39%) as a white solid. MS ESI calculated for C₂₆H₂₂D₃F₂N₃O₂. [M+H]⁺, 453.21 found 453.20. ¹H NMR (400 MHz, DMSO-d₆) δ 8.30-8.24 (m, 1H), 7.59-7.56 (m, 1H), 7.55 (t, J=72 Hz, 1H), 7.53-7.47 (m, 2H), 7.43 (d, J=1.6 Hz, 1H), 7.20-7.14 (m, 1H), 6.21 (d, J=7.1 Hz, 1H), 5.21 (d, J=7.2 Hz, 1H), 3.54-3.43 (m, 1H), 2.79 (d, J=13.8 Hz, 1H), 2.43 (t, J=7.2 Hz, 2H), 1.79-1.69 (m, 1H), 1.49-1.40 (m, 2H), 0.92 (d, J=6.6 Hz, 6H). ¹⁹F NMR (377 MHz, DMSO-d₆) δ −81.71, −82.16, −82.19, −82.63.

### Example 95: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-N-isopropylpropiolamide

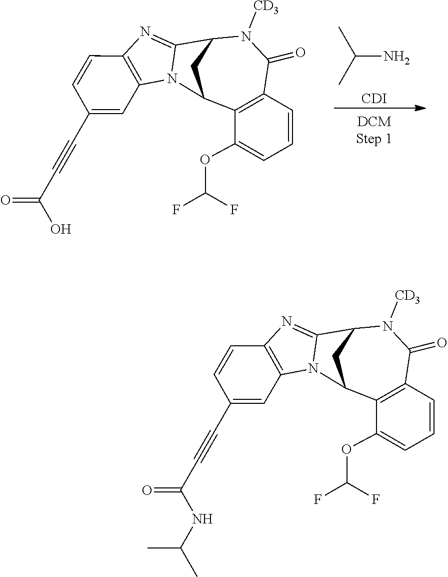

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolic acid (35 mg, 0.082 mmol) in DCM (0.5 mL) was added CDI (20 mg, 0.123 mmol) at room temperature under nitrogen atmosphere. The resulting mixture was stirred for 30 min at room temperature. To the above mixture was added propan-2-amine (15 mg, 0.246 mmol) dropwise at room temperature. The resulting mixture was stirred for additional 2 h at room temperature. The resulting mixture was diluted with CH₂Cl₂ (3 mL) and H₂O (5 mL) The resulting mixture was extracted with CH₂Cl₂ (2×5 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 30% to 60% gradient in 20 min; detector, 254/220 nm to afford 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-N-isopropylpropiolamide (8 mg, 20%) as a white solid. MS ESI calculated for C₂₅H₁₉D₃F₂N₄O₃ [M+H]⁺, 468.18. found 468.25. ¹H NMR (400 MHz, Chloroform-d) δ 8.49 (d, J=8.1 Hz, 1H), 7.72-7.61 (m, 2H), 7.47-7.33 (m, 3H), 6.86 (t, J=72.6 Hz, 1H), 6.23 (d, J=7.0 Hz, 1H), 5.85-5.70 (m, 1H), 5.06-4.93 (m, 1H), 4.27-4.11 (m, 1H), 3.54-3.37 (m, 1H), 2.88 (d, J=13.4 Hz, 1H), 1.32-1.21 (m, 6H). ¹⁹F NMR (377 MHz, Chloroform-d) δ −80.05, −80.49, −81.24, −81.68.

### Example 96: (7R,14R)-1-(difluoromethoxy)-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one

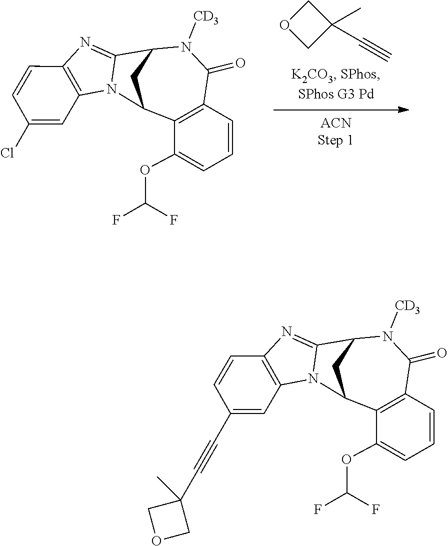

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), K₂CO₃ (106 mg, 0.765 mmol), SPhos (13 mg, 0.031 mmol), SPhos Pd Gen.3 (24 mg, 0.031 mmol) and 3-ethynyl-3-methyloxetane (77 mg, 0.805 mmol) in ACN (1 mL) was stirred for 2 h at 80° C. under nitrogen atmosphere. The resulting mixture was concentrated under reduced pressure. The residue was purified by silica gel column chromatography, eluted with CH₂Cl₂/MeOH (10/1) followed by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 28% B to 43% B in 10 min; Wave Length: 254/220 nm) to afford (7R,14R)-1-(difluoromethoxy)-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one (10 mg, 11%) as a white solid. MS ESI calculated for C₂₅H₁₈D₃F₂N₃O₃[M+H]⁺, 453.17. found 453.00. ¹H NMR (400 MHz, DMSO-d₆) δ 8.31-8.22 (m, 1H), 7.81-7.39 (m, 5H), 7.23 (d, J=8.5 Hz, 1H), 6.22 (d, J=7.1 Hz, 1H), 5.22 (d, J=7.1 Hz, 1H), 4.73 (d, J=5.4 Hz, 2H), 4.44 (d, J=5.4 Hz, 2H), 3.58-3.42 (m, 1H), 2.80 (d, J=13.8 Hz, 1H), 1.64 (s, 3H). ¹⁹F NMR (377 MHz, DMSO-d₆) δ −81.77, −82.21, −82.31, −82.76.

### Example 97: (7R,14R)-1-(difluoromethoxy)-11-((4-hydroxytetrahydro-2H-pyran-4-yl)ethynyl)-6-(methyl-d3)-6,7-dihydro-7,14-methanobenzo[f]benzo[4,5]imidazo[1,2-a][1,4]diazocin-5(14H)-one

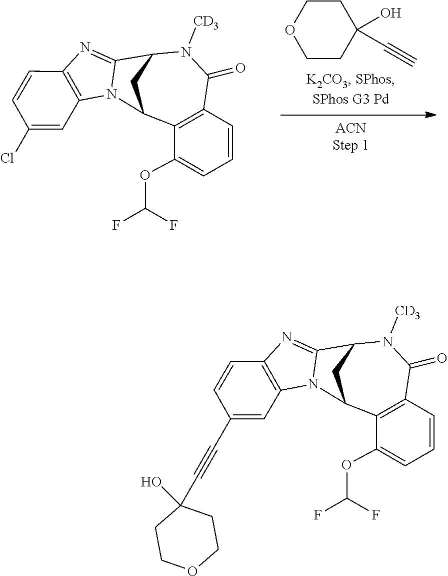

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol), K₂CO₃ (141 mg, 1.020 mmol), SPhos (17 mg, 0.041 mmol), SPhos Pd Gen.3 (32 mg, 0.041 mmol) and 4-ethynyloxan-4-ol (128 mg, 1.020 mmol) in ACN (1 mL) was stirred for 2 h at 80° C. under nitrogen atmosphere. The resulting mixture was concentrated under reduced pressure. The residue was purified by silica gel column chromatography, eluted with CH₂Cl₂/MeOH (10/1) followed by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: MeOH; Flow rate: 60 mL/min; Gradient: 17% B to 37% B in 10 min; Wave Length: 254/220 nm) to afford (7R,14R)-1-(difluoromethoxy)-11-((4-hydroxytetrahydro-2H-pyran-4-yl)ethynyl)-6-(methyl-d3)-6,7-dihydro-7,14-methanobenzo[f]benzo[4,5]imidazo[1,2-a][1,4]diazocin-5(14H)-one (28 mg, 28%) as a white solid. MS ESI calculated for C₂₆H₂₀D₃F₂N₃O₄[M+H]⁺, 483.18. found 483.10. ¹H NMR (400 MHz, DMSO-d₆) δ 8.33-8.21 (m, 1H), 7.81-7.40 (m, 5H), 7.28-7.19 (m, 1H), 6.24 (d, J=7.0 Hz, 1H), 5.73 (s, 1H), 5.22 (d, J=7.1 Hz, 1H), 3.86-3.74 (m, 2H), 3.63-3.44 (m, 3H), 2.81 (d, J=13.8 Hz, 1H), 1.93-1.81 (m, 2H), 1.77-1.65 (m, 2H). ¹⁹F NMR (377 MHz, DMSO-d₆) δ −81.80, −82.25, −82.28, −82.73.

### Example 98: 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoic Acid

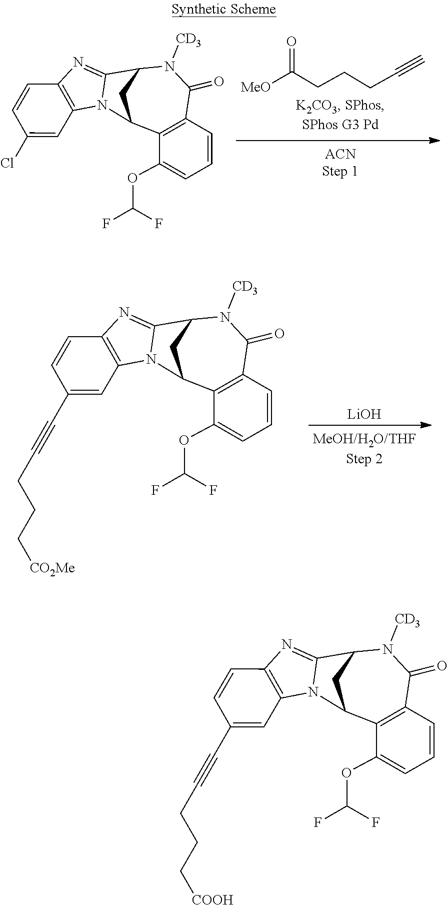

### Preparation 98A: methyl 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoate

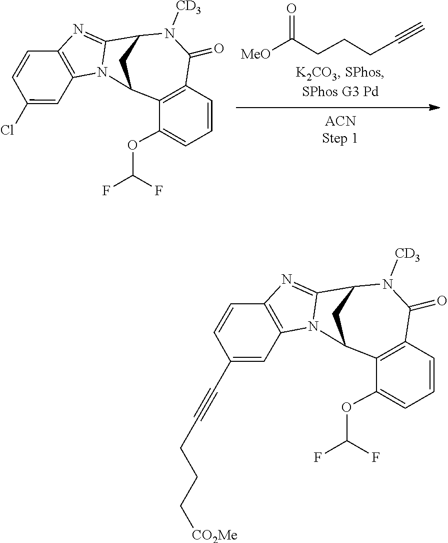

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and K₂CO₃ (106 mg, 0.765 mmol) in ACN (1 mL) were added SPhos (13 mg, 0.031 mmol), SPhos Pd Gen.3 (24 mg, 0.031 mmol) and methyl hex-5-ynoate (94 mg, 0.765 mmol) at room temperature under nitrogen atmosphere. The resulting mixture was stirred for 16 h at 80° C. under nitrogen atmosphere. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254 nm to afford methyl 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoate (20 mg, 27%) as a white solid. MS ESI calculated for C₂₆H₂₀D₃F₂N₃O₄[M+H]⁺, 483.18. found 483.15.

### Example 98: 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoic Acid

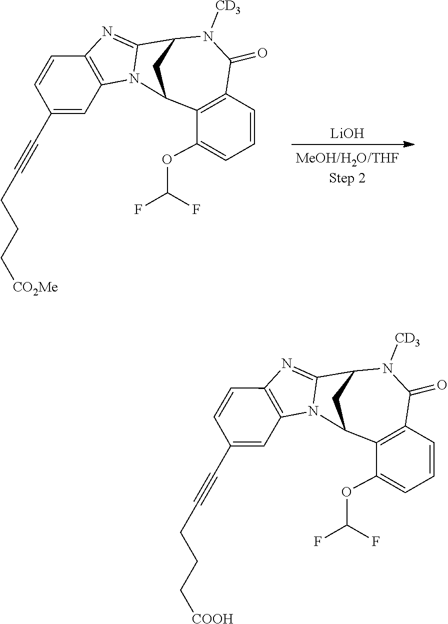 [SMILES: [2H]C([2H])([2H])N1C(=O)c2cccc(OC(F)F)c2[C@H]2C[C@@H]1c1nc3ccc(C#CCCCC(=O)OC)cc3n12 ; "LiOH" ; [2H]C([2H])([2H])N1C(=O)c2cccc(OC(F)F)c2[C@H]2C[C@@H]1c1nc3ccc(C#CCCCC(=O)O)cc3n12]

To a stirred solution of methyl 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoate (20 mg, 0.041 mmol) in MeOH/H₂O/THF (0.3 mL/0.3 mL/0.3 mL) was added LiOH (5 mg, 0.207 mmol) at room temperature. The resulting mixture was stirred for 2 h at room temperature. The resulting mixture was concentrated under vacuum. The resulting mixture was diluted with H₂O (0.3 mL). The mixture was acidified to pH=2 with HCl (2 N). The mixture was purified by reversed-phase flash chromatography with the following conditions: column, C18 silica gel; mobile phase, CH₃CN in Water (0.1% FA), 30% to 50% gradient in 20 min; detector, 254 nm to afford 6-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)hex-5-ynoic acid (9 mg, 43%) as a white solid. MS ESI calculated for C₂₅H₁₈D₃F₂N₃O₄ [M+H]⁺, 469.17. found 469.25. ¹H NMR (400 MHz, DMSO-d₆) δ 12.12 (s, 1H), 8.28-8.22 (m, 1H), 7.78-7.35 (m, 5H), 7.25-7.12 (m, 1H), 6.22 (d, J=7.1 Hz, 1H), 5.21 (d, J=7.1 Hz, 1H), 3.54-3.44 (m, 1H), 2.80 (d, J=13.8 Hz, 1H), 2.46 (t, J=7.1 Hz, 2H), 2.38 (t, J=7.3 Hz, 2H), 1.83-1.70 (m, 2H). ¹⁹F NMR (377 MHz, DMSO-d₆) δ −81.67, −82.12, −82.17, −82.62.

### Example 99: (7R,14R)-1-(difluoromethoxy)-11-(4-hydroxybutyl)-6-(methyl-d3)-6,7-dihydro-7,14-methanobenzo[f]benzo[4,5]imidazo[1,2-a][1,4]diazocin-5(14H)-one

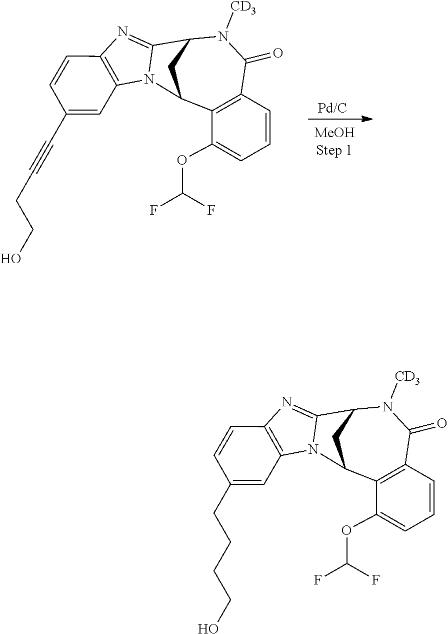 [SMILES: [2H]C([2H])([2H])N1C(=O)c2cccc(OC(F)F)c2[C@H]2C[C@@H]1c1nc3ccc(C#CCCO)cc3n12 ; "Pd/C" ; [2H]C([2H])([2H])N1C(=O)c2cccc(OC(F)F)c2[C@H]2C[C@@H]1c1nc3ccc(CCCCO)cc3n12]

To a stirred solution of (7R,14R)-1-(difluoromethoxy)-11-(4-hydroxybut-1-yn-1-yl)-6-(methyl-d3)-6,7-dihydro-7,14-methanobenzo[f]benzo[4,5]imidazo[1,2-a][1,4]diazocin-5(14H)-one (30 mg, 0.070 mmol) in MeOH (1 mL) was added Pd/C (6 mg) at room temperature under hydrogen atmosphere. The resulting mixture was stirred for 20 h at room temperature under hydrogen atmosphere. The resulting mixture was filtered, the filter cake was washed with MeOH (2×3 mL). The filtrate was concentrated under reduced pressure. The residue was purified by reversed-phase flash chromatography with the following conditions: column, C18 silica gel; mobile phase, CH₃CN in Water (10 mmol/L NH₄HCO₃), 40% to 60% gradient in 20 min; detector, 254 nm to afford (7R,14R)-1-(difluoromethoxy)-11-(4-hydroxybutyl)-6-(methyl-d3)-6,7-dihydro-7,14-methanobenzo[f]benzo[4,5]imidazo[1,2-a][1,4]diazocin-5(14H)-one (18 mg, 58%) as a white solid. MS ESI calculated for C₂₃H₂₀D₃F₂N₃O₃[M+H]⁺, 431.19. found 431.15. ¹H NMR (400 MHz, DMSO-d₆) δ 8.28-8.24 (m, 1H), 7.60 (t, J=72 Hz, 1H), 7.51-7.47 (m, 3H), 7.25 (s, 1H), 7.04-7.00 (m, 1H), 6.19 (d, J=7.1 Hz, 1H), 5.17 (d, J=7.1 Hz, 1H), 4.34 (t, J=5.2 Hz, 1H), 3.52-3.44 (m, 1H), 3.41-3.36 (m, 2H), 2.77 (d, J=13.7 Hz, 1H), 2.63 (t, J=7.5 Hz, 2H), 1.63-1.55 (m, 2H), 1.44-1.37 (m, 2H). ¹⁹F NMR (377 MHz, DMSO-d₆) δ −81.65, −82.10, −82.12, −82.57.

### Example 100: (7R,14R)-1-(difluoromethoxy)-11-((2-(dimethylphosphoryl)pyrimidin-5-yl)ethynyl)-6-(methyl-d3)-6,7-dihydro-7,14-methanobenzo[f]benzo[4,5]imidazo[1,2-a][1,4]diazocin-5(14H)-one

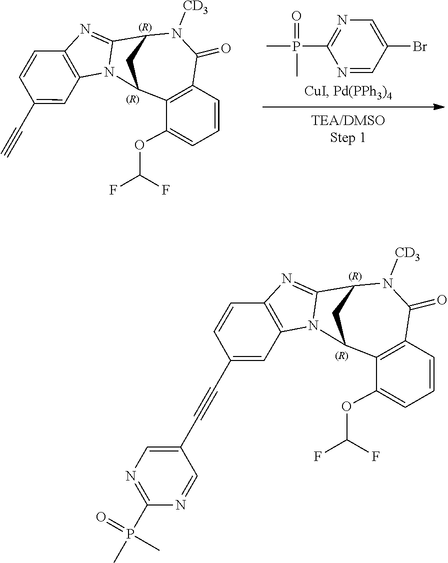

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (100 mg, 0.262 mmol) and 5-bromo-2-(dimethylphosphoryl)pyrimidine (61 mg, 0.262 mmol) in DMSO (2.5 mL) and TEA (2.5 mL) were added CuI (10 mg, 0.052 mmol) and Pd(PPh₃)₄(30 mg, 0.026 mmol) at 25° C. The resulting mixture was stirred for 2 h at 80° C. under nitrogen atmosphere. The resulting mixture was diluted with water (50 mL) and extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (10:1) followed by reversed-phase flash chromatography with the following conditions: column, C18 silica gel; mobile phase, CH₃CN in water (10 mmol/L NH₄HCO₃), 10% to 50% gradient in 10 min; detector, 254 nm. This resulted in (7R,14R)-1-(difluoromethoxy)-11-((2-(dimethylphosphoryl)pyrimidin-5-yl)ethynyl)-6-(methyl-d3)-6,7-dihydro-7,14-methanobenzo[f]benzo[4,5]imidazo[1,2-a][1,4]diazocin-5(14H)-one (59 mg, 42%) as a white solid. MS ESI calculated for C₂₇H₁₉D₃F₂N₅O₃P [M+H]⁺, 537.16. found 537.15. ¹H NMR (400 MHz, Chloroform-d) δ 8.98 (s, 2H), 8.51 (d, J=8.2 Hz, 1H), 7.82-7.67 (m, 2H), 7.55-7.44 (m, 2H), 7.38 (d, J=8.2 Hz, 1H), 6.88 (t, J=72.6 Hz, 1H), 6.36-6.27 (m, 1H), 5.20-5.09 (m, 1H), 3.60-3.47 (m, 1H), 2.94 (d, J=12.5 Hz, 1H), 1.92 (s, 3H), 1.88 (s, 3H). ¹⁹F NMR (376 MHz, Chloroform-d) δ −80.25, −80.70, −80.89, −81.34. ³¹P NMR (162 MHz, Chloroform-d) δ 34.75.

### Example 101: (7R,14R)-1-(difluoromethoxy)-6-(methyl-d3)-11-(4-morpholinobut-1-yn-1-yl)-6,7-dihydro-7,14-methanobenzo[f]benzo[4,5]imidazo[1,2-a][1,4]diazocin-5(14H)-one

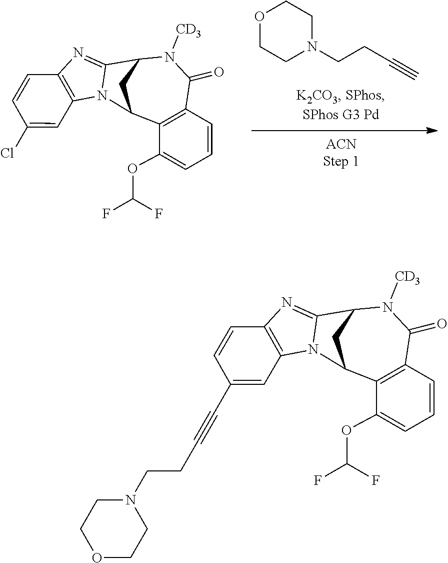

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and 4-(but-3-yn-1-yl)morpholine (106 mg, 0.765 mmol) in ACN (2 mL) were added K₂CO₃ (176 mg, 1.275 mmol), SPhos (21 mg, 0.051 mmol) and SPhos Pd Gen.3 (40 mg, 0.051 mmol) at room temperature under nitrogen atmosphere. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (20/1) followed by reversed-phase flash chromatography with the following conditions: column, C18 silica gel; mobile phase, CH₃CN in Water (10 mmol/L NH₄HCO₃), 30% to 60% gradient in 25 min; detector, 254 nm to afford (7R,14R)-1-(difluoromethoxy)-6-(methyl-d3)-11-(4-morpholinobut-1-yn-1-yl)-6,7-dihydro-7,14-methanobenzo[f]benzo[4,5]imidazo[1,2-a][1,4]diazocin-5(14H)-one (28 mg, 22%) as a white solid. MS ESI calculated for C₂₇H₂₃D₃F₂N₄O₃[M+H]⁺, 496.22. found 496.20. ¹H NMR (400 MHz, DMSO-d₆) δ 8.32-8.21 (m, 1H), 7.80-7.37 (m, 5H), 7.22-7.16 (m, 1H), 6.21 (d, J=7.1 Hz, 1H), 5.21 (d, J=7.1 Hz, 1H), 3.62-3.56 (m, 4H), 3.54-3.45 (m, 1H), 2.80 (d, J=13.8 Hz, 1H), 2.62-2.53 (m, 4H), 2.47-2.41 (m, 4H). ¹⁹F NMR (377 MHz, DMSO-d₆) δ −81.53, −81.98, −81.99, −82.44.

### Example 102: (7R,14R)-1-(difluoromethoxy)-6-(methyl-d3)-11-((tetrahydrofuran-3-yl)ethynyl)-6,7-dihydro-7,14-methanobenzo[f]benzo[4,5]imidazo[1,2-a][1,4]diazocin-5(14H)-one

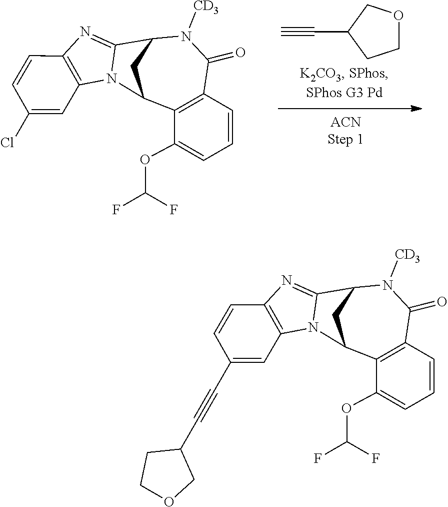

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (50 mg, 0.127 mmol) and 3-ethynyltetrahydrofuran (61 mg, 0.635 mmol) in ACN (1 mL) were added K₂CO₃ (88 mg, 0.635 mmol), SPhos (10 mg, 0.025 mmol) and SPhos Pd Gen.3 (20 mg, 0.025 mmol) at room temperature under nitrogen atmosphere. The resulting mixture was stirred for 2 h at 80° C. under nitrogen atmosphere. The resulting mixture was concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 60% gradient in 25 min; detector, 254/220 nm to afford (7R,14R)-1-(difluoromethoxy)-6-(methyl-d3)-11-((tetrahydrofuran-3-yl)ethynyl)-6,7-dihydro-7,14-methanobenzo[f]benzo[4,5]imidazo[1,2-a][1,4]diazocin-5(14H)-one (12 mg, 21%) as a white solid. MS ESI calculated for C₂₅H₁₈D₃F₂N₃O₃[M+H]⁺, 453.17. found 453.25. ¹H NMR (400 MHz, Chloroform-d) δ 8.48 (d, J=8.2 Hz, 1H), 7.62 (d, J=8.4 Hz, 1H), 7.50 (s, 1H), 7.42 (t, J=8.2 Hz, 1H), 7.34 (d, J=8.1 Hz, 1H), 7.29 (s, 1H), 6.84 (t, J=72.7 Hz, 1H), 6.21 (d, J=7.0 Hz, 1H), 4.96 (d, J=6.8 Hz, 1H), 4.09 (t, J=7.7 Hz, 1H), 4.01-3.94 (m, 1H), 3.93-3.85 (m, 1H), 3.74 (t, J=7.6 Hz, 1H), 3.50-3.38 (m, 1H), 3.27-3.15 (m, 1H), 2.86 (d, J=13.4 Hz, 1H), 2.35-2.24 (m, 1H), 2.15-2.02 (m, 1H). ¹⁹F NMR (377 MHz, Chloroform-d) δ −79.95, −80.40, −81.17, −81.61.

### Example 103: (7R,14R)-1-(difluoromethoxy)-11-(4-(difluoromethoxy)but-1-yn-1-yl)-6-(methyl-d3)-6,7-dihydro-7,14-methanobenzo[f]benzo[4,5]imidazo[1,2-a][1,4]diazocin-5(14H)-one

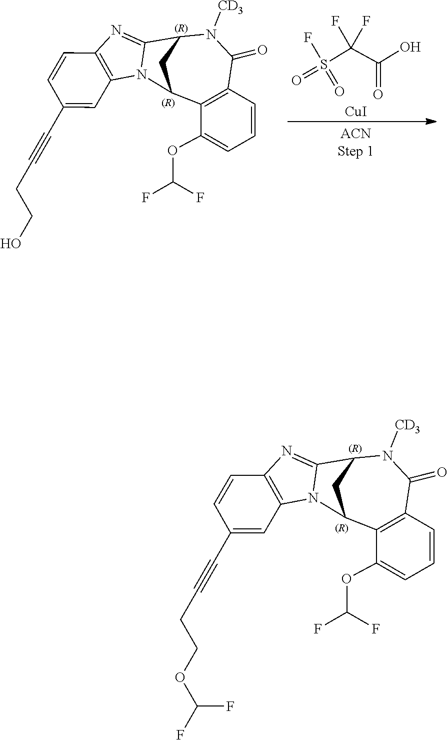

To a stirred solution of (7R,14R)-1-(difluoromethoxy)-11-(4-hydroxybut-1-yn-1-yl)-6-(methyl-d3)-6,7-dihydro-7,14-methanobenzo[f]benzo[4,5]imidazo[1,2-a][1,4]diazocin-5(14H)-one (100 mg, 0.235 mmol) and CuI (9 mg, 0.047 mmol) in ACN (1 mL) was added 2,2-difluoro-2-(fluorosulfonyl)acetic acid (84 mg, 0.470 mmol) at room temperature under nitrogen atmosphere. The resulting mixture was stirred for 2 h at 50° C. under nitrogen atmosphere. The mixture was purified by reversed-phase flash chromatography with the following conditions: column, C18 silica gel; mobile phase, CH₃CN in Water (10 mmol/L NH₄HCO₃), 30% to 50% gradient in 20 min; detector, 254 nm to afford (7R,14R)-1-(difluoromethoxy)-11-(4-(difluoromethoxy)but-1-yn-1-yl)-6-(methyl-d3)-6,7-dihydro-7,14-methanobenzo[f]benzo[4,5]imidazo[1,2-a][1,4]diazocin-5(14H)-one (18 mg, 16%) as a white solid. MS ESI calculated for C₂₄H₁₆D₃F₄N₃O₃ [M+H]⁺, 477.16. found 477.15. ¹H NMR (400 MHz, DMSO-d₆) δ 8.31-8.24 (m, 1H), 7.75-7.36 (m, 5H), 7.21 (d, J=8.4 Hz, 1H), 6.74 (t, J=75.8 Hz, 1H), 6.23 (d, J=7.0 Hz, 1H), 5.23 (d, J=7.0 Hz, 1H), 4.05-3.96 (m, 2H), 3.58-3.45 (m, 2H), 2.87-2.79 (m, 2H). ¹⁹F NMR (377 MHz, DMSO-d₆) δ −82.02, −82.50.

### Example 104: 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoic Acid

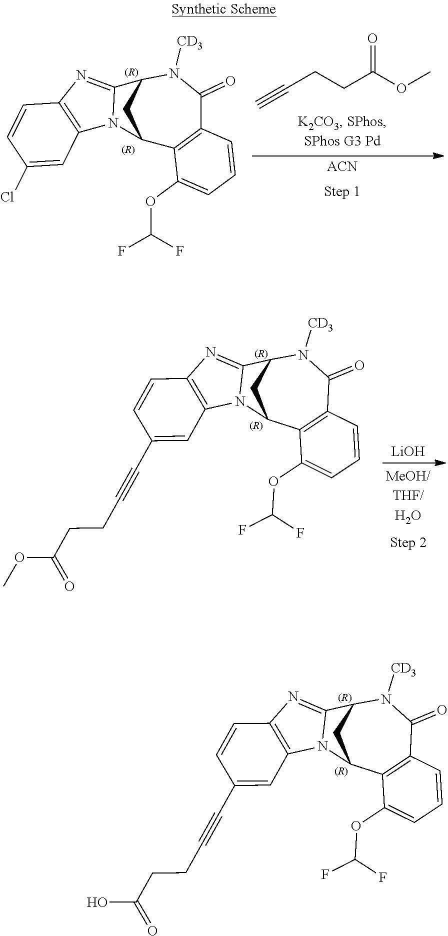

### Preparation 104A: methyl 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoate

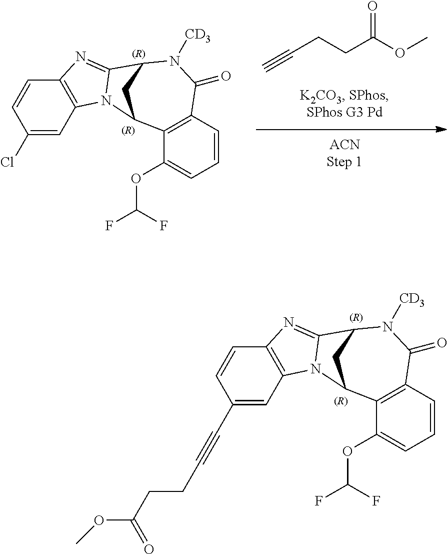

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol), K₂CO₃ (141 mg, 1.020 mmol), SPhos (8 mg, 0.020 mmol), SPhos Pd Gen.3 (16 mg, 0.020 mmol) and methyl pent-4-ynoate (114 mg, 1.020 mmol) in ACN (1 mL) was stirred for 16 h at 80° C. under nitrogen atmosphere. The residue was purified by silica gel column chromatography, eluted with CH₂Cl₂/MeOH (10/1) to afford methyl 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoate (69 mg, 72%) as a light yellow oil. MS ESI calculated for C₂₅H₁₈D₃F₂N₃O₄ [M+H]⁺, 469.17. found 469.20. ¹H NMR (400 MHz, Chloroform-d) δ 8.48 (d, J=8.2 Hz, 1H), 7.61 (d, J=8.4 Hz, 1H), 7.49 (s, 1H), 7.42 (t, J=8.1 Hz, 1H), 7.34 (d, J=8.1 Hz, 1H), 7.28-7.26 (m, 1H), 6.84 (t, J=72.7 Hz, 1H), 6.21 (d, J=7.1 Hz, 1H), 4.95 (d, J=7.0 Hz, 1H), 3.73 (s, 3H), 3.48-3.37 (m, 1H), 2.86 (d, J=13.5 Hz, 1H), 2.78-2.71 (m, 2H), 2.69-2.60 (m, 2H). ¹⁹F NMR (377 MHz, Chloroform-d) δ −79.86, −80.30, −81.12, −81.57.

### Example 104: 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoic Acid

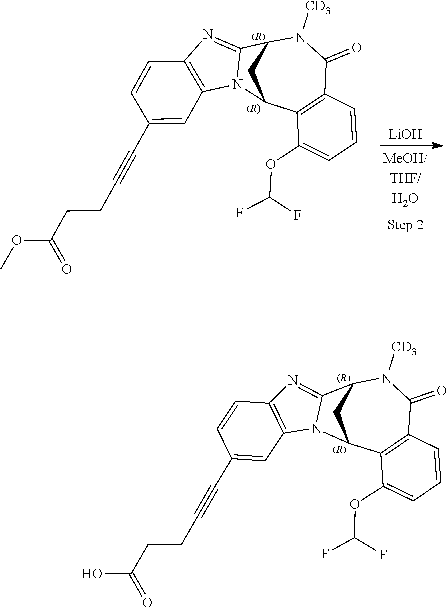

A mixture of methyl 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoate (66 mg, 0.141 mmol) and LiOH (30 mg, 0.705 mmol) in MeOH/THF/H₂O (1/1/1, 1.5 mL) was stirred for 1 h at room temperature. The resulting mixture was concentrated under reduced pressure. The residue was purified by reversed-phase flash chromatography with the following conditions: column, C18 silica gel; mobile phase, CH₃CN in Water (0.1% FA), 30% to 50% gradient in 20 min; detector, 254 nm. This resulted in 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynoic acid (45 mg, 70%) as a white solid. MS ESI calculated for C₂₄H₁₆D₃F₂N₃O₄ [M+H]⁺, 455.15. found 455.20. ¹H NMR (400 MHz, DMSO-d₆) δ 12.28 (s, 1H), 8.32-8.21 (m, 1H), 7.76-7.34 (m, 5H), 7.17 (d, J=8.4 Hz, 1H). 6.21 (d, J=7.1 Hz, 1H), 5.21 (d, J=7.1 Hz, 1H), 3.53-3.44 (m, 1H), 2.80 (d, J=13.8 Hz, 1H), 2.68-2.59 (m, 2H), 2.55-2.45 (m, 2H). ¹⁹F NMR (377 MHz, DMSO-d₆) δ −81.52, −81.97, −82.03, −82.47.

### Example 105: (7R,14R)-11-(3-cyclopropylprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

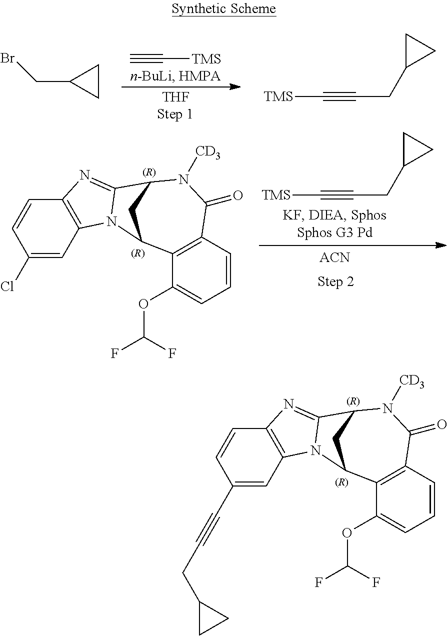

### Preparation 105A: (3-cyclopropylprop-1-yn-1-yl)trimethylsilane

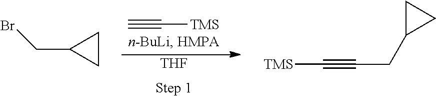

To a stirred mixture of trimethylsilylacetylene (727 mg, 7.407 mmol) in THF (10 mL) was added n-BuLi (3.8 mL, 9.629 mmol, 2.5 M) at −78° C. under nitrogen atmosphere. The resulting mixture was stirred for 1 h at −78° C. under nitrogen atmosphere. To the above mixture was added [bis(dimethylamino)phosphoryl]dimethylamine (1.99 g, 11.111 mmol) at −78° C. The resulting mixture was stirred for additional 30 min at −78° C. To the above mixture was added (bromomethyl)cyclopropane (1.00 g, 7.407 mmol) at −78° C. The resulting mixture was stirred for additional 16 h at room temperature. The reaction was quenched with water at room temperature. The resulting mixture was diluted with Et₂O (20 mL). The resulting mixture was washed with 4×10 mL of water. The organic layer was concentrated under vacuum. The residue was purified by silica gel column chromatography, eluted with PE (100%) to afford (3-cyclopropylprop-1-yn-1-yl)trimethylsilane (130 mg, 11%) as colorless oil. ¹H NMR (400 MHz, Chloroform-d) δ 2.31 (d, J=5.7 Hz, 2H), 0.94-0.88 (m, 1H), 0.48-0.43 (m, 2H), 0.25-0.23 (m, 2H), 0.15 (s, 9H).

### Example 105: (7R,14R)-11-(3-cyclopropylprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

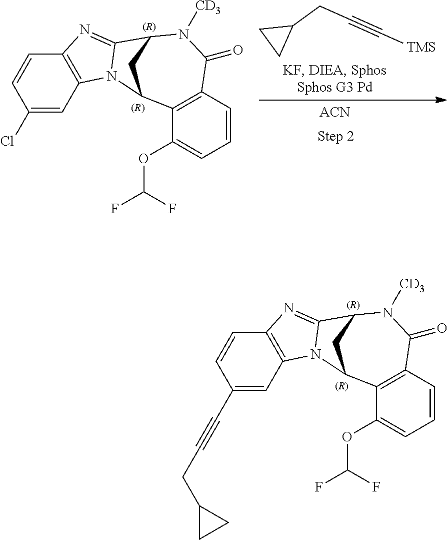

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and (3-cyclopropylprop-1-yn-1-yl)trimethylsilane (81 mg, 0.534 mmol) in ACN (2 mL) were added DIEA (0.2 mL, 0.890 mmol), KF (21 mg, 0.356 mmol), SPhos (15 mg, 0.036 mmol) and SPhos Pd Gen.3 (28 mg, 0.036 mmol) at room temperature under nitrogen atmosphere. The resulting mixture was stirred for 2 h at 80° C. under nitrogen atmosphere. The residue was purified by silica gel column chromatography, eluted with CH₂Cl₂/MeOH (20/1) followed by reversed-phase flash chromatography with the following conditions: column, C18 silica gel; mobile phase, CH₃CN in Water (10 mmol/L NH₄HCO₃), 30% to 60% gradient in 30 min; detector, 254 nm. This resulted in (7R,14R)-11-(3-cyclopropylprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (10 mg, 13%) as a white solid. MS ESI calculated for C₂₅H₁₈D₃F₂N₃O₂[M+H]⁺, 437.18. found 437.15. ¹H NMR (400 MHz, DMSO-d₆) δ 8.32-8.24 (m, 1H), 7.79-7.38 (m, 5H), 7.21-7.15 (m, 1H), 6.22 (d, J=7.1 Hz, 1H), 5.21 (d, J=7.1 Hz, 1H), 3.54-3.46 (m, 1H), 2.80 (d, J=13.8 Hz, 1H), 2.49 (m, 2H), 1.06-0.95 (m, 1H), 0.53-0.45 (m, 2H), 0.34-0.22 (m, 2H). ¹⁹F NMR (377 MHz, DMSO-d₆) δ −82.11.

### Example 106: (7R,14R)-1-(difluoromethoxy)-11-((4-(dimethylphosphoryl)phenyl)ethynyl)-6-(methyl-d3)-6,7-dihydro-7,14-methanobenzo[f]benzo[4,5]imidazo[1,2-a][1,4]diazocin-5(14H)-one

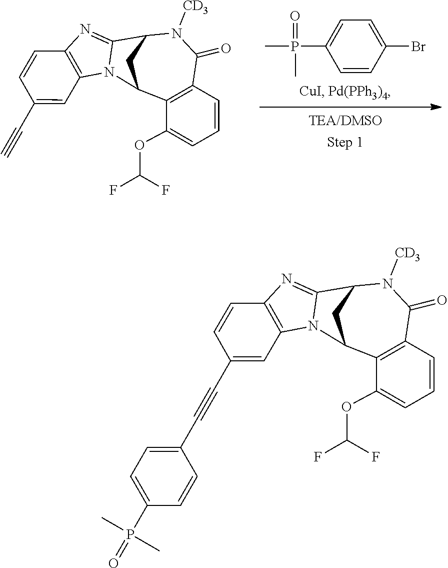

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (70 mg, 0.183 mmol) and (4-bromophenyl)dimethylphosphine oxide (47 mg, 0.201 mmol) in TEA (1 mL) and DMSO (1 mL) were added CuI (7 mg, 0.037 mmol) and Pd(PPh₃)₄(21 mg, 0.018 mmol) at room temperature under nitrogen atmosphere. The resulting mixture was stirred for 2 h at 80° C. under nitrogen atmosphere. The resulting mixture was diluted with water (30 mL). The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 60% gradient in 20 min; detector, 254/220 nm to afford (7R,14R)-1-(difluoromethoxy)-11-((4-(dimethylphosphoryl)phenyl)ethynyl)-6-(methyl-d3)-6,7-dihydro-7,14-methanobenzo[f]benzo[4,5]imidazo[1,2-a][1,4]diazocin-5(14H)-one (49 mg, 50%) as a white solid. MS ESI calculated for C₂₉H₂₁D₃F₂N₃O₃P [M+H]⁺, 535.17. found 535.25. ¹H NMR (400 MHz, Chloroform-d) δ 8.53-8.48 (m, 1H), 7.76-7.70 (m, 3H), 7.68-7.63 (m, 3H), 7.48-7.42 (m, 2H), 7.37 (d, J=8.1 Hz, 1H), 6.87 (t, J=72.7 Hz, 1H), 6.28 (d, J=7.2 Hz, 1H), 5.06 (d, J=7.1 Hz, 1H), 3.55-3.42 (m, 1H), 2.90 (d, J=13.6 Hz, 1H), 1.78 (s, 3H), 1.74 (s, 3H). ¹⁹F NMR (377 MHz, Chloroform-d) δ −80.01, −80.46, −81.07, −81.51. ³¹P NMR (162 MHz, Chloroform-d) δ 33.90.

### Example 107: (7R,14R)-1-(difluoromethoxy)-11-((5-(dimethylphosphoryl)thiophen-2-yl)ethynyl)-6-(methyl-d3)-6,7-dihydro-7,14-methanobenzo[f]benzo[4,5]imidazo[1,2-a][1,4]diazocin-5(14H)-one

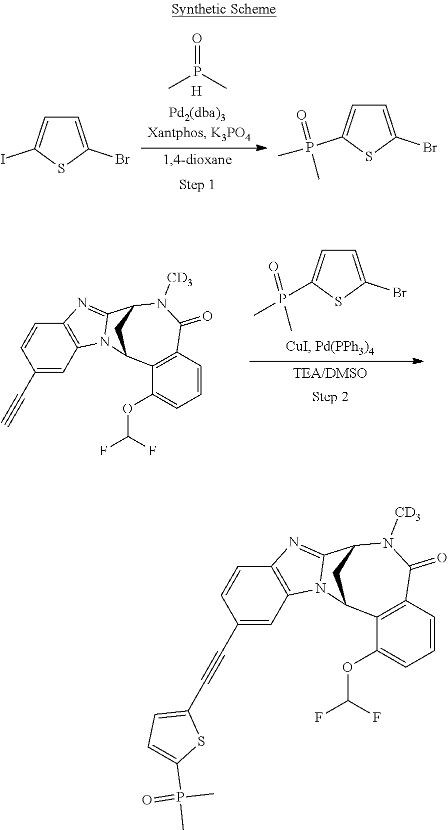

### Preparation 107A: (5-bromothiophen-2-yl)dimethylphosphine Oxide

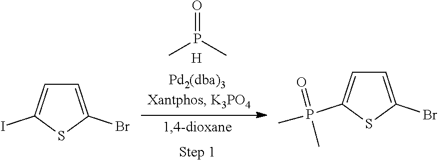

A mixture of 2-bromo-5-iodothiophene (800 mg, 2.769 mmol), dimethylphosphine oxide (238 mg, 3.046 mmol), Pd₂(dba)₃ (127 mg, 0.138 mmol), XantPhos (160 mg, 0.277 mmol) and K₃PO₄ (882 mg, 4.154 mmol) in 1,4-dioxane (8 mL) at room temperature under nitrogen atmosphere. The resulting mixture was stirred for 16 h at 90° C. under nitrogen atmosphere. The resulting mixture was concentrated under vacuum. The residue was purified by silica gel column chromatography, eluted with CH₂Cl₂/MeOH (0% to 10%) to afford (5-bromothiophen-2-yl)dimethylphosphine oxide (350 mg, 52%) as a yellow solid. MS ESI calculated for C₆H₈BrOPS [M+H]⁺, 238.92 240.92. found 238.80 240.80. ¹H NMR (400 MHz, Chloroform-d) δ 7.34-7.29 (m, 1H), 7.16-7.13 (m, 1H), 1.81 (s, 3H), 1.77 (s, 3H). ³¹P NMR (162 MHz, Chloroform-d) δ 28.88.

### Example 107: (7R,14R)-1-(difluoromethoxy)-11-((5-(dimethylphosphoryl)thiophen-2-yl)ethynyl)-6-(methyl-d3)-6,7-dihydro-7,14-methanobenzo[f]benzo[4,5]imidazo[1,2-a][1,4]diazocin-5(14H)-one

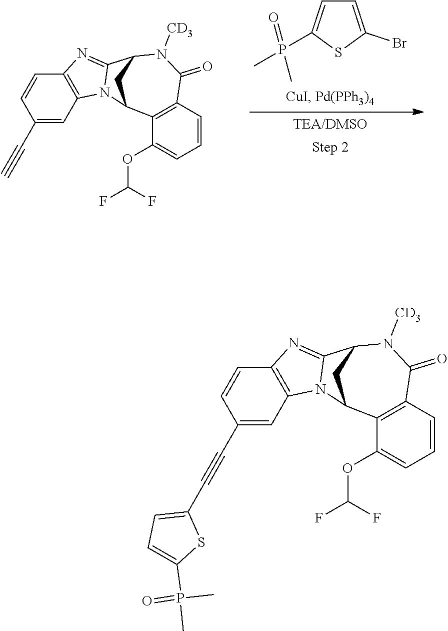

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (70 mg, 0.183 mmol) and (5-bromothiophen-2-yl)dimethylphosphine oxide (53 mg, 0.220 mmol) in TEA (1 mL) and DMSO (1 mL) were added CuI (7 mg, 0.037 mmol) and Pd(PPh₃)₄(21 mg, 0.018 mmol) at room temperature under nitrogen atmosphere. The resulting mixture was stirred for 2 h at 80° C. under nitrogen atmosphere. The resulting mixture was diluted with water (30 mL). The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 50% gradient in 20 min; detector, 254/220 nm to afford (7R,14R)-1-(difluoromethoxy)-11-((5-(dimethylphosphoryl)thiophen-2-yl)ethynyl)-6-(methyl-d3)-6,7-dihydro-7,14-methanobenzo[f]benzo[4,5]imidazo[1,2-a][1,4]diazocin-5(14H)-one (46 mg, 46%) as a white solid. MS ESI calculated for C₂₇H₁₉D₃F₂N₃O₃PS [M+H]⁺, 541.13. found 541.15. ¹H NMR (400 MHz, Chloroform-d) δ 8.53-8.46 (m, 1H), 7.71 (d, J=8.4 Hz, 1H), 7.64 (s, 1H), 7.48-7.40 (m, 3H), 7.37 (d, J=8.1 Hz, 1H), 7.33-7.31 (m, 1H), 6.87 (t, J=73.2, 1H), 6.27 (d, J=7.0 Hz, 1H), 5.05 (d, J=7.0 Hz, 1H), 3.56-3.41 (m, 1H), 2.90 (d, J=13.6 Hz, 1H), 1.84 (s, 3H), 1.81 (s, 3H). ¹⁹F NMR (377 MHz, Chloroform-d) δ −80.03, −80.47, −81.11, −81.56. ³¹P NMR (162 MHz, Chloroform-d) δ 28.76.

### Example 108: (7R,14R)-1-(difluoromethoxy)-6-(methyl-d3)-11-(4,4,4-trifluorobut-1-yn-1-yl)-6,7-dihydro-7,14-methanobenzo[f]benzo[4,5]imidazo[1,2-a][1,4]diazocin-5(14H)-one

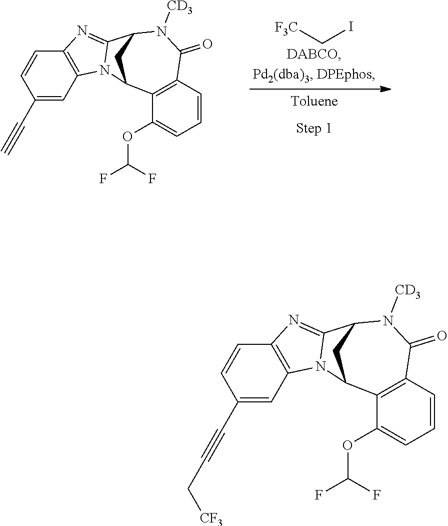 [SMILES: [2H]C([2H])([2H])N1C(=O)c2cccc(OC(F)F)c2[C@H]2C[C@@H]1c1nc3ccc(C#C)cc3n12 ; FC(F)(F)CI ; [2H]C([2H])([2H])N1C(=O)c2cccc(OC(F)F)c2[C@H]2C[C@@H]1c1nc3ccc(C#CCC(F)(F)F)cc3n12]

To a solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (60 mg, 0.157 mmol) and 1,1,1-trifluoro-2-iodoethane (65 mg, 0.314 mmol) in Toluene (2 mL) were added 1,4-diazabicyclo[2.2.2]octane (35 mg, 0.314 mmol), Pd₂(dba)₃ (14 mg, 0.016 mmol) and {2-[2-(diphenylphosphanyl)phenoxy]phenyl}diphenylphosphane (16 mg, 0.031 mmol). After stirring for overnight at 80° C. under nitrogen atmosphere, the resulting mixture was concentrated under reduced pressure. The residue was purified by silica gel column chromatography, eluted with DCM/MeOH (0% to 6%) followed by Prep-HPLC with the following conditions: Column: XBridge Prep OBD C18 Column 30*150 mm, 5m; Mobile Phase A: Water (10 mmol/L NH₄HCO₃+0.1% NH₃·H₂O), Mobile Phase B: ACN; Flow rate: 60 mL/min mL/min; Gradient: 34% B to 52% B in 10 min; Wave Length: 254 nm/220 nm to afford (7R,14R)-1-(difluoromethoxy)-6-(methyl-d3)-11-(4,4,4-trifluorobut-1-yn-1-yl)-6,7-dihydro-7,14-methanobenzo[f]benzo[4,5]imidazo[1,2-a][1,4]diazocin-5(14H)-one (40 mg, 54%) as a white solid. MS ESI calculated for C₂₃H₁₃D₃F₅N₃O₂[M+H]⁺, 465.14. found 465.15. ¹H NMR (400 MHz, Chloroform-d) δ 8.52-8.45 (m, 1H), 7.66 (d, J=8.5 Hz, 1H), 7.58 (d, J=1.5 Hz, 1H), 7.44 (t, J=8.2 Hz, 1H), 7.37-7.30 (m, 2H), 7.04-6.65 (m, 1H), 6.25 (d, J=7.1 Hz, 1H), 5.03 (d, J=6.9 Hz, 1H), 3.54-3.40 (m, 1H), 3.29 (q, J=9.5 Hz, 2H), 2.88 (d, J=13.5 Hz, 1H). ¹⁹F NMR (377 MHz, Chloroform-d) δ −66.42, −80.09, −80.54, −81.30, −81.75.

### Example 109: (7R,14R)-1-(difluoromethoxy)-9-fluoro-6-(methyl-d3)-11-(prop-1-yn-1-yl)-6,7-dihydro-7,14-methanobenzo[f]benzo[4,5]imidazo[1,2-a][1,4]diazocin-5(14H)-one

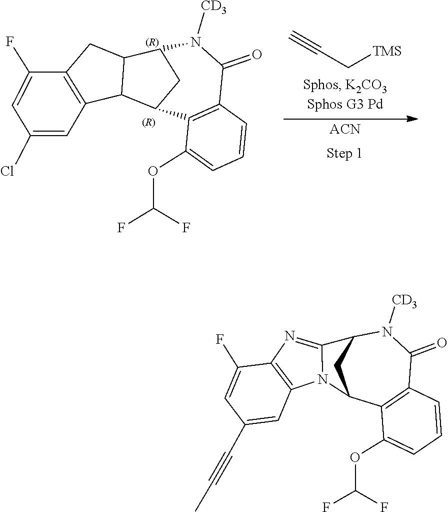

To a solution of (7R,14R)-11-chloro-1-(difluoromethoxy)-9-fluoro-6-(methyl-d3)-6,7-dihydro-7,14-methanobenzo[f]benzo[4,5]imidazo[1,2-a][1,4]diazocin-5(14H)-one (60 mg, 0.146 mmol) and trimethyl(prop-2-yn-1-yl)silane (81 mg, 0.730 mmol) in ACN (1 mL) were added SPhos (12 mg, 0.029 mmol), SPhos Pd Gen.3 (11 mg, 0.015 mmol) and K₂CO₃ (100 mg, 0.730 mmol). After stirring for overnight at 80° C. under a nitrogen atmosphere, the resulting mixture was concentrated under reduced pressure. The residue was purified by silica gel column chromatography, eluted with DCM/MeOH (0% to 6%) followed by Prep-HPLC with the following conditions: Column: XBridge Prep OBD C18 Column 30*150 mm, 5m; Mobile Phase A: Water (10 mmol/L NH₄HCO₃+0.1% NH₃·H₂O), Mobile Phase B: ACN; Flow rate: 60 mL/min mL/min; Gradient: 34% B to 52% B in 10 min; Wave Length: 254 nm to afford (7R,14R)-1-(difluoromethoxy)-9-fluoro-6-(methyl-d3)-11-(prop-1-yn-1-yl)-6,7-dihydro-7,14-methanobenzo[f]benzo[4,5]imidazo[1,2-a][1,4]diazocin-5(14H)-one (15 mg, 24%) as a white solid. MS ESI calculated for C22H₁₃D₃F₃N₃O₂ [M+H]⁺, 415.14 found 415.15. ¹H NMR (400 MHz, Chloroform-d) δ 8.54-8.41 (m, 1H), 7.43 (t, J=8.2 Hz, 1H), 7.34 (d, J=8.0 Hz, 1H), 7.29-7.27 (m, 1H), 7.03-6.94 (m, 1H), 7.00-6.63 (m, 1H), 6.20 (d, J=7.2 Hz, 1H), 4.96 (d, J=7.0 Hz, 1H), 3.53-3.39 (m, 1H), 2.86 (d, J=13.5 Hz, 1H), 2.06 (s, 3H). ¹⁹F NMR (377 MHz, Chloroform-d) δ −79.92, −80.36, −81.07, −81.51, −127.06.

### Example 110: (7R,14R)-1-(difluoromethoxy)-11-((3-hydroxycyclobutyl)ethynyl)-6-(methyl-d3)-6,7-dihydro-7,14-methanobenzo[f]benzo[4,5]imidazo[1,2-a][1,4]diazocin-5(14H)-one

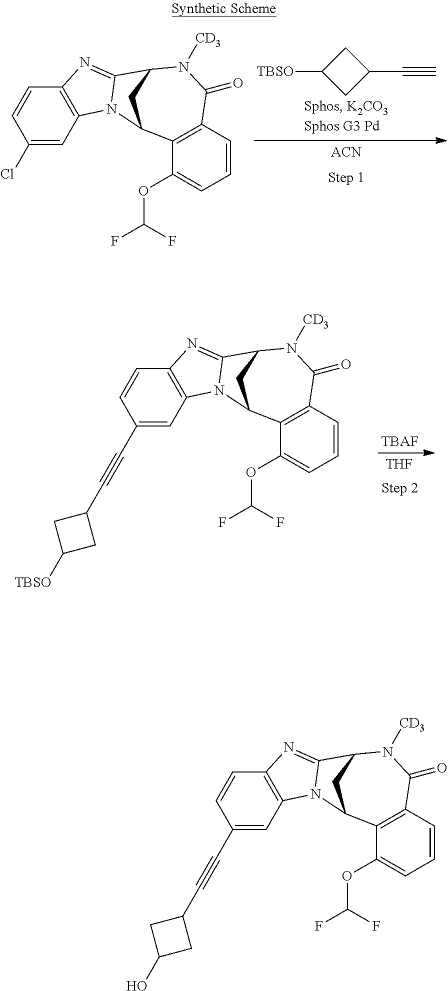

### Preparation 110A: (7R,14R)-11-((3-((tert-butyldimethylsilyl)oxy)cyclobutyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

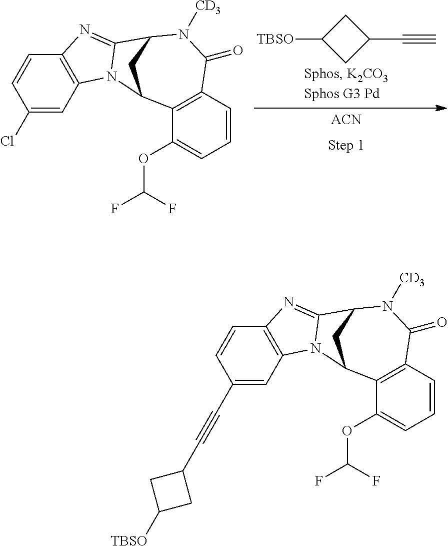 [SMILES: [2H]C([2H])([2H])N1C(=O)c2cccc(OC(F)F)c2[C@H]2C[C@@H]1c1nc3ccc(Cl)cc3n12 ; C#CC1CC(O[Si](C)(C)C(C)(C)C)C1 ; "ACN" ; [2H]C([2H])([2H])N1C(=O)c2cccc(OC(F)F)c2[C@H]2C[C@@H]1c1nc3ccc(C#CC4CC(O[Si](C)(C)C(C)(C)C)C4)cc3n12]

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (250 mg, 0.636 mmol) and tert-butyl(3-ethynylcyclobutoxy)dimethylsilane (201 mg, 0.954 mmol) in ACN (5 mL) were added K₂CO₃ (440 mg, 3.180 mmol), SPhos (52 mg, 0.127 mmol) and SPhos Pd Gen.3 (99 mg, 0.127 mmol) at room temperature. The resulting mixture was stirred for 16 h at 80° C. under nitrogen atmosphere. The resulting mixture was concentrated under vacuum. The residue was purified by silica gel column chromatography, eluted with CH₂Cl₂/MeOH (20/1) to afford (7R,14R)-11-((3-((tert-butyldimethylsilyl)oxy)cyclobutyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (300 mg) as a yellow solid. MS ESI calculated for C₃₁H₃₂D₃F₂N₃O₃Si [M+H]⁺, 567.26. found 567.30.

### Example 110: (7R,14R)-1-(difluoromethoxy)-11-((3-hydroxycyclobutyl)ethynyl)-6-(methyl-d3)-6,7-dihydro-7,14-methanobenzo[f]benzo[4,5]imidazo[1,2-a][1,4]diazocin-5(14H)-one

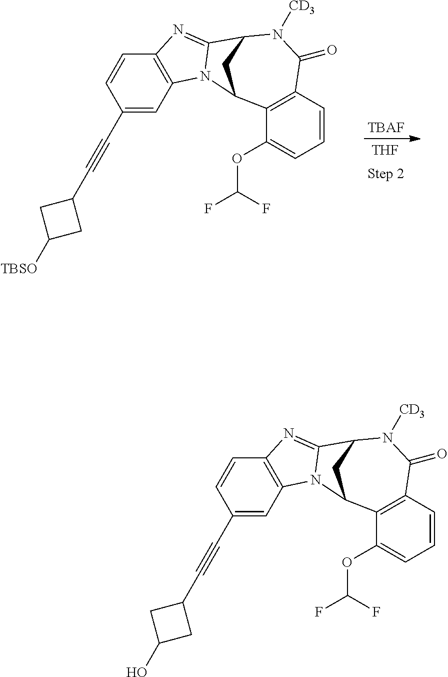

A mixture of (7R,14R)-11-((3-((tert-butyldimethylsilyl)oxy)cyclobutyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (30 mg, 0.053 mmol) and TBAF (17 mg, 0.064 mmol) in THF (1 mL) was stirred for 2 h at room temperature. The resulting mixture was diluted with DCM (20 mL). The residue was washed with water (3×20 mL). The organic layer was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 25% to 55% gradient in 25 min; detector, 254 nm. This resulted in (7R,14R)-1-(difluoromethoxy)-11-((3-hydroxycyclobutyl)ethynyl)-6-(methyl-d3)-6,7-dihydro-7,14-methanobenzo[f]benzo[4,5]imidazo[1,2-a][1,4]diazocin-5(14H)-one (6 mg, 25%) as a white solid. MS ESI calculated for C₂₅H₁₈D₃F₂N₃O₃ [M+H]⁺, 453.17. found 453.15. ¹H NMR (400 MHz, DMSO-d₆) δ 8.33-8.22 (m, 1H), 7.82-7.39 (m, 5H), 7.23-7.11 (m, 1H), 6.26-6.16 (m, 1H), 5.27-5.16 (m, 2H), 4.47-3.91 (m, 1H), 3.56-3.44 (m, 1H), 3.21-3.10 (m, 1H), 2.80 (d, J=13.7 Hz, 1H), 2.64-2.56 (m, 1H), 2.36-2.26 (m, 1H), 2.26-2.13 (m, 1H), 1.98-1.90 (m, 1H). ¹⁹F NMR (377 MHz, DMSO-d₆) δ −81.74, −82.05, −82.19, −82.68.

### Example 111: (7R,14R)-1-(difluoromethoxy)-11-(4-hydroxybut-1-yn-1-yl)-6-(methyl-d3)-6,7-dihydro-7,14-methanobenzo[f]benzo[4,5]imidazo[1,2-a][1,4]diazocin-5(14H)-one

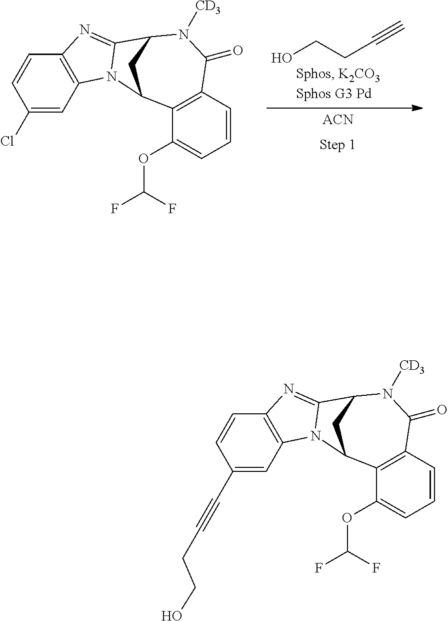

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (500 mg, 1.273 mmol) and K₂CO₃ (880 mg, 6.365 mmol) in ACN (10 mL) were added SPhos (105 mg, 0.255 mmol), SPhos Pd Gen.3 (199 mg, 0.255 mmol) and 3-butyn-1-ol (446 mg, 6.365 mmol) at room temperature under nitrogen atmosphere. The resulting mixture was stirred for 16 h at 80° C. under nitrogen atmosphere. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254 nm to afford (7R,14R)-1-(difluoromethoxy)-11-(4-hydroxybut-1-yn-1-yl)-6-(methyl-d3)-6,7-dihydro-7,14-methanobenzo[f]benzo[4,5]imidazo[1,2-a][1,4]diazocin-5(14H)-one (258 mg, 48%) as a white solid. MS ESI calculated for C₂₃H₁₆D₃F₂N₃O₃ [M+H]⁺, 427.16. found 427.15. ¹H NMR (400 MHz, DMSO-d₆) δ 8.29-8.24 (m, 1H), 7.57 (d, J=3.7 Hz, 1H), 7.56 (t, J=72 Hz, 1H), 7.54-7.45 (m, 2H), 7.41 (s, 1H), 7.21-7.17 (m, 1H), 6.21 (d, J=7.2 Hz, 1H), 5.21 (d, J=7.1 Hz, 1H), 4.88 (t, J=5.6 Hz, 1H), 3.61-3.54 (m, 2H), 3.54-3.46 (m, 1H), 2.79 (d, J=13.7 Hz, 1H), 2.56 (d, J=6.9 Hz, 2H). ¹⁹F NMR (377 MHz, DMSO-d₆) δ −81.50, −81.95, −82.00, −82.45.

### Example 112: (7R,14R)-1-(difluoromethoxy)-11-((6-(dimethylphosphoryl)pyridin-3-yl)ethynyl)-6-(methyl-d3)-6,7-dihydro-7,14-methanobenzo[f]benzo[4,5]imidazo[1,2-a][1,4]diazocin-5(14H)-one

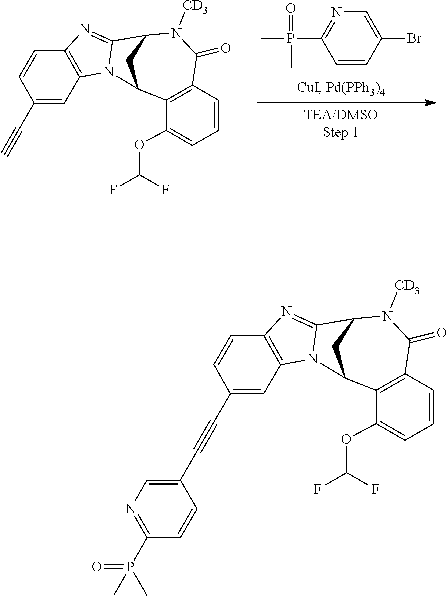

To a stirred solution of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (70 mg, 0.183 mmol) and (5-bromopyridin-2-yl)dimethylphosphine oxide (56 mg, 0.238 mmol) in TEA (1 mL) and DMSO (1 mL) were added CuI (7 mg, 0.037 mmol) and Pd(PPh₃)₄(21 mg, 0.018 mmol) at room temperature under nitrogen atmosphere. The resulting mixture was stirred for 2 h at 80° C. under nitrogen atmosphere. The resulting mixture was diluted with water (30 mL). The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (10/1) followed by Prep-HPLC with the following conditions (Column: YMC-Actus Triart C18 ExRS 30*150 mm, 5 μm; Mobile Phase A: Water (10 mmol/L NH₄HCO₃+0.05% NH₃·H₂O), Mobile Phase B: ACN; Flow rate: 60 mL/min; Gradient: 20% B to 37% B in 10 min; Wave Length: 254/220 nm) to afford (7R,14R)-1-(difluoromethoxy)-11-((6-(dimethylphosphoryl)pyridin-3-yl)ethynyl)-6-(methyl-d3)-6,7-dihydro-7,14-methanobenzo[f]benzo[4,5]imidazo[1,2-a][1,4]diazocin-5(14H)-one (42 mg, 43%) as a white solid. MS ESI calculated for C₂₈H₂₀D₃F₂N₄O₃P [M+H]⁺, 536.17. found 536.20. ¹H NMR (400 MHz, Chloroform-d) δ 8.84 (s, 1H), 8.54-8.48 (m, 1H), 8.16-8.07 (m, 1H), 8.00-7.93 (m, 1H), 7.78-7.73 (m, 1H), 7.70 (s, 1H), 7.51-7.42 (m, 2H), 7.37 (d, J=8.2 Hz, 1H), 6.87 (t, J=72.7, 1H), 6.30 (d, J=6.8 Hz, 1H), 5.10 (d, J=6.7 Hz, 1H), 3.57-3.47 (m, 1H), 2.92 (d, J=13.4 Hz, 1H), 1.82 (s, 3H), 1.78 (s, 3H). ¹⁹F NMR (377 MHz, Chloroform-d) δ −80.12, −80.57, −80.93, −81.37. ³¹P NMR (162 MHz, Chloroform-d) δ 36.91.

### Example 113: (7R,14R)-11-((3-aminocyclobutyl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

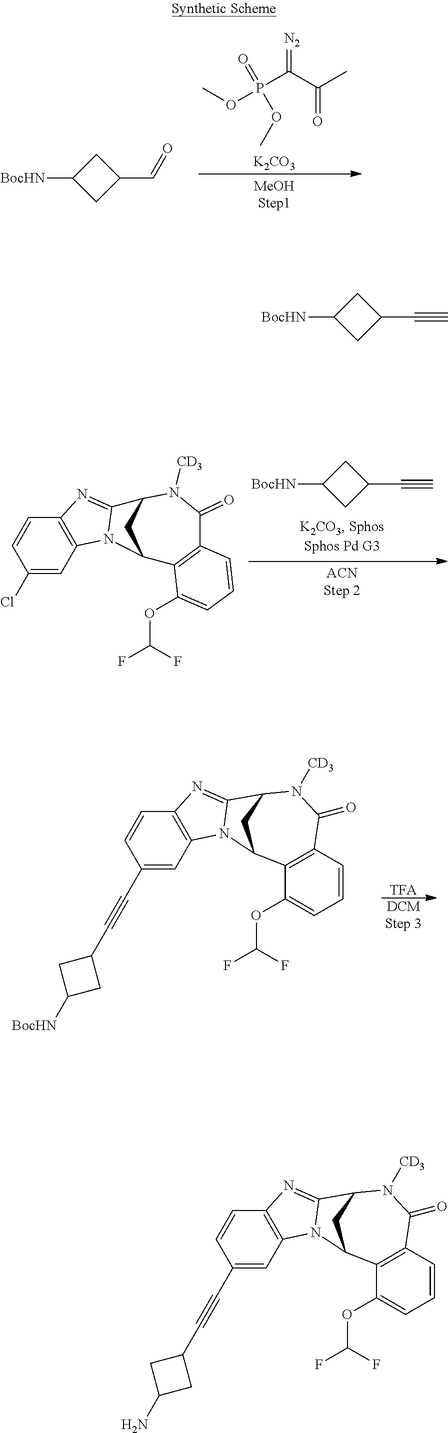

### Preparation 113A: tert-butyl N-(3-ethynylcyclobutyl)carbamate

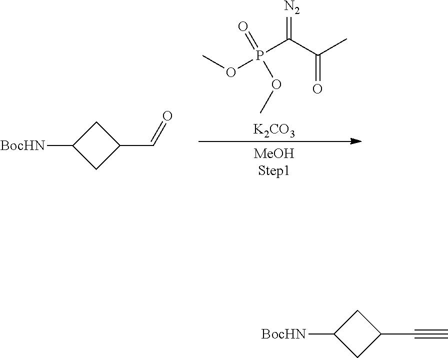

A solution of tert-butyl N-(3-formylcyclobutyl)carbamate (450 mg, 2.258 mmol) in MeOH (2 mL) was treated with K₂CO₃ (624 mg, 4.516 mmol) for 10 min at room temperature under nitrogen atmosphere followed by the addition of seyferth-gilbert homologation (651 mg, 3.387 mmol) dropwise at 0° C. The resulting mixture was stirred for 3 h at room temperature. The resulting mixture was concentrated under reduced pressure. The residue was purified by silica gel column chromatography, eluted with PE/EA (10/1) to afford tert-butyl N-(3-ethynylcyclobutyl)carbamate (295 mg, 67%) as a white solid. MS ESI calculated for C₁₁H₁₇NO₂ found N/A. ¹H NMR (400 MHz, Chloroform-d) δ 4.88-3.80 (m, 2H), 3.00-2.39 (m, 3H), 2.22-2.08 (m, 1H), 2.06-1.88 (m, 1H), 1.44 (d, J=2.6 Hz, 9H).

### Preparation 113B: tert-butyl (3-(((7R,14R)-1-(difluoromethoxy)-6-(methyl-d3)-5-oxo-5,6,7,14-tetrahydro-7,14 methanobenzo[f]benzo[4,5]imidazo[1,2-a][1,4]diazocin-11-yl)ethynyl)cyclobutyl)carbamate

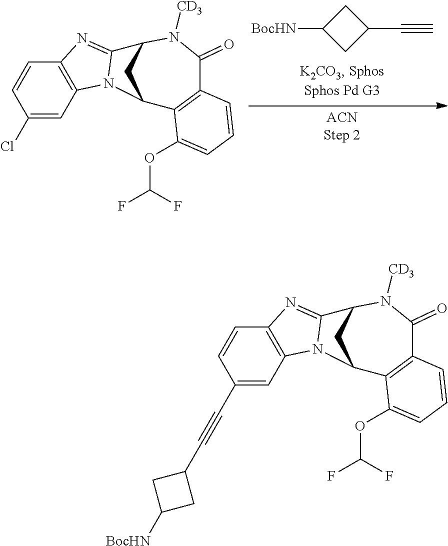 [SMILES: [2H]C([2H])([2H])N1C(=O)c2cccc(OC(F)F)c2[C@H]2C[C@@H]1c1nc3ccc(Cl)cc3n12 ; C#CC1CC(NC(=O)OC(C)(C)C)C1 ; [2H]C([2H])([2H])N1C(=O)c2cccc(OC(F)F)c2[C@H]2C[C@@H]1c1nc3ccc(C#CC4CC(NC(=O)OC(C)(C)C)C4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and tert-butyl N-(3-ethynylcyclobutyl)carbamate (79 mg, 0.408 mmol) in ACN (1 mL) were added SPhos Pd Gen.3 (32 mg, 0.041 mmol), SPhos (17 mg, 0.041 mmol) and K₂CO₃ (141 mg, 1.020 mmol) at room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 35% to 55% gradient in 20 min. This resulted in tert-butyl (3-(((7R,14R)-1-(difluoromethoxy)-6-(methyl-d₃)-5-oxo-5,6,7,14-tetrahydro-7,14 methanobenzo[f]benzo[4,5]imidazo[1,2-a][1,4]diazocin-11-yl)ethynyl)cyclobutyl)carbamate (66 mg, 58%) as a white solid. MS ESI calculated for C₃₀H₂₇D₃F₂N₄O₄[M+H]⁺, 552.24. found 552.30.

### Example 113: (7R,14R)-11-((3-aminocyclobutyl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

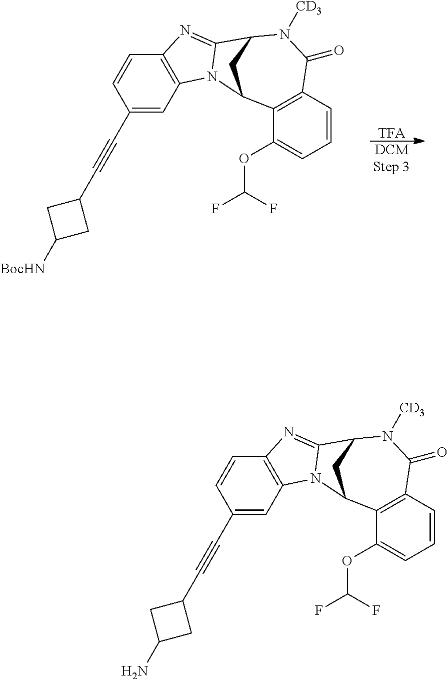 [SMILES: [2H]C([2H])([2H])N1C(=O)c2cccc(OC(F)F)c2[C@H]2C[C@@H]1c1nc3ccc(C#CC4CC(NC(=O)OC(C)(C)C)C4)cc3n12 ; "DCMStep 3" ; [2H]C([2H])([2H])N1C(=O)c2cccc(OC(F)F)c2[C@H]2C[C@@H]1c1nc3ccc(C#CC4CC(N)C4)cc3n12 ; "TFA"]

To a stirred solution of tert-butyl (3-(((7R,14R)-1-(difluoromethoxy)-6-(methyl-d₃)-5-oxo-5,6,7,14-tetrahydro-7,14 methanobenzo[f]benzo[4,5]imidazo[1,2-a][1,4]diazocin-11-yl)ethynyl)cyclobutyl)carbamate (66 mg, 0.120 mmol) in DCM (2 mL) was added TFA (0.5 mL) dropwise at room temperature. The resulting mixture was concentrated under reduced pressure. The residue was dissolved in MeOH (2 mL). The mixture was basified to pH 9 with TEA. The residue was purified by reversed-phase flash chromatography with the following conditions: column, C18 silica gel; mobile phase, CH₃CN in water (10 mmol/L NH₄HCO₃), 35% to 55% gradient in 20 min. This resulted in (7R,14R)-11-((3-aminocyclobutyl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (29 mg, 52%) as a white solid. MS ESI calculated for C₂₅H₁₉D₃F₂N₄O₂ [M+H]⁺ 452.19. found 452.05. ¹H NMR (400 MHz, Chloroform-d) δ 8.47 (d, J=8.2 Hz, 1H), 7.60 (d, J=8.0 Hz, 1H), 7.47 (s, 1H), 7.45-7.37 (m, 1H), 7.37-7.28 (m, 2H), 6.85 (t, J=72.7 Hz, 1H), 6.18 (d, J=7.3 Hz, 1H), 4.92 (d, J=7.1 Hz, 1H), 4.27-3.13 (m, 3H), 3.00-2.10 (m, 5H). ¹⁹F NMR (377 MHz, Chloroform-d) δ −79.85, −80.28, −81.19, −81.6

### Example 114: (7R,14R)-1-(difluoromethoxy)-6-(methyl-d₃)-11-(5-morpholinopent-1-yn-1-yl)-6,7-dihydro-7,14-methanobenzo[f]benzo[4,5]imidazo[1,2-a][1,4]diazocin-5(14H)-one

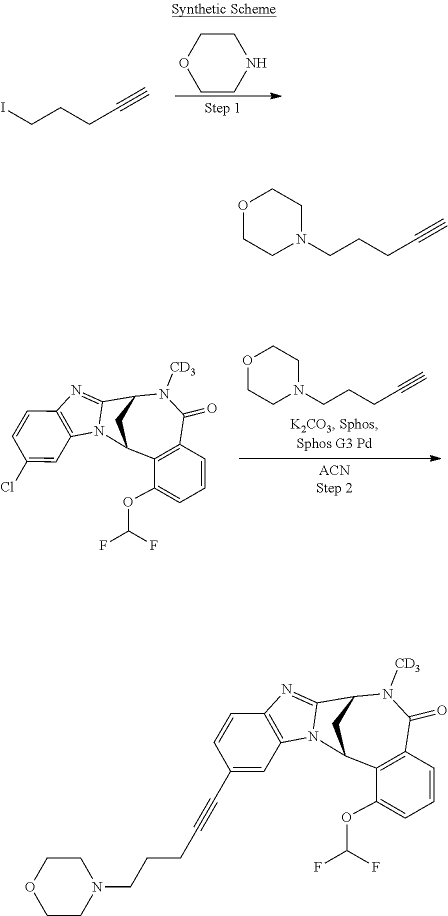

### Preparation 114A: 4-(pent-4-yn-1-yl)morpholine

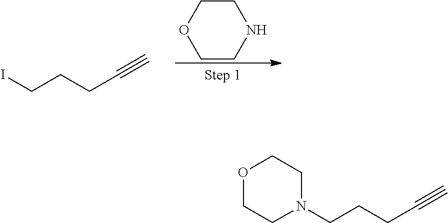

A mixture of 5-iodopent-1-yne (400 mg, 2.062 mmol) and morpholine (449 mg, 5.155 mmol) was stirred for 15 min at 80° C. The resulting mixture was diluted with diethyl ether (20 mL). The resulting mixture was filtered, the filter cake was washed with diethyl ether (2×5 mL). The organic layer was washed with 3×20 mL of HCl (2 N). Then, the aqueous layer was basified to pH 9 with NaOH (2 N). The resulting mixture was extracted with EtOAc (3×50 mL). The combined organic layers were washed with brine (1×50 mL), dried over anhydrous Na₂SO₄. After filtration, the filtrate was concentrated under reduced pressure. This resulted in 4-(pent-4-yn-1-yl)morpholine (306 mg, 96%) as a yellow oil. MS ESI calculated for C₉H₁₅NO [M+H]⁺, found N/A. ¹H NMR (400 MHz, Chloroform-d) δ 3.81-3.60 (m, 4H), 2.57-2.32 (m, 6H), 2.31-2.19 (m, 2H), 1.98-1.92 (m, 1H), 1.78-1.64 (m, 2H).

### Example 114: (7R,14R)-1-(difluoromethoxy)-6-(methyl-d₃)-11-(5-morpholinopent-1-yn-1-yl)-6,7-dihydro-7,14-methanobenzo[f]benzo[4,5]imidazo[1,2-a][1,4]diazocin-5(14H)-one

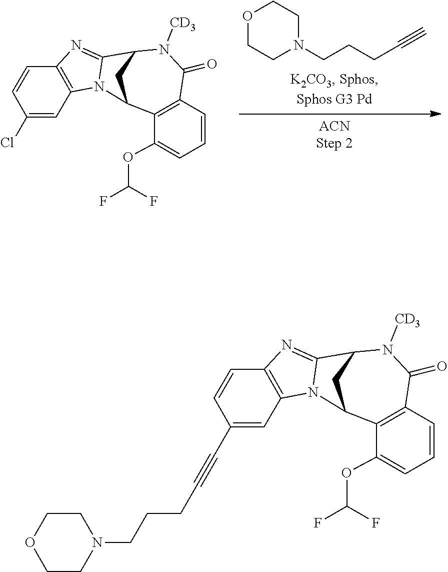

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and 4-(pent-4-yn-1-yl)morpholine (117 mg, 0.765 mmol) in ACN (2 mL) were added SPhos Pd Gen.3 (24 mg, 0.031 mmol), SPhos (13 mg, 0.031 mmol) and K₂CO₃ (106 mg, 0.765 mmol) at room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 35% to 55% gradient in 20 min. This resulted in (7R,14R)-1-(difluoromethoxy)-6-(methyl-d3)-11-(5-morpholinopent-1-yn-1-yl)-6,7-dihydro-7,14-methanobenzo[f]benzo[4,5]imidazo[1,2-a][1,4]diazocin-5(14H)-one (8 mg, 11%) as a white solid. MS ESI calculated for C₂₈H₂₅D₃F₂N₄O₃ [M+H]⁺, 510.23. found 510.10. ¹H NMR (400 MHz, Chloroform-d) δ 8.51-8.44 (m, 1H), 7.60 (d, J=8.4 Hz, 1H), 7.51-7.46 (m, 1H), 7.45-7.37 (m, 1H), 7.36-7.30 (m, 1H), 7.29-7.22 (m, 1H), 6.84 (t, J=72.8 Hz, 1H), 6.19 (d, J=7.2 Hz, 1H), 4.92 (d, J=7.1 Hz, 1H), 3.78-3.71 (m, 4H), 3.48-3.37 (m, 1H), 2.84 (d, J=13.5 Hz, 1H), 2.58-2.45 (m, 8H), 1.88-1.76 (m, 2H). ¹⁹F NMR (377 MHz, Chloroform-d) δ −79.88, −80.32, −80.87, −81.31.

### Example 115: (7R,14R)-1-(difluoromethoxy)-6-(methyl-d₃)-11-(3-((1-methylpiperidin-4-yl)oxy)prop-1-yn-1-yl)-6,7-dihydro-7,14-methanobenzo[f]benzo[4,5]imidazo[1,2-a][1,4]diazocin-5(14H)-one

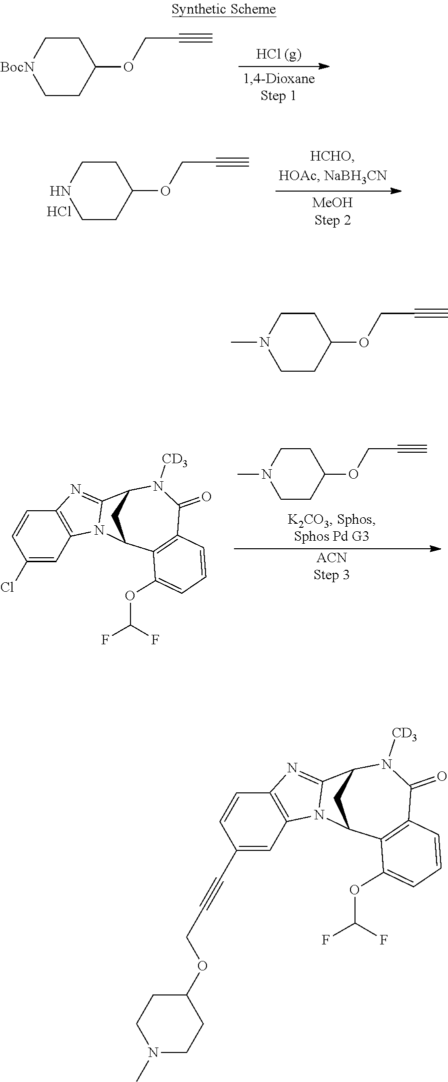

### Preparation 115A: 4-(prop-2-yn-1-yloxy)piperidine Hydrochloride

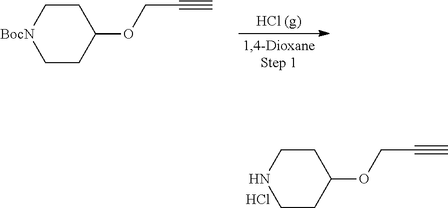

To a stirred solution of tert-butyl 4-(prop-2-yn-1-yloxy)piperidine-1-carboxylate (200 mg, 0.836 mmol) in 1,4-dioxane (2 mL) was added HCl in 1,4-dioxane (1 mL, 4M) dropwise at 0° C. The resulting mixture was stirred for 2 h at room temperature. The resulting mixture was concentrated under vacuum. The crude product was used in the next step directly without further purification. This resulted in 4-(prop-2-yn-1-yloxy)piperidine hydrochloride (140 mg, 95%) as a yellow solid. MS ESI calculated for C₈H₁₃NO [M+H]⁺, 140.10. found 140.30. ¹H NMR (400 MHz, Chloroform-d) δ 4.18 (d, J=2.4 Hz, 2H), 3.96-3.86 (m, 1H), 3.48-3.04 (m, 4H), 2.51-2.41 (m, 1H), 2.24-1.97 (m, 4H).

### Preparation 115B: 1-methyl-4-(prop-2-yn-1-yloxy)piperidine

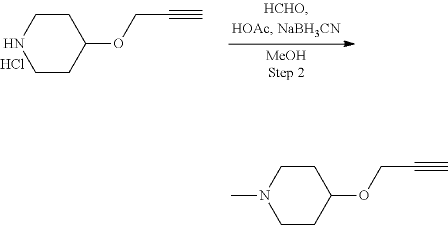

To a stirred solution of 4-(prop-2-yn-1-yloxy)piperidine hydrochloride (150 mg, 0.854 mmol), HCHO (139 mg, 1.708 mmol, 37%) and HOAc (128 mg, 2.135 mmol) in MeOH (2 mL) was added NaBH₃CN (80 mg, 1.281 mmol) in portions at 0° C. under nitrogen atmosphere. The resulting mixture was stirred for 16 h at room temperature under nitrogen atmosphere. The residue was basified to pH 9 with NaOH (2 N). The resulting mixture was extracted with CH₂Cl₂ (3×20 mL). The combined organic layers dried over anhydrous Na₂SO₄. After filtration, the filtrate was concentrated under reduced pressure. This resulted in 1-methyl-4-(prop-2-yn-1-yloxy)piperidine (120 mg, 91%) as a yellow oil. MS ESI calculated for C₉H₁₅NO [M+H]⁺, 154.12. found 154.35. ¹H NMR (400 MHz, Chloroform-d) δ 4.21-4.15 (m, 2H), 3.61-3.52 (m, 1H), 2.80-2.65 (m, 2H), 2.43-2.39 (m, 1H), 2.28 (s, 3H), 2.21-2.11 (m, 2H), 1.97-1.87 (m, 2H), 1.73-1.61 (m, 2H).

### Example 115: (7R,14R)-1-(difluoromethoxy)-6-(methyl-d₃)-11-(3-((1-methylpiperidin-4-yl)oxy)prop-1-yn-1-yl)-6,7-dihydro-7,14-methanobenzo[f]benzo[4,5]imidazo[1,2-a][1,4]diazocin-5(14H)-one

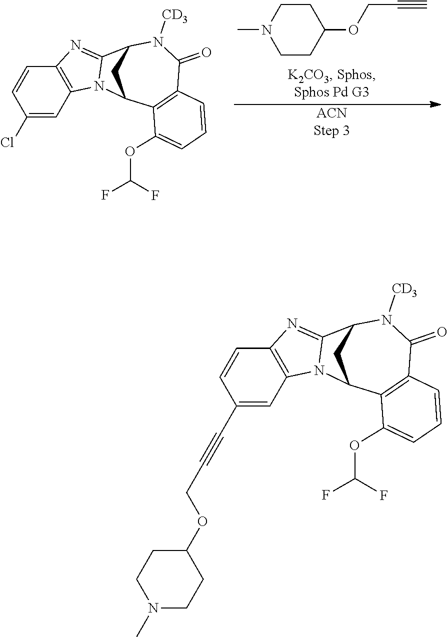

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and 1-methyl-4-(prop-2-yn-1-yloxy)piperidine (125 mg, 0.816 mmol) in CH₃CN (2 mL) were added SPhos Pd Gen.3 (32 mg, 0.041 mmol), SPhos (17 mg, 0.041 mmol) and K₂CO₃ (141 mg, 1.020 mmol) in portions at room temperature. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 35% to 55% gradient in 20 min. This resulted in (7R,14R)-1-(difluoromethoxy)-6-(methyl-d₃)-11-(3-((1-methylpiperidin-4-yl)oxy)prop-1-yn-1-yl)-6,7-dihydro-7,14-methanobenzo[f]benzo[4,5]imidazo[1,2-a][1,4]diazocin-5(14H)-one (17 mg, 15%) as a white solid. MS ESI calculated for C₂₈H₂₅D₃F₂N₄O₃ [M+H]⁺, 510.23. found 510.20. ¹H NMR (400 MHz, Chloroform-d) δ 8.53-8.44 (m, 1H), 7.66-7.60 (m, 1H), 7.55 (d, J=1.5 Hz, 1H), 7.42 (t, J=8.2 Hz, 1H), 7.36-7.28 (m, 2H), 7.03-6.64 (m, 1H), 6.21 (d, J=7.2 Hz, 1H), 4.93 (d, J=7.1 Hz, 1H), 4.43 (s, 2H), 3.73-3.60 (m, 1H), 3.48-3.36 (m, 1H), 2.86 (d, J=13.5 Hz, 1H), 2.74 (d, J=5.3 Hz, 2H), 2.30 (s, 3H), 2.26-2.13 (m, 2H), 2.03-1.94 (m, 2H), 1.78-7.68 (s, 2H). ¹⁹F NMR (377 MHz, Chloroform-d) δ −79.93, −80.38, −80.92, −81.36.

### Example 116: tert-butyl 4-((3-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)prop-2-yn-1-yl)oxy)piperidine-1-carboxylate

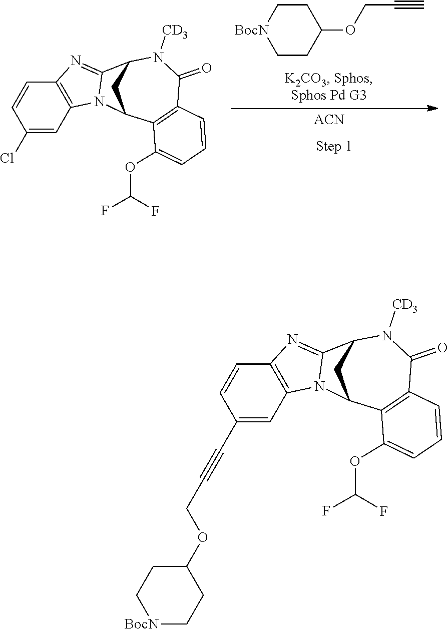 [SMILES: [2H]C([2H])([2H])N1C(=O)c2cccc(OC(F)F)c2[C@H]2C[C@@H]1c1nc3ccc(Cl)cc3n12 ; C#CCOC1CCN(C(=O)OC(C)(C)C)CC1 ; "ACN" ; [2H]C([2H])([2H])N1C(=O)c2cccc(OC(F)F)c2[C@H]2C[C@@H]1c1nc3ccc(C#CCOC4CCN(C(=O)OC(C)(C)C)CC4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and tert-butyl 4-(prop-2-yn-1-yloxy)piperidine-1-carboxylate (122 mg, 0.510 mmol) in ACN (3 mL) were added SPhos (21 mg, 0.051 mmol), SPhos Pd Gen.3 (40 mg, 0.051 mmol) and K₂CO₃ (176 mg, 1.275 mmol) room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 35% to 55% gradient in 20 min. This resulted in tert-butyl 4-((3-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)prop-2-yn-1-yl)oxy)piperidine-1-carboxylate (5 mg, 3%) as a white solid. MS ESI calculated for C₃₂H₃₁D₃F₂N₄O₅ [M+H]⁺, 596.27. found 596.30. ¹H NMR (400 MHz, Chloroform-d) δ 8.49 (d, J=8.1 Hz, 1H), 7.67-7.62 (m, 1H), 7.56 (s, 1H), 7.47-7.39 (m, 1H), 7.38-7.30 (m, 2H), 6.85 (t, J=72.8 Hz, 1H), 6.23 (d, J=7.2 Hz, 1H), 5.04-4.96 (m, 1H), 4.44 (s, 2H), 3.89-3.69 (m, 3H), 3.54-3.40 (m, 1H), 3.20-3.07 (m, 2H), 2.87 (d, J=13.5 Hz, 1H), 1.95-1.86 (m, 2H), 1.65-1.53 (m, 2H), 1.46 (s, 9H). ¹⁹F NMR (377 MHz, Chloroform-d) δ −80.08, −80.53, −81.02, −81.46.

### Example 117: (7R,14R)-1-(difluoromethoxy)-6-(methyl-d₃)-11-(3-(piperidin-4-yloxy)prop-1-yn-1-yl)-6,7-dihydro-7,14-methanobenzo[f]benzo[4,5]imidazo[1,2-a][1,4]diazocin-5(14H)-one

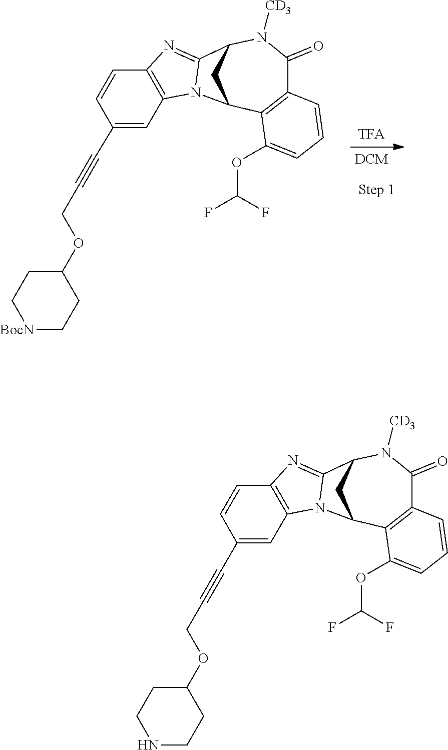 [SMILES: [2H]C([2H])([2H])N1C(=O)c2cccc(OC(F)F)c2[C@H]2C[C@@H]1c1nc3ccc(C#CCOC4CCN(C(=O)OC(C)(C)C)CC4)cc3n12 ; [2H]C([2H])([2H])N1C(=O)c2cccc(OC(F)F)c2[C@H]2C[C@@H]1c1nc3ccc(C#CCOC4CCNCC4)cc3n12]

To a stirred solution of tert-butyl 4-((3-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)prop-2-yn-1-yl)oxy)piperidine-1-carboxylate (20 mg, 0.034 mmol) in DCM (1.5 mL) was added TFA (0.3 mL) dropwise at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 35% to 55% gradient in 20 min. This resulted in (7R,14R)-1-(difluoromethoxy)-6-(methyl-d₃)-11-(3-(piperidin-4-yloxy)prop-1-yn-1-yl)-6,7-dihydro-7,14-methanobenzo[f]benzo[4,5]imidazo[1,2-a][1,4]diazocin-5(14H)-one (8 mg, 48%) as a white solid. MS ESI calculated for C₂₇H₂₃D₃F₂N₄O₃ [M+H]⁺, 496.22. found 496.15. ¹H NMR (400 MHz, Chloroform-d) δ 8.50-8.43 (m, 1H), 7.63 (d, J=8.4 Hz, 1H), 7.56-7.52 (m, 1H), 7.42 (t, J=8.2 Hz, 1H), 7.37-7.28 (m, 2H), 6.85 (t, J=72.8 Hz, 1H), 6.21 (d, J=7.3 Hz, 1H), 4.94 (d, J=7.1 Hz, 1H), 4.44 (s, 2H), 3.77-3.67 (m, 1H), 3.49-3.36 (m, 1H), 3.19-3.06 (m, 2H), 2.85 (d, J=13.5 Hz, 1H), 2.75-2.64 (m, 2H), 2.08-1.97 (m, 2H), 1.62-1.49 (m, 2H). ¹⁹F NMR (377 MHz, Chloroform-d) δ −80.00, −80.77, −80.97, −81.41.

### Example 118: (7R,14R)-1-(difluoromethoxy)-11-((1-ethylazetidin-3-yl)ethynyl)-6-(methyl-d₃)-6,7-dihydro-7,14-methanobenzo[f]benzo[4,5]imidazo[1,2-a][1,4]diazocin-5(14H)-one

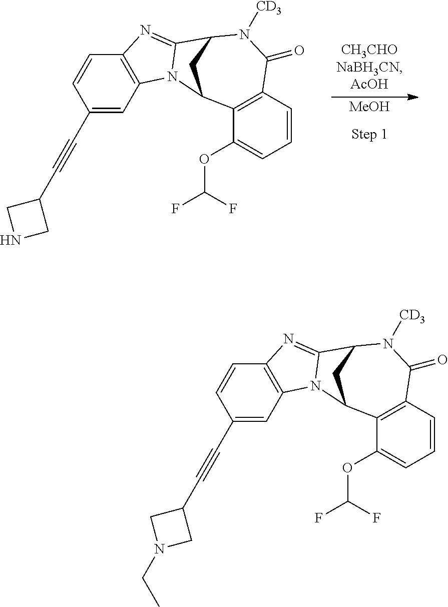 [SMILES: "MeOH" ; [2H]C([2H])([2H])N1C(=O)c2cccc(OC(F)F)c2[C@H]2C[C@@H]1c1nc3ccc(C#CC4CNC4)cc3n12 ; [2H]C([2H])([2H])N1C(=O)c2cccc(OC(F)F)c2[C@H]2C[C@@H]1c1nc3ccc(C#CC4CN(CC)C4)cc3n12]

To a solution of (7R,14R)-11-(azetidin-3-ylethynyl)-1-(difluoromethoxy)-6-(methyl-d₃)-6,7-dihydro-7,14-methanobenzo[f]benzo[4,5]imidazo[1,2-a][1,4]diazocin-5(14H)-one (35 mg, 0.080 mmol), AcOH (14 mg, 0.240 mmol) and acetaldehyde (111 mg, 0.240 mmol) in MeOH (0.5 mL) was added NaBH₃CN (8 mg, 0.120 mmol). The solution was stirred for 1 h at room temperature. The resulting mixture was concentrated under reduced pressure. The residue was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min; Gradient: 31% B to 48% B in 10 min; Wave Length: 254/220 nm; RT1(min): 10.03) to afford (7R,14R)-1-(difluoromethoxy)-11-((1-ethylazetidin-3-yl)ethynyl)-6-(methyl-d₃)-6,7-dihydro-7,14-methanobenzo[f]benzo[4,5]imidazo[1,2-a][1,4]diazocin-5(14H)-one (3 mg, 9%) as a white solid. MS ESI calculated for C₂₆H₂₁D₃F₂N₄O₂ [M+H]⁺, 466.21. found 466.10. ¹H NMR (400 MHz, DMSO-d₆) δ 8.27 (d, J=4.4 Hz, 1H), 7.78-7.56 (m, 2H), 7.53-7.36 (m, 3H), 7.20 (d, J=8.5 Hz, 1H), 6.21 (d, J=7.1 Hz, 1H), 5.21 (d, J=7.1 Hz, 1H), 3.58-3.44 (m, 3H), 3.40-3.36 (m, 1H), 2.94 (t, J=6.8 Hz, 2H), 2.80 (d, J=13.8 Hz, 1H), 2.41-2.32 (m, 2H), 0.86 (t, J=7.2 Hz, 3H). ¹⁹F NMR (377 MHz, DMSO-d₆) δ −81.64, −82.09, −82.14, −82.59.

### Example 119: (7R,14R)-1-(difluoromethoxy)-11-((1-isopropylazetidin-3-yl)ethynyl)-6-(methyl-d3)-6,7-dihydro-7,14-methanobenzo[f]benzo[4,5]imidazo[1,2-a][1,4]diazocin-5(14H)-one

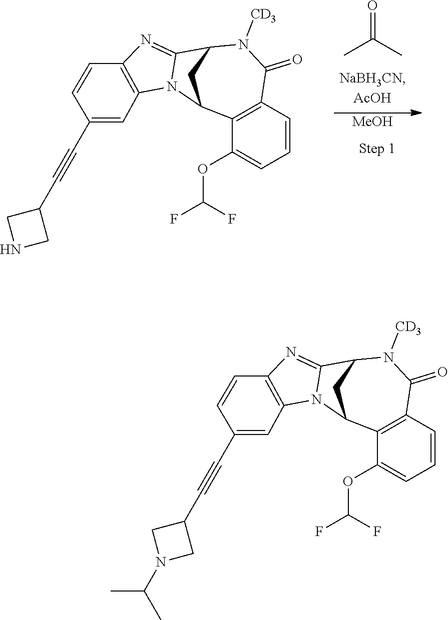

To a solution of (7R,14R)-11-(azetidin-3-ylethynyl)-1-(difluoromethoxy)-6-(methyl-d₃)-6,7-dihydro-7,14-methanobenzo[f]benzo[4,5]imidazo[1,2-a][1,4]diazocin-5(14H)-one (35 mg, 0.080 mmol), AcOH (14 mg, 0.240 mmol) and acetone (7 mg, 0.120 mmol) in MeOH (0.5 mL) was added NaBH₃CN (8 mg, 0.120 mmol). The solution was stirred for 1 h at room temperature. The resulting mixture was concentrated under reduced pressure. The residue was purified by Prep-HPLC with the following conditions (Column: XBridge Prep OBD C18 Column 30*150 mm, 5m; Mobile Phase A: Water (10 mmol/L NH₄HCO₃+0.05% NH₃·H₂O), Mobile Phase B: ACN; Flow rate: 60 mL/min; Gradient: 28% B to 48% B in 10 min; Wave Length: 254/220 nm; RT1(min): 10.28) to afford (7R,14R)-1-(difluoromethoxy)-11-((1-isopropylazetidin-3-yl)ethynyl)-6-(methyl-d3)-6,7-dihydro-7,14-methanobenzo[f]benzo[4,5]imidazo[1,2-a][1,4]diazocin-5(14H)-one (10 mg, 27%) as a white solid. MS ESI calculated for C₂₇H₂₃D₃F₂N₄O₂ [M+H]⁺, 480.22. found 480.15. ¹H NMR (400 MHz, Chloroform-d) δ 8.47 (d, J=8.2 Hz, 1H), 7.64-7.58 (m, 1H), 7.53-7.48 (m, 1H), 7.41 (t, J=8.2 Hz, 1H), 7.34 (d, J=8.2 Hz, 1H), 6.85 (t, J=72.7 Hz, 1H), 6.20 (d, J=7.1 Hz, 1H), 4.95-4.89 (m, 1H), 3.79-3.68 (m, 2H), 3.54-3.36 (m, 2H), 3.17 (s, 2H), 2.84 (d, J=13.5 Hz, 1H), 2.48-2.26 (m, 2H), 0.98 (d, J=6.2 Hz, 6H). ¹⁹F NMR (377 MHz, Chloroform-d) δ −79.91, −80.35, −81.13, −81.58.

### Example 120: (7R,14R)-11-((1-acetylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

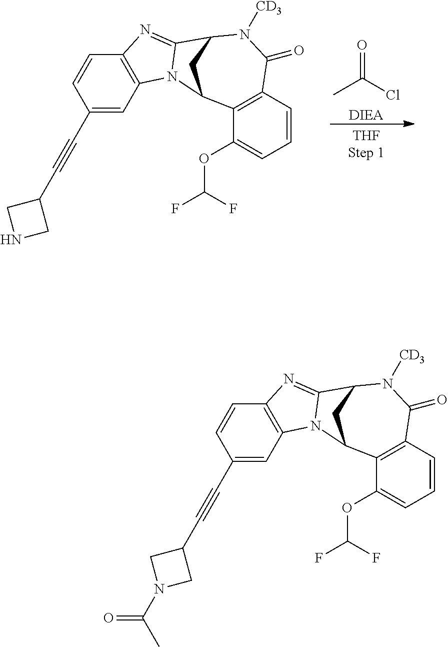

To a solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (35 mg, 0.080 mmol) and DIEA (21 mg, 0.160 mmol) in THF (0.5 mL) was added acetyl chloride (9 mg, 0.120 mmol) at 0° C. under nitrogen atmosphere. The solution was stirred for 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 35% to 55% gradient in 20 min. This resulted in (7R,14R)-11-((1-acetylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (13 mg, 35%) as a white solid. MS ESI calculated for C₂₆H₁₉D₃F₂N₄O₃ [M+H]⁺, 480.18. found 480.10. ¹H NMR (400 MHz, DMSO-d₆) δ 8.33-8.23 (m, 1H), 7.79-7.58 (m, 2H), 7.54-7.38 (m, 3H), 7.25 (d, J=8.4 Hz, 1H), 6.23 (d, J=7.0 Hz, 1H), 5.22 (d, J=7.1 Hz, 1H), 4.43 (t, J=8.4 Hz, 1H), 4.25-4.05 (m, 2H), 3.87-3.77 (m, 1H), 3.75-3.67 (m, 1H), 3.57-3.44 (m, 1H), 2.81 (d, J=13.8 Hz, 1H), 1.77 (s, 3H). ¹⁹F NMR (377 MHz, DMSO-d₆) δ −82.02, −82.05, −82.11.

### Example 121: (7R,14R)-1-(difluoromethoxy)-6-(methyl-d3)-11-((1-(methylsulfonyl)azetidin-3-yl)ethynyl)-6,7-dihydro-7,14-methanobenzo[f]benzo[4,5]imidazo[1,2-a][1,4]diazocin-5(14H)-one

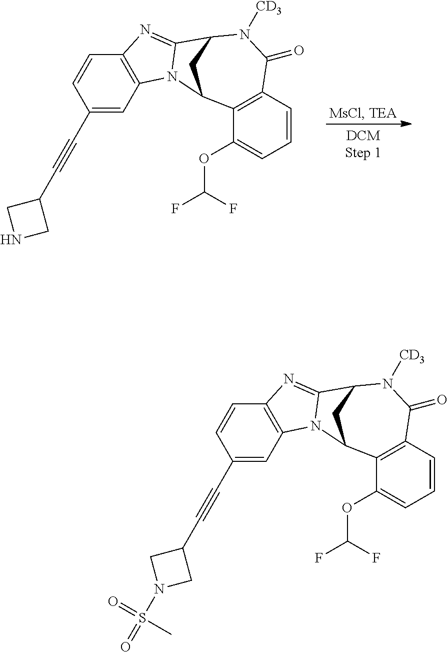 [SMILES: [2H]C([2H])([2H])N1C(=O)c2cccc(OC(F)F)c2[C@H]2C[C@@H]1c1nc3ccc(C#CC4CNC4)cc3n12 ; "MsCl, TEA" ; [2H]C([2H])([2H])N1C(=O)c2cccc(OC(F)F)c2[C@H]2C[C@@H]1c1nc3ccc(C#CC4CN(S(C)(=O)=O)C4)cc3n12]

To a solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (35 mg, 0.080 mmol) and TEA (24 mg, 0.240 mmol) in DCM (0.5 mL) was added methanesulfonyl chloride (14 mg, 0.120 mmol) at 0° C. under nitrogen atmosphere. The solution was stirred for 1 h at room temperature. The resulting mixture was concentrated under reduced pressure. The residue was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: CH₃CN; Flow rate: 60 mL/min; Gradient: 20% B to 37% B in 10 min; Wave Length: 254/220 nm; RT1(min): 15.18) to afford (7R,14R)-1-(difluoromethoxy)-6-(methyl-d3)-11-((1-(methylsulfonyl)azetidin-3-yl)ethynyl)-6,7-dihydro-7,14-methanobenzo[f]benzo[4,5]imidazo[1,2-a][1,4]diazocin-5(14H)-one (7 mg, 16%) as a white solid. MS ESI calculated for C₂₅H₁₉D₃F₂N₄O₄S [M+H]⁺, 516.15. found 516.05. ¹H NMR (400 MHz, Chloroform-d) δ 8.53-8.45 (m, 1H), 7.64 (d, J=8.4 Hz, 1H), 7.52 (s, 1H), 7.47-7.40 (m, 1H), 7.39-7.31 (m, 1H), 7.30-7.27 (m, 1H), 6.85 (t, J=72.7 Hz, 1H), 6.22 (d, J=7.1 Hz, 1H), 4.96 (d, J=6.9 Hz, 1H), 4.24-4.15 (m, 2H), 4.10-4.03 (m, 2H), 3.70-3.59 (m, 1H), 3.50-3.38 (m, 1H), 2.92 (s, 3H), 2.86 (d, J=13.5 Hz, 1H). ¹⁹F NMR (377 MHz, Chloroform-d) δ −80.07, −80.51, −80.94, −81.39.

### Example 122: (7R,14R)-1-(difluoromethoxy)-11-(4-ethoxybut-1-yn-1-yl)-6-(methyl-d3)-6,7-dihydro-7,14-methanobenzo[f]benzo[4,5]imidazo[1,2-a][1,4]diazocin-5(14H)-one

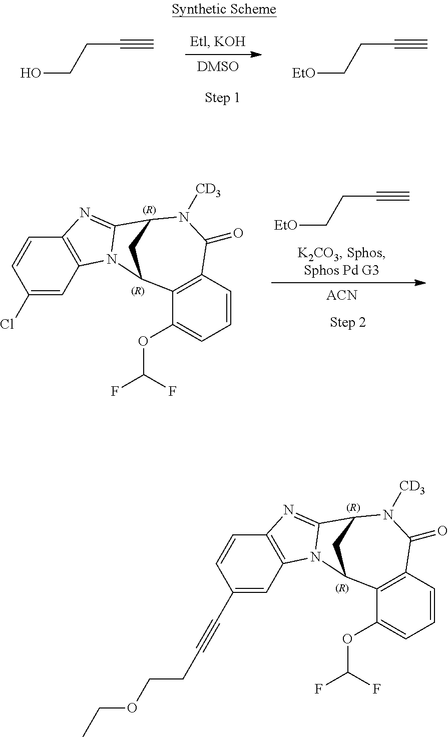

### Preparation 122A: 4-ethoxybut-1-yne

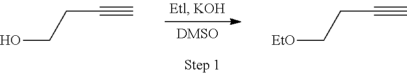

To a stirred solution of KOH (4.45 g, 79.268 mmol) and 3-butyn-1-ol (5 mL, 66.057 mmol) in DMSO (12.5 mL) was added ethyl iodide (5.8 mL, 72.641 mmol) dropwise at 0° C. under nitrogen atmosphere. The resulting mixture was stirred for 2 h at 40° C. under nitrogen atmosphere. The upper layer was washed with water (3×6 mL) then washed with brine (3×6 mL), dried over anhydrous Na₂SO₄. After filtration to afford crude product 4-ethoxybut-1-yne (6.01 g, 93%) as a yellow oil. MS ESI calculated for C₆H₁₀O [M+H]⁺, 99.07. found N/A. ¹H NMR (300 MHz, Chloroform-d) δ 3.62-3.49 (m, 4H), 2.52-2.43 (m, 2H), 2.00 (t, J=2.7 Hz, 1H), 1.23 (t, J=7.0 Hz, 3H).

### Example 122: (7R,14R)-1-(difluoromethoxy)-11-(4-ethoxybut-1-yn-1-yl)-6-(methyl-d3)-6,7-dihydro-7,14-methanobenzo[f]benzo[4,5]imidazo[1,2-a][1,4]diazocin-5(14H)-one

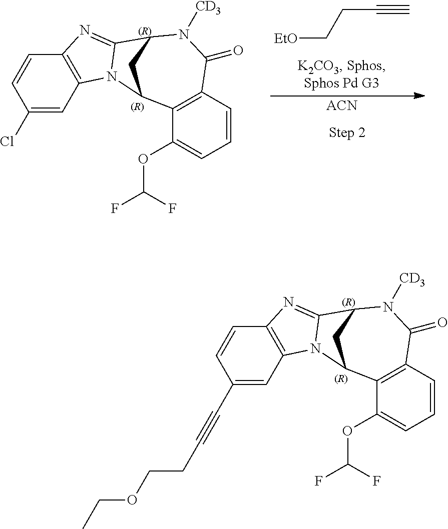

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol) and K₂CO₃ (106 mg, 0.765 mmol) in ACN (1 mL) were added SPhos (13 mg, 0.031 mmol), SPhos Pd Gen.3 (24 mg, 0.031 mmol) and 4-ethoxybut-1-yne (75 mg, 0.764 mmol) at room temperature under nitrogen atmosphere. The resulting mixture was stirred for 16 h at 80° C. under nitrogen atmosphere. The residue was purified by silica gel column chromatography, eluted with CH₂Cl₂/MeOH (5/1) followed by reversed-phase flash chromatography with the following conditions: column, C18 silica gel; mobile phase, CH₃CN in Water (10 mmol/L NH₄HCO₃), 40% to 60% gradient in 20 min to afford (7R,14R)-1-(difluoromethoxy)-11-(4-ethoxybut-1-yn-1-yl)-6-(methyl-d₃)-6,7-dihydro-7,14-methanobenzo[f]benzo[4,5]imidazo[1,2-a][1,4]diazocin-5(14H)-one (32 mg, 46%) as an off-white solid. MS ESI calculated for C₂₅H₂₀D₃F₂N₃O₃[M+H]⁺, 455.19. found 455.15. ¹H NMR (400 MHz, DMSO-d₆) δ 8.31-8.25 (m, 1H), 7.58 (d, J=6.3 Hz, 1H), 7.56 (t, J=72 Hz, 1H), 7.51 (d, J=6.9 Hz, 2H), 7.44-7.41 (m, 1H), 7.21-7.16 (m, 1H), 6.21 (d, J=7.1 Hz, 1H), 5.21 (d, J=7.1 Hz, 1H), 3.60-3.43 (m, 5H), 2.80 (d, J=13.8 Hz, 1H), 2.65 (t, J=6.7 Hz, 2H), 1.13 (t, J=7.0 Hz, 3H). ¹⁹F NMR (377 MHz, DMSO-d₆) δ −81.99.

### Example 123: (7R,14R)-11-(4-aminobut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

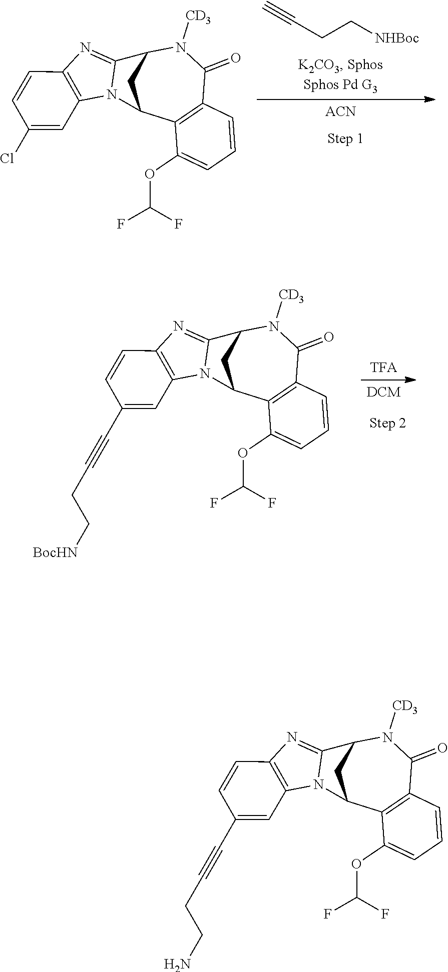 [SMILES: [2H]C([2H])([2H])N1C(=O)c2cccc(OC(F)F)c2[C@H]2C[C@@H]1c1nc3ccc(Cl)cc3n12 ; C#CCCNC(=O)OC(C)(C)C ; "ACN" ; [2H]C([2H])([2H])N1C(=O)c2cccc(OC(F)F)c2[C@H]2C[C@@H]1c1nc3ccc(C#CCCNC(=O)OC(C)(C)C)cc3n12 ; "TFA" ; [2H]C([2H])([2H])N1C(=O)c2cccc(OC(F)F)c2[C@H]2C[C@@H]1c1nc3ccc(C#CCCN)cc3n12]

### Preparation 123A: tert-butyl (4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl)carbamate

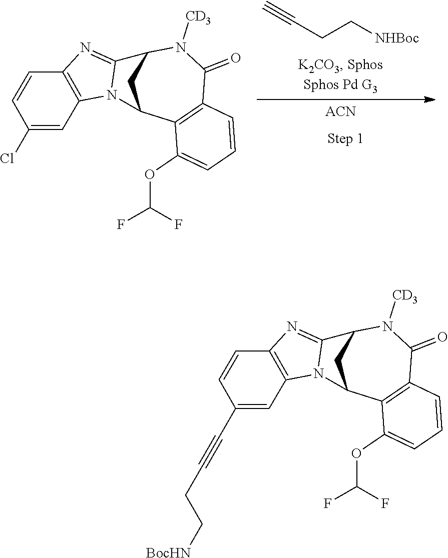 [SMILES: [2H]C([2H])([2H])N1C(=O)c2cccc(OC(F)F)c2[C@H]2C[C@@H]1c1nc3ccc(Cl)cc3n12 ; C#CCCNC(=O)OC(C)(C)C ; "ACN" ; [2H]C([2H])([2H])N1C(=O)c2cccc(OC(F)F)c2[C@H]2C[C@@H]1c1nc3ccc(C#CCCNC(=O)OC(C)(C)C)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and K₂CO₃ (176 mg, 1.275 mmol) in ACN (1 mL) were added SPhos (21 mg, 0.051 mmol), SPhos Pd Gen.3 (40 mg, 0.051 mmol) and tert-butyl but-3-yn-1-ylcarbamate (216 mg, 1.273 mmol) at room temperature under nitrogen atmosphere. The resulting mixture was stirred for 16 h at 80° C. under nitrogen atmosphere. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min to afford tert-butyl (4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl)carbamate (80 mg, 60%) as a white solid. MS ESI calculated for C₂₈H₂₅D₃F₂N₄O₄[M+H]⁺, 526.23. found 526.25. ¹H NMR (400 MHz, Chloroform-d) δ 8.51-8.45 (m, 1H), 7.63 (d, J=8.4 Hz, 1H), 7.52 (s, 1H), 7.43 (t, J=8.2 Hz, 1H), 7.38-7.28 (m, 2H), 6.96 (t, J=72.7 Hz, 1H), 6.23 (d, J=5.5 Hz, 1H), 4.95 (d, J=49.7 Hz, 2H), 3.54-3.31 (m, 3H), 2.87 (d, J=12.4 Hz, 1H), 2.62 (t, J=6.5 Hz, 2H), 1.45 (s, 9H).

### Example 123: (7R,14R)-11-(4-aminobut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

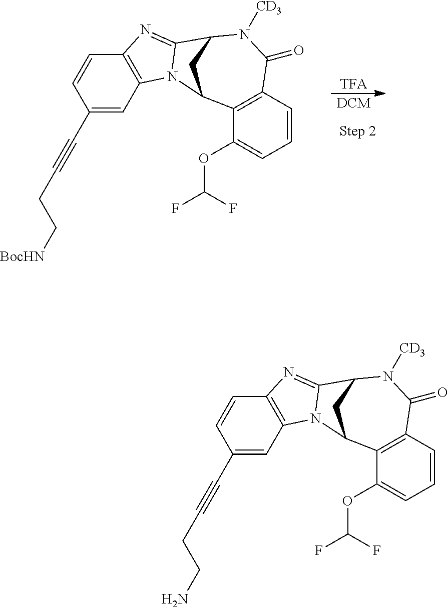

To a stirred solution of tert-butyl (4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl)carbamate (90 mg, 0.171 mmol) in DCM (3 mL) was added TFA (0.6 mL) at room temperature under nitrogen atmosphere. The resulting mixture was stirred for 2 h at room temperature under nitrogen atmosphere. The resulting mixture was concentrated under vacuum. The residue was purified by reversed-phase flash chromatography with the following conditions: column, C18 silica gel; mobile phase, CH₃CN in Water (10 mmol/L NH₄HCO₃), 30% to 50% gradient in 20 min to afford (7R,14R)-11-(4-aminobut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (37 mg, 50%) as a white solid. MS ESI calculated for C₂₃H₁₇D₃F₂N₄O₂ [M+H]⁺, 426.17. found 426.15. ¹H NMR (400 MHz, DMSO-d₆) δ 8.29-8.25 (m, 1H), 7.58 (d, J=3.8 Hz, 1H), 7.56 (t, J=74 Hz, 1H), 7.53-7.47 (m, 2H), 7.43 (d, J=1.5 Hz, 1H), 7.23-7.19 (m, 1H), 6.21 (d, J=7.2 Hz, 1H), 5.21 (d, J=7.1 Hz, 1H), 3.54-3.44 (m, 1H), 2.79 (d, J=13.8 Hz, 1H), 2.73 (t, J=6.8 Hz, 2H), 2.46 (d, J=6.8 Hz, 2H). ¹⁹F NMR (377 MHz, DMSO-d₆) δ −82.05, −82.09.

### Example 124: (7R,14R)-1-(difluoromethoxy)-6-(methyl-d₃)-11-((1-(2,2,2-trifluoroethyl)azetidin-3-yl)ethynyl)-6,7-dihydro-7,14-methanobenzo[f]benzo[4,5]imidazo[1,2-a][1,4]diazocin-5(14H)-one

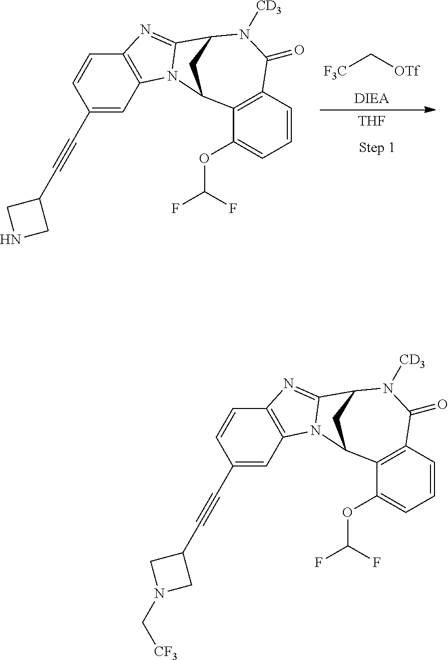

To a solution of (7R,14R)-11-(azetidin-3-ylethynyl)-1-(difluoromethoxy)-6-(methyl-d₃)-6,7-dihydro-7,14-methanobenzo[f]benzo[4,5]imidazo[1,2-a][1,4]diazocin-5(14H)-one (35 mg, 0.080 mmol) and DIEA (21 mg, 0.160 mmol) in THF (0.5 mL) was added 2,2,2-trifluoroethyl trifluoromethanesulfonate (28 mg, 0.120 mmol). The solution was stirred for 1 h at room temperature. The residue was purified by Prep-HPLC with the following conditions (Column: XBridge Shield RP18 OBD Column 30*150 mm, 5m; Mobile Phase A: Water (10 mmol/L NH₄HCO₃), Mobile Phase B: CH₃CN; Flow rate: 60 mL/min; Gradient: 32% B to 52% B in 10 min; Wave Length: 254/200 nm; RT1(min): 11.18) to afford (7R,14R)-1-(difluoromethoxy)-6-(methyl-d₃)-11-((1-(2,2,2-trifluoroethyl)azetidin-3-yl)ethynyl)-6,7-dihydro-7,14-methanobenzo[f]benzo[4,5]imidazo[1,2-a][1,4]diazocin-5(14H)-one (8 mg, 19%) as a white solid. MS ESI calculated for C₂₆H₁₈D₃F₅N₄O₂[M+H]⁺, 520.18. found 520.15. ¹H NMR (400 MHz, DMSO-d₆) δ 8.28 (d, J=6.9 Hz, 1H), 7.76-7.55 (m, 2H), 7.54-7.48 (m, 2H), 7.47-7.36 (m, 1H), 7.23 (d, J=8.4 Hz, 1H), 6.23 (d, J=7.0 Hz, 1H), 5.21 (d, J=7.0 Hz, 1H), 3.72 (t, J=7.1 Hz, 2H), 3.65-3.53 (m, 2H), 3.32 (t, J=6.9 Hz, 2H), 3.29-3.17 (m, 2H), 2.80 (d, J=13.8 Hz, 1H). ¹⁹F NMR (377 MHz, DMSO-d₆) δ −69.68, −82.03.

### Example 125: (7R,14R)-1-(difluoromethoxy)-6-(methyl-d₃)-11-((1-pivaloylazetidin-3-yl)ethynyl)-6,7-dihydro-7,14-methanobenzo[f]benzo[4,5]imidazo[1,2-a][1,4]diazocin-5(14H)-one

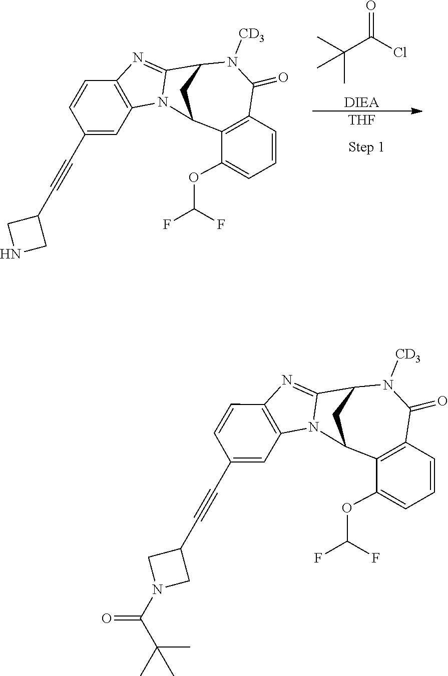

To a solution of (7R,14R)-11-(azetidin-3-ylethynyl)-1-(difluoromethoxy)-6-(methyl-d₃)-6,7-dihydro-7,14-methanobenzo[f]benzo[4,5]imidazo[1,2-a][1,4]diazocin-5(14H)-one (35 mg, 0.080 mmol) and DIEA (21 mg, 0.160 mmol) in THF (0.5 mL) was added 2,2-dimethylpropanoyl chloride (14 mg, 0.120 mmol) at 0° C. under nitrogen atmosphere. The solution was stirred for 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 35% to 55% gradient in 20 min. This resulted in (7R,14R)-1-(difluoromethoxy)-6-(methyl-d₃)-11-((1-pivaloylazetidin-3-yl)ethynyl)-6,7-dihydro-7,14-methanobenzo[f]benzo[4,5]imidazo[1,2-a][1,4]diazocin-5(14H)-one (14 mg, 32%) as a white solid. MS ESI calculated for C₂₉H₂₅D₃F₂N₄O₃[M+H]⁺, 522.23. found 522.20. ¹H NMR (400 MHz, DMSO-d₆) δ 8.27 (d, J=6.9, 2.6 Hz, 1H), 7.82-7.56 (m, 2H), 7.56-7.38 (m, 3H), 7.28-7.18 (m, 1H), 6.23 (d, J=7.1 Hz, 1H), 5.22 (d, J=7.1 Hz, 1H), 4.86-4.54 (m, 1H), 4.51-3.98 (m, 2H), 3.94-3.76 (m, 1H), 3.76-3.67 (m, 1H), 3.55-3.42 (m, 1H), 2.80 (d, J=13.8 Hz, 1H), 1.12 (s, 9H). ¹⁹F NMR (377 MHz, DMSO-d₆) δ −81.65, −82.10, −82.15, −82.60.

### Example 126: (7R,14R)-1-(difluoromethoxy)-11-((1-(2-hydroxy-2-methylpropanoyl)azetidin-3-yl)ethynyl)-6-(methyl-d₃)-6,7-dihydro-7,14-methanobenzo[f]benzo[4,5]imidazo[1,2-a][1,4]diazocin-5(14H)-one

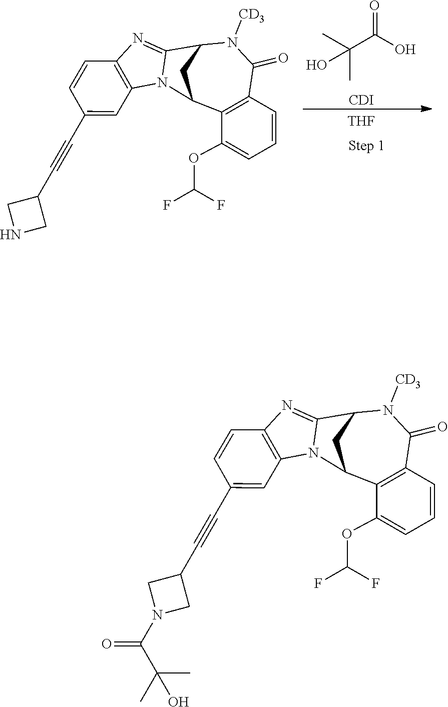

A mixture of acetonate (17 mg, 0.160 mmol) and CDI (26 mg, 0.160 mmol) in THF (0.5 mL) was stirred for 10 min at room temperature. To the above mixture was added (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (35 mg, 0.080 mmol). The resulting mixture was stirred for additional 1 h at room temperature. The resulting mixture was concentrated under reduced pressure. The residue was purified by Prep-HPLC with the following conditions (Column: XBridge Shield RP18 OBD Column 30*150 mm, 5m; Mobile Phase A: Water (10 mmol/L NH₄HCO₃), Mobile Phase B: MeOH; Flow rate: 60 mL/min; Gradient: 38% B to 57% B in 10 min; Wave Length: 254/200 nm; RT1(min): 10.93) to afford (7R,14R)-1-(difluoromethoxy)-11-((1-(2-hydroxy-2-methylpropanoyl)azetidin-3-yl)ethynyl)-6-(methyl-d₃)-6,7-dihydro-7,14-methanobenzo[f]benzo[4,5]imidazo[1,2-a][1,4]diazocin-5(14H)-one (10 mg, 24%) as a white solid. MS ESI calculated for C₂₈H₂₃D₃F₂N₄O₄ [M+H]⁺, 524.21. found 524.20. ¹H NMR (400 MHz, Chloroform-d) δ 8.53-8.46 (m, 1H), 7.69 (d, J=8.5 Hz, 1H), 7.59-7.54 (m, 1H), 7.46 (t, J=8.2 Hz, 1H), 7.38-7.34 (m, 2H), 6.85 (t, J=72.6 Hz, 1H), 6.29 (d, J=7.1 Hz, 1H), 5.16 (d, J=7.0 Hz, 1H), 4.69-4.65 (m, 1H), 4.51-4.31 (m, 2H), 4.19-4.14 (m, 1H), 3.76-3.60 (m, 1H), 3.59-3.46 (m, 1H), 2.92 (d, J=13.6 Hz, 1H), 1.44 (s, 6H). ¹⁹F NMR (377 MHz, Chloroform-d) δ −80.23, −80.68, −80.93, −81.38.

### Example 127: (7R,14R)-11-((1-(cyclopropanecarbonyl)azetidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

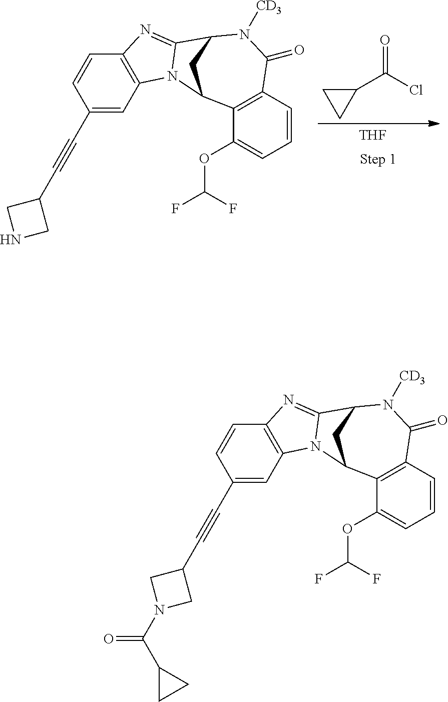

To a solution of (7R,14R)-11-(azetidin-3-ylethynyl)-1-(difluoromethoxy)-6-(methyl-d₃)-6,7-dihydro-7,14-methanobenzo[f]benzo[4,5]imidazo[1,2-a][1,4]diazocin-5(14H)-one (35 mg, 0.080 mmol) in THF (0.5 mL) was added 2,2-dimethylpropanoyl chloride (14 mg, 0.120 mmol) at 0° C. under nitrogen atmosphere. The solution was stirred for 1 h at room temperature. The resulting mixture was concentrated under reduced pressure. The residue was purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: CH₃CN; Flow rate: 60 mL/min; Gradient: 25% B to 40% B in 10 min; Wave Length: 254/220 nm; RT1(min): 11.889) to afford (7R,14R)-11-((1-(cyclopropanecarbonyl)azetidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (11 mg, 28%) as a white solid. MS ESI calculated for C₂₈H₂₁D₃F₂N₄O₃ [M+H]⁺, 506.20. found 506.15. ¹H NMR (400 MHz, Chloroform-d) δ 8.52-8.46 (m, 1H), 7.66 (d, J=8.5 Hz, 1H), 7.57-7.51 (m, 1H), 7.48-7.41 (m, 1H), 7.38-7.30 (m, 2H), 6.85 (t, J=72.7 Hz, 1H), 6.24 (d, J=7.2 Hz, 1H), 5.01 (d, J=7.1 Hz, 1H), 4.64-4.51 (m, 1H), 4.42-4.26 (m, 2H), 4.18-4.00 (m, 1H), 3.75-3.60 (m, 1H), 3.54-3.36 (m, 1H), 2.88 (d, J=13.6 Hz, 1H), 1.49-1.33 (m, 1H), 1.06-0.91 (m, 2H), 0.84-0.67 (m, 2H). ¹⁹F NMR (377 MHz, Chloroform-d) δ −80.02, −80.47, −81.02, −81.47.

### Example 128: (7R,14R)-1-(difluoromethoxy)-11-((R)-4-hydroxy-3-methylbut-1-yn-1-yl)-6-(methyl-d₃)-6,7-dihydro-7,14-methanobenzo[f]benzo[4,5]imidazo[1,2-a][1,4]diazocin-5(14H)-one

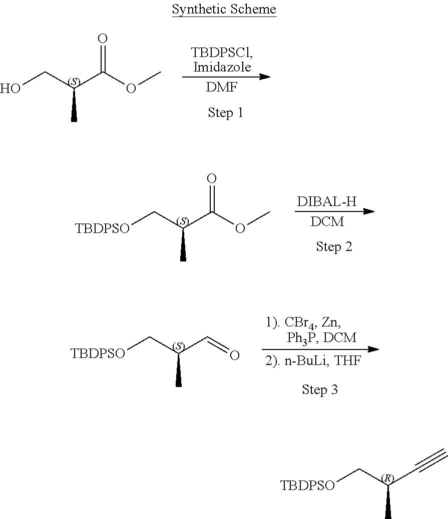

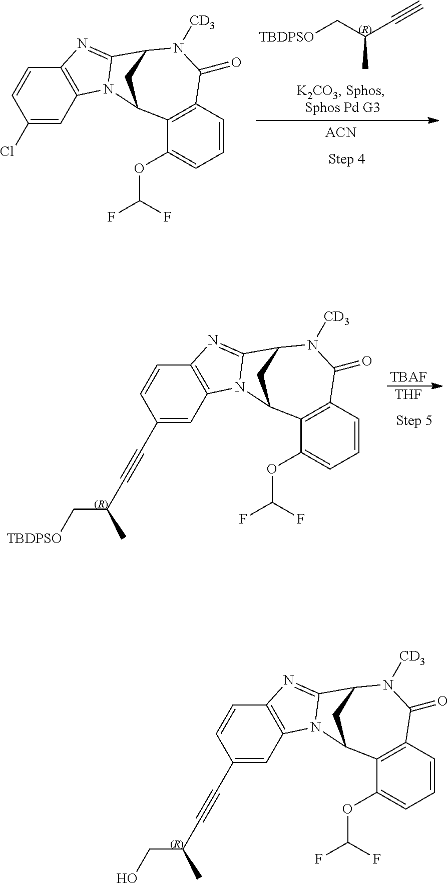

### Preparation 128A: methyl (S)-3-[(tert-butyldiphenylsilyl)oxy]-2-methylpropanoate

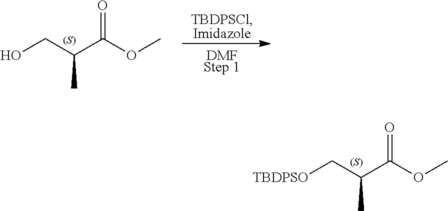

To a stirred solution of methyl (S)-3-hydroxy-2-methylpropanoate (5.00 g, 42.326 mmol) and Imidazole (5.76 g, 84.652 mmol) in DMF (12.5 mL) was added TBDPSCl (11.63 g, 42.326 mmol) dropwise at 0° C. The resulting mixture was stirred for 2 h at room temperature, all the starting material methyl (S)-3-hydroxy-2-methylpropanoate was consumed as evident by TLC analysis. The reaction was quenched by the addition of Water (50 mL) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (10/1) to afford methyl (S)-3-[(tert-butyldiphenylsilyl)oxy]-2-methylpropanoate (10.10 g, 67%) as a colorless oil. MS ESI calculated for C₂₁H₂₈O₃Si [M+H]⁺, 357.18. found N/A. ¹H NMR (400 MHz, Chloroform-d) δ 7.70-7.63 (m, 4H), 7.43-7.35 (m, 6H), 3.85-3.80 (m, 1H), 3.75-3.70 (m, 1H), 3.68 (s, 3H), 2.77-2.66 (m, 1H), 1.15 (d, J=7.1 Hz, 3H), 1.04 (s, 9H).

### Preparation 128B: (S)-3-[(tert-butyldiphenylsilyl)oxy]-2-methylpropanal

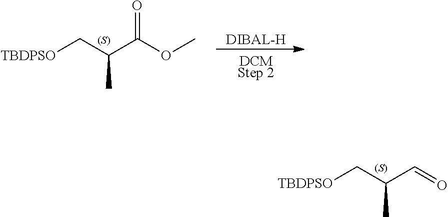

To a stirred solution of methyl (S)-3-[(tert-butyldiphenylsilyl)oxy]-2-methylpropanoate (3.00 g, 8.414 mmol) in DCM (30 mL) was added DIBAL-H in DCM (1.0 M, 9.26 mL, 9.255 mmol) dropwise over 30 min at −78° C. under nitrogen atmosphere. The resulting mixture was stirred for 3 h at −78° C. under nitrogen atmosphere. The reaction was quenched by the addition of MeOH (3 mL) at −70° C. Then the resulting mixture was warmed up to room temperature slowly and poured into sat aq. potassium-sodium tartrate solution (30 mL). The mixture was vigorously stirred at room temperature until phase separation occurred and extracted with CH₂Cl₂ (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (10/1) to afford (S)-3-[(tert-butyldiphenylsilyl)oxy]-2-methylpropanal (1.42 g, 51%) as colorless oil. MS ESI calculated for C₂₀H₂₆O₂Si [M+H]⁺, 327.17. found N/A. ¹H NMR (400 MHz, Chloroform-d) δ 9.79-9.74 (m, 1H), 7.71-7.62 (m, 4H), 7.47-7.33 (m, 6H), 3.95-3.80 (m, 2H), 2.62-2.51 (m, 1H), 1.10 (d, J=7.0 Hz, 3H), 1.04 (s, 9H).

### Preparation 128C: (R)-tert-butyl((2-methylbut-3-yn-1-yl)oxy)diphenylsilane

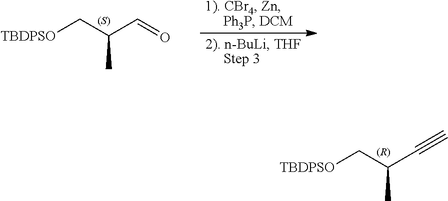

A solution of Zn (0.85 g, 13.047 mmol) and Ph₃P (3.42 g, 13.047 mmol) in DCM (15 mL) was treated with CBr₄ (4.33 g, 13.047 mmol) in DCM (3.0 mL) for 20 min at 0° C. followed by the addition of (S)-3-[(tert-butyldiphenylsilyl)oxy]-2-methylpropanal (1.42 g, 4.349 mmol) in DCM (3.0 mL) dropwise at 0° C. The resulting mixture was stirred for 4 h at room temperature and then it was poured into hexane (100 mL). The resulting mixture was filtered, the filter cake was washed with hexane (3×20 mL). The filtrate was concentrated under reduced pressure. The crude was dissolved in THF (6 mL) and cooled to −78° C. To the above mixture was added n-BuLi in hexanes (2.5M, 4.35 mL, 10.873 mmol) dropwise at −78° C. The resulting mixture was stirred for additional 1 h at −78° C. The reaction was quenched by the addition of sat. NaHCO₃ (aq.) (30 mL) at −78° C. The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE (100%) to afford (R)-tert-butyl((2-methylbut-3-yn-1-yl)oxy)diphenylsilane (480 mg, 34%) as a colorless oil. MS ESI calculated for C₂₁H₂₆OSi [M+H]⁺, 323.18. found N/A. ¹H NMR (300 MHz, Chloroform-d) δ 7.76-7.69 (m, 4H), 7.51-7.39 (m, 6H), 3.83-3.71 (m, 1H), 3.64-3.53 (m, 1H), 2.78-2.63 (m, 1H), 2.07 (d, J=2.4 Hz, 1H), 1.28 (d, J=6.9 Hz, 3H), 1.11 (s, 9H).

### Preparation 128D: (7R,14R)-11-((R)-4-((tert-butyldiphenylsilyl)oxy)-3-methylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

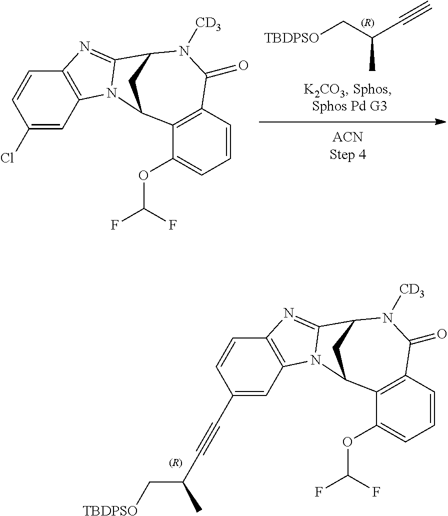

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (140 mg, 0.373 mmol) and (R)-tert-butyl((2-methylbut-3-yn-1-yl)oxy)diphenylsilane (480 mg, 1.492 mmol) in CH₃CN (1.5 mL) was added SPhos (31 mg, 0.075 mmol), SPhos Pd Gen.3 (58 mg, 0.075 mmol) and K₂CO₃ (257 mg, 1.865 mmol) at room temperature under nitrogen atmosphere. The resulting mixture was stirred for 3 h at 80° C. under nitrogen atmosphere. The resulting mixture was concentrated under vacuum. The residue was purified by silica gel column chromatography, eluted with CH₂Cl₂/MeOH (10/1) to afford (7R,14R)-11-((R)-4-((tert-butyldiphenylsilyl)oxy)-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (140 mg, 56%) as a yellow solid. MS ESI calculated for C₄₀H₃₆D₃F₂N₃O₃Si [M+H]⁺, 679.29. found 679.25. ¹H NMR (400 MHz, Chloroform-d) δ 8.52-8.47 (m, 1H), 7.75-7.67 (m, 4H), 7.66-7.60 (m, 1H), 7.52 (s, 1H), 7.50-7.46 (m, 1H), 7.45-7.34 (m, 6H), 7.34-7.30 (m, 2H), 6.92-6.54 (m, 1H), 6.26 (d, J=7.2 Hz, 1H), 5.17 (d, J=7.1 Hz, 1H), 3.83-3.77 (m, 1H), 3.69-3.63 (m, 1H), 3.55-3.43 (m, 1H), 2.94-2.91 (m, 1H), 2.91-2.86 (m, 1H), 1.32 (d, J=6.9 Hz, 3H), 1.08 (s, 9H).

### Example 128: (7R,14R)-1-(difluoromethoxy)-11-((R)-4-hydroxy-3-methylbut-1-yn-1-yl)-6-(methyl-d₃)-6,7-dihydro-7,14-methanobenzo[f]benzo[4,5]imidazo[1,2-a][1,4]diazocin-5(14H)-one

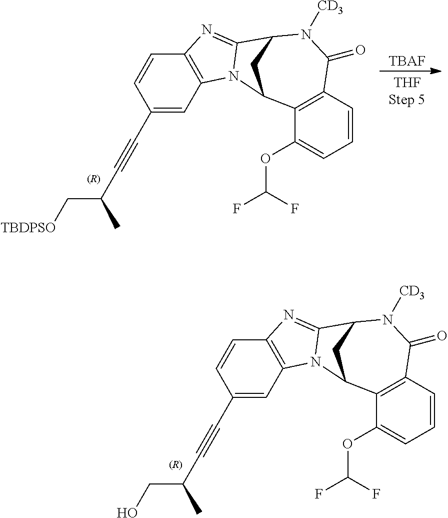

To a stirred solution of (7R,14R)-11-((R)-4-((tert-butyldiphenylsilyl)oxy)-3-methylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (140 mg, 0.206 mmol) in THF (2 mL) was added TBAF in THF (1 M, 0.25 mL, 0.247 mmol) dropwise at 0° C. The resulting mixture was stirred for 1 h at room temperature. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min to afford (7R,14R)-1-(difluoromethoxy)-11-((R)-4-hydroxy-3-methylbut-1-yn-1-yl)-6-(methyl-d₃)-6,7-dihydro-7,14-methanobenzo[f]benzo[4,5]imidazo[1,2-a][1,4]diazocin-5(14H)-one (47 mg, 52%) as a white solid. MS ESI calculated for C₂₄H₁₈D₃F₂N₃O₃ [M+H]⁺, 441.17. found 441.25. ¹H NMR (400 MHz, Chloroform-d) δ 8.50-8.44 (m, 1H), 7.62 (d, J=8.4 Hz, 1H), 7.53-7.50 (m, 1H), 7.43 (t, J=8.2 Hz, 1H), 7.34 (d, J=8.1 Hz, 1H), 7.31-7.28 (m, 1H), 6.86 (t, J=72.2 Hz, 1H), 6.22 (d, J=7.2 Hz, 1H), 5.01 (d, J=7.1 Hz, 1H), 3.71-3.60 (m, 2H), 3.51-3.40 (m, 1H), 2.97-2.89 (m, 1H), 2.87 (d, J=13.6 Hz, 1H), 1.29 (d, J=7.0 Hz, 3H). ¹⁹F NMR (377 MHz, Chloroform-d) δ −80.08, −80.52, −81.28, −81.72.

### Example 129: (7R,14R)-1-(difluoromethoxy)-11-((S)-4-hydroxy-3-methylbut-1-yn-1-yl)-6-(methyl-d₃)-6,7-dihydro-7,14-methanobenzo[f]benzo[4,5]imidazo[1,2-a][1,4]diazocin-5(14H)-one

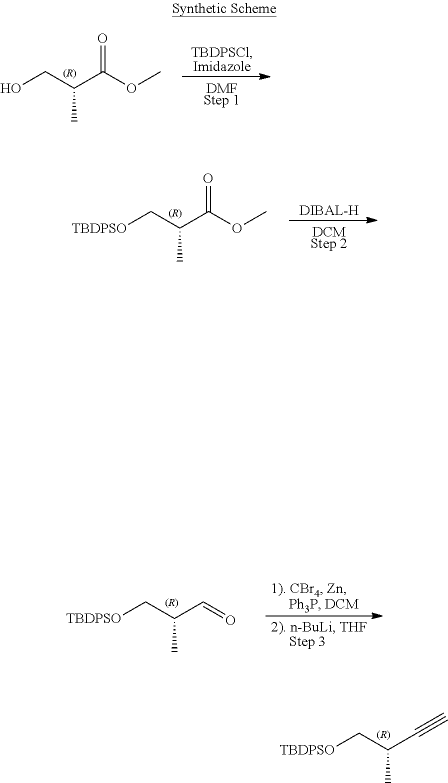

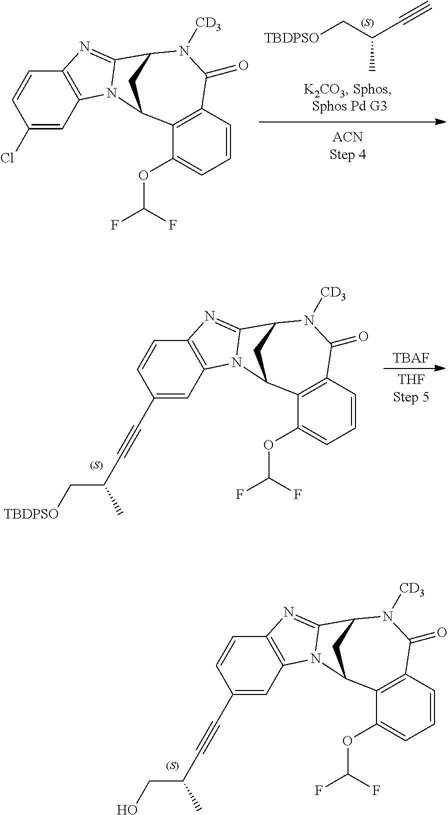

### Preparation 129A: methly (R)-3[(tert-butyldiphenylsilyl)oxy]-2-methylpropanoate

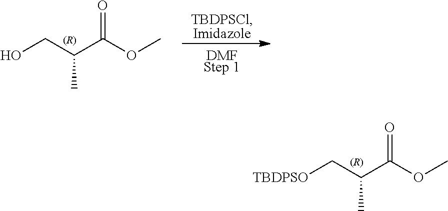

To a stirred solution of methyl (R)-3-hydroxy-2-methylpropanoate (10.00 g, 84.651 mmol) and Imidazole (11.53 g, 169.302 mmol) in DMF (25 mL) was added TBDPSCl (23.27 g, 84.651 mmol) dropwise at 0° C. The resulting mixture was stirred for 2 h at room temperature. The reaction was quenched by the addition of Water (100 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (10/1) to afford methyl (R)-3-[(tert-butyldiphenylsilyl)oxy]-2-methylpropanoate (27.20 g, 90%) as a colorless oil. MS ESI calculated for C₂₁H₂₈O₃Si [M+H]⁺, 357.18, found N/A. ¹H NMR (400 MHz, Chloroform-d) δ 7.70-7.62 (m, 4H), 7.47-7.36 (m, 6H), 3.88-3.81 (m, 1H), 3.77-3.71 (m, 1H), 3.69 (s, 3H), 2.78-2.68 (m, 1H), 1.17 (d, J=7.0 Hz, 3H), 1.04 (s, 9H).

### Preparation 129B: (R)-3-((tert-butyldiphenylsilyl)oxy)-2-methylpropanal

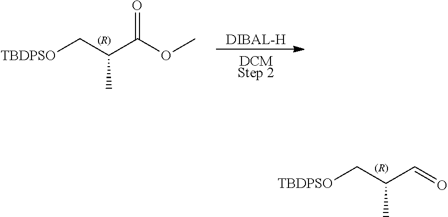

To a stirred solution of methyl (R)-3-[(tert-butyldiphenylsilyl)oxy]-2-methylpropanoate (5.00 g, 14.024 mmol) in DCM (50 mL) was added DIBAL-H in DCM (1.0 M, 15.43 mL, 15.426 mmol) dropwise over 30 min at −78° C. under nitrogen atmosphere. The resulting mixture was stirred for 3 h at −78° C. under nitrogen atmosphere. The reaction was quenched by the addition of MeOH (5 mL) at −70° C. Then the resulting mixture was warmed up to room temperature slowly and poured into sat aq potassium-sodium tartrate solution (50 mL). The mixture was vigorously stirred at room temperature until phase separation occurred and extracted with CH₂Cl₂ (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EA (10/1) to afford (R)-3-[(tert-butyldiphenylsilyl)oxy]-2-methylpropanal (3.27 g, 71%) as colorless oil. MS ESI calculated for C₂₀H₂₆O₂Si [M+H]⁺, 327.17. found N/A. ¹H NMR (300 MHz, Chloroform-d) δ 9.80 (d, J=1.6 Hz, 1H), 7.71-7.65 (m, 4H), 7.51-7.39 (m, 6H), 3.98-3.83 (m, 2H), 2.66-2.53 (m, 1H), 1.13 (d, J=7.0 Hz, 3H), 1.07 (s, 9H).

### Preparation 129C: (S)-tert-butyl((2-methylbut-3-yn-1-yl)oxy)diphenylsilane

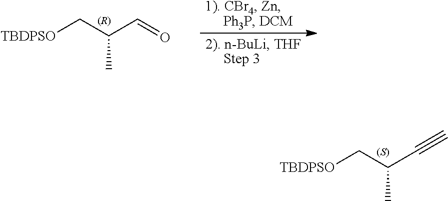

A solution of Zn (300.36 mg, 4.593 mmol) and Ph₃P (1.20 g, 4.593 mmol) in DCM (5 mL) was treated with CBr₄ (1.52 g, 4.593 mmol) in DCM (1.0 mL) for 20 min at 0° C. followed by the addition of (R)-3-[(tert-butyldiphenylsilyl)oxy]-2-methylpropanal (500 mg, 1.531 mmol) in DCM (1.0 mL) dropwise at 0° C. The resulting mixture was stirred for 4 h at room temperature and then it was poured into hexane (30 mL). The resulting mixture was filtered, the filter cake was washed with hexane (3×10 mL). The filtrate was concentrated under reduced pressure. The crude was dissolved in THF (2 mL) and cooled to −78° C. To the above mixture was added n-BuLi in hexanes (2.5 M, 1.53 mL, 3.827 mmol) dropwise at −78° C. The resulting mixture was stirred for additional 1 h at −78° C. The reaction was quenched by the addition of sat. NaHCO₃ (aq.) (10 mL) at −78° C. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with PE (100%) to afford (S)-tert-butyl((2-methylbut-3-yn-1-yl)oxy)diphenylsilane (300 mg, 60%) as a colorless oil. MS ESI calculated for C₂₁H₂₆OSi [M+H]⁺, 323.18. found N/A. ¹H NMR (400 MHz, Chloroform-d) δ 7.71-7.64 (m, 4H), 7.46-7.35 (m, 6H), 3.78-3.67 (m, 1H), 3.60-3.49 (m, 1H), 2.73-2.59 (m, 1H), 2.02 (d, J=2.4 Hz, 1H), 1.23 (d, J=6.9 Hz, 3H), 1.06 (s, 9H).

### Preparation 129D: (7R,14R)-11-((S)-4-((tert-butyldiphenylsilyl)oxy)-3-methylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

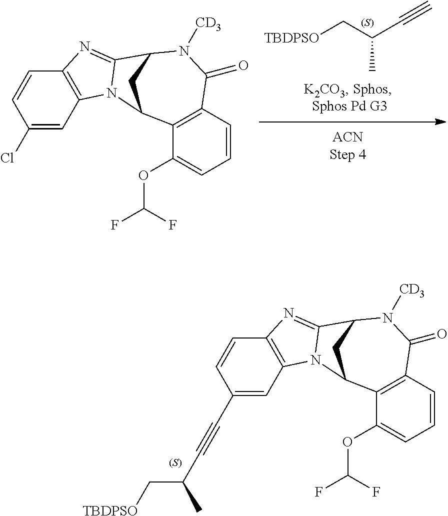

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (120 mg, 0.305 mmol) and (S)-tert-butyl((2-methylbut-3-yn-1-yl)oxy)diphenylsilane (296 mg, 0.915 mmol) in ACN (1 mL) was added SPhos (25 mg, 0.061 mmol), SPhos Pd Gen.3 (48 mg, 0.061 mmol) and K₂CO₃ (211 mg, 1.525 mmol) at room temperature under nitrogen atmosphere. The resulting mixture was stirred for 3 h at 80° C. under nitrogen atmosphere. The resulting mixture was concentrated under vacuum. The residue was purified by silica gel column chromatography, eluted with CH₂Cl₂/MeOH (10/1) to afford (7R,14R)-11-((S)-4-((tert-butyldiphenylsilyl)oxy)-3-methylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (120 mg, 57%) as a yellow solid. MS ESI calculated for C₄₀H₃₆D₃F₂N₃O₃Si [M+H]⁺, 679.29. found 470.00. ¹H NMR (300 MHz, Chloroform-d) δ 8.55-8.46 (m, 1H), 7.77-7.61 (m, 5H), 7.54-7.50 (m, 1H), 7.49-7.30 (m, 9H), 6.72 (t, 1H), 6.25 (d, J=7.1 Hz, 1H), 5.15-5.04 (m, 1H), 3.90-3.78 (m, 1H), 3.72-3.66 (m, 1H), 3.60-3.39 (m, 1H), 2.97-2.91 (m, 1H), 2.89 (d, 1H), 1.34 (d, J=6.9 Hz, 3H), 1.10 (s, 9H).

### Example 129: (7R,14R)-1-(difluoromethoxy)-11-((S)-4-hydroxy-3-methylbut-1-yn-1-yl)-6-(methyl-d₃)-6,7-dihydro-7,14-methanobenzo[f]benzo[4,5]imidazo[1,2-a][1,4]diazocin-5(14H)-one

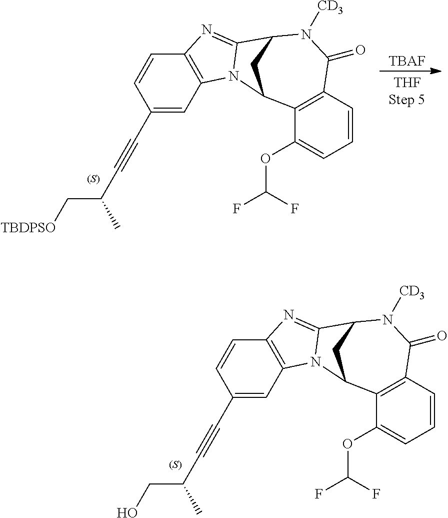

To a stirred solution of (7R,14R)-11-((S)-4-((tert-butyldiphenylsilyl)oxy)-3-methylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (120 mg, 0.177 mmol) in THF (2 mL) was added TBAF in THF (1M, 0.21 mL, 0.212 mmol) dropwise at 0° C. The resulting mixture was stirred for 1 h at room temperature. The resulting mixture was concentrated under vacuum. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min to afford (7R,14R)-1-(difluoromethoxy)-11-((S)-4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (33 mg, 42%) as a white solid. MS ESI calculated for C₂₄H₁₈D₃F₂N₃O₃ [M+H]⁺, 441.17. found 441.15. ¹H NMR (400 MHz, Chloroform-d) δ 8.53-8.45 (m, 1H), 7.62 (d, J=8.5 Hz, 1H), 7.54-7.51 (m, 1H), 7.44 (t, J=8.2 Hz, 1H), 7.35 (d, J=8.2 Hz, 1H), 7.32-7.28 (m, 1H), 6.87 (t, J=72.2 Hz, 1H), 6.24 (d, J=7.2 Hz, 1H), 5.06 (d, J=7.1 Hz, 1H), 3.71-3.60 (m, 2H), 3.52-3.42 (m, 1H), 2.97-2.91 (m, 1H), 2.88 (d, J=13.6 Hz, 1H), 1.29 (d, J=7.0 Hz, 3H). ⁹F NMR (377 MHz, Chloroform-d) δ −80.12, −80.56, −81.32, −81.77.

### Example 130: (7R,14R)-1-(difluoromethoxy)-11-((1-(hydroxymethyl)cyclopropyl)ethynyl)-6-(methyl-d₃)-6,7-dihydro-7,14-methanobenzo[f]benzo[4,5]imidazo[1,2-a][1,4]diazocin-5(14H)-one

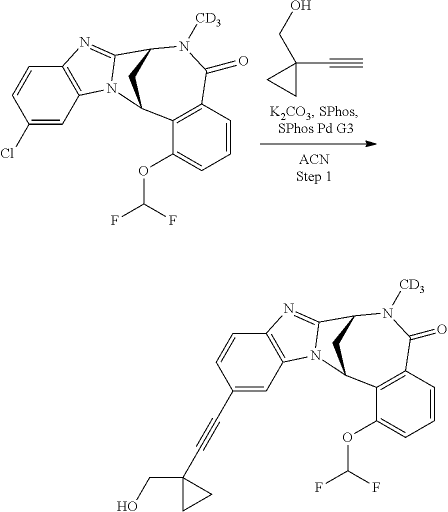

A mixture of (7R,14R)-11-chloro-1-(difluoromethoxy)-6-(methyl-d₃)-6,7-dihydro-7,14-methanobenzo[f]benzo[4,5]imidazo[1,2-a][1,4]diazocin-5(14H)-one (60 mg, 0.153 mmol), K₂CO₃ (106 mg, 0.765 mmol), SPhos (13 mg, 0.031 mmol), SPhos Pd Gen.3 (24 mg, 0.031 mmol) and (1-ethynylcyclopropyl)methanol (73 mg, 0.765 mmol) in CH₃CN (1 mL) was stirred for 2 h at 80° C. under nitrogen atmosphere. The resulting mixture was concentrated under reduced pressure. The residue was purified by silica gel column chromatography, eluted with CH₂Cl₂/MeOH (10/1) followed by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: CH₃CN; Flow rate: 60 mL/min; Gradient: 23% B to 40% B in 10 min; Wave Length: 254/220 nm; RT1(min): 12.23) to afford (7R,14R)-1-(difluoromethoxy)-11-((1-(hydroxymethyl)cyclopropyl)ethynyl)-6-(methyl-d₃)-6,7-dihydro-7,14-methanobenzo[f]benzo[4,5]imidazo[1,2-a][1,4]diazocin-5(14H)-one (21 mg, 31%) as a white solid. MS ESI calculated for C₂₅H₁₈D₃F₂N₃O₃ [M+H]⁺, 453.17. found 453.05. ¹H NMR (400 MHz, Chloroform-d) δ 8.50-8.43 (m, 1H), 7.61-7.55 (m, 1H), 7.49 (s, 1H), 7.42 (t, J=8.2 Hz, 1H), 7.37-7.33 (m, 1H), 7.26-7.23 (m, 1H), 6.86 (t, J=72.1 Hz, 1H), 6.20 (d, J=7.2 Hz, 1H), 5.00 (d, J=7.1 Hz, 1H), 3.59 (s, 2H), 3.50-3.38 (m, 1H), 2.85 (d, J=13.6 Hz, 1H), 1.13-1.04 (m, 2H), 0.92-0.84 (m, 2H). ¹⁹F NMR (377 MHz, Chloroform-d) δ −79.99, −80.44, −81.37, −81.81.

### Example 131: (7R,14R)-1-(difluoromethoxy)-11-(3-(difluoromethoxy)prop-1-yn-1-yl)-6-(methyl-d₃)-6,7-dihydro-7,14-methanobenzo[f]benzo[4,5]imidazo[1,2-a][1,4]diazocin-5(14H)-one

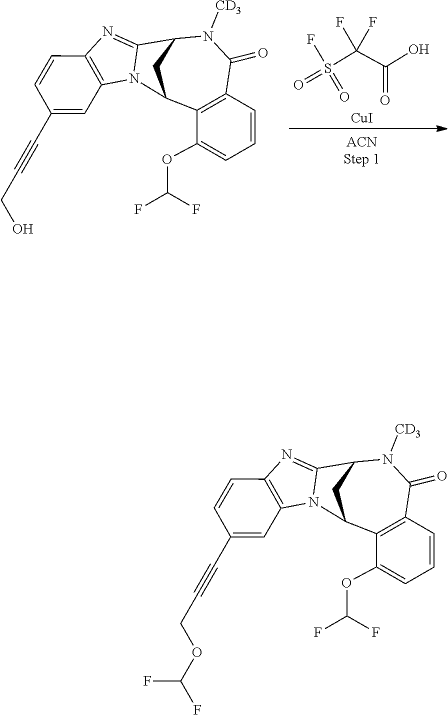

To a stirred solution of (7R,14R)-1-(difluoromethoxy)-11-(3-hydroxyprop-1-yn-1-yl)-6-(methyl-d₃)-6,7-dihydro-7,14-methanobenzo[f]benzo[4,5]imidazo[1,2-a][1,4]diazocin-5(14H)-one (55 mg, 0.133 mmol) and CuI (5 mg, 0.027 mmol) in CH₃CN (1 mL) was added 2,2-difluoro-2-(fluorosulfonyl)acetic acid (28 uL, 0.266 mmol) at room temperature. The resulting mixture was stirred for 2 h at 50° C. under nitrogen atmosphere. The reaction was quenched with Na₂CO₃(aq.) at room temperature. The resulting mixture was extracted with EtOAc (3×4 mL). The combined organic layers was washed with brine (3×6 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 30% to 50% gradient in 20 min to afford (7R,14R)-1-(difluoromethoxy)-11-(3-(difluoromethoxy)prop-1-yn-1-yl)-6-(methyl-d₃)-6,7-dihydro-7,14-methanobenzo[f]benzo[4,5]imidazo[1,2-a][1,4]diazocin-5(14H)-one (8 mg, 13%) as a white solid. MS ESI calculated for C₂₃H₁₄D₃F₄N₃O₃ [M+H]⁺, 463.14. found 463.10. ¹H NMR (400 MHz, DMSO-d₆) δ 8.29-8.25 (m, 1H), 7.75-7.40 (m, 5H), 7.31-7.27 (m, 1H), 6.79 (t, J=76 Hz, 1H), 6.24 (d, J=7.2 Hz, 1H), 5.24 (d, J=7.1 Hz, 1H), 4.86 (s, 2H), 3.54-3.48 (m, 1H), 2.81 (d, J=13.7 Hz, 1H). ¹⁹F NMR (377 MHz, DMSO-d) δ −82.02, −82.05, −83.05.

### Example 132: (7R,14R)-1-(difluoromethoxy)-11-(5,5-difluoropent-1-yn-1-yl)-6-(methyl-d₃)-6,7-dihydro-7,14-methanobenzo[f]benzo[4,5]imidazo[1,2-a][1,4]diazocin-5(14H)-one

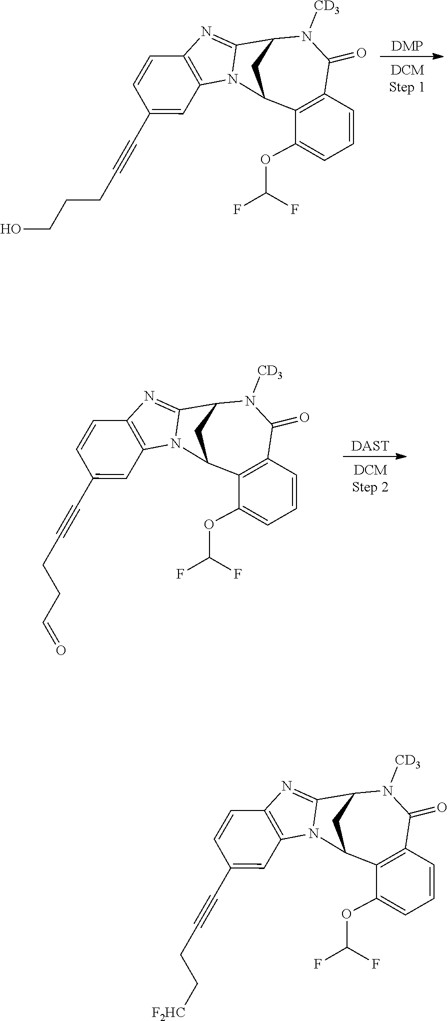

### Preparation 132A: 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynal

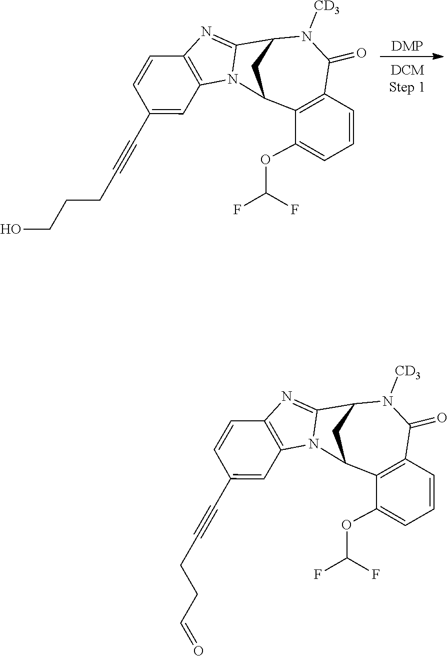

To a stirred solution of (7R,14R)-1-(difluoromethoxy)-11-(5-hydroxypent-1-yn-1-yl)-6-(methyl-d₃)-6,7-dihydro-7,14-methanobenzo[f]benzo[4,5]imidazo[1,2-a][1,4]diazocin-5(14H)-one (100 mg, 0.227 mmol) in DCM (1 mL) was added DMP (116 mg, 0.272 mmol) at 0° C. under nitrogen atmosphere. The resulting mixture was stirred for 2 h at room temperature under nitrogen atmosphere. The reaction was quenched with sat. NaHCO₃ (aq.) at room temperature. The resulting mixture was extracted with CH₂Cl₂ (3×4 mL). The combined organic layers were washed with Na₂S₂O₃(4 mL), followed brine (1×6 mL), dried over anhydrous Na₂SO₄. After filtration, the filtrate was concentrated under reduced pressure. The residue was purified by silica gel column chromatography, eluted with CH₂Cl₂/MeOH (20/1) to afford 5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-ynal (99 mg, 99%) as a white solid. MS ESI calculated for C₂₄H₁₆D₃F₂N₃O₃ [M−H]⁻, 437.16. found 437.10. ¹H NMR (400 MHz, Chloroform-d) δ 9.87 (d, J=1.2 Hz, 1H), 8.50 (d, J=8.2 Hz, 1H), 7.67 (d, J=8.6 Hz, 1H), 7.53 (s, 1H), 7.49-7.44 (m, 1H), 7.35 (t, J=9.3 Hz, 2H), 6.87 (t, J=72 Hz, 1H), 6.28 (d, J=7.0 Hz, 1H), 5.18 (d, J=6.9 Hz, 1H), 3.53-3.45 (m, 1H), 2.91 (d, J=13.6 Hz, 1H), 2.84-2.73 (m, 4H).

### Example 132: (7R,14R)-1-(difluoromethoxy)-11-(5,5-difluoropent-1-yn-1-yl)-6-(methyl-d₃)-6,7-dihydro-7,14-methanobenzo[f]benzo[4,5]imidazo[1,2-a][1,4]diazocin-5(14H)-one

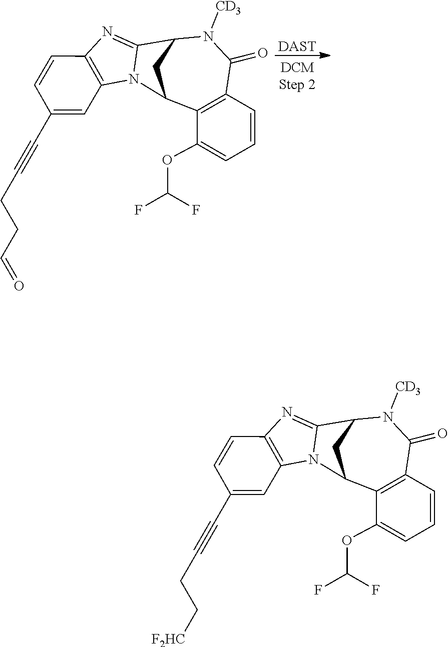

To a stirred solution of 5-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)pent-4-ynal (99 mg, 0.226 mmol) in DCM (1 mL) was added N,N-diethyl-1,1,1-trifluoro-14-sulfanamine (45 uL, 0.339 mmol) dropwise at 0° C. under nitrogen atmosphere. The resulting mixture was stirred for 2 h at room temperature under nitrogen atmosphere. The reaction was concentrated under reduce pressure. The residue was purified by Prep-TLC (CH₂Cl₂/MeOH=20/1) followed by reversed-phase flash chromatography with the following conditions: column, C18 silica gel; mobile phase, CH₃CN in Water (10 mmol/L NH₄HCO₃), 30% to 50% gradient in 20 min to afford (7R,14R)-1-(difluoromethoxy)-11-(5,5-difluoropent-1-yn-1-yl)-6-(methyl-d3)-6,7-dihydro-7,14-methanobenzo[f]benzo[4,5]imidazo[1,2-a][1,4]diazocin-5(14H)-one (23 mg, 21%) as a white solid. MS ESI calculated for C₂₄H₁₆D₃F₄N₃O₂ [M+H]⁺, 461.16. found 461.15. ¹H NMR (400 MHz, DMSO-d₆) δ 8.31-8.25 (m, 1H), 7.59 (d, J=2.2 Hz, 1H), 7.57 (t, J=74 Hz, 1H), 7.54-7.47 (m, 2H), 7.45 (s, 1H), 7.24-7.18 (m, 1H), 6.35-5.99 (m, 2H), 5.21 (d, J=7.1 Hz, 1H), 3.56-3.47 (m, 1H), 2.80 (d, J=13.8 Hz, 1H), 2.59 (t, J=7.3 Hz, 2H), 2.20-2.04 (m, 2H). ¹⁹F NMR (377 MHz, DMSO-d₆) δ −82.12, −82.17, −116.73

### Example 133: (7R,14R)-1-(difluoromethoxy)-6-(methyl-d₃)-11-(5-(4-methylpiperazin-1-yl)pent-1-yn-1-yl)-6,7-dihydro-7,14-methanobenzo[f]benzo[4,5]imidazo[1,2-a][1,4]diazocin-5(14H)-one

### Synthetic Scheme

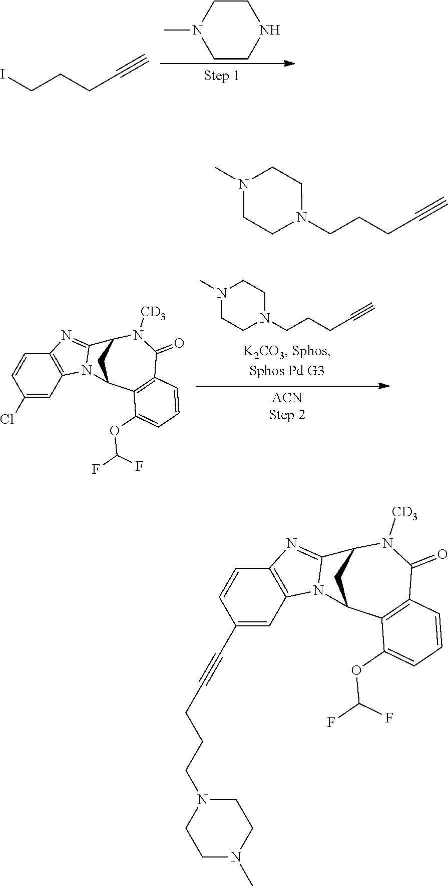

### Preparation 133A: 1-methyl-4-(pent-4-yn-1-yl)piperazine

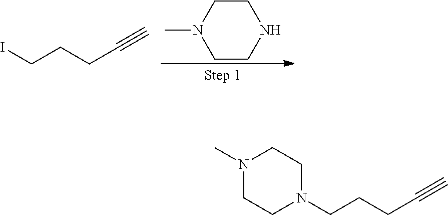

A mixture of 5-iodopent-1-yne (500 mg, 2.577 mmol) and piperazine and 1-methyl- (645 mg, 6.442 mmol) was stirred for 15 min at 80° C. The resulting mixture was diluted with ethyl ether (20 mL). The precipitated solids were collected by filtration and washed with ethyl ether (2×5 mL). The resulting mixture was washed with 3×30 mL of HCl (2 N). Then, the aqueous layer was basified to pH 9 with NaOH (2 N). The resulting mixture was extracted with EtOAc (3×100 mL). The combined organic layers were washed with brine (1×100 mL), dried over anhydrous Na₂SO₄. After filtration, the filtrate was concentrated under reduced pressure. This resulted in 1-methyl-4-(pent-4-yn-1-yl)piperazine (300 mg, 70%) as a yellow oil. MS ESI calculated for C₁₀H₁₈N₂ [M+H]⁺, 167.15. found N/A. ¹H NMR (400 MHz, Chloroform-d) δ 2.58-2.40 (m, 8H), 2.31 (s, 3H), 2.28-2.17 (m, 4H), 1.98-1.93 (m, 1H), 1.80-1.66 (m, 2H).

### Example 133: (7R,14R)-1-(difluoromethoxy)-6-(methyl-d₃)-11-(5-(4-methylpiperazin-1-yl)pent-1-yn-1-yl)-6,7-dihydro-7,14-methanobenzo[f]benzo[4,5]imidazo[1,2-a][1,4]diazocin-5(14H)-one

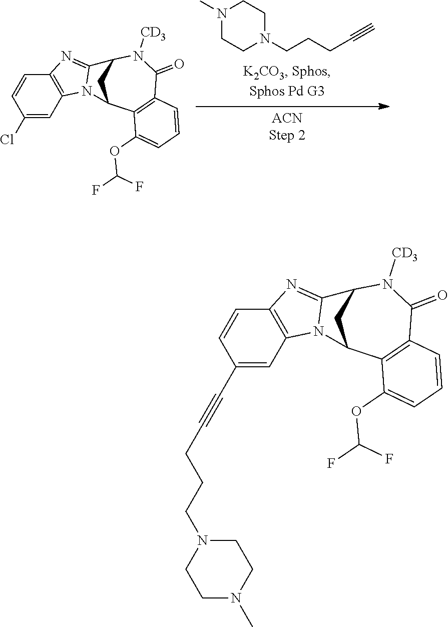 [SMILES: [2H]C([2H])([2H])N1C(=O)c2cccc(OC(F)F)c2[C@H]2C[C@@H]1c1nc3ccc(Cl)cc3n12 ; C#CCCCN1CCN(C)CC1 ; [2H]C([2H])([2H])N1C(=O)c2cccc(OC(F)F)c2[C@H]2C[C@@H]1c1nc3ccc(C#CCCCN4CCN(C)CC4)cc3n12]

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and 1-methyl-4-(pent-4-yn-1-yl)piperazine (212 mg, 1.275 mmol) in ACN (2 mL) were added SPhos Pd Gen.3 (40 mg, 0.051 mmol), SPhos (21 mg, 0.051 mmol) and K₂CO₃ (176 mg, 1.275 mmol) at room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min. This resulted in (7R,14R)-1-(difluoromethoxy)-6-(methyl-d₃)-11-(5-(4-methylpiperazin-1-yl)pent-1-yn-1-yl)-6,7-dihydro-7,14-methanobenzo[f]benzo[4,5]imidazo[1,2-a][1,4]diazocin-5(14H)-one (5 mg, 3%) as a white solid. MS ESI calculated for C₂₉H₂₈D₃F₂N₅O₂ [M+H]⁺, 523.26. found 523.25. ¹H NMR (400 MHz, Chloroform-d) δ 8.48 (d, J=8.1 Hz, 1H), 7.64-7.56 (m, 1H), 7.48 (s, 1H), 7.44-7.38 (m, 1H), 7.35-7.28 (m, 1H), 7.26-7.22 (m, 1H), 7.00-6.66 (m, 1H), 6.20 (d, J=7.3 Hz, 1H), 4.92 (d, J=7.1 Hz, 1H), 3.47-3.37 (m, 1H), 2.84 (d, J=13.6 Hz, 1H), 2.66-2.41 (m, 10H), 2.35 (s, 3H), 1.89-1.77 (m, 4H). ¹⁹F NMR (377 MHz, Chloroform-d) δ −79.92, −80.37, −81.03, −81.48.

### Example 134: (7R,14R)-1-(difluoromethoxy)-6-(methyl-d₃)-11-(4-(4-methylpiperazin-1-yl)but-1-yn-1-yl)-6,7-dihydro-7,14-methanobenzo[f]benzo[4,5]imidazo[1,2-a][1,4]diazocin-5(14H)-one

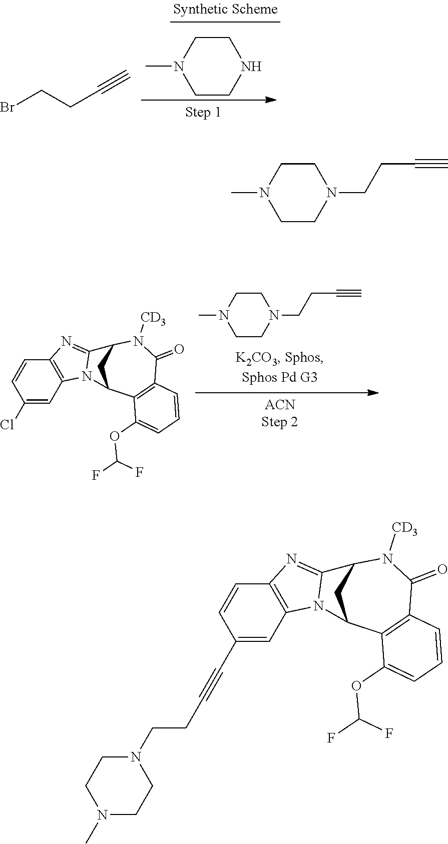

### Preparation 134A: 1-(but-3-yn-yl)-4-methylpiperazine

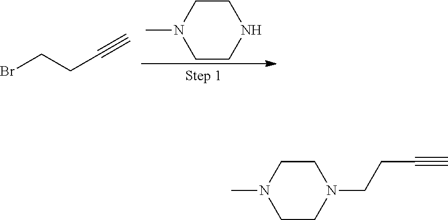

A mixture of 4-bromobut-1-yne (500 mg, 3.760 mmol) and 1-methylpiperazine (941 mg, 9.400 mmol) was stirred for 15 min at 80° C. The resulting mixture was diluted with ethyl ether (20 mL). The precipitated solids were collected by filtration and washed with ethyl ether (2×5 mL). The resulting mixture was washed with 3×30 mL of HCl (2 N). Then, the aqueous layer was basified to pH 9 with NaOH (2 N). The resulting mixture was extracted with EtOAc (3×100 mL). The combined organic layers were washed with brine (1×100 mL), dried over anhydrous Na₂SO₄. After filtration, the filtrate was concentrated under reduced pressure. This resulted in 1-(but-3-yn-1-yl)-4-methylpiperazine (268 mg, 47%) as a yellow oil. MS ESI calculated for C₉H₁₆N₂[M+H]⁺, 153.13. found N/A. ¹H NMR (400 MHz, Chloroform-d) δ 2.66-2.58 (m, 3H), 2.56-2.53 (m, 2H), 2.51-2.47 (m, 2H), 2.44-2.35 (m, 3H), 2.31 (s, 3H), 2.18-2.11 (m, 2H), 2.01-1.97 (m, 1H).

### Example 134: (7R,14R)-1-(difluoromethoxy)-6-(methyl-d₃)-11-(4-(4-methylpiperazin-1-yl)but-1-yn-1-yl)-6,7-dihydro-7,14-methanobenzo[f]benzo[4,5]imidazo[1,2-a][1,4]diazocin-5(14H)-one

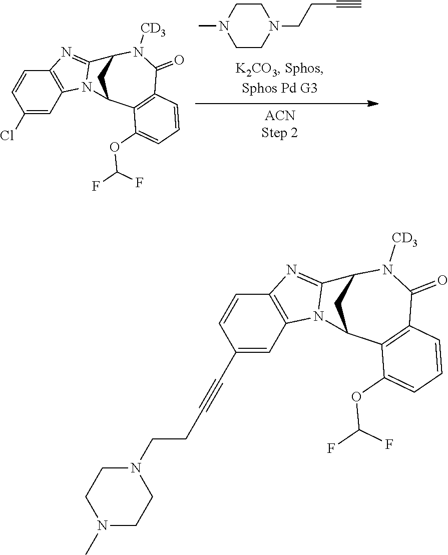

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and 1-(but-3-yn-1-yl)-4-methylpiperazine (194 mg, 1.275 mmol) in ACN (3 mL) were added SPhos Pd Gen.3 (40 mg, 0.051 mmol), SPhos (21 mg, 0.051 mmol) and K₂CO₃ (176 mg, 1.275 mmol) in portions at room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min. This resulted in (7R,14R)-1-(difluoromethoxy)-6-(methyl-d₃)-11-(4-(4-methylpiperazin-1-yl)but-1-yn-1-yl)-6,7-dihydro-7,14-methanobenzo[f]benzo[4,5]imidazo[1,2-a][1,4]diazocin-5(14H)-one (42 mg, 32%) as a white solid. MS ESI calculated for C₂₈H₂₆D₃F₂N₅O₂ [M+H]⁺, 509.25. found 509.25. ¹H NMR (400 MHz, Chloroform-d) δ 8.53-8.44 (m, 1H), 7.65-7.56 (m, 1H), 7.51-7.46 (m, 1H), 7.41 (t, J=8.2 Hz, 1H), 7.37-7.32 (m, 1H), 7.26-7.22 (m, 1H), 6.85 (t, J=72.2 Hz, 1H), 6.19 (d, J=7.2 Hz, 1H), 4.92 (d, J=7.1 Hz, 1H), 3.52-3.32 (m, 1H), 2.84 (d, J=13.5 Hz, 1H), 2.76-2.68 (m, 3H), 2.68-2.43 (m, 9H), 2.34 (s, 3H). ¹⁹F NMR (377 MHz, Chloroform-d) δ −79.85, −80.29, −81.04, −81.49.

### Example 135: (7R,14R)-11-(4-(azetidin-1-yl)but-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

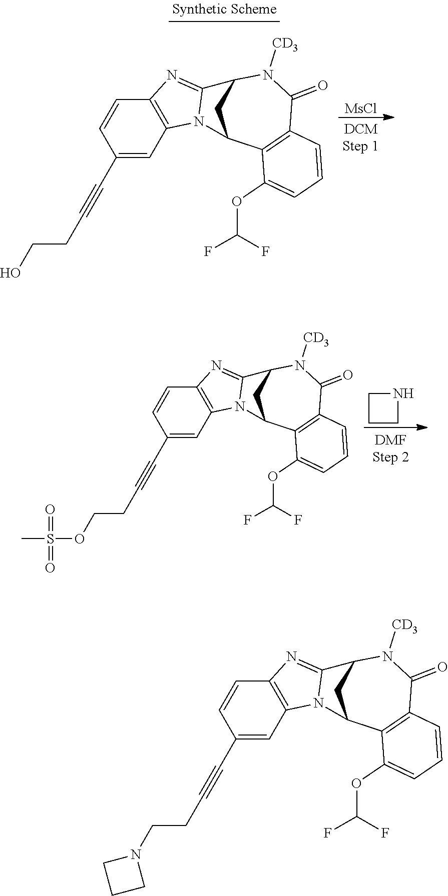

### Preparation 135A: 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin--yl)but-3-yn-1-yl methanesulfonate

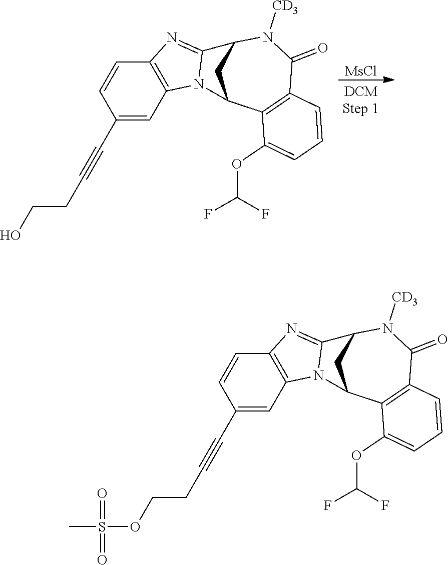 [SMILES: "MsCl" ; [2H]C([2H])([2H])N1C(=O)c2cccc(OC(F)F)c2[C@H]2C[C@@H]1c1nc3ccc(C#CCCO)cc3n12 ; [2H]C([2H])([2H])N1C(=O)c2cccc(OC(F)F)c2[C@H]2C[C@@H]1c1nc3ccc(C#CCCOS(C)(=O)=O)cc3n12]

To a stirred solution of (7R,14R)-1-(difluoromethoxy)-11-(4-hydroxybut-1-yn-1-yl)-6-(methyl-d₃)-6,7-dihydro-7,14-methanobenzo[f]benzo[4,5]imidazo[1,2-a][1,4]diazocin-5(14H)-one (150 mg, 0.352 mmol) in DCM (2 mL) was added TEA (98 uL, 0.704 mmol) at 0° C. under nitrogen atmosphere. To the above mixture was added MsCl (41 uL, 0.528 mmol). The resulting mixture was stirred for additional 1 h at room temperature. The reaction was quenched with Water at room temperature. The resulting mixture was extracted with CH₂Cl₂ (3×3 mL). The combined organic layers were washed with brine (5 mL), dried over anhydrous Na₂SO₄. After filtration, the filtrate was concentrated under reduced pressure to afford 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl methanesulfonate (175 mg, 98%) as a yellow solid. MS ESI calculated for C₂₄H₁₈D₃F₂N₃O₅S [M+H]⁺, 505.14 found 505.20. ¹H NMR (300 MHz, Chloroform-d) δ 8.54-8.47 (m, 1H), 8.16-7.52 (m, 2H), 7.51-7.43 (m, 1H), 7.41-7.31 (m, 2H), 6.90 (t, J=78.4 Hz, 1H), 6.29 (d, J=7.0 Hz, 1H), 5.21-5.07 (m, 1H), 4.43 (t, J=6.7 Hz, 2H), 3.60-3.46 (m, 1H), 3.09 (s, 3H), 2.97-2.87 (m, 3H).

### Example 135: (7R,14R)-11-(4-(azetidin-1-yl)but-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

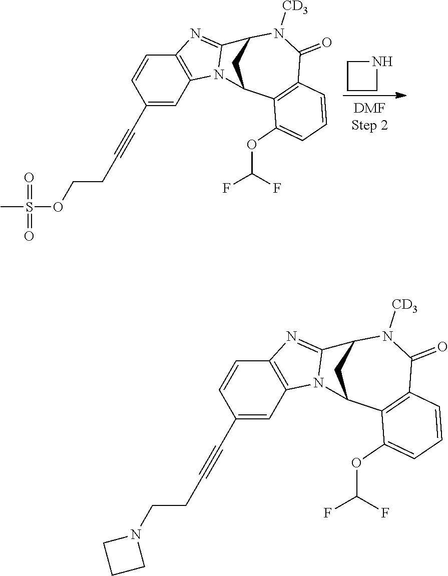

A solution of 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl methanesulfonate (30 mg, 0.059 mmol) and azetidine (34 mg, 0.590 mmol) in DMF (0.5 mL) was stirred for overnight at 50° C. under nitrogen atmosphere. The residue was purified by reversed-phase flash chromatography with the following conditions: column, C18 silica gel; mobile phase, CH₃CN in Water (10 mmol/L NH₄HCO₃), 30% to 80% gradient in 25 min to afford (7R,14R)-11-(4-(azetidin-1-yl)but-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (5 mg, 17%) as a white solid. MS ESI calculated for C₂₆H₂₁D₃F₂N₄O₂ [M+H]⁺, 466.21. found 466.25. ¹H NMR (400 MHz, Methanol-d4) δ 8.41-8.31 (m, 1H), 7.59-7.34 (m, 4H), 7.26-7.23 (m, 1H), 7.23 (t, J=78.4 Hz, 1H), 6.37-6.27 (m, 1H), 5.21-5.10 (m, 1H), 3.57-3.46 (m, 1H), 3.40-3.33 (m, 4H), 2.90-2.82 (m, 1H), 2.73-2.65 (m, 2H), 2.51-2.44 (m, 2H), 2.18-2.08 (m, 2H). ¹⁹F NMR (377 MHz, Methanol-d₄) δ −82.89, −83.34, −83.49, −83.94.

### Example 136: (7R,14R)-1-(difluoromethoxy)-6-(methyl-d₃)-11-(4-(pyrrolidin-1-yl)but-1-yn-1-yl)-6,7-dihydro-7,14-methanobenzo[f]benzo[4,5]imidazo[1,2-a][1,4]diazocin-5(14H)-one

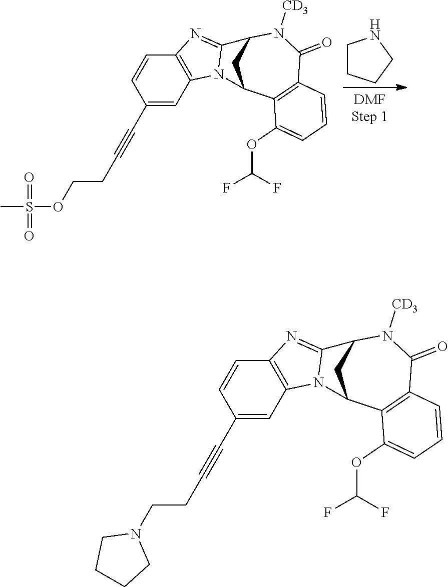

A solution of 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-yn-1-yl methanesulfonate (25 mg, 0.050 mmol) and pyrrolidine (35 mg, 0.500 mmol) in DMF (0.5 mL) was stirred for 16 h at 50° C. under nitrogen atmosphere. The residue was purified by reversed-phase flash chromatography with the following conditions: column, C18 silica gel; mobile phase, CH₃CN in Water (10 mmol/L NH₄HCO₃), 30% to 85% gradient in 25 min to afford (7R,14R)-1-(difluoromethoxy)-6-(methyl-d₃)-11-(4-(pyrrolidin-1-yl)but-1-yn-1-yl)-6,7-dihydro-7,14-methanobenzo[f]benzo[4,5]imidazo[1,2-a][1,4]diazocin-5(14H)-one (6 mg, 25%) as a white solid. MS ESI calculated for C₂₇H₂₃D₃F₂N₄O₂ [M+H]⁺, 480.22. found 480.20. ¹H NMR (400 MHz, Methanol-d4) δ 8.41-8.31 (m, 1H), 7.58-7.42 (m, 4H), 7.27-7.24 (m, 1H), 7.23 (t, J=78.4 Hz, 1H), 6.34 (d, J=7.1 Hz, 1H), 5.16 (d, J=7.1 Hz, 1H), 3.59-3.46 (m, 1H), 2.86 (d, J=13.9 Hz, 1H), 2.79-2.73 (m, 2H), 2.69-2.59 (m, 6H), 1.88-1.77 (m, 4H). ¹⁹F NMR (377 MHz, Methanol-d₄) δ −82.98, −83.43, −83.49, −83.94.

### Example 137: (7R,14R)-11-(4-cyclopropylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

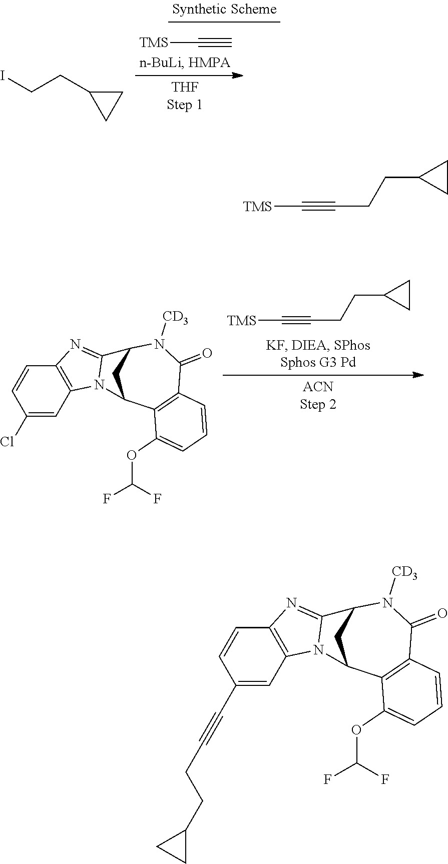

### Preparation 137A: (4-cyclopropylbut-1-yn-1-yl)trimethylsilane

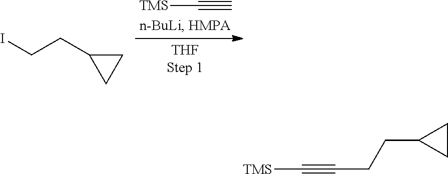

To a stirred mixture of trimethylsilylacetylene (501 mg, 5.101 mmol) in THF (10 mL) was added n-BuLi in hexane (2.5 M, 2.7 mL, 6.631 mmol) at −78° C. under nitrogen atmosphere. The resulting mixture was stirred for 1 h at −78° C. under nitrogen atmosphere. To the above mixture was added HMPA (1.37 g, 7.652 mmol) at −78° C. The resulting mixture was stirred for additional 30 min at −78° C. To the above mixture was added (2-iodoethyl)cyclopropane (1.00 g, 5.101 mmol) at −78° C. The resulting mixture was stirred for additional 16 h at room temperature. The reaction was quenched with water at room temperature. The resulting mixture was diluted with PE (50 mL). The residue was washed with water (3×50 mL). The resulting mixture was concentrated under vacuum. The residue was purified by silica gel column chromatography, eluted with PE to afford (4-cyclopropylbut-1-yn-1-yl)trimethylsilane (330 mg, 39%) as a colorless oil. MS ESI calculated for C₁₀H₁₈Si [M+H]⁺, 167.12. found N/A. ¹H NMR (400 MHz, Chloroform-d) δ 2.31 (t, J=7.3 Hz, 2H), 1.47-1.39 (m, 2H), 0.84-0.74 (m, 1H), 0.49-0.39 (m, 2H), 0.14 (s, 9H), 0.09-0.04 (m, 2H).

### Example 137: (7R,14R)-11-(4-cyclopropylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one

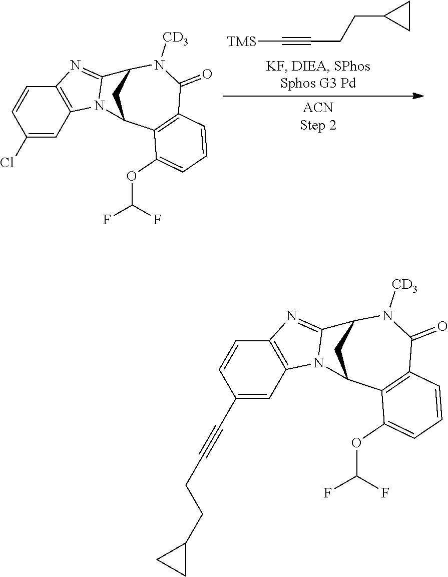 [SMILES: [2H]C([2H])([2H])N1C(=O)c2cccc(OC(F)F)c2[C@H]2C[C@@H]1c1nc3ccc(Cl)cc3n12 ; C[Si](C)(C)C#CCCC1CC1 ; [2H]C([2H])([2H])N1C(=O)c2cccc(OC(F)F)c2[C@H]2C[C@@H]1c1nc3ccc(C#CCCC4CC4)cc3n12]

To a stirred mixture of (7R,14R)-11-chloro-1-(difluoromethoxy)-6-(methyl-d₃)-6,7-dihydro-7,14-methanobenzo[f]benzo[4,5]imidazo[1,2-a][1,4]diazocin-5(14H)-one (50 mg, 0.127 mmol) and (4-cyclopropylbut-1-yn-1-yl)trimethylsilane (64 mg, 0.381 mmol) in ACN (1.5 mL) were added KF (15 mg, 0.254 mmol), DIEA (0.11 mL, 0.635 mmol), SPhos (10 mg, 0.025 mmol) and SPhos Pd Gen.3 (20 mg, 0.025 mmol) at room temperature. The resulting mixture was stirred for 3 h at 80° C. under nitrogen atmosphere. The resulting mixture was concentrated under vacuum. The residue was purified by silica gel column chromatography, eluted with CH₂Cl₂/MeOH (20/1) to afford the crude product. The mixture (20 mg) was resolved by Chiral HPLC with the following conditions: Column: CHIRALPAK IG, 3*25 cm, 5 μm; Mobile Phase A: Hex (10 mM NH₃-MeOH), Mobile Phase B: EtOH; Flow rate: 40 mL/min; Gradient: isocratic 30; Wave Length: 202/225 nm; RT1(min): 10.1; RT2(min): 15.2. The first peak afforded (7R,14R)-11-(4-cyclopropylbut-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (3 mg, 4%) as a white solid. MS ESI calculated for C₂₆H₂₀D₃F₂N₃O₂[M+H]⁺, 451.19. found 451.15. ¹H NMR (400 MHz, Chloroform-d) δ 8.51-8.45 (m, 1H), 7.63 (d, J=8.4 Hz, 1H), 7.51 (s, 1H), 7.47-7.40 (m, 1H), 7.37-7.29 (m, 2H), 6.74 (t, J=72.5 Hz, 1H), 6.27-6.17 (m, 1H), 5.07-4.98 (m, 1H), 3.55-3.41 (m, 1H), 2.87 (d, J=10.0 Hz, 1H), 2.57-2.45 (m, 2H), 1.58-1.47 (m, 2H), 0.92-0.81 (m, 1H), 0.55-0.43 (m, 2H), 0.18-0.08 (m, 2H). ¹⁹F NMR (377 MHz, Chloroform-d) δ −79.95, −80.39, −81.13, −81.57.

### Example 138: methyl 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-ynoate

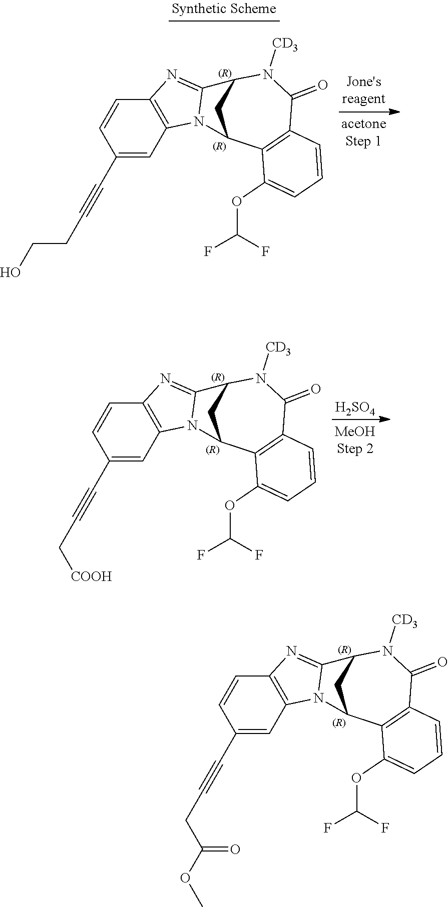

### Preparation 138A: 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-ynoic acid

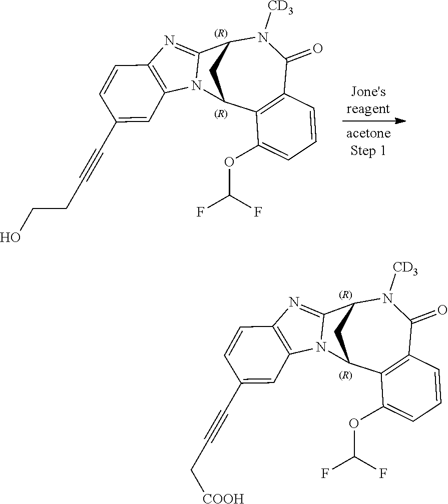

To a stirred solution of (7R,14R)-1-(difluoromethoxy)-11-(4-hydroxybut-1-yn-1-yl)-6-(methyl-d₃)-6,7-dihydro-7,14-methanobenzo[f]benzo[4,5]imidazo[1,2-a][1,4]diazocin-5(14H)-one (100 mg, 0.235 mmol) in acetone (1 mL) was added Jones' reagent (0.2 mL, 0.470 mmol) dropwise at 0° C. under nitrogen atmosphere. The resulting mixture was stirred for 3 h at 0° C. under nitrogen atmosphere. The reaction was quenched by the addition of i-PrOH (2 mL) at 0° C. The resulting mixture was filtered, the filter cake was washed with DCM (2×2 mL). The resulting mixture was extracted with CH₂Cl₂ (2×3 mL). The combined organic layers were washed with brine (1×4 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 30% to 50% gradient in 20 min to afford 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-ynoic acid (85 mg, 82%) as a white solid. MS ESI calculated for C₂₃H₁₄D₃F₂N₃O₄[M+H]⁺, 441.14. found 440.20. ¹H NMR (400 MHz, DMSO-d₆) δ 13.49-12.04 (m, 1H), 8.30-8.24 (m, 1H), 7.76-7.33 (m, 5H) 7.25-7.17 (m, 1H), 6.22 (d, J=7.0 Hz, 1H), 5.24-5.16 (m, 1H), 3.57 (s, 2H), 3.54-3.47 (m, 1H), 2.80 (d, J=13.7 Hz, 1H).

### Example 138: methyl 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-ynoate

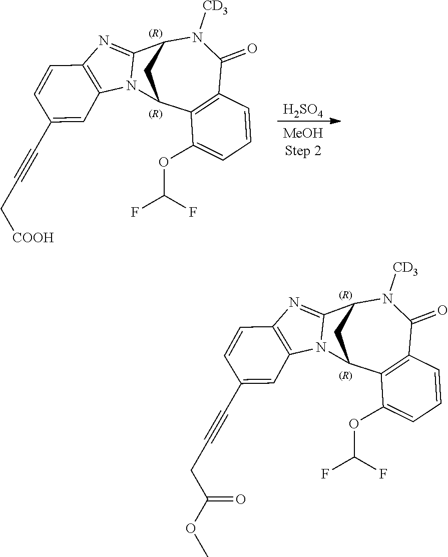

To a stirred solution of 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-ynoic acid (65 mg, 0.148 mmol) in MeOH (2 mL) was added H₂SO₄ (200 uL, 3.752 mmol) dropwise at room temperature. The resulting mixture was stirred for 2 h at 80° C. under nitrogen atmosphere. The mixture was allowed to cool down to room temperature. The reaction was quenched with water at room temperature. The resulting mixture was extracted with CH₂Cl₂ (3×4 mL). The combined organic layers were washed with brine (3×6 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 35% to 55% gradient in 20 min to afford methyl 4-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)but-3-ynoate (23 mg, 33%) as an orange solid. MS ESI calculated for C₂₄H₁₆D₃F₂N₃O₄ [M+H]⁺, 455.15. found 455.20. ¹H NMR (400 MHz, DMSO-d₆) δ 8.31-8.24 (m, 1H), 7.65-7.18 (m, 6H), 6.27-6.19 (m, 1H), 5.25-5.18 (m, 1H), 3.72 (s, 2H), 3.68 (s, 3H), 3.55-3.46 (m, 1H), 2.84-2.75 (m, 1H). ¹⁹F NMR (377 MHz, DMSO-d₆) δ −81.93.

### Example 139 and 140: (7R,14R)-1-(difluoromethoxy)-11-((R or S)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S or R)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one

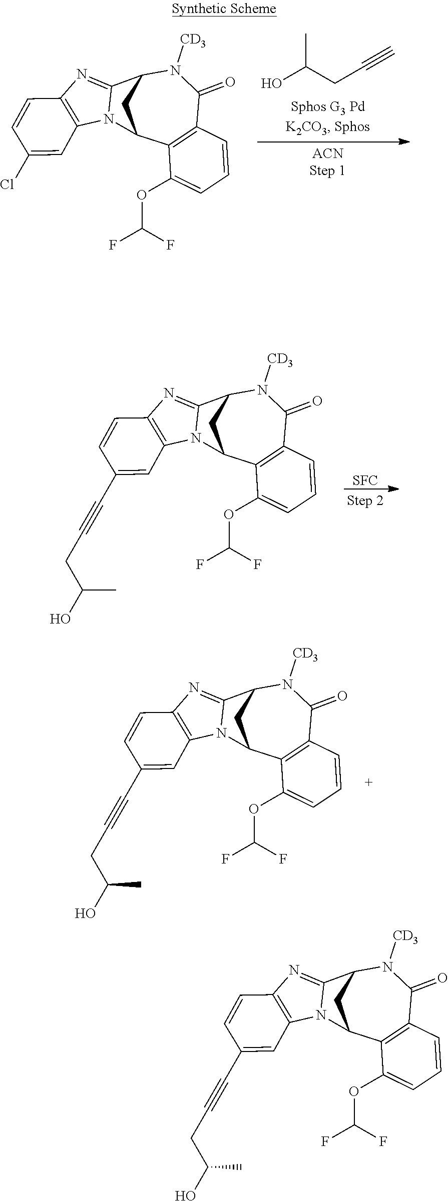

### Preparation 139A: (7R,14R)-1-(difluoromethoxy)-11-(4-hydroxypent-1-yn-1-yl)-6-(methyl-d3)-6,7-dihydro-7,14-methanobenzo[f]benzo[4,5]imidazo[1,2-a][1,4]diazocin-5(14H)-one

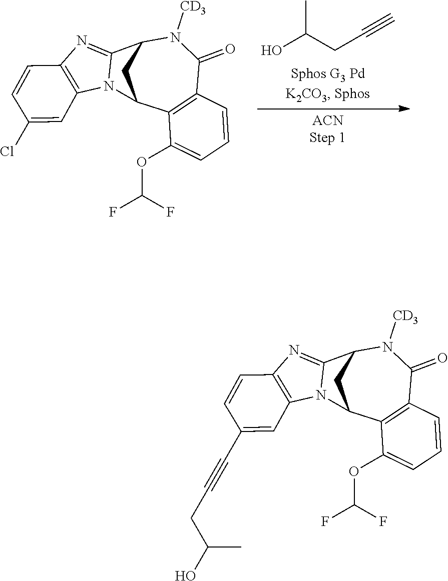

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (200 mg, 0.509 mmol) and pent-4-yn-2-ol (214 mg, 2.545 mmol) in ACN (5 mL) were added K₂CO₃ (352 mg, 2.545 mmol), SPhos (42 mg, 0.102 mmol) and SPhos Pd Gen.3 (80 mg, 0.102 mmol) at room temperature under nitrogen atmosphere. The resulting mixture was stirred for 2 h at 80° C. under nitrogen atmosphere. The resulting mixture was concentrated under reduced pressure and purified by silica gel column chromatography, eluted with CH₂Cl₂/MeOH (10/1) to afford (7R,14R)-1-(difluoromethoxy)-11-(4-hydroxypent-1-yn-1-yl)-6-(methyl-d3)-6,7-dihydro-7,14-methanobenzo[f]benzo[4,5]imidazo[1,2-a][1,4]diazocin-5(14H)-one (130 mg, 58%) as a light yellow solid. MS ESI calculated for C₂₄H₁₈D₃F₂N₃O₃[M+H]⁺, 441.17. found 441.30.

### Example 139 and 140: (7R,14R)-1-(difluoromethoxy)-11-((R or S)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S or R)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one

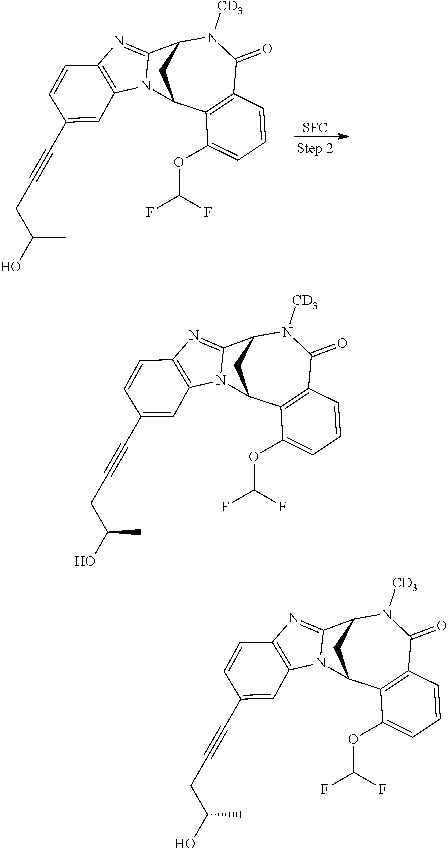

The crude product (180 mg) was resolved by SFC with the following conditions (Column: CHIRALPAK AS-H, 3*25 cm, 5 μm; Mobile Phase A: CO₂, Mobile Phase B: MeOH:EtOH:Hex=1:1:3(20 mM NH₃); Flow rate: 100 mL/min; Gradient: isocratic 25% B; Column Temperature (° C.): 35; Back Pressure (bar): 100; Wave Length: 242/272 nm; RT1(min): 13.15; RT2(min): 14.67. The first peak afforded (50 mg, 28%) as a white solid. MS ESI calculated for C₂₄H₁₈D₃F₂N₃O₃[M+H]⁺, 441.17. found 441.05. ¹H NMR (400 MHz, DMSO-d₆) δ 8.30-8.25 (m, 1H), 7.77-7.38 (m, 5H), 7.22-7.16 (m, 1H), 6.21 (d, J=7.1 Hz, 1H), 5.21 (d, J=7.1 Hz, 1H), 4.83 (d, J=4.6 Hz, 1H), 3.89-3.78 (m, 1H), 3.54-3.44 (m, 1H), 2.80 (d, J=13.7 Hz, 1H), 2.58-2.52 (m, 1H), 2.47-2.37 (m, 1H), 1.20 (d, J=6.1 Hz, 3H). ¹⁹F NMR (377 MHz, DMSO-d₆) δ −82.09.

The second peak afforded (50 mg, 28%) as a white solid. MS ESI calculated for C₂₄H₁₈D₃F₂N₃O₃[M+H]⁺, 441.17. found 441.05. ¹H NMR (400 MHz, DMSO-d₆) δ 8.34-8.23 (m, 1H), 7.77-7.39 (m, 5H), 7.20 (d, J=8.4 Hz, 1H), 6.21 (d, J=7.1 Hz, 1H), 5.21 (d, J=7.1 Hz, 1H), 4.90-4.81 (m, 1H), 3.90-3.77 (m, 1H), 3.56-3.43 (m, 1H), 2.80 (d, J=13.8 Hz, 1H), 2.59-2.52 (m, 1H), 2.47-2.37 (m, 1H), 1.20 (d, J=6.1 Hz, 3H). ¹⁹F NMR (377 MHz, DMSO-d₆) δ −82.10.

### Example 141: 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutane-1-carbonitrile

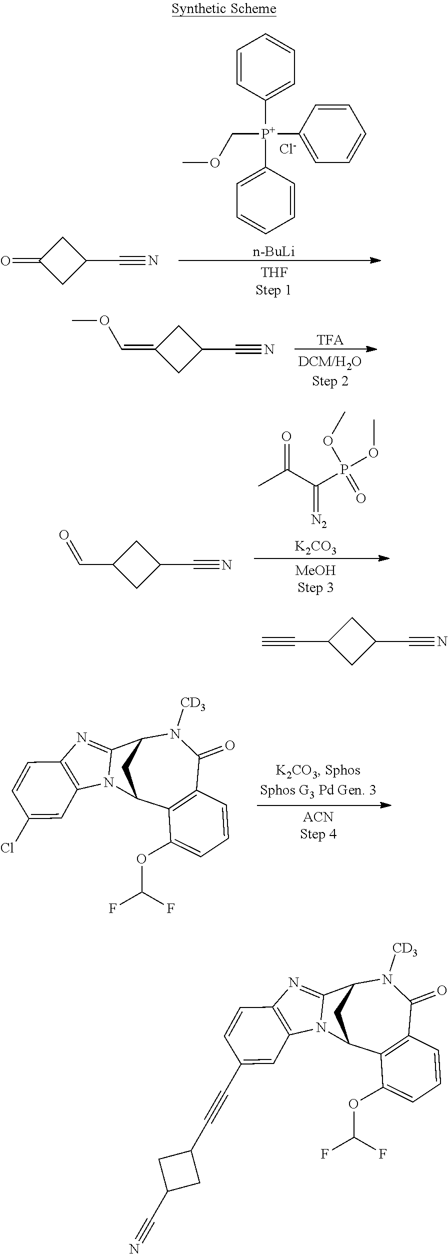

Preparation 141A: 3-(methoxymethylidene)cyclobutane-1-carbonitrile

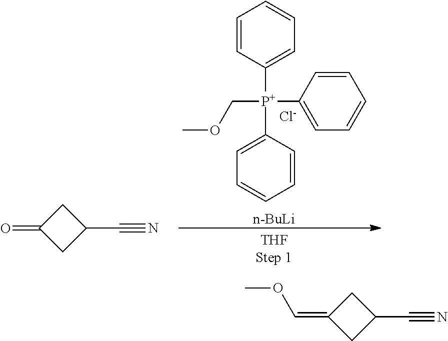

To a stirred mixture of (Methoxymethyl)triphenylphosphonium chloride (5.06 g, 14.721 mmol) in THF (84 mL) was added n-BuLi (6.7 mL, 16.782 mmol) at 0° C. under nitrogen atmosphere. The resulting mixture was stirred for 40 min at 0° C. under nitrogen atmosphere. To the above mixture was added 3-oxocyclobutane-1-carbonitrile (1.40 g, 14.721 mmol) at 0° C. The resulting mixture was stirred for additional 16 h at room temperature. The reaction was quenched with water at room temperature. The aqueous layer was extracted with CH₂Cl₂ (3×100 mL). The combined organic layer was concentrated under vacuum. The residue was purified by silica gel column chromatography, eluted with PE/EA (2:1) to afford 3-(methoxymethylidene)cyclobutane-1-carbonitrile (390 mg, 21%) as a yellow oil. MS ESI calculated for C₇H₉NO [M+H]⁺, 124.07 found N/A. ¹H NMR (300 MHz, Chloroform-d) δ 5.89-5.85 (m, 1H), 3.58 (s, 3H), 3.20-3.15 (m, 1H), 3.15-3.09 (m, 2H), 3.08-3.03 (m, 2H).

### Preparation 141B: 3-formylcyclobutane-1-carbonitrile

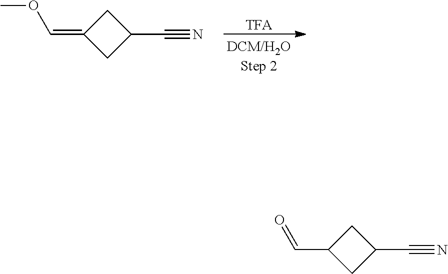

To a stirred solution of 3-(methoxymethylidene)cyclobutane-1-carbonitrile (290 mg, 2.355 mmol) in H₂O (0.3 mL) and DCM (5.8 mL) was added TFA (2 mL) at 0° C. The resulting mixture was stirred for 1 h at room temperature. The resulting mixture was concentrated under vacuum. This resulted in 3-formylcyclobutane-1-carbonitrile (290 mg) as a yellow oil. MS ESI calculated for C₆H₇NO [M+H]⁺, 110.05 found N/A. ¹H NMR (400 MHz, Chloroform-d) δ 9.81-9.64 (m, 1H), 3.28-3.07 (m, 2H), 2.70-2.55 (m, 4H).

### Preparation 141C: 3-ethynylcyclobutane-1-carbonitrile

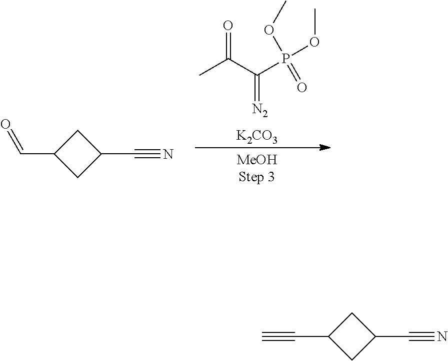

To a stirred mixture of 3-formylcyclobutane-1-carbonitrile (200 mg, 1.833 mmol) and K₂CO₃ (506 mg, 3.666 mmol) in MeOH (2 mL) was added seyferth-gilbert homologation (528 mg, 2.749 mmol) at 0° C. The resulting mixture was stirred for 3 h at room temperature under nitrogen atmosphere. The resulting mixture was diluted with DCM (5 mL). The resulting mixture was filtered, the filter cake was washed with DCM (3×10 mL). The filtrate was concentrated under reduced pressure. The residue was purified by silica gel column chromatography, eluted with PE/EA (10:1) to afford 3-ethynylcyclobutane-1-carbonitrile (100 mg, 52%) as a colorless oil. MS ESI calculated for C₇H₇N [M+H]⁺, 106.06 found N/A. ¹H NMR (400 MHz, Chloroform-d) δ 3.10-2.92 (m, 2H), 2.75-2.64 (m, 2H), 2.59-2.47 (m, 2H), 2.24 (s, 1H).

### Example 141: 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutane-1-carbonitrile

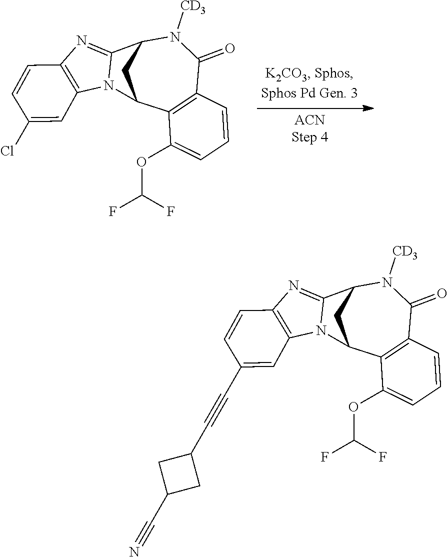 [SMILES: [2H]C([2H])([2H])N1C(=O)c2cccc(OC(F)F)c2[C@H]2C[C@@H]1c1nc3ccc(Cl)cc3n12 ; [2H]C([2H])([2H])N1C(=O)c2cccc(OC(F)F)c2[C@H]2C[C@@H]1c1nc3ccc(C#CC4CC(C#N)C4)cc3n12]

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) and 3-ethynylcyclobutane-1-carbonitrile (94 mg, 0.890 mmol) in ACN (2 mL) were added K₂CO₃ (123 mg, 0.890 mmol), SPhos (15 mg, 0.036 mmol) and SPhos Pd Gen.3 (28 mg, 0.036 mmol) at room temperature under nitrogen atmosphere. The resulting mixture was stirred for 4 h at 80° C. under nitrogen atmosphere. The resulting mixture was concentrated under vacuum. The residue was purified by silica gel column chromatography, eluted with CH₂Cl₂/MeOH (20:1) followed by reversed-phase flash chromatography with the following conditions: column, C18 silica gel; mobile phase, CH₃CN in Water (10 mmol/L NH₄HCO₃), 20% to 50% gradient in 25 min; detector, 254 nm. This resulted in 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutane-1-carbonitrile (7 mg, 8%) as a white solid. MS ESI calculated for C₂₆H₁₇D₃F₂N₄O₂ [M+H]⁺, 462.17. found 462.15. ¹H NMR (400 MHz, DMSO-d₆) δ 8.29-8.25 (m, 1H), 7.78-7.40 (m, 5H), 7.24-7.20 (m, 1H), 6.22 (d, J=7.1 Hz, 1H), 5.22 (d, J=7.1 Hz, 1H), 3.50 (d, J=6.9 Hz, 1H), 2.80 (d, J=13.8 Hz, 1H), 3.33-3.28 (m, 2H), 2.77-2.66 (m, 2H), 2.43-2.32 (m, 2H). ¹⁹F NMR (377 MHz, DMSO-d₆) δ −81.63, −82.08, −82.18, −82.63.

### Example 142: (7R,14R)-1-(difluoromethoxy)-11-((S)-4-methoxy-3-methylbut-1-yn-1-yl)-6-(methyl-d3)-6,7-dihydro-7,14-methanobenzo[f]benzo[4,5]imidazo[1,2-a][1,4]diazocin-5(14H)-one

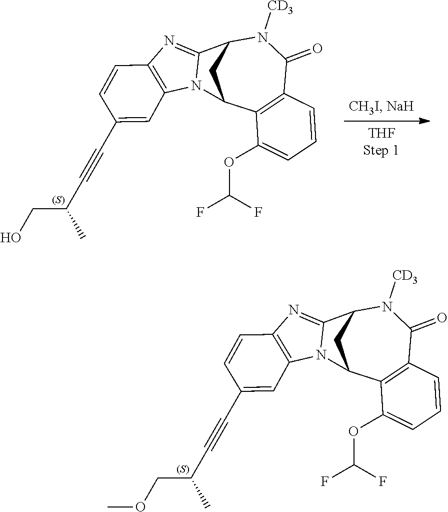

To a stirred solution of (7R,14R)-1-(difluoromethoxy)-11-((S)-4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (28 mg, 0.064 mmol) in THF (2 mL) was added NaH (13 mg, 0.320 mmol, 60%) in portions at 0° C. under nitrogen atmosphere. The resulting mixture was stirred for 10 min at 50° C. under nitrogen atmosphere. To the above mixture was added CH₃I (45 mg, 0.320 mmol) dropwise at room temperature. The resulting mixture was stirred for additional 2 h at room temperature. The reaction was quenched with Water at room temperature. The resulting mixture was extracted with CH₂Cl₂ (3×10 mL). The combined organic layers were washed with brine (2×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 30% to 50% gradient in 20 min; detector, 254/220 nm to afford (7R,14R)-1-(difluoromethoxy)-11-((S)-4-methoxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (16 mg, 54%) as a white solid. MS ESI calculated for C₂₅H₂₀D₃F₂N₃O₃[M+H]⁺, 455.19. found 455.25. ¹H NMR (400 MHz, Chloroform-d) δ 8.51-8.44 (m, 1H), 7.64-7.57 (m, 1H), 7.53-7.48 (m, 1H), 7.47-7.37 (m, 1H), 7.37-7.26 (m, 2H), 6.84 (t, J=73.3, 72.1 Hz, 1H), 6.20 (d, J=7.2 Hz, 1H), 4.96 (d, J=6.4 Hz, 1H), 3.58-3.50 (m, 1H), 3.42 (s, 3H), 3.50-3.35 (m, 2H), 3.01-2.89 (m, 1H), 2.85 (d, J=13.4 Hz, 1H), 1.29 (d, J=6.9 Hz, 3H). ¹⁹F NMR (377 MHz, Chloroform-d) δ −79.93, −80.37, −81.26, −81.71.

### Example 143: (7R,14R)-1-(difluoromethoxy)-11-(4-(3-hydroxy-3-methylazetidin-1-yl)but-1-yn-1-yl)-6-(methyl-d3)-6,7-dihydro-7,14-methanobenzo[f]benzo[4,5]imidazo[1,2-a][1,4]diazocin-5(14H)-one

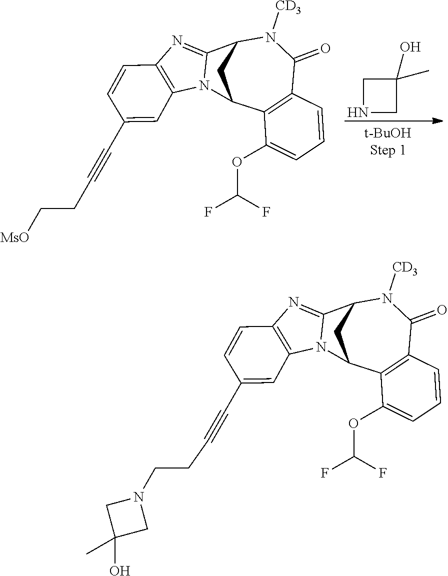

To a stirred solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (30 mg, 0.059 mmol) and 3-methylazetidin-3-ol (52 mg, 0.590 mmol) in t-BuOH (0.5 mL) at room temperature. The resulting mixture was stirred for 6 h at 50° C. The resulting mixture was concentrated under vacuum and purified by reversed-phase flash chromatography with the following conditions: column, C18 silica gel; mobile phase, CH₃CN in Water (10 mmol/L NH₄HCO₃), 30% to 50% gradient in 20 min; detector, 254/220 nm to afford (7R,14R)-1-(difluoromethoxy)-11-(4-(3-hydroxy-3-methylazetidin-1-yl)but-1-yn-1-yl)-6-(methyl-d3)-6,7-dihydro-7,14-methanobenzo[f]benzo[4,5]imidazo[1,2-a][1,4]diazocin-5(14H)-one (14 mg, 47%) as a white solid. MS ESI calculated for C₂₇H₂₃D₃F₂N₄O₃ [M+H]⁺, 496.22. found 496.30. ¹H NMR (400 MHz, Chloroform-d) δ 8.51-8.44 (m, 1H), 7.60 (d, J=8.4 Hz, 1H), 7.49 (d, J=1.6 Hz, 1H), 7.41 (t, J=8.2 Hz, 1H), 7.34 (d, J=8.1 Hz, 1H), 7.24 (d, J=1.6 Hz, 1H), 6.88 (t, J=73.3 Hz, 1H), 6.20 (d, J=7.2 Hz, 1H), 4.92 (d, J=7.1 Hz, 1H), 3.53-3.31 (m, 5H), 2.89-2.80 (m, 3H), 2.57 (t, J=7.0 Hz, 2H), 1.51 (s, 3H). ¹⁹F NMR (377 MHz, Chloroform-d) δ −79.95, −80.39, −81.15, −81.59.

### Example 144: (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one

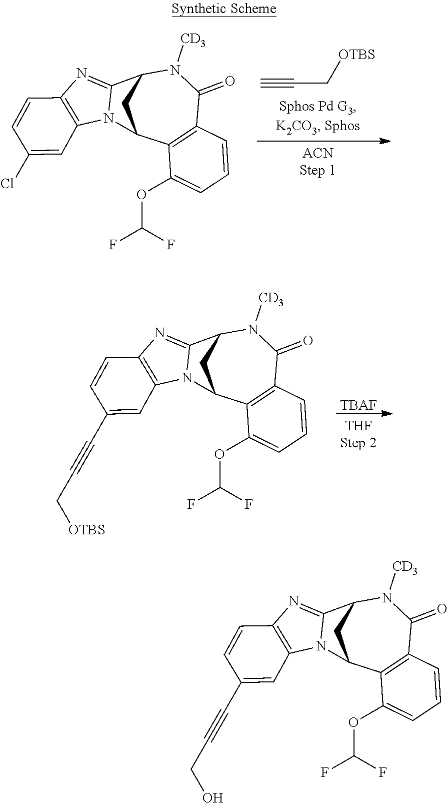

### Preparation 144A: (7R,14R)-11-(3-((tert-butyldimethylsilyl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

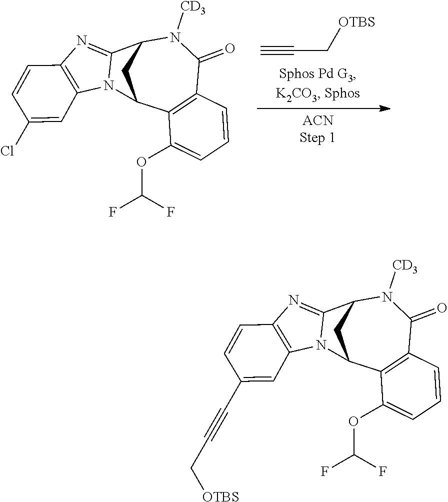

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (500 mg, 1.273 mmol) and tert-butyldimethyl(prop-2-yn-1-yloxy)silane (2.17 g, 12.730 mmol) in ACN (10 mL) were added SPhos (105 mg, 0.255 mmol), SPhos Pd Gen.3 (199 mg, 0.255 mmol) and K₂CO₃ (880 mg, 6.365 mmol) at room temperature under nitrogen atmosphere. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) to afford (7R,14R)-11-(3-((tert-butyldimethylsilyl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (650 mg, 97%) as a brown solid. MS ESI calculated for C₂₈H₂₈D₃F₂N₃O₃Si [M+H]⁺, 527.23. found 527.15.

### Example 144: (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one

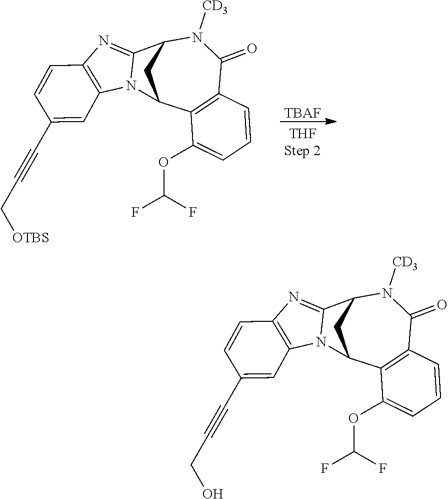

A solution of (7R,14R)-11-(3-((tert-butyldimethylsilyl)oxy)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (650 mg, 1.234 mmol) and TBAF (1.48 mL, 1.481 mmol) in THF (10 mL) was stirred for 1 h at room temperature under nitrogen atmosphere. The resulting mixture was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one (410 mg, 80%) as a white solid. MS ESI calculated for C₂₂H₁₄D₃F₂N₃O₃ [M+H]⁺, 413.14, found 413.15. ¹H NMR (400 MHz, Chloroform-d) δ 8.51-8.44 (m, 1H), 7.62 (d, J=8.5 Hz, 1H), 7.52 (d, J=1.5 Hz, 1H), 7.42 (t, J=8.2 Hz, 1H), 7.34 (d, J=8.1 Hz, 1H), 7.28 (d, J=1.5 Hz, 1H), 6.84 (t, J=73.2 Hz, 1H), 6.22 (d, J=7.2 Hz, 1H), 5.01 (d, J=7.1 Hz, 1H), 4.52 (s, 2H), 3.52-3.40 (m, 1H), 2.86 (d, J=13.6 Hz, 1H). ¹⁹F NMR (377 MHz, Chloroform-d) δ −79.96, −80.41, −81.10, −81.54.

### Example 145: (7R,14R)-1-(difluoromethoxy)-9-fluoro-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one

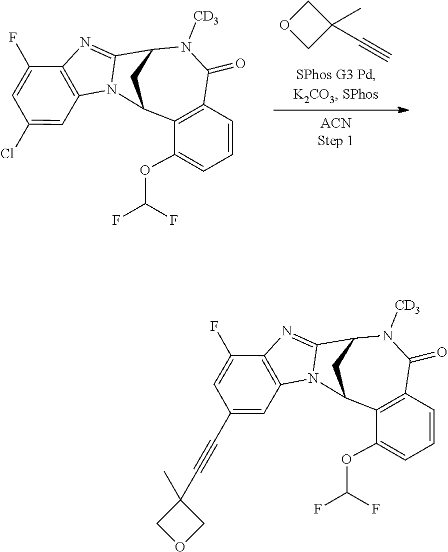

To a solution of (7R,14R)-11-chloro-1-(difluoromethoxy)-9-fluoro-6-(methyl-d3)-6,7-dihydro-7,14-methanobenzo[f]benzo[4,5]imidazo[1,2-a][1,4]diazocin-5(14H)-one (60 mg, 0.146 mmol) and 3-ethynyl-3-methyloxetane (70 mg, 0.730 mmol) in ACN (1 mL) were added K₂CO₃ (101 mg, 0.730 mmol) and SPhos (12 mg, 0.029 mmol), SPhos Pd Gen.3 (23 mg, 0.029 mmol). After stirring for overnight at 80° C. under a nitrogen atmosphere, the resulting mixture was concentrated under reduced pressure. The residue was purified by silica gel column chromatography, eluted with PE/EA (0~100%) followed purified by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: ACN; Flow rate: 60 mL/min mL/min; Gradient: 31% B to 46% B in 10 min; Wave Length: 254 nm/220 nm; RT1(min): 11.55). This resulted in (7R,14R)-1-(difluoromethoxy)-9-fluoro-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one (21 mg, 30%) as a white solid. MS ESI calculated for C₂₅H₁₇D₃F₃N₃O₃ [M+H]⁺, 471.16. found 471.05. ¹H NMR (400 MHz, Chloroform-d) δ 8.53-8.47 (m, 1H), 7.44 (t, J=8.2 Hz, 1H), 7.36-7.33 (m, 1H), 7.31 (d, J=1.2 Hz, 1H), 7.02 (d, J=1.3 Hz, 1H), 7.00-6.64 (m, 1H), 6.21 (d, J=7.2 Hz, 1H), 5.00-4.87 (m, 3H), 4.48 (d, J=5.5 Hz, 2H), 3.52-3.41 (m, 1H), 2.87 (d, J=13.6 Hz, 1H), 1.72 (s, 3H). ¹⁹F NMR (377 MHz, Chloroform-d) δ −80.08, −80.53, −81.18, −81.63, −126.77.

### Example 146: (7R,14R)-1-(difluoromethoxy)-12-fluoro-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one

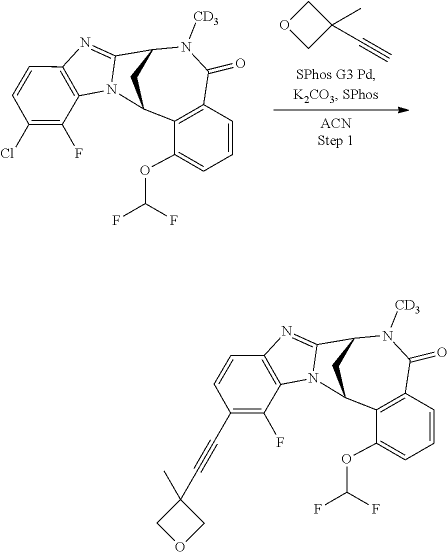

To a solution of (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (60 mg, 0.146 mmol) and 3-ethynyl-3-methyloxetane (70 mg, 0.730 mmol) in ACN (1 mL) were added K₂CO₃ (101 mg, 0.730 mmol) and SPhos (12 mg, 0.029 mmol), SPhos Pd Gen.3 (23 mg, 0.029 mmol). After stirring for overnight at 80° C. under a nitrogen atmosphere, the resulting mixture was concentrated under reduced pressure. The residue was purified by silica gel column chromatography, eluted with PE/EA (0~100%) followed purified by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: ACN; Flow rate: 60 mL/min mL/min; Gradient: 34% B to 48% B in 10 min; Wave Length: 254 nm; RT1(min): 9.9). This resulted in (7R,14R)-1-(difluoromethoxy)-12-fluoro-6-(methyl-d3)-11-((3-methyloxetan-3-yl)ethynyl)-6,7-dihydro-7,14-methanobenzo[f]benzo[4,5]imidazo[1,2-a][1,4]diazocin-5(14H)-one (15 mg, 22%) as a white solid. MS ESI calculated for C₂₅H₁₇D₃F₃N₃O₃ [M+H]⁺, 471.16. found 471.05. ¹H NMR (400 MHz, Chloroform-d) δ 8.47-8.40 (m, 1H), 7.48-7.40 (m, 2H), 7.40-7.34 (m, 1H), 7.27-7.19 (m, 1H), 6.97-6.56 (m, 1H), 6.49 (d, J=7.2 Hz, 1H), 4.99 (d, J=6.8 Hz, 1H), 4.93 (d, J=5.4 Hz, 2H), 4.48 (d, J=5.5 Hz, 2H), 3.53-3.41 (m, 1H), 2.86 (d, J=13.5 Hz, 1H), 1.73 (s, 3H). ¹⁹F NMR (377 MHz, Chloroform-d) δ −78.58, −79.02, −82.59, −82.62, −83.04, −83.06, −129.22.

### Example 147: (7R,14R)-1-(difluoromethoxy)-11-((R)-4-methoxy-3-methylbut-1-yn-1-yl)-6-(methyl-d3)-6,7-dihydro-7,14-methanobenzo[f]benzo[4,5]imidazo[1,2-a][1,4]diazocin-5(14H)-one

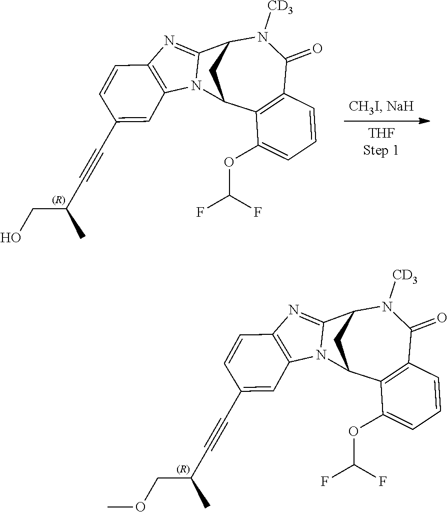

To a stirred solution of (7R,14R)-1-(difluoromethoxy)-11-((R)-4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (40 mg, 0.091 mmol) in THF (2 mL) was added NaH (18 mg, 0.455 mmol, 60%) in portions at 0° C. under nitrogen atmosphere. The resulting mixture was stirred for 10 min at 50° C. under nitrogen atmosphere. To the above mixture was added CH₃I (64 mg, 0.455 mmol) dropwise at room temperature. The resulting mixture was stirred for additional 2 h at room temperature. The reaction was quenched with Water at room temperature. The resulting mixture was extracted with CH₂Cl₂ (3×10 mL). The combined organic layers were washed with brine (2×10 mL), dried over anhydrous Na₂SO₄. After filtration, the filtrate was concentrated under reduced pressure. The crude product was purified by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: ACN; Flow rate: 60 mL/min; Gradient: 33% B to 51% B in 10 min; Wave Length: 254/220 nm) to afford (7R,14R)-1-(difluoromethoxy)-11-((R)-4-methoxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (12 mg, 30%) as a white solid. MS ESI calculated for C₂₅H₂₀D₃F₂N₃O₃[M+H]⁺, 455.19. found 455.25. ¹H NMR (400 MHz, Chloroform-d) δ 8.52-8.45 (m, 1H), 7.62 (d, J=8.5 Hz, 1H), 7.55-7.51 (m, 1H), 7.46-7.41 (m, 1H), 7.37-7.30 (m, 2H), 6.84 (t, J=73.2 Hz, 1H), 6.23 (d, J=7.1 Hz, 1H), 5.03 (d, J=7.0 Hz, 1H), 3.56-3.51 (m, 1H), 3.51-3.44 (m, 1H), 3.42 (s, 3H), 3.41-3.38 (m, 1H), 3.00-2.90 (m, 1H), 2.87 (d, J=13.6 Hz, 1H), 1.29 (d, J=6.9 Hz, 3H). ¹⁹F NMR (377 MHz, Chloroform-d) δ −79.98, −80.43, −81.24, −81.68.

### Example 148: (7R,14R)-1-(difluoromethoxy)-11-((R or S)-4-methoxypent-1-yn-1-yl)-6-(methyl-d3)-6,7-dihydro-7,14-methanobenzo[f]benzo[4,5]imidazo[1,2-a][1,4]diazocin-5(14H)-one

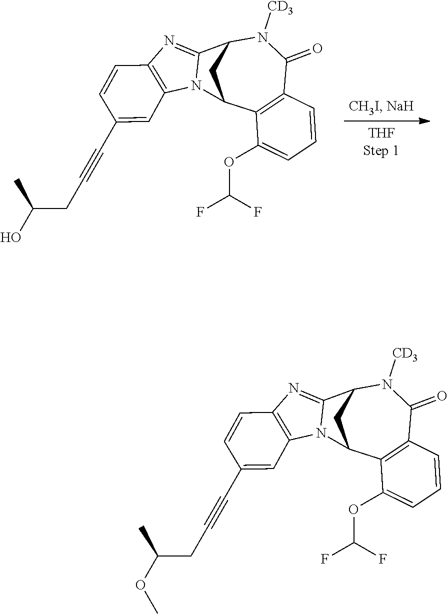 [SMILES: [2H]C([2H])([2H])N1C(=O)c2cccc(OC(F)F)c2[C@H]2C[C@@H]1c1nc3ccc(C#CC[C@H](C)O)cc3n12 ; [2H]C([2H])([2H])N1C(=O)c2cccc(OC(F)F)c2[C@H]2C[C@@H]1c1nc3ccc(C#CC[C@H](C)OC)cc3n12]

To a stirred solution of (7R,14R)-1-(difluoromethoxy)-11-((R or S)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one (40 mg, 0.091 mmol) in THF (2 mL) was added NaH (18 mg, 0.455 mmol, 60%) in portions at 0° C. under nitrogen atmosphere. The resulting mixture was stirred for 10 min at 50° C. under nitrogen atmosphere. To the above mixture was added CH₃I (64 mg, 0.455 mmol) dropwise at room temperature. The resulting mixture was stirred for additional 2 h at room temperature. The reaction was quenched with water at 0° C. The resulting mixture was extracted with CH₂Cl₂ (3×10 mL). The combined organic layers were washed with brine (2×10 mL), dried over anhydrous Na₂SO₄. After filtration, the filtrate was concentrated under reduced pressure. The crude product was purified by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min; Gradient: 32% B to 52% B in 12 min; Wave Length: 220 nm) to afford (7R,14R)-1-(difluoromethoxy)-11-((R or S)-4-methoxypent-1-yn-1-yl)-6-(methyl-d3)-6,7-dihydro-7,14-methanobenzo[f]benzo[4,5]imidazo[1,2-a][1,4]diazocin-5(14H)-one (22 mg, 53%) as a white solid. MS ESI calculated for C₂₅H₂₀D₃F₂N₃O₃[M+H]⁺, 455.19. found 455.25. ¹H NMR (400 MHz, Chloroform-d) δ 8.53-8.44 (m, 1H), 7.64 (d, J=8.5 Hz, 1H), 7.56-7.51 (m, 1H), 7.48-7.40 (m, 1H), 7.37-7.31 (m, 2H), 6.84 (t, J=73.1 Hz, 1H), 6.25 (d, J=7.2 Hz, 1H), 5.06 (d, J=7.1 Hz, 1H), 3.63-3.53 (m, 1H), 3.52-3.43 (m, 1H), 3.41 (s, 3H), 2.88 (d, J=13.6 Hz, 1H), 2.75-2.65 (m, 1H), 2.58-2.48 (m, 1H), 1.33 (d, J=6.1 Hz, 3H). ¹⁹F NMR (377 MHz, Chloroform-d) δ −80.05, −80.50, −81.13, −81.57.

### Example 149: (7R,14R)-1-(difluoromethoxy)-11-((S or R)-4-methoxypent-1-yn-1-yl)-6-(methyl-d3)-6,7-dihydro-7,14-methanobenzo[f]benzo[4,5]imidazo[1,2-a][1,4]diazocin-5(14H)-one

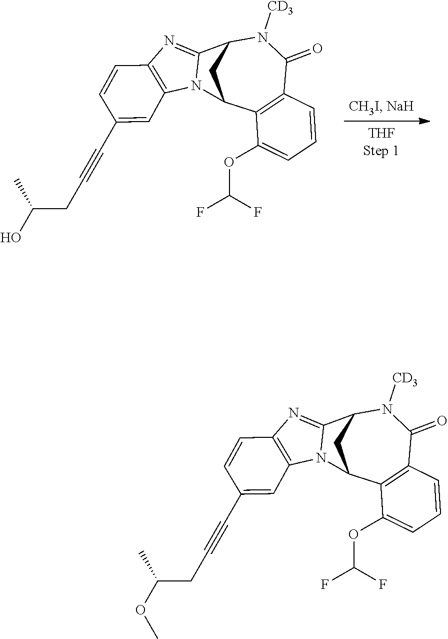 [SMILES: [2H]C([2H])([2H])N1C(=O)c2cccc(OC(F)F)c2[C@H]2C[C@@H]1c1nc3ccc(C#CC[C@@H](C)O)cc3n12 ; [2H]C([2H])([2H])N1C(=O)c2cccc(OC(F)F)c2[C@H]2C[C@@H]1c1nc3ccc(C#CC[C@@H](C)OC)cc3n12]

To a stirred solution of (7R,14R)-1-(difluoromethoxy)-11-((S or R)-4-hydroxypent-1-yn-1-yl)-6-(methyl-d3)-6,7-dihydro-7,14-methanobenzo[f]benzo[4,5]imidazo[1,2-a][1,4]diazocin-5(14H)-one (40 mg, 0.091 mmol) in THF (2 mL) was added NaH (18 mg, 0.455 mmol, 60%) in portions at 0° C. under nitrogen atmosphere. The resulting mixture was stirred for 10 min at 50° C. under nitrogen atmosphere. To the above mixture was added CH₃I (64 mg, 0.455 mmol) dropwise at room temperature. The resulting mixture was stirred for additional 2 h at room temperature. The reaction was quenched with Water at room temperature. The resulting mixture was extracted with CH₂Cl₂ (3×10 mL). The combined organic layers were washed with brine (2×10 mL), dried over anhydrous Na₂SO₄. After filtration, the filtrate was concentrated under reduced pressure. The crude product was purified by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min; Gradient: 33% B to 53% B in 10 min; Wave Length: 254/220 nm) to afford (7R,14R)-1-(difluoromethoxy)-11-((S or R)-4-methoxypent-1-yn-1-yl)-6-(methyl-d3)-6,7-dihydro-7,14-methanobenzo[f]benzo[4,5]imidazo[1,2-a][1,4]diazocin-5(14H)-one (20 mg, 48%) as a white solid. MS ESI calculated for C₂₅H₂₀D₃F₂N₃O₃[M+H]⁺, 455.19. found 455.20. ¹H NMR (400 MHz, Chloroform-d) δ 8.51-8.46 (m, 1H), 7.66-7.61 (m, 1H), 7.56-7.52 (m, 1H), 7.47-7.41 (m, 1H), 7.36-7.30 (m, 2H), 6.84 (t, J=73.1 Hz, 1H), 6.24 (d, J=7.1 Hz, 1H), 5.04 (d, J=6.9 Hz, 1H), 3.63-3.53 (m, 1H), 3.52-3.43 (m, 1H), 3.41 (s, 3H), 2.88 (d, J=13.6 Hz, 1H), 2.75-2.67 (m, 1H), 2.58-2.49 (m, 1H), 1.33 (d, J=6.1 Hz, 3H). ¹⁹F NMR (377 MHz, Chloroform-d) δ −80.05, −80.50, −81.11, −81.56.

### Example 150: (7R,14R)-11-((3,3-difluorocyclobutyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

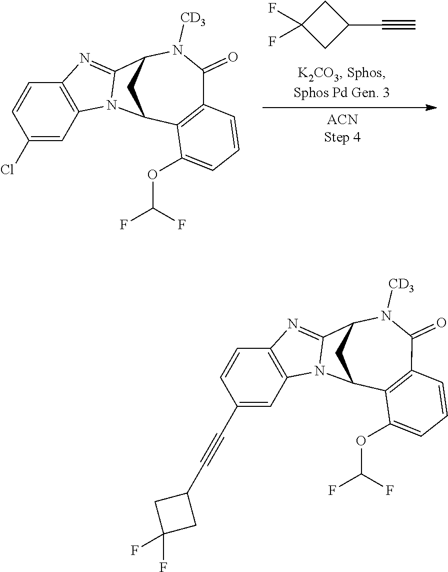

To a stirred mixture of 3-ethynyl-1,1-difluorocyclobutane (62 mg, 0.534 mmol) and (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol) in ACN (2 mL) were added K₂CO₃ (123 mg, 0.890 mmol), SPhos (15 mg, 0.036 mmol) and SPhos Pd Gen.3 (28 mg, 0.036 mmol) at room temperature under nitrogen atmosphere. The resulting mixture was stirred for 3 h at 80° C. under nitrogen atmosphere. The resulting mixture was concentrated under vacuum and purified by silica gel column chromatography, eluted with CH₂Cl₂/MeOH (20:1) followed by reversed-phase flash chromatography with the following conditions: column, C18 silica gel; mobile phase, CH₃CN in Water (10 mmol/L NH₄HCO₃), 30% to 60% gradient in 25 min; detector, 254 nm. This resulted in (7R,14R)-11-((3,3-difluorocyclobutyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (31 mg, 36%) as a white solid. MS ESI calculated for C₂₅H₁₆D₃F₄N₃O₂ [M+H]⁺, 473.16. found 473.15. ¹H NMR (400 MHz, Chloroform-d) δ 8.51-8.45 (m, 1H), 7.62 (d, J=8.4 Hz, 1H), 7.50 (d, J=1.6 Hz, 1H), 7.45-7.38 (m, 1H), 7.33 (d, J=8.1 Hz, 1H), 7.29-7.25 (m, 1H), 7.04-6.65 (m, 1H), 6.20 (d, J=7.3 Hz, 1H), 4.93 (d, J=7.1 Hz, 1H), 3.49-3.36 (m, 1H), 3.19-3.07 (m, 1H), 3.04-2.92 (m, 2H), 2.89-2.82 (m, 1H), 2.82-2.69 (m, 2H). ¹⁹F NMR (377 MHz, Chloroform-d) δ −79.95, −80.39, −81.09, −81.54, −82.64, −83.16, −96.83, −97.35.

### Example 151: (7R,14R)-1-(difluoromethoxy)-6-(methyl-d3)-11-(4-(methylamino)but-1-yn-1-yl)-6,7-dihydro-7,14-methanobenzo[f]benzo[4,5]imidazo[1,2-a][1,4]diazocin-5(14H)-one

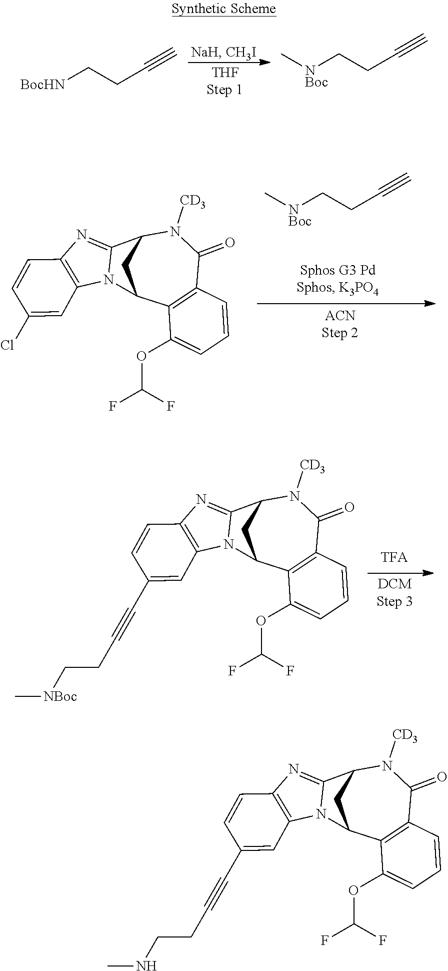 [SMILES: C#CCCNC(=O)OC(C)(C)C ; C#CCCN(C)C(=O)OC(C)(C)C ; [2H]C([2H])([2H])N1C(=O)c2cccc(OC(F)F)c2[C@H]2C[C@@H]1c1nc3ccc(Cl)cc3n12 ; C#CCCN(C)C(=O)OC(C)(C)C ; [2H]C([2H])([2H])N1C(=O)c2cccc(OC(F)F)c2[C@H]2C[C@@H]1c1nc3ccc(C#CCCN(C)C(=O)OC(C)(C)C)cc3n12 ; [2H]C([2H])([2H])N1C(=O)c2cccc(OC(F)F)c2[C@H]2C[C@@H]1c1nc3ccc(C#CCCNC)cc3n12]

### Preparation 151A: tert-butyl N-(but-3-yn-1-yl)-N-methylcarbamate

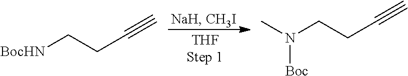

To a solution of tert-butyl N-(but-3-yn-1-yl)carbamate (500 mg, 2.955 mmol) in THF (5 mL) was added NaH (177 mg, 4.433 mmol, 60%) in portions at 0° C. The mixture was stirred for 20 min at 0° C. CH₃I (629 mg, 4.433 mmol) was added and the mixture was allowed to warm to room temperature and stirred for overnight. The reaction mixture was quenched by water and extracted with DCM (3×100 mL), dried over anhydrous Na₂SO₄. After filtration, the filtrate was concentrated under reduced pressure. The residue was purified by silica gel column chromatography, eluted with PE/EA (0~30%) to afford tert-butyl N-(but-3-yn-1-yl)-N-methylcarbamate (370 mg, 49%) as a light yellow liquid. MS ESI calculated for C₁₀H₁₇NO₂ [M+H]⁺, 184.13. found 184.14. ¹H NMR (400 MHz, Chloroform-d) δ 3.34 (t, J=7.0 Hz, 2H), 2.88 (s, 3H), 2.41-2.33 (m, 2H), 1.93 (t, J=2.7 Hz, 1H), 1.43 (s, 9H).

### Preparation 151B: tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(methyl)carbamate

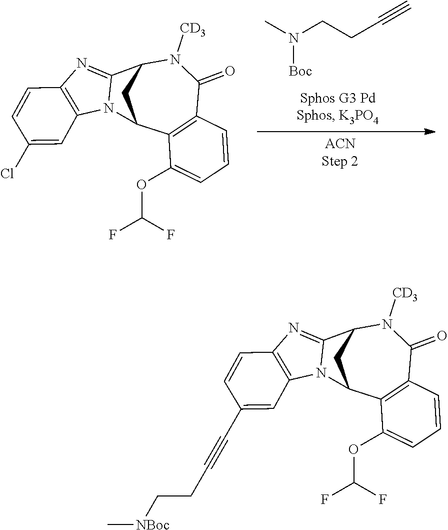 [SMILES: [2H]C([2H])([2H])N1C(=O)c2cccc(OC(F)F)c2[C@H]2C[C@@H]1c1nc3ccc(Cl)cc3n12 ; C#CCCN(C)C(=O)OC(C)(C)C ; [2H]C([2H])([2H])N1C(=O)c2cccc(OC(F)F)c2[C@H]2C[C@@H]1c1nc3ccc(C#CCCN(C)C(=O)OC(C)(C)C)cc3n12]

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and tert-butyl N-(but-3-yn-1-yl)-N-methylcarbamate (186 mg, 1.020 mmol) in ACN (1 mL) were added K₂CO₃ (141 mg, 1.020 mmol) and SPhos (17 mg, 0.041 mmol), SPhos Pd Gen.3 (32 mg, 0.041 mmol). After stirring for 16 h at 80° C. under a nitrogen atmosphere, the resulting mixture was concentrated under reduced pressure. The residue was purified by silica gel column chromatography, eluted with PE/EA (0~100%) followed purified by reversed-phase flash chromatography with the following conditions: column, C18 silica gel; mobile phase, CH₃CN in Water (10 mmol/L NH₄HCO₃), 50% to 70% gradient in 20 min; detector, 254 nm. This resulted in tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(methyl)carbamate (84 mg, 76%) as a white solid. MS ESI calculated for C₂₉H₂₇D₃F₂N₄O₄[M+H]⁺, 540.24. found 540.25. ¹H NMR (400 MHz, Chloroform-d) δ 8.49 (d, J=8.2 Hz, 1H), 7.70-7.64 (m, 1H), 7.53 (s, 1H), 7.49-7.42 (m, 1H), 7.39-7.31 (m, 2H), 7.15-6.68 (m, 1H), 6.28 (d, J=7.4 Hz, 1H), 5.14 (d, J=7.0 Hz, 1H), 3.58-3.39 (m, 3H), 2.97 (s, 3H), 2.91 (d, J=13.6 Hz, 1H), 2.68-2.61 (m, 2H), 1.42 (s, 9H).

### Example 151: (7R,14R)-1-(difluoromethoxy)-6-(methyl-d3)-11-(4-(methylamino)but-1-yn-1-yl)-6,7-dihydro-7,14-methanobenzo[f]benzo[4,5]imidazo[1,2-a][1,4]diazocin-5(14H)-one

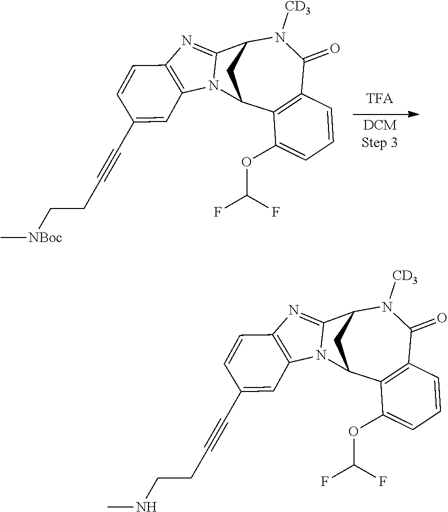 [SMILES: [2H]C([2H])([2H])N1C(=O)c2cccc(OC(F)F)c2[C@H]2C[C@@H]1c1nc3ccc(C#CCCN(C)C(=O)OC(C)(C)C)cc3n12 ; [2H]C([2H])([2H])N1C(=O)c2cccc(OC(F)F)c2[C@H]2C[C@@H]1c1nc3ccc(C#CCCNC)cc3n12]

To a stirred solution of tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(methyl)carbamate (84 mg, 0.156 mmol) in DCM (2 mL) was added TFA (0.4 mL) dropwise at room temperature under nitrogen atmosphere. The mixture was stirred for 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 25% to 50% gradient in 20 min; detector, 254 nm. This resulted in (7R,14R)-1-(difluoromethoxy)-6-(methyl-d3)-11-(4-(methylamino)but-1-yn-1-yl)-6,7-dihydro-7,14-methanobenzo[f]benzo[4,5]imidazo[1,2-a][1,4]diazocin-5(14H)-one (41 mg, 59%) as a white solid. MS ESI calculated for C₂₄H₁₉D₃F₂N₄O₂ [M+H]⁺, 440.19. found 440.10. ¹H NMR (400 MHz, Methanol-d₄) δ 8.45-8.31 (m, 1H), 7.62-7.41 (m, 4H), 7.32-7.05 (m, 2H), 6.34 (d, J=7.2 Hz, 1H), 5.17 (d, J=7.2 Hz, 1H), 3.60-3.48 (m, 1H), 2.87 (d, J=13.9 Hz, 1H), 2.81 (t, J=7.0 Hz, 2H), 2.64 (t, J=6.8 Hz, 2H), 2.45 (s, 3H). ¹⁹F NMR (377 MHz, Methanol-d₄) δ −83.03, −83.48, −83.56, −84.01.

### Example 152: (7R,14R)-1-(difluoromethoxy)-11-(4-(ethylamino)but-1-yn-1-yl)-6-(methyl-d3)-6,7-dihydro-7,14-methanobenzo[f]benzo[4,5]imidazo[1,2-a][1,4]diazocin-5(14H)-one

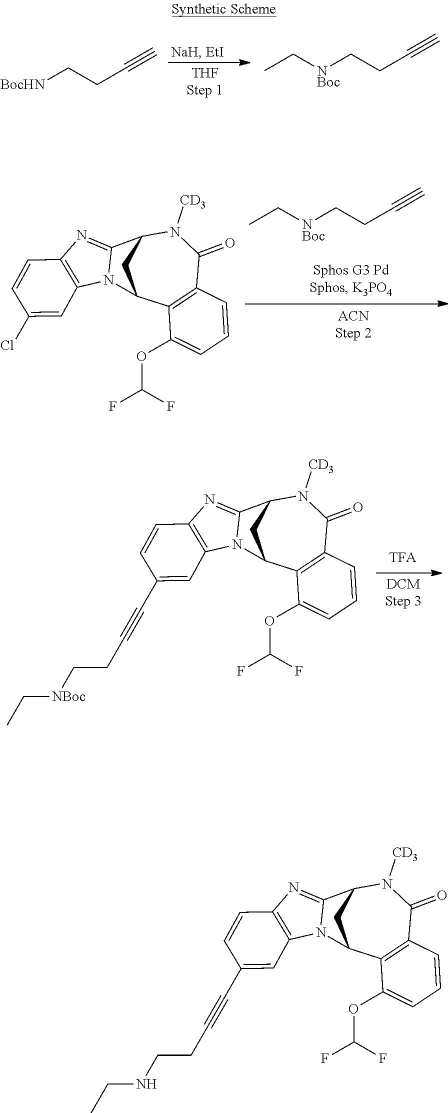

### Preparation 152A: tert-butyl N-(but-3-yn-1-yl)-N-ethylcarbamate

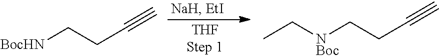

To a solution of tert-butyl N-(but-3-yn-1-yl)carbamate (500 mg, 2.955 mmol) in THF (5 mL) was added NaH (177 mg, 4.433 mmol, 60%) at 0 degrees C. The mixture was stirred for 20 min. Iodoethane (691 mg, 4.433 mmol) was added and the mixture was allowed to warm up to RT and stirred for 16 h. The reaction mixture was quenched by water and extracted with DCM (3×100 mL), dried over anhydrous Na₂SO₄. After filtration, the filtrate was concentrated under reduced pressure. The residue was purified by silica gel column chromatography, eluted with PE/EA (0~40%) to afford tert-butyl N-(but-3-yn-1-yl)-N-ethylcarbamate (475 mg, 82%) as a light yellow liquid. MS ESI calculated for C₁₁H₁₉NO₂ [M+H]⁺, 198.14. found 198.14. ¹H NMR (400 MHz, Chloroform-d) δ 3.36-3.22 (m, 4H), 2.47-2.36 (m, 2H), 1.94 (t, J=2.7 Hz, 1H), 1.44 (s, 9H), 1.14-1.04 (m, 3H).

### Preparation 152B: tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(ethyl)carbamate

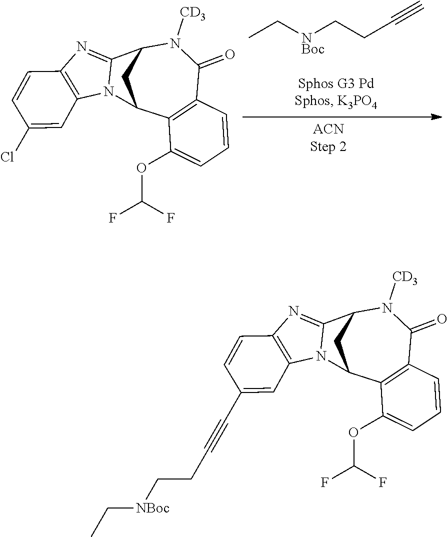

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and tert-butyl N-(but-3-yn-1-yl)-N-ethylcarbamate (200 mg, 1.020 mmol) in ACN (0.5 mL) were added K₂CO₃ (141 mg, 1.020 mmol) and SPhos (17 mg, 0.041 mmol), SPhos Pd Gen.3 (32 mg, 0.041 mmol). After stirring for overnight at 80° C. under a nitrogen atmosphere, the resulting mixture was concentrated under reduced pressure. The residue was purified by silica gel column chromatography, eluted with PE/EA (0~100%) followed purified by reversed-phase flash chromatography with the following conditions: column, C18 silica gel; mobile phase, CH₃CN in Water (10 mmol/L NH₄HCO₃), 50% to 70% gradient in 20 min; detector, 254 nm. This resulted in tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(ethyl)carbamate (80 mg, 71%) as a white solid. MS ESI calculated for C₃₀H₂₉D₃F₂N₄O₄ [M+H]⁺, 554.23. found 554.30. ¹H NMR (400 MHz, Chloroform-d) δ 8.55-8.45 (m, 1H), 7.67-7.63 (m, 1H), 7.55-7.51 (m, 1H), 7.48-7.42 (m, 1H), 7.37 (s, 1H), 7.36-7.30 (m, 1H), 7.09-6.67 (m, 1H), 6.26 (d, J=7.0 Hz, 1H), 5.09 (d, J=8.6 Hz, 1H), 3.54-3.27 (m, 5H), 2.94-2.85 (m, 1H), 2.69-2.63 (m, 2H), 1.45 (s, 9H), 1.15 (t, J=7.1 Hz, 3H).

### Example 152: (7R,14R)-1-(difluoromethoxy)-11-(4-(ethylamino)but-1-yn-1-yl)-6-(methyl-d3)-6,7-dihydro-7,14-methanobenzo[f]benzo[4,5]imidazo[1,2-a][1,4]diazocin-5(14H)-one

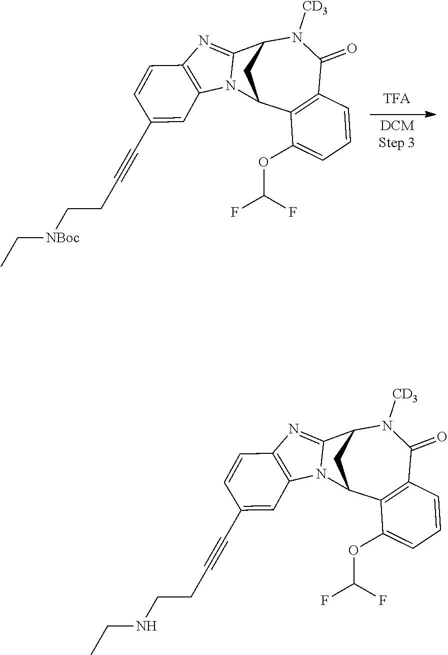 [SMILES: [2H]C([2H])([2H])N1C(=O)c2cccc(OC(F)F)c2[C@H]2C[C@@H]1c1nc3ccc(C#CCCN(CC)C(=O)OC(C)(C)C)cc3n12 ; [2H]C([2H])([2H])N1C(=O)c2cccc(OC(F)F)c2[C@H]2C[C@@H]1c1nc3ccc(C#CCCNCC)cc3n12]

To a stirred solution of tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)(ethyl)carbamate (80 mg, 0.145 mmol) in DCM (2 mL) were added TFA (0.4 mL) dropwise at room temperature under nitrogen atmosphere. The mixture was stirred for 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 25% to 50% gradient in 20 min; detector, 254 nm. This resulted in (7R,14R)-1-(difluoromethoxy)-11-(4-(ethylamino)but-1-yn-1-yl)-6-(methyl-d3)-6,7-dihydro-7,14-methanobenzo[f]benzo[4,5]imidazo[1,2-a][1,4]diazocin-5(14H)-one (13 mg, 20%) as a white solid. ESI calculated for C₂₅H₂₁D₃F₂N₄O₂ [M+H]⁺, 454.21. found 454.20. ¹H NMR (400 MHz, Methanol-d₄) δ 8.39-8.33 (m, 1H), 7.58-7.42 (m, 4H), 7.42-7.05 (m, 2H), 6.34 (d, J=7.2 Hz, 1H), 5.16 (d, J=7.2 Hz, 1H), 3.58-3.47 (m, 1H), 2.89-2.80 (m, 3H), 2.76-2.68 (m, 2H), 2.64 (t, J=6.9 Hz, 2H), 1.15 (t, J=7.2 Hz, 3H). ¹⁹F NMR (377 MHz, Methanol-d₄) δ −82.93, −83.38, −83.50, −83.95.

### Example 153: (7R,14R)-1-(difluoromethoxy)-6-(methyl-d3)-11-(5-(methylamino)pent-1-yn-1-yl)-6,7-dihydro-7,14-methanobenzo[f]benzo[4,5]imidazo[1,2-a][1,4]diazocin-5(14H)-one

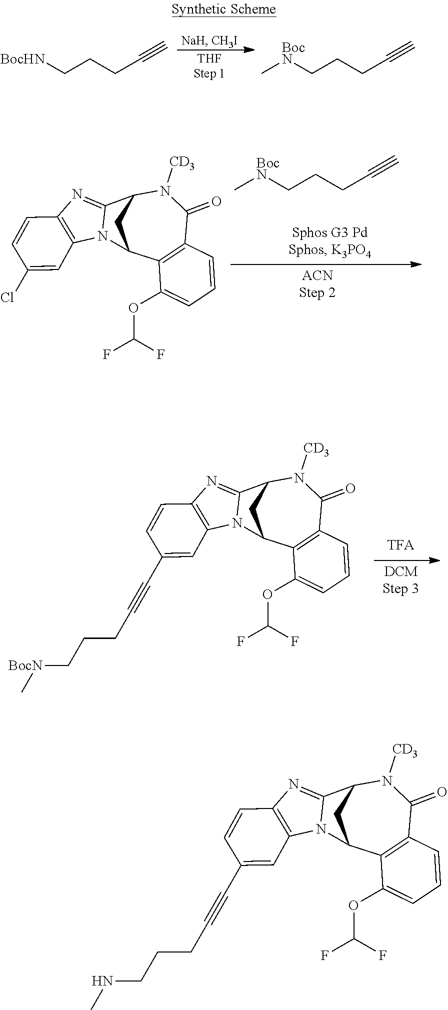

### Preparation 153A: tert-butyl N-methyl-N-(pent-4-yn-1-yl)carbamate

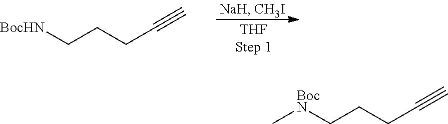

To a solution of tert-butyl N-(pent-4-yn-1-yl)carbamate (500 mg, 2.728 mmol) in THF (5 mL) was added NaH (164 mg, 4.092 mmol, 60%) at 0 degrees C. The mixture was stirred for 30 min. CH₃I (581 mg, 4.092 mmol) was added and the mixture was allowed to warm to room temperature and stirred for overnight. The reaction mixture was quenched by water and extracted with DCM (3×50 mL), dried over anhydrous Na₂SO₄. After filtration, the filtrate was concentrated under reduced pressure. The residue was purified by silica gel column chromatography, eluted with PE/EA (0-40%) to afford tert-butyl N-methyl-N-(pent-4-yn-1-yl)carbamate (320 mg, 64%) as a light yellow liquid. MS ESI calculated for C₁₁H₁₉NO₂ [M+H]⁺, 198.14. found 198.14. ¹H NMR (400 MHz, Chloroform-d) δ 3.29 (t, J=7.1 Hz, 2H), 2.84 (s, 3H), 2.23-2.14 (m, 2H), 1.94 (t, J=2.6 Hz, 1H), 1.78-1.68 (m, 2H), 1.44 (s, 9H).

### Preparation 153B: tert-butyl (5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-yn-1-yl)(methyl)carbamate

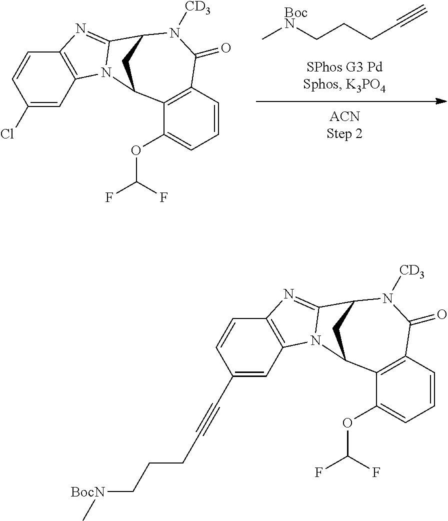

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (100 mg, 0.255 mmol) and tert-butyl N-methyl-N-(pent-4-yn-1-yl)carbamate (251 mg, 1.275 mmol) in ACN (0.5 mL) were added K₂CO₃ (176 mg, 1.275 mmol) and SPhos (21 mg, 0.051 mmol), SPhos Pd Gen.3 (40 mg, 0.051 mmol). After stirring for overnight at 80° C. under a nitrogen atmosphere, the resulting mixture was concentrated under reduced pressure. The residue was purified by silica gel column chromatography, eluted with PE/EA (0~100%) followed purified by reversed-phase flash chromatography with the following conditions: column, C18 silica gel; mobile phase, CH₃CN in water (10 mmol/L NH₄HCO₃), 50% to 70% gradient in 10 min; detector, 254 nm. This resulted in tert-butyl (5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-yn-1-yl)(methyl)carbamate (107 mg, 76%) as a white solid. MS ESI calculated for C₃₀H₂₉D₃F₂N₄O₄ [M+H]⁺, 554.26. found 554.30. ¹H NMR (400 MHz, Chloroform-d) δ 8.52-8.46 (m, 1H), 7.67-7.62 (m, 1H), 7.55-7.50 (m, 1H), 7.47-7.40 (m, 1H), 7.37-7.29 (m, 2H), 6.87 (t, J=72.6 Hz, 1H), 6.25 (d, J=7.2 Hz, 1H), 5.06 (d, J=7.4 Hz, 1H), 3.53-3.43 (m, 1H), 3.37 (t, J=7.0 Hz, 2H), 2.93-2.84 (m, 4H), 2.45 (t, J=7.0 Hz, 2H), 1.89-1.77 (m, 2H), 1.45 (s, 9H).

### Example 153: (7R,14R)-1-(difluoromethoxy)-6-(methyl-d3)-11-(5-(methylamino)pent-1-yn-1-yl)-6,7-dihydro-7,14-methanobenzo[f]benzo[4,5]imidazo[1,2-a][1,4]diazocin-5(14H)-one

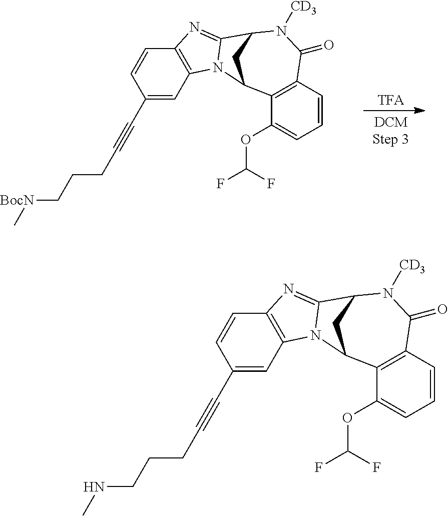 [SMILES: [2H]C([2H])([2H])N1C(=O)c2cccc(OC(F)F)c2[C@H]2C[C@@H]1c1nc3ccc(C#CCCCN(C)C(=O)OC(C)(C)C)cc3n12 ; [2H]C([2H])([2H])N1C(=O)c2cccc(OC(F)F)c2[C@H]2C[C@@H]1c1nc3ccc(C#CCCCNC)cc3n12 ; "TFA"]

To a stirred solution of tert-butyl (5-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)pent-4-yn-1-yl)(methyl)carbamate (107 mg, 0.193 mmol) in DCM (2 mL) was added TFA (0.4 mL) dropwise at room temperature. The mixture was stirred for 1 h at room temperature. The resulting mixture was concentrated under vacuum and purified by reversed-phase flash chromatography with the following conditions: column, C18 silica gel; mobile phase, CH₃CN in Water (10 mmol/L NH₄HCO₃), 25% to 50% gradient in 20 min; detector, 254 nm. This resulted in (7R,14R)-1-(difluoromethoxy)-6-(methyl-d3)-11-(5-(methylamino)pent-1-yn-1-yl)-6,7-dihydro-7,14-methanobenzo[f]benzo[4,5]imidazo[1,2-a][1,4]diazocin-5(14H)-one (32 mg, 36%) as a white solid. MS ESI calculated for C₂₅H₂₁D₃F₂N₄O₂ [M+H]⁺, 454.21. found 454.20. ¹H NMR (400 MHz, Methanol-d₄) δ 8.40-8.31 (m, 1H), 7.56-7.43 (m, 4H), 7.42-7.04 (m, 2H), 6.34 (d, J=7.2 Hz, 1H), 5.16 (d, J=7.1 Hz, 1H), 3.59-3.47 (m, 1H), 2.86 (d, J=13.8 Hz, 1H), 2.80-2.70 (m, 2H), 2.50 (t, J=7.0 Hz, 2H), 2.42 (s, 3H), 1.86-1.73 (m, 2H). ¹⁹F NMR (377 MHz, Methanol-d₄) δ −82.89, −83.34, −83.50, −83.95.

### Example 154: 1-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)-3-methylazetidine-3-carbonitrile

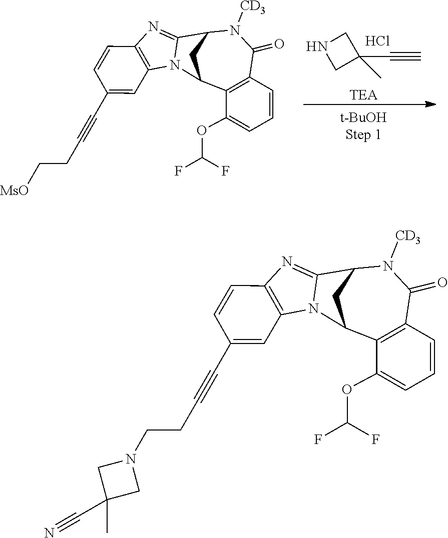

A solution of 3-ethynyl-3-methylazetidine hydrochloride (78 mg, 0.590 mmol) and TEA (0.1 mL, 0.030 mmol) in t-BuOH (0.5 mL) was stirred for 10 min at room temperature. To the above mixture was added 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (30 mg, 0.059 mmol) at room temperature. The resulting mixture was stirred for additional 16 h at 50° C. The resulting mixture was concentrated under vacuum and purified by reversed-phase flash chromatography with the following conditions: column, C18 silica gel; mobile phase, CH₃CN in water (0.1% FA), 20% to 40% gradient in 20 min; detector, 254 nm to afford 1-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)-3-methylazetidine-3-carbonitrile (10 mg, 33%) as a white solid. MS ESI calculated for C₂₈H₂₂D₃F₂N₅O₂ [M+H]⁺, 505.22. found 505.30. ¹H NMR (400 MHz, Methanol-d₄) δ 8.40-8.33 (m, 1H), 7.61-7.51 (m, 2H), 7.51-7.40 (m, 2H), 7.30-7.05 (m, 2H), 6.34 (d, J=7.1 Hz, 1H), 5.16 (d, J=7.1 Hz, 1H), 3.68 (d, J=7.6 Hz, 2H), 3.58-3.47 (m, 1H), 3.35 (d, J=7.6 Hz, 2H), 2.86 (d, J=13.9 Hz, 1H), 2.74 (t, J=6.8 Hz, 2H), 2.51 (t, J=6.8 Hz, 2H), 1.61 (s, 3H). ¹⁹F NMR (377 MHz, Methanol-d₄) δ −82.74, −83.19, −83.43, −83.88.

### Example 155: (7R,14R)-1-(difluoromethoxy)-11-((1,3-dimethylazetidin-3-yl)ethynyl)-6-(methyl-d3)-6,7-dihydro-7,14-methanobenzo[f]benzo[4,5]imidazo[1,2-a][1,4]diazocin-5(14H)-one

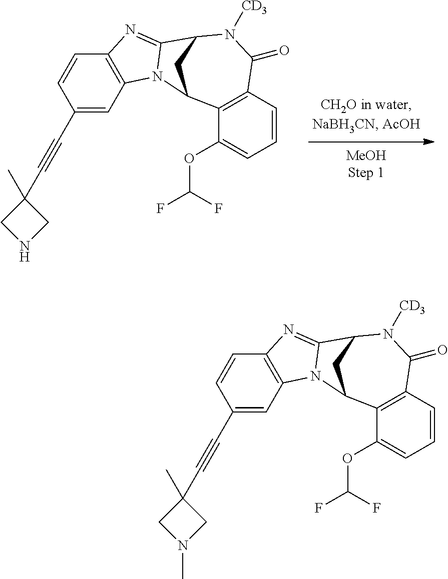 [SMILES: [2H]C([2H])([2H])N1C(=O)c2cccc(OC(F)F)c2[C@H]2C[C@@H]1c1nc3ccc(C#CC4(C)CNC4)cc3n12 ; [2H]C([2H])([2H])N1C(=O)c2cccc(OC(F)F)c2[C@H]2C[C@@H]1c1nc3ccc(C#CC4(C)CN(C)C4)cc3n12]

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (35 mg, 0.078 mmol), AcOH (14 mg, 0.234 mmol) and formaldehyde (19 mg, 0.234 mmol, 37%) in MeOH (0.5 mL) was added NaBH₃CN (15 mg, 0.234 mmol). The solution was stirred for 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 40% to 60% gradient in 20 min; detector, 254 nm. This resulted in (7R,14R)-1-(difluoromethoxy)-11-((1,3-dimethylazetidin-3-yl)ethynyl)-6-(methyl-d3)-6,7-dihydro-7,14-methanobenzo[f]benzo[4,5]imidazo[1,2-a][1,4]diazocin-5(14H)-one (25 mg, 68%) as a white solid. MS ESI calculated for C₂₆H₂₁D₃F₂N₄O₂ [M+H]⁺, 466.21. found 466.05. ¹H NMR (400 MHz, Chloroform-d) δ 8.55-8.40 (m, 1H), 7.60 (d, J=8.4 Hz, 1H), 7.50 (d, J=1.5 Hz, 1H), 7.42 (t, J=8.2 Hz, 1H), 7.34 (d, J=8.2 Hz, 1H), 7.31-7.28 (m, 1H), 6.86 (t, J=73.3 Hz, 1H), 6.19 (d, J=7.2 Hz, 1H), 4.92 (d, J=7.1 Hz, 1H), 3.59-3.20 (m, 5H), 2.85 (d, J=13.5 Hz, 1H), 2.40 (s, 3H), 1.64 (s, 3H). ¹⁹F NMR (377 MHz, Chloroform-d) δ −79.92, −80.37, −81.30, −81.74.

### Example 156: (7R,14R)-1-(difluoromethoxy)-11-((1-(2-hydroxy-2-methylpropanoyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one

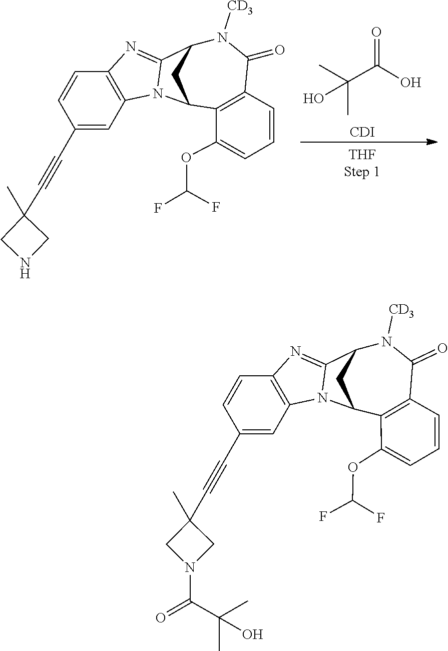

A mixture of 2-hydroxy-2-methylpropanoic acid (14 mg, 0.132 mmol) and CDI (22 mg, 0.132 mmol) in THF (0.5 mL) was stirred for 10 min at room temperature, followed by addition of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (30 mg, 0.066 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 60% gradient in 20 min; detector, 254 nm. This resulted in (7R,14R)-1-(difluoromethoxy)-11-((1-(2-hydroxy-2-methylpropanoyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one (19 mg, 54%) as a white solid. MS ESI calculated for C₂₉H₂₅D₃F₂N₄O₄[M+H]⁺, 538.23. found 538.20. ¹H NMR (400 MHz, Chloroform-d) δ 8.52-8.45 (m, 1H), 7.66 (d, J=8.5 Hz, 1H), 7.53 (s, 1H), 7.44 (t, J=8.2 Hz, 1H), 7.38-7.28 (m, 2H), 6.85 (t, J=72.7 Hz, 1H), 6.25 (d, J=7.2 Hz, 1H), 5.05 (d, J=7.0 Hz, 1H), 4.59 (d, J=8.5 Hz, 1H), 4.33-4.21 (m, 2H), 3.98 (d, J=9.5 Hz, 1H), 3.54-3.43 (m, 1H), 2.89 (d, J=13.6 Hz, 1H), 1.67 (s, 3H), 1.44 (s, 6H). ¹⁹F NMR (377 MHz, Chloroform-d) δ −80.18, −80.63, −81.04, −81.49.

### Example 157: 3-(3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylazetidin-1-yl)-2,2-dimethyl-3-oxopropanenitrile

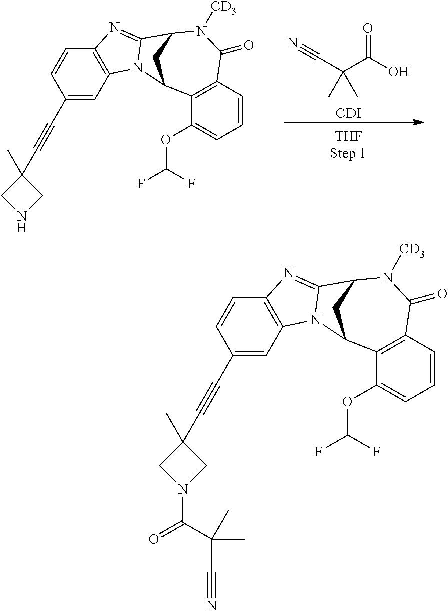

A mixture of 2-cyano-2,2-dimethylacetic acid (23 mg, 0.198 mmol) and CDI (22 mg, 0.132 mmol) in THF (0.5 mL) was stirred for 10 min at room temperature. To the above mixture was added (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (30 mg, 0.066 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in water (10 mmol/L NH₄HCO₃), 40% to 60% gradient in 20 min; detector, 254 nm. This resulted in 3-(3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylazetidin-1-yl)-2,2-dimethyl-3-oxopropanenitrile (18 mg, 51%) as a white solid. MS ESI calculated for C₃₀H₂₄D₃F₂N₅O₃ [M+H]⁺, 547.23. found 547.15. ¹H NMR (400 MHz, Chloroform-d) δ 8.53-8.44 (m, 1H), 7.64 (d, J=8.4 Hz, 1H), 7.51 (d, J=1.5 Hz, 1H), 7.43 (t, J=8.2 Hz, 1H), 7.35 (d, J=8.1 Hz, 1H), 7.31-7.28 (m, 1H), 6.86 (t, J=72.7 Hz, 1H), 6.22 (d, J=7.2 Hz, 1H), 4.97 (d, J=7.0 Hz, 1H), 4.75 (d, J=8.7 Hz, 1H), 4.45 (d, J=8.7 Hz, 1H), 4.31 (d, J=9.7 Hz, 1H), 3.99 (d, J=9.7 Hz, 1H), 3.53-3.39 (m, 1H), 2.87 (d, J=13.6 Hz, 1H), 1.69 (s, 3H), 1.59 (s, 6H). ¹⁹F NMR (377 MHz, Chloroform-d) δ −80.09, −80.53, −81.19, −81.64.

### Example 158: (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one

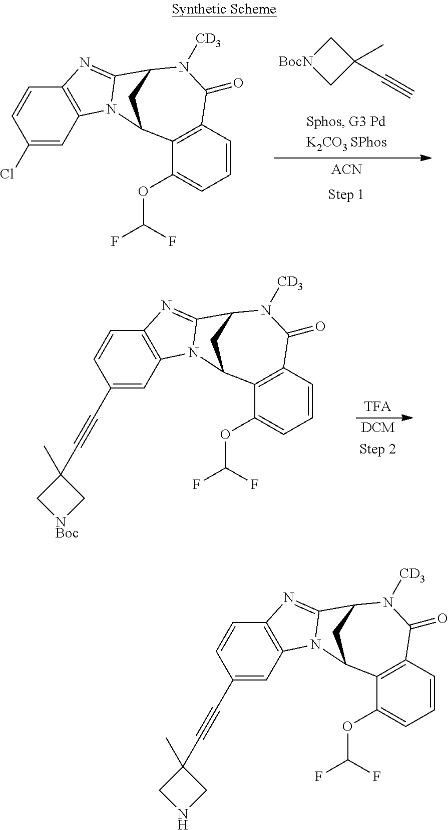

### Preparation 158A: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylazetidine-1-carboxylate

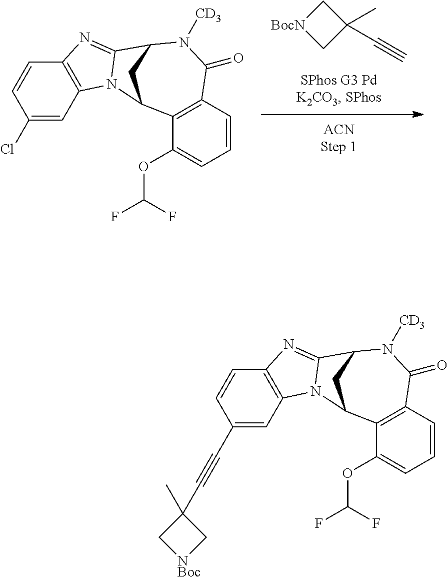 [SMILES: [2H]C([2H])([2H])N1C(=O)c2cccc(OC(F)F)c2[C@H]2C[C@@H]1c1nc3ccc(Cl)cc3n12 ; C#CC1(C)CN(C(=O)OC(C)(C)C)C1 ; [2H]C([2H])([2H])N1C(=O)c2cccc(OC(F)F)c2[C@H]2C[C@@H]1c1nc3ccc(C#CC4(C)CN(C(=O)OC(C)(C)C)C4)cc3n12]

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (500 mg, 1.273 mmol), K₂CO₃ (880 mg, 6.365 mmol), SPhos (105 mg, 0.255 mmol), SPhos Pd Gen.3 (199 mg, 0.255 mmol) and tert-butyl 3-ethynyl-3-methylazetidine-1-carboxylate (994 mg, 5.092 mmol) in CH₃CN (7 mL) was stirred for 2 h at 80° C. under nitrogen atmosphere. The resulting mixture was concentrated under reduced pressure and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 60% to 80% gradient in 20 min; detector, 254 nm. This resulted in tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylazetidine-1-carboxylate (403 mg, 57%) as a light orange solid. MS ESI calculated for C₃₀H₂₇D₃F₂N₄O₄[M+H]⁺, 552.24. found 552.20. ¹H NMR (400 MHz, Methanol-d₄) δ 8.40-8.33 (m, 1H), 7.62-7.53 (m, 2H), 7.52-7.42 (m, 2H), 7.30-7.20 (m, 2H), 6.35 (d, J=7.1 Hz, 1H), 5.17 (d, J=7.2 Hz, 1H), 4.17-4.01 (m, 2H), 3.84 (d, J=8.1 Hz, 2H), 3.58-3.45 (m, 1H), 2.87 (d, J=13.9 Hz, 1H), 1.61 (s, 3H), 1.46 (s, 9H). ¹⁹F NMR (377 MHz, Methanol-d₄) δ −83.68.

### Example 158: (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one

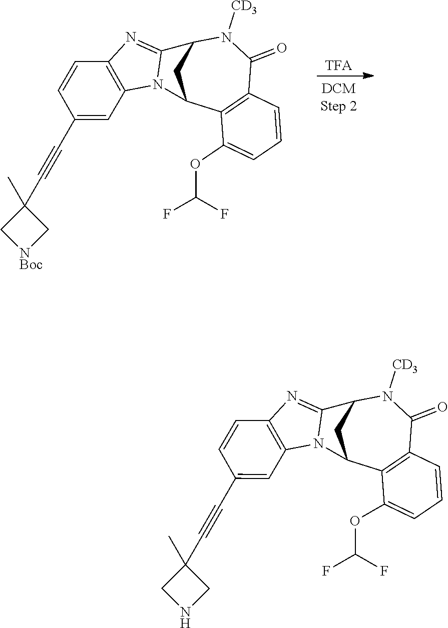 [SMILES: [2H]C([2H])([2H])N1C(=O)c2cccc(OC(F)F)c2[C@H]2C[C@@H]1c1nc3ccc(C#CC4(C)CN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4(C)CNC4)cc3n12 ; "TFA"]

To a solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylazetidine-1-carboxylate (400 mg, 0.725 mmol) in DCM (5 mL) was added TFA (1 mL) dropwise at room temperature. This reaction was stirred for 1 h at room temperature. The resulting mixture was concentrated under vacuum. The resulting mixture was diluted with saturated NaHCO₃(aq.) (20 mL), and extracted with CH₂Cl₂/MeOH (10/1) (3×20 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 50% to 70% gradient in 20 min; detector, 254 nm. This resulted in (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (253 mg, 77%) as a white solid. MS ESI calculated for C₂₅H₁₉D₃F₂N₄O₂ [M+H]⁺, 452.19. found 452.15. ¹H NMR (400 MHz, DMSO-d₆) δ 8.36-8.24 (m, 1H), 7.82-7.39 (m, 5H), 7.27-7.16 (m, 1H), 6.23 (d, J=7.1 Hz, 1H), 5.21 (d, J=7.1 Hz, 1H), 3.73 (d, J=7.1 Hz, 2H), 3.56-3.46 (m, 2H), 3.32 (d, J=7.4 Hz, 2H), 2.80 (d, J=13.8 Hz, 1H), 1.57 (s, 3H). ¹⁹F NMR (377 MHz, DMSO-d₆) δ −81.73, −82.18, −82.21, −82.65.

### Example 159: 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methylcyclobutane-1-carbonitrile

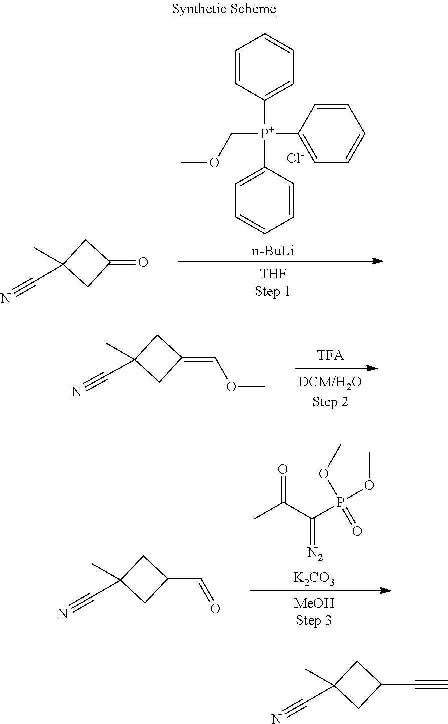

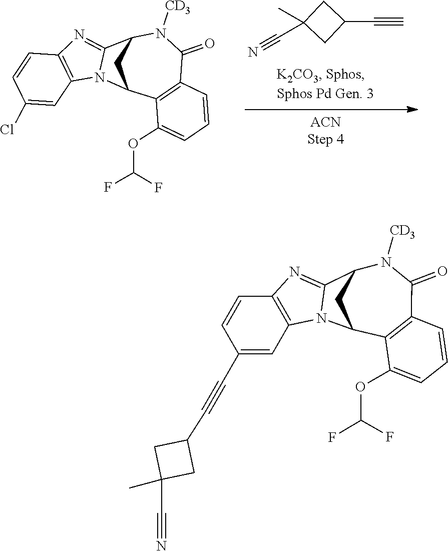

### Preparation 159A: 3-(methoxymethylidene)-1-methylcyclobutane-1-carbonitrile

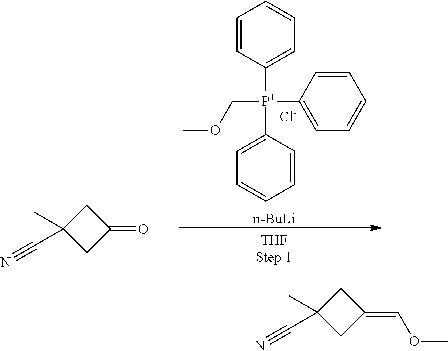

To a stirred mixture of (Methoxymethyl)triphenylphosphonium chloride (6.93 g, 20.160 mmol) in THF (116 mL) was added 2.5 M n-BuLi (8.4 mL, 20.893 mmol) at 0° C. under nitrogen atmosphere. The resulting mixture was stirred for 40 min at 0° C. under nitrogen atmosphere. To the above mixture was added 1-methyl-3-oxocyclobutane-1-carbonitrile (2.00 g, 18.327 mmol) at 0° C. The resulting mixture was stirred for additional 16 h at room temperature. The reaction was quenched with Water at room temperature. The resulting mixture was concentrated under vacuum and purified by silica gel column chromatography, eluted with PE/EA (5:1) to afford 3-(methoxymethylidene)-1-methylcyclobutane-1-carbonitrile (1.10 g, 44%) as a yellow oil. MS ESI calculated for C₈H₁₁NO [M+H]⁺, 138.08 found N/A. ¹H NMR (300 MHz, Chloroform-d) δ 5.94-5.89 (m, 1H), 3.59 (s, 3H), 3.31-3.17 (m, 2H), 2.77-2.58 (m, 2H), 1.57 (s, 3H).

### Preparation 159B: 3-formyl-1-methylcyclobutane-1-carbonitrile

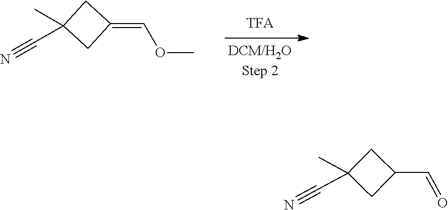

To a stirred mixture of 3-(methoxymethylidene)-1-methylcyclobutane-1-carbonitrile (1.00 g, 7.290 mmol) in H₂O (0.9 mL) and DCM (20 mL) was added TFA (7 mL) at 0° C. The resulting mixture was stirred for 1 h at room temperature. The resulting mixture was diluted with water (10 mL). The aqueous layer was extracted with CH₂Cl₂ (3×20 mL). The resulting mixture was concentrated under vacuum. This resulted in 3-formyl-1-methylcyclobutane-1-carbonitrile (1.00 g) as a yellow oil. MS ESI calculated for C₇H₉NO [M+H]⁺, 124.07 found N/A. ¹H NMR (300 MHz, Chloroform-d) δ 9.80-9.76 (m, 1H), 3.36-3.22 (m, 1H), 2.93-2.83 (m, 2H), 2.34-2.23 (m, 2H), 1.64 (s, 3H).

### Preparation 159C: 3-ethynyl-1-methylcyclobutane-1-carbonitrile

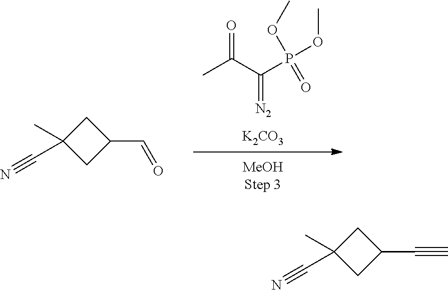

To a stirred mixture of 3-formyl-1-methylcyclobutane-1-carbonitrile (1.00 g, 8.120 mmol) and K₂CO₃ (2.24 g, 16.240 mmol) in MeOH (20 mL) was added seyferth-gilbert homologation (2.34 g, 12.180 mmol) at 0° C. under nitrogen atmosphere. The resulting mixture was stirred for 3 h at room temperature under nitrogen atmosphere. The resulting mixture was concentrated under vacuum. The residue was purified by silica gel column chromatography, eluted with PE/EA (10:1) to afford 3-ethynyl-1-methylcyclobutane-1-carbonitrile (210 mg, 22%) as a colorless oil. MS ESI calculated for C₈H₉N [M+H]⁺, 120.07 found N/A. ¹H NMR (300 MHz, Chloroform-d) δ 3.22-3.08 (m, 1H), 2.79-2.68 (m, 2H), 2.46-2.36 (m, 2H), 2.30-2.28 (m, 1H), 1.60 (s, 3H).

### Example 159: 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methylcyclobutane-1-carbonitrile

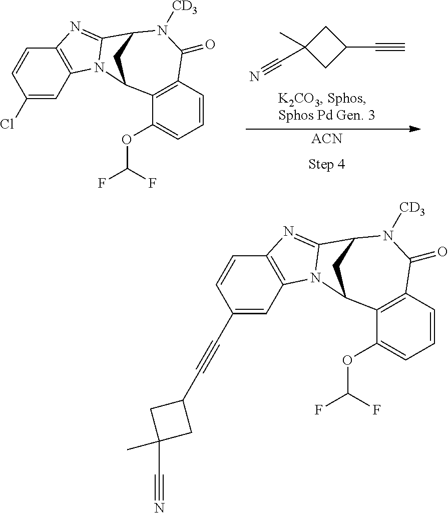

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and 3-ethynyl-1-methylcyclobutane-1-carbonitrile (73 mg, 0.612 mmol) in ACN (2 mL) were added K₂CO₃ (141 mg, 1.020 mmol), SPhos (17 mg, 0.041 mmol) and SPhos Pd Gen.3 (32 mg, 0.041 mmol) at room temperature. The resulting mixture was stirred for 4 h at 80° C. under nitrogen atmosphere. The resulting mixture was concentrated under vacuum and purified by silica gel column chromatography, eluted with CH₂Cl₂/MeOH (20:1) followed by reversed-phase flash chromatography with the following conditions: column, C18 silica gel; mobile phase, CH₃CN in Water (10 mmol/L NH₄HCO₃), 25% to 55% gradient in 30 min; detector, 254 nm. This resulted in 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1-methylcyclobutane-1-carbonitrile (33 mg, 34%) as a white solid. MS ESI calculated for C₂₇H₁₉D₃F₂N₄O₂ [M+H]⁺, 476.19. found 476.15. ¹H NMR (400 MHz, Chloroform-d) δ 8.51-8.44 (m, 1H), 7.66-7.60 (m, 1H), 7.52-7.49 (m, 1H), 7.45-7.28 (m, 3H), 7.06-6.68 (m, 1H), 6.21 (d, J=7.2 Hz, 1H), 4.95 (d, J=7.0 Hz, 1H), 3.48-3.41 (m, 1H), 3.40-3.31 (m, 1H), 2.86 (d, J=13.5 Hz, 1H), 2.83-2.76 (m, 2H), 2.51-2.39 (m, 2H), 1.58 (s, 3H). ¹⁹F NMR (377 MHz, Chloroform-d) δ −80.00, −80.45, −81.36, −81.80.

### Example 160: (7R,14R)-1-(difluoromethoxy)-6-(methyl-d3)-11-(3-(2,2,2-trifluoroethoxy)prop-1-yn-1-yl)-6,7-dihydro-7,14-methanobenzo[f]benzo[4,5]imidazo[1,2-a][1,4]diazocin-5(14H)-one

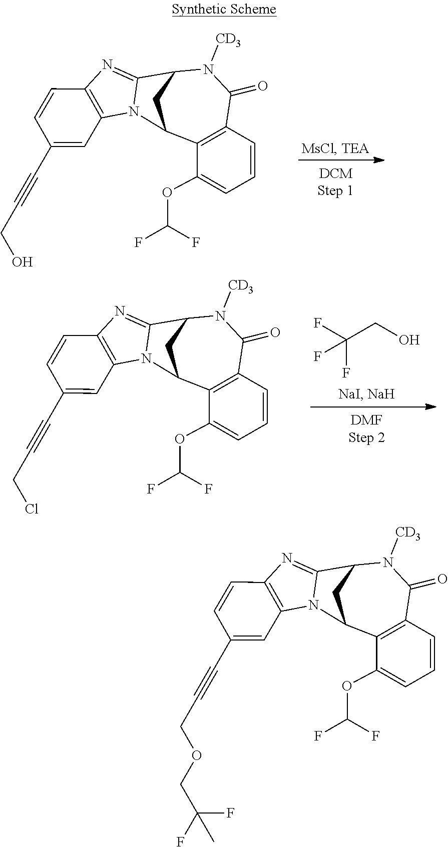

### Preparation 160A: (7R,14R)-11-(3-chloroprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

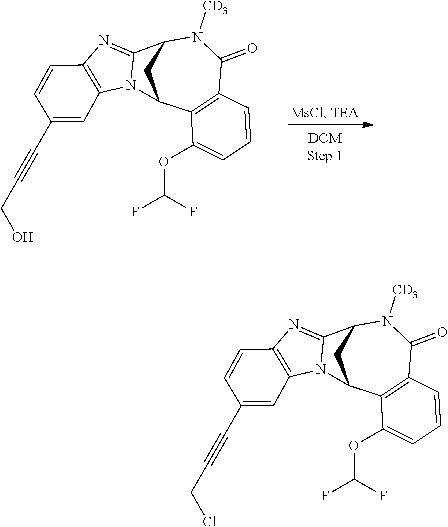 [SMILES: [2H]C([2H])([2H])N1C(=O)c2cccc(OC(F)F)c2[C@H]2C[C@@H]1c1nc3ccc(C#CCO)cc3n12 ; "MsCl, TEA" ; [2H]C([2H])([2H])N1C(=O)c2cccc(OC(F)F)c2[C@H]2C[C@@H]1c1nc3ccc(C#CCCl)cc3n12]

To a stirred solution of (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one (200 mg, 0.485 mmol) and TEA (74 mg, 0.728 mmol) in DCM (5 mL) was added MsCl (67 mg, 0.582 mmol) dropwise at 0° C. under nitrogen atmosphere. The resulting mixture was stirred for 2 h at room temperature under nitrogen atmosphere. The reaction was quenched with Water at room temperature and extracted with CH₂Cl₂ (3×20 mL). The combined organic layers were washed with brine (3×30 mL), dried over anhydrous Na₂SO₄. After filtration, the filtrate was concentrated under reduced pressure. The crude was purified by reversed-phase flash chromatography with the following conditions: column, C18 silica gel; mobile phase, CH₃CN in Water (10 mmol/L NH₄HCO₃), 30% to 50% gradient in 20 min; detector, 254 nm to afford (7R,14R)-11-(3-chloroprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (105 mg, 50%) as a white solid. MS ESI calculated for C₂₂H₁₃D₃ClF₂N₃O₂ [M+H]⁺, 431.11. found 431.20. ¹H NMR (400 MHz, Chloroform-d) δ 8.55-8.43 (m, 1H), 7.64 (d, J=8.5 Hz, 1H), 7.55 (s, 1H), 7.43 (d, J=8.2 Hz, 1H), 7.40-7.30 (m, 2H), 6.84 (t, J=73.2 Hz, 1H), 6.21 (d, J=7.2 Hz, 1H), 4.95 (d, J=7.0 Hz, 1H), 4.41 (s, 2H), 3.51-3.34 (m, 1H), 2.86 (d, J=13.6 Hz, 1H).

### Example 160: (7R,14R)-1-(difluoromethoxy)-6-(methyl-d3)-11-(3-(2,2,2-trifluoroethoxy)prop-1-yn-1-yl)-6,7-dihydro-7,14-methanobenzo[f]benzo[4,5]imidazo[1,2-a][1,4]diazocin-5(14H)-one

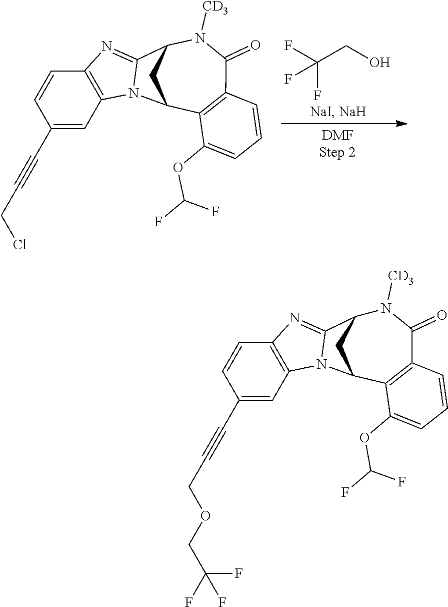

To a stirred solution of 2,2,2-trifluoroethan-1-ol (42 mg, 0.417 mmol) in DMF (2 mL) was added NaI (2 mg, 0.014 mmol) and NaH (22 mg, 0.558 mmol, 60%) at 0° C. under nitrogen atmosphere. The resulting mixture was stirred for 30 min at 0° C. under nitrogen atmosphere. To the above mixture was added (7R,14R)-11-(3-chloroprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.139 mmol) in DMF (0.5 mL) dropwise at 0° C. The resulting mixture was stirred for additional 2 h at room temperature. The reaction was quenched with Water at 0° C. The resulting mixture was extracted with EtOAc (3×30 mL). The combined organic layers were washed with brine (2×30 mL), dried over anhydrous Na₂SO₄. After filtration, the filtrate was concentrated under reduced pressure and purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254 nm to afford (7R,14R)-1-(difluoromethoxy)-6-(methyl-d3)-11-(3-(2,2,2-trifluoroethoxy)prop-1-yn-1-yl)-6,7-dihydro-7,14-methanobenzo[f]benzo[4,5]imidazo[1,2-a][1,4]diazocin-5(14H)-one (20 mg, 21%) as a white solid. MS ESI calculated for C₂₄H₁₅D₃F₅N₃O₃[M+H]⁺, 495.15. found 495.20. ¹H NMR (400 MHz, Chloroform-d) δ 8.52-8.45 (m, 1H), 7.71-7.64 (m, 1H), 7.59 (s, 1H), 7.48-7.40 (m, 1H), 7.38-7.32 (m, 2H), 6.84 (t, J=72.7 Hz, 1H), 6.25 (d, J=6.3 Hz, 1H), 5.07-4.99 (m, 1H), 4.56 (s, 2H), 4.08-3.96 (m, 2H), 3.55-3.40 (m, 1H), 2.89 (d, J=13.2 Hz, 1H). ¹⁹F NMR (377 MHz, Chloroform-d) δ −73.76, −80.23, −80.68, −81.11, −81.56.

### Example 161: (7R,14R)-11-((1-(cyclopropanecarbonyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

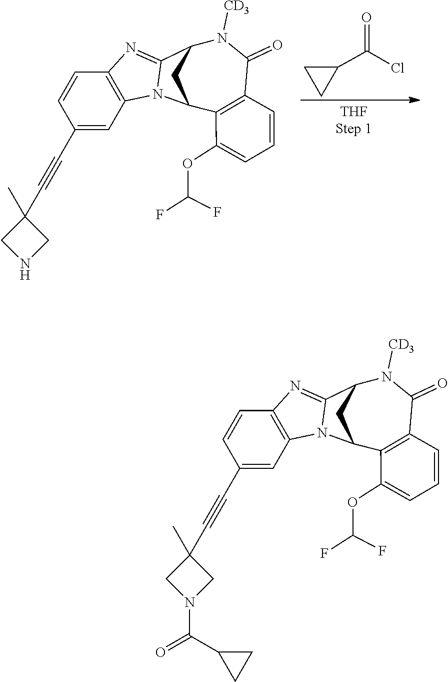

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (35 mg, 0.078 mmol) in THF (0.5 mL) was added cyclopropanecarbonyl chloride (12 mg, 0.117 mmol) at 0° C. under nitrogen atmosphere. The solution was stirred for 1 h at room temperature. The residue was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃), Mobile Phase B: MeOH; Flow rate: 60 mL/min; Gradient: 47% B to 67% B in 12 min; Wave Length: 254/200 nm; RT1(min): 9.77) to afford (7R,14R)-11-((1-(cyclopropanecarbonyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 49%) as a white solid. MS ESI calculated for C₂₉H₂₃D₃F₂N₄O₃ [M+H]⁺, 520.22. found 520.25. ¹H NMR (400 MHz, Chloroform-d) δ 8.55-8.43 (m, 1H), 7.63 (d, J=8.4 Hz, 1H), 7.50 (d, J=1.6 Hz, 1H), 7.43 (t, J=8.2 Hz, 1H), 7.35 (d, J=8.1 Hz, 1H), 7.29 (d, J=1.5 Hz, 1H), 6.85 (t, J=72.7 Hz, 1H), 6.21 (d, J=7.2 Hz, 1H), 4.94 (d, J=7.1 Hz, 1H), 4.49 (d, J=7.6 Hz, 1H), 4.24 (d, J=9.2 Hz, 1H), 4.14 (d, J=7.7 Hz, 1H), 3.93 (d, J=9.2 Hz, 1H), 3.54-3.39 (m, 1H), 2.86 (d, J=13.6 Hz, 1H), 1.68 (s, 3H), 1.49-1.37 (m, 1H), 0.99 (t, 2H), 0.82-0.72 (m, 2H). ¹⁹F NMR (377 MHz, Chloroform-d) δ −80.00, −80.44, −81.18, −81.63.

### Example 162: (7R,14R)-1-(difluoromethoxy)-6-(methyl-d3)-11-((3-methyl-1-(methylsulfonyl)azetidin-3-yl)ethynyl)-6,7-dihydro-7,14-methanobenzo[f]benzo[4,5]imidazo[1,2-a][1,4]diazocin-5(14H)-one

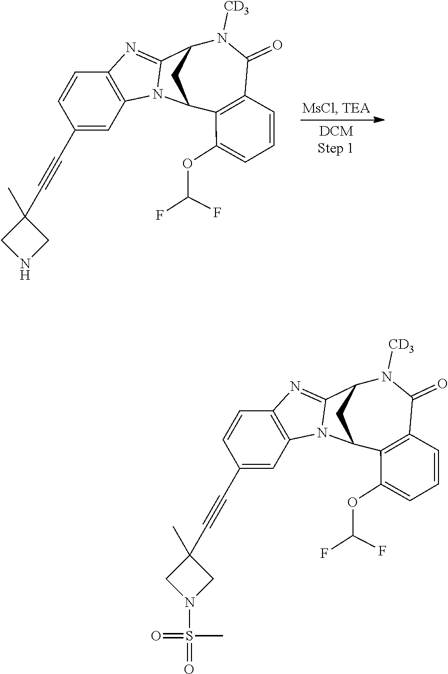

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (35 mg, 0.078 mmol) and TEA (24 mg, 0.234 mmol) in DCM (0.5 mL) was added methanesulfonyl chloride (13 mg, 0.117 mmol) at 0° C. under nitrogen atmosphere. The solution was stirred for 1 h at room temperature. The resulting mixture was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃), Mobile Phase B: CH₃CN; Flow rate: 60 mL/min; Gradient: 25% B to 45% B in 10 min; Wave Length: 254/200 nm; RT1(min): 11.33) to afford (7R,14R)-1-(difluoromethoxy)-6-(methyl-d3)-11-((3-methyl-1-(methylsulfonyl)azetidin-3-yl)ethynyl)-6,7-dihydro-7,14-methanobenzo[f]benzo[4,5]imidazo[1,2-a][1,4]diazocin-5(14H)-one (21 mg, 52%) as a white solid. MS ESI calculated for C₂₆H₂₁D₃F₂N₄O₄S [M+H]⁺, 530.17. found 530.10. ¹H NMR (400 MHz, Chloroform-d) δ 8.48 (d, J=8.2 Hz, 1H), 7.64 (d, J=8.4 Hz, 1H), 7.52 (s, 1H), 7.43 (t, J=8.2 Hz, 1H), 7.35 (d, J=8.1 Hz, 1H), 7.31-7.27 (m, 1H), 6.86 (t, J=72.8 Hz, 1H), 6.22 (d, J=7.2 Hz, 1H), 4.98 (d, J=7.1 Hz, 1H), 4.17 (d, J=7.4 Hz, 2H), 3.85 (d, J=7.4 Hz, 2H), 3.52-3.39 (m, 1H), 2.93 (s, 3H), 2.87 (d, J=13.6 Hz, 1H), 1.70 (s, 3H). ¹⁹F NMR (377 MHz, Chloroform-d) δ −80.16, −80.60, −81.03, −81.47.

### Example 163: (7R,14R)-1-(difluoromethoxy)-11-(4-(dimethylamino)but-1-yn-1-yl)-6-(methyl-d3)-6,7-dihydro-7,14-methanobenzo[f]benzo[4,5]imidazo[1,2-a][1,4]diazocin-5(14H)-one

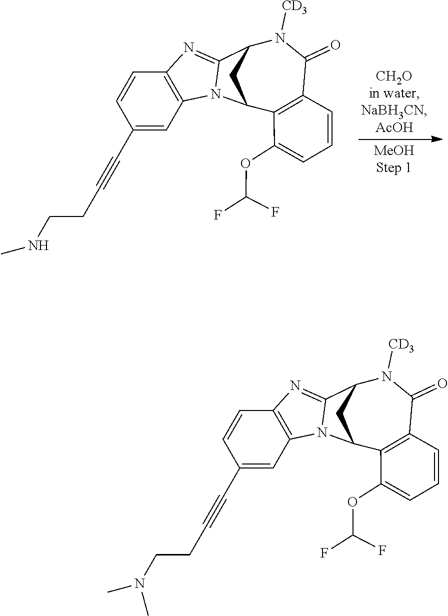 [SMILES: [2H]C([2H])([2H])N1C(=O)c2cccc(OC(F)F)c2[C@H]2C[C@@H]1c1nc3ccc(C#CCCNC)cc3n12 ; [2H]C([2H])([2H])N1C(=O)c2cccc(OC(F)F)c2[C@H]2C[C@@H]1c1nc3ccc(C#CCCN(C)C)cc3n12]

To a stirred mixture of (7R,14R)-1-(difluoromethoxy)-6-(methyl-d3)-11-(4-(methylamino)but-1-yn-1-yl)-6,7-dihydro-7,14-methanobenzo[f]benzo[4,5]imidazo[1,2-a][1,4]diazocin-5(14H)-one (30 mg, 0.068 mmol) and AcOH (12 mg, 0.204 mmol) in MeOH (2 mL) was added formaldehyde (21 mg, 0.204 mmol, 30%) at room temperature. The resulting mixture was stirred for 15 min at room temperature. To the above mixture was added NaBH₃CN (6 mg, 0.102 mmol) at room temperature. The resulting mixture was stirred for additional 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 25% to 55% gradient in 30 min; detector, 254 nm. This resulted in (7R,14R)-1-(difluoromethoxy)-11-(4-(dimethylamino)but-1-yn-1-yl)-6-(methyl-d3)-6,7-dihydro-7,14-methanobenzo[f]benzo[4,5]imidazo[1,2-a][1,4]diazocin-5(14H)-one (21 mg, 68%) as a white solid. MS ESI calculated for C₂₅H₂₁D₃F₂N₄O₂ [M+H]⁺, 454.21 found 454.20. ¹H NMR (400 MHz, Chloroform-d) δ 8.54-8.42 (m, 1H), 7.60 (d, J=8.3 Hz, 1H), 7.52-7.48 (m, 1H), 7.46-7.39 (m, 1H), 7.36-7.32 (m, 1H), 7.30-7.24 (m, 1H), 6.85 (t, J=73.4 Hz, 1H), 6.19 (d, J=7.2 Hz, 1H), 4.92 (d, J=7.0 Hz, 1H), 3.50-3.35 (m, 1H), 2.84 (d, J=13.5 Hz, 1H), 2.73-2.56 (m, 4H), 2.33 (s, 6H). ¹⁹F NMR (377 MHz, DMSO-d₆) δ −82.02.

### Example 164: (7R,14R)-1-(difluoromethoxy)-11-((1-(2-hydroxyethyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one

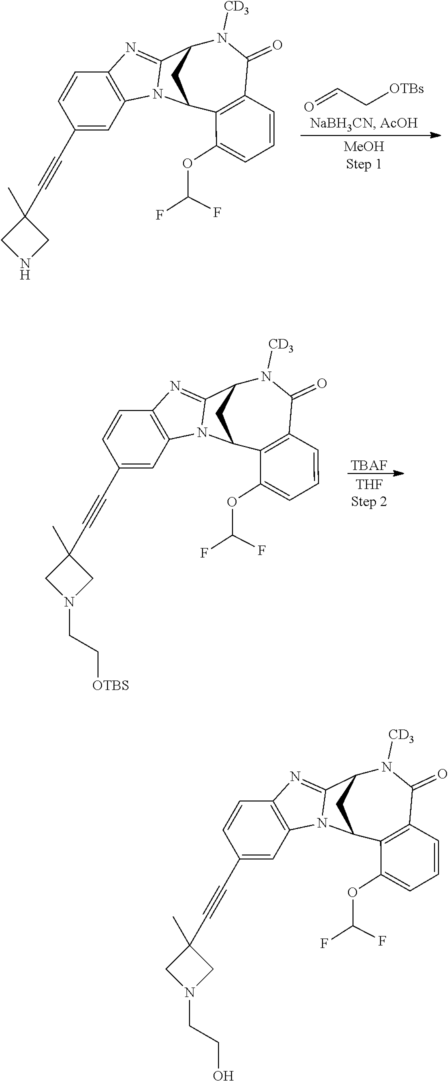

### Preparation 164A: (7R,14R)-11-((1-(2-((tert-butyldimethylsilyl)oxy)ethyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

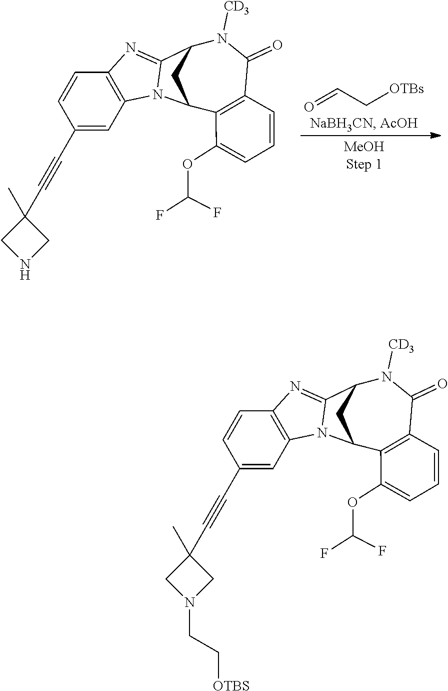

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (35 mg, 0.078 mmol), AcOH (14 mg, 0.234 mmol) and 2-[(tert-butyldimethylsilyl)oxy]acetaldehyde (41 mg, 0.233 mmol) in MeOH (0.5 mL) was added NaBH₃CN (15 mg, 0.234 mmol). The solution was stirred for 1 h at room temperature. The resulting mixture was concentrated under reduced pressure, and diluted with water (10 mL). The aqueous layer was extracted with EtOAc (3×10 mL). The combined organic layers were dried over anhydrous Na₂SO₄. After filtration, the filtrate was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254 nm. This resulted in (7R,14R)-11-((1-(2-((tert-butyldimethylsilyl)oxy)ethyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (21 mg, 44%) as a yellow green oil. MS ESI calculated for C₃₃H₃₇D₃F₂N₄O₃Si [M+H]⁺, 610.30. found 610.35. ¹H NMR (400 MHz, DMSO-d₆) δ 8.30-8.20 (m, 1H), 7.78-7.33 (m, 5H), 7.21-7.10 (m, 1H), 6.18 (d, J=7.2 Hz, 1H), 5.17 (d, J=7.1 Hz, 1H), 3.52 (t, J=5.7 Hz, 2H), 3.49-3.41 (m, 1H), 3.28-3.16 (m, 4H), 2.75 (d, J=13.8 Hz, 1H), 2.50-2.48 (m, 2H), 1.50 (s, 3H), 1.19 (s, 6H), 0.83 (s, 9H).

### Example 164: (7R,14R)-1-(difluoromethoxy)-11-((1-(2-hydroxyethyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one

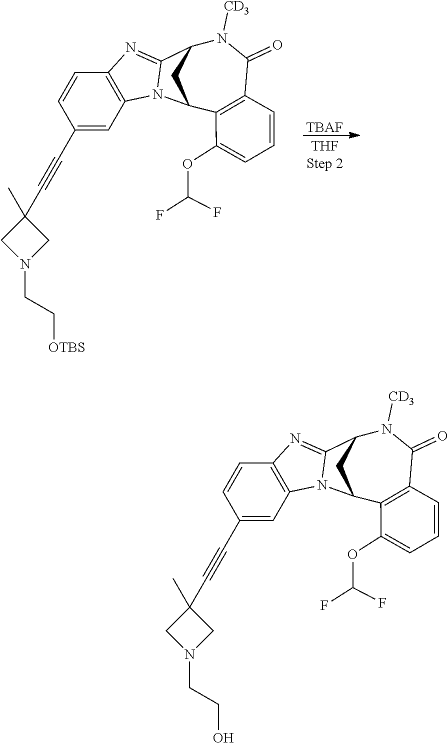

To a solution of (7R,14R)-11-((1-(2-((tert-butyldimethylsilyl)oxy)ethyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (19 mg, 0.031 mmol) in THF (0.5 mL) was added TBAF (38 uL, 0.037 mmol, 1.0 M in THF) at 0° C. The solution was stirred for 2 h at room temperature. The resulting mixture was concentrated under reduced pressure. The residue was purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in (7R,14R)-1-(difluoromethoxy)-11-((1-(2-hydroxyethyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one (10 mg, 62%) as a white solid. MS ESI calculated for C₂₇H₂₃D₃F₂N₄O₃[M+H]⁺, 496.22. found 496.20. ¹H NMR (400 MHz, Chloroform-d) δ 8.53-8.44 (m, 1H), 7.64-7.59 (m, 1H), 7.52-7.48 (m, 1H), 7.41 (t, J=8.2 Hz, 1H), 7.33 (d, J=8.1 Hz, 1H), 7.29-7.25 (m, 1H), 6.86 (t, J=72.2 Hz, 1H), 6.20 (d, J=7.2 Hz, 1H), 4.93 (d, J=7.1 Hz, 1H), 3.63-3.54 (m, 2H), 3.54-3.46 (m, 2H), 3.45-3.36 (m, 3H), 2.85 (d, J=13.6 Hz, 1H), 2.75-2.67 (m, 2H), 1.66 (s, 3H). ¹⁹F NMR (377 MHz, Chloroform-d) δ −79.98, −80.42, −81.26, −81.70.

### Example 165: (7R,14R)-1-(difluoromethoxy)-6-(methyl-d3)-11-((3-methyl-1-(1-methylcyclopropane-1-carbonyl)azetidin-3-yl)ethynyl)-6,7-dihydro-7,14-methanobenzo[f]benzo[4,5]imidazo[1,2-a][1,4]diazocin-5(14H)-one

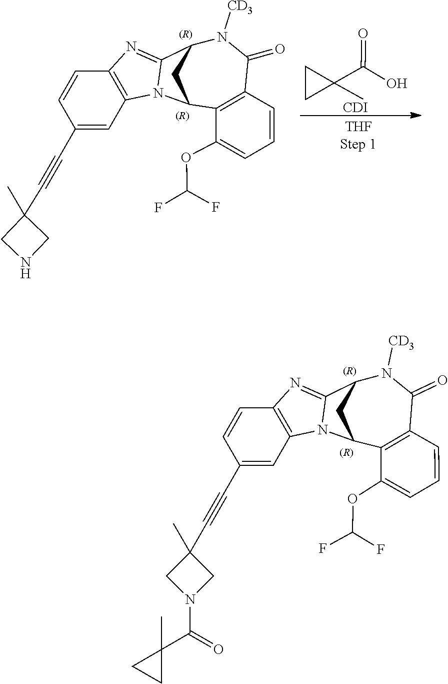

A mixture of 1-methylcyclopropane-1-carboxylic acid (12 mg, 0.124 mmol) and CDI (20 mg, 0.124 mmol) in THF (0.5 mL) was stirred for 10 min at room temperature. To the above mixture was added (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (28 mg, 0.062 mmol). The resulting mixture was stirred for additional 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 40% to 60% gradient in 20 min; detector, 254 nm. This resulted in (7R,14R)-1-(difluoromethoxy)-6-(methyl-d3)-11-((3-methyl-1-(1-methylcyclopropane-1-carbonyl)azetidin-3-yl)ethynyl)-6,7-dihydro-7,14-methanobenzo[f]benzo[4,5]imidazo[1,2-a][1,4]diazocin-5(14H)-one (24 mg, 71%) as a white solid. MS ESI calculated for C₃₀H₂₅D₃F₂N₄O₃ [M+H]⁺, 534.23. found 534.25. ¹H NMR (400 MHz, Chloroform-d) δ 8.56-8.41 (m, 1H), 7.64 (d, J=8.3 Hz, 1H), 7.51 (d, J=1.4 Hz, 1H), 7.47-7.39 (m, 1H), 7.38-7.28 (m, 2H), 7.06-6.65 (m, 1H), 6.22 (d, J=7.5 Hz, 1H), 4.99 (d, J=7.1 Hz, 1H), 4.77-3.82 (m, 4H), 3.55-3.37 (m, 1H), 2.87 (d, J=13.5 Hz, 1H), 1.66 (s, 3H), 1.31 (s, 3H), 1.15-1.05 (m, 2H), 0.62-0.48 (m, 2H). ¹⁹F NMR (377 MHz, Chloroform-d) δ −80.08, −80.52, −81.16, −81.61.

### Example 166: (7R,14R)-1-(difluoromethoxy)-6-(methyl-d3)-11-((1-(1-methylcyclopropane-1-carbonyl)azetidin-3-yl)ethynyl)-6,7-dihydro-7,14-methanobenzo[f]benzo[4,5]imidazo[1,2-a][1,4]diazocin-5(14H)-one

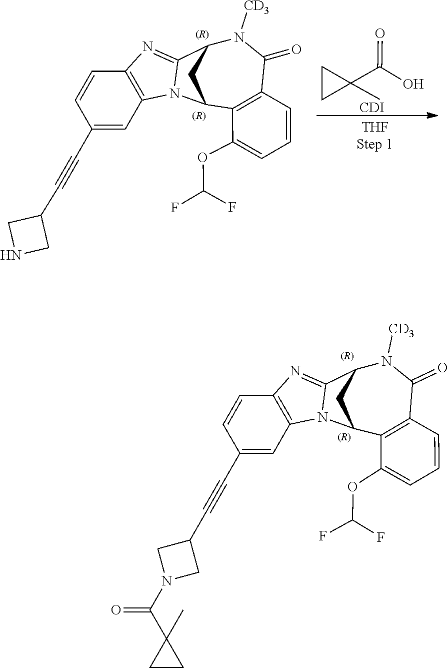

A mixture of 1-methylcyclopropane-1-carboxylic acid (18 mg, 0.182 mmol) and CDI (30 mg, 0.182 mmol) in THF (1 mL) was stirred for 10 min at room temperature. To the above mixture was added (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 60% gradient in 20 min; detector, 254 nm. This resulted in (7R,14R)-1-(difluoromethoxy)-6-(methyl-d3)-11-((1-(1-methylcyclopropane-1-carbonyl)azetidin-3-yl)ethynyl)-6,7-dihydro-7,14-methanobenzo[f]benzo[4,5]imidazo[1,2-a][1,4]diazocin-5(14H)-one (13 mg, 27%) as a white solid. MS ESI calculated for C₂₉H₂₃D₃F₂N₄O₃ [M+H]⁺, 520.22. found 520.20. ¹H NMR (400 MHz, Chloroform-d) δ 8.55-8.45 (m, 1H), 7.64 (d, J=8.4 Hz, 1H), 7.52 (d, J=1.5 Hz, 1H), 7.43 (t, J=8.2 Hz, 1H), 7.35 (d, J=8.1 Hz, 1H), 7.32-7.28 (m, 1H), 6.85 (t, J=72.7 Hz, 1H), 6.22 (d, J=7.2 Hz, 1H), 5.03-4.93 (m, 1H), 4.69-4.02 (m, 4H), 3.74-3.60 (m, 1H), 3.52-3.41 (m, 1H), 2.87 (d, J=13.6 Hz, 1H), 1.31 (s, 3H), 1.16-1.07 (m, 2H), 0.58-0.45 (m, 2H). ¹⁹F NMR (377 MHz, Chloroform-d) δ −80.01, −80.45, −81.02, −81.46.

### Example 167: (7R,14R)-1-(difluoromethoxy)-6-(methyl-d3)-11-((3-methyl-1-pivaloylazetidin-3-yl)ethynyl)-6,7-dihydro-7,14-methanobenzo[f]benzo[4,5]imidazo[1,2-a][1,4]diazocin-5(14H)-one

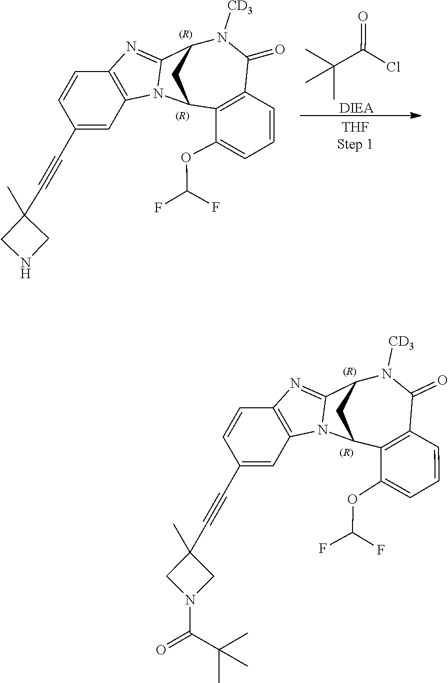

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (28 mg, 0.062 mmol) and DIEA (16 mg, 0.124 mmol) in THF (0.5 mL) was added 2,2-dimethylpropanoyl chloride (11 mg, 0.093 mmol) at 0° C. under nitrogen atmosphere. The solution was stirred for 1 h at room temperature. The reaction was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃), Mobile Phase B: CH₃CN; Flow rate: 60 mL/min; Gradient: 32% B to 52% B in 10 min; Wave Length: 254/220 nm; RT1(min): 10.02) to afford (7R,14R)-1-(difluoromethoxy)-6-(methyl-d3)-11-((3-methyl-1-pivaloylazetidin-3-yl)ethynyl)-6,7-dihydro-7,14-methanobenzo[f]benzo[4,5]imidazo[1,2-a][1,4]diazocin-5(14H)-one (18 mg, 55%) as a white solid. MS ESI calculated for C₃₀H₂₇D₃F₂N₄O₃ [M+H]⁺, 536.25. found 536.15. ¹H NMR (400 MHz, Chloroform-d) δ 8.58-8.41 (m, 1H), 7.65 (d, J=8.5 Hz, 1H), 7.51 (d, J=1.4 Hz, 1H), 7.43 (t, J=8.2 Hz, 1H), 7.38-7.28 (m, 2H), 6.85 (t, J=72.7 Hz, 1H), 6.23 (d, J=7.2 Hz, 1H), 5.00 (d, J=7.0 Hz, 1H), 4.66-3.82 (m, 4H), 3.58-3.41 (m, 1H), 2.87 (d, J=13.6 Hz, 1H), 1.65 (s, 3H), 1.22 (s, 9H). ¹⁹F NMR (377 MHz, Chloroform-d) δ −80.10, −80.54, −81.17, −81.62.

### Example 168: (7R,14R)-11-(cyclopropylethynyl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one

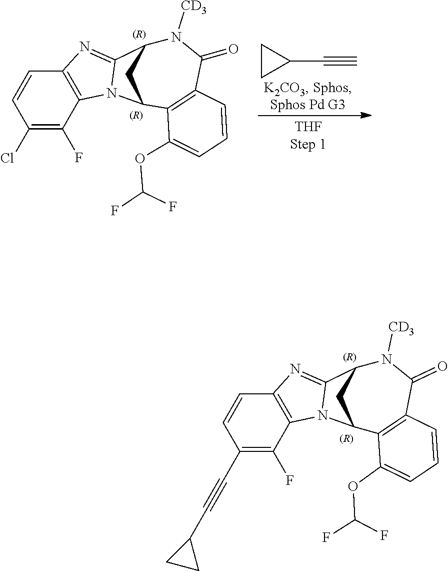

A mixture of (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (60 mg, 0.146 mmol), ethynylcyclopropane (48 mg, 0.730 mmol), SPhos (12 mg, 0.029 mmol), SPhos Pd Gen.3 (23 mg, 0.029 mmol) and K₂CO₃ (101 mg, 0.730 mmol) in CH₃CN (2 mL) was stirred for 16 h at 80° C. under nitrogen atmosphere. The resulting mixture was concentrated under vacuum. The residue was purified by silica gel column chromatography, eluted with CH₂Cl₂/MeOH (10:1) followed by reversed-phase flash chromatography with the following conditions: column, C18 silica gel; mobile phase, CH₃CN in Water (10 mmol/L NH₄HCO₃), 30% to 50% gradient in 20 min; detector, 254 nm. This resulted in (7R,14R)-11-(cyclopropylethynyl)-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (26 mg, 40%) as a white solid. MS ESI calculated for C₂₄H₁₅D₃F₃N₃O₂[M+H]⁺, 441.15. found 441.20. ¹H NMR (400 MHz, Chloroform-d) δ 8.47-8.40 (m, 1H), 7.49-7.40 (m, 2H), 7.40-7.35 (m, 1H), 7.25-7.22 (m, 1H), 6.95-6.57 (m, 1H), 6.53 (d, J=7.3 Hz, 1H), 5.15 (d, J=7.0 Hz, 1H), 3.60-3.44 (m, 1H), 2.89 (d, J=13.7 Hz, 1H), 1.56-1.46 (m, 1H), 0.99-0.80 (m, 4H). ¹⁹F NMR (377 MHz, Chloroform-d) δ −78.70, −79.15, −82.61, −82.63, −83.05, −83.07, −129.31.

### Example 169: (7R,14R)-1-(difluoromethoxy)-11-((S)-3-hydroxybut-1-yn-1-yl)-6-(methyl-d3)-6,7-dihydro-7,14-methanobenzo[f]benzo[4,5]imidazo[1,2-a][1,4]diazocin-5(14H)-one

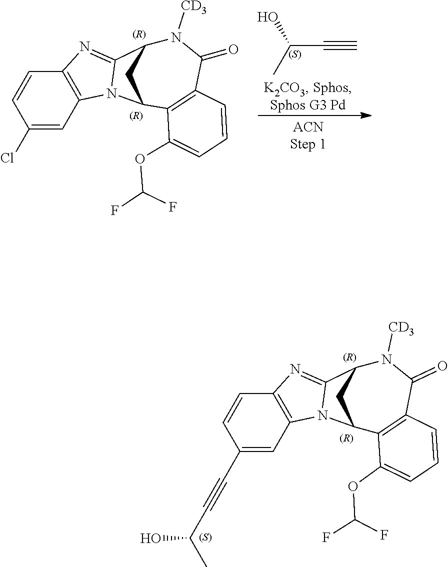

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (60 mg, 0.153 mmol), SPhos (12 mg, 0.031 mmol), SPhos Pd Gen.3 (23 mg, 0.031 mmol) and K₂CO₃ (105 mg, 0.765 mmol) in ACN (1 mL) was added (2S)-but-3-yn-2-ol (53 mg, 0.765 mmol) at room temperature. The resulting mixture was stirred for 16 h at 80° C. under nitrogen atmosphere. The resulting mixture was concentrated under reduced pressure. and purified by Prep-TLC, CH₂Cl₂/MeOH (0% to 10%) to afford followed by Prep-HPLC with the following conditions: column, C18 silica gel; mobile phase, CH₃CN in Water (10 mmol/L NH₄HCO₃), 10% to 40% gradient in 10 min; detector, 254 nm to afford (7R,14R)-1-(difluoromethoxy)-11-((S)-3-hydroxybut-1-yn-1-yl)-6-(methyl-d3)-6,7-dihydro-7,14-methanobenzo[f]benzo[4,5]imidazo[1,2-a][1,4]diazocin-5(14H)-one (9 mg, 12%) as a white solid. MS ESI calculated for C₂₃H₁₆D₃F₂N₃O₃ [M+H]⁺, 427.16. found 427.05. ¹H NMR (400 MHz, Chloroform-d) δ 8.54-8.43 (m, 1H), 7.69-7.61 (m, 1H), 7.53 (t, J=2.0 Hz, 1H), 7.47-7.40 (m, 1H), 7.35 (d, J=7.7 Hz, 1H), 7.32-7.28 (m, 1H), 6.85 (t, J=72.1 Hz, 1H), 6.26-6.18 (m, 1H), 5.00 (d, J=6.7 Hz, 1H), 4.78 (q, J=6.6 Hz, 1H), 3.56-3.37 (m, 1H), 2.94-2.79 (m, 1H), 1.63-1.51 (m, 3H). ¹⁹F NMR (377 MHz, Chloroform-d) δ −80.00, −80.44, −81.20, −81.64.

### Example 170: (7R,14R)-1-(difluoromethoxy)-12-fluoro-6-(methyl-d3)-11-(prop-1-yn-1-yl)-6,7-dihydro-7,14-methanobenzo[f]benzo[4,5]imidazo[1,2-a][1,4]diazocin-5(14H)-one

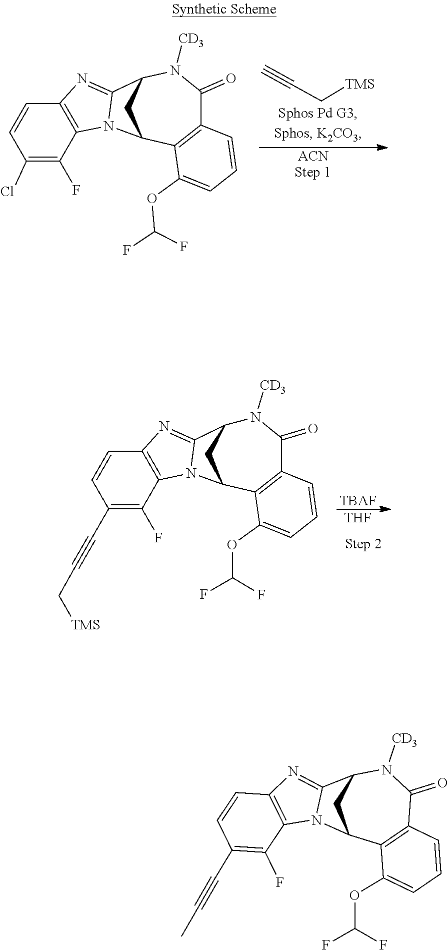

### Preparation 170A: (7R,14R)-1-(difluoromethoxy)-12-fluoro-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one

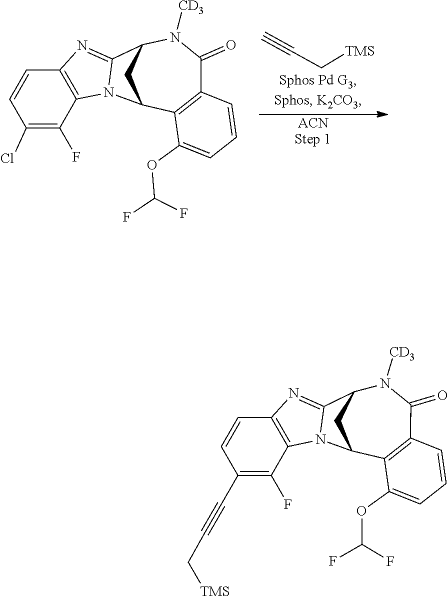

To a stirred solution of (7R,14R)-11-chloro-1-(difluoromethoxy)-12-fluoro-6-(methyl-d3)-6,7-dihydro-7,14-methanobenzo[f]benzo[4,5]imidazo[1,2-a][1,4]diazocin-5(14H)-one (100 mg, 0.243 mmol) and trimethyl(prop-2-yn-1-yl)silane (205 mg, 1.823 mmol) in ACN (2 mL) were added SPhos (20 mg, 0.049 mmol), SPhos Pd Gen.3 (28 mg, 0.036 mmol) and K₂CO₃ (168 mg, 1.215 mmol) at room temperature under nitrogen atmosphere. The resulting mixture was stirred for 2 h at 80° C. under nitrogen atmosphere. The resulting mixture was concentrated under vacuum and purified by silica gel column chromatography, eluted with CH₂Cl₂/MeOH (10/1) to afford (7R,14R)-1-(difluoromethoxy)-12-fluoro-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one (110 mg, 93%) as a light yellow liquid. MS ESI calculated for C₂₅H₂₁D₃F₃N₃O₂Si [M+H]⁺, 487.18. found 487.05.

### Example 170: (7R,14R)-1-(difluoromethoxy)-12-fluoro-6-(methyl-d3)-11-(prop-1-yn-1-yl)-6,7-dihydro-7,14-methanobenzo[f]benzo[4,5]imidazo[1,2-a][1,4]diazocin-5(14H)-one

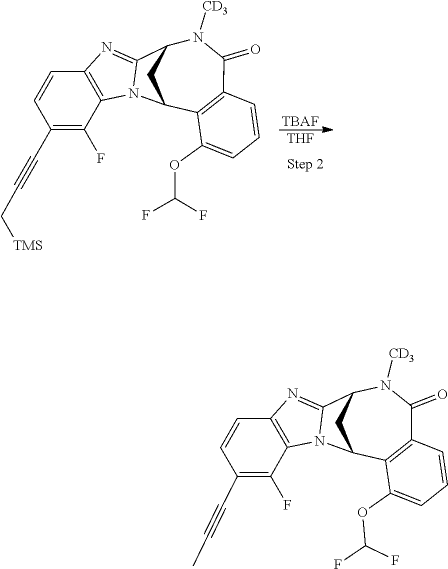

A solution of (7R,14R)-1-(difluoromethoxy)-12-fluoro-6-(methyl-d3)-11-(3-(trimethylsilyl)prop-1-yn-1-yl)-6,7-dihydro-7,14-methanobenzo[f]benzo[4,5]imidazo[1,2-a][1,4]diazocin-5(14H)-one (120 mg, 0.247 mmol) and TBAF (0.30 mL, 0.296 mmol) in THF (2 mL) was stirred for 1 h at room temperature. The resulting mixture was concentrated under reduced pressure. The residue was purified by silica gel column chromatography, eluted with DCM/MeOH (0% to 10%) followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254 nm to afford (7R,14R)-1-(difluoromethoxy)-12-fluoro-6-(methyl-d3)-11-(prop-1-yn-1-yl)-6,7-dihydro-7,14-methanobenzo[f]benzo[4,5]imidazo[1,2-a][1,4]diazocin-5(14H)-one (15 mg, 14%) as a white solid. MS ESI calculated for C22H₁₃D₃F₃N₃O₂ [M+H]⁺, 415.14. found 415.15. ¹H NMR (400 MHz, Chloroform-d) δ 8.47-8.37 (m, 1H), 7.49-7.34 (m, 3H), 7.24-7.16 (m, 1H), 6.96-6.57 (m, 1H), 6.48 (d, J=6.8 Hz, 1H), 5.04-4.89 (m, 1H), 3.56-3.39 (m, 1H), 2.85 (d, J=13.0 Hz, 1H), 2.10 (s, 3H). ¹⁹F NMR (377 MHz, Chloroform-d) δ −78.31, −78.76, −82.49, −82.93, −130.34.

### Example 171: N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)cyclopropanecarboxamide

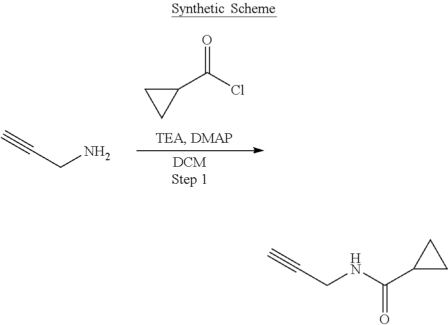

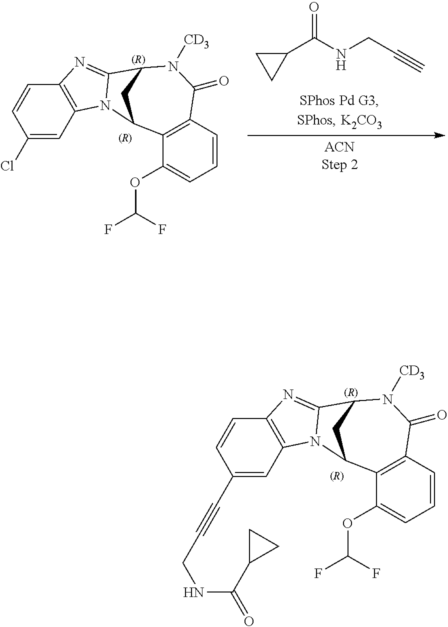

### Preparation 171A: N-(prop-2-yn-1-yl)cyclopropanecarboxamide

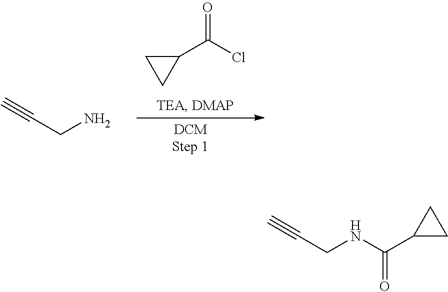

To a stirred solution of 2-propynylamine (520 mg, 9.441 mmol), DMAP (115 mg, 0.944 mmol) and TEA (2.6 mL, 18.882 mmol) in DCM (10 mL) was added cyclopropanecarbonyl chloride (1.20 g, 11.329 mmol) dropwise at 0° C. The resulting mixture was stirred for additional 6 h at room temperature. The resulting mixture was concentrated under vacuum and purified by silica gel column chromatography, eluted with PE/EA (1:1) to afford N-(prop-2-yn-1-yl)cyclopropanecarboxamide (1.00 g, 86%) as a yellow solid. MS ESI calculated for C₇H₉NO [M+H]⁺, 124.07. found N/A. ¹H NMR (400 MHz, Chloroform-d) δ 5.90 (s, 1H), 4.08 (m, 2H), 2.24 (t, J=2.6 Hz, 1H), 1.37 (m, 1H), 1.04-0.95 (m, 2H), 0.81-0.72 (m, 2H).

### Example 171: N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)cyclopropanecarboxamide

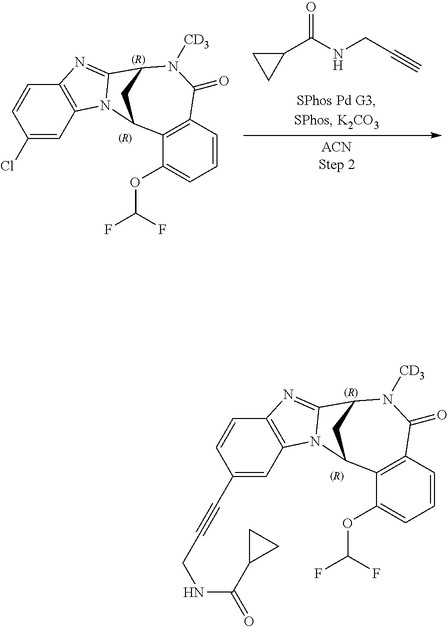

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (70 mg, 0.178 mmol), N-(prop-2-yn-1-yl)cyclopropanecarboxamide (109 mg, 0.890 mmol), SPhos (14 mg, 0.036 mmol), SPhos Pd Gen.3 (27 mg, 0.036 mmol) and K₂CO₃ (123 mg, 0.890 mmol) in ACN (2 mL) was stirred for 16 h at 80° C. under nitrogen atmosphere. The resulting mixture was concentrated under vacuum. The residue was purified by silica gel column chromatography, eluted with CH₂Cl₂/MeOH (10:1) followed by reversed-phase flash chromatography with the following conditions: column, C18 silica gel; mobile phase, CH₃CN in Water (10 mmol/L NH₄HCO₃), 30% to 50% gradient in 20 min; detector, 254 nm. This resulted in N-(3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl)cyclopropanecarboxamide (18 mg, 20%) as a white solid. MS ESI calculated for C₂₆H₁₉D₃F₂N₄O₃ [M+H]⁺, 480.18. found 480.25. ¹H NMR (400 MHz, Chloroform-d) δ 8.54-8.44 (m, 1H), 7.65 (d, J=8.4 Hz, 1H), 7.56 (s, 1H), 7.45 (t, J=8.2 Hz, 1H), 7.41-7.31 (m, 2H), 6.87 (t, J=72.5 Hz, 1H), 6.35-6.25 (m, 1H), 6.08-5.91 (m, 1H), 5.23-5.07 (m, 1H), 4.33 (d, J=5.1 Hz, 2H), 3.64-3.44 (m, 1H), 2.91 (d, J=10.3 Hz, 1H), 1.45-1.35 (m, 1H), 1.06-0.96 (m, 2H), 0.84-0.66 (m, 2H). ¹⁹F NMR (377 MHz, Chloroform-d) δ −80.11, −80.55, −81.13, −81.57.

### Example 172: (7R,14R)-11-(3-chloroprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

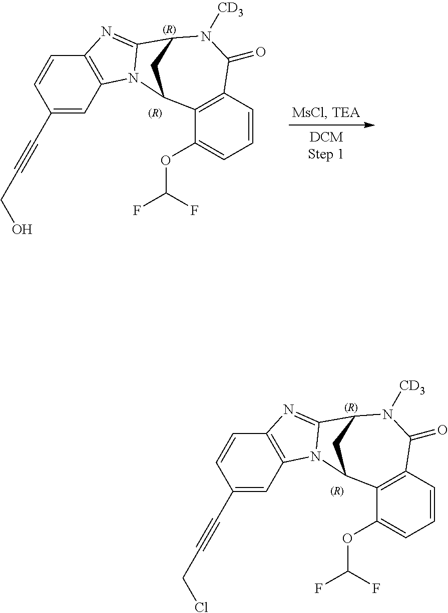

To a stirred solution of (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one (200 mg, 0.485 mmol) and TEA (74 mg, 0.728 mmol) in DCM (5 mL) was added MsCl (67 mg, 0.582 mmol) dropwise at 0° C. under nitrogen atmosphere. The resulting mixture was stirred for 2 h at room temperature under nitrogen atmosphere. The reaction was quenched with water at room temperature. The resulting mixture was extracted with CH₂Cl₂ (3×20 mL). The combined organic layers were washed with brine (1×30 mL), dried over anhydrous Na₂SO₄. After filtration, the filtrate was concentrated under reduced pressure. The crude was purified by reversed-phase flash chromatography with the following conditions: column, C18 silica gel; mobile phase, CH₃CN in water (10 mmol/L NH₄HCO₃), 30% to 50% gradient in 20 min; detector, 254 nm to afford (7R,14R)-11-(3-chloroprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (105 mg, 50%) as a white solid. MS ESI calculated for C₂₂H₁₃D₃ClF₂N₃O₂ [M+H]⁺, 431.11. found 431.20. ¹H NMR (400 MHz, Chloroform-d) δ 8.51-8.44 (m, 1H), 7.69-7.61 (m, 1H), 7.55 (s, 1H), 7.47-7.38 (m, 1H), 7.38-7.30 (m, 2H), 6.84 (t, J=73.2 Hz, 1H), 6.21 (d, J=7.2 Hz, 1H), 4.95 (d, J=7.0 Hz, 1H), 4.41 (s, 2H), 3.53-3.36 (m, 1H), 2.86 (d, J=13.6 Hz, 1H). ¹⁹F NMR (377 MHz, Chloroform-d) δ −79.94, −80.38, −81.13, −81.57.

### Example 173: (7R,14R)-11-((1-acetylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

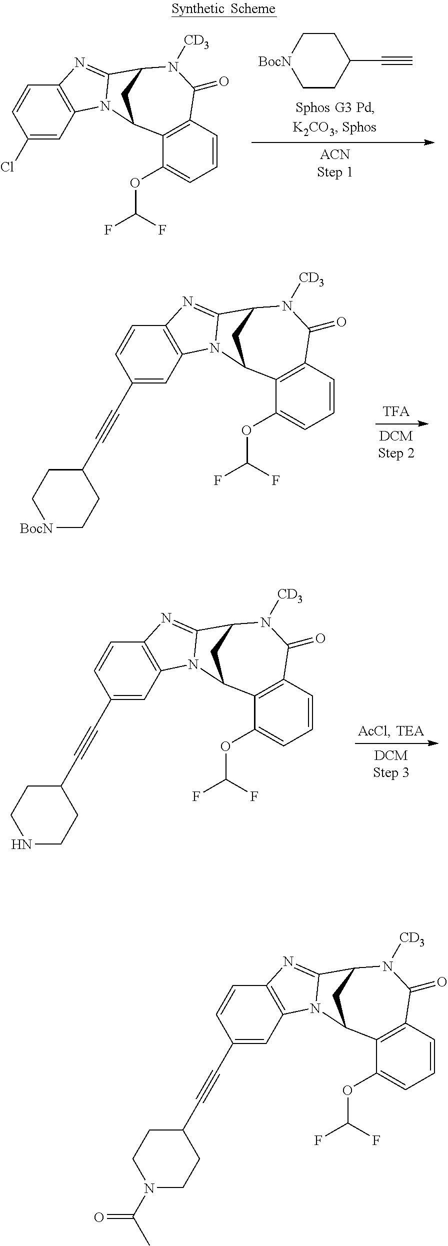

### Preparation 173A: tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate

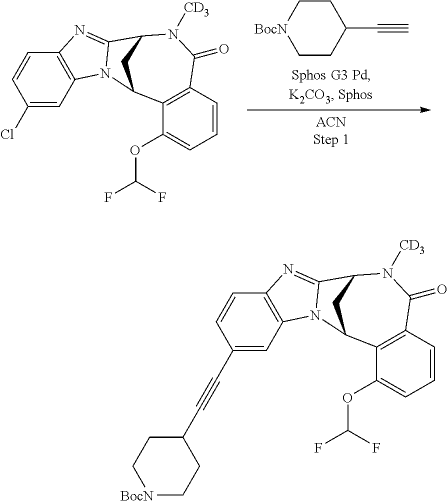

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (400 mg, 1.018 mmol) and tert-butyl 4-ethynylpiperidine-1-carboxylate (1065.60 mg, 5.090 mmol) in ACN (25 mL) were added K₂CO₃ (703.67 mg, 5.090 mmol), SPhos (83.61 mg, 0.204 mmol) and SPhos Pd Gen.3 (158.91 mg, 0.204 mmol) at room temperature. The resulting mixture was stirred for 4 h at 80° C. under nitrogen atmosphere. The reaction was quenched by the addition of water (10 mL) at room temperature. The resulting mixture was extracted with EtOAc (3×10 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 45% to 60% gradient in 20 min; detector, 254 nm to afford tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (500 mg, 85%) as a white solid. MS ESI calculated for C₃₁H₂₉D₃F₂N₄O₄[M+H]⁺, 566.26 found 566.20. ¹⁹F NMR (377 MHz, Chloroform-d) δ −79.93, −80.53, −81.13, −81.72.

### Preparation 173B: (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one

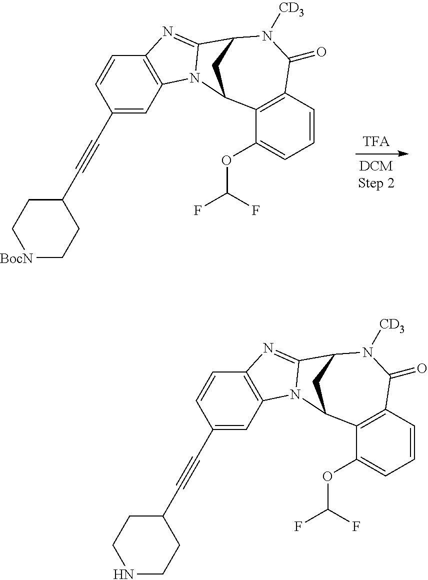 [SMILES: [2H]C([2H])([2H])N1C(=O)c2cccc(OC(F)F)c2[C@H]2C[C@@H]1c1nc3ccc(C#CC4CCN(C(=O)OC(C)(C)C)CC4)cc3n12 ; "DCMStep 2" ; [2H]C([2H])([2H])N1C(=O)c2cccc(OC(F)F)c2[C@H]2C[C@@H]1c1nc3ccc(C#CC4CCNCC4)cc3n12 ; "TFA"]

To a solution of tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (500 mg, 0.884 mmol) in DCM (20 mL) was added TFA (4 mL). The reaction was stirred for 1 h at room temperature. The resulting mixture was concentrated under reduced pressure. The mixture was neutralized to pH 7 with saturated NH₄Cl (aq.). The resulting mixture was extracted with EtOAc (3×50 mL). The combined organic layers were concentrated under reduced pressure. This resulted in (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (0.8 g, crude) as a yellow oil, which was used in the next step directly without further purification. MS ESI calculated for C₂₆H₂₁D₃F₂N₄O₂ [M+H]⁺, 466.21. found 466.20. ¹H NMR (400 MHz, Chloroform-d) δ 8.54-8.41 (m, 1H), 7.60 (d, J=8.4 Hz, 1H), 7.50 (s, 1H), 7.45-7.37 (m, 1H), 7.33 (d, J=8.1 Hz, 1H), 7.30-7.27 (m, 1H), 7.05-6.66 (m, 1H), 6.28-6.14 (m, 1H), 4.97-4.86 (m, 1H), 3.49-3.38 (m, 1H), 3.22-3.08 (m, 2H), 2.90-2.67 (m, 4H), 1.97-1.85 (m, 2H), 1.79-1.57 (m, 2H). ¹⁹F NMR (377 MHz, Chloroform-d) δ −79.97, −80.41, −81.27, −81.72.

### Example 173: (7R,14R)-11-((1-acetylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

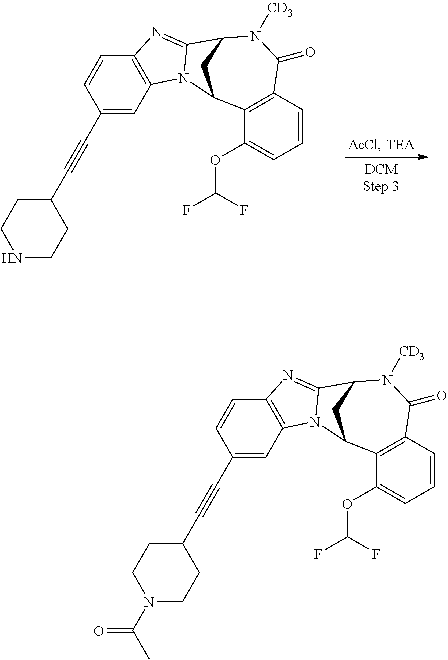 [SMILES: [2H]C([2H])([2H])N1C(=O)c2cccc(OC(F)F)c2[C@H]2C[C@@H]1c1nc3ccc(C#CC4CCNCC4)cc3n12 ; "AcCl, TEA" ; "DCMStep 3" ; [2H]C([2H])([2H])N1C(=O)c2cccc(OC(F)F)c2[C@H]2C[C@@H]1c1nc3ccc(C#CC4CCN(C(C)=O)CC4)cc3n12]

A solution of (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (40 mg, 0.086 mmol) and TEA (43 mg, 0.430 mmol) in DCM (8 mL) was stirred for 30 min at room temperature. To the above mixture was added acetyl chloride (13 mg, 0.172 mmol) at room temperature. The resulting mixture was stirred for additional 1 h at room temperature. The resulting mixture was concentrated under reduced pressure and purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 24% B to 39% B in 10 min; Wave Length: 254 nm; RT1(min): 11.649) to afford (7R,14R)-11-((1-acetylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (19 mg, 43%) as a white solid. MS ESI calculated for C₂₈H₂₃D₃F₂N₄O₃[M+H]⁺, 508.22. found 508.25. ¹H NMR (400 MHz, Chloroform-d) δ 8.53-8.42 (m, 1H), 7.64-7.58 (m, 1H), 7.52-7.49 (m, 1H), 7.42 (t, J=8.2 Hz, 1H), 7.35-7.30 (m, 1H), 7.28-7.24 (m, 1H), 6.84 (t, J=72.5 Hz, 1H), 6.20 (d, J=7.2 Hz, 1H), 4.93 (d, J=7.1 Hz, 1H), 3.97-3.86 (m, 1H), 3.77-3.68 (m, 1H), 3.56-3.32 (m, 3H), 2.96-2.81 (m, 2H), 2.11 (s, 3H), 1.99-1.85 (m, 2H), 1.81-1.69 (m, 2H). ¹⁹F NMR (377 MHz, Chloroform-d) δ −80.06, −80.07, −80.51, −80.52, −81.02, −81.04, −81.47, −81.49.

### Example 174: (7R,14R)-11-((1-(cyclopropanecarbonyl)piperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

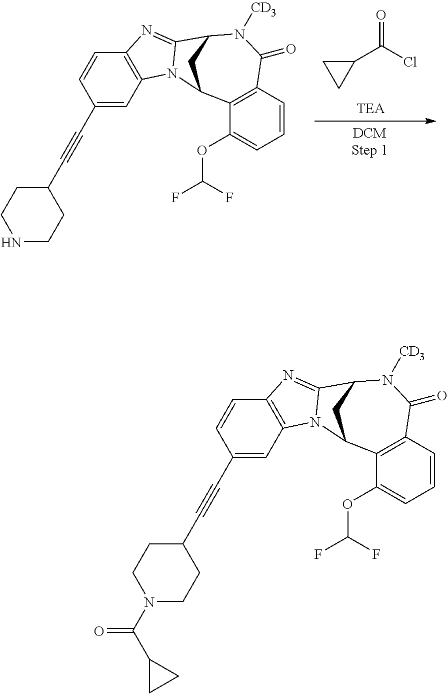

A solution of (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (40 mg, 0.086 mmol) and TEA (43 mg, 0.430 mmol) in DCM (8 mL) was stirred for 30 min at room temperature. To the above mixture was added cyclopropanecarbonyl chloride (18 mg, 0.172 mmol) at room temperature. The resulting mixture was stirred for additional 1 h at room temperature. The resulting mixture was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: ACN; Flow rate: 60 mL/min; Gradient: 28% B to 48% B in 10 min; Wave Length: 254 nm/220 nm; RT1(min): 10.5) to afford (7R,14R)-11-((1-(cyclopropanecarbonyl)piperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (21 mg, 45%) as a white solid. MS ESI calculated for C₃₀H₂₅D₃F₂N₄O₃ [M+H]⁺, 534.23 found 534.35. ¹H NMR (400 MHz, Chloroform-d) δ 8.53-8.46 (m, 1H), 7.63 (d, J=8.5 Hz, 1H), 7.52 (d, J=1.5 Hz, 1H), 7.43 (t, J=8.2 Hz, 1H), 7.36-7.27 (m, 2H), 6.84 (t, J=72.8 Hz, 1H), 6.22 (d, J=7.2 Hz, 1H), 4.98 (d, J=7.1 Hz, 1H), 4.01-3.90 (m, 2H), 3.65-3.39 (m, 3H), 2.97-2.81 (m, 2H), 2.03-1.77 (m, 5H), 1.06-0.95 (m, 2H), 0.80-0.68 (m, 2H). ¹⁹F NMR (377 MHz, Chloroform-d) δ −80.09, −80.53, −81.09, −81.53.

### Example 175: (7R,14R)-11-(cyclopropylethynyl)-1-(difluoromethoxy)-9-fluoro-6-(methyl-d3)-6,7-dihydro-7,14-methanobenzo[f]benzo[4,5]imidazo[1,2-a][1,4]diazocin-5(14H)-one

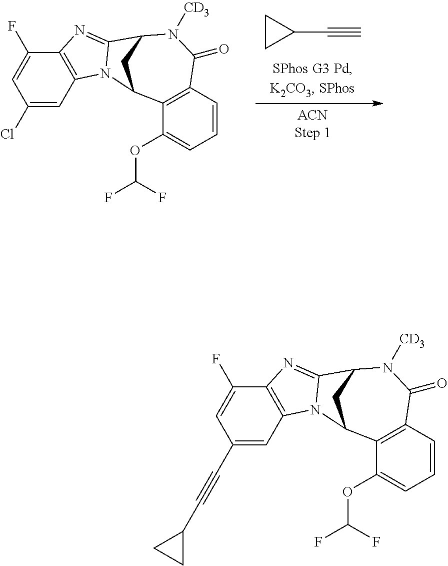

To a stirred solution of (7R,14R)-11-chloro-1-(difluoromethoxy)-9-fluoro-6-(methyl-d3)-6,7-dihydro-7,14-methanobenzo[f]benzo[4,5]imidazo[1,2-a][1,4]diazocin-5(14H)-one (50 mg, 0.122 mmol) and ethynylcyclopropane (40 mg, 0.610 mmol) in ACN (5 mL) were added K₂CO₃ (84 mg, 0.610 mmol) and SPhos Pd Gen.3 (19 mg, 0.024 mmol) at room temperature. The resulting mixture was stirred for 16 h at 80° C. under nitrogen atmosphere. The reaction was diluted with water (30 mL) and extracted with EtOAc (3×10 mL). The combined organic layers were washed with brine (2×10 mL), dried over anhydrous Na₂SO₄. After filtration, the filtrate was concentrated under reduced pressure. The crude product (40 mg) was purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min mL/min; Gradient: 36% B to 51% B in 10 min; Wave Length: 254 nm; RT1(min): 11.81) to afford (7R,14R)-11-(cyclopropylethynyl)-1-(difluoromethoxy)-9-fluoro-6-(methyl-d3)-6,7-dihydro-7,14-methanobenzo[f]benzo[4,5]imidazo[1,2-a][1,4]diazocin-5(14H)-one (23 mg, 42%) as a white solid. MS ESI calculated for C₂₄H₁₅D₃F₃N₃O₂[M+H]⁺, 441.15. found 441.00. ¹H NMR (400 MHz, Chloroform-d) δ 8.35-8.23 (m, 1H), 7.80-7.37 (m, 3H), 7.24 (d, J=1.3 Hz, 1H), 7.09-6.97 (m, 1H), 6.23 (d, J=7.1 Hz, 1H), 5.23 (d, J=7.1 Hz, 1H), 3.59-3.44 (m, 1H), 2.82 (d, J=13.8 Hz, 1H), 1.64-1.50 (m, 1H), 0.93-0.86 (m, 2H), 0.77-0.62 (m, 2H). ¹⁹F NMR (377 MHz, Chloroform-d) δ −79.94, −80.39, −81.19, −81.63, −127.07.

### Example 176: (7R,14R)-11-(3-cyclobutoxyprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

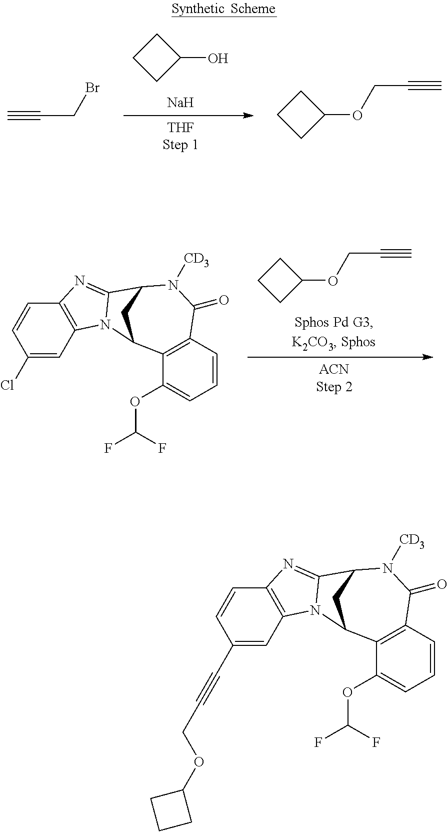

### Preparation 176A: (prop-2-yn-1-yloxy)cyclobutane

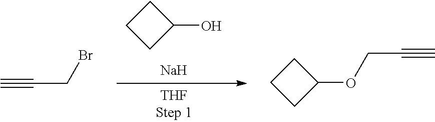

To a stirred solution of NaH (3.00 g, 74.889 mmol, 60%) in THF (300 mL) was added cyclobutanol (3.00 g, 41.605 mmol) at 0° C. The resulting mixture was stirred for 30 min at 60° C. To the above mixture was added 3-bromoprop-1-yne (5.94 g, 49.926 mmol) at room temperature. The resulting mixture was stirred for additional 4 h at 60° C. The mixture was allowed to cool down to room temperature. The reaction was quenched with Water at 0° C. The resulting mixture was extracted with CH₂Cl₂ (3×300 mL). The combined organic layers were washed with brine (1×200 mL), dried over anhydrous Na₂SO₄. After filtration, the filtrate was concentrated under reduced pressure. The residue was purified by silica gel column chromatography, eluted with PE to afford (prop-2-yn-1-yloxy)cyclobutane (3.00 g, crude) as a light yellow liquid. MS ESI calculated for C₇H₁₀O [M+H]⁺, 111.07. found N/A. ¹H NMR (400 MHz, Chloroform-d) δ 4.16-4.07 (m, 1H), 4.05 (d, J=2.4 Hz, 2H), 2.39 (t, J=2.4 Hz, 1H), 2.27-2.18 (m, 2H), 2.03-1.91 (m, 2H), 1.76-1.62 (m, 1H), 1.62-1.44 (m, 1H).

### Example 176: (7R,14R)-11-(3-cyclobutoxyprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

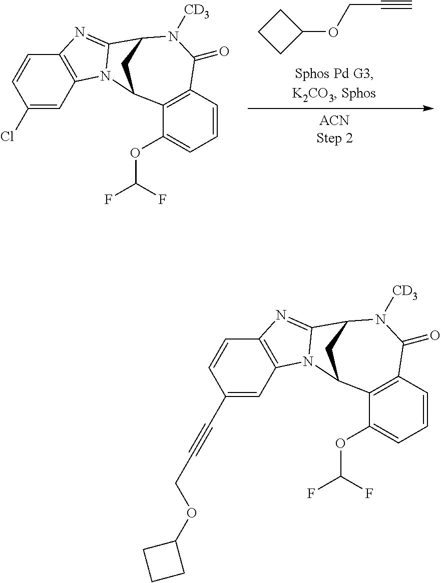

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol) and (prop-2-yn-1-yloxy)cyclobutane (112 mg, 1.020 mmol) in ACN (1.0 mL) were added SPhos Pd Gen.3 (32 mg, 0.041 mmol) and SPhos (17 mg, 0.041 mmol) at room temperature under nitrogen atmosphere. The resulting mixture was stirred for 3 h at 80° C. under nitrogen atmosphere. The mixture was allowed to cool down to room temperature. The resulting mixture was filtered, the filter cake was washed with CH₂Cl₂/MeOH (10/1, 3×5 mL). The filtrate was concentrated under reduced pressure. The residue was purified by Prep-TLC (CH₂Cl₂/EA 10/1) followed by reversed-phase flash chromatography with the following conditions: column, C18 silica gel; mobile phase, CH₃CN in Water (10 mmol/L NH₄HCO₃), 40% to 60% gradient in 20 min; detector, 254/220 nm to afford (7R,14R)-11-(3-cyclobutoxyprop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (13 mg, 14%) as a white solid. MS ESI calculated for C₂₆H₂₀D₃F₂N₃O₃[M+H]⁺, 467.19. found 467.20. ¹H NMR (400 MHz, Chloroform-d) δ 8.51-8.47 (m, 1H), 7.69-7.63 (m, 1H), 7.57 (s, 1H), 7.47-7.40 (m, 1H), 7.39-7.32 (m, 2H), 6.84 (t, J=72.7 Hz, 1H), 6.24 (d, J=6.8 Hz, 1H), 5.03 (d, J=6.6 Hz, 1H), 4.30 (s, 2H), 4.22-4.13 (m, 1H), 3.52-3.41 (m, 1H), 2.88 (d, J=13.4 Hz, 1H), 2.32-2.24 (m, 2H), 2.09-1.98 (m, 2H), 1.80-1.70 (m, 1H), 1.63-1.48 (m, 1H). ¹⁹F NMR (377 MHz, Chloroform-d) δ −80.03, −80.47, −81.10, −81.54.

### Example 177: (7R,14R)-1-(difluoromethoxy)-11-((3-hydroxy-3-methylcyclobutyl)ethynyl)-6-(methyl-d3)-6,7-dihydro-7,14-methanobenzo[f]benzo[4,5]imidazo[1,2-a][1,4]diazocin-5(14H)-one

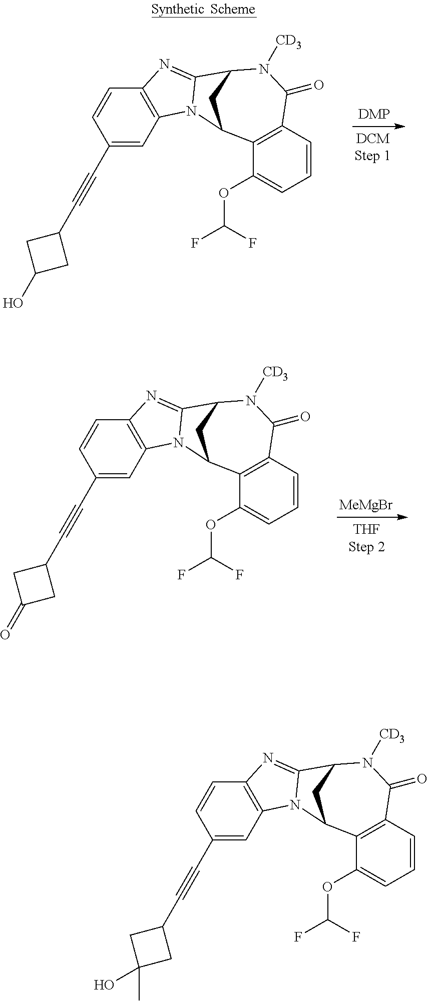

### Preparation 177A: (7R,14R)-1-(difluoromethoxy)-6-(methyl-d3)-11-((3-oxocyclobutyl)ethynyl)-6,7-dihydro-7,14-methanobenzo[f]benzo[4,5]imidazo[1,2-a][1,4]diazocin-5(14H)-one

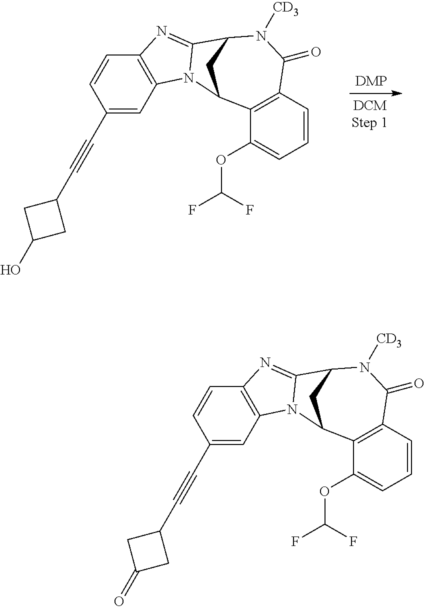

A solution of (7R,14R)-1-(difluoromethoxy)-11-((3-hydroxycyclobutyl)ethynyl)-6-(methyl-d3)-6,7-dihydro-7,14-methanobenzo[f]benzo[4,5]imidazo[1,2-a][1,4]diazocin-5(14H)-one (40 mg, 0.088 mmol) and DMP (56 mg, 0.132 mmol) in DCM (1 mL) was stirred for 2 h at room temperature. The reaction was quenched with Na₂S₂O₃ (aq.) and NaHCO₃(aq) at room temperature. The aqueous layer was extracted with CH₂Cl₂ (3×10 mL). The residue was purified by silica gel column chromatography, eluted with CH₂Cl₂/MeOH (20:1) to afford (7R,14R)-1-(difluoromethoxy)-6-(methyl-d3)-11-((3-oxocyclobutyl)ethynyl)-6,7-dihydro-7,14-methanobenzo[f]benzo[4,5]imidazo[1,2-a][1,4]diazocin-5(14H)-one (30 mg, 75%) as a white solid. MS ESI calculated for C₂₅H₁₆D₃F₂N₃O₃ [M+H]⁺, 451.16. found 451.25.

### Example 177: (7R,14R)-1-(difluoromethoxy)-11-((3-hydroxy-3-methylcyclobutyl)ethynyl)-6-(methyl-d3)-6,7-dihydro-7,14-methanobenzo[f]benzo[4,5]imidazo[1,2-a][1,4]diazocin-5(14H)-one

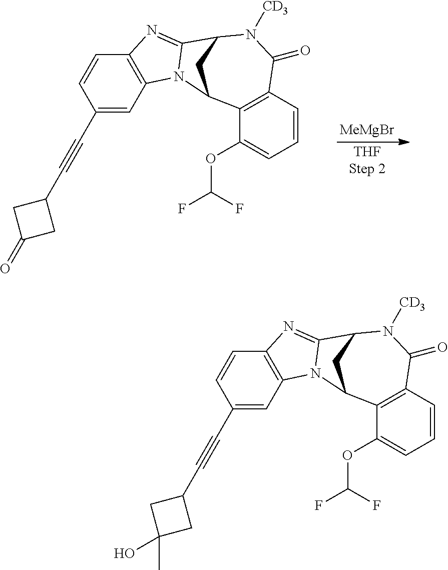 [SMILES: "MeMgBr" ; [2H]C([2H])([2H])N1C(=O)c2cccc(OC(F)F)c2[C@H]2C[C@@H]1c1nc3ccc(C#CC4CC(=O)C4)cc3n12 ; "THFStep 2" ; [2H]C([2H])([2H])N1C(=O)c2cccc(OC(F)F)c2[C@H]2C[C@@H]1c1nc3ccc(C#CC4CC(C)(O)C4)cc3n12]

To a stirred mixture of (7R,14R)-1-(difluoromethoxy)-6-(methyl-d3)-11-((3-oxocyclobutyl)ethynyl)-6,7-dihydro-7,14-methanobenzo[f]benzo[4,5]imidazo[1,2-a][1,4]diazocin-5(14H)-one (37 mg, 0.082 mmol) in THF (1 mL) was added 3 M CH₃MgBr (33 uL, 0.098 mmol) at 0° C. under nitrogen atmosphere. The resulting mixture was stirred for 4 h at room temperature. The reaction was quenched with Water at room temperature. The resulting mixture was concentrated under vacuum and purified by reversed-phase flash chromatography with the following conditions: column, C18 silica gel; mobile phase, CH₃CN in Water (10 mmol/L NH₄HCO₃), 25% to 55% gradient in 25 min; detector, 254 nm. This resulted in (7R,14R)-1-(difluoromethoxy)-11-((3-hydroxy-3-methylcyclobutyl)ethynyl)-6-(methyl-d3)-6,7-dihydro-7,14-methanobenzo[f]benzo[4,5]imidazo[1,2-a][1,4]diazocin-5(14H)-one (2 mg, 5%) as a white solid. MS ESI calculated for C₂₆H₂₀D₃F₂N₃O₃[M+H]⁺, 467.19. found 467.25. ¹H NMR (400 MHz, Chloroform-d) δ 8.51-8.47 (m, 1H), 7.63 (d, J=8.4 Hz, 1H), 7.53-7.48 (m, 1H), 7.46-7.40 (m, 1H), 7.39-7.34 (m, 1H), 7.30 (d, J=8.1 Hz, 1H), 7.04-6.65 (m, 1H), 6.23 (d, J=6.9 Hz, 1H), 5.10-4.98 (m, 1H), 3.52-3.40 (m, 1H), 2.88 (d, J=13.3 Hz, 1H), 2.84-2.73 (m, 1H), 2.56-2.46 (m, 2H), 2.37-2.26 (m, 2H), 1.40 (s, 3H). ¹⁹F NMR (377 MHz, Chloroform-d) δ −79.97, −80.42, −81.18, −81.62.

### Example 178: (7R,14R)-1-(difluoromethoxy)-6-(methyl-d3)-11-((3-methyl-1-(2,2,2-trifluoroethyl)azetidin-3-yl)ethynyl)-6,7-dihydro-7,14-methanobenzo[f]benzo[4,5]imidazo[1,2-a][1,4]diazocin-5(14H)-one

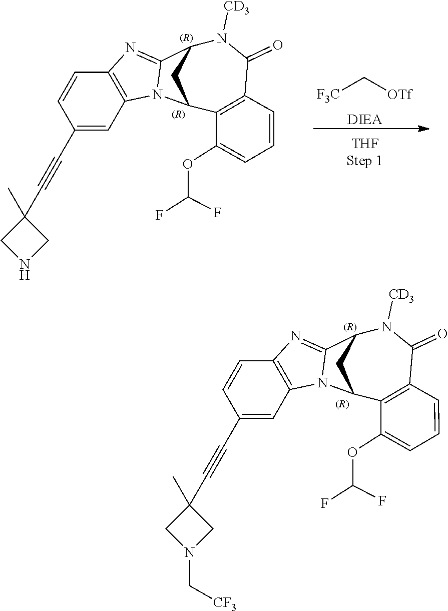

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (28 mg, 0.062 mmol) and DIEA (16 mg, 0.124 mmol) in THF (0.5 mL) was added 2,2,2-trifluoroethyl trifluoromethanesulfonate (22 mg, 0.093 mmol). The solution was stirred for 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in (7R,14R)-1-(difluoromethoxy)-6-(methyl-d3)-11-((3-methyl-1-(2,2,2-trifluoroethyl)azetidin-3-yl)ethynyl)-6,7-dihydro-7,14-methanobenzo[f]benzo[4,5]imidazo[1,2-a][1,4]diazocin-5(14H)-one (18 mg, 56%) as a white solid. MS ESI calculated for C₂₇H₂₀D₃F₅N₄O₂[M+H]⁺, 534.19. found 534.15. ¹H NMR (400 MHz, Chloroform-d) δ 8.53-8.37 (m, 1H), 7.62 (d, J=8.4 Hz, 1H), 7.50 (s, 1H), 7.42 (t, J=8.2 Hz, 1H), 7.37-7.27 (m, 2H), 6.85 (t, J=72.7 Hz, 1H), 6.21 (d, J=7.2 Hz, 1H), 4.95 (d, J=7.1 Hz, 1H), 3.59-3.49 (m, 4H), 3.48-3.40 (m, 1H), 3.07 (q, J=9.3 Hz, 2H), 2.86 (d, J=13.5 Hz, 1H), 1.68 (s, 3H). ¹⁹F NMR (377 MHz, Chloroform-d) δ −70.83, −80.00, −80.44, −81.17, −81.62.

### Example 179: (7R,14R)-1-(difluoromethoxy)-11-((R)-3-hydroxybut-1-yn-1-yl)-6-(methyl-d3)-6,7-dihydro-7,14-methanobenzo[f]benzo[4,5]imidazo[1,2-a][1,4]diazocin-5(14H)-one

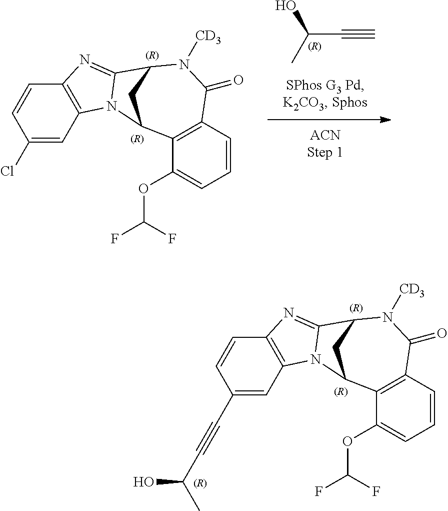

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.102 mmol), SPhos (8 mg, 0.020 mmol), K₂CO₃ (70 mg, 0.510 mmol) and SPhos Pd Gen.3 (15 mg, 0.020 mmol) in ACN (1 mL) was added (2R)-but-3-yn-2-ol (35 mg, 0.510 mmol) at room temperature under nitrogen atmosphere. The resulting mixture was stirred for 16 h at 80° C. under nitrogen atmosphere. The resulting mixture was concentrated under reduced pressure and purified by silica gel column chromatography, eluted with CH₂Cl₂/MeOH (0% to 10%) followed by Prep-HPLC with the following conditions (Column: XBridge Shield RP18 OBD Column 30*150 mm, 5m; Mobile Phase A: Water (10 mmol/L NH₄HCO₃), Mobile Phase B: ACN; Flow rate: 60 mL/min mL/min; Gradient: 23% B to 43% B in 10 min; Wave Length: 254 nm; RT1(min):9.55) to afford (7R,14R)-1-(difluoromethoxy)-11-((R)-3-hydroxybut-1-yn-1-yl)-6-(methyl-d3)-6,7-dihydro-7,14-methanobenzo[f]benzo[4,5]imidazo[1,2-a][1,4]diazocin-5(14H)-one (12 mg, 26%) as a white solid. MS ESI calculated for C₂₃H₁₆D₃F₂N₃O₃ [M+H]⁺, 427.16. found 427.00. ¹H NMR (400 MHz, Chloroform-d) δ 8.57-8.47 (m, 1H), 7.65 (d, J=8.5 Hz, 1H), 7.54 (s, 1H), 7.44 (t, J=8.2 Hz, 1H), 7.39-7.29 (m, 2H), 6.85 (t, J=72.2 Hz, 1H), 6.24 (d, J=7.2 Hz, 1H), 5.05 (d, J=7.0 Hz, 1H), 4.78 (q, J=6.6 Hz, 1H), 3.55-3.40 (m, 1H), 2.88 (d, J=13.6 Hz, 1H), 1.57 (d, J=6.6 Hz, 3H). ¹⁹F NMR (377 MHz, Chloroform-d) δ −80.05, −80.50, −81.18, −81.63.

### Example 180: (7R,14R)-11-((1-acetyl-4-methylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

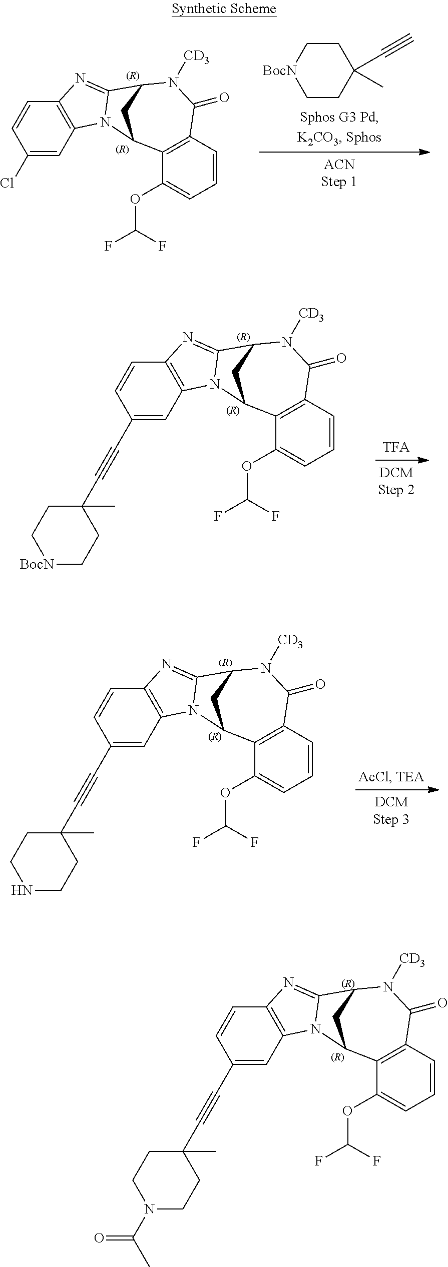

### Preparation 180A: tert-butyl 4-(((7(714R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-4-methylpiperidine-1-carboxylate

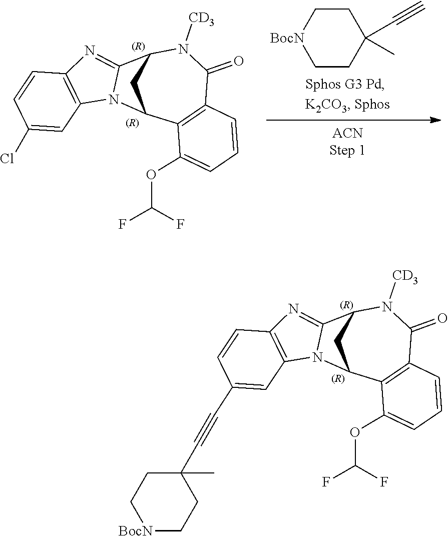

To a solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (600 mg, 1.527 mmol) and tert-butyl 4-ethynyl-4-methylpiperidine-1-carboxylate (512 mg, 2.290 mmol) in ACN (25 mL) were added K₂CO₃ (1.06 g, 7.635 mmol), SPhos (125 mg, 0.305 mmol) and SPhos Pd Gen.3 (238 mg, 0.305 mmol) at room temperature. The resulting mixture was stirred for 4 h at 80° C. under nitrogen atmosphere. The reaction was quenched by the addition of Water (100 mL) at room temperature. The resulting mixture was extracted with EtOAc (3×50 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 40% to 55% gradient in 20 min; detector, 254 nm to afford tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-4-methylpiperidine-1-carboxylate (800 mg, 90%) as a white solid. MS ESI calculated for C₃₂H₃₁D₃F₂N₄O₄ [M+H]⁺, 580.27 found 580.25. ¹H NMR (400 MHz, Chloroform-d) δ 8.52-8.42 (m, 1H), 7.60 (d, J=8.4 Hz, 1H), 7.51 (d, J=1.5 Hz, 1H), 7.43 (d, J=8.2 Hz, 1H), 7.32 (d, J=8.1 Hz, 1H), 7.29-7.24 (m, 1H), 6.85 (t, J=72.8 Hz, 1H), 6.20 (d, J=7.2 Hz, 1H), 4.93 (d, J=7.1 Hz, 1H), 4.15-3.90 (m, 2H), 3.48-3.37 (m, 1H), 3.24-3.08 (m, 2H), 2.85 (d, J=13.6 Hz, 1H), 1.79 (d, J=14.5 Hz, 2H), 1.51-1.37 (m, 11H), 1.34 (s, 3H). ¹⁹F NMR (377 MHz, Chloroform-d) δ −80.14, −80.59, −81.32, −81.76.

### Preparation 180B: (7R,14R)-1-(difluoromethoxy)-6-(methyl-d3)-11-((4-methylpiperidin-4-yl)ethynyl)-6,7-dihydro-7,14-methanobenzo[f]benzo[4,5]imidazo[1,2-a][1,4]diazocin-5(14H)-one

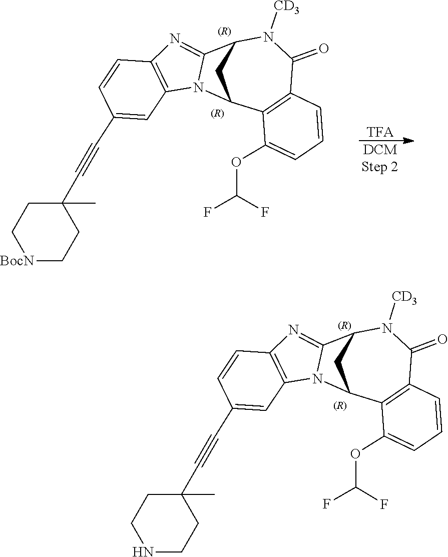

To a solution of tert-butyl 4-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-4-methylpiperidine-1-carboxylate (800 mg, 1.380 mmol) in DCM (25 mL) was added TFA (5 mL). The reaction was stirred for 1 h at room temperature. The resulting mixture was concentrated under reduced pressure. The mixture was neutralized to pH 7 with saturated NaHCO₃(aq.). The resulting mixture was extracted with EtOAc (3×50 mL). The combined organic layers were concentrated under reduced pressure. This resulted in (7R,14R)-1-(difluoromethoxy)-6-(methyl-d3)-11-((4-methylpiperidin-4-yl)ethynyl)-6,7-dihydro-7,14-methanobenzo[f]benzo[4,5]imidazo[1,2-a][1,4]diazocin-5(14H)-one (1 g, crude) as a yellow solid, which was used in the next step directly without further purification. MS ESI calculated for C₂₇H₂₃D₃F₂N₄O₂[M+H]⁺, 480.22. found 480.25. ¹H NMR (400 MHz, Chloroform-d) δ 8.51-8.43 (m, 1H), 7.61 (d, J=8.4 Hz, 1H), 7.53 (d, J=1.5 Hz, 1H), 7.43 (d, J=8.2 Hz, 1H), 7.33 (d, J=8.2 Hz, 1H), 7.28 (d, J=1.6 Hz, 1H), 7.10-6.70 (m, 1H), 6.21 (d, J=7.2 Hz, 1H), 4.93 (d, J=7.1 Hz, 1H), 3.51-3.38 (m, 1H), 3.18-3.01 (m, 4H), 2.85 (d, J=13.6 Hz, 1H), 1.83 (d, J=13.3 Hz, 2H), 1.63-1.45 (m, 2H), 1.35 (s, 3H). ¹⁹F NMR (377 MHz, Chloroform-d) δ −80.18, −80.62, −81.46, −81.91.

### Example 180: (7R,14R)-11-((1-acetyl-4-methylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

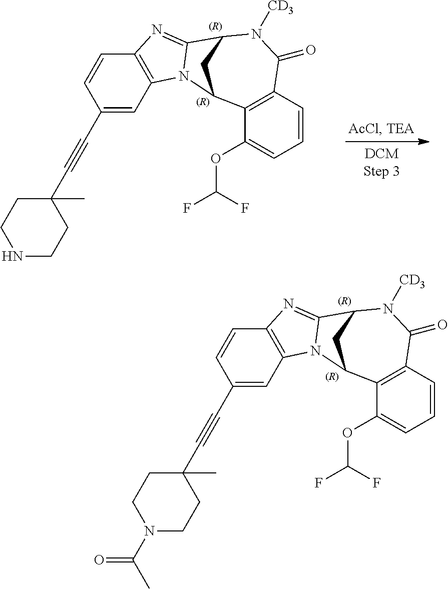

To a solution of (7R,14R)-1-(difluoromethoxy)-6-(methyl-d3)-11-((4-methylpiperidin-4-yl)ethynyl)-6,7-dihydro-7,14-methanobenzo[f]benzo[4,5]imidazo[1,2-a][1,4]diazocin-5(14H)-one (66 mg, 0.138 mmol) and TEA (35 mg, 0.345 mmol) in DCM (10 mL) was added acetyl chloride (12 mg, 0.152 mmol) at room temperature. The resulting mixture was stirred for additional 1 h at room temperature. The resulting mixture was concentrated under reduced pressure and purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 26% B to 41% B in 10 min; Wave Length: 254 nm; RT1(min): 11.90) to afford (7R,14R)-11-((1-acetyl-4-methylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (42 mg, 59%) as a white solid. MS ESI calculated for C₂₉H₂₅D₃F₂N₄O₃[M+H]⁺, 522.23. found 522.30. ¹H NMR (400 MHz, Chloroform-d) δ 8.54-8.45 (m, 1H), 7.67-7.61 (m, 1H), 7.54 (d, J=1.5 Hz, 1H), 7.43 (t, J=8.2 Hz, 1H), 7.35-7.27 (m, 2H), 6.85 (t, J=72.9 Hz, 1H), 6.22 (d, J=7.1 Hz, 1H), 4.96 (d, J=7.0 Hz, 1H), 4.56 (d, J=13.3 Hz, 1H), 3.73 (d, J=13.7 Hz, 1H), 3.57-3.36 (m, 2H), 3.10-2.95 (m, 1H), 2.86 (d, J=13.5 Hz, 1H), 2.11 (s, 3H), 1.92-1.78 (m, 2H), 1.55-1.39 (m, 2H), 1.36 (s, 3H). ¹⁹F NMR (377 MHz, Chloroform-d) δ −80.34, −80.35, −80.78, −80.80, −81.07, −81.13, −81.52, −81.57.

### Example 181: (7R,14R)-11-((1-(cyclopropanecarbonyl)-4-methylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

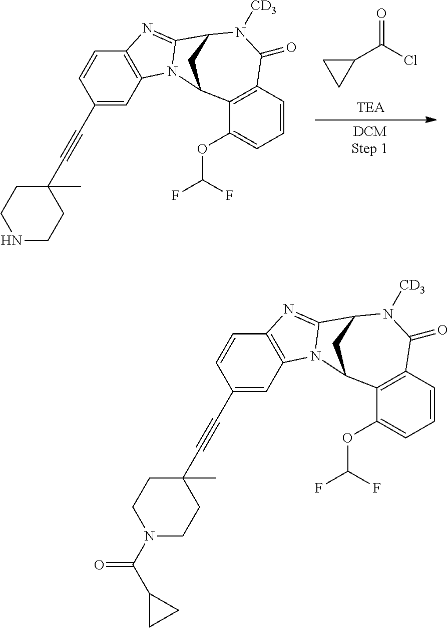

To a solution of (7R,14R)-1-(difluoromethoxy)-6-(methyl-d3)-11-((4-methylpiperidin-4-yl)ethynyl)-6,7-dihydro-7,14-methanobenzo[f]benzo[4,5]imidazo[1,2-a][1,4]diazocin-5(14H)-one (66 mg, 0.138 mmol) and TEA (35 mg, 0.345 mmol) in DCM (10 mL) was added cyclopropanecarbonyl chloride (16 mg, 0.152 mmol) at room temperature. The resulting mixture was stirred for additional 1 h at room temperature. The resulting mixture was concentrated under reduced pressure and purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 31% B to 46% B in 10 min; Wave Length: 254 nm; RT1(min): 11.73) to afford (7R,14R)-11-((1-(cyclopropanecarbonyl)-4-methylpiperidin-4-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (43 mg, 57%) as a white solid. MS ESI calculated for C₃₁H₂₇D₃F₂N₄O₃[M+H]⁺, 548.25 found 538.30. ¹H NMR (400 MHz, Chloroform-d) δ 8.55-8.43 (m, 1H), 7.68-7.60 (m, 1H), 7.57-7.53 (m, 1H), 7.43 (t, J=8.2 Hz, 1H), 7.35-7.28 (m, 2H), 6.86 (t, J=72.8 Hz, 1H), 6.23 (d, J=7.2 Hz, 1H), 4.99 (d, J=7.1 Hz, 1H), 4.55 (d, J=13.5 Hz, 1H), 4.14 (d, J=13.7 Hz, 1H), 3.63-3.38 (m, 2H), 3.04 (t, J=13.0 Hz, 1H), 2.87 (d, J=13.6 Hz, 1H), 1.92-1.73 (m, 3H), 1.61-1.40 (m, 2H), 1.37 (s, 3H), 1.01-0.95 (m, 2H), 0.81-0.66 (m, 2H). ¹⁹F NMR (377 MHz, Chloroform-d) δ −80.32, −80.77, −81.17, −81.62.

### Example 182: (7R,14R)-1-(difluoromethoxy)-6-(methyl-d₃)-11-((3-methyl-1-(1-methylcyclopropane-1-carbonyl)pyrrolidin-3-yl)ethynyl)-6,7-dihydro-7,14-methanobenzo[f]benzo[4,5]imidazo[1,2-a][1,4]diazocin-5(14H)-one

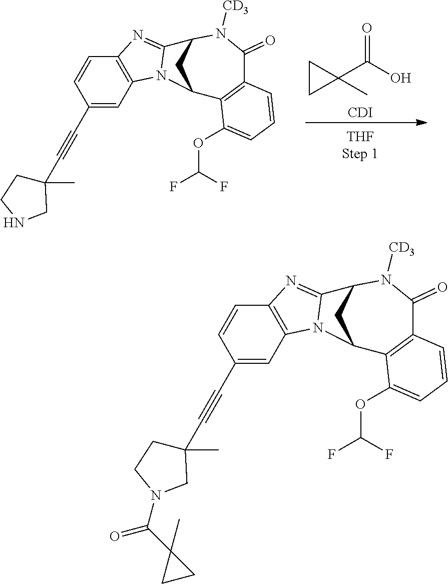

To a stirred solution of 1-methylcyclopropane-1-carboxylic acid (22 mg, 0.214 mmol) in THF (0.5 mL) was added CDI (35 mg, 0.214 mmol) at room temperature. The resulting mixture was stirred for 30 min at room temperature. To the above mixture was added (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5 (14H)-one (50 mg, 0.107 mmol) at room temperature. The resulting mixture was stirred for additional 1 h at room temperature. The resulting mixture was concentrated under reduced pressure and purified by reverse phase flash with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 29% B to 49% B in 10 min; Wave Length: 254 nm; RT1(min): 8.75;) to afford (7R,14R)-1-(difluoromethoxy)-6-(methyl-d₃)-11-((3-methyl-1-(1-methylcyclopropane-1-carbonyl)pyrrolidin-3-yl)ethynyl)-6,7-dihydro-7,14-methanobenzo[f]benzo[4,5]imidazo[1,2-a][1,4]diazocin-5(14H)-one (18 mg, 30%) as a white solid. MS ESI calculated for C₃₁H₂₇D₃F₂N₄O₃[M+H]⁺, 548.25. found 548.15. ¹H NMR (400 MHz, DMSO-d₆) δ 8.31-8.24 (m, 1H), 7.79-7.37 (m, 5H), 7.17 (d, J=8.2 Hz, 1H), 6.26-6.20 (m, 1H), 5.26-5.19 (m, 1H), 3.96-3.47 (m, 5H), 2.81 (d, J=13.8 Hz, 1H), 2.24-2.06 (m, 1H), 2.03-1.86 (m, 1H), 1.40 (s, 3H), 1.30-1.21 (m, 3H), 0.89-0.70 (m, 2H), 0.61-0.45 (m, 2H). ¹⁹F NMR (377 MHz, DMSO-d₆) δ −81.79, −82.22, −82.24, −82.27.

### Example 183: (7R,14R)-11-((1-acetylpiperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

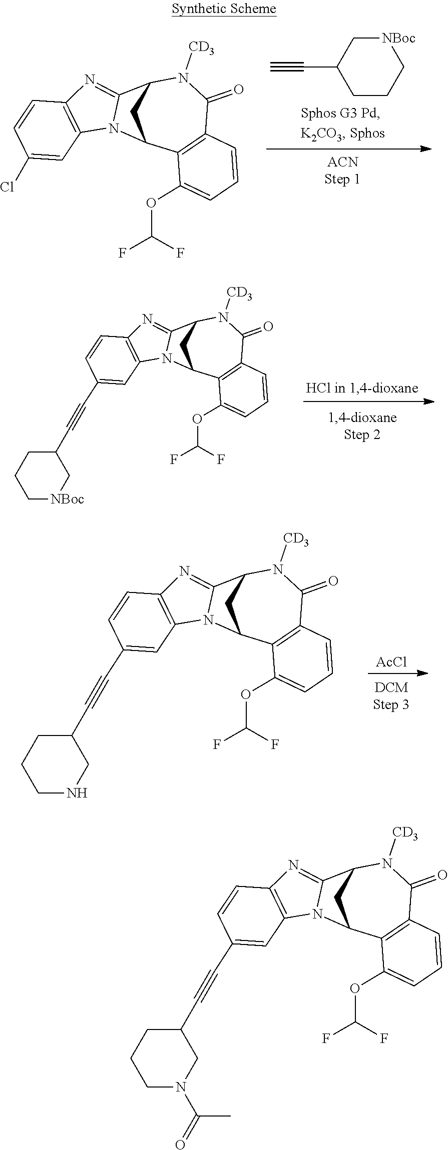

### Preparation 183A: tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)piperidine-1-carboxylate

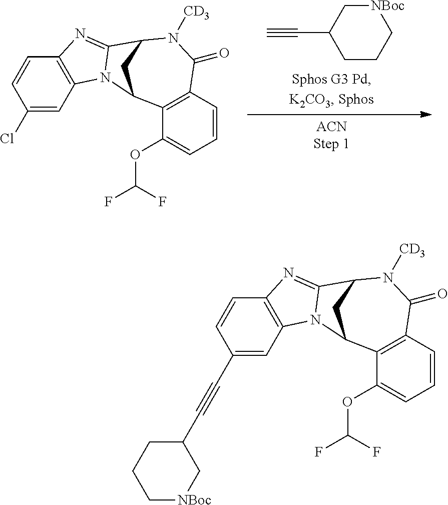

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (400 mg, 1.018 mmol) and tert-butyl 3-ethynylpiperidine-1-carboxylate (1.07 g, 5.090 mmol) in ACN (8 mL) were added K₂CO₃ (704 mg, 5.090 mmol), SPhos (84 mg, 0.204 mmol) and SPhos Pd Gen.3 (159 mg, 0.204 mmol) at room temperature. The resulting mixture was stirred for 1 h at 80° C. under nitrogen atmosphere. The resulting mixture was diluted with water (15 mL) and extracted with EtOAc (3×15 mL). The combined organic layers were washed with brine (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 10% to 95% gradient in 60 min; detector, 254 nm to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)piperidine-1-carboxylate (500 mg, 86%) as a yellow solid. MS ESI calculated for C₃₁H₂₉D₃F₂N₄O₄[M+H]⁺, 566.26 found 566.35. ¹H NMR (400 MHz, Chloroform-d) δ 8.51-8.46 (m, 1H), 7.64 (d, J=8.5 Hz, 1H), 7.53-7.50 (m, 1H), 7.43 (t, J=8.2 Hz, 1H), 7.38-7.29 (m, 2H), 7.09-6.87 (m, 1H), 6.28-6.20 (m, 1H), 5.06 (d, J=7.1 Hz, 1H), 4.21-3.59 (m, 4H), 3.55-3.40 (m, 1H), 2.88 (d, J=13.6 Hz, 1H), 2.71-2.63 (m, 1H), 2.10-1.99 (m, 1H), 1.76-1.61 (m, 3H), 1.46-1.33 (m, 9H).

### Preparation 183B: (7R,14R)-1-(difluoromethoxy)-6-(methyl-d₃)-11-(piperidin-3-ylethynyl)-6,7-dihydro-7,14-methanobenzo[f]benzo[4,5]imidazo[1,2-a][1,4]diazocin-5(14H)-one

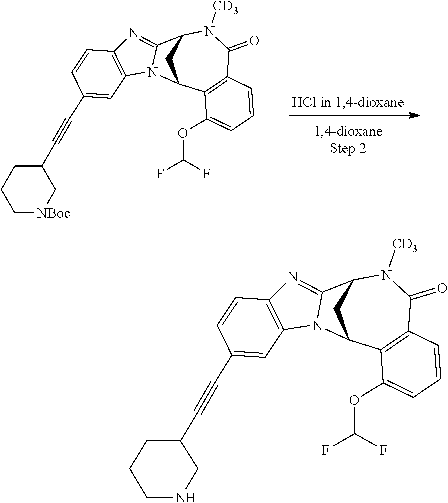 [SMILES: [2H]C([2H])([2H])N1C(=O)c2cccc(OC(F)F)c2[C@H]2C[C@@H]1c1nc3ccc(C#CC4CCCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CCCNC4)cc3n12]

To a stirred solution of tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)piperidine-1-carboxylate (495 mg, 0.875 mmol) in 1,4-dioxane (5.0 mL) was added 4 M HCl (gas) in 1,4-dioxane (5 mL) dropwise at room temperature. The resulting mixture was stirred for 1 h at room temperature. The resulting mixture was concentrated under reduced pressure. The resulting mixture was diluted with sat. NaHCO₃(aq.) (30 mL) extracted with EtOAc (3×10 mL). The combined organic layers were washed with brine (3×10 mL), dried over anhydrous Na₂SO₄. After filtration, the filtrate was concentrated under reduced pressure to afford (7R,14R)-1-(difluoromethoxy)-6-(methyl-d₃)-11-(piperidin-3-ylethynyl)-6,7-dihydro-7,14-methanobenzo[f]benzo[4,5]imidazo[1,2-a][1,4]diazocin-5(14H)-one (339 mg, crude) as a yellow solid. The crude product was used in the next step directly without further purification. MS ESI calculated for C₂₆H₂₁D₃F₂N₄O₂[M+H]⁺, 466.21. found 466.20.

### Example 183: (7R,14R)-11-((1-acetylpiperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

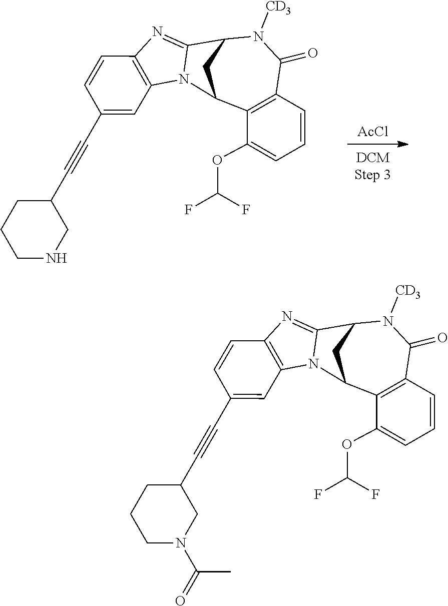 [SMILES: "AcCl" ; [2H]C([2H])([2H])N1C(=O)c2cccc(OC(F)F)c2[C@H]2C[C@@H]1c1nc3ccc(C#CC4CCCNC4)cc3n12 ; [2H]C([2H])([2H])N1C(=O)c2cccc(OC(F)F)c2[C@H]2C[C@@H]1c1nc3ccc(C#CC4CCCN(C(C)=O)C4)cc3n12]

To a stirred solution of (7R,14R)-1-(difluoromethoxy)-6-(methyl-d₃)-11-(piperidin-3-ylethynyl)-6,7-dihydro-7,14-methanobenzo[f]benzo[4,5]imidazo[1,2-a][1,4]diazocin-5(14H)-one (50 mg, 0.107 mmol) in DCM (0.5 mL) was added acetyl chloride (9 mg, 0.118 mmol) dropwise at room temperature under nitrogen atmosphere. The resulting mixture was stirred for 1 h at room temperature under nitrogen atmosphere. The resulting mixture was concentrated under reduced pressure and purified by reverse phase flash with the following conditions (Column: XBridge Shield RP18 OBD Column 30*150 mm, 5m; Mobile Phase A: Water (10 mmol/L NH₄HCO₃), Mobile Phase B: ACN; Flow rate: 60 m/min m/min; Gradient: 26% B to 46% B in 10 min; Wave Length: 254 nm; RT1(min): 9.5) to afford (7R,14R)-11-((1-acetylpiperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (21 mg, 38%) as a white solid. MS ESI calculated for C₂₈H₂₃D₃F₂N₄O₃[M+H]⁺, 508.22 found 508.20. ¹H NMR (400 MHz, DMSO-d₆) δ 8.31-8.24 (m, 1H), 7.80-7.38 (m, 5H), 7.20-7.15 (m, 1H), 6.25-6.19 (m, 1H), 5.25-5.18 (m, 1H), 4.14-3.44 (m, 4H), 3.26-2.99 (m, 1H), 2.91-2.61 (m, 2H), 2.09-1.88 (m, 4H), 1.78-1.58 (m, 2H), 1.53-1.36 (m, 1H). ¹⁹F NMR (282 MHz, DMSO-d₆) δ −81.95.

### Example 184: (7R,14R)-11-((1-(cyclopropanecarbonyl)piperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

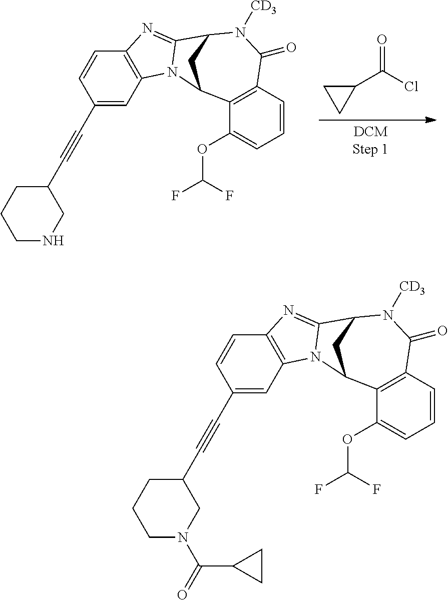

To a stirred solution of (7R,14R)-1-(difluoromethoxy)-6-(methyl-d₃)-11-(piperidin-3-ylethynyl)-6,7-dihydro-7,14-methanobenzo[f]benzo[4,5]imidazo[1,2-a][1,4]diazocin-5(14H)-one (50 mg, 0.107 mmol) in DCM (0.5 mL) was added cyclopropanecarbonyl chloride (12 mg, 0.118 mmol) at room temperature under nitrogen atmosphere. The resulting mixture was stirred for 1 h at room temperature. The resulting mixture was concentrated under reduced pressure and purified by reverse phase flash with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 m/min mL/min; Gradient: 35% B to 52% B in 10 min; Wave Length: 254 nm; RT1(min): 9.75) to afford (7R,14R)-11-((1-(cyclopropanecarbonyl)piperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (21 mg, 36%) as a white solid. MS ESI calculated for C₃₀H₂₅D₃F₂N₄O₃[M+H]⁺, 534.23. found 534.20. ¹H NMR (400 MHz, DMSO-d₆) δ 8.35-8.21 (m, 1H), 7.81-7.35 (m, 5H), 7.26-7.15 (m, 1H), 6.22 (d, J=7.1 Hz, 1H), 5.22 (d, J=7.1 Hz, 1H), 4.17-3.67 (m, 2H), 3.57-3.34 (m, 3H), 2.96-2.60 (m, 2H), 2.09-1.89 (m, 2H), 1.84-1.64 (m, 2H), 1.56-1.33 (m, 1H), 0.78-0.48 (m, 4H). ¹⁹F NMR (282 MHz, DMSO-d₄) δ −81.92, −81.96, −82.00, −82.08.

### Example 185: (7R,14R)-1-(difluoromethoxy)-6-(methyl-d3)-11-((1-(methylsulfonyl)piperidin-4-yl)ethynyl)-6,7-dihydro-7,14-methanobenzo[f]benzo[4,5]imidazo[1,2-a][1,4]diazocin-5(14H)-one

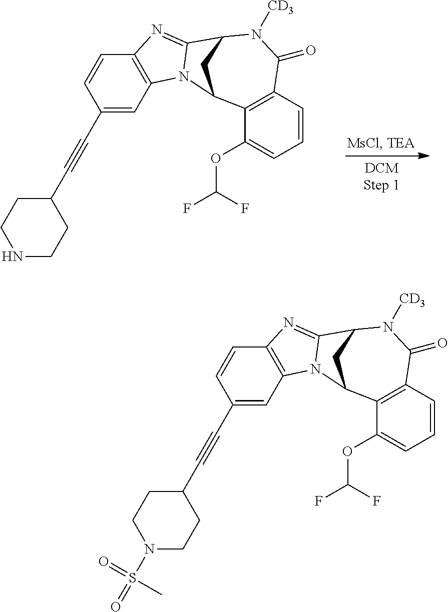 [SMILES: [2H]C([2H])([2H])N1C(=O)c2cccc(OC(F)F)c2[C@H]2C[C@@H]1c1nc3ccc(C#CC4CCNCC4)cc3n12 ; "MsCl, TEA" ; "DCMStep 1" ; [2H]C([2H])([2H])N1C(=O)c2cccc(OC(F)F)c2[C@H]2C[C@@H]1c1nc3ccc(C#CC4CCN(S(C)(=O)=O)CC4)cc3n12]

To a solution of (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (50 mg, 0.107 mmol) and TEA (55 mg, 0.535 mmol) in DCM (8 mL) was added methanesulfonyl chloride (25 mg, 0.214 mmol) at room temperature. The resulting mixture was stirred for additional 1 h at room temperature. The resulting mixture was concentrated under reduced pressure and purified by Prep-HPLC with the following conditions (Column: XBridge Shield RP18 OBD Column 30*150 mm, 5m; Mobile Phase A: EtOH (0.1% NH₃·H₂O), Mobile Phase B: ACN; Flow rate: 60 mL/min; Gradient: 27% B to 47% B in 10 min; Wave Length: 254 nm; RT1(min): 10.98) to afford (7R,14R)-1-(difluoromethoxy)-6-(methyl-d3)-11-((1-(methylsulfonyl)piperidin-4-yl)ethynyl)-6,7-dihydro-7,14-methanobenzo[f]benzo[4,5]imidazo[1,2-a][1,4]diazocin-5(14H)-one (38 mg, 64%) as a white solid. MS ESI calculated for C₂₇H₂₃D₃F₂N₄O₄S [M+H]⁺, 544.18 found 544.20. ¹H NMR (400 MHz, Chloroform-d) δ 8.52-8.45 (m, 1H), 7.65 (d, J=8.3 Hz, 1H), 7.54 (s, 1H), 7.44 (t, J=8.2 Hz, 1H), 7.36-7.29 (m, 2H), 6.85 (t, J=72.8 Hz, 1H), 6.29-6.17 (m, 1H), 5.15-4.90 (m, 1H), 3.57-3.39 (m, 3H), 3.33-3.21 (m, 2H), 3.01-2.87 (m, 2H), 2.80 (s, 3H), 2.13-1.91 (m, 4H). ¹⁹F NMR (377 MHz, Chloroform-d) δ −80.22, −80.67, −80.73, −81.18.

### Example 186: (7R,14R)-1-(difluoromethoxy)-11-((1-(2-hydroxy-2-methylpropanoyl)piperidin-4-yl)ethynyl)-6-(methyl-d3)-6,7-dihydro-7,14-methanobenzo[f]benzo[4,5]imidazo[1,2-a][1,4]diazocin-5(14H)-one

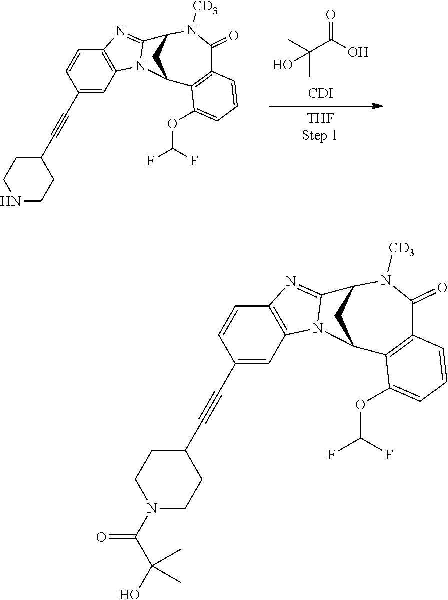

A solution of 2-hydroxy-2-methylpropanoic acid (89 mg, 0.860 mmol) and CDI (139 mg, 0.860 mmol) in THF (20 mL) was stirred for 10 min at room temperature. To the above mixture was added (7R,14R)-1-(difluoromethoxy)-6-(methyl-d3)-11-(piperidin-4-ylethynyl)-6,7-dihydro-7,14-methanobenzo[f]benzo[4,5]imidazo[1,2-a][1,4]diazocin-5(14H)-one (100 mg, 0.215 mmol) at room temperature. The resulting mixture was stirred for additional 2 h at room temperature. The resulting mixture was concentrated under reduced pressure and purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 23% B to 38% B in 10 min; Wave Length: 254 nm; RT1(min): 13.35) to afford (7R,14R)-1-(difluoromethoxy)-11-((1-(2-hydroxy-2-methylpropanoyl)piperidin-4-yl)ethynyl)-6-(methyl-d3)-6,7-dihydro-7,14-methanobenzo[f]benzo[4,5]imidazo[1,2-a][1,4]diazocin-5(14H)-one (42 mg, 35%) as a white solid. MS ESI calculated for C₃₀H₂₇D₃F₂N₄O₄[M+H]⁺, 551.24 found 552.30. ¹H NMR (400 MHz, Chloroform-d) δ 8.52-8.45 (m, 1H), 7.64 (d, J=8.4 Hz, 1H), 7.53 (s, 1H), 7.43 (t, J=8.0 Hz, 1H), 7.36-7.26 (m, 2H), 6.84 (t, J=72.8 Hz, 1H), 6.22 (d, J=7.2 Hz, 1H), 4.99 (d, J=7.2 Hz, 1H), 4.61 (s, 1H), 3.99-3.89 (m, 2H), 3.64-3.53 (m, 2H), 3.51-3.40 (m, 1H), 3.02-2.92 (m, 1H), 2.87 (d, J=13.6 Hz, 1H), 2.01-1.93 (m, 2H), 1.82-1.74 (m, 2H), 1.52 (s, 6H). ¹⁹F NMR (377 MHz, Chloroform-d) δ −80.18, −80.63, −80.90, −81.34.

### Example 187: (7R,14R)-1-(difluoromethoxy)-11-((1-(1-hydroxycyclopropane-1-carbonyl)piperidin-4-yl)ethynyl)-6-(methyl-d3)-6,7-dihydro-7,14-methanobenzo[f]benzo[4,5]imidazo[1,2-a][1,4]diazocin-5(14H)-one

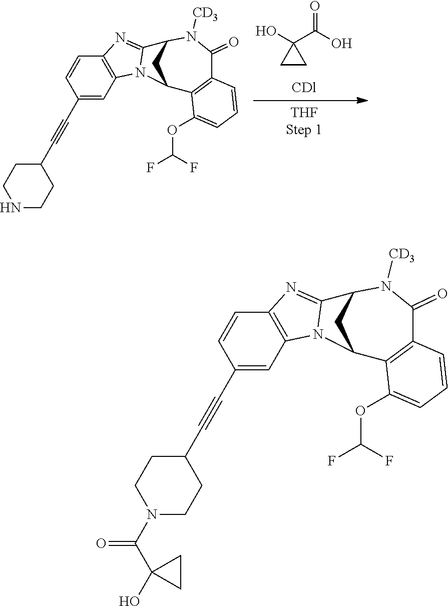

A solution of 1-hydroxycyclopropane-1-carboxylic acid (44 mg, 0.428 mmol) and CDI (70 mg, 0.428 mmol) in THF (10 mL) was stirred for 10 min at room temperature. To the above mixture was added (7R,14R)-1-(difluoromethoxy)-11-((1-(1-hydroxycyclopropane-1-carbonyl)piperidin-4-yl)ethynyl)-6-(methyl-d3)-6,7-dihydro-7,14-methanobenzo[f]benzo[4,5]imidazo[1,2-a][1,4]diazocin-5(14H)-one (50 mg, 0.107 mmol) at room temperature. The resulting mixture was stirred for additional 2 h at room temperature. The resulting mixture was concentrated under reduced pressure and purified by Prep-HPLC with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 23% B to 38% B in 10 min; Wave Length: 254 nm; RT1(min): 13.35) to afford (7R,14R)-1-(difluoromethoxy)-11-((1-(1-hydroxycyclopropane-1-carbonyl)piperidin-4-yl)ethynyl)-6-(methyl-d3)-6,7-dihydro-7,14-methanobenzo[f]benzo[4,5]imidazo[1,2-a][1,4]diazocin-5(14H)-one (41 mg, 67%) as a white solid. MS ESI calculated for C₃₀H₂₅D₃F₂N₄O₄ [M+H]⁺, 550.23 found 550.30. ¹H NMR (400 MHz, Chloroform-d) δ 8.50-8.42 (m, 1H), 7.62 (d, J=8.5 Hz, 1H), 7.55-7.50 (m, 1H), 7.43 (t, J=8.2 Hz, 1H), 7.36-7.28 (m, 2H), 6.84 (t, J=72.7 Hz, 1H), 6.23 (d, J=7.2 Hz, 1H), 5.04 (d, J=7.1 Hz, 1H), 4.10-3.93 (m, 2H), 3.62-3.40 (m, 3H), 3.02-2.76 (m, 2H), 2.14-1.84 (m, 4H), 1.80-1.64 (m, 2H), 1.21-1.04 (m, 2H). ¹⁹F NMR (377 MHz, Chloroform-d) δ −80.11, −80.56, −81.06, −81.51.

### Example 188: (7R,14R)-1-(difluoromethoxy)-11-((1-(difluoromethyl)cyclopropyl)ethynyl)-6-(methyl-d3)-6,7-dihydro-7,14-methanobenzo[f]benzo[4,5]imidazo[1,2-a][1,4]diazocin-5(14H)-one

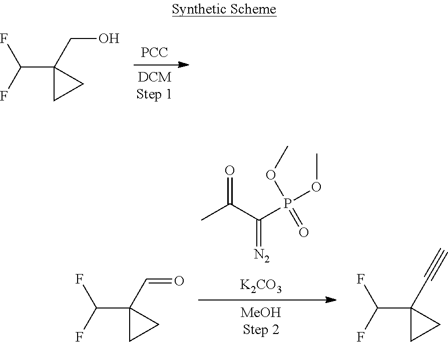

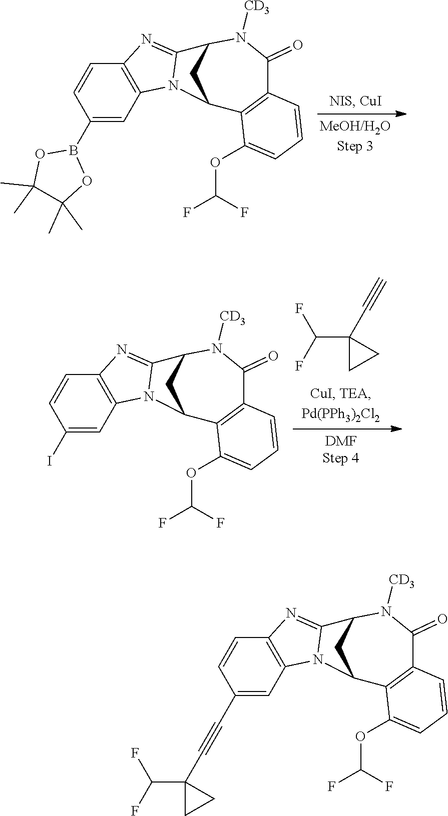

### Preparation 188A: 1-(difluoromethyl)cyclopropane-1-carbaldehyde

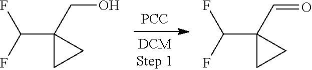

To a stirred solution of [1-(difluoromethyl)cyclopropyl]methanol (500 mg, 4.095 mmol) in DCM (5 mL) was added PCC (1.32 g, 6.143 mmol) in portions at room temperature. The mixture was stirred at 25° C. for 16 h. The reaction mixture was diluted with diethyl ether, the solvent was decanted and concentrated at 0° C. to afford the crude 1-(difluoromethyl)cyclopropane-1-carbaldehyde (500 mg) as a light yellow oil. MS ESI calculated for C₅H₆F₂O [M+H]⁺, 121.04. found N/A. ¹H NMR (400 MHz, Chloroform-d) δ 9.11 (t, J=2.4 Hz, 1H), 6.29 (t, J=56.4 Hz, 1H), 1.45-1.38 (m, 2H), 1.36-1.30 (m, 2H). ¹⁹F NMR (377 MHz, Chloroform-d) δ −122.33.

### Preparation 188B: 1-(difluoromethyl)-1-ethynylcyclopropane

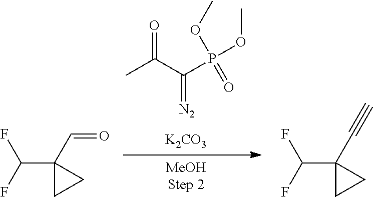

To a solution of [1-(difluoromethyl)cyclopropyl]methanol (500 mg, 4.095 mmol) and K₂CO₃ (1.15 g, 8.326 mmol) in MeOH (7 mL) was added dimethyl(1-diazo-2-oxopropyl)phosphonate (1.20 g, 6.245 mmol). The mixture was stirred at 25° C. for 2 h. The mixture was diluted with water (40 mL) and extracted with diethyl ether (3×20 mL). The combined organic layer was washed with brine (50 mL) and dried over sodium sulfate, filtered. The organic layer was concentrated to remove petroleum ether to give the crude 1-(difluoromethyl)-1-ethynylcyclopropane (400 mg) as colorless oil. MS ESI calculated for C₆H₆F₂[M+H]⁺, 117.04, found N/A. ¹H NMR (400 MHz, Chloroform-d) δ 5.83-5.51 (m, 1H), 2.10-2.07 (m, 1H), 1.15-1.03 (m, 4H). ¹⁹F NMR (377 MHz, Chloroform-d) δ −118.95.

### Preparation 188C: (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one

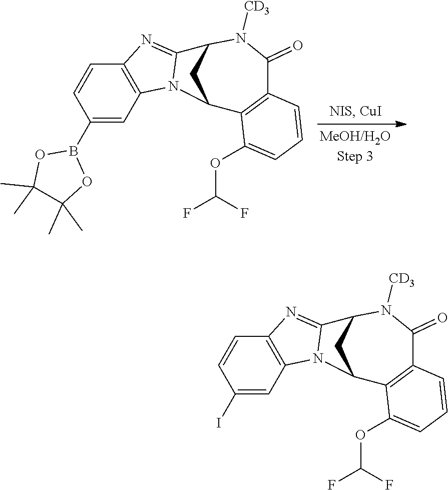 [SMILES: [2H]C([2H])([2H])N1C(=O)c2cccc(OC(F)F)c2[C@H]2C[C@@H]1c1nc3ccc(B4OC(C)(C)C(C)(C)O4)cc3n12 ; [2H]C([2H])([2H])N1C(=O)c2cccc(OC(F)F)c2[C@H]2C[C@@H]1c1nc3ccc(I)cc3n12]

To a stirred mixture of (7R,14R)-1-(difluoromethoxy)-6-(methyl-d3)-11-(4,4,5,5-tetramethyl-1,3,2-dioxaborolan-2-yl)-6,7-dihydro-7,14-methanobenzo[f]benzo[4,5]imidazo[1,2-a][1,4]diazocin-5(14H)-one (10.00 g, 20.647 mmol), CuI (5.90 g, 30.970 mmol) in MeOH (200 mL) and H₂O (20 mL) was added NIS (6.97 g, 30.970 mmol) at 0° C. The resulting mixture was stirred for 1 h at 80° C. The reaction was quenched with sat. NaHCO₃(aq.) at room temperature. The resulting mixture was filtered, the filter cake was washed with DCM (3×50 mL). The filtrate was concentrated under reduced pressure. The resulting mixture was extracted with DCM (3×300 mL), dried over anhydrous Na₂SO₄. After filtration, the filtrate was concentrated under reduced pressure. The residue was purified by silica gel column chromatography, eluted with DCM/MeOH (9/1) followed purified by trituration with PE (30 mL) to afford (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (9.20 g, 92%) as a yellow solid. MS ESI calculated for C₁₉H₁₁D₃F₂IN₃O₂[M+H]⁺, 485.03. found 485.00. ¹H NMR (400 MHz, Chloroform-d) δ 8.55-8.41 (m, 1H), 7.81 (d, J=1.6 Hz, 1H), 7.55-7.49 (m, 1H), 7.49-7.39 (m, 2H), 7.34 (d, J=8.2 Hz, 1H), 7.06-6.59 (m, 1H), 6.19 (d, J=7.0 Hz, 1H), 4.94 (d, J=6.8 Hz, 1H), 3.55-3.33 (m, 1H), 2.86 (d, J=13.4 Hz, 1H). ¹⁹F NMR (377 MHz, Chloroform-d) δ −80.05, −80.49, −81.23, −81.68.

### Example 188: (7R,14R)-1-(difluoromethoxy)-11-((1-(difluoromethyl)cyclopropyl)ethynyl)-6-(methyl-d3)-6,7-dihydro-7,14-methanobenzo[f]benzo[4,5]imidazo[1,2-a][1,4]diazocin-5(14H)-one

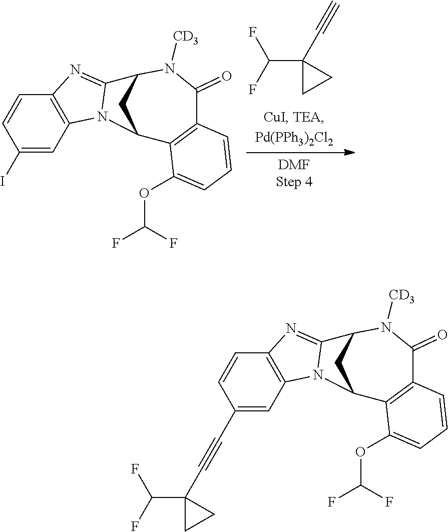

To a solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (70 mg, 0.145 mmol) and 1-(difluoromethyl)-1-ethynylcyclopropane (280 mg, 0.595 mmol) in TEA (0.5 mL) and DMF (0.5 mL) were added CuI (2 mg, 0.014 mmol) and Pd(PPh₃)₂Cl₂ (10 mg, 0.014 mmol). After stirring for 1 h at 80° C. under a nitrogen atmosphere. The mixture was purified by reversed-phase flash chromatography with the following conditions: column, C18 silica gel; mobile phase, CH₃CN in Water (10 mmol/L NH₄HCO₃), 35% to 75% gradient in 95 min; detector, 254 nm to afford (7R,14R)-1-(difluoromethoxy)-11-((1-(difluoromethyl)cyclopropyl)ethynyl)-6-(methyl-d3)-6,7-dihydro-7,14-methanobenzo[f]benzo[4,5]imidazo[1,2-a][1,4]diazocin-5(14H)-one (34 mg, 50%) as a white solid. MS ESI calculated for C₂₅H₁₆D₃F₄N₃O₂ [M+H]⁺, 473.16. found 473.20. ¹H NMR (400 MHz, Chloroform-d) δ 8.52-8.43 (m, 1H), 7.65 (d, J=8.5 Hz, 1H), 7.55 (s, 1H), 7.45 (t, J=8.2 Hz, 1H), 7.35 (t, J=7.5 Hz, 2H), 6.85 (t, J=72.6 Hz, 1H), 6.26 (d, J=7.2 Hz, 1H), 5.79 (t, J=57.6 Hz, 1H), 5.09 (d, J=7.1 Hz, 1H), 3.56-3.43 (m, 1H), 2.89 (d, J=13.6 Hz, 1H), 1.29-1.12 (m, 4H). ¹⁹F NMR (377 MHz, Chloroform-d) δ −80.12, −80.57, −81.39, −81.83, −118.84.

### Example 189: (7R,14R)-11-((1-(cyclopropanecarbonyl)-3-methylpyrrolidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)one

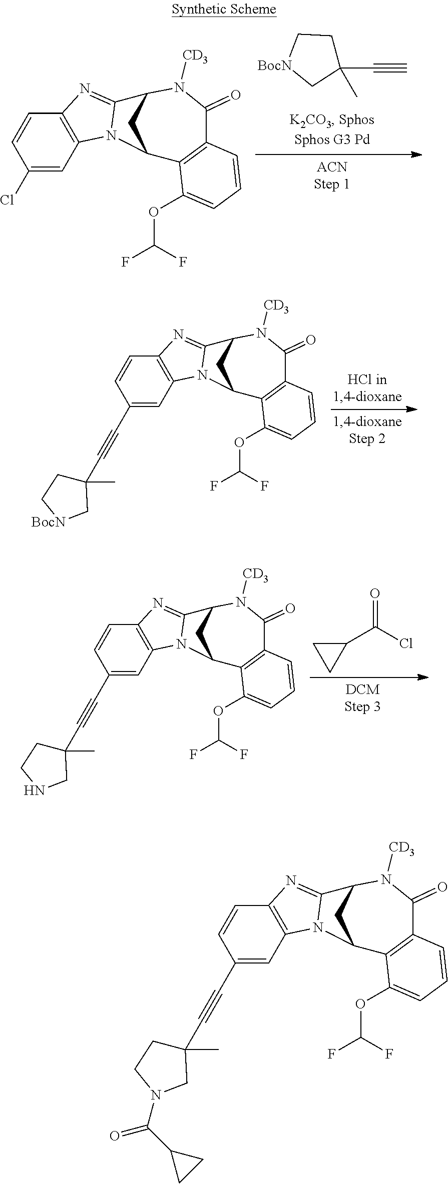

### Preparation 189A: tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)-3-methylpyrrolidine-1-carboxylate

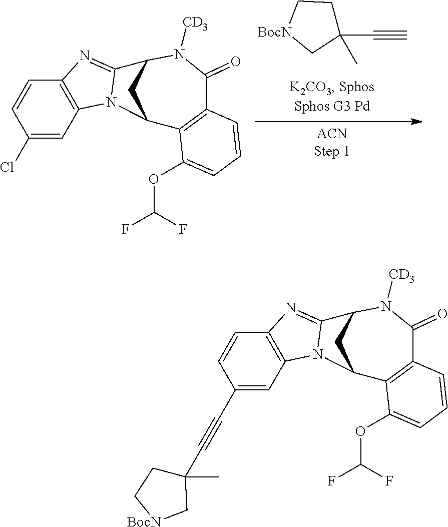 [SMILES: [2H]C([2H])([2H])N1C(=O)c2cccc(OC(F)F)c2[C@H]2C[C@@H]1c1nc3ccc(Cl)cc3n12 ; C#CC1(C)CCN(C(=O)OC(C)(C)C)C1 ; [2H]C([2H])([2H])N1C(=O)c2cccc(OC(F)F)c2[C@H]2C[C@@H]1c1nc3ccc(C#CC4(C)CCN(C(=O)OC(C)(C)C)C4)cc3n12]

To a stirred mixture of (7R,14R)-11-chloro-1-(difluoromethoxy)-6-(methyl-d₃)-6,7-dihydro-7,14-methanobenzo[f]benzo[4,5]imidazo[1,2-a][1,4]diazocin-5(14H)-one (400 mg, 1.018 mmol) and tert-butyl 3-ethynyl-3-methylpyrrolidine-1-carboxylate (426 mg, 2.036 mmol) in ACN (8 mL) were added K₂CO₃ (703 mg, 5.090 mmol), SPhos (84 mg, 0.204 mmol) and SPhos Pd Gen.3 (159 mg, 0.204 mmol) at room temperature. The resulting mixture was stirred for 1 h at 80° C. under nitrogen atmosphere. The resulting mixture was diluted with water (20 mL) and extracted with EtOAc (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 5% to 95% gradient in 40 min; detector, 254 nm to afford tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)-3-methylpyrrolidine-1-carboxylate (480 mg, 83%) as a yellow solid. MS ESI calculated for C₃₁H₂₉D₃F₂N₄O₄[M+H]⁺, 566.26 found 566.20.

### Preparation 189B: (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5(14H)-one

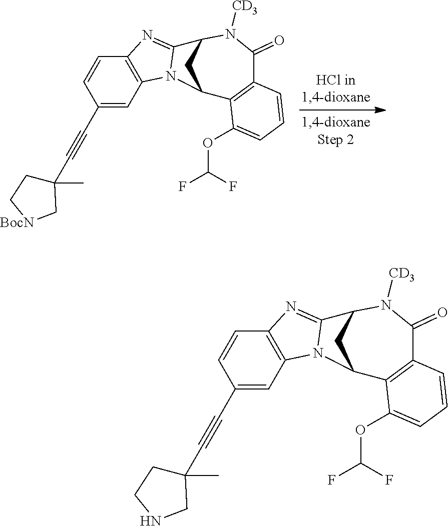 [SMILES: [2H]C([2H])([2H])N1C(=O)c2cccc(OC(F)F)c2[C@H]2C[C@@H]1c1nc3ccc(C#CC4(C)CCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4(C)CCNC4)cc3n12]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylpyrrolidine-1-carboxylate (380 mg, 0.672 mmol) in 1,4-dioxane (5 mL) was added HCl in 1,4-dioxane (5 mL, 4.00 mol/L) dropwise at room temperature. The resulting mixture was stirred for 1 h at room temperature. The resulting mixture was concentrated under reduced pressure. The resulting mixture was diluted with sat. NaHCO₃(aq.) (20 mL) extracted with EtOAc (3×10 mL). The combined organic layers were washed with sat. NaCl (aq.) (3×10 mL), dried over anhydrous Na₂SO₄. After filtration, the filtrate was concentrated under reduced pressure to afford (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5(14H)-one (170 mg, crude) as a yellow solid. MS ESI calculated for C₂₆H₂₁D₃F₂N₄O₂ [M+H]⁺, 466.21 found 466.25.

### Example 189: (7R,14R)-11-((1-(cyclopropanecarbonyl)-3-methylpyrrolidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

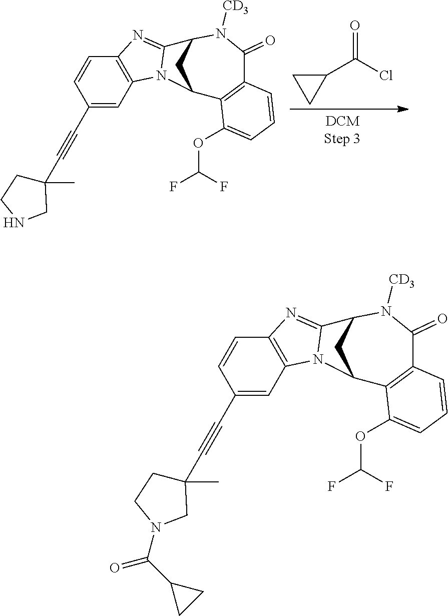

To a stirred solution of (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5(14H)-one (50 mg, 0.107 mmol) in DCM (0.5 mL) was added cyclopropanecarbonyl chloride (12 mg, 0.118 mmol) at room temperature under nitrogen atmosphere. The resulting mixture was stirred for 1 h at room temperature. The resulting mixture was concentrated under reduced pressure and purified by reverse phase flash with the following conditions (Column: XBridge Prep OBD C18 Column 30*150 mm, 5m; Mobile Phase A: Water (10 mmol/L NH₄HCO₃), Mobile Phase B: ACN; Flow rate: 60 mL/min mL/min; Gradient: 29% B to 47% B in 10 min; Wave Length: 254 nm; RT1(min): 10.25) to afford (7R,14R)-11-((1-(cyclopropanecarbonyl)-3-methylpyrrolidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (7 mg, 11%) as a white solid. MS ESI calculated for C₃₀H₂₅D₃F₂N₄O₃[M+H]⁺, 534.23 found 534.15. ¹H NMR (400 MHz, Chloroform-d) δ 8.50-8.45 (m, 1H), 7.66-7.59 (m, 1H), 7.51-7.47 (m, 1H), 7.42 (t, J=8.2 Hz, 1H), 7.37-7.32 (m, 1H), 7.28-7.26 (m, 1H), 7.08-6.68 (m, 1H), 6.21 (d, J=6.5 Hz, 1H), 5.01-4.91 (m, 1H), 3.95-3.38 (m, 5H), 2.86 (d, J=13.4 Hz, 1H), 2.36-2.19 (m, 1H), 2.05-1.97 (m, 1H), 1.69-1.54 (m, 1H), 1.48 (d, J=10.2 Hz, 3H), 1.07-0.95 (m, 2H), 0.82-0.72 (m, 2H).

### Example 190: (7R,14R)-1-(difluoromethoxy)-6-(methyl-d₃)-11-((3-methyl-1-pivaloylpyrrolidin-3-yl)ethynyl)-6,7-dihydro-7,14-methanobenzo[f]benzo[4,5]imidazo[1,2-a][1,4]diazocin-5(14H)-one

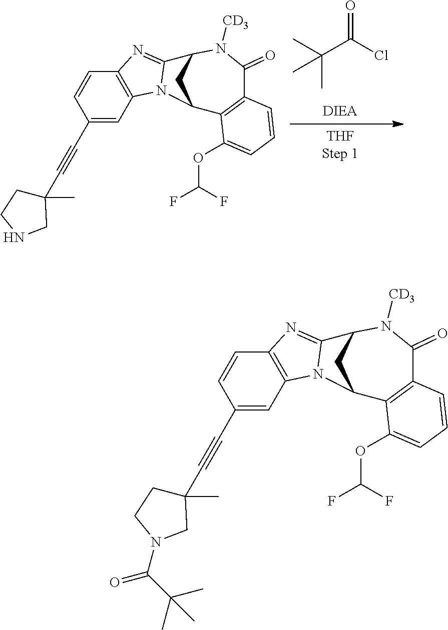

To a stirred solution of (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5(14H)-one (50 mg, 0.107 mmol) and DIEA (37 uL, 0.214 mmol) in THF (2 mL) was added 2,2-dimethylpropanoyl chloride (19 mg, 0.161 mmol) dropwise at room temperature. The resulting mixture was stirred for 1 h at room temperature under nitrogen atmosphere. The resulting mixture was concentrated under reduced pressure and purified by reverse phase flash with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 34% B to 54% B in 10 min; Wave Length: 254/220 nm; RT1(min): 8.57) to afford (7R,14R)-1-(difluoromethoxy)-6-(methyl-d₃)-11-((3-methyl-1-pivaloylpyrrolidin-3-yl)ethynyl)-6,7-dihydro-7,14-methanobenzo[f]benzo[4,5]imidazo[1,2-a][1,4]diazocin-5(14H)-one (15 mg, 25%) as a white solid. MS ESI calculated for C₃₁H₂₉D₃F₂N₄O₃[M+H]⁺, 550.26 found 550.35. ¹H NMR (400 MHz, Chloroform-d) δ 8.51-8.42 (m, 1H), 7.64-7.56 (m, 1H), 7.48-7.38 (m, 2H), 7.37-7.31 (m, 1H), 7.25-7.21 (m, 1H), 7.10-6.66 (m, 1H), 6.25-6.15 (m, 1H), 5.08-4.87 (m, 1H), 3.94-3.60 (m, 3H), 3.52-3.39 (m, 2H), 2.86 (d, J=12.9 Hz, 1H), 2.03-1.65 (m, 2H), 1.45 (s, 3H), 1.30-1.18 (m, 9H). ¹⁹F NMR (377 MHz, Chloroform-d) δ −80.15, −80.60, −81.26, −81.66.

### Example 191: (7R,14R)-1-(difluoromethoxy)-11-(4-hydroxy-3,3-dimethylbut-1-yn-1-yl)-6-(methyl-d3)-6,7-dihydro-7,14-methanobenzo[f]benzo[4,5]imidazo[1,2-a][1,4]diazocin-5(14H)-one

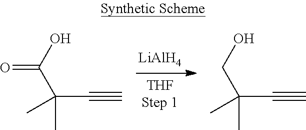

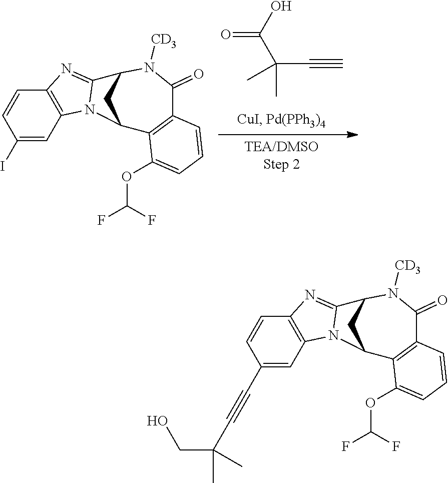

### Preparation 191A: 2,2-dimethylbut-3-yn-1-ol

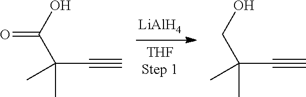

To a stirred solution of 2,2-dimethylbut-3-ynoic acid (600 mg, 5.351 mmol) in THF (6 mL) was added LiAlH₄ (4 mL, 8.027 mmol, 1 M in THF) dropwise at 0° C. under nitrogen atmosphere. The resulting mixture was stirred for 1 h at room temperature. The reaction was quenched with water at 0° C. The resulting mixture was extracted with CH₂Cl₂ (3×20 mL). The combined organic layers were washed with brine (1×20 mL), dried over anhydrous Na₂SO₄. After filtration, the filtrate was concentrated under reduced pressure to afford 2,2-dimethylbut-3-yn-1-ol (500 mg, crude) as a light yellow liquid. MS ESI calculated for C₆H₁₀O [M+H]⁺, 99.07, found N/A. ¹H NMR (400 MHz, Chloroform-d) δ 3.42 (s, 2H), 2.17 (s, 1H), 1.23 (s, 6H).

### Example 191: (7R,14R)-1-(difluoromethoxy)-11-(4-hydroxy-3,3-dimethylbut-1-yn-1-yl)-6-(methyl-d3)-6,7-dihydro-7,14-methanobenzo[f]benzo[4,5]imidazo[1,2-a][1,4]diazocin-5(14H)-one

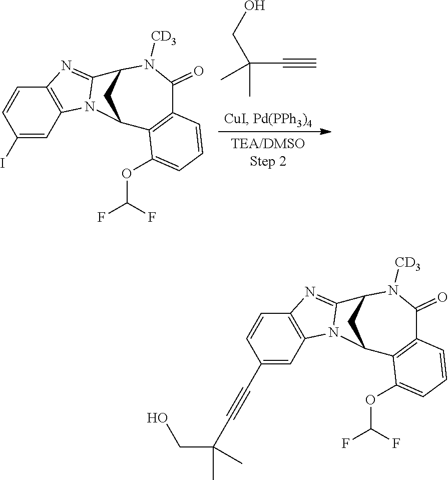

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (70 mg, 0.145 mmol) and 2,2-dimethylbut-3-yn-1-ol (71 mg, 0.725 mmol) in TEA (0.5 mL) and DMSO (0.5 mL) were added CuI (6 mg, 0.029 mmol) and Pd(PPh₃)₄(17 mg, 0.014 mmol) at room temperature. The resulting mixture was stirred for 2 h at 80° C. under nitrogen atmosphere. The mixture was allowed to cool down to room temperature. The resulting mixture was filtered, the filter cake was washed with CH₂Cl₂/MeOH (3×5 mL, 10/1). The filtrate was concentrated under reduced pressure. The residue was purified by Prep-TLC (CH₂Cl₂/MeOH 10/1) followed by reversed-phase flash chromatography with the following conditions: column, C18 silica gel; mobile phase, CH₃CN in Water (10 mmol/L NH₄HCO₃), 30% to 50% gradient in 20 min; detector, 254/220 nm to afford (7R,14R)-1-(difluoromethoxy)-11-(4-hydroxy-3,3-dimethylbut-1-yn-1-yl)-6-(methyl-d3)-6,7-dihydro-7,14-methanobenzo[f]benzo[4,5]imidazo[1,2-a][1,4]diazocin-5(14H)-one (39 mg, 59%) as a white solid. MS ESI calculated for C₂₅H₂₀D₃F₂N₃O₃[M+H]⁺, 455.19. found 455.25. ¹H NMR (400 MHz, Chloroform-d) δ 8.53-8.44 (m, 1H), 7.61 (d, J=8.5 Hz, 1H), 7.51 (d, J=1.6 Hz, 1H), 7.43 (t, J=8.2 Hz, 1H), 7.34 (d, J=8.2 Hz, 1H), 7.30-7.27 (m, 1H), 6.86 (t, J=72.2 Hz, 1H), 6.22 (d, J=7.3 Hz, 1H), 4.99 (d, J=7.2 Hz, 1H), 3.51 (s, 2H), 3.49-3.38 (m, 1H), 2.86 (d, J=13.5 Hz, 1H), 1.32 (s, 6H). ¹⁹F NMR (377 MHz, Chloroform-d) δ −80.10, −80.55, −81.39, −81.84.

### Example 192: (7R,14R)-1-(difluoromethoxy)-11-((1-(2-hydroxy-2-methylpropanoyl)-3-methylpyrrolidin-3-yl)ethynyl)-6-(methyl-d₃)-6,7-dihydro-7,14-methanobenzo[f]benzo[4,5]imidazo[1,2-a][1,4]diazocin-5(14H)-one

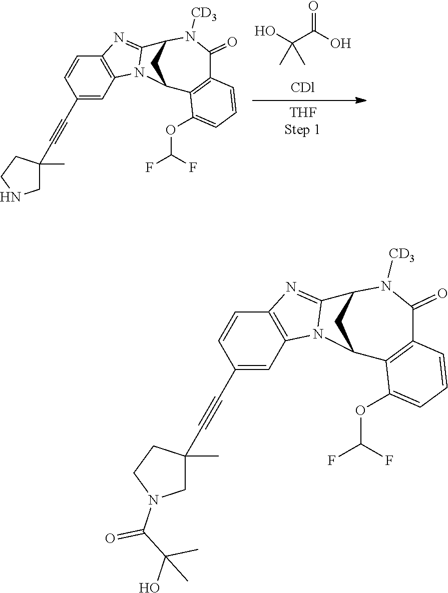

To a stirred solution of 2-hydroxy-2-methylpropanoic acid (17 mg, 0.161 mmol) in THF (2 mL) was added CDI (35 mg, 0.214 mmol) at room temperature. The resulting mixture was stirred for 15 min at room temperature under nitrogen atmosphere. To the above mixture was added (7R,14R)-1-(difluoromethoxy)-6-(methyl-d₃)-11-((3-methylpyrrolidin-3-yl)ethynyl)-6,7-dihydro-7,14-methanobenzo[f]benzo[4,5]imidazo[1,2-a][1,4]diazocin-5(14H)-one (50 mg, 0.107 mmol) at room temperature. The resulting mixture was stirred for additional 1 h at room temperature. The resulting mixture was concentrated under reduced pressure and purified by reverse phase flash with the following conditions (Column: Sunfire prep C18 column, 30*150 mm, 5m; Mobile Phase A: Water (0.1% FA), Mobile Phase B: ACN; Flow rate: 60 mL/min mL/min; Gradient: 25% B to 42% B in 10 min; Wave Length: 254 nm/220 nm; RT1(min): 12.2) to afford (7R,14R)-1-(difluoromethoxy)-11-((1-(2-hydroxy-2-methylpropanoyl)-3-methylpyrrolidin-3-yl)ethynyl)-6-(methyl-d₃)-6,7-dihydro-7,14-methanobenzo[f]benzo[4,5]imidazo[1,2-a][1,4]diazocin-5(14H)-one (8 mg, 13%) as a white solid. MS ESI calculated for C₃₀H₂₇D₃F₂N₄O₄ [M+H]⁺, 552.24. found 552.25. ¹H NMR (400 MHz, Chloroform-d) δ 8.59-8.40 (m, 1H), 7.62 (d, J=8.4 Hz, 1H), 7.51-7.30 (m, 3H), 7.26-7.18 (m, 1H), 7.12-6.57 (m, 1H), 6.21 (d, J=7.1 Hz, 1H), 4.98 (d, J=6.9 Hz, 1H), 4.05-3.64 (m, 3H), 3.51-3.39 (m, 2H), 2.86 (d, J=13.5 Hz, 1H), 2.36-2.19 (m, 1H), 2.08-1.81 (m, 1H), 1.57-1.36 (m, 9H).

### Example 193: 3-(3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)azetidin-1-yl)-2,2-dimethyl-3-oxopropanenitrile

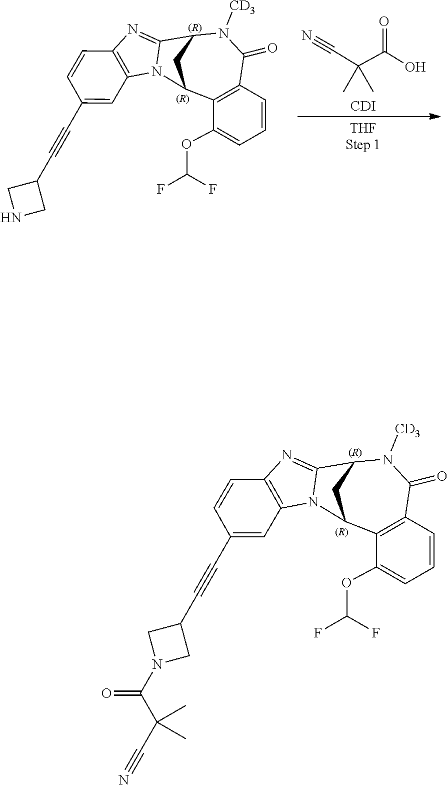

A mixture of 2-cyano-2,2-dimethylacetic acid (21 mg, 0.182 mmol) and CDI (30 mg, 0.182 mmol) in THF (0.5 mL) was stirred for 10 min at room temperature. To the above mixture was added (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol). The resulting mixture was stirred for additional 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in 3-(3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)azetidin-1-yl)-2,2-dimethyl-3-oxopropanenitrile (21 mg, 43%) as a white solid. MS ESI calculated for C₂₉H₂₂D₃F₂N₅O₃ [M+H]⁺, 533.21. found 533.15. ¹H NMR (400 MHz, Chloroform-d) δ 8.58-8.45 (m, 1H), 7.69 (d, J=8.5 Hz, 1H), 7.56 (s, 1H), 7.47 (d, J=8.2 Hz, 1H), 7.41-7.32 (m, 2H), 6.86 (t, J=72.6 Hz, 1H), 6.27 (d, J=6.8 Hz, 1H), 5.10 (d, J=6.5 Hz, 1H), 4.87 (t, J=8.8 Hz, 1H), 4.63 (t, J=7.7 Hz, 1H), 4.40 (t, J=9.4 Hz, 1H), 4.24-4.14 (m, 1H), 3.80-3.66 (m, 1H), 3.56-3.36 (m, 1H), 2.90 (d, J=13.4 Hz, 1H), 1.59 (s, 6H). ¹⁹F NMR (377 MHz, Chloroform-d) δ −80.17, −80.61, −81.06, −81.50.

### Example 194: (7R,14R)-11-((1-(2,2-difluorocyclopropane-1-carbonyl)azetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

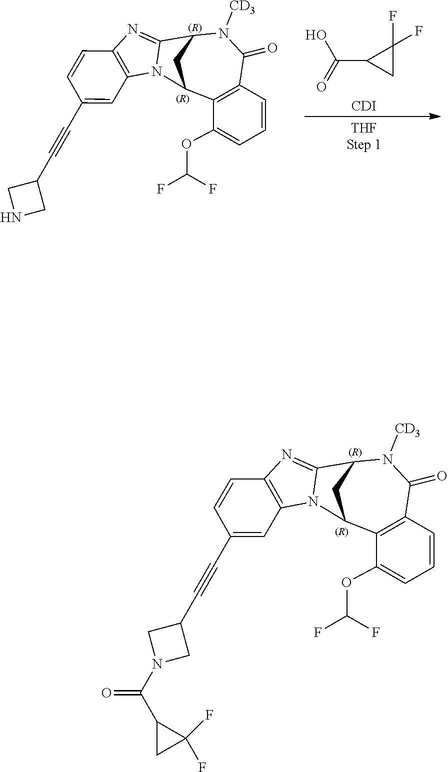

A mixture of 2,2-difluorocyclopropane-1-carboxylic acid (22 mg, 0.182 mmol) and CDI (30 mg, 0.182 mmol) in THF (0.5 mL) was stirred for 10 min at room temperature. To the above mixture was added (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in (7R,14R)-11-((1-(2,2-difluorocyclopropane-1-carbonyl)azetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (36 mg, 73%) as a white solid. MS ESI calculated for C₂₈H₁₉D₃F₄N₄O₃ [M+H]⁺, 542.18. found 542.15. ¹H NMR (400 MHz, Chloroform-d) δ 8.49 (d, J=8.1 Hz, 1H), 7.67 (d, J=8.4 Hz, 1H), 7.60-7.52 (m, 1H), 7.46 (d, J=8.2 Hz, 1H), 7.39-7.31 (m, 2H), 7.09-6.63 (m, 1H), 6.29-6.21 (m, 1H), 5.05 (d, J=6.8 Hz, 1H), 4.63-4.52 (m, 1H), 4.45-4.32 (m, 2H), 4.26-4.11 (m, 1H), 3.78-3.65 (m, 1H), 3.55-3.44 (m, 1H), 2.89 (d, J=13.5 Hz, 1H), 2.31-2.12 (m, 2H), 1.76-1.62 (m, 1H).

### Example 195: (7R,14R)-1-(difluoromethoxy)-11-((1-(1-hydroxycyclopropane-1-carbonyl)azetidin-3-yl)ethynyl)-6-(methyl-d3)-6,7-dihydro-7,14-methanobenzo[f]benzo[4,5]imidazo[1,2-a][1,4]diazocin-5(14H)-one

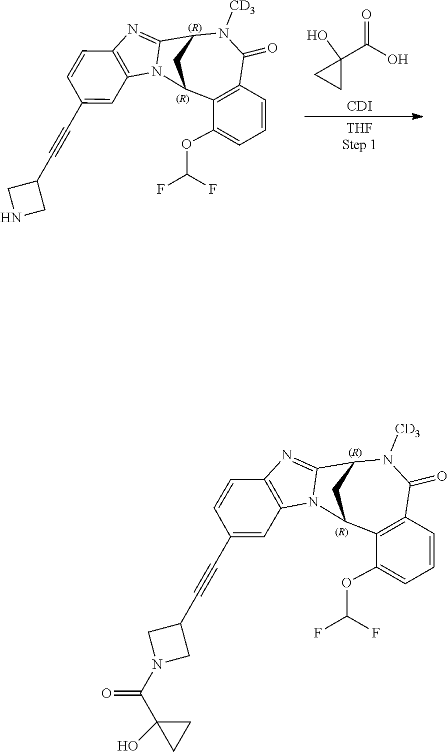

A mixture of 1-hydroxycyclopropane-1-carboxylic acid (19 mg, 0.182 mmol) and CDI (30 mg, 0.182 mmol) in THF (0.5 mL) was stirred for 10 min at room temperature. To the above mixture was added (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in (7R,14R)-1-(difluoromethoxy)-11-((1-(1-hydroxycyclopropane-1-carbonyl)azetidin-3-yl)ethynyl)-6-(methyl-d3)-6,7-dihydro-7,14-methanobenzo[f]benzo[4,5]imidazo[1,2-a][1,4]diazocin-5(14H)-one (28 mg, 59%) as a white solid. MS ESI calculated for C₂₈H₂₁D₃F₂N₄O₄ [M+H]⁺, 522.20. found 522.20. ¹H NMR (400 MHz, Chloroform-d) δ 8.53-8.44 (m, 1H), 7.63 (d, J=8.5 Hz, 1H), 7.54 (s, 1H), 7.46 (d, J=8.2 Hz, 1H), 7.36 (d, J=8.2 Hz, 1H), 7.33-7.28 (m, 1H), 6.87 (t, J=72.6 Hz, 1H), 6.28 (d, J=6.8 Hz, 1H), 5.15 (d, J=6.7 Hz, 1H), 4.89-4.72 (m, 1H), 4.69-4.48 (m, 1H), 4.49-4.27 (m, 1H), 4.24-3.99 (m, 1H), 3.69-3.48 (m, 2H), 2.90 (d, J=13.5 Hz, 1H), 1.40-1.22 (m, 2H), 1.08-0.95 (m, 2H). ¹⁹F NMR (377 MHz, Chloroform-d) δ −80.15, −80.60, −81.09, −81.53.

### Example 196: (7R,14R)-11-((2,2-difluorocyclopropyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

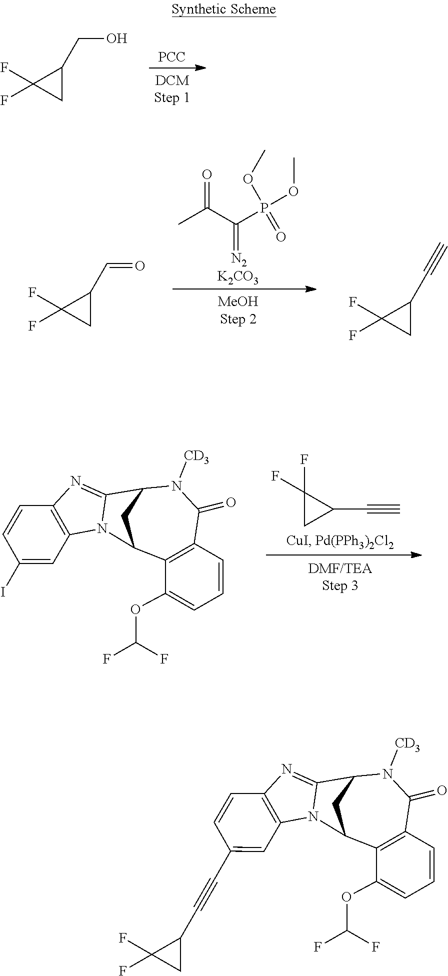

### Preparation 196A: 2,2-difluorocyclopropane-1-carbaldehyde

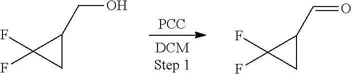

To a stirred mixture of (2,2-difluorocyclopropyl)methanol (1.00 g, 9.252 mmol) in DCM (20 mL) was added PCC (2.99 g, 13.878 mmol) at 0° C. under nitrogen atmosphere. The resulting mixture was stirred for 2 h at room temperature under nitrogen atmosphere. The resulting mixture was diluted with diethyl ether (20 mL). The resulting mixture was filtered, the filter cake was washed with diethyl ether (3×5 mL). The filtrate was concentrated under reduced pressure. This resulted in 2,2-difluorocyclopropane-1-carbaldehyde (900 mg, 92%) as a yellow oil. MS ESI calculated for C₄H₄F₂O [M+H]⁺, 107.02. found N/A. ¹H NMR (400 MHz, Chloroform-d) δ 9.39-9.25 (m, 1H), 2.76-2.62 (m, 1H), 2.24-2.14 (m, 1H), 1.97-1.86 (m, 1H).

### Preparation 196B: 2-ethynyl-1,1-difluorocyclopropane

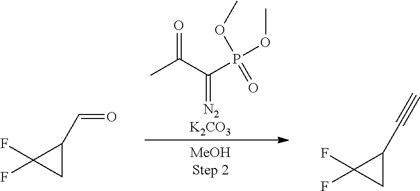

To a stirred mixture of 2,2-difluorocyclopropane-1-carbaldehyde (500 mg, 4.714 mmol) and K₂CO₃ (1.30 g, 9.428 mmol) in MeOH (8 mL) was added seyferth-gilbert homologation (1.60 g, 7.071 mmol) at 0° C. under nitrogen atmosphere. The resulting mixture was stirred for 2 h at room temperature under nitrogen atmosphere. The resulting mixture was diluted with diethyl ether (20 mL). The resulting mixture was washed with 2×10 mL of water. The resulting mixture was concentrated under vacuum. This resulted in 2-ethynyl-1,1-difluorocyclopropane (290 mg, 60%) as a yellow oil. MS ESI calculated for C5H₄F₂[M+H]⁺, 103.03. found N/A.

### Example 196: (7R,14R)-11-((2,2-difluorocyclopropyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

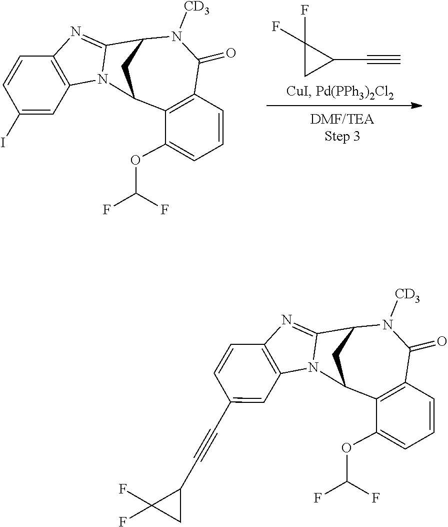

To a stirred mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (60 mg, 0.124 mmol) and 2-ethynyl-1,1-difluorocyclopropane (63 mg, 0.620 mmol) in DMF (1.2 mL) and TEA (1.2 mL) were added CuI (2 mg, 0.012 mmol) and Pd(PPh₃)₂Cl₂ (9 mg, 0.012 mmol) at room temperature. The resulting mixture was stirred for 1 h at 80° C. under nitrogen atmosphere. The resulting mixture was concentrated under vacuum. The residue was purified by silica gel column chromatography, eluted with CH₂Cl₂/MeOH (20/1) followed by reversed-phase flash chromatography with the following conditions: column, C18 silica gel; mobile phase, CH₃CN in Water (10 mmol/L NH₄HCO₃), 30% to 60% gradient in 25 min. The resulting mixture was concentrated under vacuum. This resulted in (7R,14R)-11-((2,2-difluorocyclopropyl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (30 mg, 52%) as a white solid. MS ESI calculated for C₂₄H₁₄D₃F₄N₃O₂ [M+H]⁺, 459.14. found 459.20. ¹H NMR (400 MHz, Chloroform-d) δ 8.48 (d, J=8.1 Hz, 1H), 7.63 (d, J=8.4 Hz, 1H), 7.56-7.50 (m, 1H), 7.47-7.40 (m, 1H), 7.37-7.29 (m, 2H), 7.04-6.64 (m, 1H), 6.22 (d, J=7.0 Hz, 1H), 5.03-4.92 (m, 1H), 3.44 (d, J=8.5 Hz, 1H), 2.86 (d, J=13.6 Hz, 1H), 2.43-2.29 (m, 1H), 1.79-1.74 (m, 1H), 1.70-1.53 (m, 1H). ¹⁹F NMR (377 MHz, Chloroform-d) δ −79.99, −80.45, −81.28, −81.72, −129.26, −129.66, −138.32, −138.72.

### Example 197: (7R,14R)-1-(difluoromethoxy)-11-((1-(2-hydroxy-2-methylpropyl)azetidin-3-yl)ethynyl)-6-(methyl-d3)-6,7-dihydro-7,14-methanobenzo[f]benzo[4,5]imidazo[1,2-a][1,4]diazocin-5(14H)-one

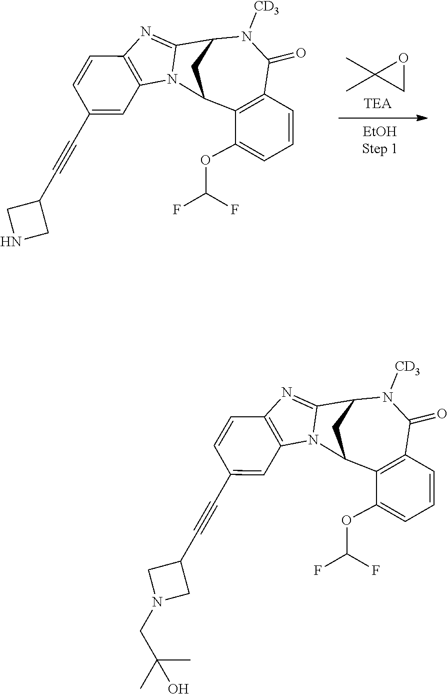

To a stirred solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol) and TEA (38.13 uL, 0.273 mmol) in EtOH (0.5 mL) was added 2,2-dimethyloxirane (10 mg, 0.137 mmol) at room temperature. The resulting mixture was stirred for 3 h at 60° C. The residue was purified by reversed-phase flash chromatography with the following conditions: column, C18 silica gel; mobile phase, CH₃CN in Water (10 mmol/L NH₄HCO₃), 10% to 43% gradient in 10 min to afford (7R,14R)-1-(difluoromethoxy)-11-((1-(2-hydroxy-2-methylpropyl)azetidin-3-yl)ethynyl)-6-(methyl-d3)-6,7-dihydro-7,14-methanobenzo[f]benzo[4,5]imidazo[1,2-a][1,4]diazocin-5(14H)-one (24 mg, 49%) as a white solid. MS ESI calculated for C₂₈H₂₅D₃F₂N₄O₃[M+H]⁺, 510.23. found 510.25. ¹H NMR (400 MHz, Chloroform-d) δ 8.52-8.44 (m, 1H), 7.65-7.58 (m, 1H), 7.54-7.47 (m, 1H), 7.47-7.39 (m, 1H), 7.37-7.32 (m, 1H), 7.29-7.27 (m, 1H), 7.26-7.25 (m, 1H), 6.84 (t, J=72.7 Hz, 1H), 6.23-6.13 (m, 1H), 4.93 (d, J=7.1 Hz, 1H), 4.05-3.93 (m, 2H), 3.67-3.56 (m, 1H), 3.55-3.37 (m, 3H), 2.92-2.80 (m, 1H), 2.63 (s, 2H), 1.19 (s, 6H). ¹⁹F NMR (377 MHz, Chloroform-d) δ −79.94, −80.39, −80.99, −81.43.

### Example 198: (7R,14R)-11-((1-(2,2-difluoroethyl)azetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

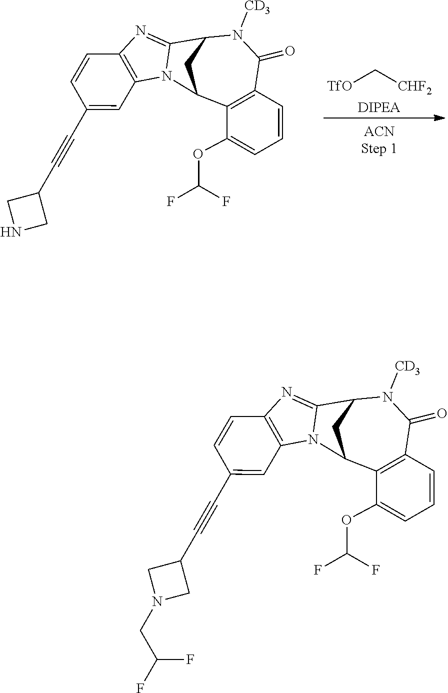

To a stirred solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol) and DIPEA (32 uL, 0.182 mmol) in ACN (0.5 mL) was added 2,2-difluoroethyl trifluoromethanesulfonate (25 mg, 0.118 mmol) at room temperature. The resulting mixture was stirred for 3 h at room temperature. The residue was purified by reversed-phase flash chromatography with the following conditions: column, C18 silica gel; mobile phase, CH₃CN in Water (10 mmol/L NH₄HCO₃), 10% to 45% gradient in 10 min to afford (7R,14R)-11-((1-(2,2-difluoroethyl)azetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (25 mg, 53%) as a white solid. MS ESI calculated for C₂₆H₁₉D₃F₄N₄O₂ [M+H]⁺, 502.19. found 502.10. ¹H NMR (400 MHz, Chloroform-d) δ 8.52-8.45 (m, 1H), 7.63 (d, J=8.4 Hz, 1H), 7.54-7.48 (m, 1H), 7.43 (t, J=8.2 Hz, 1H), 7.37-7.32 (m, 1H), 7.30-7.28 (m, 1H), 6.85 (t, J=72.7 Hz, 1H), 6.21 (d, J=7.2 Hz, 1H), 6.10-5.82 (m, 1H), 4.94 (d, J=7.1 Hz, 1H), 4.08 (s, 2H), 3.78-3.50 (m, 3H), 3.50-3.39 (m, 1H), 3.17-2.97 (m, 2H), 2.86 (d, J=13.6 Hz, 1H). ¹⁹F NMR (377 MHz, Chloroform-d) δ −80.00, −80.44, −80.89, −81.35, −120.80.

### Example 199 and 200: (7R,14R)-1-(difluoromethoxy)-6-(methyl-d3)-11-((R or S)-4,4,4-trifluoro-3-hydroxybut-1-yn-1-yl)-6,7-dihydro-7,14-methanobenzo[f]benzo[4,5]imidazo[1,2-a][1,4]diazocin-5(14H)-one and (7R,14R)-1-(difluoromethoxy)-6-(methyl-d3)-11-((S or R)-4,4,4-trifluoro-3-hydroxybut-1-yn-1-yl)-6,7-dihydro-7,14-methanobenzo[f]benzo[4,5]imidazo[1,2-a][1,4]diazocin-5(14H)-one

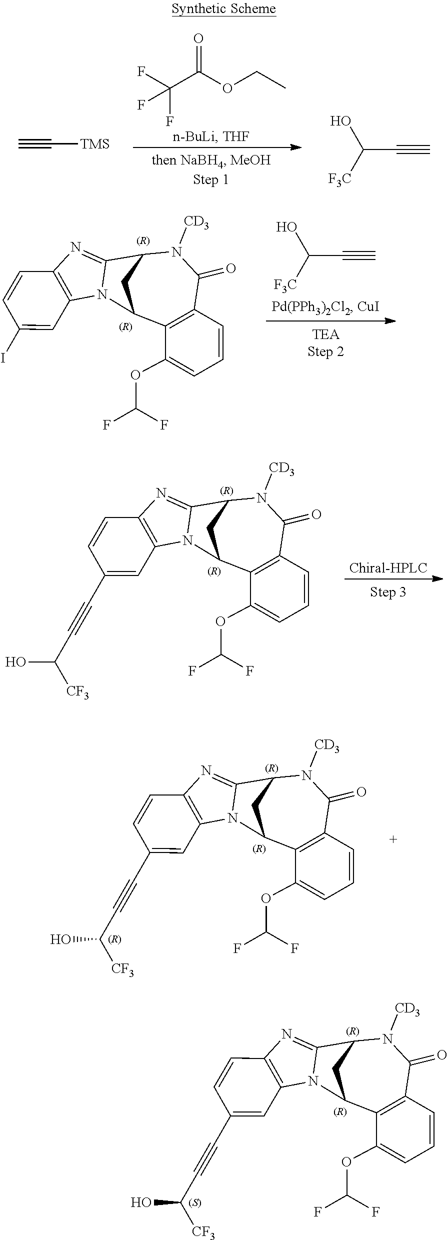

### Preparation 199A: 1,1,1-trifluorobut-3-yn-2-ol

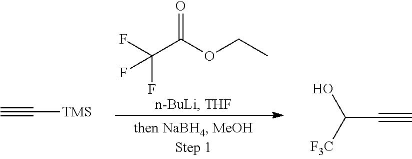

A solution of trimethylsilylacetylene (4.2 mL, 29.719 mmol) in THF (40 mL) was treated with n-BuLi (13.7 mL, 34.177 mmol) for 1 h at −78° C. under nitrogen atmosphere followed by the addition of trifluoroethyl acetate (5.3 mL, 44.579 mmol) dropwise at −78° C. The resulting mixture was stirred for 1 h at room temperature under nitrogen atmosphere. The resulting mixture was diluted with MeOH (40 mL) and cooled to 0° C., NaBH₄ (2.25 g, 60.000 mmol) was added to it in small portions. The resulting mixture was stirred overnight at room temperature under nitrogen atmosphere. The reaction was quenched by the addition of sat. NH₄Cl (aq.) (60 mL) at 0° C. The resulting mixture was extracted with ether (3×40 mL). The combined organic layers were washed with brine (20 mL), dried over anhydrous Na₂SO₄. After filtration, the filtrate was concentrated under reduced pressure to afford 1,1,1-trifluorobut-3-yn-2-ol (4.14 g, crude) as a yellow oil. MS ESI calculated for C₄H₃F₃O [M+H]⁺, 125.01 found N/A. ¹⁹F NMR (377 MHz, Chloroform-d) δ −79.91.

### Preparation 199B: (7R,14R)-1-(difluoromethoxy)-6-(methyl-d3)-11-(4,4,4-trifluoro-3-hydroxybut-1-yn-1-yl)-6,7-dihydro-7,14-methanobenzo[f]benzo[4,5]imidazo[1,2-a][1,4]diazocin-5(14H)-one

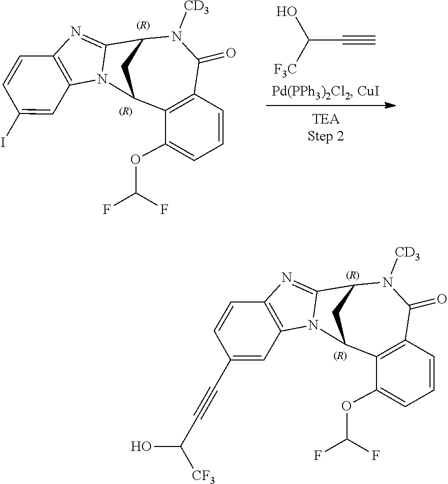

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (200 mg, 0.457 mmol) and 1,1,1-trifluorobut-3-yn-2-ol (660 mg, 5.316 mmol) in TEA (4 mL) were added CuI (17 mg, 0.09 mmol) and Pd(PPh₃)₂Cl₂ (32 mg, 0.046 mmol) at room temperature. The resulting mixture was stirred for 3 h at 80° C. under nitrogen atmosphere. The mixture was concentrated under reduce pressure and purified by silica gel column chromatography, eluted with CH₂Cl₂/MeOH (10/1) followed by reversed-phase flash chromatography with the following conditions: column, C18 silica gel; mobile phase, CH₃CN in water (10 mmol/L NH₄HCO₃), 5% to 47% gradient in 20 min to afford (7R,14R)-1-(difluoromethoxy)-6-(methyl-d3)-11-(4,4,4-trifluoro-3-hydroxybut-1-yn-1-yl)-6,7-dihydro-7,14-methanobenzo[f]benzo[4,5]imidazo[1,2-a][1,4]diazocin-5(14H)-one (49 mg, 25%) as a white solid. MS ESI calculated for C₂₃H₁₃D₃F₅N₃O₃[M+H]⁺, 481.13. found 481.15

### Example 199 and 200: (7R,14R)-1-(difluoromethoxy)-6-(methyl-d3)-11-((R or S)-4,4,4-trifluoro-3-hydroxybut-1-yn-1-yl)-6,7-dihydro-7,14-methanobenzo[f]benzo[4,5]imidazo[1,2-a][1,4]diazocin-5(14H)-one and (7R,14R)-1-(difluoromethoxy)-6-(methyl-d3)-11-((S or R)-4,4,4-trifluoro-3-hydroxybut-1-yn-1-yl)-6,7-dihydro-7,14-methanobenzo[f]benzo[4,5]imidazo[1,2-a][1,4]diazocin-5(14H)-one

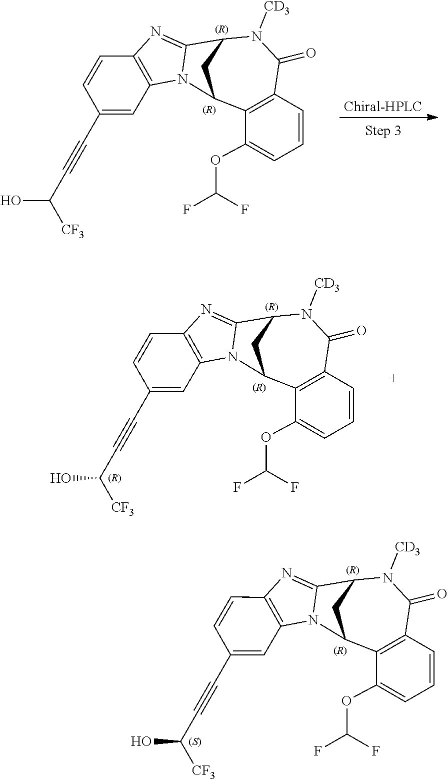

The product was purified by Prep-CHIRAL-HPLC with the following conditions (Column: CHIRALPAK PAK AD-H, 30*250 mm; Mobile Phase A: Hex (10 mM NH₃-MeOH), Mobile Phase B: EtOH; Flow rate: 40 mL/min; Gradient: isocratic 10; Wave Length: 224/272 nm; RT1(min): 16.08; RT2(min): 25.57). The first peak afforded (11 mg, 23%) as a white solid. MS ESI calculated for C₂₃H₁₃D₃F₅N₃O₃[M+H]⁺, 481.13. found 481.05. ¹H NMR (400 MHz, DMSO-d₆) δ 8.31-8.24 (m, 1H), 7.78-7.38 (m, 5H), 7.31-7.25 (m, 1H), 7.11 (d, J=7.1 Hz, 1H), 6.25 (d, J=7.1 Hz, 1H), 5.30-5.21 (m, 2H), 3.57-3.44 (m, 1H), 2.82 (d, J=13.8 Hz, 1H). ¹⁹F NMR (377 MHz, DMSO-d) δ −77.73, −82.18, −82.21. The second peak afforded (13 mg, 26%) as a white solid. MS ESI calculated for C₂₃H₁₃D₃F₅N₃O₃[M+H]⁺, 481.13. found 481.05. ¹H NMR (400 MHz, DMSO-d₆) δ 8.28 (d, J=7.3 Hz, 1H), 7.77-7.37 (m, 5H), 7.28 (d, J=8.4 Hz, 1H), 7.10 (d, J=7.1 Hz, 1H), 6.25 (d, J=7.1 Hz, 1H), 5.31-5.15 (m, 2H), 3.52-3.44 (m, 1H), 2.81 (d, J=13.8 Hz, 1H). ¹⁹F NMR (377 MHz, DMSO-d₆) δ −77.73, −82.17, −82.19.

### Example 201: (7R,14R)-11-((1-(cyclopropanecarbonyl)pyrrolidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

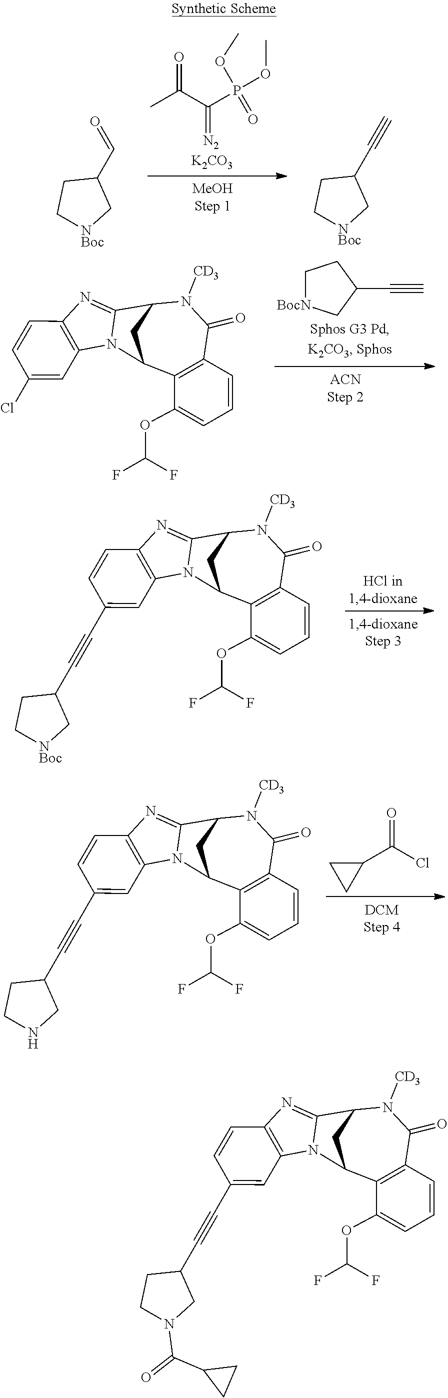

### Preparation 201A: tert-butyl 3-ethynylpyrrolidine-1-carboxylate

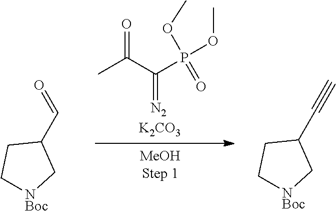

To a stirred solution of tert-butyl 3-formylpyrrolidine-1-carboxylate (3.00 g, 15.06 mmol) in MeOH (20 mL) were added seyferth-gilbert homologation (3.39 mL, 22.58 mmol) and K₂CO₃ (4.99 g, 36.13 mmol) at 0° C. The resulting mixture was stirred for 3 h at 25° C. The resulting mixture was diluted with EtOAc (50 mL). The resulting mixture was filtered, the filter cake was washed with EtOAc (2×30 mL). The filtrate was concentrated under reduced pressure. The residue was purified by reversed-phase flash chromatography with the following conditions: column, C18 silica gel; mobile phase, CH₃CN in Water (0.1% FA), 10% to 50% gradient in 10 min. This resulted in tert-butyl 3-ethynylpyrrolidine-1-carboxylate (2.10 g, 71%) as a white solid. MS ESI calculated for C₁₁H₁₇NO₂ [M+H]⁺, 196.13 found N/A. ¹H NMR (400 MHz, Chloroform-d) 3.68-3.58 (m, 1H), 3.57-3.46 (m, 1H), 3.39-3.25 (m, 2H), 2.99-2.90 (m, 1H), 2.20-2.09 (m, 2H), 2.00-1.89 (m, 1H), 1.46 (s, 9H).

### Preparation 201B: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate

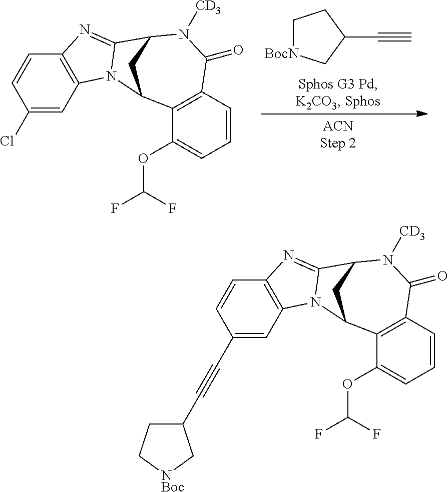 [SMILES: [2H]C([2H])([2H])N1C(=O)c2cccc(OC(F)F)c2[C@H]2C[C@@H]1c1nc3ccc(Cl)cc3n12 ; C#CC1CCN(C(=O)OC(C)(C)C)C1 ; [2H]C([2H])([2H])N1C(=O)c2cccc(OC(F)F)c2[C@H]2C[C@@H]1c1nc3ccc(C#CC4CCN(C(=O)OC(C)(C)C)C4)cc3n12]

A solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (1.00 g, 2.55 mmol), tert-butyl 3-ethynylpyrrolidine-1-carboxylate (0.99 g, 5.09 mmol), SPhos (209 mg, 0.51 mmol), SPhos Pd Gen.3 (397 mg, 0.51 mmol) and K₂CO₃ (1.76 g, 12.73 mmol) in ACN (15 mL) was stirred for 2 h at 80° C. under nitrogen atmosphere. The reaction was quenched by the addition of water (50 mL) at room temperature. The resulting mixture was extracted with EtOAc (2×80 mL). The combined organic layers were washed with brine (2×50 mL), dried over anhydrous Na₂SO₄. After filtration, the filtrate was concentrated under reduced pressure. The residue was purified by silica gel column chromatography, eluted with DCM/MeOH (10/1) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (1.20 g, 85%) as a brown solid. MS ESI calculated for C₃₀H₂₇D₃F₂N₄O₄ [M+H]⁺, 552.24 found 552.30. ¹H NMR (400 MHz, Chloroform-d) δ 8.56-8.43 (m, 1H), 7.69-7.58 (m, 1H), 7.54-7.29 (m, 4H), 7.08-6.58 (m, 1H), 6.35-6.16 (m, 1H), 5.05-4.92 (m, 1H), 3.80-3.51 (m, 3H), 3.49-3.31 (m, 3H), 3.25-3.10 (m, 1H), 2.94-2.78 (m, 1H), 2.30-2.18 (m, 1H), 1.48 (s, 9H).

Preparation 201C: (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one

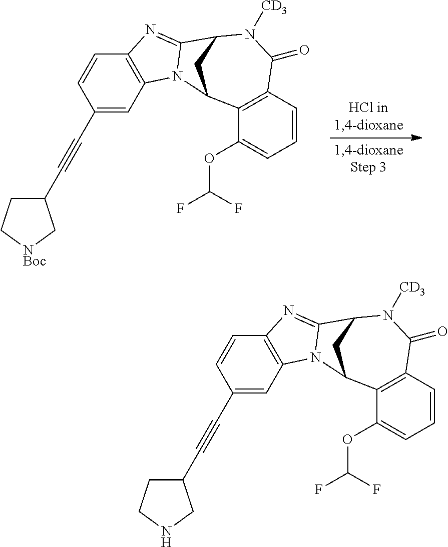 [SMILES: [2H]C([2H])([2H])N1C(=O)c2cccc(OC(F)F)c2[C@H]2C[C@@H]1c1nc3ccc(C#CC4CCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4CCNC4)cc3n12]

A solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrrolidine-1-carboxylate (1.20 g, 2.18 mmol) in 1,4-dioxane (10 mL) was added HCl (4 M in 1,4-dioxane, 10 mL) was stirred for 1 h at room temperature. The mixture was neutralized to pH 7 with saturated NaHCO₃(aq.). The resulting mixture was diluted with water (50 mL). The resulting mixture was extracted with EtOAc (2×80 mL). The combined organic layers were washed with brine (2×50 mL), dried over anhydrous Na₂SO₄. After filtration, the filtrate was concentrated under reduced pressure. This resulted in (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (400 mg, crude) as a brown yellow oil. MS ESI calculated for C₂₅H₁₉D₃F₂N₄O₂ [M+H]⁺, 452.19 found 452.35.

### Example 201: (7R,14R)-11-((1-(cyclopropanecarbonyl)pyrrolidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

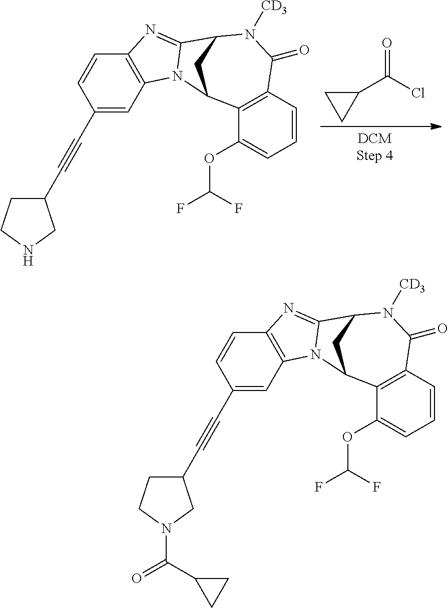

A solution of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (60 mg, 0.13 mmol) and cyclopropanecarbonyl chloride (17 mg, 0.16 mmol) in DCM (1 mL) was stirred for 1 h at room temperature. The reaction was quenched by the addition of water (10 mL) at room temperature. The resulting mixture was extracted with EtOAc (2×20 mL). The combined organic layers were washed with brine (2×10 mL), dried over anhydrous Na₂SO₄. After filtration, the filtrate was concentrated under reduced pressure and purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min. This resulted in (7R,14R)-11-((1-(cyclopropanecarbonyl)pyrrolidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (9 mg, 13%) as a white solid. MS ESI calculated for C29H₂₃D₃F₂N₄O₃[M+H]⁺, 520.22. found 520.30. ¹H NMR (400 MHz, Chloroform-d) 8.49 (d, J=8.1 Hz, 1H), 7.67-7.60 (m, 1H), 7.54-7.49 (m, 1H), 7.43 (t, J=8.2 Hz, 1H), 7.38-7.32 (m, 1H), 7.31-7.27 (m, 1H), 6.86 (t, J=72.7 Hz, 1H), 6.22 (d, J=6.4 Hz, 1H), 5.03-4.94 (m, 1H), 4.05-3.65 (m, 3H), 3.59-3.42 (m, 2H), 3.38-3.18 (m, 1H), 2.87 (d, J=13.2 Hz, 1H), 2.35-2.05 (m, 2H), 1.66-1.59 (m, 1H), 1.09-0.97 (m, 2H), 0.86-0.72 (m, 2H). ¹⁹F NMR (377 MHz, Chloroform-d) δ −79.99, −80.03, −80.44, −80.48, −81.01, −81.10, −81.26, −81.28, −81.45, −81.54, −81.70, −81.72.

### Example 202: (7R,14R)-1-(difluoromethoxy)-11-((1-(2-hydroxy-2-methylpropanoyl)pyrrolidin-3-yl)ethynyl)-6-(methyl-d3)-6,7-dihydro-7,14-methanobenzo[f]benzo[4,5]imidazo[1,2-a][1,4]diazocin-5(14H)-one

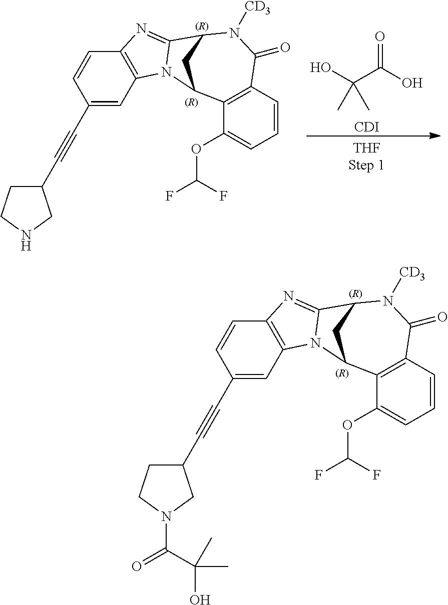

A solution of 2-hydroxy-2-methylpropanoic acid (28 mg, 0.27 mmol,) in THF (1 mL) was treated with CDI (43 mg, 0.27 mmol) for 10 min at room temperature followed by the addition of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (60 mg, 0.13 mmol) in portions at room temperature. The resulting mixture was stirred for additional 1 h at room temperature. The reaction was quenched by the addition of water (10 mL) at room temperature. The resulting mixture was extracted with EtOAc (2×20 mL). The combined organic layers were washed with brine (2×10 mL), dried over anhydrous Na₂SO₄. After filtration, the filtrate was concentrated under reduced pressure and purified by Prep-TLC (CH₂Cl₂/MeOH 20/1) followed by Prep-HPLC with the following conditions (Column: Xselect CSH F-Phenyl OBD column 30*250 mm, 5 μm; Mobile Phase A: Water (0.1% FA), Mobile Phase B: ACN; Flow rate: 60 mL/min; Gradient: 27% B to 45% B in 10 min; Wave Length: 254 nm; RT1(min): 10.53) to afford (7R,14R)-1-(difluoromethoxy)-11-((1-(2-hydroxy-2-methylpropanoyl)pyrrolidin-3-yl)ethynyl)-6-(methyl-d3)-6,7-dihydro-7,14-methanobenzo[f]benzo[4,5]imidazo[1,2-a][1,4]diazocin-5(14H)-one (14 mg, 19%) as a white solid. MS ESI calculated for C₂₉H₂₅D₃F₂N₄O₄[M+H]⁺, 538.23. found 538.30. ¹H NMR (400 MHz, Chloroform-d) δ 8.51-8.45 (m, 1H), 7.67 (d, J=8.5 Hz, 1H), 7.54-7.49 (m, 1H), 7.45 (t, J=8.2 Hz, 1H), 7.38-7.28 (m, 2H), 6.86 (t, J=72.6 Hz, 1H), 6.24 (d, J=6.4 Hz, 1H), 5.20-5.01 (m, 1H), 3.99-3.74 (m, 2H), 3.74-3.55 (m, 2H), 3.55-3.41 (m, 1H), 3.33-3.24 (m, 2H), 2.89 (d, J=13.3 Hz, 1H), 2.42-1.99 (m, 2H), 1.49 (s, 6H). ¹⁹F NMR (377 MHz, Chloroform-d) δ −80.20, −80.64, −81.04, −81.50.

### Example 203: (7R,14R)-1-(difluoromethoxy)-11-((1-(1-hydroxycyclopropane-1-carbonyl)pyrrolidin-3-yl)ethynyl)-6-(methyl-d3)-6,7-dihydro-7,14-methanobenzo[f]benzo[4,5]imidazo[1,2-a][1,4]diazocin-5(14H)-one

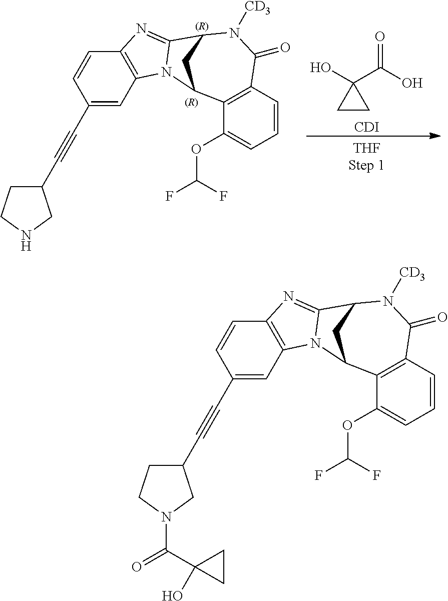

A solution of 1-hydroxycyclopropane-1-carboxylic acid (27 mg, 0.27 mmol) in THF (1 mL) was treated with CDI (43 mg, 0.27 mmol) for 10 min at room temperature followed by the addition of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (50 mg, 0.11 mmol) in portions at room temperature. The resulting mixture was stirred for additional 1 h at room temperature. The reaction was quenched by the addition of water (10 mL) at room temperature. The resulting mixture was extracted with EtOAc (2×20 mL). The combined organic layers were washed with brine (2×10 mL), dried over anhydrous Na₂SO₄. After filtration, the filtrate was concentrated under reduced pressure. The residue was purified by Prep-TLC (CH₂Cl₂/MeOH 20/1) followed by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min; Gradient: 21% B to 38% B in 10 min; Wave Length: 254 nm; RT1(min): 11.63) to afford (7R,14R)-1-(difluoromethoxy)-11-((1-(1-hydroxycyclopropane-1-carbonyl)pyrrolidin-3-yl)ethynyl)-6-(methyl-d3)-6,7-dihydro-7,14-methanobenzo[f]benzo[4,5]imidazo[1,2-a][1,4]diazocin-5(14H)-one (16 mg, 26%) as a white solid. MS ESI calculated for C₂₉H₂₃D₃F₂N₄O₄ [M+H]⁺, 536.21 found 536.20. ¹H NMR (400 MHz, Chloroform-d) δ 8.46 (d, J=8.1 Hz, 1H), 7.60 (d, J=8.4 Hz, 1H), 7.48-7.29 (m, 3H), 7.26-7.21 (m, 1H), 7.08-6.65 (m, 1H), 6.16 (d, J=7.2 Hz, 1H), 4.92 (d, J=7.0 Hz, 1H), 4.24-3.64 (m, 4H), 3.60-3.37 (m, 2H), 3.31-3.12 (m, 1H), 2.83 (d, J=13.6 Hz, 1H), 2.34-2.17 (m, 1H), 2.17-1.95 (m, 1H), 1.24-1.11 (m, 2H), 1.05-0.91 (m, 2H). ¹⁹F NMR (377 MHz, Chloroform-d) δ −79.99, −80.44, −81.31, −81.75.

### Example 204: (7R,14R)-1-(difluoromethoxy)-6-(methyl-d3)-11-((1-pivaloylpyrrolidin-3-yl)ethynyl)-6,7-dihydro-7,14-methanobenzo[f]benzo[4,5]imidazo[1,2-a][1,4]diazocin-5(14H)-one

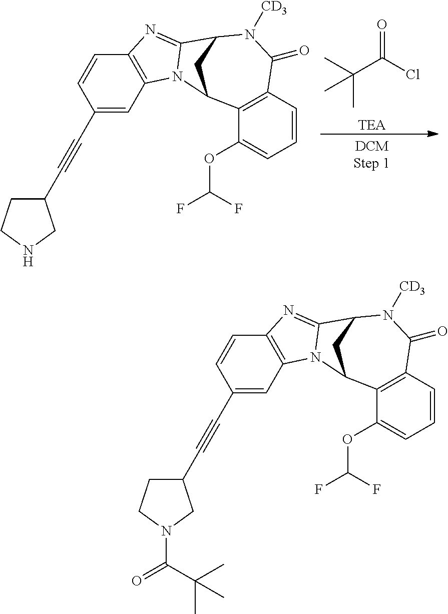

A solution of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (50 mg, 0.11 mmol), 2,2-dimethylpropanoyl chloride (16 mg, 0.13 mmol) and TEA (46 uL, 0.33 mmol) in DCM (2 mL) was stirred for 2 h at room temperature. The resulting mixture was concentrated under reduced pressure. The residue was purified by Prep-TLC (CH₂Cl₂/MeOH=25:1) followed by Prep-HPLC with the following conditions (Column: XBridge Prep OBD C18 Column 30*150 mm, 5 m; Mobile Phase A: Water (10 mmol/L NH₄HCO₃+0.1% NH₃·H₂O), Mobile Phase B: CH₃CN; Flow rate: 60 mL/min; Gradient: 30% B to 48% B in 10 min; Wave Length: 254 nm; RT1 (min): 11.05) to afford (7R,14R)-1-(difluoromethoxy)-6-(methyl-d3)-11-((1-pivaloylpyrrolidin-3-yl)ethynyl)-6,7-dihydro-7,14-methanobenzo[f]benzo[4,5]imidazo[1,2-a][1,4]diazocin-5(14H)-one (20 mg, 34%) as a white solid. MS ESI calculated for C₃₀H₂₇D₃F₂N₄O₃[M+H]⁺, 536.25. found 536.25. ¹H NMR (400 MHz, Chloroform-d) δ 8.52-8.46 (m, 1H), 7.65 (d, J=8.5 Hz, 1H), 7.53-7.48 (m, 1H), 7.48-7.41 (m, 1H), 7.38-7.34 (m, 1H), 7.33-7.28 (m, 1H), 6.86 (t, J=72.7 Hz, 1H), 6.25 (d, J=7.2 Hz, 1H), 5.07 (d, J=7.0 Hz, 1H), 3.96-3.72 (m, 2H), 3.70-3.54 (m, 2H), 3.54-3.41 (m, 1H), 3.26-3.11 (m, 1H), 2.89 (d, J=13.6 Hz, 1H), 2.10-1.86 (m, 2H), 1.28 (s, 9H). ¹⁹F NMR (377 MHz, Chloroform-d) δ −80.14, −80.58, −81.20, −81.64.

### Example 205: (7R,14R)-1-(difluoromethoxy)-11-((1-(1-hydroxycyclopropane-1-carbonyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one

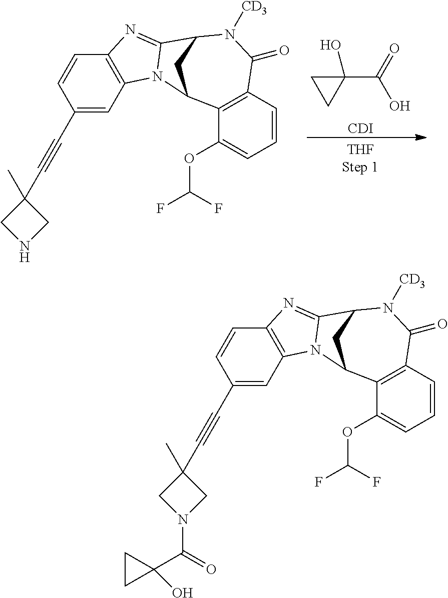

A mixture of 1-hydroxycyclopropane-1-carboxylic acid (27 mg, 0.266 mmol) and CDI (43 mg, 0.266 mmol) in THF (3 mL) was stirred for 10 min at room temperature. To the above mixture was added (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (60 mg, 0.133 mmol). The resulting mixture was stirred for additional 1 h at room temperature. The resulting mixture was concentrated under reduced pressure and purified by reversed-phase flash chromatography with the following conditions: column, C18 silica gel; mobile phase, CH₃CN in Water (10 mmol/L NH₄HCO₃), 40% to 60% gradient in 20 min. This resulted in (7R,14R)-1-(difluoromethoxy)-11-((1-(1-hydroxycyclopropane-1-carbonyl)-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one (25 mg, 34%) as a white solid. MS ESI calculated for C29H₂₃D₃F₂N₄O₄[M+H]⁺, 536.21. found 536.20. ¹H NMR (400 MHz, Chloroform-d) δ 8.55-8.47 (m, 1H), 7.67-7.57 (m, 2H), 7.49 (t, J=8.2 Hz, 1H), 7.43-7.37 (m, 1H), 7.37-7.30 (m, 1H), 6.90 (t, J=72.4 Hz, 1H), 6.45-6.34 (m, 1H), 5.50-5.37 (m, 1H), 4.80-4.57 (m, 1H), 4.50-4.12 (m, 2H), 4.09-3.87 (m, 1H), 3.82-3.57 (m, 1H), 2.96 (d, J=12.7 Hz, 1H), 1.67 (s, 3H), 1.41-1.30 (m, 2H), 1.29-1.22 (m, 1H), 1.07-0.97 (m, 2H). ¹⁹F NMR (377 MHz, Chloroform-d) δ −80.53, −80.97, −81.27, −81.72.

### Example 206: (7R,14R)-1-(difluoromethoxy)-11-((1-ethyl-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one

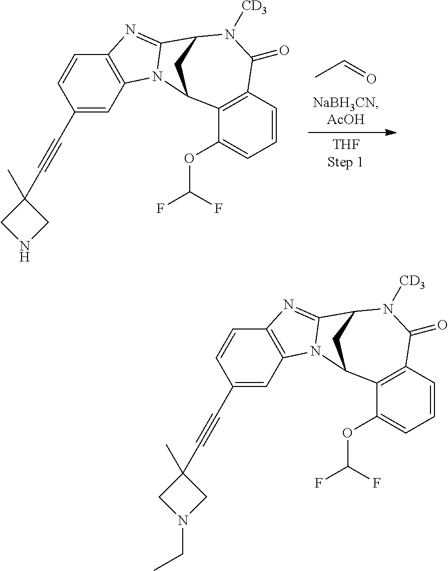

To a solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (60 mg, 0.133 mmol), AcOH (22 uL, 0.399 mmol) and acetaldehyde (17 mg, 0.399 mmol) in MeOH (3 mL) was added NaBH₃CN (25 mg, 0.399 mmol). The solution was stirred for 1 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 40% to 60% gradient in 20 min. This resulted in (7R,14R)-1-(difluoromethoxy)-11-((1-ethyl-3-methylazetidin-3-yl)ethynyl)-6-(methyl-d3)-6,7-dihydro-7,14-methanobenzo[f]benzo[4,5]imidazo[1,2-a][1,4]diazocin-5(14H)-one (6 mg, 10%) as a white solid. MS ESI calculated for C₂₇H₂₃D₃F₂N₄O₂ [M+H]⁺, 480.22. found 480.15. ¹H NMR (400 MHz, Chloroform-d) δ 8.53-8.43 (m, 1H), 7.66-7.59 (m, 1H), 7.52 (d, J=4.4 Hz, 1H), 7.43 (t, J=8.2 Hz, 1H), 7.34 (d, J=8.2 Hz, 1H), 7.29-7.27 (m, 1H), 6.88 (t, J=72.9 Hz, 1H), 6.27-6.16 (m, 1H), 4.94 (d, J=7.1 Hz, 1H), 3.91-3.56 (m, 4H), 3.49-3.37 (m, 1H), 3.05-2.78 (m, 3H), 1.77 (s, 3H), 1.32-1.13 (m, 3H). ¹⁹F NMR (377 MHz, Chloroform-d) δ −80.12, −80.58, −80.88, −81.35.

### Example 207: (7R,14R)-1-(difluoromethoxy)-11-(5-hydroxyhex-1-yn-1-yl)-6-(methyl-d3)-6,7-dihydro-7,14-methanobenzo[f]benzo[4,5]imidazo[1,2-a][1,4]diazocin-5(14H)-one

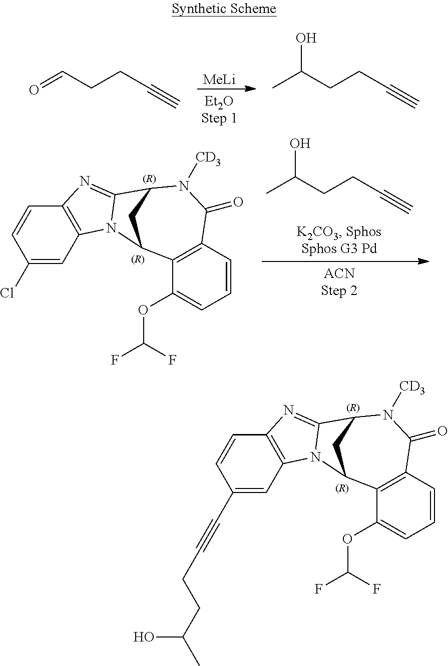

### Preparation 207A: hex-5-yn-2-ol

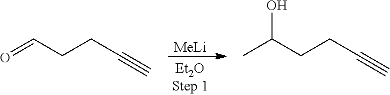

To a stirred solution of pent-4-yn-1-ol (1.00 g, 11.888 mmol) in diethyl ether (15 mL) was added methyllithium (11.42 mL, 18.270 mmol) dropwise at −78° C. under nitrogen atmosphere. The resulting mixture was stirred for 1 h at room temperature under nitrogen atmosphere. The reaction was quenched by the addition of sat. NH₄Cl (aq.) (50 mL) at 0° C. The resulting mixture was extracted with ether (3×30 mL). The combined organic layers were washed with brine (20 mL), dried over anhydrous Na₂SO₄. After filtration, the filtrate was concentrated under reduced pressure. This resulted in hex-5-yn-2-ol (1.16 g, crude) as a yellow oil. The crude resulting mixture was used in the next step directly without further purification. MS ESI calculated for C₆H₁₀O [M+H]⁺, 99.07. found N/A. ¹H NMR (400 MHz, Chloroform-d) δ 4.02-3.88 (m, 1H), 2.35-2.30 (m, 2H), 2.01-1.95 (m, 1H), 1.71-1.61 (m, 2H), 1.25-1.19 (m, 3H).

### Example 207: (7R,14R)-1-(difluoromethoxy)-11-(5-hydroxyhex-1-yn-1-yl)-6-(methyl-d3)-6,7-dihydro-7,14-methanobenzo[f]benzo[4,5]imidazo[1,2-a][1,4]diazocin-5(14H)-one

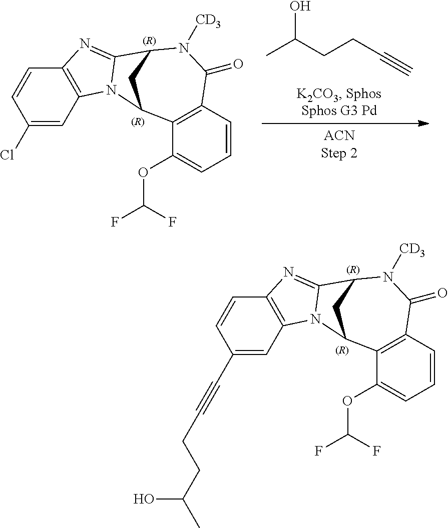

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (80 mg, 0.204 mmol), hex-5-yn-2-ol (100 mg, 1.020 mmol) and K₂CO₃(141 mg, 1.020 mmol) in CH₃CN (2.5 mL) were added Sphos (17 mg, 0.041 mmol) and SPhos Pd Gen.3 (32 mg, 0.041 mmol) in portions at room temperature. The resulting mixture was stirred for 3 h at 80° C. under nitrogen atmosphere. The resulting mixture was diluted with water (20 mL). The resulting mixture was extracted with EtOAc (3×20 mL). The combined organic layers were washed with brine (20 mL), dried over anhydrous Na₂SO₄. The residue was purified by silica gel column chromatography, eluted with CH₂Cl₂/MeOH (9/1) to followed by reversed-phase flash chromatography with the following conditions: column, C18 silica gel; mobile phase, CH₃CN in water (10 mmol/L NH₄HCO₃), 0% to 55% gradient in 20 min to afford (7R,14R)-1-(difluoromethoxy)-11-(5-hydroxyhex-1-yn-1-yl)-6-(methyl-d3)-6,7-dihydro-7,14-methanobenzo[f]benzo[4,5]imidazo[1,2-a][1,4]diazocin-5(14H)-one (36 mg, 22%) as a white solid. MS ESI calculated for C₂₅H₂₀D₃F₂N₃O₃[M+H]⁺, 455.19. found 455.05. ¹H NMR (400 MHz, Chloroform-d) δ 8.56-8.42 (m, 1H), 7.63 (d, J=8.5 Hz, 1H), 7.51 (d, J=1.4 Hz, 1H), 7.44 (t, J=8.2 Hz, 1H), 7.38-7.28 (m, 2H), 7.08-6.63 (m, 1H), 6.24 (d, J=7.0 Hz, 1H), 5.06 (d, J=6.8 Hz, 1H), 4.15-4.00 (m, 1H), 3.61-3.34 (m, 1H), 2.88 (d, J=13.6 Hz, 1H), 2.59-2.50 (m, 2H), 1.84-1.68 (m, 2H), 1.27 (d, J=6.2 Hz, 3H). ¹⁹F NMR (377 MHz, Chloroform-d) δ −80.05, −80.49, −81.18, −81.63.

### Example 208 and 209: (7R,14R)-1-(difluoromethoxy)-11-((R or S)-3-hydroxypent-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S or R)-3-hydroxypent-1-yn-1-yl)-6-(methyl-d3)-6,7-dihydro-7,14-methanobenzo[f]benzo[4,5]imidazo[1,2-a][1,4]diazocin-5(14H)-one

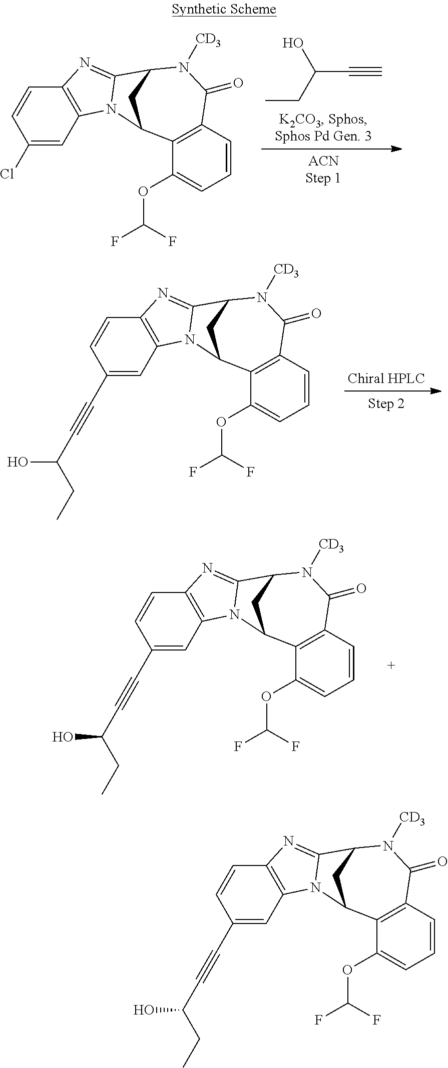

### Preparation 208A: (7R,14R)-1-(difluoromethoxy)-11-(3-hydroxypent-1-yn-1-yl)-6-methyl-d3)-6,7-dihydro-7,14-methanobenzo[f]benzo[4,5]imidazo[1,2-a][1,4]diazocin-5(14H)-one

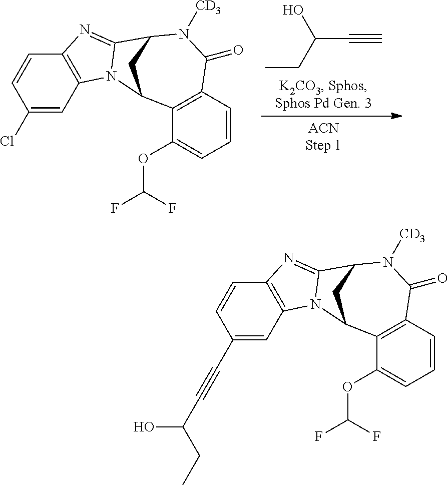

A mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (170 mg, 0.433 mmol), pent-1-yn-3-ol (182 mg, 2.165 mmol), SPhos Pd Gen.3 (67 mg, 0.087 mmol), SPhos (35 mg, 0.087 mmol) and K₂CO₃ (299 mg, 2.165 mmol) in ACN (8 mL) was stirred for 16 h at 80° C. under nitrogen atmosphere. The resulting mixture was concentrated under reduced pressure and purified by silica gel column chromatography, eluted with CH₂Cl₂/MeOH (10/1) followed by reversed-phase flash chromatography with the following conditions: column, C18 silica gel; mobile phase, CH₃CN in Water (10 mmol/L NH₄HCO₃), 30% to 50% gradient in 20 min. This resulted in (7R,14R)-1-(difluoromethoxy)-11-(3-hydroxypent-1-yn-1-yl)-6-(methyl-d3)-6,7-dihydro-7,14-methanobenzo[f]benzo[4,5]imidazo[1,2-a][1,4]diazocin-5(14H)-one (68 mg, 33%) as a white solid. MS ESI calculated for C₂₄H₁₈D₃F₂N₃O₃[M+H]⁺, 441.17. found 440.90.

### Example 208 and 209: (7R,14R)-1-(difluoromethoxy)-11-((R or S)-3-hydroxypent-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((S or R)-3-hydroxypent-1-yn-1-yl)-6-(methyl-d3)-6,7-dihydro-7,14-methanobenzo[f]benzo[4,5]imidazo[1,2-a][1,4]diazocin-5(14H)-one

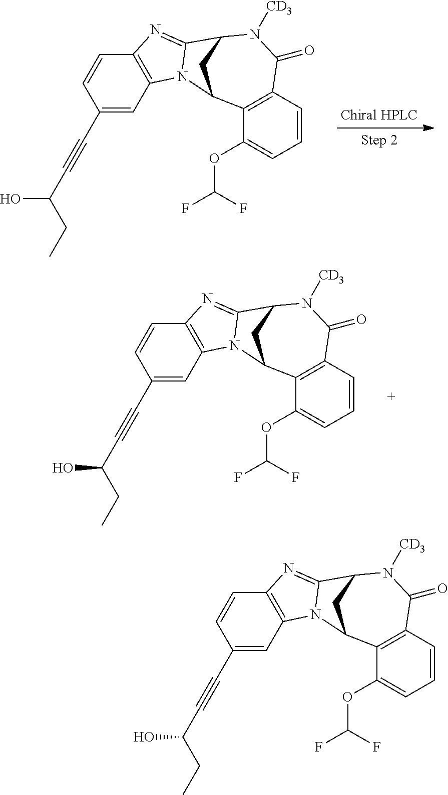 [SMILES: [2H]C([2H])([2H])N1C(=O)c2cccc(OC(F)F)c2[C@H]2C[C@@H]1c1nc3ccc(C#CC(O)CC)cc3n12 ; [2H]C([2H])([2H])N1C(=O)c2cccc(OC(F)F)c2[C@H]2C[C@@H]1c1nc3ccc(C#C[C@H](O)CC)cc3n12 ; [2H]C([2H])([2H])N1C(=O)c2cccc(OC(F)F)c2[C@H]2C[C@@H]1c1nc3ccc(C#C[C@@H](O)CC)cc3n12]

The mixture was purified by Chiral Prep-HPLC with the following conditions (Column: CHIRALPAK AD-3, 3.0*50 mm, 3 μm; Mobile Phase A: Hex (10 mM NH₃-MeOH), Mobile Phase B: EtOH; Flow rate: 40 mL/min; Gradient: isocratic 10; Wave Length: 226/206 nm; RT1(min): 29.952; RT2(min): 39.672). The first peak afforded (13 mg, 20%) as a white solid. MS ESI calculated for C₂₄H₁₈D₃F₂N₃O₃[M+H]⁺, 441.17. found 441.05. ¹H NMR (400 MHz, Chloroform-d) δ 8.51-8.45 (m, 1H), 7.62 (d, J=8.5 Hz, 1H), 7.53 (d, J=1.5 Hz, 1H), 7.42 (t, J=8.2 Hz, 1H), 7.34 (d, J=8.1 Hz, 1H), 7.29 (d, J=1.5 Hz, 1H), 7.06-6.66 (m, 1H), 6.22 (d, J=6.7 Hz, 1H), 5.01 (d, J=6.5 Hz, 1H), 4.58 (t, J=6.4 Hz, 1H), 3.55-3.40 (m, 1H), 2.87 (d, J=13.4 Hz, 1H), 1.94-1.77 (m, 2H), 1.09 (t, J=7.4 Hz, 3H). ¹⁹F NMR (377 MHz, Chloroform-d) δ −80.06, −80.50, −81.20, −81.65. The second peak afforded (16 mg, 23%) as a white solid. MS ESI calculated for C₂₄H₁₈D₃F₂N₃O₃[M+H]⁺, 441.17. found 441.05. ¹H NMR (400 MHz, Chloroform-d) δ 8.51-8.46 (m, 1H), 7.66-7.60 (m, 1H), 7.59-7.54 (m, 1H), 7.48-7.42 (m, 1H), 7.38-7.30 (m, 2H), 6.86 (t, J=72.7 Hz, 1H), 6.31-6.21 (m, 1H), 5.18-5.05 (m, 1H), 4.57 (t, J=6.4 Hz, 1H), 3.59-3.42 (m, 1H), 2.90 (d, J=13.2 Hz, 1H), 1.88-1.81 (m, 2H), 1.10 (t, J=7.4 Hz, 3H). ¹⁹F NMR (377 MHz, Chloroform-d) δ −80.15, −80.60, −81.23, −81.67.

### Example 210: (7R,14R)-11-((1-(2,2-difluorocyclopropane-1-carbonyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

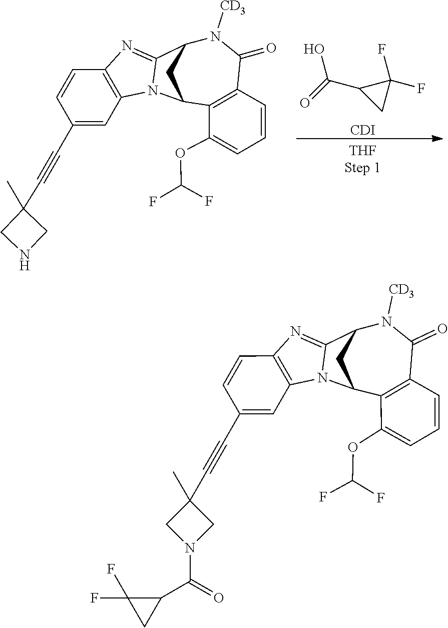

A mixture of 2,2-difluorocyclopropane-1-carboxylic acid (32 mg, 0.266 mmol) and CDI (43 mg, 0.266 mmol) in THF (2 mL) was stirred for 10 min at room temperature. To the above mixture was added (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (60 mg, 0.133 mmol). The resulting mixture was stirred for additional 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 30% to 50% gradient in 30 min. This resulted in (7R,14R)-11-((1-(2,2-difluorocyclopropane-1-carbonyl)-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (20 mg, 28%) as a white solid. MS ESI calculated for C₂₉H₂₁D₃F₄N₄O₃[M+H]⁺, 556.20. found 556.15. ¹H NMR (400 MHz, Chloroform-d) δ 8.49 (d, J=8.2 Hz, 1H), 7.66 (d, J=8.4 Hz, 1H), 7.53 (d, J=5.7 Hz, 1H), 7.45 (d, J=8.2 Hz, 1H), 7.41-7.30 (m, 2H), 6.86 (t, J=72.6 Hz, 1H), 6.29-6.19 (m, 1H), 5.12-4.96 (m, 1H), 4.48 (t, J=8.5 Hz, 1H), 4.40-4.24 (m, 1H), 4.12 (t, J=8.7 Hz, 1H), 4.03-3.89 (m, 1H), 3.57-3.41 (m, 1H), 2.89 (d, J=12.0 Hz, 1H), 2.36-2.12 (m, 2H), 1.74-1.65 (m, 1H), 1.69 (s, 3H).

### Example 211: (7R,14R)-11-((1-cyclopropyl-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

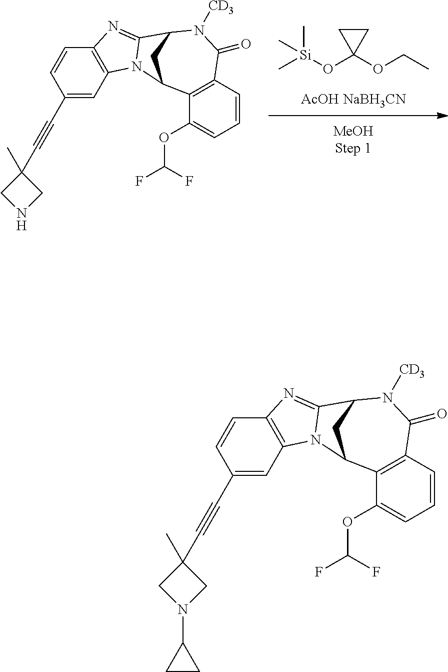

To a stirred solution of (7R,14R)-1-(difluoromethoxy)-6-(methyl-d3)-11-((3-methylazetidin-3-yl)ethynyl)-6,7-dihydro-7,14-methanobenzo[f]benzo[4,5]imidazo[1,2-a][1,4]diazocin-5(14H)-one (50 mg, 0.111 mmol), (1-ethoxycyclopropoxy)trimethylsilane (38 mg, 0.222 mmol) and acetic acid (12 uL, 0.222 mmol) in MeOH (2.50 mL) was added NaBH₃CN (10 mg, 0.167 mmol) in portions at room temperature. The resulting mixture was stirred for additional 2 h at 60° C. The resulting mixture was concentrated under vacuum and purified by reversed-phase flash chromatography with the following conditions: column, C18 silica gel; mobile phase, CH₃CN in Water (10 mmol/L NH₄HCO₃), 30% to 60% gradient in 30 min. This resulted in (7R,14R)-11-((1-cyclopropyl-3-methylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (14 mg, 26%) as a white solid. MS ESI calculated for C₂₈H₂₃D₃F₂N₄O₂[M+H]⁺, 492.22. found 492.25. ¹H NMR (400 MHz, Chloroform-d) δ 8.51-8.44 (m, 1H), 7.63-7.58 (m, 1H), 7.52-7.46 (m, 1H), 7.42 (t, J=8.2 Hz, 1H), 7.36-7.31 (m, 1H), 7.29-7.27 (m, 1H), 6.86 (t, J=72.8 Hz, 1H), 6.20 (d, J=7.3 Hz, 1H), 4.93 (d, J=7.1 Hz, 1H), 3.65-3.57 (m, 2H), 3.55-3.48 (m, 1H), 3.47-3.39 (m, 1H), 2.85 (d, J=13.6 Hz, 1H), 2.14-1.99 (m, 1H), 1.66-1.64 (m, 4H), 0.62-0.40 (m, 4H). ¹⁹F NMR (377 MHz, Chloroform-d) δ −79.97, −80.41, −81.23, −81.67.

### Example 212: (7R,14R)-1-(difluoromethoxy)-6-(methyl-d3)-11-((3-methyltetrahydrofuran-3-yl)ethynyl)-6,7-dihydro-7,14-methanobenzo[f]benzo[4,5]imidazo[1,2-a][1,4]diazocin-5(14H)-one

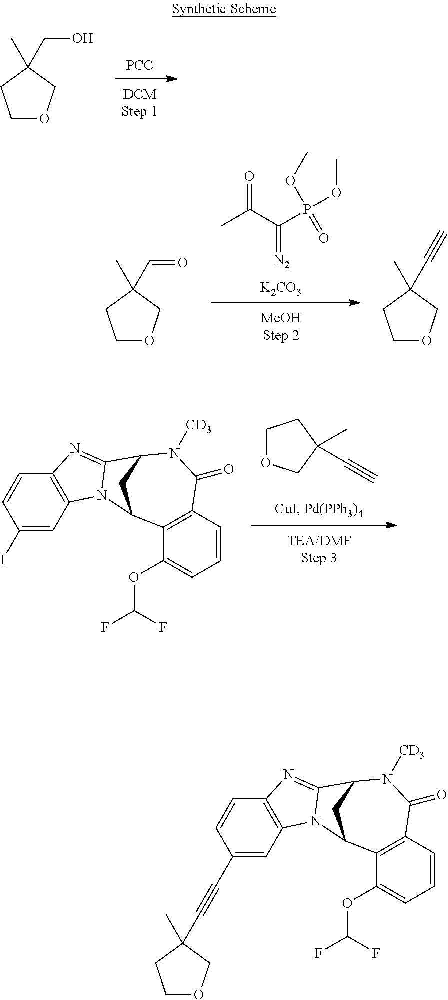

### Preparation 212A: 3-methyltetrahydrofuran-3-carbaldehyde

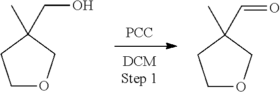

To a stirred solution of (3-methyltetrahydrofuran-3-yl)methanol (500 mg, 4.304 mmol) in DCM (8 mL) was added PCC (1.39 g, 6.456 mmol) at 0° C. The resulting mixture was stirred for 2 h at room temperature. The resulting mixture was diluted with Et₂O (20 mL). The resulting mixture was filtered, the filter cake was washed with Et₂O (3×5 mL). The filtrate was concentrated under reduced pressure to afford 3-methyltetrahydrofuran-3-carbaldehyde (500 mg, crude) as a yellow liquid. The crude product was used in the next step directly without further purification. MS ESI calculated for C₆H₁₀O₂ [M+H]⁺, 115.07. found N/A. ¹H NMR (400 MHz, Chloroform-d) δ 9.57 (s, 1H), 4.14-4.06 (m, 1H), 3.93-3.89 (m, 2H), 3.60-3.58 (m, 1H), 2.41-2.26 (m, 1H), 2.10-1.94 (m, 1H), 1.12 (s, 3H).

### Preparation 212B: 3-ethynyl-3-methyltetrahydrofuran

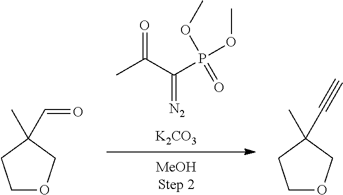

To a stirred solution of 3-methyltetrahydrofuran-3-carbaldehyde (500 mg, 4.380 mmol) and K₂CO₃ (1.82 g, 13.140 mmol) in MeOH (5 mL) were added dimethyl (1-diazo-2-oxopropyl)phosphonate (1.01 g, 5.256 mmol) dropwise at 0° C. under nitrogen atmosphere. The resulting mixture was stirred for 2 h at room temperature under nitrogen atmosphere. The reaction was quenched with water at room temperature. The resulting mixture was extracted with Et₂O (2×10 mL). The combined organic layers were washed with water (3×10 mL), dried over anhydrous Na₂SO₄, filtered. The organic layer was concentrated to afford 3-ethynyl-3-methyltetrahydrofuran (380 mg, crude) as a yellow liquid. The crude product was used in the next step directly without further purification. MS ESI calculated for C₇H₁₀O [M+H]⁺, 111.07, found N/A. ¹H NMR (400 MHz, Chloroform-d) δ 4.02-3.88 (m, 2H), 3.51-3.43 (m, 2H), 2.26-2.15 (m, 2H), 1.94-1.81 (m, 1H), 1.39-1.32 (m, 3H).

### Example 212: (7R,14R)-1-(difluoromethoxy)-6-(methyl-d3)-11-((3-methyltetrahydrofuran-3-yl)ethynyl)-6,7-dihydro-7,14-methanobenzo[f]benzo[4,5]imidazo[1,2-a][1,4]diazocin-5(14H)-one

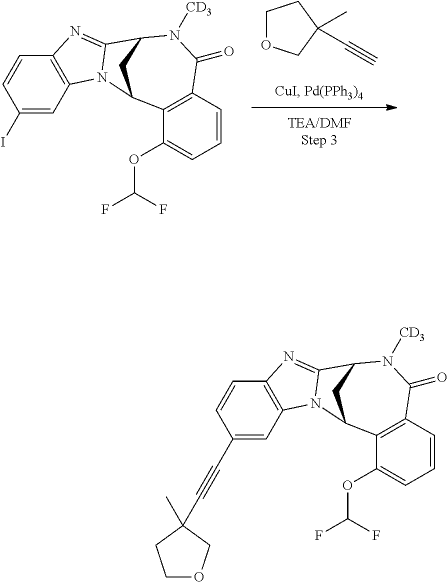

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (70 mg, 0.145 mmol) and 3-ethynyl-3-methyltetrahydrofuran (80 mg, 0.725 mmol) in TEA (0.5 mL) and DMF (0.5 mL) were added CuI (3 mg, 0.014 mmol) and Pd(PPh₃)₂Cl₂ (10 mg, 0.014 mmol) at room temperature under nitrogen atmosphere. The resulting mixture was stirred for 2 h at 80° C. under nitrogen atmosphere. The mixture was allowed to cool down to room temperature. The resulting mixture was filtered, the filter cake was washed with CH₂Cl₂/MeOH (3×10 mL, 10/1). The filtrate was concentrated under reduced pressure. The residue was purified by Prep-TLC (CH₂Cl₂/MeOH=10/1) followed by reversed-phase flash chromatography with the following conditions: column, C18 silica gel; mobile phase, CH₃CN in Water (10 mmol/L NH₄HCO₃), 30% to 50% gradient in 20 min to afford (7R,14R)-1-(difluoromethoxy)-6-(methyl-d3)-11-((3-methyltetrahydrofuran-3-yl)ethynyl)-6,7-dihydro-7,14-methanobenzo[f]benzo[4,5]imidazo[1,2-a][1,4]diazocin-5(14H)-one (33 mg, 49%) as a white solid. MS ESI calculated for C₂₆H₂₀D₃F₂N₃O₃[M+H]⁺, 467.19. found 467.20. ¹H NMR (400 MHz, Chloroform-d) δ 8.53-8.45 (m, 1H), 7.68-7.62 (m, 1H), 7.52 (s, 1H), 7.48-7.41 (m, 1H), 7.37-7.29 (m, 2H), 6.85 (t, J=72.7 Hz, 1H), 6.25 (d, J=7.0 Hz, 1H), 5.05 (d, J=7.3 Hz, 1H), 4.07-3.95 (m, 2H), 3.93 (d, J=8.1 Hz, 1H), 3.67 (d, J=8.0 Hz, 1H), 3.53-3.42 (m, 1H), 2.88 (d, J=13.5 Hz, 1H), 2.34-2.27 (m, 1H), 2.01-1.90 (m, 1H), 1.46 (s, 3H). ¹⁹F NMR (377 MHz, Chloroform-d) δ −80.11, −80.56, −81.32, −81.77.

### Example 213: N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)-2-hydroxy-2-methylpropanamide

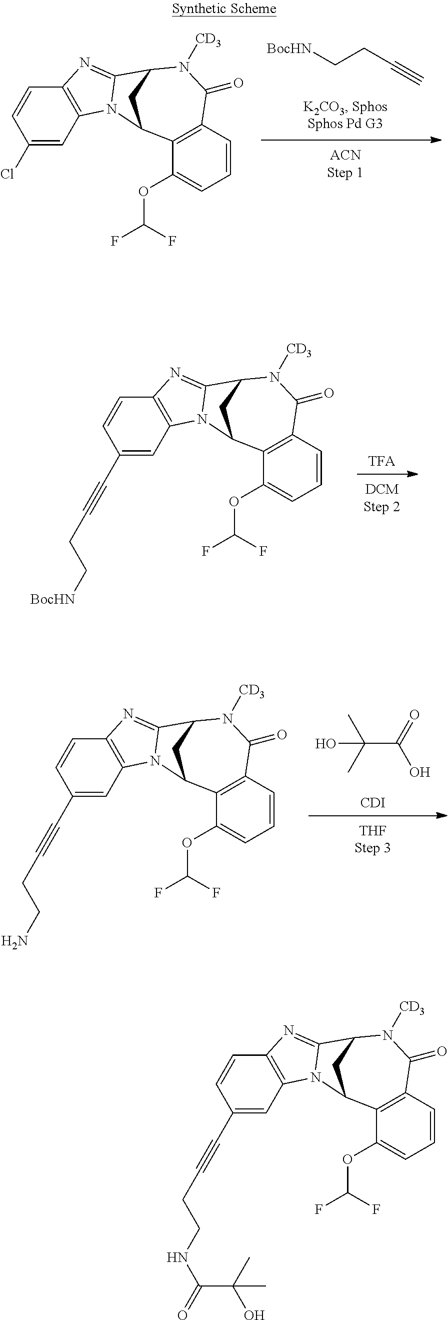

### Preparation 213A: tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)carbamate

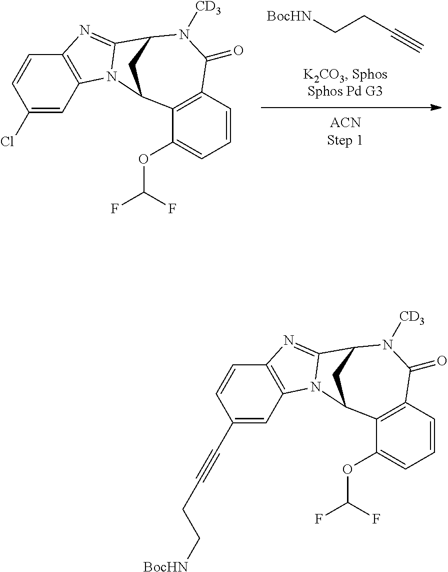 [SMILES: [2H]C([2H])([2H])N1C(=O)c2cccc(OC(F)F)c2[C@H]2C[C@@H]1c1nc3ccc(Cl)cc3n12 ; C#CCCNC(=O)OC(C)(C)C ; [2H]C([2H])([2H])N1C(=O)c2cccc(OC(F)F)c2[C@H]2C[C@@H]1c1nc3ccc(C#CCCNC(=O)OC(C)(C)C)cc3n12]

To a stirred mixture of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (200 mg, 0.509 mmol), SPhos (41 mg, 0.102 mmol), SPhos Pd Gen.3 (79 mg, 0.102 mmol) and K₂CO₃ (351 mg, 2.545 mmol) in ACN (4 mL) was added tert-butyl N-(but-3-yn-1-yl)carbamate (430 mg, 2.545 mmol) at room temperature. The resulting mixture was stirred for 16 h at 80° C. under nitrogen atmosphere. The resulting mixture was concentrated under reduced pressure. The residue was purified by silica gel column chromatography, eluted with CH₂Cl₂/MeOH (10/1) to followed by reversed-phase flash chromatography with the following conditions: column, C18 silica gel; mobile phase, CH₃CN in Water (10 mmol/L NH₄HCO₃), 10% to 42% gradient in 10 min to afford tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)carbamate (145 mg, 54%) as a white solid. MS ESI calculated for C₂₈H₂₅D₃F₂N₄O₄ [M+H]⁺, 526.23. found 526.25. ¹H NMR (400 MHz, Chloroform-d) δ 8.52-8.45 (m, 1H), 7.70-7.62 (m, 1H), 7.57-7.50 (m, 1H), 7.48-7.41 (m, 1H), 7.40-7.29 (m, 2H), 6.88 (t, J=72.6 Hz, 1H), 6.32-6.22 (m, 1H), 5.19-5.05 (m, 1H), 4.93-4.80 (m, 1H), 3.56-3.43 (m, 1H), 3.43-3.29 (m, 2H), 2.96-2.82 (m, 1H), 2.67-2.56 (m, 2H), 1.45 (s, 9H).

### Preparation 213B: (7R,14R)-11-(4-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

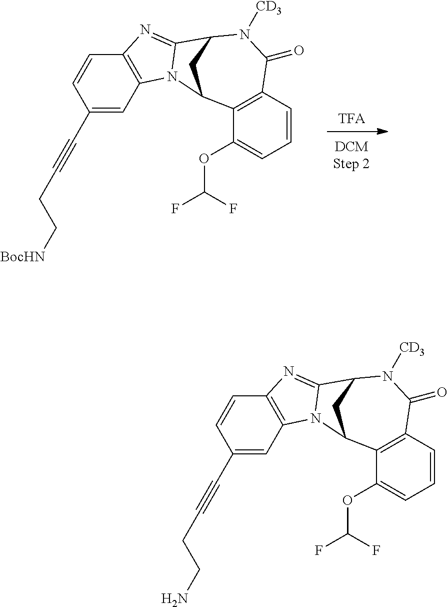 [SMILES: [2H]C([2H])([2H])N1C(=O)c2cccc(OC(F)F)c2[C@H]2C[C@@H]1c1nc3ccc(C#CCCNC(=O)OC(C)(C)C)cc3n12 ; "TFA" ; [2H]C([2H])([2H])N1C(=O)c2cccc(OC(F)F)c2[C@H]2C[C@@H]1c1nc3ccc(C#CCCN)cc3n12]

To a stirred solution of tert-butyl (4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)carbamate (140 mg, 0.266 mmol) in DCM (5 mL) was added TFA (1 mL) at room temperature. The resulting mixture was stirred for 2 h at room temperature. The resulting mixture was concentrated under reduced pressure. The residue was purified by reversed-phase flash chromatography with the following conditions: column, C18 silica gel; mobile phase, CH₃CN in Water (10 mmol/L NH₄HCO₃), 10% to 36% gradient in 10 min to afford (7R,14R)-11-(4-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 35%) as a white solid. MS ESI calculated for C₂₃H₁₇D₃F₂N₄O₂[M+H]⁺, 426.17. found 426.20.

### Example 213: N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)-2-hydroxy-2-methylpropanamide

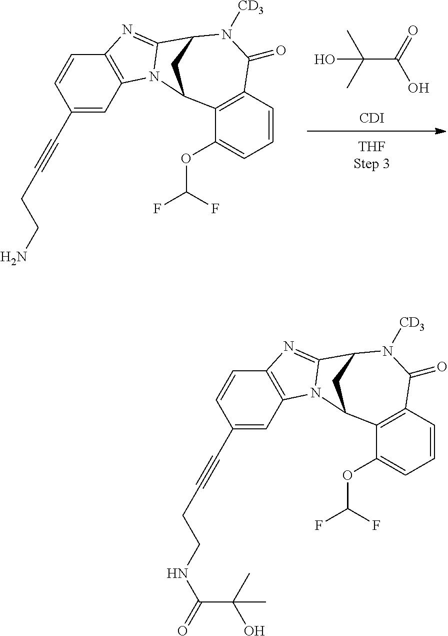

To a stirred solution of acetonate (14 mg, 0.141 mmol) in THF (2 mL) was added CDI (30 mg, 0.188 mmol) at room temperature. The resulting mixture was stirred for 10 min at room temperature. To the above mixture was added (7R,14R)-11-(4-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.094 mmol) in THF (0.1 mL) at room temperature. The resulting mixture was stirred for additional 2 h at room temperature. The resulting mixture was concentrated under reduced pressure. The crude product was purified by Prep-HPLC with the following conditions (Column: XBridge Prep Phenyl OBD Column 19*250 mm, 5m; Mobile Phase A: Water (10 mmol/L NH₄HCO₃), Mobile Phase B: CH₃CN; Flow rate: 60 mL/min mL/min; Gradient: 20% B to 35% B in 10 min; Wave Length: 254 nm; RT1(min): 11.52) to afford N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)-2-hydroxy-2-methylpropanamide (27 mg, 56%) as a white solid. MS ESI calculated for C27H₂₃D₃F₂N₄O₄[M+H]⁺, 512.21. found 512.26. ¹H NMR (400 MHz, Chloroform-d) 8.54-8.39 (m, 1H), 7.65-7.55 (m, 1H), 7.54-7.45 (m, 1H), 7.43-7.30 (m, 2H), 7.25-7.08 (m, 2H), 6.88 (t, J=72.7 Hz, 1H), 6.19 (d, J=7.2 Hz, 1H), 4.91 (d, J=6.8 Hz, 1H), 3.54-3.45 (m, 2H), 3.45-3.31 (m, 1H), 2.83 (d, J=13.5 Hz, 1H), 2.66 (t, J=6.4 Hz, 2H), 1.54-1.39 (m, 6H). ¹⁹F NMR (377 MHz, Chloroform-d) δ −79.85, −80.29, −81.10, −81.55.

### Example 214: N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)cyclopropanecarboxamide

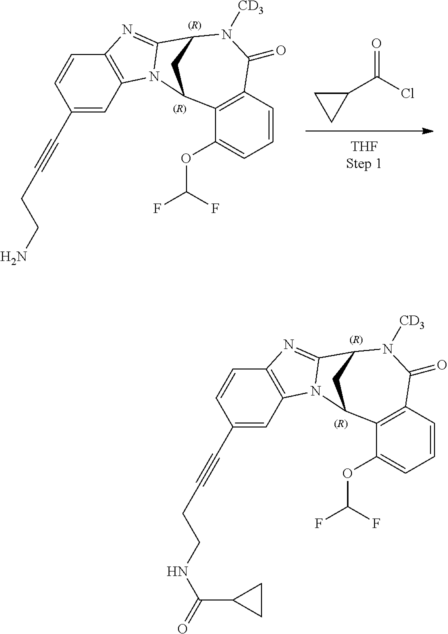

To a stirred solution of (7R,14R)-11-(4-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.094 mmol) in THF (2 mL) was added cyclopropanecarbonyl chloride (14 mg, 0.141 mmol) at 0° C. The resulting mixture was stirred for 2 h at room temperature. The resulting mixture was concentrated under reduced pressure and purified by Prep-HPLC with the following conditions (Column: Sunfire prep C18 column, 30*150 mm, 5m; Mobile Phase A: Water (0.1% FA), Mobile Phase B: ACN; Flow rate: 60 mL/min mL/min; Gradient: 23% B to 43% B in 10 min; Wave Length: 254 nm; RT1(min): 10.68) to afford N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)cyclopropanecarboxamide (12 mg, 26%) as a white solid. MS ESI calculated for C₂₇H₂₁D₃F₂N₄O₃ [M+H]⁺, 494.20. found 494.15. ¹H NMR (400 MHz, Chloroform-d) δ 8.58-8.39 (m, 1H), 7.71-7.39 (m, 3H), 7.38-7.28 (m, 2H), 6.86 (t, J=72.7 Hz, 1H), 6.34-5.91 (m, 2H), 5.14-4.84 (m, 1H), 3.65-3.38 (m, 3H), 2.93-2.79 (m, 1H), 2.76-2.58 (m, 2H), 1.43-1.31 (m, 1H), 1.05-0.92 (m, 2H), 0.83-0.69 (m, 2H). ¹⁹F NMR (377 MHz, Chloroform-d) δ −80.02, −80.47, −80.95, −81.40.

### Example 215: N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)pivalamide

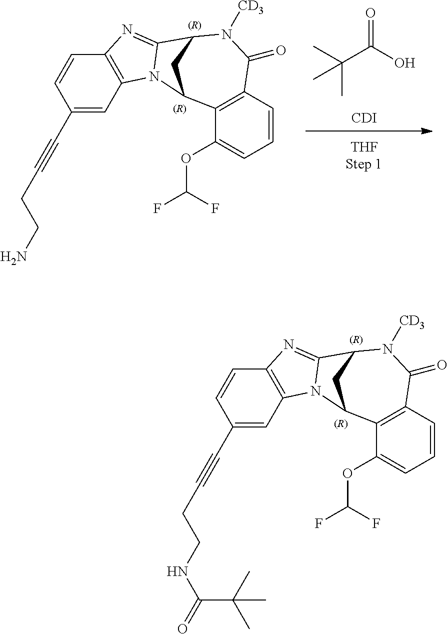

To a stirred solution of pivalic acid (14 mg, 0.141 mmol) in THF (0.2 mL) was CDI (30 mg, 0.188 mmol) added at room temperature. The resulting mixture was stirred for 10 min at room temperature. To the above mixture was added (7R,14R)-11-(4-aminobut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.094 mmol) at room temperature. The resulting mixture was stirred for additional 2 h at room temperature. The resulting mixture was concentrated under reduced pressure and purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: CH₃CN; Flow rate: 60 m/min m/min; Gradient: 30% B to 47% B in 10 min; Wave Length: 254 nm; RT1(min): 10.65) to afford N-(4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl)pivalamide (18 mg, 38%) as a white solid. MS ESI calculated for C₂₈H₂₅D₃F₂N₄O₃ [M+H]⁺, 510.23. found 510.20. ¹H NMR (400 MHz, Chloroform-d) δ 8.48 (d, J=8.1 Hz, 1H), 7.62 (d, J=8.4 Hz, 1H), 7.49 (s, 1H), 7.46-7.38 (m, 1H), 7.37-7.30 (m, 1H), 7.26-7.20 (m, 1H), 6.84 (t, J=72.8 Hz, 1H), 6.20 (d, J=7.2 Hz, 1H), 6.13-5.98 (m, 1H), 4.93 (d, J=7.1 Hz, 1H), 3.55-3.36 (m, 3H), 2.85 (d, J=13.4 Hz, 1H), 2.65 (t, J=6.3 Hz, 2H), 1.22 (s, 9H). ¹⁹F NMR (377 MHz, Chloroform-d) δ −79.94, −80.39, −80.79, −81.23.

### Example 216 and 217: (7R,14R)-11-((S or R)-3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((R or S)-3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

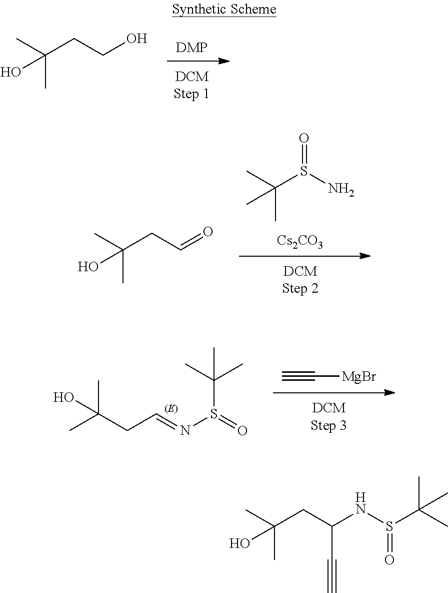

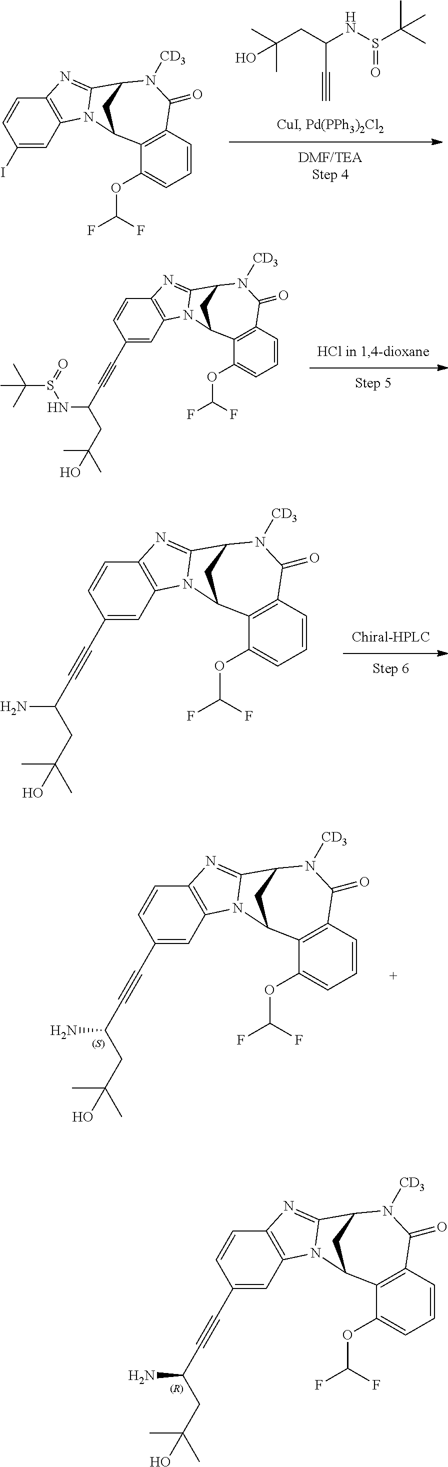

### Preparation 216A: 3-hydroxy-3-methylbutanal

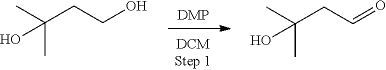

To a stirred solution of 2,4-dihydroxy-2-methylbutane (5.00 g, 48.008 mmol) in DCM (200 mL) was added DMP (30.54 g, 72.012 mmol) in portions at 0° C. The resulting mixture was stirred for overnight at room temperature. The resulting mixture was filtered, the filter cake was washed with DCM (3×50 mL). The filtrate was concentrated under reduced pressure. The residue was purified by silica gel column chromatography, eluted with PE/EA (1/1) to afford 3-hydroxy-3-methylbutanal (2.84 g, 58%) as a light yellow liquid. MS ESI calculated for C₅H₁₀O₂ [M+H]⁺, 103.07. found N/A.

### Preparation 216B: N-[(1Z)-3-hydroxy-3-methylbutylidene]-2-methylpropane-2-sulfinamide

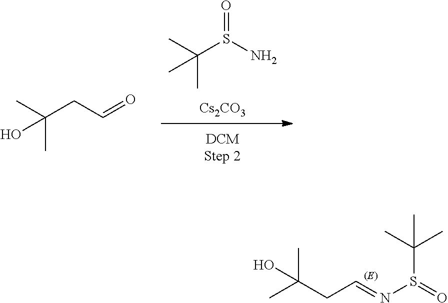

A solution of 3-hydroxy-3-methylbutanal (1.00 g, 9.791 mmol), tert-butanesulfinamide (1.42 g, 11.749 mmol) and Cs₂CO₃ (6.38 g, 19.582 mmol) in DCM (100 mL) was stirred for overnight at room temperature. The resulting mixture was filtered, the filter cake was washed with DCM (3×20 mL). The filtrate was concentrated under reduced pressure. The residue was purified by silica gel column chromatography, eluted with PE/EA (1/1) to afford N-[(1Z)-3-hydroxy-3-methylbutylidene]-2-methylpropane-2-sulfinamide (1.20 g, 60%) as a light yellow liquid. MS ESI calculated for C₉H₁₉NO₂S [M+H]⁺, 206.11. found 206.25. ¹H NMR (400 MHz, Chloroform-d) δ 8.19 (t, J=4.7 Hz, 1H), 2.74-2.67 (m, 2H), 1.36-1.33 (m, 6H), 1.22 (s, 9H).

### Preparation 216C: N-(5-hydroxy-5-methylhex-1-yn-3-yl)-2-methylpropane-2-sulfinamide

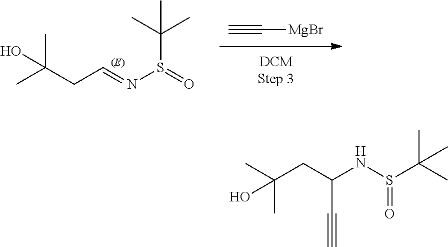

To a stirred solution of N-[(1Z)-3-hydroxy-3-methylbutylidene]-2-methylpropane-2-sulfinamide (500 mg, 2.435 mmol) in DCM (25 mL) was added bromo(ethynyl)magnesium (10 mL, 4.870 mmol) dropwise at −20° C. under nitrogen atmosphere. The resulting mixture was stirred for 3 h at room temperature under nitrogen atmosphere. The reaction was quenched by the addition of sat. NH₄Cl (aq.) (2 mL) at 0° C. The resulting mixture was extracted with CH₂Cl₂ (3×50 mL). The combined organic layers were washed with brine (3×100 mL), dried over anhydrous Na₂SO₄. After filtration, the filtrate was concentrated under reduced pressure to afford N-(5-hydroxy-5-methylhex-1-yn-3-yl)-2-methylpropane-2-sulfinamide (450 mg) as a yellow liquid. The crude product was used in the next step directly without further purification. MS ESI calculated for C₁₁H₂₁NO₂S [M+H]⁺, 232.13. found 232.00.

### Preparation 216D: N-(1-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-5-hydroxy-5-methylhex-1-yn-3-yl)-2-methylpropane-2-sulfinamide

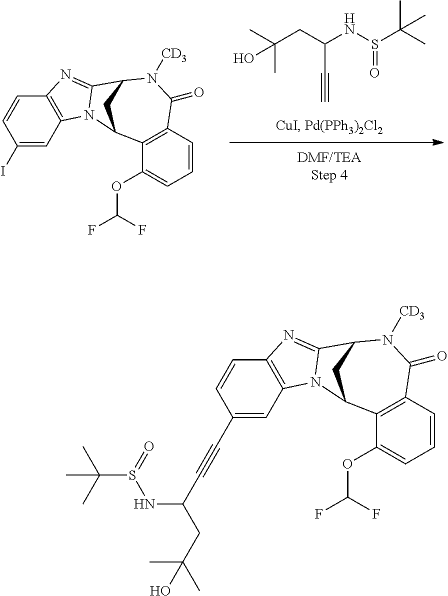 [SMILES: [2H]C([2H])([2H])N1C(=O)c2cccc(OC(F)F)c2[C@H]2C[C@@H]1c1nc3ccc(I)cc3n12 ; C#CC(CC(C)(C)O)NS(=O)C(C)(C)C ; [2H]C([2H])([2H])N1C(=O)c2cccc(OC(F)F)c2[C@H]2C[C@@H]1c1nc3ccc(C#CC(CC(C)(C)O)NS(=O)C(C)(C)C)cc3n12]

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (250 mg, 0.516 mmol) and N-(5-hydroxy-5-methylhex-1-yn-3-yl)-2-methylpropane-2-sulfinamide (597 mg, 2.580 mmol) in TEA (1 mL) and DMF (1 mL) were added Pd(PPh₃)2Cl₂ (36 mg, 0.052 mmol) and CuI (10 mg, 0.052 mmol) at room temperature. The resulting mixture was stirred for 1 h at 80° C. under nitrogen atmosphere. The resulting mixture was concentrated under reduced pressure and purified by silica gel column chromatography, eluted with CH₂Cl₂/MeOH (10/1) to afford N-(1-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-5-hydroxy-5-methylhex-1-yn-3-yl)-2-methylpropane-2-sulfinamide (230 mg, 76%) as a light yellow solid. MS ESI calculated for C₃₀H₃₁D₃F₂N₄O₄S [M+H]⁺, 588.25. found 588.25.

### Preparation 216E: (7R,14R)-11-(3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

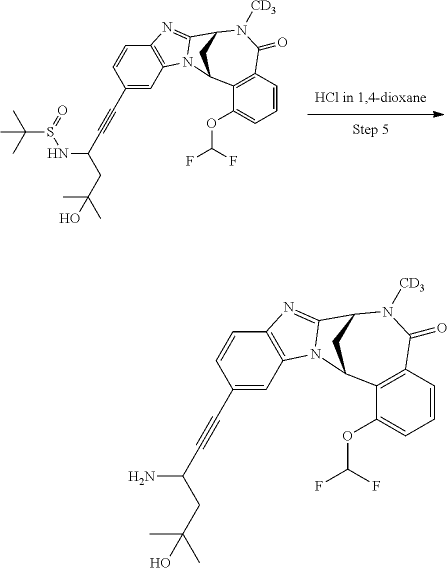 [SMILES: [2H]C([2H])([2H])N1C(=O)c2cccc(OC(F)F)c2[C@H]2C[C@@H]1c1nc3ccc(C#CC(CC(C)(C)O)NS(=O)C(C)(C)C)cc3n12 ; [2H]C([2H])([2H])N1C(=O)c2cccc(OC(F)F)c2[C@H]2C[C@@H]1c1nc3ccc(C#CC(N)CC(C)(C)O)cc3n12]

A solution of N-(1-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-5-hydroxy-5-methylhex-1-yn-3-yl)-2-methylpropane-2-sulfinamide (230 mg, 0.391 mmol) in HCl(gas) in 1,4-dioxane (10 mL) was stirred for 1 h at room temperature. The resulting mixture was concentrated under vacuum. The residue was purified by reversed-phase flash chromatography with the following conditions: column, C18 silica gel; mobile phase, CH₃CN in Water (10 mmol/L NH₄HCO₃), 30% to 60% gradient in 20 min. The fractions containing the desired product were collected at 45% B and concentrated under reduced pressure to afford (7R,14R)-11-(3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (90 mg, 48%) as a white solid. MS ESI calculated for C₂₆H₂₃D₃F₂N₄O₃ [M+H]⁺, 484.22. found 484.25.

### Example 216 and 217: (7R,14R)-11-((S or R)-3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one and (7R,14R)-11-((R or S)-3-amino-5-hydroxy-5-methylhex-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

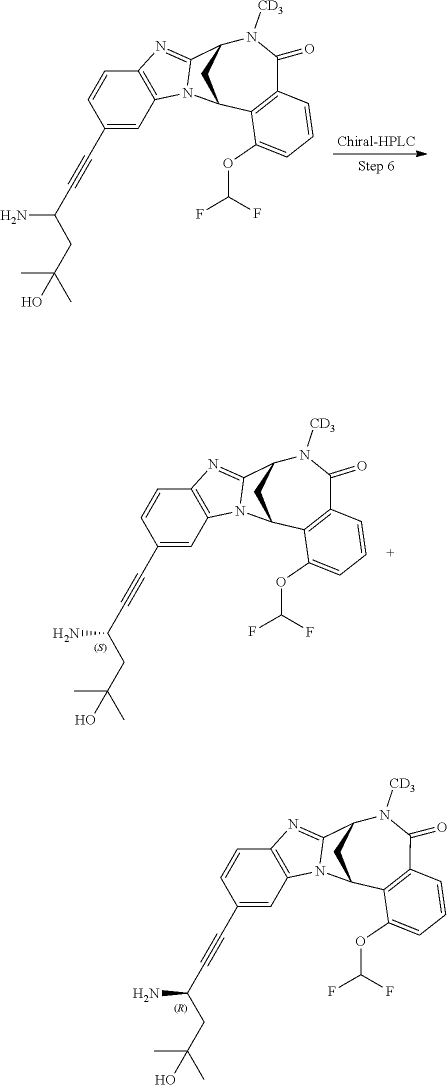

The mixture (80 mg) was purified by Prep-HPLC with the following conditions (Column: CHIRALPAK-IK, 3*25 mm, 5 μm; Mobile Phase A: Hex (10 mM NH₃-MeOH), Mobile Phase B: EtOH; Flow rate: 40 mL/min; Gradient: isocratic 30; Wave Length: 220/210 nm; RT1(min): 21.5; RT2(min): 27.7). The first peak afforded (9 mg, 11%) as a white solid. MS ESI calculated for C₂₆H₂₃D₃F₂N₄O₃[M+H]⁺, 484.22. found 484.15. ¹H NMR (400 MHz, DMSO-d) δ 8.31-8.17 (m, 1H), 7.84-7.57 (m, 2H), 7.56-7.31 (m, 4H), 6.27-6.14 (m, 1H), 5.25 (s, 1H), 5.18 (d, J=7.1 Hz, 1H), 3.56-3.37 (m, 5H), 2.85-2.71 (m, 1H), 1.98-1.85 (m, 1H), 1.37 (s, 3H), 1.23 (s, 3H). ¹⁹F NMR (377 MHz, DMSO-d₆) δ −81.04, −81.49, −82.33, −82.78. The second peak afforded (15 mg, 18%) as a white solid. MS ESI calculated for C₂₆H₂₃D₃F₂N₄O₃[M+H]⁺, 484.22. found 484.20. ¹H NMR (400 MHz, DMSO-d₆) δ 8.29-8.21 (m, 1H), 7.83-7.61 (m, 2H), 7.57-7.34 (m, 4H), 6.24 (d, J=7.1 Hz, 1H), 5.27 (s, 1H), 5.19 (d, J=7.1 Hz, 1H), 3.60-3.41 (m, 5H), 2.79 (d, J=13.7 Hz, 1H), 2.00-1.89 (m, 1H), 1.39 (s, 3H), 1.21 (s, 3H). ¹⁹F NMR (377 MHz, DMSO-d₆) δ −80.94, −81.39, −82.53, −82.98.

### Example 218: (7R,14R)-11-((1-cyclopropylpiperidin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

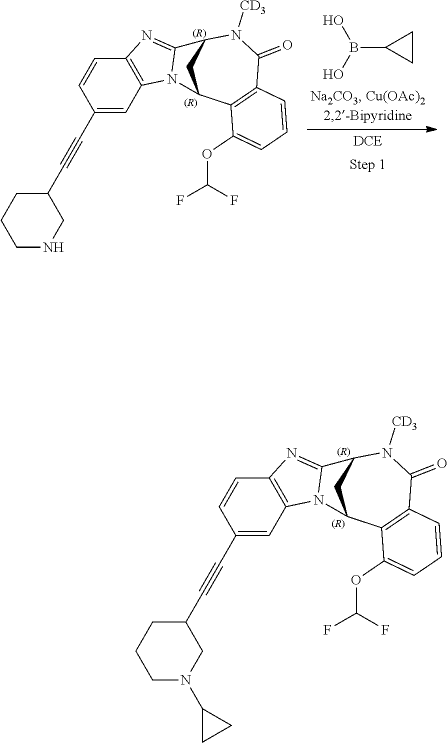

To a stirred mixture of (7R,14R)-1-(difluoromethoxy)-6-(methyl-d3)-11-(piperidin-3-ylethynyl)-6,7-dihydro-7,14-methanobenzo[f]benzo[4,5]imidazo[1,2-a][1,4]diazocin-5(14H)-one (50 mg, 0.107 mmol) and cyclopropylboronic acid (37 mg, 0.428 mmol) in DCE (2 mL) were added Na₂CO₃ (23 mg, 0.214 mmol), 2-(pyridin-2-yl)pyridine (17 mg, 0.111 mmol) and Cu(OAc)2 (20 mg, 0.111 mmol) at room temperature. The resulting mixture was stirred for 2 h at 70° C. under oxygen atmosphere. The resulting mixture was diluted with water (5 mL) and extracted with CH₂Cl₂ (3×10 mL). The combined organic layers were washed with brine (3×10 mL), dried over anhydrous Na₂SO₄. After filtration, the filtrate was concentrated under reduced pressure and purified by reverse phase flash with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 48% B to 65% B in 10 min; Wave Length: 254 nm; RT1(min): 9.67) to afford (7R,14R)-11-((1-cyclopropylpiperidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (11 mg, 19%) as a white solid. MS ESI calculated for C₂₉H₂₅D₃F₂N₄O₂[M+H]⁺, 506.24. found 506.20. ¹H NMR (400 MHz, DMSO-d₆) δ 8.31-8.24 (m, 1H), 7.81-7.39 (m, 5H), 7.17 (d, J=8.4 Hz, 1H), 6.21 (d, J=7.1 Hz, 1H), 5.21 (d, J=7.1 Hz, 1H), 3.58-3.44 (m, 1H), 3.07-2.97 (m, 1H), 2.80 (d, J=13.8 Hz, 2H), 2.70-2.58 (m, 1H), 2.35-2.17 (m, 2H), 1.90 (d, J=11.2 Hz, 1H), 1.69-1.58 (m, 2H), 1.52-1.30 (m, 2H), 0.47-0.37 (m, 2H), 0.37-0.23 (m, 2H). ¹⁹F NMR (377 MHz, DMSO-d₆) δ −82.17.

### Example 219: (7R,14R)-1-(difluoromethoxy)-6-(methyl-d3)-11-(5,5,5-trifluoro-3-hydroxypent-1-yn-1-yl)-6,7-dihydro-7,14-methanobenzo[f]benzo[4,5]imidazo[1,2-a][1,4]diazocin-5(14H)-one

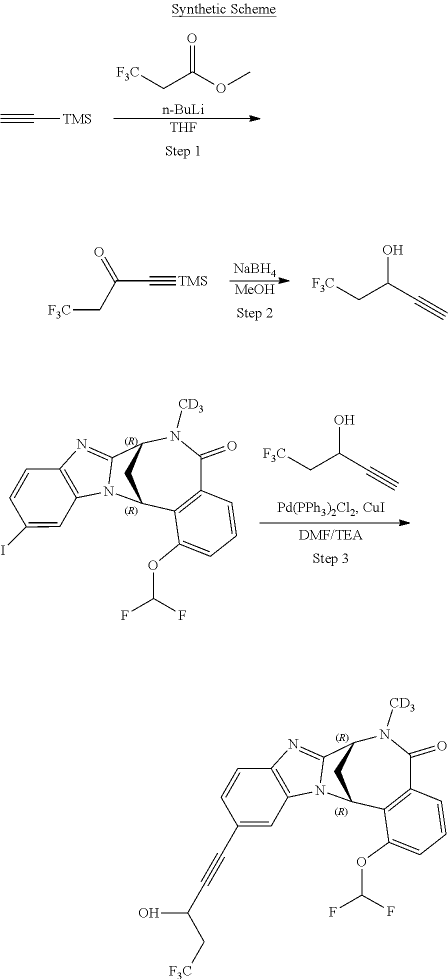

### Preparation 219A: 5,5,5-trifluoro-1-(trimethylsilyl)pent-1-yn-3-one

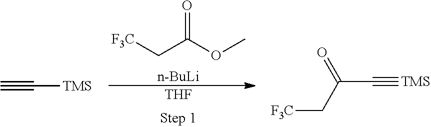

To a stirred solution of trimethylsilylacetylene (1.50 g, 15.272 mmol) in THF (10 mL) was added n-BuLi (6.7 mL, 16.799 mmol, 2.5M in hexane) dropwise at −78° C. and then the mixture was stirred for 1 h at −78° C. under nitrogen atmosphere. To above mixture was added methyl 3,3,3-trifluoropropanoate (2.60 g, 18.326 mmol) dropwise at −78° C. The resulting mixture was stirred for 4 h at room temperature under nitrogen atmosphere. The reaction was quenched by the addition of sat. NH₄Cl (aq.) (40 mL) at room temperature. The resulting mixture was extracted with EtOAc (3×20 mL). The combined organic layers were washed with brine (20 mL), dried over anhydrous Na₂SO₄. After filtration, the filtrate was concentrated under reduced pressure. The crude resulting mixture was used in the next step directly without further purification.

### Preparation 219B: 5,5,5-trifluoropent-1-yn-3-ol

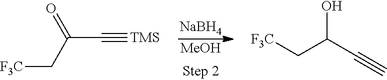

To a stirred solution of 5,5,5-trifluoro-1-(trimethylsilyl)pent-1-yn-3-one (2.00 g, 9.604 mmol) in MeOH (20 mL) was added NaBH₄ (726 mg, 19.208 mmol) in small portions at 0° C. under nitrogen atmosphere. The resulting mixture was stirred overnight at room temperature under nitrogen atmosphere. The reaction was quenched by the addition of water (80 mL) at room temperature. The resulting mixture was extracted with EtOAc (3×40 mL). The combined organic layers were washed with brine (50 mL), dried over anhydrous Na₂SO₄. After filtration, the filtrate was concentrated under reduced pressure to afford 5,5,5-trifluoropent-1-yn-3-ol (1.48 g) as a brown oil. The crude product was used in the next step directly without further purification. ¹⁹F NMR (377 MHz, Chloroform-d) δ −63.82.

### Example 219: (7R,14R)-1-(difluoromethoxy)-6-(methyl-d3)-11-(5,5,5-trifluoro-3-hydroxypent-1-yn-1-yl)-6,7-dihydro-7,14-methanobenzo[f]benzo[4,5]imidazo[1,2-a][1,4]diazocin-5(14H)-one

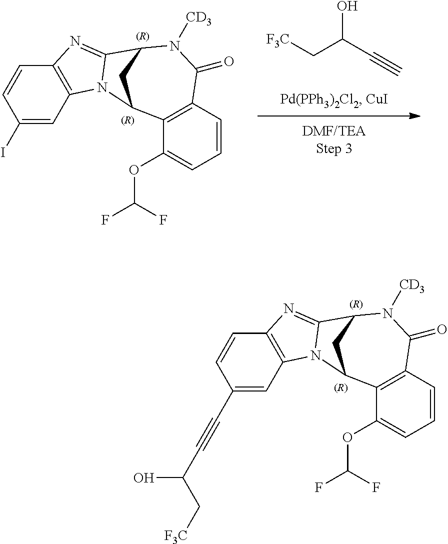

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (60 mg, 0.124 mmol) and 5,5,5-trifluoropent-1-yn-3-ol (85 mg, 0.620 mmol) in TEA (1 mL) and DMF (1 mL) were added CuI (4 mg, 0.025 mmol) and Pd(PPh₃)₂Cl₂ (8 mg, 0.012 mmol) at room temperature under nitrogen atmosphere. The resulting mixture was stirred for 3 h at 80° C. under nitrogen atmosphere. The resulting mixture was diluted with water (20 mL). The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in water (10 mmol/L NH₄HCO₃), 5% to 43% gradient in 20 min; detector, 254 nm to afford (7R,14R)-1-(difluoromethoxy)-6-(methyl-d3)-11-(5,5,5-trifluoro-3-hydroxypent-1-yn-1-yl)-6,7-dihydro-7,14-methanobenzo[f]benzo[4,5]imidazo[1,2-a][1,4]diazocin-5(14H)-one (11 mg, 5%) as a white solid. MS ESI calculated for C₂₄H₁₅D₃F₅N₃O₃[M+H]⁺, 495.15. found 495.00. ¹H NMR (400 MHz, Chloroform-d) δ 8.50-8.43 (m, 1H), 7.63 (d, J=8.4 Hz, 1H), 7.55-7.49 (m, 1H), 7.41 (t, J=8.2 Hz, 1H), 7.36-7.29 (m, 1H), 7.25-7.22 (m, 1H), 7.05-6.62 (m, 1H), 6.20 (d, J=7.3 Hz, 1H), 5.06-4.87 (m, 2H), 3.51-3.37 (m, 1H), 2.85 (d, J=13.6 Hz, 1H), 2.78-2.47 (m, 3H). ¹⁹F NMR (377 MHz, Chloroform-d) δ −63.62, −80.06, −80.50, −81.15, −81.59.

### Example 220: (7R,14R)-1-(difluoromethoxy)-11-(4-((S)-3-fluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one

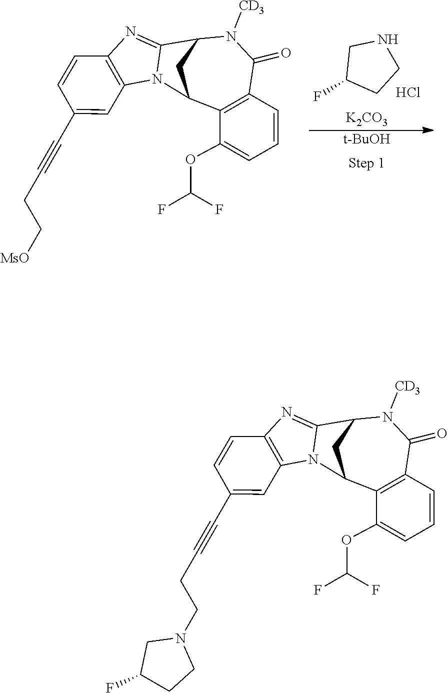

To a stirred solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (30 mg, 0.059 mmol) and (3S)-3-fluoropyrrolidine hydrochloride (14 mg, 0.118 mmol) in t-BuOH (5 mL) was added K₂CO₃ (24 mg, 0.177 mmol) at room temperature. The resulting mixture was stirred 24 h at 80° C. under nitrogen atmosphere. The resulting mixture was concentrated under reduced pressure and purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: MeOH; Flow rate: 60 mL/min; Gradient: 52% B to 69% B in 10 min; Wave Length: 254 nm; RT1(min): 11.12) to afford (7R,14R)-1-(difluoromethoxy)-11-(4-((S)-3-fluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one (6 mg, 15%) as a white solid. MS ESI calculated for C₂₇H₂₂D₃F₃N₄O₂[M+H]⁺, 498.21 found 498.30. ¹H NMR (400 MHz, Chloroform-d) δ 8.53-8.43 (m, 1H), 7.63-7.58 (m, 1H), 7.49 (t, J=1.0 Hz, 1H), 7.41 (t, J=8.2 Hz, 1H), 7.37-7.30 (m, 1H), 7.28-7.23 (m, 1H), 7.04-6.64 (m, 1H), 6.19 (d, J=7.3 Hz, 1H), 5.31-5.07 (m, 1H), 4.92 (d, J=7.1 Hz, 1H), 3.49-3.36 (m, 1H), 3.07-2.78 (m, 6H), 2.72-2.52 (m, 3H), 2.27-2.03 (m, 2H). ¹⁹F NMR (377 MHz, Chloroform-d) δ −79.89, −80.33, −81.13, −81.58, −168.15.

### Example 221 and 222: (7R,14R)-1-(difluoromethoxy)-11-((S or R)-3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R or S)-3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one

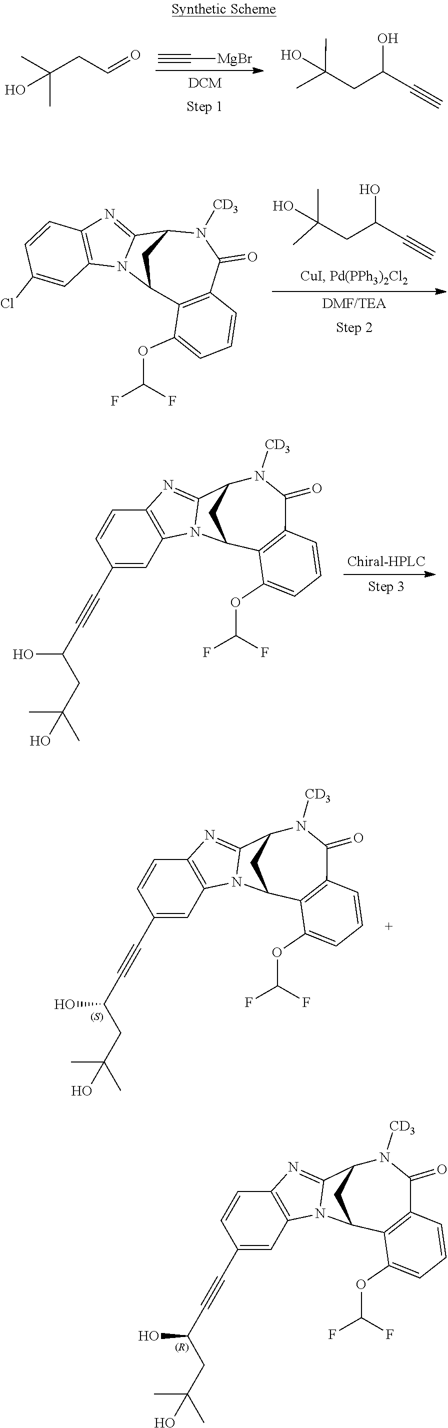

### Preparation 221A: 2-methylhex-5-yne-2,4-diol

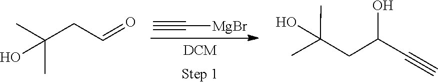

To a stirred solution of 3-hydroxy-3-methylbutanal (500 mg, 4.896 mmol) in DCM (60 mL) was added bromo(ethynyl)magnesium (19.58 mL, 9.792 mmol, 2 M in THF) dropwise at −20° C. under nitrogen atmosphere. The resulting mixture was stirred for 3 h at room temperature. The reaction was quenched with water and extracted with CH₂Cl₂ (3×50 mL). The combined organic layers were washed with brine (1×100 mL), dried over anhydrous Na₂SO₄. After filtration, the filtrate was concentrated under reduced pressure to afford 2-methylhex-5-yne-2,4-diol (300 mg, 48%) as a yellow oil. The crude product was used in the next step directly without further purification.

### Preparation 221B: (7R,14R)-1-(difluoromethoxy)-11-(3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one

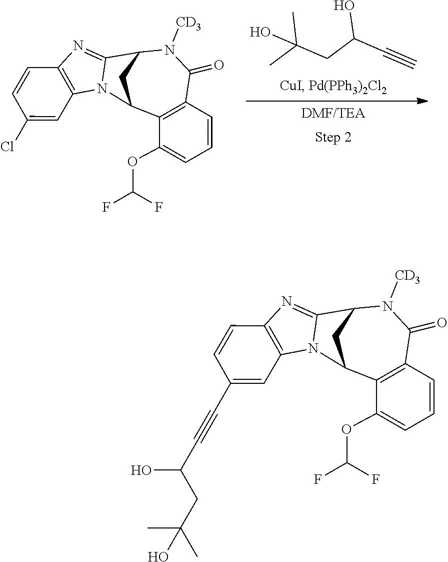

To a stirred solution of (7R,14R)-11-chloro-1-(difluoromethoxy)-6-(methyl-d3)-6,7-dihydro-7,14-methanobenzo[f]benzo[4,5]imidazo[1,2-a][1,4]diazocin-5(14H)-one (150 mg, 0.310 mmol) and 2-methylhex-5-yne-2,4-diol (199 mg, 1.550 mmol) in DMF (1 mL) and TEA (1 mL) were added Pd(PPh₃)₂Cl₂ (22 mg, 0.031 mmol) and CuI (6 mg, 0.031 mmol) at room temperature. The resulting mixture was stirred for 1 h at 80° C. under nitrogen atmosphere. The resulting mixture was concentrated under reduced pressure and purified by silica gel column chromatography, eluted with CH₂Cl₂/MeOH (10/1) to afford (7R,14R)-1-(difluoromethoxy)-11-(3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one (80 mg, 53%) as a white solid. MS ESI calculated for C₂₆H₂₂D₃F₂N₃O₄[M+H]⁺, 485.20. found 485.20.

### Example 221 and 222: (7R,14R)-1-(difluoromethoxy)-11-((S or R)-3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((R or S)-3,5-dihydroxy-5-methylhex-1-yn-1-yl)-6-(methyl-d3)-6,7-dihydro-7,14-methanobenzo[f]benzo[4,5]imidazo[1,2-a][1,4]diazocin-5(14H)-one

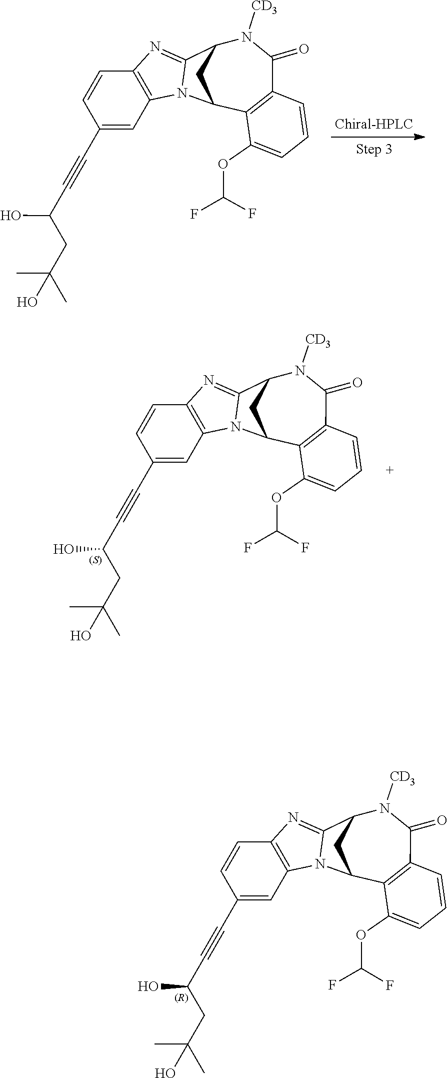

The mixture (80 mg) was purified by Chiral Prep-HPLC with the following conditions (Column: Chiral NQ(2)5u, 250*30 mm; Mobile Phase A: Hex (10 mM NH₃-MeOH), Mobile Phase B: IPA; Flow rate: 40 mL/min; Gradient: isocratic 20; Wave Length: 224/274 nm; RT1(min): 16.925; RT2(min): 21.941). The first peak afforded (24 mg, 36%) as a white solid. MS ESI calculated for C₂₆H₂₂D₃F₂N₃O₄[M+H]⁺, 485.20. found 485.10. ¹H NMR (400 MHz, DMSO-d) S 8.28 (d, J=7.4 Hz, 1H), 7.76-7.37 (m, 5H), 7.20 (d, J=8.5 Hz, 1H), 6.22 (d, J=7.1 Hz, 1H), 5.45 (d, J=5.2 Hz, 1H), 5.22 (d, J=6.9 Hz, 1H), 4.71-4.63 (m, 1H), 4.43 (d, J=2.3 Hz, 1H), 3.55-3.44 (m, 1H), 2.80 (d, J=13.8 Hz, 1H), 1.84 (d, J=6.2 Hz, 2H), 1.20 (s, 3H), 1.18 (s, 3H). ¹⁹F NMR (377 MHz, DMSO-d₆) δ −81.47, −81.91, −81.95, −82.40. The second peak afforded (23 mg, 32%) as a white solid. MS ESI calculated for C₂₆H₂₂D₃F₂N₃O₄[M+H]⁺, 485.20. found 485.10. ¹H NMR (400 MHz, DMSO-d₆) δ 8.31-8.25 (m, 1H), 7.77-7.37 (m, 5H), 7.20 (d, J=8.4 Hz, 1H), 6.22 (d, J=7.0 Hz, 1H), 5.47-5.43 (m, 1H), 5.22 (d, J=7.0 Hz, 1H), 4.71-4.63 (m, 1H), 4.43 (d, J=1.4 Hz, 1H), 3.57-3.44 (m, 1H), 2.80 (d, J=13.8 Hz, 1H), 1.91-1.79 (m, 2H), 1.20 (s, 3H), 1.19 (s, 3H). ¹⁹F NMR (377 MHz, DMSO-d₆) δ −81.45, −81.90, −81.96, −82.41.

### Example 223: (7R,14R)-11-((1-cyclopropylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

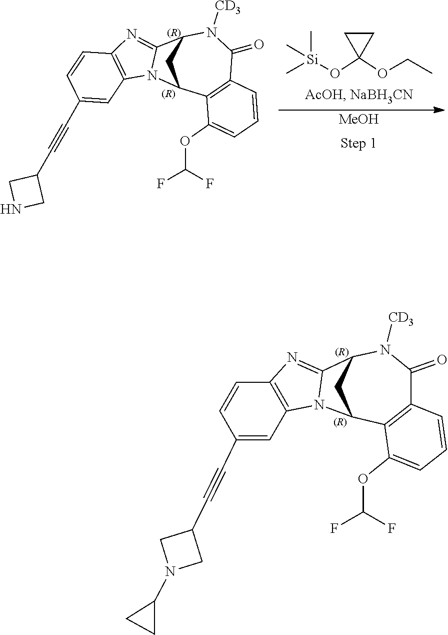

To a solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol), (1-ethoxycyclopropoxy)trimethylsilane (32 mg, 0.182 mmol) and AcOH (11 mg, 0.182 mmol) in MeOH (1 mL) was added NaBH₃CN (9 mg, 0.137 mmol). The solution was stirred for 1 h at room temperature. The resulting mixture was concentrated under reduced pressure and purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: CH₃CN; Flow rate: 60 mL/min; Gradient: 34% B to 51% B in 10 min; Wave Length: 254/220 nm; RT1(min): 10.73) to afford (7R,14R)-11-((1-cyclopropylazetidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (10 mg, 23%) as a white solid. MS ESI calculated for C₂₇H₂₁D₃F₂N₄O₂ [M+H]⁺, 478.21. found 478.15. ¹H NMR (400 MHz, Chloroform-d) δ 8.48 (d, J=8.2 Hz, 1H), 7.61 (d, J=8.5 Hz, 1H), 7.50 (s, 1H), 7.42 (t, J=8.2 Hz, 1H), 7.34 (d, J=8.1 Hz, 1H), 7.29 (d, J=1.4 Hz, 1H), 6.84 (t, J=72.7 Hz, 1H), 6.20 (d, J=7.2 Hz, 1H), 4.93 (d, J=7.1 Hz, 1H), 3.94-3.69 (m, 2H), 3.52-3.32 (m, 4H), 2.85 (d, J=13.6 Hz, 1H), 2.09-1.92 (m, 1H), 0.60-0.36 (m, 4H). ¹⁹F NMR (377 MHz, Chloroform-d) δ −79.88, −80.32, −81.07, −81.51.

### Example 224: (7R,14R)-1-(difluoromethoxy)-6-(methyl-d3)-11-((1-(oxetan-3-yl)azetidin-3-yl)ethynyl)-6,7-dihydro-7,14-methanobenzo[f]benzo[4,5]imidazo[1,2-a][1,4]diazocin-5(14H)-one

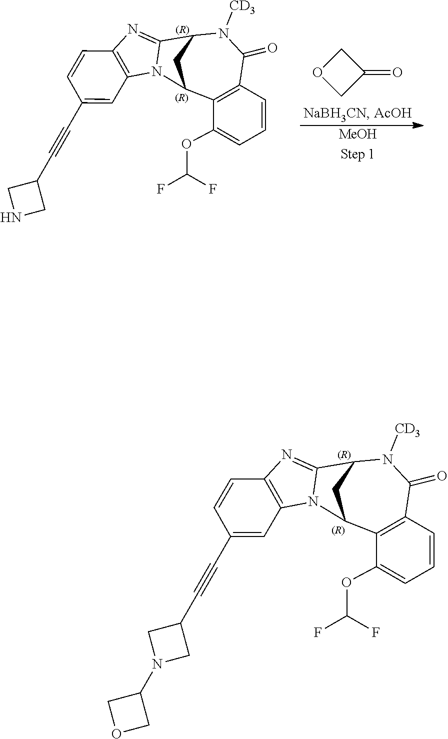

To a solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (30 mg, 0.069 mmol), AcOH (12 mg, 0.207 mmol) and 3-oxetanone (7 mg, 0.104 mmol) in MeOH (0.5 mL) was added NaBH₃CN (6 mg, 0.104 mmol). The reaction was stirred for 1 h at room temperature. The resulting mixture was purified by Prep-HPLC with the following conditions (Column: XBridge Prep OBD C18 Column 30*150 mm, 5m; Mobile Phase A: Water (10 mmol/L NH₄HCO₃+0.1% NH₃·H₂O), Mobile Phase B: CH₃CN; Flow rate: 60; Gradient: 17% B to 35% B in 10 min; Wave Length: 254/220 nm; RT1(min): 11.75) to afford (7R,14R)-1-(difluoromethoxy)-6-(methyl-d3)-11-((1-(oxetan-3-yl)azetidin-3-yl)ethynyl)-6,7-dihydro-7,14-methanobenzo[f]benzo[4,5]imidazo[1,2-a][1,4]diazocin-5(14H)-one (14 mg, 42%) as a white solid. MS ESI calculated for C₂₇H₂₁D₃F₂N₄O₃ [M+H]⁺, 494.20. found 494.15. ¹H NMR (400 MHz, Chloroform-d) δ 8.53-8.44 (m, 1H), 7.62 (d, J=8.5 Hz, 1H), 7.50 (d, J=1.5 Hz, 1H), 7.42 (t, J=8.2 Hz, 1H), 7.34 (d, J=8.1 Hz, 1H), 7.30-7.27 (m, 1H), 6.84 (t, J=72.8 Hz, 1H), 6.20 (d, J=7.2 Hz, 1H), 4.93 (d, J=7.1 Hz, 1H), 4.73 (t, J=6.8 Hz, 2H), 4.64-4.53 (m, 2H), 3.91-3.73 (m, 3H), 3.63-3.51 (m, 1H), 3.49-3.39 (m, 1H), 3.35 (t, J=7.2 Hz, 2H), 2.85 (d, J=13.6 Hz, 1H). ¹⁹F NMR (377 MHz, Chloroform-d) δ −79.92, −80.37, −80.98, −81.43.

### Example 225: (7R,14R)-11-((1-acetyl-3-methylpiperidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

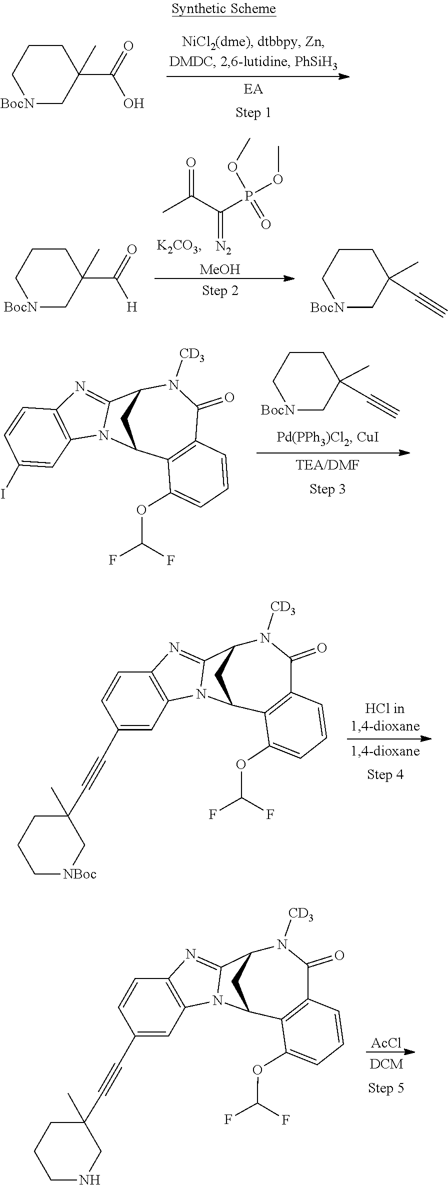

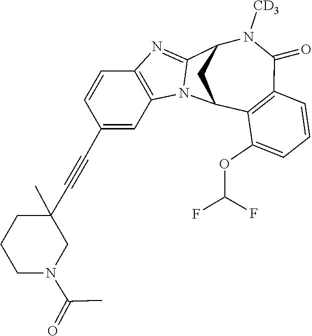 [SMILES: [2H]C([2H])([2H])N1C(=O)c2cccc(OC(F)F)c2[C@H]2C[C@@H]1c1nc3ccc(C#CC4(C)CCCN(C(C)=O)C4)cc3n12]

### Preparation 225A: tert-butyl 3-formyl-3-methylpiperidine-1-carboxylate

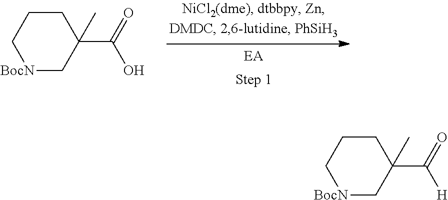 [SMILES: CC(C)(C)OC(=O)N1CCCC(C)(C(=O)O)C1 ; CC1(C=O)CCCN(C(=O)OC(C)(C)C)C1]

A mixture of NiCl₂(dme) (271 mg, 1.233 mmol) and 4-tert-butyl-2-(4-tert-butylpyridine-2-yl)pyridine (662 mg, 2.466 mmol) in EA (120 mL) was stirred for 10 min at room temperature under nitrogen atmosphere. To the above mixture were added 1-(tert-butoxycarbonyl)-3-methylpiperidine-3-carboxylic acid (3.0 g, 12.330 mmol), Zn (161 mg, 2.466 mmol), 2,6-lutidine (1.45 g, 13.563 mmol) and dimethyl dicarbonate (3.31 g, 24.660 mmol) in portions. The resulting mixture was stirred for additional 15 min at room temperature. Then phenylsilane (3.07 g, 28.359 mmol) was added. The resulting mixture was stirred for overnight at 75° C. under nitrogen atmosphere. The reaction was quenched with water. The mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (10/1) to afford tert-butyl 3-formyl-3-methylpiperidine-1-carboxylate (2.10 g, 75%) as a colorless oil. MS ESI calculated for C₁₂H₂₁NO₃ [M+H+CH₃CN]⁺, 269.19 found 269.25. ¹H NMR (400 MHz, Chloroform-d) δ 9.53 (s, 1H), 3.84 (d, J=13.3 Hz, 1H), 3.50-3.40 (m, 1H), 3.30-3.19 (m, 1H), 3.13 (d, J=13.3 Hz, 1H), 2.10-1.94 (m, 1H), 1.63-1.52 (m, 3H), 1.45 (s, 9H), 1.16 (s, 3H).

### Preparation 225B: tert-butyl 3-ethynyl-3-methylpiperidine-1-carboxylate

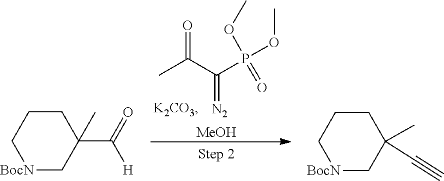

To a stirred mixture of tert-butyl 3-formyl-3-methylpiperidine-1-carboxylate (2.10 g, 9.239 mmol) and K₂CO₃ (2.55 g, 18.478 mmol) in MeOH (40 mL) was added dimethyl (1-diazo-2-oxopropyl)phosphonate (2.66 g, 13.859 mmol) at 0° C. The resulting mixture was stirred for 3 h at room temperature under nitrogen atmosphere. The reaction was quenched with water. The mixture was extracted with EtOAc (3×20 mL). The combined organic layers were washed with brine (3×20 mL), dried over anhydrous Na₂SO₄. After filtration, the filtrate was concentrated under reduced pressure. The residue was purified by reversed-phase flash chromatography with the following conditions: (column, C18 silica gel; mobile phase, CH₃CN in Water (0.1% FA), 10% to 50% gradient in 10 min; detector, 220 nm) to afford tert-butyl 3-ethynyl-3-methylpiperidine-1-carboxylate (320 mg, 16%) as a light yellow oil. MS ESI calculated for C₁₃H₂₁NO₂[M−t-Bu+H]⁺, 168.10. found 168.00. ¹H NMR (400 MHz, Chloroform-d) δ 3.86-3.71 (m, 2H), 3.00-2.89 (m, 1H), 2.87-2.76 (m, 1H), 2.08 (s, 1H), 1.89-1.74 (m, 2H), 1.60-1.43 (m, 2H), 1.44 (s, 9H), 1.20 (s, 3H).

### Preparation 225C: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylpiperidine-1-carboxylate

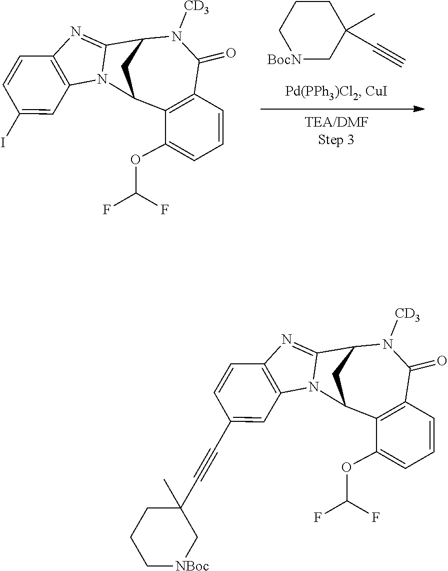 [SMILES: [2H]C([2H])([2H])N1C(=O)c2cccc(OC(F)F)c2[C@H]2C[C@@H]1c1nc3ccc(I)cc3n12 ; C#CC1(C)CCCN(C(=O)OC(C)(C)C)C1 ; [2H]C([2H])([2H])N1C(=O)c2cccc(OC(F)F)c2[C@H]2C[C@@H]1c1nc3ccc(C#CC4(C)CCCN(C(=O)OC(C)(C)C)C4)cc3n12]

To a stirred mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (110 mg, 0.227 mmol) and tert-butyl 3-ethynyl-3-methylpiperidine-1-carboxylate (76 mg, 0.341 mmol) in DMF (2 mL) and TEA (2 mL) were added Pd(PPh₃)₂Cl₂ (16 mg, 0.023 mmol) and CuI (4 mg, 0.023 mmol). The resulting mixture was stirred for 2 h at 80° C. under nitrogen atmosphere. The reaction was quenched with water. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 10% to 50% gradient in 10 min; detector, 254 nm to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylpiperidine-1-carboxylate (130 mg, 98%) as a light yellow solid. MS ESI calculated for C₃₂H₃₁D₃F₂N₄O₄ [M+H]⁺, 580.28. found 580.30.

### Preparation 225D: (7R,14R)-1-(difluoromethoxy)-6-(methyl-d3)-11-((3-methylpiperidin-3-yl)ethynyl)-6,7-dihydro-7,14-methanobenzo[f]benzo[4,5]imidazo[1,2-a][1,4]diazocin-5(14H)-one

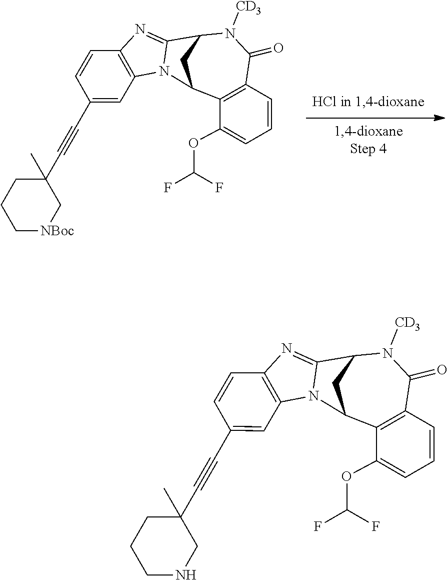 [SMILES: [2H]C([2H])([2H])N1C(=O)c2cccc(OC(F)F)c2[C@H]2C[C@@H]1c1nc3ccc(C#CC4(C)CCCN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4(C)CCCNC4)cc3n12]

To a stirred mixture of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-methylpiperidine-1-carboxylate (130 mg, 0.224 mmol) in 1,4-dioxane (1 mL) was added HCl(gas) in 1,4-dioxane (1 mL, 4 M) slowly. The resulting mixture was stirred for 2 h at room temperature under nitrogen atmosphere. The mixture was concentrated under vacuum, and neutralized to pH=7 with saturated NaHCO₃(aq.). The resulting mixture was extracted with CH₂Cl₂ (3×10 mL). The combined organic layers were dried over anhydrous Na₂SO₄. After filtration, the filtrate was concentrated under reduced pressure. The crude product was used in the next step directly without further purification. MS ESI calculated for C₂₇H₂₃D₃F₂N₄O₂[M+H]⁺, 480.23. found 480.20.

### Example 225: (7R,14R)-11-((1-acetyl-3-methylpiperidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

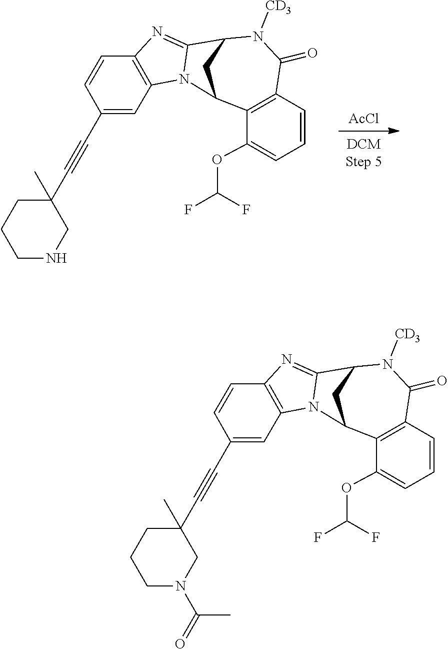 [SMILES: "AcCl" ; [2H]C([2H])([2H])N1C(=O)c2cccc(OC(F)F)c2[C@H]2C[C@@H]1c1nc3ccc(C#CC4(C)CCCNC4)cc3n12 ; [2H]C([2H])([2H])N1C(=O)c2cccc(OC(F)F)c2[C@H]2C[C@@H]1c1nc3ccc(C#CC4(C)CCCN(C(C)=O)C4)cc3n12]

To a stirred mixture of (7R,14R)-1-(difluoromethoxy)-6-(methyl-d3)-11-((3-methylpiperidin-3-yl)ethynyl)-6,7-dihydro-7,14-methanobenzo[f]benzo[4,5]imidazo[1,2-a][1,4]diazocin-5(14H)-one (110 mg, 0.229 mmol) in DCM (2 mL) was added acetyl chloride (21 mg, 0.275 mmol). The resulting mixture was stirred for 1 h at room temperature under nitrogen atmosphere. The reaction was quenched with water and extracted with DCM (3×10 mL). The combined organic layers were dried over anhydrous Na₂SO₄. After filtration, the filtrate was concentrated under reduced pressure. The residue was purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: ACN; Flow rate: 60 mL/min; Gradient: 26% B to 46% B in 10 min; Wave Length: 254 nm; RT1(min): 10.28) to afford (7R,14R)-11-((1-acetyl-3-methylpiperidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (42 mg, 35%) as an off-white solid. MS ESI calculated for C₂₉H₂₅D₃F₂N₄O₃[M+H]⁺, 522.24. found 522.35. ¹H NMR (400 MHz, DMSO-d₆) δ 8.33-8.21 (m, 1H), 7.82-7.36 (m, 5H), 7.19-7.08 (m, 1H), 6.28-6.17 (m, 1H), 5.26-5.15 (m, 1H), 4.38-4.10 (m, 1H), 3.87-3.61 (m, 1H), 3.55-3.42 (m, 1H), 3.17-3.00 (m, 1H), 2.86-2.76 (m, 1H), 2.63-2.52 (m, 1H), 2.08-1.97 (m, 3H), 1.91-1.47 (m, 4H), 1.24 (d, J=13.7 Hz, 3H). ¹⁹F NMR (377 MHz, DMSO-d₆) δ −81.58, −82.03, −82.12, −82.18, −82.20, −82.21, −82.33, −82.78.

### Example 226: (7R,14R)-1-(difluoromethoxy)-11-(4-(3,3-difluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one

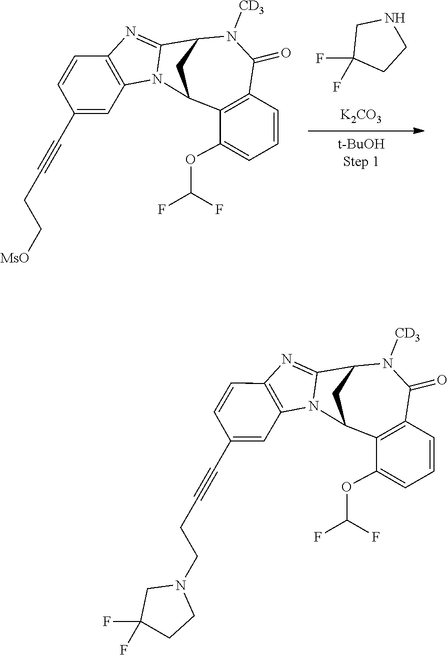

A solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (40 mg, 0.08 mmol), 3,3-difluoropyrrolidine (17 mg, 0.16 mmol) and K₂CO₃ (33 mg, 0.24 mmol) in t-BuOH (1 mL) was stirred for 16 h at 80° C. The resulting mixture was concentrated under reduced pressure. The residue was purified by Prep-TLC (100% EA) followed by Prep-HPLC with the following conditions (Column: Sunfire prep C18 column 30*150 mm, 5 m; Mobile Phase A: Water (0.1% FA), Mobile Phase B: ACN; Flow rate: 60 mL/min; Gradient: 11% B to 31% B in 10 min; Wave Length: 254 nm) to afford (7R,14R)-1-(difluoromethoxy)-11-(4-(3,3-difluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one (9 mg, 23%) as a brown solid. MS ESI calculated for C₂₇H₂₁D₃F₄N₄O₂ [M+H]⁺, 516.20. found 516.15. ¹H NMR (400 MHz, Chloroform-d) δ 8.55-8.42 (m, 1H), 7.62 (d, J=8.0 Hz, 1H), 7.50 (s, 1H), 7.46-7.38 (m, 1H), 7.34 (d, J=8.1 Hz, 1H), 7.29-7.27 (m, 1H), 7.06-6.62 (m, 1H), 6.21 (d, J=7.1 Hz, 1H), 4.95 (d, J=6.9 Hz, 1H), 3.54-3.35 (m, 1H), 3.28-3.10 (m, 2H), 3.09-2.98 (m, 2H), 2.97-2.81 (m, 3H), 2.72 (q, J=7.2, 6.0 Hz, 2H), 2.44-2.30 (m, 2H). ¹⁹F NMR (377 MHz, Chloroform-d) δ −80.02, −80.46, −81.13, −81.57, −93.27.

### Example 227: (7R,14R)-1-(difluoromethoxy)-11-(4-((R)-3-fluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one

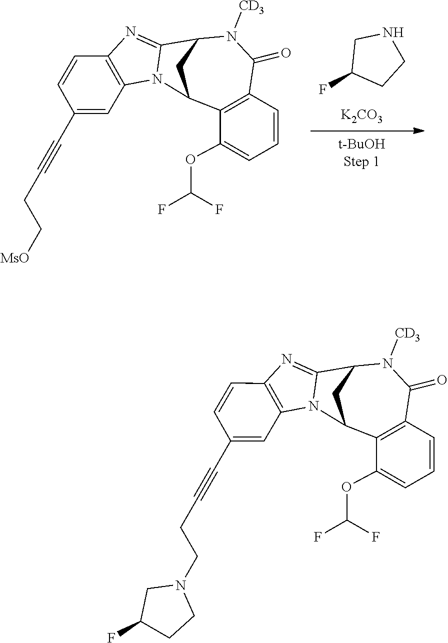

To a stirred solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (30 mg, 0.059 mmol) and (3R)-3-fluoropyrrolidine (10 mg, 0.119 mmol) in t-BuOH (5 mL) were added K₂CO₃ (24 mg, 0.177 mmol) at room temperature. The resulting mixture was stirred 24 h at 80° C. The reaction was concentrated under reduced pressure and purified by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min; Gradient: 30% B to 47% B in 10 min; Wave Length: 254 nm to afford (7R,14R)-1-(difluoromethoxy)-11-(4-((R)-3-fluoropyrrolidin-1-yl)but-1-yn-1-yl)-6-(methyl-d3)-6,7-dihydro-7,14-methanobenzo[f]benzo[4,5]imidazo[1,2-a][1,4]diazocin-5(14H)-one (10 mg, 36%) as a white solid. MS ESI calculated for C₂₇H₂₂D₃F₃N₄O₂[M+H]⁺, 498.21. found 498.30. ¹H NMR (400 MHz, Chloroform-d) δ 8.51-8.44 (m, 1H), 7.63-7.57 (m, 1H), 7.50 (d, J=1.5 Hz, 1H), 7.45-7.38 (m, 1H), 7.34 (d, J=8.1 Hz, 1H), 7.25 (q, J=1.5 Hz, 1H), 7.05-6.63 (m, 1H), 6.20 (d, J=7.3 Hz, 1H), 5.23 (d, J=54.7 Hz, 1H), 4.95-4.86 (m, 1H), 3.47-3.35 (m, 1H), 3.22-2.62 (m, 9H), 2.32-2.07 (m, 2H). ¹⁹F NMR (377 MHz, Chloroform-d) δ −79.91, −80.36, −81.12, −81.57.

### Example 228: (7R,14R)-1-(difluoromethoxy)-11-(4-(3-fluoroazetidin-1-yl)but-1-yn-1-yl)-6-(methyl-d3)-6,7-dihydro-7,14-methanobenzo[f]benzo[4,5]imidazo[1,2-a][1,4]diazocin-5(14H)-one

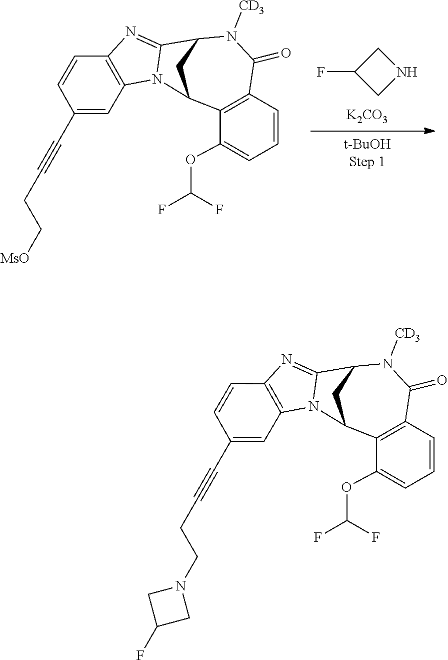

To a stirred solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (50 mg, 0.099 mmol) and 3-fluoroazetidine hydrochloride (22 mg, 0.198 mmol) in t-BuOH (4 mL) was added K₂CO₃ (41 mg, 0.297 mmol) at room temperature. The resulting mixture was stirred 24 h at 80° C. under nitrogen atmosphere. The reaction was concentrated under reduced pressure and purified by Prep-HPLC with the following conditions (Column: Sunfire prep C18 column, 30*150 mm, 5m; Mobile Phase A: Water (0.1% FA), Mobile Phase B: ACN; Flow rate: 60 mL/min; Gradient: 2% B to 22% B in 10 min; Wave Length: 254 nm; RT1(min): 11.03) to afford (7R,14R)-1-(difluoromethoxy)-11-(4-(3-fluoroazetidin-1-yl)but-1-yn-1-yl)-6-(methyl-d3)-6,7-dihydro-7,14-methanobenzo[f]benzo[4,5]imidazo[1,2-a][1,4]diazocin-5(14H)-one (6 mg, 13%) as a white solid. MS ESI calculated for C₂₆H₂₀D₃F₃N₄O₂[M+H]⁺, 484.20 found 484.25. ¹H NMR (400 MHz, Chloroform-d) δ 8.52-8.45 (m, 1H), 7.60 (d, J=8.4 Hz, 1H), 7.49 (d, J=1.5 Hz, 1H), 7.42 (t, J=8.2 Hz, 1H), 7.33 (d, J=8.1 Hz, 1H), 7.26-7.24 (m, 1H), 7.04-6.61 (m, 1H), 6.20 (d, J=7.2 Hz, 1H), 5.27-5.02 (m, 1H), 4.93 (d, J=7.1 Hz, 1H), 3.87-3.75 (m, 2H), 3.49-3.36 (m, 1H), 3.33-3.16 (m, 2H), 2.89-2.73 (m, 3H), 2.52 (t, J=7.0 Hz, 2H). ¹⁹F NMR (377 MHz, Chloroform-d) δ −79.92, −80.37, −81.08, −81.52, −178.00.

### Example 229: (7R,14R)-1-(difluoromethoxy)-6-(methyl-d3)-11-(3-morpholinoprop-1-yn-1-yl)-6,7-dihydro-7,14-methanobenzo[f]benzo[4,5]imidazo[1,2-a][1,4]diazocin-5(14H)-one

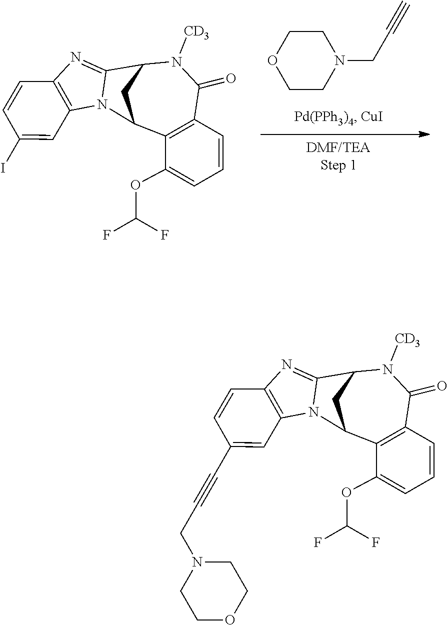

To a stirred solution of ((7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (80 mg, 0.165 mmol), CuI (6 mg, 0.033 mmol) and Pd(PPh₃)₄(19 mg, 0.017 mmol) in DMF (0.6 mL) and TEA (0.6 mL) was added 4-(prop-2-yn-1-yl)morpholine (31 mg, 0.247 mmol) at room temperature. The resulting mixture was stirred overnight at 80° C. under nitrogen atmosphere. The resulting mixture was filtered and purified by reversed-phase flash chromatography with the following conditions: column, C18 silica gel; mobile phase, CH₃CN in Water (0.1% TFA), 40% to 50% gradient in 10 min; detector, 254 nm. This resulted in (7R,14R)-1-(difluoromethoxy)-6-(methyl-d3)-11-(3-morpholinoprop-1-yn-1-yl)-6,7-dihydro-7,14-methanobenzo[f]benzo[4,5]imidazo[1,2-a][1,4]diazocin-5(14H)-one (21 mg, 27%) as a light yellow solid. MS ESI calculated for C₂₆H₂₁D₃F₂N₄O [M+H]⁺, 482.20 found 482.20. ¹H NMR (400 MHz, Chloroform-d) δ 8.55-8.39 (m, 1H), 7.64 (d, J=8.4 Hz, 1H), 7.62-7.56 (m, 1H), 7.43 (t, J=8.2 Hz, 1H), 7.38-7.30 (m, 2H), 6.87 (t, J=72.7 Hz, 1H), 6.21 (d, J=7.2 Hz, 1H), 4.94 (d, J=7.1 Hz, 1H), 4.08-3.81 (m, 4H), 3.78-3.58 (m, 2H), 3.49-3.40 (m, 1H), 3.07-2.62 (m, 5H). ¹⁹F NMR (377 MHz, Chloroform-d) δ −80.14, −80.60, −81.03, −81.48.

### Example 230: (7R,14R)-1-(difluoromethoxy)-11-(3-(3,3-difluoropyrrolidin-1-yl)prop-1-yn-1-yl)-6-(methyl-d3)-6,7-dihydro-7,14-methanobenzo[f]benzo[4,5]imidazo[1,2-a][1,4]diazocin-5(14H)-one

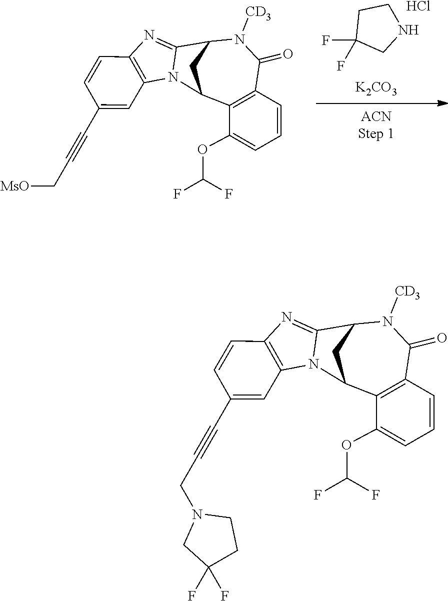

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl methanesulfonate (40 mg, 0.082 mmol) and K₂CO₃ (34 mg, 0.246 mmol) in ACN (1 mL) was added 3,3-difluoropyrrolidine hydrochloride (14 mg, 0.098 mmol) at room temperature. The resulting mixture was stirred at 50° C. for 2 h. The resulting mixture was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: MeOH; Flow rate: 60 mL/min; Gradient: 50% B to 68% B in 10 min; Wave Length: 254 nm; RT1(min): 9.95) to afford (7R,14R)-1-(difluoromethoxy)-11-(3-(3,3-difluoropyrrolidin-1-yl)prop-1-yn-1-yl)-6-(methyl-d3)-6,7-dihydro-7,14-methanobenzo[f]benzo[4,5]imidazo[1,2-a][1,4]diazocin-5(14H)-one (10 mg, 25%) as a white solid. MS ESI calculated for C26H₁₉D₃F₄N₄O₂ [M+H]⁺, 502.19 found 502.15. ¹H NMR (400 MHz, Chloroform-d) δ 8.55-8.43 (m, 1H), 7.64 (d, J=8.4 Hz, 1H), 7.56 (s, 1H), 7.42 (t, J=8.2 Hz, 1H), 7.36-7.28 (m, 2H), 6.84 (t, J=72.7 Hz, 1H), 6.22 (d, J=7.2 Hz, 1H), 4.94 (d, J=7.1 Hz, 1H), 3.75 (s, 2H), 3.55-3.39 (m, 1H), 3.22 (t, J=13.0 Hz, 2H), 3.14-3.00 (m, 2H), 2.86 (d, J=13.6 Hz, 1H), 2.49-2.28 (m, 2H). ¹⁹F NMR (377 MHz, Chloroform-d) δ −80.11, −80.55, −81.11, −81.55, −92.95.

### Example 231: (7R,14R)-11-(3-(3,3-difluoroazetidin-1-yl)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

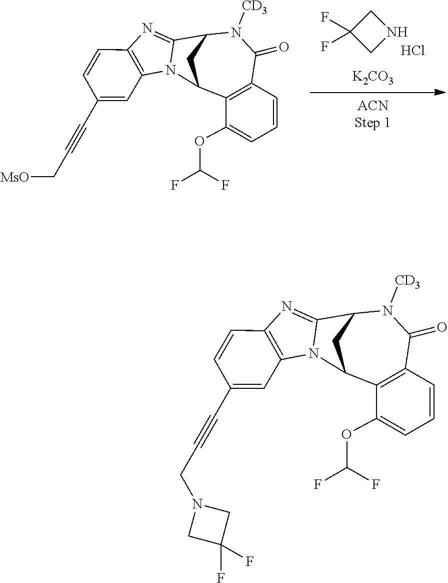

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl methanesulfonate (40 mg, 0.082 mmol) and K₂CO₃ (34 mg, 0.246 mmol) in ACN (1 mL) was added 3,3-difluoroazetidine hydrochloride (13 mg, 0.098 mmol) at room temperature. The resulting mixture was stirred at 50° C. for 2 h. The resulting mixture was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: MeOH; Flow rate: 60 mL/min; Gradient: 45% B to 65% B in 10 min; Wave Length: 254 nm; RT1(min): 10.37) to afford (7R,14R)-11-(3-(3,3-difluoroazetidin-1-yl)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (9 mg, 22%) as a white solid. MS ESI calculated for C₂₅H₁₇D₃F₄N₄O₂ [M+H]⁺, 488.17 found 488.10. ¹H NMR (400 MHz, Chloroform-d) δ 8.56-8.45 (m, 1H), 7.65 (d, J=8.4 Hz, 1H), 7.59 (d, J=1.5 Hz, 1H), 7.43 (d, J=8.2 Hz, 1H), 7.39-7.31 (m, 2H), 6.83 (t, J=72.2 Hz, 1H), 6.23 (d, J=7.2 Hz, 1H), 4.95 (d, J=7.2 Hz, 1H), 3.87 (t, J=12.0 Hz, 4H), 3.75 (s, 2H), 3.52-3.42 (m, 1H), 2.87 (d, J=13.6 Hz, 1H). ¹⁹F NMR (377 MHz, Chloroform-d) δ −80.03, −80.48, −81.23, −81.68, −99.43.

### Example 232: (7R,14R)-1-(difluoromethoxy)-11-(3-(3-fluoroazetidin-1-yl)prop-1-yn-1-yl)-6-(methyl-d3)-6,7-dihydro-7,14-methanobenzo[f]benzo[4,5]imidazo[1,2-a][1,4]diazocin-5(14H)-one

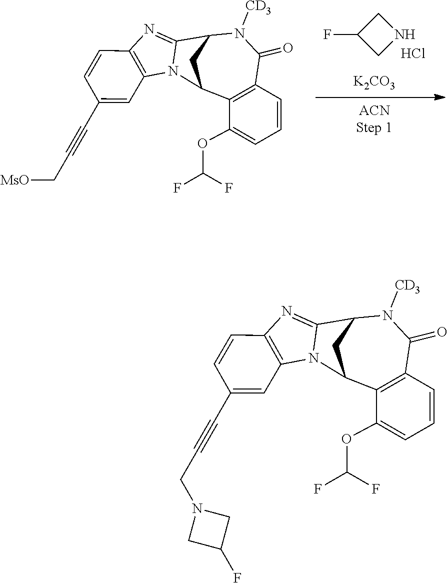

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)prop-2-yn-1-yl methanesulfonate (40 mg, 0.082 mmol) and K₂CO₃ (34 mg, 0.246 mmol) in ACN (1 mL) was added 3-fluoroazetidine hydrochloride (11 mg, 0.098 mmol) at room temperature. The resulting mixture was stirred at 50° C. for 2 h. The resulting mixture was concentrated under reduced pressure and purified by Prep-HPLC with the following conditions (Column: XBridge Prep OBD C18 Column 30*150 mm, 5 μm; Mobile Phase A: Water (10 mmol/L NH₄HCO₃+0.05% NH₃·H₂O), Mobile Phase B: MeOH; Flow rate: 60 mL/min; Gradient: 45% B to 62% B in 10 min; Wave Length: 254 nm; RT1(min): 9.88) to afford (7R,14R)-1-(difluoromethoxy)-11-(3-(3-fluoroazetidin-1-yl)prop-1-yn-1-yl)-6-(methyl-d3)-6,7-dihydro-7,14-methanobenzo[f]benzo[4,5]imidazo[1,2-a][1,4]diazocin-5(14H)-one (9 mg, 23%) as a white solid. MS ESI calculated for C₂₅H₁₈D₃F₃N₄O₂ [M+H]⁺, 470.18 found 470.15. ¹H NMR (400 MHz, Chloroform-d) δ 8.56-8.43 (m, 1H), 7.64 (d, J=8.4 Hz, 1H), 7.58 (s, 1H), 7.43 (d, J=8.2 Hz, 1H), 7.38-7.31 (m, 2H), 7.07-6.61 (m, 1H), 6.22 (d, J=7.2 Hz, 1H), 5.31-5.06 (m, 1H), 4.93 (d, J=7.1 Hz, 1H), 3.79-3.67 (m, 2H), 3.63-3.38 (m, 5H), 2.86 (d, J=13.5 Hz, 1H). ¹⁹F NMR (377 MHz, Chloroform-d) δ −79.96, −80.40, −81.31, −81.76, −179.22.

### Example 233: (7R,14R)-11-(4-(3,3-difluoroazetidin-1-yl)but-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

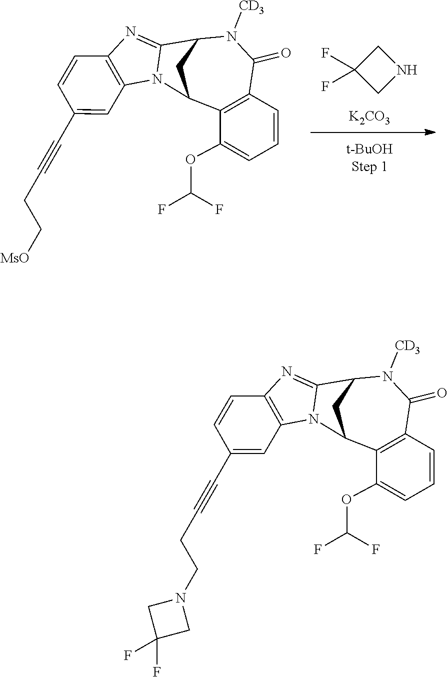

A solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)but-3-yn-1-yl methanesulfonate (40 mg, 0.08 mmol), 3,3-difluoroazetidine (15 mg, 0.16 mmol) and K₂CO₃ (33 mg, 0.24 mmol) in t-BuOH (1 mL) was stirred for 48 h at 80° C. The resulting mixture was concentrated under reduced pressure and purified by Prep-HPLC with the following conditions (Column: XBridge Prep Phenyl OBD Column 19*250 mm, 5 m; Mobile Phase A: Water (10 mmol/L NH₄HCO₃), Mobile Phase B: MEOH; Flow rate: 60 mL/min; Gradient: 50% B to 65% B in 10 min; Wave Length: 254 nm; RT1 (min): 13.27) to afford (7R,14R)-11-(4-(3,3-difluoroazetidin-1-yl)but-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (7 mg, 17%) as a white solid. MS ESI calculated for C2,H₁₉D₃F₄N₄O₂ [M+H]⁺, 502.19. found 502.20. ¹H NMR (400 MHz, Chloroform-d) δ 8.53-8.43 (m, 1H), 7.70-7.59 (m, 1H), 7.50 (s, 1H), 7.43 (d, J=8.2 Hz, 1H), 7.38-7.31 (m, 1H), 7.25-7.22 (m, 1H), 7.05-6.65 (m, 1H), 6.20 (d, J=7.2 Hz, 1H), 4.93 (d, J=7.1 Hz, 1H), 3.84 (t, J=11.8 Hz, 4H), 3.53-3.37 (m, 1H), 2.92 (t, J=6.8 Hz, 2H), 2.85 (d, J=13.6 Hz, 1H), 2.63 (t, J=6.7 Hz, 2H). ¹⁹F NMR (377 MHz, Chloroform-d) δ −80.09, −80.53, −81.04, −81.49, −98.56.

### Example 234: (7R,14R)-11-((1-cyclopropylpyrrolidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

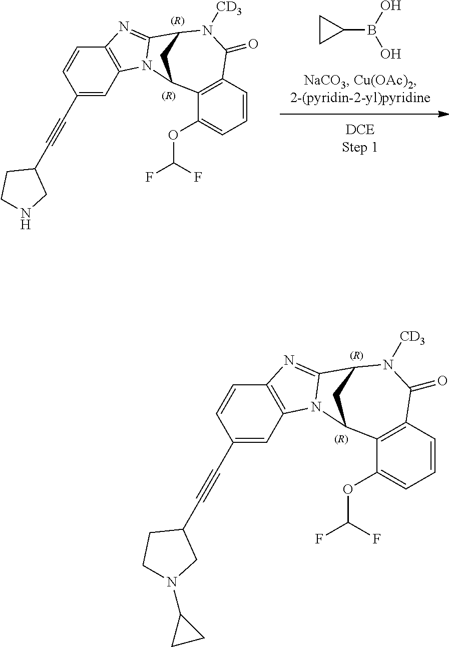

A solution of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (60 mg, 0.13 mmol), cyclopropylboronic acid (46 mg, 0.53 mmol), Na₂CO₃ (28 mg, 0.27 mmol), Cu(OAc)₂ (24 mg, 0.13 mmol) and 2-(pyridin-2-yl)pyridine (21 mg, 0.13 mmol) in DCE (1 mL) was stirred for 2 h at 70° C. under oxygen atmosphere. The resulting mixture was concentrated under reduced pressure. The residue was purified by Prep-TLC (CH₂Cl₂/MeOH, 10/1) followed by Prep-HPLC with the following conditions (Column: XBridge Prep Phenyl OBD Column 19*250 mm, 5 m; Mobile Phase A: Water (10 mmol/L NH₄HCO₃), Mobile Phase B: ACN; Flow rate: 60 mL/min; Gradient: 32% B to 47% B in 10 min; Wave Length: 254 nm; RT1 (min): 10.91) to afford (7R,14R)-11-((1-cyclopropylpyrrolidin-3-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (4 mg, 5%) as a white solid. MS ESI calculated for C₂H₂₃D₃F₂N₄O₂[M+H]⁺, 492,22. found 492.25. ¹H NMR (400 MHz, Chloroform-d) δ 8.51-8.45 (m, 1H), 7.60 (d, J=8.5 Hz, 1H), 7.48 (t, J=1.0 Hz, 1H), 7.41 (d, J=8.2 Hz, 1H), 7.37-7.30 (m, 1H), 7.29-7.24 (m, 1H), 7.05-6.64 (m, 1H), 6.19 (d, J=7.3 Hz, 1H), 4.92 (d, J=7.1 Hz, 1H), 3.50-3.40 (m, 1H), 3.33-3.15 (m, 2H), 3.03-2.94 (m, 1H), 2.88-2.70 (m, 3H), 2.38-2.24 (m, 1H), 2.10-1.92 (m, 1H), 1.88-1.78 (m, 1H), 0.65-0.40 (m, 4H). ¹⁹F NMR (377 MHz, Chloroform-d) δ −79.88, −80.32, −81.16, −81.61.

### Example 235: (7R,14R)-1-(difluoromethoxy)-11-(3-((S)-3-fluoropyrrolidin-1-yl)prop-1-yn-1-yl)-6-(methyl-d₃)-6,7-dihydro-7,14-methanobenzo[f]benzo[4,5]imidazo[1,2-a][1,4]diazocin-5(14H)-one

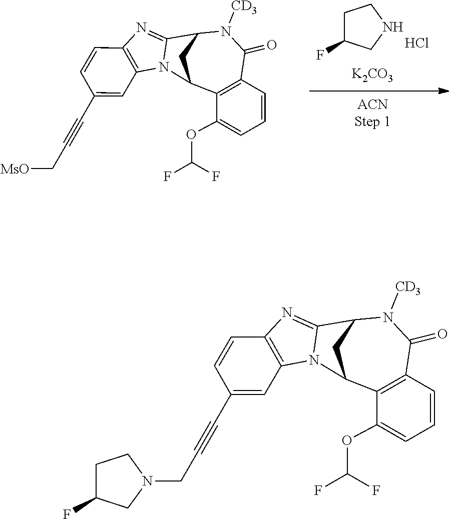

To a stirred solution of 3-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)prop-2-yn-1-yl methanesulfonate (40 mg, 0.082 mmol) and K₂CO₃ (34 mg, 0.246 mmol) in ACN (1 mL) was added (3S)-3-fluoropyrrolidine hydrochloride (12 mg, 0.098 mmol). The resulting mixture was stirred at 50° C. for 2 h. The mixture was filtered, the filter cake was washed with EtOAc (2 mL). The filtrate was concentrated under reduced pressure. The residue was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: MeOH; Flow rate: 60 mL/min; Gradient: 49% B to 66% B in 10 min; Wave Length: 254 nm; RT1(min): 11.08) to afford (7R,14R)-1-(difluoromethoxy)-11-(3-((S)-3-fluoropyrrolidin-1-yl)prop-1-yn-1-yl)-6-(methyl-d₃)-6,7-dihydro-7,14-methanobenzo[f]benzo[4,5]imidazo[1,2-a][1,4]diazocin-5(14H)-one as a white solid (9 mg, 22%). MS ESI calculated for C₂₆H₂₀D₃F₃N₄O₂[M+H]⁺, 484.20 found 484.15. ¹H NMR (400 MHz, Chloroform-d) δ 8.48 (d, J=8.2 Hz, 1H), 7.62 (d, J=8.4 Hz, 1H), 7.55 (d, J=1.5 Hz, 1H), 7.42 (t, J=8.2 Hz, 1H), 7.36-7.25 (m, 2H), 6.85 (t, J=72.8 Hz, 1H), 6.21 (d, J=7.2 Hz, 1H), 5.37-5.13 (m, 1H), 4.93 (d, J=7.1 Hz, 1H), 3.78 (s, 2H), 3.49-3.37 (m, 1H), 3.19-3.01 (m, 3H), 2.86 (d, J=13.5 Hz, 2H), 2.24-2.08 (m, 2H). ¹⁹F NMR (377 MHz, Chloroform-d) δ −80.04, −80.49, −81.11, −81.56.

### Example 236: (7R,14R)-1-(difluoromethoxy)-11-(3-((R)-3-fluoropyrrolidin-1-yl)prop-1-yn-1-yl)-6-(methyl-d₃)-6,7-dihydro-7,14-methanobenzo[f]benzo[4,5]imidazo[1,2-a][1,4]diazocin-5(14H)-one

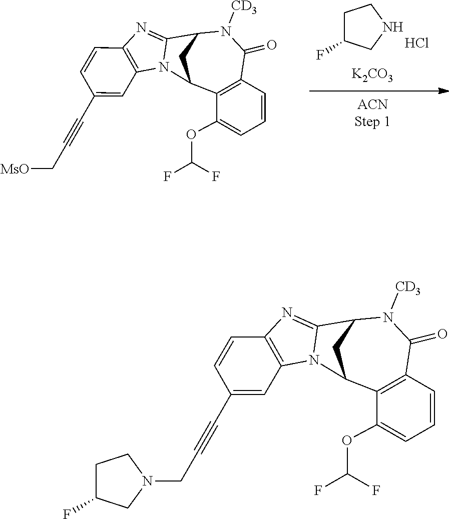

To a stirred solution of 3-((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)prop-2-yn-1-yl methanesulfonate (40 mg, 0.082 mmol) and K₂CO₃ (34 mg, 0.246 mmol) in ACN (2 mL) was added (R)-3-fluoropyrrolidine hydrochloride (18 mg, 0.098 mmol) at room temperature. The resulting mixture was stirred for 2 h at 50° C. The mixture was filtered, the filter cake was washed with EtOAc (2 mL). The filtrate was concentrated under reduced pressure. The residue was purified by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: MeOH; Flow rate: 60 mL/min; Gradient: 49% B to 66% B in 10 min; Wave Length: 254 nm; RT1(min):11.08) to (7R,14R)-1-(difluoromethoxy)-11-(3-((R)-3-fluoropyrrolidin-1-yl)prop-1-yn-1-yl)-6-(methyl-d₃)-6,7-dihydro-7,14-methanobenzo[f]benzo[4,5]imidazo[1,2-a][1,4]diazocin-5(14H)-one as a white solid (7 mg, 18%). MS ESI calculated for C₂₆H₂₀D₃F₃N₄O₂[M+H]⁺, 484.20 found 484.15. ¹H NMR (400 MHz, Chloroform-d) δ 8.48 (d, J=8.0 Hz, 1H), 7.63 (d, J=8.4 Hz, 1H), 7.55 (s, 1H), 7.45-7.39 (m, 1H), 7.35-7.28 (m, 2H), 6.85 (t, J=72.7 Hz, 1H), 6.21 (d, J=7.2 Hz, 1H), 5.36-5.14 (m, 1H), 4.93 (d, J=7.1 Hz, 1H), 3.80 (s, 2H), 3.48-3.39 (m, 1H), 3.23-3.03 (m, 3H), 2.86 (d, J=13.6 Hz, 2H), 2.34-2.10 (m, 2H). ¹⁹F NMR (377 MHz, Chloroform-d) δ −80.06, −80.51, −81.10, −81.55.

### Example 237: (7R,14R)-1-(difluoromethoxy)-6-(methyl-d3)-11-((1-(oxetan-3-yl)pyrrolidin-3-yl)ethynyl)-6,7-dihydro-7,14-methanobenzo[f]benzo[4,5]imidazo[1,2-a][1,4]diazocin-5(14H)-one

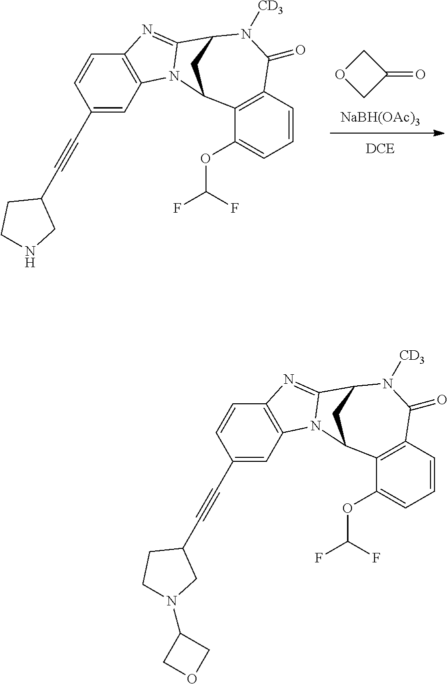

To a stirred solution of (7R,14R)-1-(difluoromethoxy)-6-(methyl-d3)-11-(pyrrolidin-3-ylethynyl)-6,7-dihydro-7,14-methanobenzo[f]benzo[4,5]imidazo[1,2-a][1,4]diazocin-5(14H)-one (40 mg, 0.09 mmol) and 3-oxetanone (8 mg, 0.11 mmol) in DCE (1 mL) was added NaBH(OAc)3 (30 mg, 0.14 mmol). The resulting mixture was stirred for 16 h at room temperature. The reaction was quenched by the addition of water (10 mL). The mixture was extracted with EtOAc (2×10 mL). The combined organic layers were washed with brine (10 mL), dried over anhydrous Na₂SO₄. After filtration, the filtrate was concentrated under reduced pressure. The residue was purified by Prep-TLC (EA) followed by Prep-HPLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min; Gradient: 23% B to 40% B in 10 min; Wave Length: 254 nm; RT1(min): 11.3) to afford (7R,14R)-1-(difluoromethoxy)-6-(methyl-d3)-11-((1-(oxetan-3-yl)pyrrolidin-3-yl)ethynyl)-6,7-dihydro-7,14-methanobenzo[f]benzo[4,5]imidazo[1,2-a][1,4]diazocin-5(14H)-one (5 mg, 11%) as a white solid. MS ESI calculated for C₂₈H₂₃D₃F₂N₄O₃ [M+H]⁺, 508.22. found 508.10. ¹H NMR (400 MHz, Chloroform-d) δ 8.51-8.44 (m, 1H), 7.65-7.58 (m, 1H), 7.48 (d, J=1.5 Hz, 1H), 7.42 (t, J=8.2 Hz, 1H), 7.33 (d, J=8.1 Hz, 1H), 7.24 (d, J=1.5 Hz, 1H), 6.84 (t, J=72.3 Hz, 1H), 6.19 (d, J=7.2 Hz, 1H), 4.92 (d, J=7.1 Hz, 1H), 4.78-4.62 (m, 4H), 3.80-3.72 (m, 1H), 3.50-3.38 (m, 1H), 3.30-3.19 (m, 1H), 3.08 (t, J=8.3 Hz, 1H), 2.85 (d, J=13.6 Hz, 1H), 2.81-2.73 (m, 1H), 2.61-2.46 (m, 2H), 2.35-2.22 (m, 1H), 2.09-1.96 (m, 1H). ¹⁹F NMR (377 MHz, Chloroform-d) δ −79.89, −80.33, −81.09, −81.54.

### Example 238: (7R,14R)-1-(difluoromethoxy)-11-((1-(1,1-difluoropropan-2-yl)azetidin-3-yl)ethynyl)-6-(methyl-d3)-6,7-dihydro-7,14-methanobenzo[f]benzo[4,5]imidazo[1,2-a][1,4]diazocin-5(14H)-one

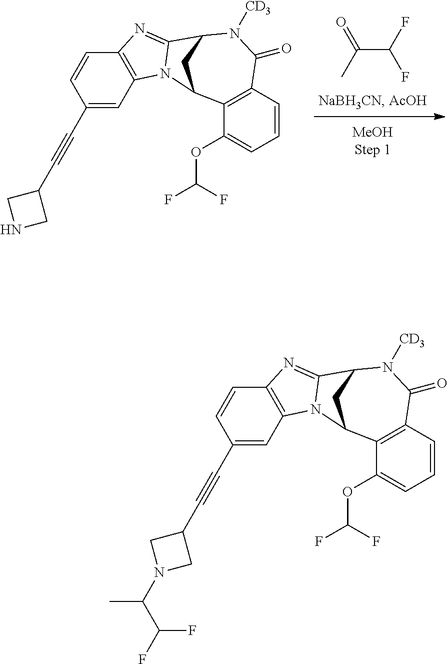

To a solution of (7R,14R)-11-(azetidin-3-ylethynyl)-1-(difluoromethoxy)-6-(methyl-d3)-6,7-dihydro-7,14-methanobenzo[f]benzo[4,5]imidazo[1,2-a][1,4]diazocin-5(14H)-one (40 mg, 0.091 mmol), 1,1-difluoropropan-2-one (13 mg, 0.137 mmol) and AcOH (16 mg, 0.273 mmol) in MeOH (0.5 mL) was added NaBH₃CN (9 mg, 0.137 mmol). The resulting mixture was stirred for 1 h at room temperature. The mixture was concentrated under reduced pressure and purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: CH₃CN; Flow rate: 60 mL/min; Gradient: 31% B to 51% B in 10 min; Wave Length: 254/220 nm; RT1(min): 10.62) to afford (7R,14R)-1-(difluoromethoxy)-11-((1-(1,1-difluoropropan-2-yl)azetidin-3-yl)ethynyl)-6-(methyl-d3)-6,7-dihydro-7,14-methanobenzo[f]benzo[4,5]imidazo[1,2-a][1,4]diazocin-5(14H)-one (26 mg, 55%) as a white solid. MS ESI calculated for C₂₇H₂₁D₃F₄N₄O₂ [M+H]⁺, 516.20. found 516.10. ¹H NMR (400 MHz, Chloroform-d) δ 8.48 (d, J=8.2, 1.3 Hz, 1H), 7.61 (d, J=8.4 Hz, 1H), 7.49 (s, 1H), 7.42 (t, J=8.2 Hz, 1H), 7.37-7.27 (m, 2H), 6.84 (t, J=73.2 Hz, 1H), 6.20 (d, J=7.2 Hz, 1H), 5.66-5.33 (m, 1H), 4.93 (d, J=7.1 Hz, 1H), 3.78 (t, J=7.3 Hz, 1H), 3.71-3.64 (m, 1H), 3.53-3.38 (m, 2H), 3.34-3.20 (m, 2H), 2.85 (d, J=13.6 Hz, 1H), 2.70-2.58 (m, 1H), 1.03 (d, J=6.6 Hz, 3H). ¹⁹F NMR (377 MHz, Chloroform-d) δ −79.87, −80.32, −81.04, −81.48, −122.72, −123.48, −124.21, −124.98.

### Example 239 and 240: (7R,14R)-1-(difluoromethoxy)-11-((S or R)-5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one and (7R,14R)-1-(difluoromethoxy)-11-((R or S)-5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one

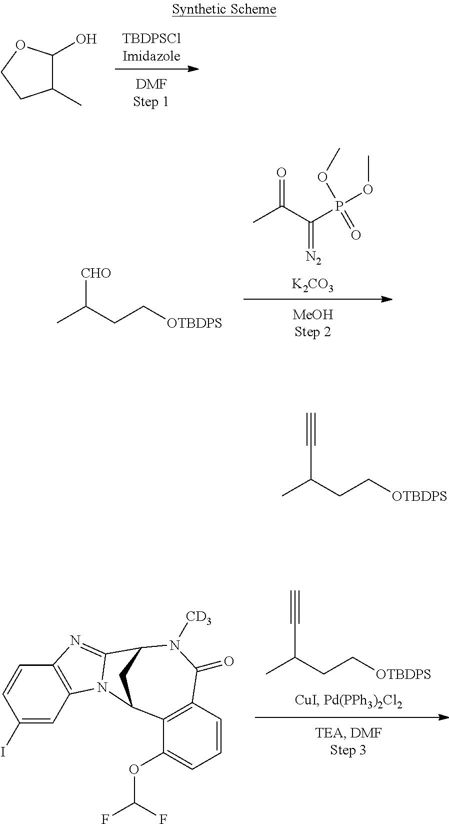

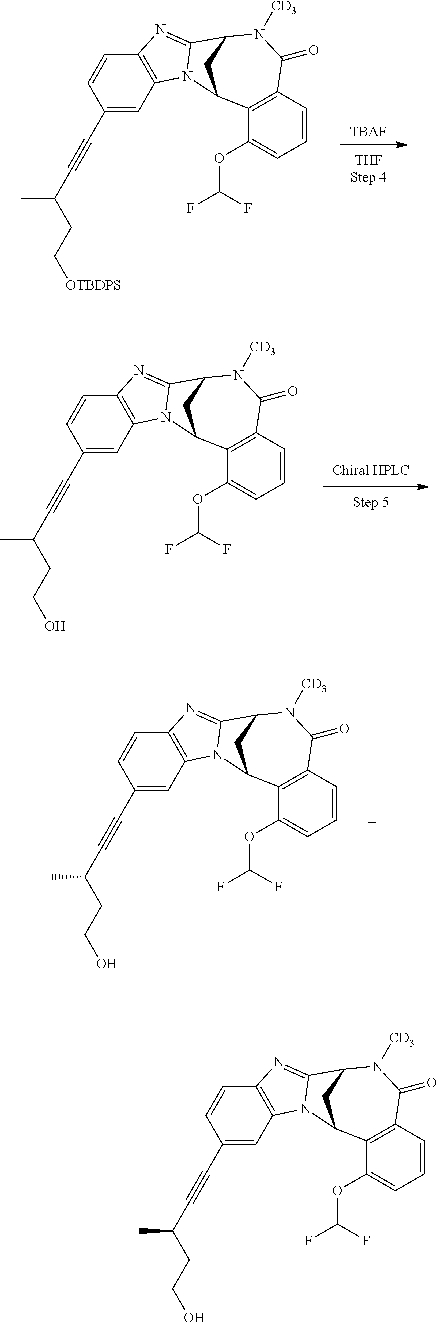

### Preparation 239A: 4-((tert-butyldiphenylsilyl)oxy)-2-methylbutanal

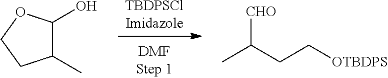

To a stirred mixture of 3-methyltetrahydrofuran-2-ol (300 mg, 2.937 mmol) and 1H-imidazole (200 mg, 2.937 mmol) in DMF (10 mL) was added TBDPSCl (807 mg, 2.937 mmol). The resulting mixture was stirred for 1 h at 60° C. Then to the mixture were added 1H-imidazole (100 mg, 1.46 mmol) and TBDPSCl (404 mg, 1.46 mmol). The reaction was stirred for overnight at 60° C. The mixture was diluted with water (5 mL) and extracted with EtOAc (3×10 mL). The combined organic layers were washed with brine (10 mL), dried over anhydrous Na₂SO₄. After filtration, the filtrate was concentrated under reduced pressure. The residue was purified by Prep-TLC (PE/EA 10:1) to afford 4-((tert-butyldiphenylsilyl)oxy)-2-methylbutanal (308 mg, 31%) as a colorless oil. MS ESI calculated for C₂₁H₂₈O₂Si [M+H]⁺, 341.49. found N/A. ¹H NMR (400 MHz, Chloroform-d) δ 9.67 (d, J=1.6 Hz, 1H), 7.73-7.61 (m, 4H), 7.51-7.30 (m, 6H), 3.81-3.62 (m, 2H), 2.64-2.48 (m, 1H), 2.08-1.94 (m, 1H), 1.69-1.57 (m, 1H), 1.08 (d, J=7.1 Hz, 3H), 1.04 (s, 9H).

### Preparation 239B: tert-butyl((3-methylpent-4-yn-1-yl)oxy)diphenylsilane

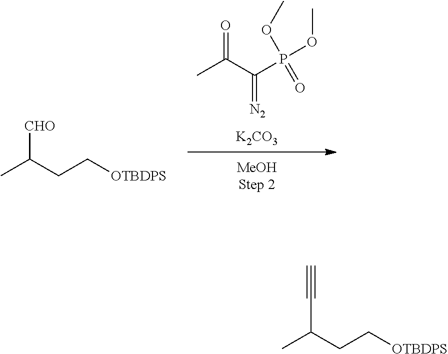

To a stirred mixture of 4-[(tert-butyldiphenylsilyl)oxy]-2-methylbutanal (300 mg, 0.881 mmol) and K₂CO₃ (244 mg, 1.762 mmol) in MeOH (5 mL) was added dimethyl (1-diazo-2-oxopropyl)phosphonate (254 mg, 1.321 mmol) at room temperature. The resulting mixture was stirred for 1 h at room temperature. The mixture was diluted with water (10 mL) and extracted with EtOAc (3×10 mL). The combined organic layers were washed with brine (1×15 mL), dried over anhydrous Na₂SO₄. After filtration, the filtrate was concentrated under reduced pressure. The residue was purified by Prep-TLC (PE/EA 10:1) to afford tert-butyl[(3-methylpent-4-yn-1-yl)oxy]diphenylsilane (294 mg, 98%) as a colorless oil. MS ESI calculated for C₂₂H₂₈OSi [M+H]⁺, 337.19. found N/A. ¹H NMR (400 MHz, Chloroform-d) δ 7.70-7.65 (m, 4H), 7.42-7.37 (m, 6H), 3.87-3.67 (m, 2H), 2.79-2.69 (m, 1H), 1.99 (d, J=2.4 Hz, 1H), 1.74-1.66 (m, 2H), 1.19 (d, J=7.0 Hz, 3H), 1.05 (s, 9H).

### Preparation 239C: (7R,14R)-11-(5-((tert-butyldiphenylsilyl)oxy)-3-methylpent-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)one

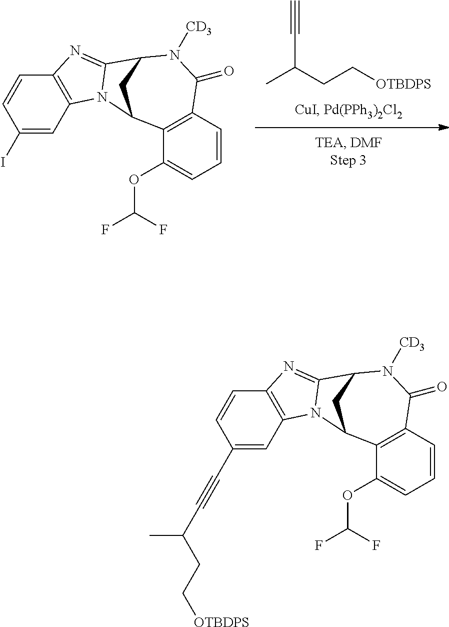

To a stirred mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (200 mg, 0.413 mmol) and tert-butyl[(3-methylpent-4-yn-1-yl)oxy]diphenylsilane (208 mg, 0.619 mmol) in DMF (2.5 mL) and Et₃N (2.5 mL) were added CuI (8 mg, 0.041 mmol) and Pd(PPh₃)₂Cl₂ (29 mg, 0.041 mmol) at room temperature. The resulting mixture was stirred for 1 h at 80° C. under nitrogen atmosphere. The mixture was diluted with water (15 mL) and extracted with EtOAc (3×15 mL). The combined organic layers were washed with brine (1×15 mL), dried over anhydrous Na₂SO₄. After filtration, the filtrate was concentrated under reduced pressure. The residue was purified by Prep-TLC (CH₂Cl₂/MeOH 10:1) to afford (7R,14R)-11-(5-((tert-butyldiphenylsilyl)oxy)-3-methylpent-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (199 mg, 70%) as a yellow solid. MS ESI calculated for C₄₁H₃₈D₃F₂N₃O₃Si [M+H]⁺, 693.31. found 693.35. ¹H NMR (400 MHz, Chloroform-d) δ 8.49 (d, J=8.1 Hz, 1H), 7.72-7.27 (m, 15H), 6.89-6.41 (m, 1H), 6.23 (d, J=7.2 Hz, 1H), 5.09 (d, J=7.1 Hz, 1H), 3.98-3.78 (m, 2H), 3.53-3.43 (m, 1H), 3.05-2.96 (m, 1H), 2.91-2.86 (m, 1H), 1.87-1.70 (m, 2H), 1.28 (d, J=6.9 Hz, 3H), 1.05 (s, 9H).

### Preparation 239D: (7R,14R)-1-(difluoromethoxy)-11-(5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one

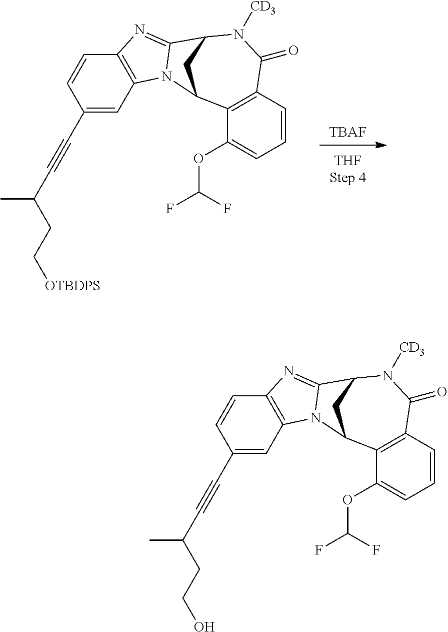

To a stirred solution of (7R,14R)-11-(5-((tert-butyldiphenylsilyl)oxy)-3-methylpent-1-yn-1-yl)-1-(difluoromethoxy)-6-(methyl-d₃)-6,7-dihydro-7,14-methanobenzo[f]benzo[4,5]imidazo[1,2-a][1,4]diazocin-5(14H)-one (190 mg, 0.274 mmol) in THF (4 mL) was added TBAF (0.3 mL, 0.329 mmol, 1 M in THF). The resulting mixture was stirred for 2 h at room temperature. The mixture was diluted with water (20 mL) and extracted with EtOAc (3×25 mL). The combined organic layers were washed with brine (3×20 mL), dried over anhydrous Na₂SO₄. After filtration, the filtrate was concentrated under reduced pressure. The residue was purified by Prep-TLC (CH₂Cl₂/MeOH 10:1) to afford (7R,14R)-1-(difluoromethoxy)-11-(5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one (120 mg, 96%) as a yellow solid. MS ESI calculated for C₂₅H₂₀D₃F₂N₃O₃[M+H]⁺, 455.19 found 455.15.

### Example 239 and 240: (7R,14R)-1-(difluoromethoxy)-11-((S or R)-5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one and (7R,14R)-1-(difluoromethoxy)-11-((R or S)-5-hydroxy-3-methylpent-1-yn-1-yl)-6-(methyl-d₃)-6,7-dihydro-7,14-methanobenzo[f]benzo[4,5]imidazo[1,2-a][1,4]diazocin-5(14H)-one

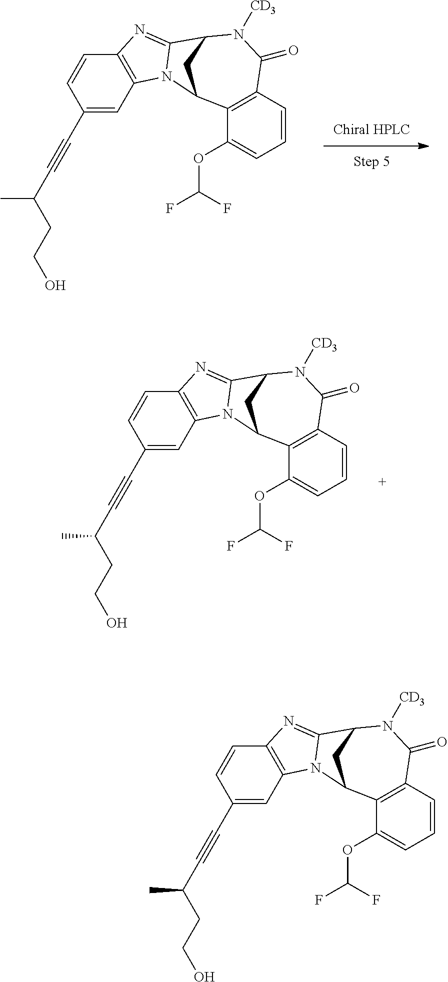 [SMILES: [2H]C([2H])([2H])N1C(=O)c2cccc(OC(F)F)c2[C@H]2C[C@@H]1c1nc3ccc(C#CC(C)CCO)cc3n12 ; [2H]C([2H])([2H])N1C(=O)c2cccc(OC(F)F)c2[C@H]2C[C@@H]1c1nc3ccc(C#C[C@@H](C)CCO)cc3n12 ; [2H]C([2H])([2H])N1C(=O)c2cccc(OC(F)F)c2[C@H]2C[C@@H]1c1nc3ccc(C#C[C@H](C)CCO)cc3n12]

The mixture (120 mg) was purified by SFC with the following conditions (Column: NB_CHIRALPAK AD-H, 3*25 cm, 5 μm; Mobile Phase A: CO₂, Mobile Phase B: ETOH (0.1% 2M NH₃-MeOH); Flow rate: 100 mL/min; Gradient: isocratic 20% B; Wave Length: 220 nm; RT1(min): 10.05; RT2(min): 11.62). The first peak afforded (33 mg, 27%) as a white solid. MS ESI calculated for C₂₅H₂₀D₃F₂N₃O₃[M+H]⁺, 455.19 found 455.10. ¹H NMR (400 MHz, Chloroform-d) δ 8.50-8.45 (m, 1H), 7.63-7.59 (m, 1H), 7.51-7.48 (m, 1H), 7.42 (t, J=8.2 Hz, 1H), 7.36-7.31 (m, 1H), 7.30-7.27 (m, 1H), 7.05-6.68 (m, 1H), 6.22 (d, J=7.2 Hz, 1H), 4.99 (d, J=7.1 Hz, 1H), 3.94-3.83 (m, 2H), 3.50-3.39 (m, 1H), 2.92-2.82 (m, 2H), 1.87-1.72 (m, 2H), 1.31 (d, J=6.9 Hz, 3H). ¹⁹F NMR (377 MHz, Chloroform-d) δ −80.02, −80.47, −81.33, −81.77.

The second peak afforded (16 mg, 14%) as a white solid. MS ESI calculated for C₂₅H₂₀D₃F₂N₃O₃[M+H]⁺, 455.19 found 455.05. ¹H NMR (400 MHz, Chloroform-d) δ 8.48-8.43 (m, 1H), 7.59 (d, J=8.5 Hz, 1H), 7.49-7.45 (m, 1H), 7.38 (t, J=8.2 Hz, 1H), 7.33-7.28 (m, 1H), 7.25-7.19 (m, 1H), 7.05-6.66 (m, 1H), 6.17 (d, J=7.1 Hz, 1H), 4.92 (d, J=7.1 Hz, 1H), 3.95-3.82 (m, 2H), 3.46-3.35 (m, 1H), 2.93-2.78 (m, 2H), 1.88-1.70 (m, 2H), 1.31 (d, J=6.9 Hz, 3H). ¹⁹F NMR (377 MHz, Chloroform-d) δ −80.00, −80.45, −81.36, −81.80.

### Example 241: (7R,14R)-11-(3-(cyclopropylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

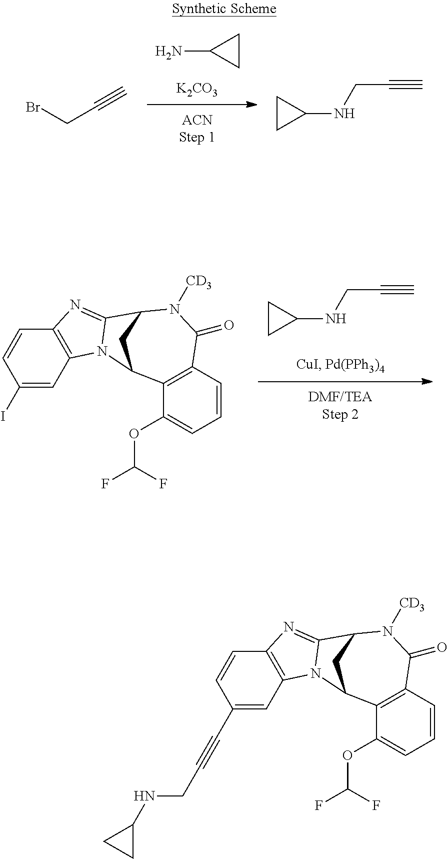

### Preparation 241A: N-(prop-2-yn-1-yl)cyclopropanamine

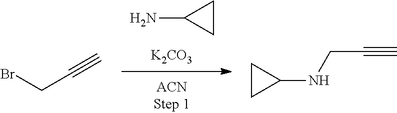

To a stirred solution of cyclopropanamine (0.48 g, 8.406 mmol) and K₂CO₃ (2.32 g, 16.812 mmol) in ACN (20 mL) was added 3-bromoprop-1-yne (1.00 g, 8.406 mmol). The resulting mixture was stirred for overnight at 50° C. The mixture was allowed to cool down to room temperature, and diluted with water (30 mL). The mixture was acidified to pH 4-5 with 2 M HCl (aq.), and washed with EtOAc (3×30 mL). The aqueous layer was basified to pH 8 with saturated NaHCO₃(aq.), and extracted with CH₂Cl₂ (4×30 mL). The combined organic layers were washed with brine (1×50 mL), dried over anhydrous Na₂SO₄. After filtration, the filtrate was concentrated under reduced pressure. This resulted in N-(prop-2-yn-1-yl)cyclopropanamine (200 mg, 25%) as a light yellow liquid. MS ESI calculated for C₆H₉N [M+H]⁺, 96.07. found 95.95. ¹H NMR (400 MHz, Chloroform-d) δ 3.58 (d, J=2.4 Hz, 1H), 3.46 (d, J=2.5 Hz, 2H), 2.42-2.33 (m, 1H), 2.25-2.21 (m, 1H), 0.49-0.44 (m, 2H), 0.42-0.37 (m, 2H).

### Example 241: (7R,14R)-11-(3-(cyclopropylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

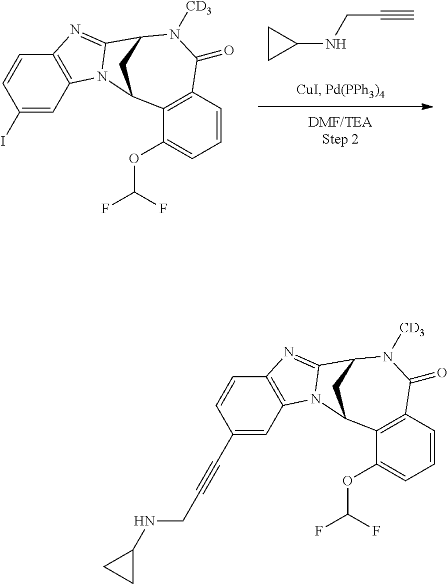

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (60 mg, 0.124 mmol) and N-(prop-2-yn-1-yl)cyclopropanamine (35 mg, 0.372 mmol) in DMF (0.5 mL) and TEA (0.5 mL) were added Pd(PPh₃)₄(14 mg, 0.012 mmol) and CuI (5 mg, 0.025 mmol) at room temperature. The resulting mixture was stirred for 2 h at 80° C. under nitrogen atmosphere. The mixture was allowed to cool down to room temperature, diluted with water (10 mL), and extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 50% gradient in 20 min; detector, 254 nm to afford (7R,14R)-11-(3-(cyclopropylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (8 mg, 14%) as a white solid. MS ESI calculated for C₂₅H₁₉D₃F₂N₄O₂ [M+H]⁺, 452.19. found 452.20. ¹H NMR (400 MHz, Chloroform-d) δ 8.51-8.44 (m, 1H), 7.65-7.58 (m, 1H), 7.55-7.51 (m, 1H), 7.44-7.38 (m, 1H), 7.35-7.28 (m, 2H), 6.83 (t, J=73.2 Hz, 1H), 6.19 (d, J=7.2 Hz, 1H), 4.92 (d, J=7.0 Hz, 1H), 3.69 (s, 2H), 3.49-3.36 (m, 1H), 2.84 (d, J=13.6 Hz, 1H), 2.48-2.40 (m, 1H), 1.99-1.91 (m, 1H), 0.54-0.47 (m, 2H), 0.47-0.41 (m, 2H). ¹⁹F NMR (377 MHz, Chloroform-d) δ −79.93, −80.37, −81.05, −81.50.

### Example 242: (7R,14R)-1-(difluoromethoxy)-6-(methyl-d3)-11-(3-(oxetan-3-ylamino)prop-1-yn-1-yl)-6,7-dihydro-7,14-methanobenzo[f]benzo[4,5]imidazo[1,2-a][1,4]diazocin-5(14H)-one

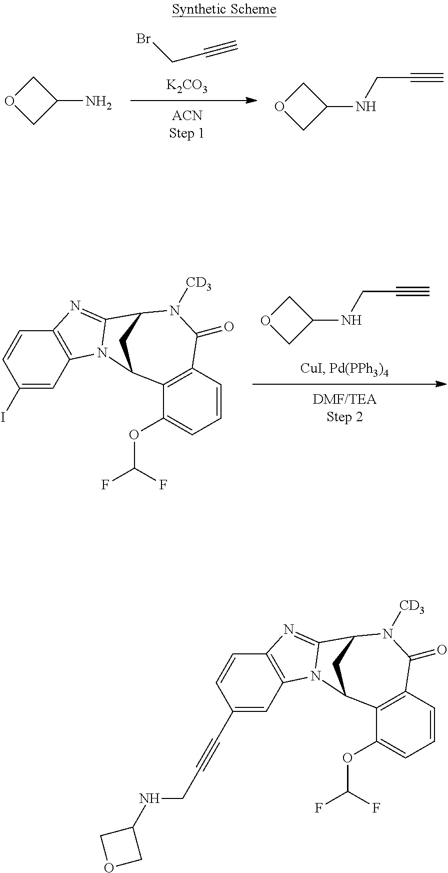

### Preparation 242A: N-(prop-2-yn-1-yl)oxetan-3-amine

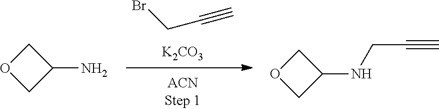

To a stirred solution of oxetan-3-amine (614 mg, 8.406 mmol) and K₂CO₃ (2.32 g, 16.812 mmol) in ACN (20 mL) was added 3-bromoprop-1-yne (1.00 g, 8.406 mmol). The resulting mixture was stirred for overnight at 50° C. The mixture was diluted with water (30 mL), and acidified to pH 4-5 with 2 M HCl (aq.). The resulting mixture was washed with EtOAc (3×30 mL). The aqueous layer was basified to pH 8 with saturated NaHCO₃(aq.), and extracted with CH₂Cl₂ (4×30 mL). The combined organic layers were washed with brine (1×50 mL), dried over anhydrous Na₂SO₄. After filtration, the filtrate was concentrated under reduced pressure to afford N-(prop-2-yn-1-yl)oxetan-3-amine (200 mg, 21%) as a light yellow liquid. MS ESI calculated for C₆H₉NO [M+H]⁺, 112.07. found 112.15. ¹H NMR (400 MHz, Chloroform-d) δ 4.89-4.82 (m, 2H), 4.57-4.50 (m, 2H), 4.14-4.08 (m, 1H), 3.41 (d, J=2.4 Hz, 2H), 2.22-2.19 (m, 1H).

### Example 242: (7R,14R)-1-(difluoromethoxy)-6-(methyl-d3)-11-(3-(oxetan-3-ylamino)prop-1-yn-1-yl)-6,7-dihydro-7,14-methanobenzo[f]benzo[4,5]imidazo[1,2-a][1,4]diazocin-5(14H)-one

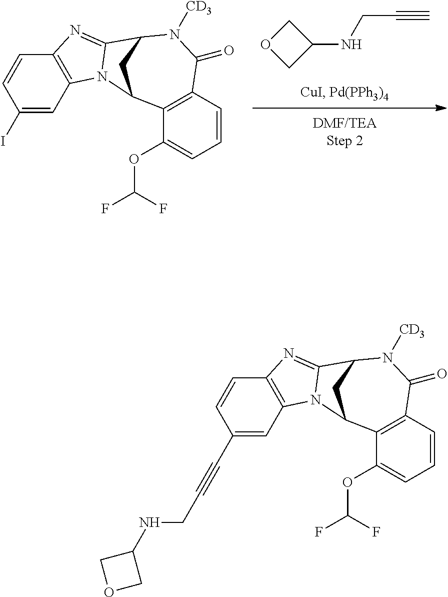

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (60 mg, 0.124 mmol) and N-(prop-2-yn-1-yl)oxetan-3-amine (41 mg, 0.372 mmol) in DMF (0.5 mL) and TEA (0.5 mL) were added CuI (5 mg, 0.025 mmol) and Pd(PPh₃)₄(14 mg, 0.012 mmol). The resulting mixture was concentrated under reduced pressure. The residue was purified by silica gel column chromatography, eluted with CH₂Cl₂/MeOH (0% to 10%) followed by reversed-phase flash chromatography with the following conditions: column, C18 silica gel; mobile phase, CH₃CN in Water (10 mmol/L NH₄HCO₃), 30% to 50% gradient in 20 min; detector, 254 nm to afford (7R,14R)-1-(difluoromethoxy)-6-(methyl-d3)-11-(3-(oxetan-3-ylamino)prop-1-yn-1-yl)-6,7-dihydro-7,14-methanobenzo[f]benzo[4,5]imidazo[1,2-a][1,4]diazocin-5(14H)-one (42 mg, 73%) as a white solid. MS ESI calculated for C₂₅H₁₉D₃F₂N₄O₃ [M+H]⁺, 468.18 found 468.20. ¹H NMR (400 MHz, Chloroform-d) δ 8.51-8.44 (m, 1H), 7.62 (d, J=8.4 Hz, 1H), 7.50 (s, 1H), 7.45-7.39 (m, 1H), 7.35-7.31 (m, 1H), 7.26-7.21 (m, 1H), 6.85 (t, J=72.8 Hz, 1H), 6.21 (d, J=7.2 Hz, 1H), 4.98-4.85 (m, 3H), 4.68-4.59 (m, 2H), 4.23-4.14 (m, 1H), 3.69 (s, 2H), 3.48-3.38 (m, 1H), 2.86 (d, J=13.6 Hz, 1H). ¹⁹F NMR (377 MHz, Chloroform-d)) δ −80.07, −80.52, −80.98, −81.43.

### Example 243, 244, 245 and 246: (7R,14R)-1-(difluoromethoxy)-11-((4R or 4S,5S or 5R)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((4S or 4S,5R or 5S)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one; (7R,14R)-1-(difluoromethoxy)-11-((4S or 4R,5S or 5R)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((4R or 4S,5R or 5S)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one

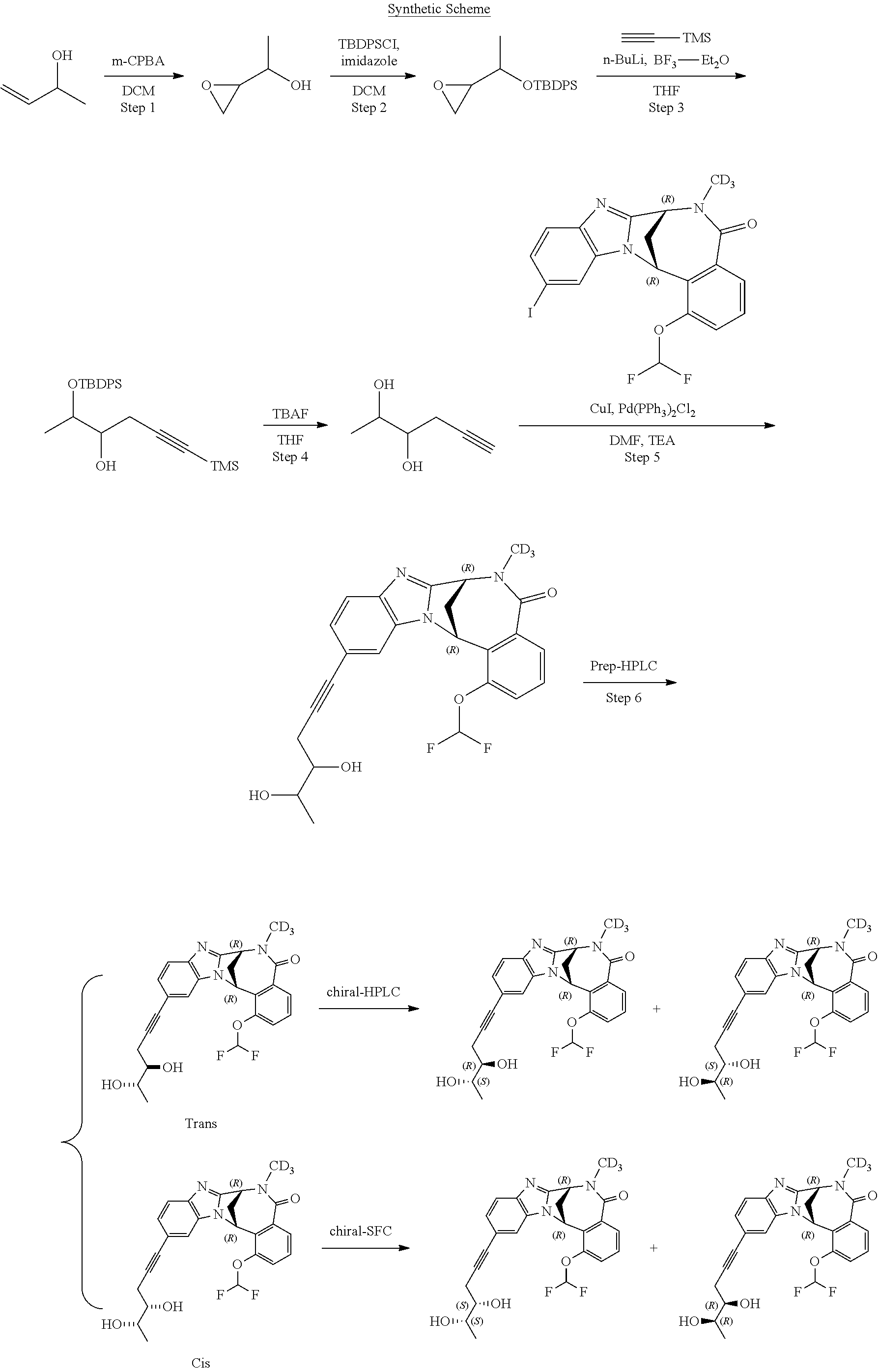

### Preparation 243A: 1-(oxiran-2-yl)ethan-1-ol

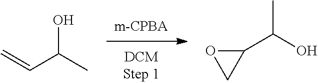

To a stirred solution of 3-buten-2-ol (5.00 g, 69.341 mmol) in DCM (170 mL) were added m-CPBA (17.60 g, 86.676 mmol) in portions at 0° C. The resulting mixture was stirred for 18 h at room temperature. The mixture was filtered, the filter cake was washed with DCM (2×30 mL). The filtrate was concentrated under reduced pressure. The residue was purified by silica gel column chromatography, eluted with PE/Et₂O (1:1) to afford 1-(oxiran-2-yl)ethanol (2.30 g, 38%) as a colorless oil. MS ESI calculated for C₄H₈O₂ [M+H]⁺, 89.05. found N/A. ¹H NMR (300 MHz, Chloroform-d) δ 4.07-3.54 (m, 1H), 3.07-2.93 (m, 1H), 2.87-2.77 (m, 1H), 2.80-2.66 (m, 1H), 1.35-1.21 (m, 3H).

### Preparation 243B: tert-butyl(1-(oxiran-2-yl)ethoxy)diphenylsilane

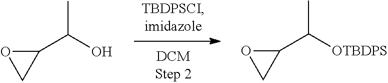

To a stirred mixture of 1-(oxiran-2-yl)ethanol (2.30 g, 26.105 mmol) and 1H-imidazole (2.13 g, 31.326 mmol) in DCM (40 mL) was added tert-butyl(chloro)diphenylsilane (8.61 g, 31.326 mmol) in portions at 0° C. The resulting mixture was stirred for 4 h at room temperature. The reaction was quenched with water (20 mL). The mixture was extracted with DCM (3×20 mL). The combined organic layers were dried over anhydrous Na₂SO₄. After filtration, the filtrate was concentrated under reduced pressure. The residue was purified by silica gel column chromatography, eluted with PE/EA (20:1) to afford tert-butyl[1-(oxiran-2-yl)ethoxy]diphenylsilane (6.70 g, 79%) as a colorless oil. MS ESI calculated for C₂₀H₂₆O₂Si [M+H]⁺, 327.17. found N/A. ¹H NMR (400 MHz, Chloroform-d) δ 7.76-7.63 (m, 4H), 7.47-7.32 (m, 6H), 3.69-3.54 (m, 1H), 3.08-2.81 (m, 1H), 2.77-2.51 (m, 1H), 2.54-2.22 (m, 1H), 1.23-1.09 (m, 3H), 1.08-1.03 (m, 9H).

### Preparation 243C: 2-((tert-butyldiphenylsilyl)oxy)-6-(trimethylsilyl)hex-5-yn-3-ol

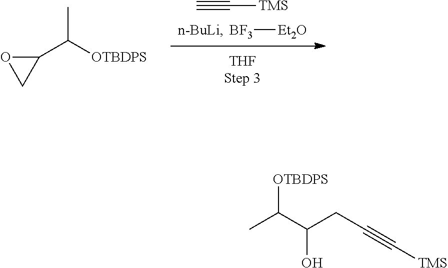

To a stirred solution of trimethylsilylacetylene (2.93 g, 29.861 mmol) in THF (100 mL) was added n-butyllithium (11.9 mL, 29.861 mmol, 2.5 mol/L in THF) dropwise at −78° C. under nitrogen atmosphere. The resulting mixture was stirred for 15 min at −78° C. under nitrogen atmosphere. To the above mixture was added a solution of tert-butyl[1-(oxiran-2-yl)ethoxy]diphenylsilane (6.50 g, 19.907 mmol) in THF (50 mL) and boron trifluoride diethyl etherate (3.69 mL, 29.861 mmol) dropwise at −78° C. The resulting mixture was stirred for additional 3 h at −78° C. under nitrogen atmosphere. The reaction was quenched with sat. NH₄C1 (aq.) at −78° C. The mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (20:1) to afford 2-[(tert-butyldiphenylsilyl)oxy]-6-(trimethylsilyl)hex-5-yn-3-ol (7.40 g, 88%) as a colorless oil. MS ESI calculated for C₂₅H₃₆O₂Si₂ [M+H]⁺, 425.23. found N/A. ¹H NMR (300 MHz, Chloroform-d) δ 7.76-7.64 (m, 4H), 7.50-7.34 (m, 6H), 4.08-3.95 (m, 1H), 3.81-3.54 (m, 1H), 2.59-2.42 (m, 2H), 1.08 (s, 9H), 1.04 (dd, J=6.3, 3.2 Hz, 3H), 0.13 (d, J=7.5 Hz, 9H).

### Preparation 243D: hex-5-yne-2,3-diol

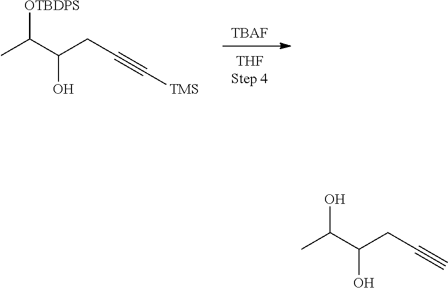

To a stirred solution of 2-[(tert-butyldiphenylsilyl)oxy]-6-(trimethylsilyl)hex-5-yn-3-ol (6.50 g, 15.304 mmol) in THF (120 mL) was added TBAF (38.3 mL, 38.260 mmol, 1 mol/L in THF) slowly at room temperature. The resulting mixture was stirred for 2 h at room temperature. The reaction was quenched with water (30 mL). The mixture was extracted with EtOAc (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:1) to afford hex-5-yne-2,3-diol (1.50 g, 86%) as a yellow oil. MS ESI calculated for C₆H₁₀O₂[M+H]⁺, 115.07. found N/A. ¹H NMR (400 MHz, Chloroform-d) δ 4.01-3.73 (m, 1H), 3.76-3.48 (m, 1H), 2.53-2.34 (m, 2H), 2.08-2.04 (m, 1H), 1.22-1.15 (m, 3H).

### Preparation 243E: (7R,14R)-1-(difluoromethoxy)-11-(4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one

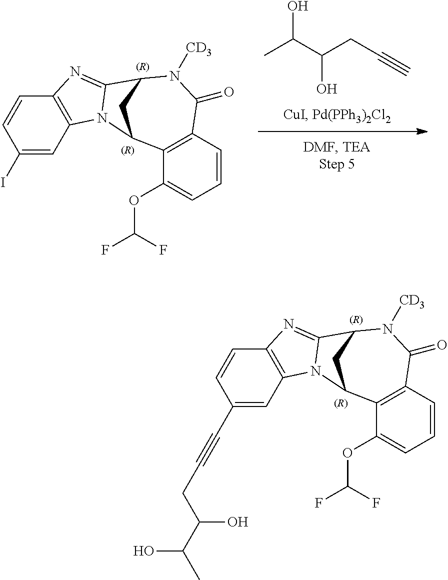

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (250 mg, 0.516 mmol) and hex-5-yne-2,3-diol (88 mg, 0.774 mmol) in TEA (2 mL) and DMF (2 mL) were added CuI (10 mg, 0.052 mmol), Pd(PPh₃)₂Cl₂ (36 mg, 0.052 mmol). The resulting mixture was stirred for 2 h at 80° C. under nitrogen atmosphere. The reaction was quenched with water (10 mL) at room temperature. The mixture was extracted with EtOAc (3×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 10% to 50% gradient in 10 min; detector, 254 nm. This resulted in (7R,14R)-1-(difluoromethoxy)-11-(4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one (210 mg, 86%) as a light yellow solid. MS ESI calculated for C₂₅H₂₀D₃F₂N₃O₄[M+H]⁺, 471.18. found 471.15. ¹H NMR (300 MHz, Chloroform-d) δ 8.38 (dd, J=7.5, 2.0 Hz, 1H), 7.67-7.44 (m, 4H), 7.46-6.97 (m, 2H), 6.36 (d, J=7.2 Hz, 1H), 5.18 (d, J=7.2 Hz, 1H), 3.98-3.77 (m, 1H), 3.71-3.47 (m, 2H), 2.88 (d, J=13.8 Hz, 1H), 2.84-2.55 (m, 2H), 1.33-1.21 (m, 3H).

### Example 243, 244, 245 and 246: (7R,14R)-1-(difluoromethoxy)-11-((4R or 4S,5S or 5R)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((4S or 4S,5R or 5S)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one; (7R,14R)-1-(difluoromethoxy)-11-((4S or 4R,5S or 5R)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one and (7R,14R)-1-(difluoromethoxy)-11-((4R or 4S,5R or 5S)-4,5-dihydroxyhex-1-yn-1-yl)-6-(methyl-d3)-6,7-dihydro-7,14-methanobenzo[f]benzo[4,5]imidazo[1,2-a][1,4]diazocin-5(14H)-one

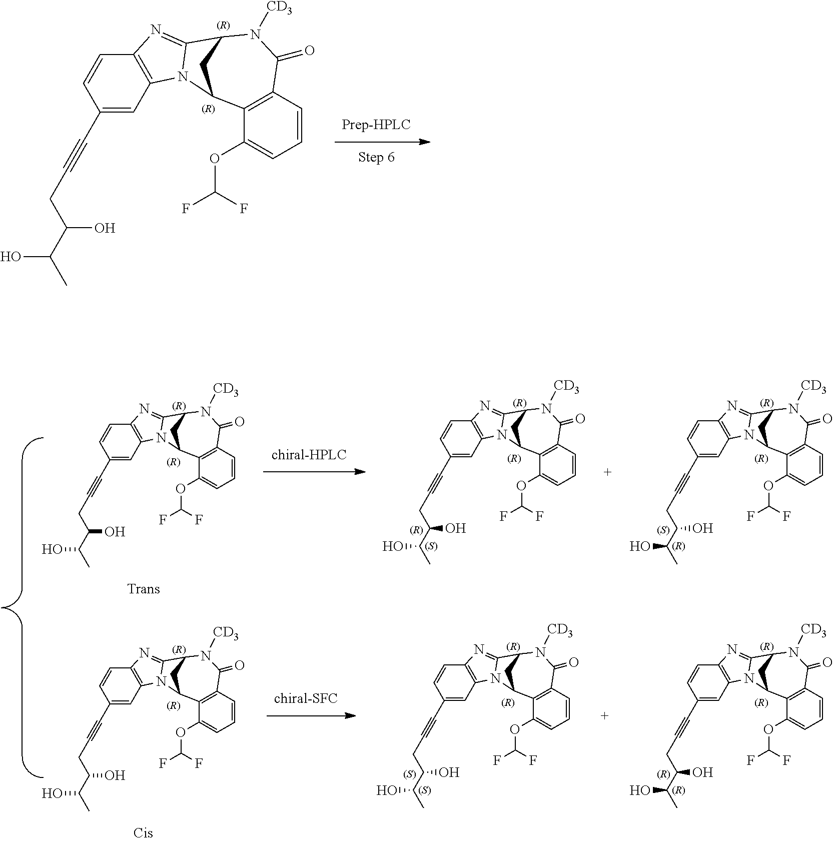

The mixture (210 mg) was purified by Prep-HPLC with the following conditions (Column: XBridge Prep OBD C18 Column 30*150 mm, 5m; Mobile Phase A: Water (10 mmol/L NH₄HCO₃), Mobile Phase B: MeOH; Flow rate: 60 mL/min; Gradient: 5% B to 5% B in 1.5 min, 5% B to 39% B in 2 min, 39% to 49% B in 11 min; Wave Length: 254 nm/220 nm; RT1(min): 10.33, RT1(min):11.42) to afford diastereoisomer I (40 mg) and diastereoisomer II (75 mg).

Then the diastereoisomer I was purified by chiral-HPLC with the following conditions (Column: CHIRALPAK IF, 2*25 cm, 5 μm; Mobile Phase A: Hex (10 mM NH₃-MeOH), Mobile Phase B: EtOH; Flow rate: 20 mL/min; Gradient: isocratic 30; Wave Length: 224/274 nm; RT1(min): 8.497; RT2(min): 10.198;). The first peak afforded (10 mg, 5%) as a white solid. MS ESI calculated for C₂₅H₂₀D₃F₂N₃O₄[M+H]⁺, 471.19 found 471.25. ¹H NMR (400 MHz, Methanol-d₄) δ 8.38 (dd, J=7.5, 2.0 Hz, 1H), 7.60-7.40 (m, 4H), 7.33-7.03 (m, 2H), 6.36 (d, J=7.2 Hz, 1H), 5.18 (d, J=7.2 Hz, 1H), 3.85-3.74 (m, 1H), 3.68-3.58 (m, 1H), 3.61-3.49 (m, 1H), 2.88 (d, J=13.8 Hz, 1H), 2.75 (dd, J=17.0, 4.8 Hz, 1H), 2.63 (dd, J=17.0, 6.9 Hz, 1H), 1.26 (d, J=6.4 Hz, 3H). ¹⁹F NMR (377 MHz, Methanol-d₄) δ −83.42. The second peak afforded (10 mg, 5%) as a white solid. MS ESI calculated for C₂₅H₂₀D₃F₂N₃O₄[M+H]⁺, 471.19 found 471.25. ¹H NMR (400 MHz, Methanol-d₄) δ 8.38 (dd, J=7.6, 2.0 Hz, 1H), 7.60-7.40 (m, 4H), 7.33-7.02 (m, 2H), 6.36 (d, J=7.1 Hz, 1H), 5.18 (d, J=7.1 Hz, 1H), 3.84-3.74 (m, 1H), 3.68-3.49 (m, 2H), 2.88 (d, J=13.9 Hz, 1H), 2.75 (dd, J=17.0, 4.8 Hz, 1H), 2.63 (dd, J=17.0, 7.0 Hz, 1H), 1.26 (d, J=6.3 Hz, 3H). ¹⁹F NMR (377 MHz, Methanol-d₄) δ −83.43.

The diastereoisomer II was purified by chiral-SFC with the following conditions (Column: NB-CHIRALCEL OD-H, 3*25 cm, 5 μm; Mobile Phase A: CO₂, Mobile Phase B: IPA:Hex=1:1 (20 mM NH₃); Flow rate: 100 mL/min; Gradient: isocratic 35% B; Wave Length: 224 nm; RT1(min): 15.55; RT2(min): 17.47). The first peak afforded (28 mg, 13%) as a white solid. MS ESI calculated for C₂₅H₂₀D₃F₂N₃O₄[M+H]⁺, 471.19 found 471.15. ¹H NMR (300 MHz, Methanol-d₄) δ 8.40 (dd, J=7.1, 2.4 Hz, 1H), 7.62-7.44 (m, 4H), 7.35-6.96 (m, 2H), 6.38 (d, J=7.2 Hz, 1H), 5.20 (d, J=7.1 Hz, 1H), 3.97-3.83 (m, 1H), 3.69-3.59 (m, 1H), 3.63-3.49 (m, 1H), 2.90 (d, J=13.9 Hz, 1H), 2.75 (dd, J=16.9, 5.9 Hz, 1H), 2.62 (dd, J=16.9, 6.6 Hz, 1H), 1.27 (d, J=6.4 Hz, 3H). ¹⁹F NMR (282 MHz, Methanol-d₄) δ −83.46. The second peak afforded (4 mg, 2%) as a white solid. MS ESI calculated for C₂₅H₂₀D₃F₂N₃O₄[M+H]⁺, 471.19 found 471.15. ¹H NMR (300 MHz, Methanol-d₄) δ 8.41 (dd, J=7.0, 2.5 Hz, 1H), 7.63-7.47 (m, 4H), 7.36-7.00 (m, 2H), 6.38 (d, J=7.2 Hz, 1H), 5.21 (d, J=7.2 Hz, 1H), 3.95-3.86 (m, 1H), 3.67-3.50 (m, 2H), 2.91 (d, J=13.9 Hz, 1H), 2.75 (dd, J=16.9, 5.9 Hz, 1H), 2.62 (dd, J=16.9, 6.6 Hz, 1H), 1.27 (d, J=6.5 Hz, 3H). ¹⁹F NMR (282 MHz, Methanol-d₄) δ −83.47.

### Example 247: (7R,14R)-1-(difluoromethoxy)-11-(3,3-difluoroprop-1-yn-1-yl)-6-(methyl-d3)-6,7-dihydro-7,14-methanobenzo[f]benzo[4,5]imidazo[1,2-a][1,4]diazocin-5(14H)-one

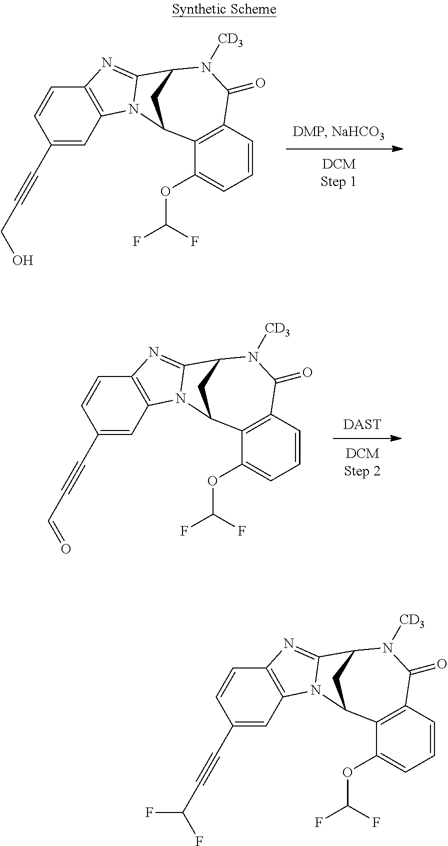

### Preparation 247A: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolaldehyde

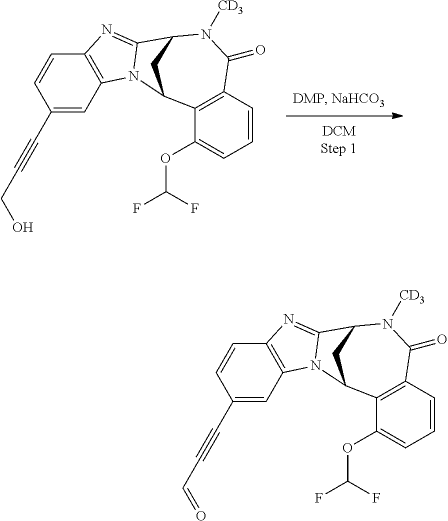 [SMILES: [2H]C([2H])([2H])N1C(=O)c2cccc(OC(F)F)c2[C@H]2C[C@@H]1c1nc3ccc(C#CCO)cc3n12 ; [2H]C([2H])([2H])N1C(=O)c2cccc(OC(F)F)c2[C@H]2C[C@@H]1c1nc3ccc(C#CC=O)cc3n12]

To a stirred solution of (7R,14R)-1-(difluoromethoxy)-11-(3-hydroxyprop-1-yn-1-yl)-6-(methyl-d3)-6,7-dihydro-7,14-methanobenzo[f]benzo[4,5]imidazo[1,2-a][1,4]diazocin-5(14H)-one (280 mg, 0.679 mmol) and NaHCO₃ (570 mg, 6.790 mmol) in DCM (60 mL) was added DMP (576 mg, 1.358 mmol). The resulting mixture was stirred for 16 h at room temperature. The mixture was filtered, the filter cake was washed with CH₂Cl₂ (3×20 mL). The filtrate was concentrated under reduced pressure. The residue was purified by silica gel column chromatography, eluted with CH₂Cl₂/MeOH (10/1) to afford 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolaldehyde (260 mg, 93%) as a yellow solid. MS ESI calculated for C₂₂H₁₂D₃F₂N₃O₃ [M+H]⁺, 411.13 found 411.30. ¹H NMR (400 MHz, Chloroform-d) δ 9.44 (s, 1H), 8.49 (d, J=8.1 Hz, 1H), 7.74-7.71 (m, 2H), 7.50-7.40 (m, 2H), 7.37 (d, J=8.1 Hz, 1H), 6.86 (t, J=72.6 Hz, 1H), 6.25 (d, J=7.2 Hz, 1H), 5.02 (d, J=7.1 Hz, 1H), 3.52-3.41 (m, 1H), 2.89 (d, J=13.6 Hz, 1H). ¹⁹F NMR (377 MHz, Chloroform-d) δ −80.14, −80.59, −81.11, −81.55.

### Example 247: (7R,14R)-1-(difluoromethoxy)-11-(3,3-difluoroprop-1-yn-1-yl)-6-(methyl-d3)-6,7-dihydro-7,14-methanobenzo[f]benzo[4,5]imidazo[1,2-a][1,4]diazocin-5(14H)-one

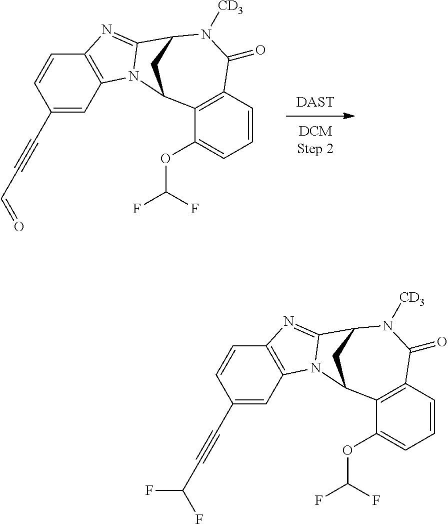

To a stirred solution of 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolaldehyde (60 mg, 0.146 mmol) in DCM (1 mL) was added DAST (71 mg, 0.438 mmol) dropwise at room temperature. The resulting mixture was stirred for 16 h at room temperature. The mixture was concentrated under reduced pressure and purified by silica gel column chromatography, eluted with DCM/MeOH (0% to 10%), followed by Prep-HPLC with the following conditions: Column: C18 Column 120 g; Mobile Phase A: water (10 mmol/L NH₄HCO₃), Mobile Phase B: CH₃CN; Flow rate: 60 mL/min; Gradient: 30% B to 70% B in 20 min; 254/220 nm to afford (7R,14R)-1-(difluoromethoxy)-11-(3,3-difluoroprop-1-yn-1-yl)-6-(methyl-d3)-6,7-dihydro-7,14-methanobenzo[f]benzo[4,5]imidazo[1,2-a][1,4]diazocin-5(14H)-one (16 mg, 25%) as a white solid. MS ESI calculated for C₂₂H₁₂D₃F₄N₃O₂ [M+H]⁺, 433.13 found 433.00. ¹H NMR (400 MHz, Chloroform-d) δ 8.52-8.46 (m, 1H), 7.69 (d, J=8.4 Hz, 1H), 7.63 (s, 1H), 7.48-7.41 (m, 1H), 7.41-7.34 (m, 2H), 7.07-6.65 (m, 1H), 6.44 (t, J=55.2 Hz, 1H), 6.23 (d, J=7.2 Hz, 1H), 4.96 (d, J=7.1 Hz, 1H), 3.52-3.39 (m, 1H), 2.88 (d, J=13.6 Hz, 1H). ¹⁹F NMR (377 MHz, Chloroform-d) δ −80.06, −80.50, −81.18, −81.62, −105.03.

### Example 248: (7R,14R)-11-(3-(cyclopropylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

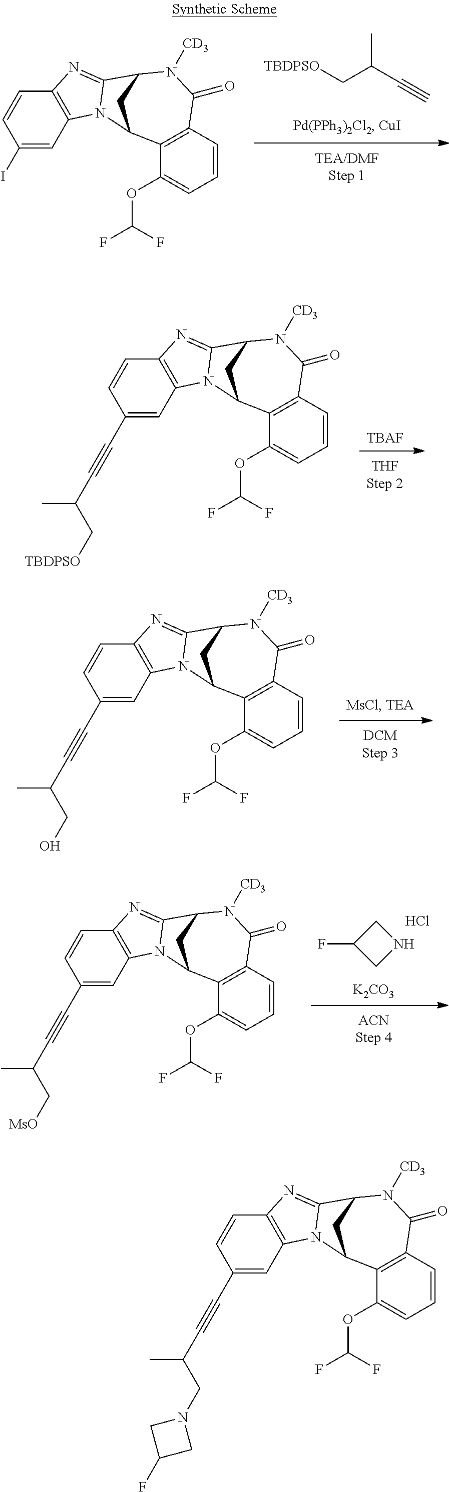

### Preparation 248A: (7R,14R)-11-(4-((tert-butyldiphenylsilyl)oxy)-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

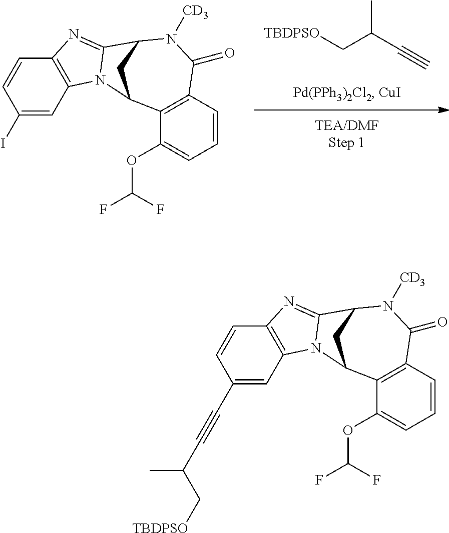

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (300 mg, 0.620 mmol) and tert-butyl[(2-methylbut-3-yn-1-yl)oxy]diphenylsilane (300 mg, 0.930 mmol) in TEA (1.5 mL) and DMF (1.5 mL) were added Pd(PPh₃)₂Cl₂ (44 mg, 0.062 mmol) and CuI (12 mg, 0.062 mmol). The resulting mixture was stirred for 2 h at 80° C. under nitrogen atmosphere. The mixture was concentrated under reduced pressure. The residue was purified by silica gel column chromatography, eluted with CH₂Cl₂/MeOH (12/1) to afford (7R,14R)-11-(4-((tert-butyldiphenylsilyl)oxy)-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (390 mg, 93%) as a yellow solid. MS ESI calculated for C₄₀H₃₆D₃F₂N₃O₃Si [M+H]⁺, 679.29. found 679.35. ¹H NMR (400 MHz, Chloroform-d) δ 8.55-8.43 (m, 1H), 7.77-7.64 (m, 4H), 7.58 (d, J=8.5 Hz, 1H), 7.49-7.45 (m, 1H), 7.44-7.42 (m, 1H), 7.41-7.34 (m, 6H), 7.34-7.28 (m, 2H), 6.71 (t, J=73.3 Hz, 1H), 6.18 (d, J=7.2 Hz, 1H), 4.96 (d, J=7.1 Hz, 1H), 3.86-3.77 (m, 1H), 3.69-3.61 (m, 1H), 3.51-3.36 (m, 1H), 2.95-2.80 (m, 2H), 1.34-1.30 (m, 3H), 1.08 (s, 9H). ¹⁹F NMR (377 MHz, Chloroform-d) δ −80.00, −80.02, −80.44, −80.46, −81.14, −81.14, −81.58, −81.59.

### Preparation 248B: (7R,14R)-1-(difluoromethoxy)-11-(4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one

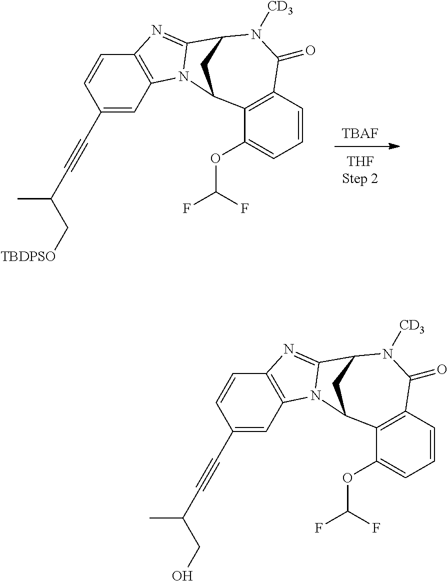

To a stirred solution of (7R,14R)-11-(4-((tert-butyldiphenylsilyl)oxy)-3-methylbut-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one (390 mg, 0.574 mmol) in THF (4 mL) was added TBAF (0.69 mL, 0.689 mmol) at 0° C. The resulting mixture was stirred for 1 h at room temperature. The mixture was concentrated under vacuum. The residue was purified by silica gel column chromatography, eluted with CH₂Cl₂/MeOH (12/1) to afford (7R,14R)-1-(difluoromethoxy)-11-(4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (200 mg, 79%) as a yellow solid. MS ESI calculated for C₂₄H₁₈D₃F₂N₃O₃[M+H]⁺, 441.17. found 441.15. ¹H NMR (400 MHz, Chloroform-d) δ 8.48 (dd, J=8.2, 1.3 Hz, 1H), 7.61 (d, J=8.5 Hz, 1H), 7.51 (s, 1H), 7.45-7.39 (m, 1H), 7.36-7.31 (m, 1H), 7.30-7.27 (m, 1H), 7.05-6.64 (m, 1H), 6.21 (d, J=7.2 Hz, 1H), 4.96 (d, J=7.1 Hz, 1H), 3.75-3.60 (m, 2H), 3.50-3.37 (m, 1H), 2.97-2.89 (m, 1H), 2.85 (d, J=13.6 Hz, 1H), 1.28 (d, J=6.9 Hz, 3H). ¹⁹F NMR (377 MHz, Chloroform-d) δ −80.00, −80.01, −80.44, −80.45, −81.25, −81.27, −81.69, −81.71.

### Preparation 248C: 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-1-yl Methanesulfonate

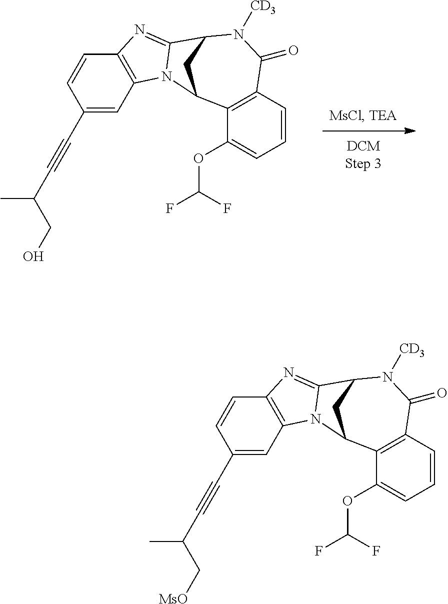 [SMILES: [2H]C([2H])([2H])N1C(=O)c2cccc(OC(F)F)c2[C@H]2C[C@@H]1c1nc3ccc(C#CC(C)CO)cc3n12 ; "MsCl, TEA" ; [2H]C([2H])([2H])N1C(=O)c2cccc(OC(F)F)c2[C@H]2C[C@@H]1c1nc3ccc(C#CC(C)COS(C)(=O)=O)cc3n12]

To a stirred solution of (7R,14R)-1-(difluoromethoxy)-11-(4-hydroxy-3-methylbut-1-yn-1-yl)-6-(methyl-d3)-6,7-dihydro-7,14-methanobenzo[f]benzo[4,5]imidazo[1,2-a][1,4]diazocin-5(14H)-one (180 mg, 0.409 mmol) and TEA (62 mg, 0.613 mmol) in DCM (3 mL) were added MsCl (38 uL, 0.491 mmol) at 0° C. The resulting mixture was stirred for 1 h at room temperature under nitrogen atmosphere. The reaction was quenched with water. The mixture was extracted with CH₂Cl₂ (2×10 mL). The combined organic layers were washed with brine (1×10 mL), dried over anhydrous Na₂SO₄. After filtration, the filtrate was concentrated under reduced pressure to afford 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-1-yl methanesulfonate (200 mg, 94%) as a yellow solid. MS ESI calculated for C₂₅H₂₀D₃F₂N₃O₅S [M+H]⁺, 519.15. found 519.15. ¹H NMR (300 MHz, Chloroform-d) δ 8.50 (dd, J=8.1, 1.4 Hz, 1H), 7.65 (d, J=8.5 Hz, 1H), 7.53 (s, 1H), 7.45 (t, J=8.2 Hz, 1H), 7.41-7.29 (m, 2H), 7.17-6.62 (m, 1H), 6.23 (d, J=7.2 Hz, 1H), 4.97 (d, J=7.1 Hz, 1H), 4.40-4.28 (m, 1H), 4.28-4.16 (m, 1H), 3.54-3.40 (m, 1H), 3.22-3.10 (m, 1H), 3.09 (s, 3H), 2.88 (d, J=13.6 Hz, 1H), 1.39 (d, J=6.9 Hz, 3H). ¹⁹F NMR (282 MHz, Chloroform-d) δ −79.91, −79.92, −80.50, −80.51, −81.13, −81.16, −81.73, −81.75.

### Example 248: (7R,14R)-11-(3-(cyclopropylamino)prop-1-yn-1-yl)-1-(difluoromethoxy)-6-(methyl-d3)-6,7-dihydro-7,14-methanobenzo[f]benzo[4,5]imidazo[1,2-a][1,4]diazocin-5(14H)-one

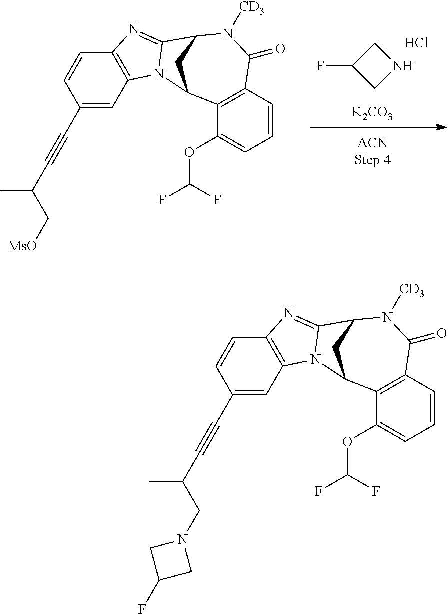

To a stirred solution of 4-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)-2-methylbut-3-yn-1-yl methanesulfonate (100 mg, 0.193 mmol) and 3-fluoroazetidine hydrochloride (215 mg, 1.930 mmol) in ACN (1 mL) was added K₂CO₃ (133 mg, 0.965 mmol) at room temperature. The resulting mixture was stirred for overnight at 80° C. The reaction was quenched with water (10 mL). The mixture was extracted with EtOAc (2×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (10 mmol/L NH₄HCO₃), 30% to 50% gradient in 20 min; detector, 254 nm followed by reversed-phase flash chromatography with the following conditions: column, C18 silica gel; mobile phase, CH₃CN in Water (0.1% FA), 20% to 40% gradient in 20 min; detector, 254/220 nm. This resulted in (7R,14R)-1-(difluoromethoxy)-11-(4-(3-fluoroazetidin-1-yl)-3-methylbut-1-yn-1-yl)-6-(methyl-d3)-6,7-dihydro-7,14-methanobenzo[f]benzo[4,5]imidazo[1,2-a][1,4]diazocin-5(14H)-one (5 mg, 5%) as a yellow solid. MS ESI calculated for C₂₇H₂₂D₃F₃N₄O₂[M+H]⁺, 498.21. found 498.25. ¹H NMR (400 MHz, Chloroform-d) δ 8.48 (d, J=8.2 Hz, 1H), 7.60 (d, J=8.4 Hz, 1H), 7.49 (s, 1H), 7.46-7.38 (m, 1H), 7.35-7.30 (m, 1H), 7.26-7.22 (m, 1H), 6.85 (t, J=72.8 Hz, 1H), 6.20 (d, J=7.2 Hz, 1H), 5.29-5.05 (m, 1H), 4.94 (d, J=7.1 Hz, 1H), 3.99-3.82 (m, 1H), 3.53-3.26 (m, 4H), 3.01-2.62 (m, 4H), 1.26 (d, J=6.7 Hz, 3H). ¹⁹F NMR (377 MHz, Chloroform-d) δ −80.04, −80.49, −81.24, −81.25, −81.69, −81.70, −177.41, −177.47.

### Example 249: (7R,14R)-11-((2-aminopyrimidin-5-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

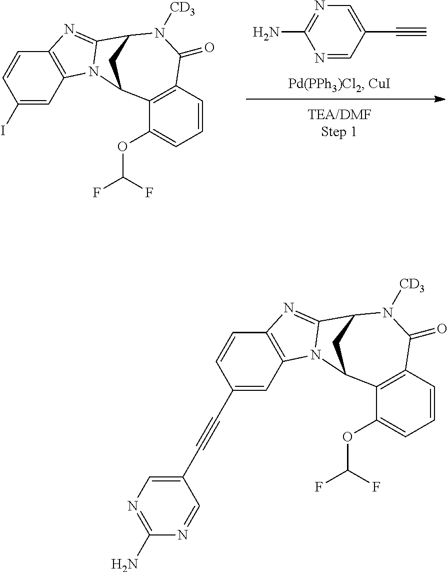

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (66 mg, 0.558 mmol) in TEA (1.5 mL) and DMF (1.5 mL) were added Pd(PPh₃)2C₂ (26 mg, 0.037 mmol) and CuI (7 mg, 0.037 mmol) at room temperature. The resulting mixture was stirred for 2 h at 80° C. under nitrogen atmosphere. The mixture was allowed to cool down to room temperature, diluted with water (10 mL), and extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with CH₂Cl₂/MeOH (12:1) followed by reversed-phase flash chromatography with the following conditions: column, C18 silica gel; mobile phase, CH₃CN in Water (10 mmol/L NH₄HCO₃), 10% to 36% gradient in 20 min; detector, 254 nm to afford (7R,14R)-11-((2-aminopyrimidin-5-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (13 mg, 7%) as a white solid. MS ESI calculated for C₂₅H₁₅D₃F₂N₆O₂[M+H]⁺, 476.17 found 476.10. ¹H NMR (400 MHz, Chloroform-d) δ 8.57-8.42 (m, 3H), 7.99-7.52 (m, 2H), 7.48-7.31 (m, 3H), 6.85 (t, J=72.7 Hz, 1H), 6.24 (d, J=7.0 Hz, 1H), 5.34 (s, 2H), 4.98 (s, 1H), 3.67-3.30 (m, 1H), 2.87 (d, J=13.5 Hz, 1H). ¹⁹F NMR (377 MHz, Chloroform-d) δ −79.97, −80.41, −81.05, −81.50.

### Example 250: (7R,14R)-1-(difluoromethoxy)-11-((3-hydroxy-1-(oxetan-3-yl)azetidin-3-yl)ethynyl)-6-(methyl-d3)-6,7-dihydro-7,14-methanobenzo[f]benzo[4,5]imidazo[1,2-a][1,4]diazocin-5(14H)-one

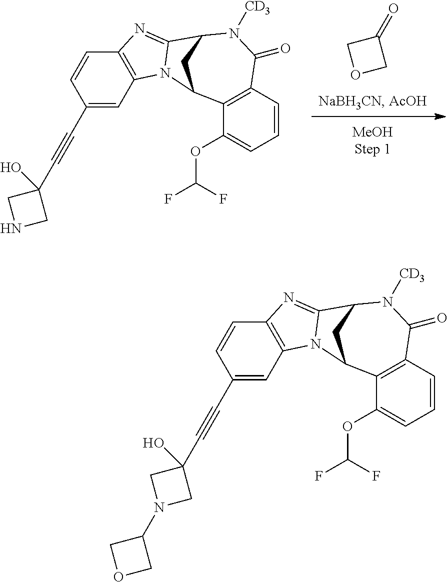

To a solution of (7R,14R)-1-(difluoromethoxy)-11-((3-hydroxyazetidin-3-yl)ethynyl)-6-(methyl-d3)-6,7-dihydro-7,14-methanobenzo[f]benzo[4,5]imidazo[1,2-a][1,4]diazocin-5(14H)-one (40 mg, 0.088 mmol), 3-oxetanone (32 mg, 0.440 mmol) and ACOH (16 mg, 0.264 mmol) in MeOH (0.5 mL) was added NaBH₃CN (17 mg, 0.264 mmol). The reaction was stirred for 1 h at room temperature. The mixture was concentrated under reduced pressure and purified by reversed-phase flash chromatography with the following conditions: column, C18 silica gel; mobile phase, CH₃CN in Water (10 mmol/L NH₄HCO₃), 40% to 50% gradient in 10 min; detector, 254 nm. The crude product was purified by Prep-HPLC with the following conditions (Column: XBridge Shield RP18 OBD Column 30*150 mm, 5m; Mobile Phase A: Water (10 mmol/L NH₄HCO₃+0.05% NH₃·H₂O), Mobile Phase B: CH₃CN; Flow rate: 60 mL/min; Gradient: 15% B to 30% B in 12 min; Wave Length: 254/220 nm; RT1(min): 12.57) to afford (7R,14R)-1-(difluoromethoxy)-11-((3-hydroxy-1-(oxetan-3-yl)azetidin-3-yl)ethynyl)-6-(methyl-d3)-6,7-dihydro-7,14-methanobenzo[f]benzo[4,5]imidazo[1,2-a][1,4]diazocin-5(14H)-one (5 mg, 12%) as a white solid. MS ESI calculated for C₂₇H₂₁D₃F₂N₄O₄ [M+H]⁺, 510.20. found 510.15. ¹H NMR (400 MHz, Methanol-d₄) δ 8.37 (dd, J=7.4, 2.1 Hz, 1H), 7.67-7.61 (m, 1H), 7.59 (d, J=8.4 Hz, 1H), 7.53-7.42 (m, 2H), 7.43-7.02 (m, 2H), 6.36 (d, J=7.2 Hz, 1H), 5.18 (d, J=7.1 Hz, 1H), 4.74 (t, J=6.8 Hz, 2H), 4.53 (dd, J=6.9, 5.1 Hz, 2H), 3.96-3.85 (m, 1H), 3.80-3.72 (m, 2H), 3.61-3.49 (m, 1H), 3.50-3.41 (m, 2H), 2.88 (d, J=13.8 Hz, 1H). ¹⁹F NMR (377 MHz, Methanol-d₄) δ −82.94, −83.39, −83.41, −83.86.

### Example 251: (7R,14R)-1-(difluoromethoxy)-11-((3-hydroxyazetidin-3-yl)ethynyl)-6-(methyl-d3)-6,7-dihydro-7,14-methanobenzo[f]benzo[4,5]imidazo[1,2-a][1,4]diazocin-5(14H)-one

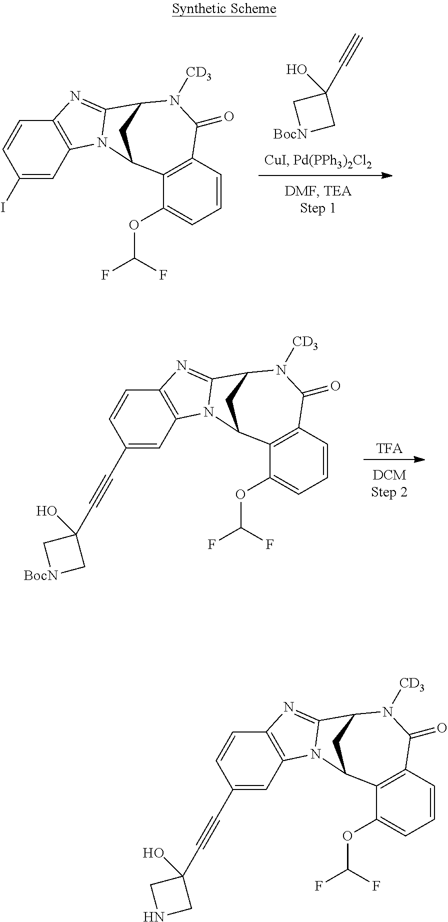

### Preparation 251A: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-hydroxyazetidine-1-carboxylate

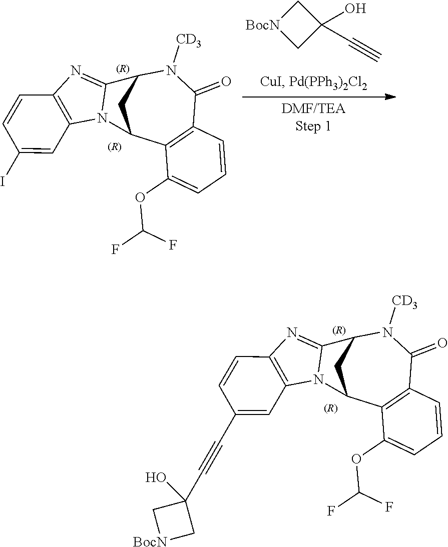

A mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (100 mg, 0.207 mmol), tert-butyl 3-ethynyl-3-hydroxyazetidine-1-carboxylate (53 mg, 0.269 mmol), CuI (4 mg, 0.021 mmol) and Pd(PPh₃)2C₂ (14 mg, 0.021 mmol) in DMF (1 mL) and TEA (1 mL) was stirred for 1 h at 80° C. under nitrogen atmosphere. The mixture was concentrated under reduced pressure. The residue was purified by silica gel column chromatography, eluted with DCM/MeOH (10/1) followed by reversed-phase flash chromatography with the following conditions: column, C18 silica gel; mobile phase, CH₃CN in Water (10 mmol/L NH₄HCO₃), 40% to 50% gradient in 10 min; detector, 254 nm. This resulted in tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-hydroxyazetidine-1-carboxylate (91 mg, 80%) as a light yellow solid. MS ESI calculated for C₂₉H₂₅D₃F₂N₄O₅ [M+H]⁺, 554.22 found 554.20. ¹H NMR (400 MHz, Chloroform-d) δ 8.44 (dd, J=8.1, 1.4 Hz, 1H), 7.60 (d, J=8.4 Hz, 1H), 7.52-7.44 (m, 1H), 7.38 (t, J=8.2 Hz, 1H), 7.31 (d, J=8.1 Hz, 1H), 7.17 (dd, J=8.4, 1.5 Hz, 1H), 6.84 (t, J=72.2 Hz, 1H), 6.18 (d, J=7.2 Hz, 1H), 4.93 (d, J=7.1 Hz, 1H), 4.31 (d, J=9.0 Hz, 2H), 4.13 (d, J=9.0 Hz, 2H), 3.67 (s, 1H), 3.48-3.35 (m, 1H), 2.83 (d, J=13.6 Hz, 1H), 1.45 (s, 9H). ¹⁹F NMR (377 MHz, Chloroform-d) δ −80.07, −80.51, −81.24, −81.69.

### Example 251: (7R,14R)-1-(difluoromethoxy)-11-((3-hydroxyazetidin-3-yl)ethynyl)-6-(methyl-d3)-6,7-dihydro-7,14-methanobenzo[f]benzo[4,5]imidazo[1,2-a][1,4]diazocin-5(14H)-one

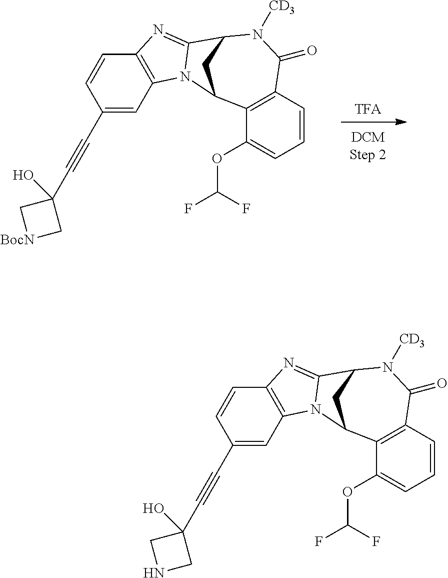 [SMILES: [2H]C([2H])([2H])N1C(=O)c2cccc(OC(F)F)c2[C@H]2C[C@@H]1c1nc3ccc(C#CC4(O)CN(C(=O)OC(C)(C)C)C4)cc3n12 ; [2H]C([2H])([2H])N1C(=O)c2cccc(OC(F)F)c2[C@H]2C[C@@H]1c1nc3ccc(C#CC4(O)CNC4)cc3n12 ; "TFA"]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-hydroxyazetidine-1-carboxylate (80 mg, 0.145 mmol) in DCM (2 mL) was added TFA (0.4 mL). The resulting mixture was stirred for 1 h at room temperature. The mixture was concentrated under reduced pressure. The residue was purified by reversed-phase flash chromatography with the following conditions: column, C18 silica gel; mobile phase, CH₃CN in Water (10 mmol/L NH₄HCO₃), 30% to 40% gradient in 10 min; detector, 254 nm. This resulted in (7R,14R)-1-(difluoromethoxy)-11-((3-hydroxyazetidin-3-yl)ethynyl)-6-(methyl-d3)-6,7-dihydro-7,14-methanobenzo[f]benzo[4,5]imidazo[1,2-a][1,4]diazocin-5(14H)-one (60 mg, 92%) as a white solid. MS ESI calculated for C₂₄H₁₇D₃F₂N₄O₃ [M+H]⁺, 454.17. found 454.00. ¹H NMR (400 MHz, DMSO-d) S 8.28 (d, J=7.5 Hz, 1H), 7.78-7.56 (m, 2H), 7.57-7.37 (m, 3H), 7.25 (d, J=8.4 Hz, 1H), 6.23 (d, J=7.0 Hz, 2H), 5.23 (d, J=7.1 Hz, 1H), 4.18-3.59 (m, 4H), 3.57-3.43 (m, 2H), 2.81 (d, J=13.8 Hz, 1H). ¹⁹F NMR (377 MHz, DMSO-d₆) δ −81.96.

### Example 252: (7R,14R)-1-(difluoromethoxy)-11-((3-fluoro-1-(oxetan-3-yl)azetidin-3-yl)ethynyl)-6-(methyl-d3)-6,7-dihydro-7,14-methanobenzo[f]benzo[4,5]imidazo[1,2-a][1,4]diazocin-5(14H)-one

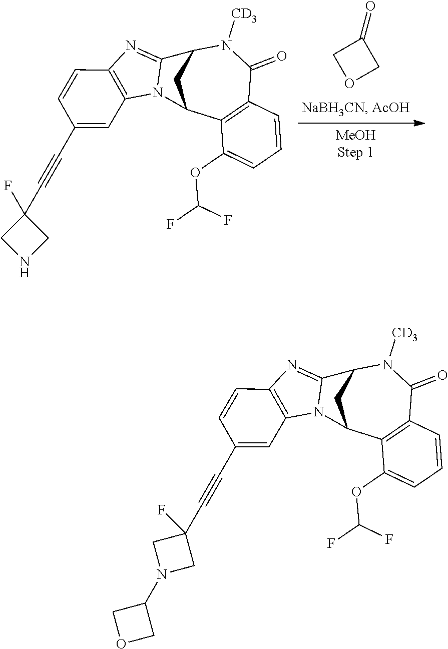 [SMILES: [2H]C([2H])([2H])N1C(=O)c2cccc(OC(F)F)c2[C@H]2C[C@@H]1c1nc3ccc(C#CC4(F)CNC4)cc3n12 ; O=C1COC1 ; [2H]C([2H])([2H])N1C(=O)c2cccc(OC(F)F)c2[C@H]2C[C@@H]1c1nc3ccc(C#CC4(F)CN(C5COC5)C4)cc3n12]

To a stirred solution of (7R,14R)-1-(difluoromethoxy)-11-((3-fluoroazetidin-3-yl)ethynyl)-6-(methyl-d3)-6,7-dihydro-7,14-methanobenzo[f]benzo[4,5]imidazo[1,2-a][1,4]diazocin-5(14H)-one (40 mg, 0.088 mmol) in MeOH (1 mL) was added 3-oxetanone (31 mg, 0.440 mmol). The resulting mixture was stirred for 15 min at room temperature. To the above mixture was added NaBH₃CN (8 mg, 0.132 mmol). The reaction was stirred for additional 3 h at room temperature. The mixture was concentrated under reduced pressure. The residue was purified by reversed-phase flash chromatography with the following conditions: column, C18 silica gel; mobile phase, CH₃CN in Water (10 mmol/L NH₄HCO₃), 10% to 40% gradient in 10 min; detector, 254 nm to afford (7R,14R)-1-(difluoromethoxy)-11-((3-fluoro-1-(oxetan-3-yl)azetidin-3-yl)ethynyl)-6-(methyl-d3)-6,7-dihydro-7,14-methanobenzo[f]benzo[4,5]imidazo[1,2-a][1,4]diazocin-5(14H)-one (4 mg, 10%) as a white solid. MS ESI calculated for C₂₇H₂₀D₃F₃N₄O₃[M+H]⁺, 512.19. found 512.05. ¹H NMR (400 MHz, Chloroform-d) δ 8.49 (dd, J=8.2, 1.3 Hz, 1H), 7.66 (d, J=8.4 Hz, 1H), 7.59 (s, 1H), 7.44 (t, J=8.2 Hz, 1H), 7.39-7.32 (m, 2H), 7.07-6.65 (m, 1H), 6.22 (d, J=7.2 Hz, 1H), 4.96 (d, J=7.1 Hz, 1H), 4.77 (t, J=6.9 Hz, 2H), 4.64-4.56 (m, 2H), 4.05-3.88 (m, 3H), 3.86-3.75 (m, 2H), 3.51-3.40 (m, 1H), 2.87 (d, J=13.6 Hz, 1H). ¹⁹F NMR (377 MHz, Chloroform-d) δ −80.02, −80.46, −81.25, −81.69, −137.72.

### Example 253: (7R,14R)-1-(difluoromethoxy)-11-((3-fluoroazetidin-3-yl)ethynyl)-6-(methyl-d3)-6,7-dihydro-7,14-methanobenzo[f]benzo[4,5]imidazo[1,2-a][1,4]diazocin-5(14H)-one

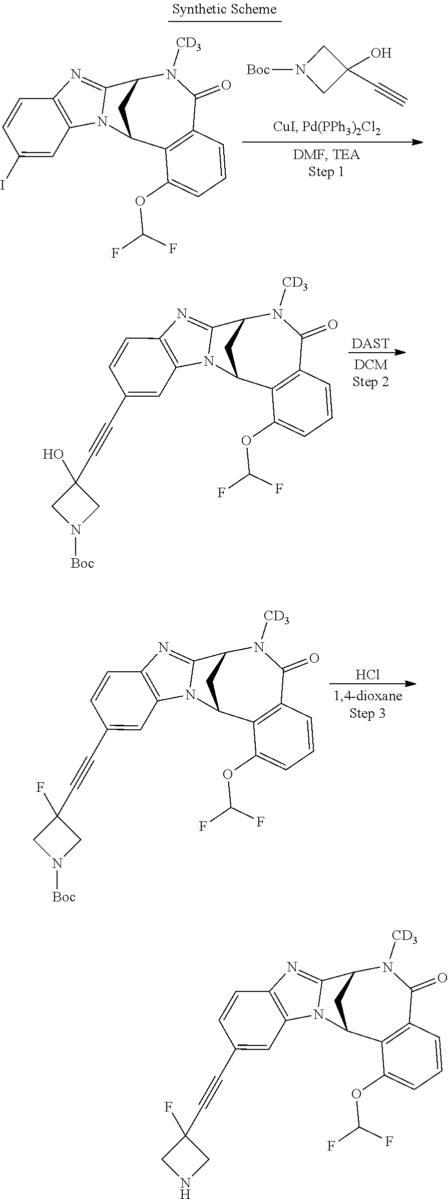

### Preparation 253A: (7R,14R)-1-(difluoromethoxy)-11-((3-fluoroazetidin-3-yl)ethynyl)-6-(methyl-d3)-6,7-dihydro-7,14-methanobenzo[f]benzo[4,5]imidazo[1,2-a][1,4]diazocin-5(14H)-one

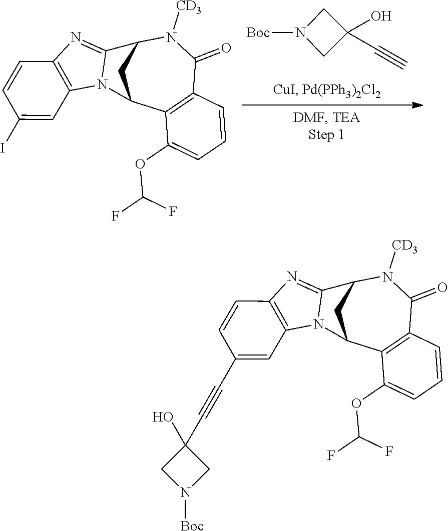

To a stirred solution of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (200 mg, 0.413 mmol), CuI (7 mg, 0.041 mmol) and tert-butyl 3-ethynyl-3-hydroxyazetidine-1-carboxylate (106 mg, 0.537 mmol) in DMF (3 mL) and TEA (3 mL) was added Pd(PPh₃)₂Cl₂ (28 mg, 0.041 mmol). The resulting mixture was stirred for 1 h at 80° C. under nitrogen atmosphere. The mixture was allowed to cool down to room temperature and concentrated under vacuum. The residue was purified by silica gel column chromatography, eluted with DCM/MeOH (9:1) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-hydroxyazetidine-1-carboxylate (210 mg, 91%) as a yellow solid. MS ESI calculated for C9H₂₅D₃F₂N₄O₅ [M+H]⁺, 554.22 found 554.15. ¹H NMR (400 MHz, Chloroform-d) δ 8.44 (dd, J=8.1, 1.4 Hz, 1H), 7.60 (d, J=8.4 Hz, 1H), 7.49 (s, 1H), 7.38 (t, J=8.2 Hz, 1H), 7.31 (d, J=8.1 Hz, 1H), 7.17 (dd, J=8.4, 1.5 Hz, 1H), 7.05-6.62 (m, 1H), 6.18 (d, J=7.2 Hz, 1H), 4.94 (d, J=7.1 Hz, 1H), 4.31 (d, J=9.0 Hz, 2H), 4.13 (d, J=9.0 Hz, 2H), 3.67 (s, 1H), 3.51-3.28 (m, 1H), 2.83 (d, J=13.6 Hz, 1H), 1.46 (s, 9H).

### Preparation 253B: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-fluoroazetidine-1-carboxylate

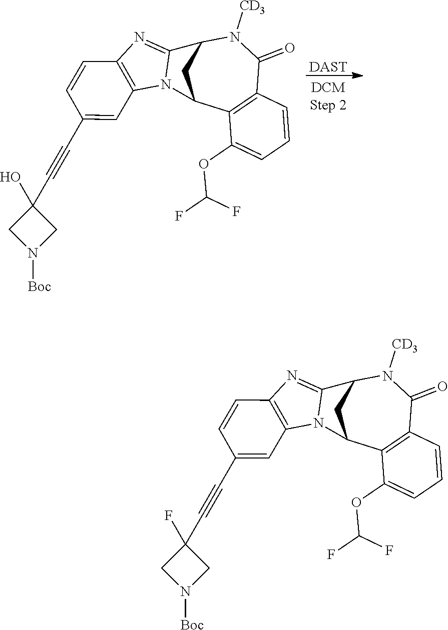

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-hydroxyazetidine-1-carboxylate (210 mg, 0.379 mmol) in DCM (3 mL) was added DAST (122 mg, 0.758 mmol) dropwise at −78° C. The resulting mixture was stirred for 1 h at −78° C. and stirred for 1 h at room temperature. The reaction was quenched by the addition of water (10 mL). The mixture was extracted with CH₂Cl₂ (3×10 mL). The combined organic layers were washed with brine (10 mL), dried over anhydrous Na₂SO₄, filtered and concentrated under reduced pressure and purified by silica gel column chromatography, eluted with CH₂Cl₂/MeOH (12:1) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-fluoroazetidine-1-carboxylate (110 mg, 52%) as a yellow solid. MS ESI calculated for C9H₂₄D₃F₃N₄O₄ [M+H]⁺, 556.22. found 556.20. ¹H NMR (400 MHz, Chloroform-d) δ 8.49 (dd, J=8.2, 1.3 Hz, 1H), 7.72-7.63 (m, 1H), 7.60-7.54 (m, 1H), 7.43 (t, J=8.2 Hz, 1H), 7.39-7.31 (m, 2H), 7.05-6.64 (m, 1H), 6.23 (d, J=7.1 Hz, 1H), 4.96 (d, J=7.0 Hz, 1H), 4.38-4.29 (m, 4H), 3.55-3.38 (m, 1H), 2.87 (d, J=13.5 Hz, 1H), 1.47 (s, 9H).

### Example 253: (7R,14R)-1-(difluoromethoxy)-11-((3-fluoroazetidin-3-yl)ethynyl)-6-(methyl-d3)-6,7-dihydro-7,14-methanobenzo[f]benzo[4,5]imidazo[1,2-a][1,4]diazocin-5(14H)-one

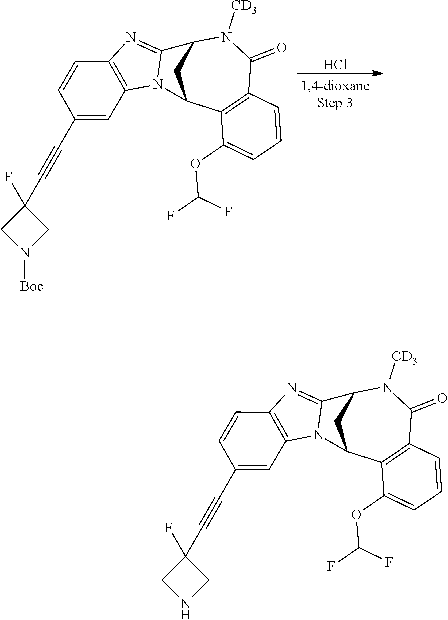 [SMILES: [2H]C([2H])([2H])N1C(=O)c2cccc(OC(F)F)c2[C@H]2C[C@@H]1c1nc3ccc(C#CC4(F)CN(C(=O)OC(C)(C)C)C4)cc3n12 ; Cl ; [2H]C([2H])([2H])N1C(=O)c2cccc(OC(F)F)c2[C@H]2C[C@@H]1c1nc3ccc(C#CC4(F)CNC4)cc3n12]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-3-fluoroazetidine-1-carboxylate (20 mg, 0.036 mmol) in 1,4-dioxane (0.1 mL) was added HCl (0.1 mL, 0.4 mmol, 4 M in 1,4-dioxane) at room temperature. The resulting mixture was stirred for 2 h at room temperature. The mixture was concentrated under reduced pressure. The residue was purified by reversed-phase flash chromatography with the following conditions: column, C18 silica gel; mobile phase, CH₃CN in Water (10 mmol/L NH₄HCO₃), 10% to 40% gradient in 10 min; detector, 254 nm to afford (7R,14R)-1-(difluoromethoxy)-11-((3-fluoroazetidin-3-yl)ethynyl)-6-(methyl-d3)-6,7-dihydro-7,14-methanobenzo[f]benzo[4,5]imidazo[1,2-a][1,4]diazocin-5(14H)-one (9 mg, 58%) as a white solid. MS ESI calculated for C₂₄H₁₆D₃F₃N₄O₂ [M+H]⁺, 456.16. found 456.05. 1H NMR (400 MHz, Chloroform-d) δ 8.48 (dd, J=8.2, 1.3 Hz, 1H), 7.66 (d, J=8.4 Hz, 1H), 7.58 (s, 1H), 7.43 (t, J=8.1 Hz, 1H), 7.39-7.31 (m, 2H), 6.87 (t, J=73.2 Hz, 1H), 6.22 (d, J=7.2 Hz, 1H), 4.95 (d, J=7.1 Hz, 1H), 4.44-3.96 (m, 4H), 3.51-3.39 (m, 1H), 2.87 (d, J=13.6 Hz, 1H). ¹⁹F NMR (377 MHz, Chloroform-d) δ −80.01, −80.45, −81.33, −81.78, −134.86.

### Example 254: (7R,14R)-1-(difluoromethoxy)-6-(methyl-d3)-11-(((1r,3R)-3-(methylamino)cyclobutyl)ethynyl)-6,7-dihydro-7,14-methanobenzo[f]benzo[4,5]imidazo[1,2-a][1,4]diazocin-5(14H)-one

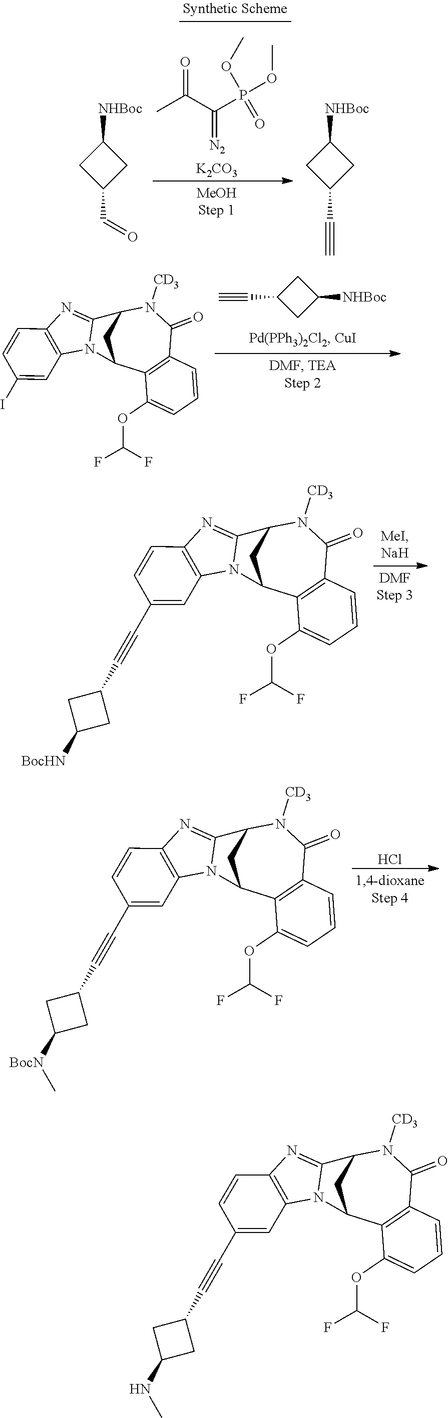

### Preparation 254A: tert-butyl ((1r,3r)-3-ethynylcyclobutyl)carbamate

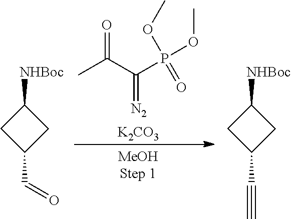

To a stirred solution of tert-butyl ((1r,3r)-3-formylcyclobutyl)carbamate (200 mg, 1.004 mmol) and K₂CO₃ (277 mg, 2.008 mmol) in MeOH (1 mL) was added dimethyl (1-diazo-2-oxopropyl)phosphonate (289 mg, 1.506 mmol) dropwise at 0° C. The resulting mixture was stirred for 3 h at room temperature. The mixture was concentrated under reduced pressure. The residue was purified by silica gel column chromatography, eluted with PE/EA (10/1) to afford tert-butyl ((1r,3r)-3-ethynylcyclobutyl)carbamate (88 mg, 44%) as a white solid. MS ESI calculated for C₁₁H₁₇NO₂ [M+H]⁺, 196.13 found N/A. ¹H NMR (400 MHz, Chloroform-d S 4.11-3.92 (m, 1H), 2.68-2.56 (m, 1H), 2.51-2.40 (m, 1H), 2.28-2.07 (m, 2H), 2.02-1.85 (m, 2H), 1.43 (s, 9H).

### Preparation 254B: tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)arbamate

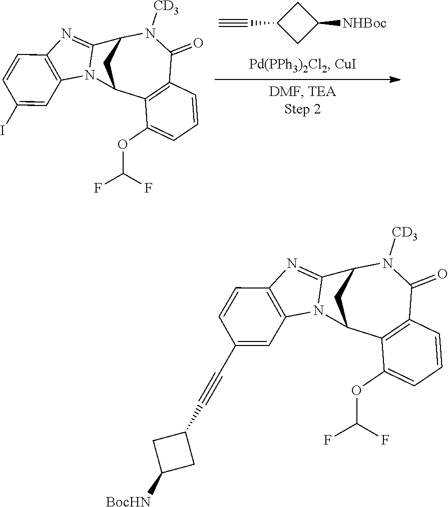

To a stirred mixture of (7R,14R)-1-(difluoromethoxy)-1-iodo-6-(methyl-d3)-6,7-dihydro-7,14-methanobenzo[f]benzo[4,5]imidazo[1,2-a][1,4]diazocin-5(14H)-one (168 mg, 0.347 mmol) and tert-butyl ((1r,3r)-3-ethynylcyclobutyl)carbamate (88 mg, 0.451 mmol) in DMF (0.5 mL) and TEA (0.5 mL) were added CuI (6 mg, 0.035 mmol) and Pd(PPh₃)₂Cl₂ (24 mg, 0.035 mmol). The resulting mixture was stirred for 2 h at 80° C. under nitrogen atmosphere. The mixture was diluted with water (10 mL), and extracted with CH₂Cl₂ (3×10 mL). The combined organic layers were washed with brine (10 mL), dried over anhydrous Na₂SO₄, filtered and concentrated under reduced pressure. The residue was purified by reversed-phase flash chromatography with the following conditions: column, C18 silica gel; mobile phase, CH₃CN in Water (10 mmol/L NH₄HCO₃), 10% to 49% gradient in 10 min; detector, 254 nm to afford tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)carbamate (68 mg, 35%) as a white solid. MS ESI calculated for C₃₀H₂₇D₃F₂N₄O₄[M+H]⁺, 552.24. found 552.20. ¹H NMR (400 MHz, Chloroform-d) δ 8.52-8.45 (m, 1H), 7.67-7.55 (m, 1H), 7.53-7.45 (m, 1H), 7.44-7.38 (m, 1H), 7.37-7.31 (m, 1H), 7.02-6.63 (m, 1H), 6.24-6.17 (m, 1H), 4.92 (s, 1H), 4.81-4.65 (m, 1H), 4.15-3.98 (m, 1H), 3.48-3.37 (m, 1H), 2.90-2.84 (m, 1H), 2.84-2.73 (m, 2H), 2.71-2.52 (m, 1H), 2.34-2.15 (m, 1H), 2.11-1.99 (m, 2H), 1.47-1.44 (m, 9H).

### Preparation 254C: tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)(methyl)carbamate

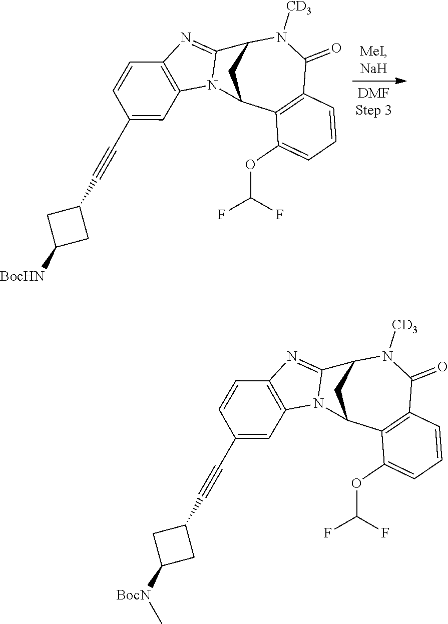 [SMILES: [2H]C([2H])([2H])N1C(=O)c2cccc(OC(F)F)c2[C@H]2C[C@@H]1c1nc3ccc(C#C[C@H]4C[C@H](NC(=O)OC(C)(C)C)C4)cc3n12 ; [2H]C([2H])([2H])N1C(=O)c2cccc(OC(F)F)c2[C@H]2C[C@@H]1c1nc3ccc(C#C[C@H]4C[C@H](N(C)C(=O)OC(C)(C)C)C4)cc3n12]

To a stirred solution of tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)carbamate (65 mg, 0.118 mmol) in DMF (0.5 mL) was added NaH (7 mg, 0.177 mmol) at 0° C. The resulting mixture was stirred for 0.5 h at 0° C. To the above mixture was added MeI (0.01 mL, 0.142 mmol) dropwise at 0° C. The resulting mixture was stirred for additional 2 h at room temperature. The reaction was quenched by the addition of water (1 mL). The mixture was extracted with EA (3×10 mL). The combined organic layers were washed with brine (10 mL), dried over anhydrous Na₂SO₄, filtered and concentrated under reduced pressure. The residue was purified by reversed-phase flash chromatography with the following conditions: column, C18 silica gel; mobile phase, CH₃CN in Water (10 mmol/L NH₄HCO₃), 10% to 50% gradient in 10 min; detector, 254 nm to afford tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)(methyl)carbamate (54 mg, 81%) as a white solid. MS ESI calculated for C₃₁H₂₉D₃F₂N₄O₄[M+H]⁺, 566.25 found 566.30. ¹H NMR (400 MHz, Chloroform-d) δ 8.49 (dd, J=8.2, 1.3 Hz, 1H), 7.68-7.60 (m, 1H), 7.55-7.48 (m, 1H), 7.43 (t, J=8.2 Hz, 1H), 7.39-7.26 (m, 2H), 7.05-6.63 (m, 1H), 6.22 (d, J=7.3 Hz, 1H), 5.01 (d, J=6.9 Hz, 1H), 3.52-3.40 (m, 1H), 2.96-2.76 (m, 5H), 2.64-2.46 (m, 2H), 2.42-2.21 (m, 3H), 1.47 (s, 9H).

### Example 254: (7R,14R)-1-(difluoromethoxy)-6-(methyl-d3)-11-(((1r,3R)-3-(methylamino)cyclobutyl)ethynyl)-6,7-dihydro-7,14-methanobenzo[f]benzo[4,5]imidazo[1,2-a][1,4]diazocin-5(14H)-one

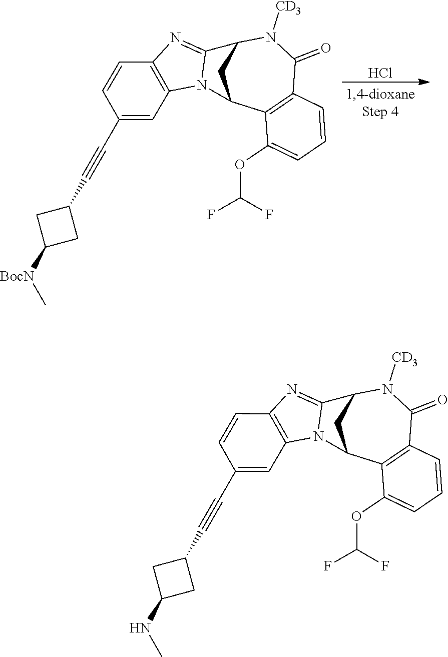 [SMILES: [2H]C([2H])([2H])N1C(=O)c2cccc(OC(F)F)c2[C@H]2C[C@@H]1c1nc3ccc(C#C[C@H]4C[C@H](N(C)C(=O)OC(C)(C)C)C4)cc3n12 ; Cl ; [2H]C([2H])([2H])N1C(=O)c2cccc(OC(F)F)c2[C@H]2C[C@@H]1c1nc3ccc(C#C[C@H]4C[C@H](NC)C4)cc3n12]

To a stirred solution of tert-butyl ((1R,3r)-3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)cyclobutyl)(methyl)carbamate (45 mg, 0.080 mmol) in 1,4-dioxane (1 mL) was added HCl (1 mL, 4 M in 1,4-dioxane) at room temperature. The resulting mixture was stirred for 1 h at room temperature. The mixture was concentrated under reduced pressure. The residue was purified by reversed-phase flash chromatography with the following conditions: column, C18 silica gel; mobile phase, CH₃CN in water (10 mmol/L NH₄HCO₃), 10% to 41% gradient in 10 min; detector, 254 nm to afford (7R,14R)-1-(difluoromethoxy)-6-(methyl-d3)-11-(((1r,3R)-3-(methylamino)cyclobutyl)ethynyl)-6,7-dihydro-7,14-methanobenzo[f]benzo[4,5]imidazo[1,2-a][1,4]diazocin-5(14H)-one (11 mg, 31%) as a white solid. MS ESI calculated for C₂₆H₂₁D₃F₂N₄O₂ [M+H]⁺, 466.21. found 466.15. ¹H NMR (400 MHz, Chloroform-d) δ 8.47 (d, J=8.1 Hz, 1H), 7.59 (d, J=8.5 Hz, 1H), 7.47 (s, 1H), 7.45-7.36 (m, 1H), 7.34 (d, J=8.0 Hz, 1H), 7.27-7.22 (m, 1H), 6.86 (t, J=73.4 Hz, 1H), 6.16 (d, J=7.3 Hz, 1H), 4.92 (d, J=7.2 Hz, 1H), 3.85-3.36 (m, 2H), 3.27-3.05 (m, 1H), 2.97-2.61 (m, 4H), 2.53-2.23 (m, 3H), 2.09-1.94 (m, 2H). ¹⁹F NMR (377 MHz, Chloroform-d) δ −79.82, −80.27, −81.27, −81.71.

### Example 255: N-(5-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)pyridin-2-yl)acetamide

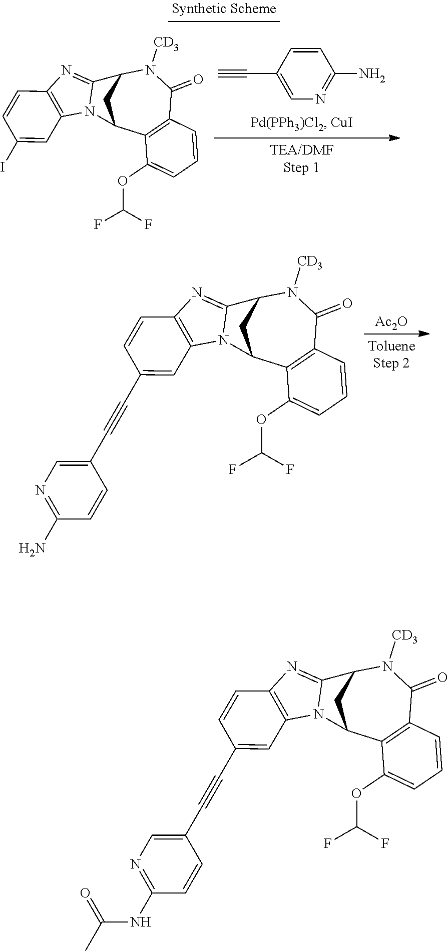

### Preparation 255A: (7R,14R)-11-((6-aminopyridin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one

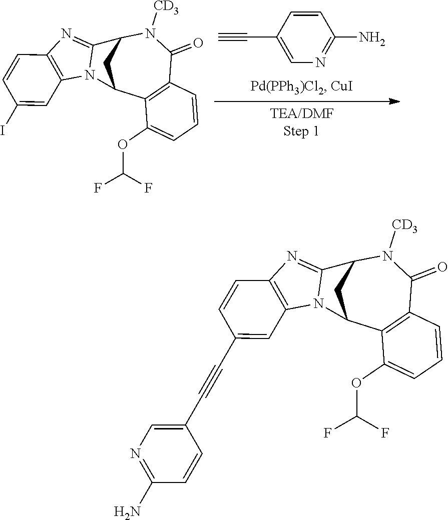

A mixture of (7R,14R)-1-(difluoromethoxy)-11-iodo-6-(methyl-d₃)-6,7-dihydro-7,14-methanobenzo[f]benzo[4,5]imidazo[1,2-a][1,4]diazocin-5(14H)-one (100 mg, 0.210 mmol), 5-ethynylpyridin-2-amine (29 mg, 0.250 mmol), Pd(PPh₃)2C₂ (14 mg, 0.020 mmol), CuI (4 mg, 0.020 mmol) in TEA (1 mL) and DMF (1 mL) was stirred for 2 h at 80° C. under nitrogen atmosphere. The resulting mixture was concentrated under reduced pressure and purified by silica gel column chromatography, eluted with DCM/MeOH (10/1) to afford (7R,14R)-11-((6-aminopyridin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one as (92 mg, 94%) a yellow solid. MS ESI calculated for C₂₆H₁₆D₃F₂N₅O₂ [M+H]+ 475.17 found 474.25. ¹H NMR (400 MHz, Chloroform-d) δ 8.54-8.43 (m, 1H), 8.23 (s, 1H), 7.72-7.51 (m, 3H), 7.47-7.31 (m, 3H), 6.85 (t, J=72.2 Hz, 1H), 6.58 (d, J=8.6 Hz, 1H), 6.23 (d, J=7.2 Hz, 1H), 4.95 (d, J=7.1 Hz, 1H), 3.54-3.42 (m, 1H), 2.87 (d, J=13.5 Hz, 1H). ¹⁹F NMR (377 MHz, Chloroform-d) δ −79.89, −80.34, −81.12, −81.56.

### Example 255: N-(5-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)pyridin-2-yl)acetamide

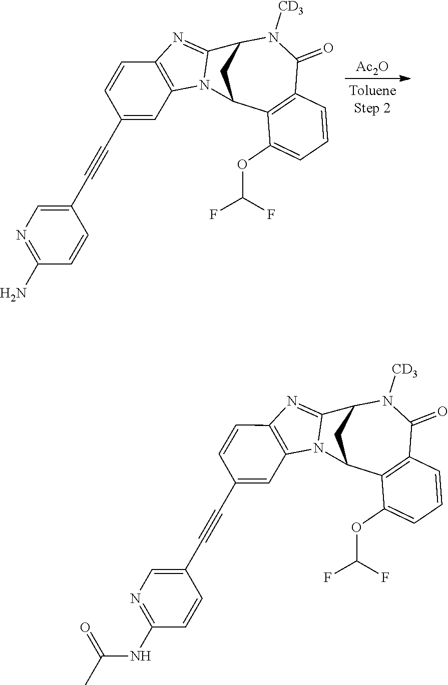 [SMILES: "Ac2O" ; [2H]C([2H])([2H])N1C(=O)c2cccc(OC(F)F)c2[C@H]2C[C@@H]1c1nc3ccc(C#Cc4ccc(N)nc4)cc3n12 ; [2H]C([2H])([2H])N1C(=O)c2cccc(OC(F)F)c2[C@H]2C[C@@H]1c1nc3ccc(C#Cc4ccc(NC(C)=O)nc4)cc3n12]

A solution of (7R,14R)-11-((6-aminopyridin-3-yl)ethynyl)-1-(difluoromethoxy)-6-(methyl-d₃)-6,7-dihydro-7,14-methanobenzo[f]benzo[4,5]imidazo[1,2-a][1,4]diazocin-5(14H)-one (60 mg, 0.130 mmol) in acetic anhydride (1.5 mL) and toluene (0.2 mL) was stirred for 2 h at 100° C. The resulting mixture was concentrated under reduced pressure and purified by reversed-phase flash chromatography with the following conditions: column, C18 silica gel; mobile phase, CH₃CN in Water (10 mmol/L NH₄HCO₃), 35% to 45% gradient in 10 min; detector, 254 nm. This resulted in N-(5-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)pyridin-2-yl)acetamide as a white solid (33 mg, 51%). MS ESI calculated for C₂₈H₁₈D₃F₂NO₃ [M+H]⁺, 517.18 found 517.15. ¹H NMR (400 MHz, Chloroform-d) δ 8.86 (s, 1H), 8.55-8.48 (m, 1H), 8.40 (d, J=2.2 Hz, 1H), 8.32 (d, J=8.8 Hz, 1H), 7.98-7.90 (m, 1H), 7.72 (d, J=8.5 Hz, 1H), 7.65 (s, 1H), 7.45 (t, J=8.2 Hz, 2H), 7.36 (d, J=8.2 Hz, 1H), 6.86 (t, J=72.7 Hz, 1H), 6.27 (d, J=7.2 Hz, 1H), 5.04 (d, J=7.1 Hz, 1H), 3.57-3.43 (m, 1H), 2.90 (d, J=13.6 Hz, 1H), 2.27 (s, 3H). ¹⁹F NMR (377 MHz, Chloroform-d) δ −80.05, −80.49, −80.99, −81.43.

### Example 256: (7R,14R)-1-(difluoromethoxy)-11-((2,2-dimethyl-1-(oxetan-3-yl)azetidin-3-yl)ethynyl)-6-(methyl-d₃)-6,7-dihydro-7,14-methanobenzo[f]benzo[4,5]imidazo[1,2-a][1,4]diazocin-5(14H)-one

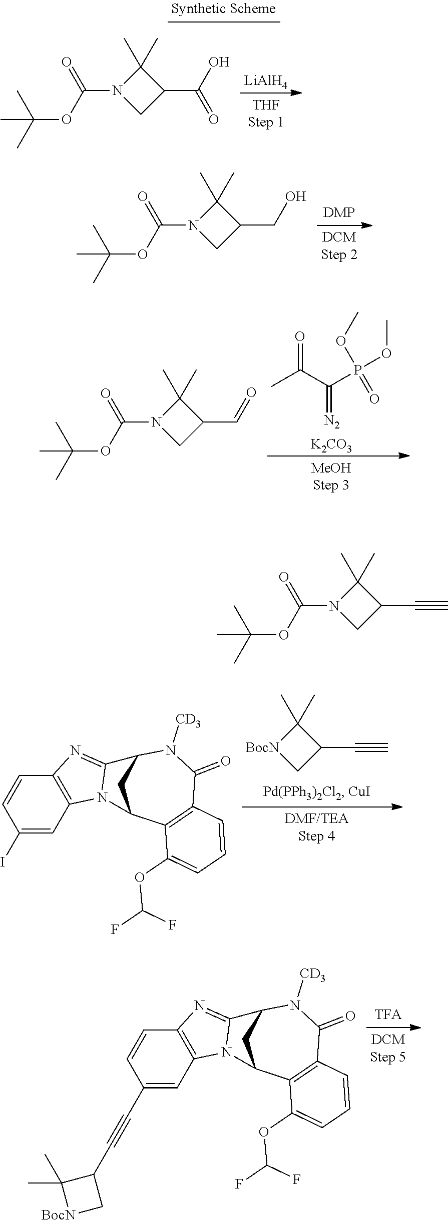

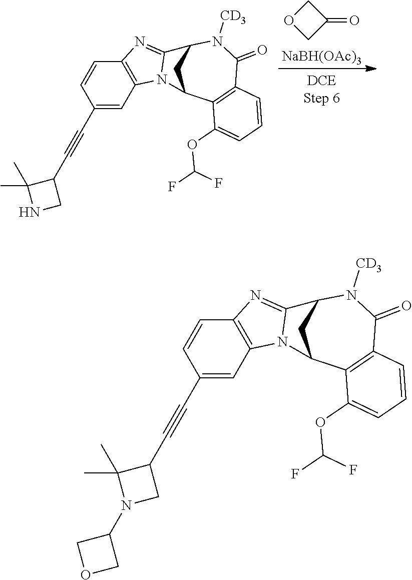

### Preparation 256A: tert-butyl 3-(hydroxymethyl)-2,2-dimethylazetidine-1-carboxylate

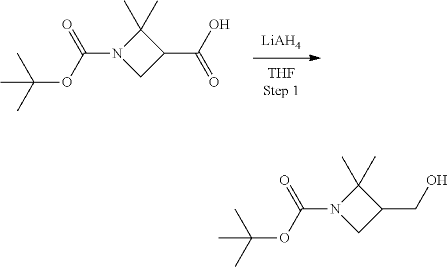 [SMILES: CC(C)(C)OC(=O)N1CC(C(=O)O)C1(C)C ; "LiAH4" ; CC(C)(C)OC(=O)N1CC(CO)C1(C)C]

To a stirred solution of LiAlH₄ (2 mL, 2.00 mmol/L) in THF (4 mL) was added 1-(tert-butoxycarbonyl)-2,2-dimethylazetidine-3-carboxylic acid (570 mg, 2.486 mmol) at 0° C. The resulting mixture was stirred for 1 h at 0° C. under nitrogen atmosphere. The reaction was quenched by the addition of water (20 mL) at 0° C. The resulting mixture was extracted with EtOAc (3×15 mL). The combined organic layers were washed with brine (3×15 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in Water (0.1% FA), 20% to 50% gradient in 30 min; detector, 254 nm to afford tert-butyl 3-(hydroxymethyl)-2,2-dimethylazetidine-1-carboxylate (505 mg, 94%) as a colorless oil. MS ESI calculated for C₁₁H₂₁NO₃ [M+H]⁺, 216.15 found 160.00. ¹H NMR (400 MHz, Chloroform-d) δ 3.92-3.81 (m, 2H), 3.78-3.70 (m, 1H), 3.47-3.37 (m, 1H), 2.48-2.31 (m, 1H), 1.54-1.34 (m, 15H).

### Preparation 256B: tert-butyl 3-formyl-2,2-dimethylazetidine-1-carboxylate

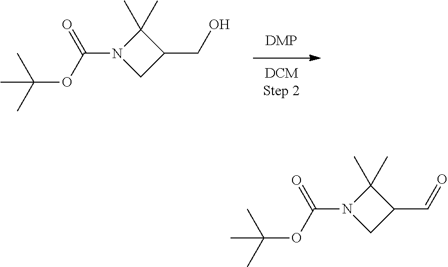

To a stirred solution of tert-butyl 3-(hydroxymethyl)-2,2-dimethylazetidine-1-carboxylate (599 mg, 2.780 mmol) in DCM (15 mL) was added DMP (1.20 g, 2.780 mmol) in portions at 0° C. The resulting mixture was stirred for 3 h at room temperature. The reaction was quenched by the addition of sat. sodium hyposulfite (30 mL) and sat. NaHCO₃ (30 mL) at 0° C. The resulting mixture was extracted with CH₂Cl₂ (3×20 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EA (1:1) to afford tert-butyl 3-formyl-2,2-dimethylazetidine-1-carboxylate (500 mg, 84%) as a colorless oil. MS ESI calculated for C₁₁H₁₉NO₃ [M+H]⁺, 214.14 found N/A. ¹H NMR (400 MHz, Chloroform-d) δ 9.86 (d, J=2.5 Hz, 1H), 4.20-4.11 (m, 1H), 3.85 (t, J=8.7 Hz, 1H), 3.10-2.97 (m, 1H), 1.60 (s, 3H), 1.45 (s, 9H), 1.42 (s, 3H).

### Preparation 256C: tert-butyl 3-ethynyl-2,2-dimethylazetidine-1-carboxylate

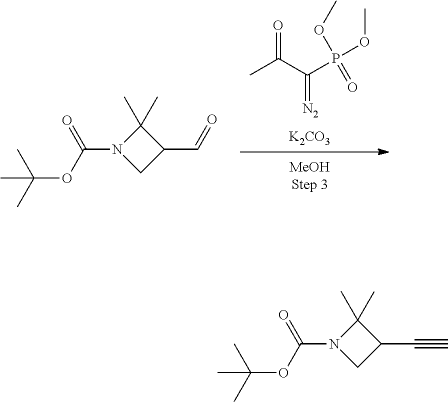

To a stirred mixture of tert-butyl 3-formyl-2,2-dimethylazetidine-1-carboxylate (250 mg, 1.170 mmol) and K₂CO₃ (648 mg, 4.690 mmol) in MeOH (5 mL) was added dimethyl (1-diazo-2-oxopropyl)phosphonate (270 mg, 1.410 mmol) at 0° C. The resulting mixture was stirred for 3 h at room temperature. The reaction concentrated under reduced pressure. The residue was purified by silica gel column chromatography, eluted with PE/EA (1:1) to afford tert-butyl 3-ethynyl-2,2-dimethylazetidine-1-carboxylate (148 mg, 60%) as a colorless oil. MS ESI calculated for C₁₂H₁₉NO₂ [M+H]⁺, 210.14 found N/A. ¹H NMR (400 MHz, Chloroform-d) δ 3.98 (t, J=8.6 Hz, 1H), 3.75-3.65 (m, 1H), 3.08-2.97 (m, 1H), 2.29 (s, 1H), 1.53-1.39 (m, 15H).

### Preparation 256D: tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)-2,2-dimethylazetidine-1-carboxylate

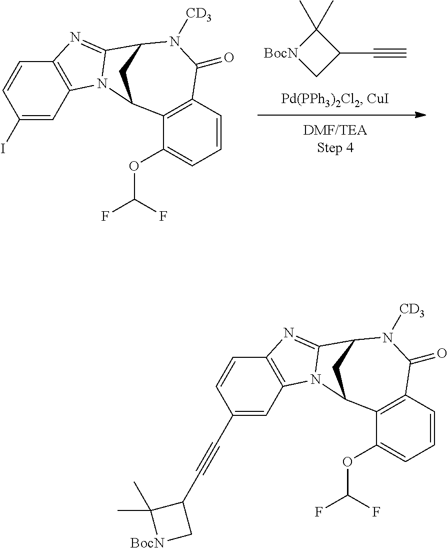 [SMILES: [2H]C([2H])([2H])N1C(=O)c2cccc(OC(F)F)c2[C@H]2C[C@@H]1c1nc3ccc(I)cc3n12 ; C#CC1CN(C(=O)OC(C)(C)C)C1(C)C ; [2H]C([2H])([2H])N1C(=O)c2cccc(OC(F)F)c2[C@H]2C[C@@H]1c1nc3ccc(C#CC4CN(C(=O)OC(C)(C)C)C4(C)C)cc3n12]

To a stirred mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (100 mg, 0.207 mmol) and tert-butyl 3-ethynyl-2,2-dimethylazetidine-1-carboxylate (56 mg, 0.269 mmol) in DMF (2 mL) and TEA (2 mL) were added CuI (4 mg, 0.021 mmol) and Pd(PPh₃)₂Cl₂ (14 mg, 0.021 mmol) at room temperature. The resulting mixture was stirred for 1 h at 80° C. under nitrogen atmosphere. The resulting mixture was diluted with water (10 mL) and extracted with CH₂Cl₂/MeOH (10:1) (3×10 mL). The combined organic layers were washed with brine (5×50 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 3-(((7R,14R)-1-(difluoromethoxy)-6-(methyl-d₃)-5-oxo-5,6,7,14-tetrahydro-7,14-methanobenzo[f]benzo[4,5]imidazo[1,2-a][1,4]diazocin-11-yl)ethynyl)-2,2-dimethylazetidine-1-carboxylate (90 mg, 77%) as a yellow oil. MS ESI calculated for C₃₁H₂₉D₃F₂N₄O₄[M+H]⁺, 566.26. found 566.30.

### Preparation 256E: (7R,14R)-1-(difluoromethoxy)-11-((2,2-dimethylazetidin-3-yl)ethynyl)-6-(methyl-d₃)-6,7-dihydro-7,14-methanobenzo[f]benzo[4,5]imidazo[1,2-a][1,4]diazocin-5(14H)-one

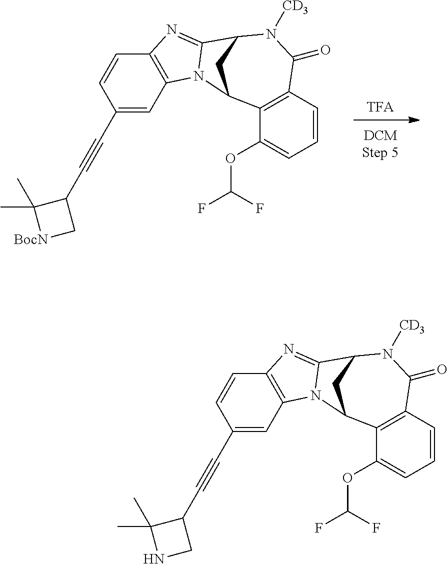 [SMILES: [2H]C([2H])([2H])N1C(=O)c2cccc(OC(F)F)c2[C@H]2C[C@@H]1c1nc3ccc(C#CC4CN(C(=O)OC(C)(C)C)C4(C)C)cc3n12 ; "TFA" ; [2H]C([2H])([2H])N1C(=O)c2cccc(OC(F)F)c2[C@H]2C[C@@H]1c1nc3ccc(C#CC4CNC4(C)C)cc3n12]

To a stirred solution of tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-2,2-dimethylazetidine-1-carboxylate (85 mg, 0.150 mmol) in DCM (0.5 mL) was added TFA (0.1 mL) dropwise at room temperature. The resulting mixture was stirred for 1 h at room temperature. The resulting mixture was concentrated under reduced pressure to afford (7R,14R)-1-(difluoromethoxy)-11-((2,2-dimethylazetidin-3-yl)ethynyl)-6-(methyl-d₃)-6,7-dihydro-7,14-methanobenzo[f]benzo[4,5]imidazo[1,2-a][1,4]diazocin-5(14H)-one (60 mg, 86%) as a yellow solid. The crude product was used in the next step directly without further purification. MS ESI calculated for C₂₆H₂₁D₃F₂N₄O₂ [M+H]⁺, 466.21. found 466.20.

### Example 256: (7R,14R)-1-(difluoromethoxy)-11-((2,2-dimethyl-1-(oxetan-3-yl)azetidin-3-yl)ethynyl)-6-(methyl-d₃)-6,7-dihydro-7,14-methanobenzo[f]benzo[4,5]imidazo[1,2-a][1,4]diazocin-5(14H)-one

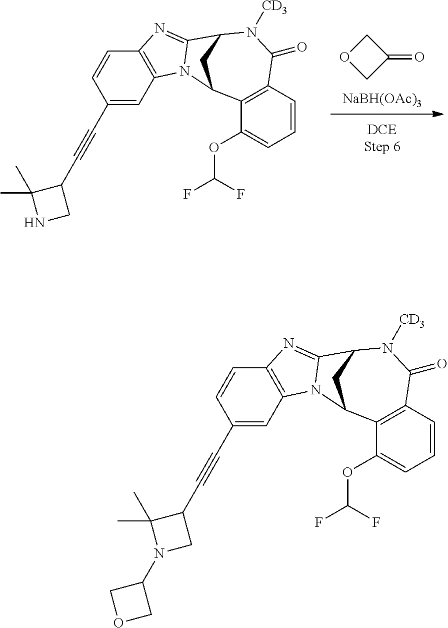

To a stirred mixture of (7R,14R)-1-(difluoromethoxy)-11-((2,2-dimethylazetidin-3-yl)ethynyl)-6-(methyl-d3)-6,7-dihydro-7,14-methanobenzo[f]benzo[4,5]imidazo[1,2-a][1,4]diazocin-5(14H)-one (50 mg, 0.107 mmol) and 3-oxetanone (39 mg, 0.535 mmol) in DCE (2 mL) was added sodium triacetoxyborohydride (36 mg, 0.171 mmol) at room temperature. The resulting mixture was stirred for 2 h at room temperature. The mixture basified to pH 8 with saturated NaHCO₃(aq.). The resulting mixture was extracted with CH₂Cl₂/MeOH (10:1) (3×10 mL). The combined organic layers were washed with brine (3×10 mL), dried over anhydrous Na₂SO₄. After filtration, the filtrate was concentrated under reduced pressure and purified by reverse phase flash with the following conditions (Column: XBridge Prep Phenyl OBD Column 19*250 mm, 5m; Mobile Phase A: Water (10 mmol/L NH₄HCO₃+0.1% NH₃·H₂O), Mobile Phase B: ACN; Flow rate: 60 mL/min; Gradient: 24% B to 39% B in 10 min; Wave Length: 254 nm; RT1(min): 11.589) to afford (7R,14R)-1-(difluoromethoxy)-11-((2,2-dimethyl-1-(oxetan-3-yl)azetidin-3-yl)ethynyl)-6-(methyl-d₃)-6,7-dihydro-7,14-methanobenzo[f]benzo[4,5]imidazo[1,2-a][1,4]diazocin-5(14H)-one (16 mg, 29%) as a white solid. MS ESI calculated for C₂₉H₂₅D₃F₂N₄O₃[M+H]⁺, 522.23 found 522.15. ¹H NMR (400 MHz, Chloroform-d) δ 8.54-8.43 (m, 1H), 7.61 (d, J=8.4 Hz, 1H), 7.52 (d, J=1.4 Hz, 1H), 7.42 (t, J=8.2 Hz, 1H), 7.35-7.30 (m, 1H), 7.29-7.25 (m, 1H), 6.84 (t, J=72.9 Hz, 1H), 6.20 (d, J=7.2 Hz, 1H), 4.92 (d, J=7.1 Hz, 1H), 4.74 (t, J=6.3 Hz, 1H), 4.71-4.54 (m, 3H), 4.02 (p, J=6.7 Hz, 1H), 3.77-3.68 (m, 1H), 3.50-3.38 (m, 1H), 3.36-3.23 (m, 2H), 2.85 (d, J=13.5 Hz, 1H), 1.31 (s, 3H), 1.24 (s, 3H). ¹⁹F NMR (377 MHz, Chloroform-d) δ −80.06, −80.07, −80.51, −80.52, −80.91, −80.92, −81.36, −81.37.

### Example 257 and 259: (7R,14R)-1-(difluoromethoxy)-6-(methyl-d3)-11-(((2S or 2R)-2-methyl-1-(oxetan-3-yl)azetidin-3-yl)ethynyl)-6,7-dihydro-7,14-methanobenzo[f]benzo[4,5]imidazo[1,2-a][1,4]diazocin-5(14H)-one and (7R,14R)-1-(difluoromethoxy)-6-(methyl-d3)-11-(((2R or 2S)-2-methyl-1-(oxetan-3-yl)azetidin-3-yl)ethynyl)-6,7-dihydro-7,14-methanobenzo[f]benzo[4,5]imidazo[1,2-a][1,4]diazocin-5(14H)-one

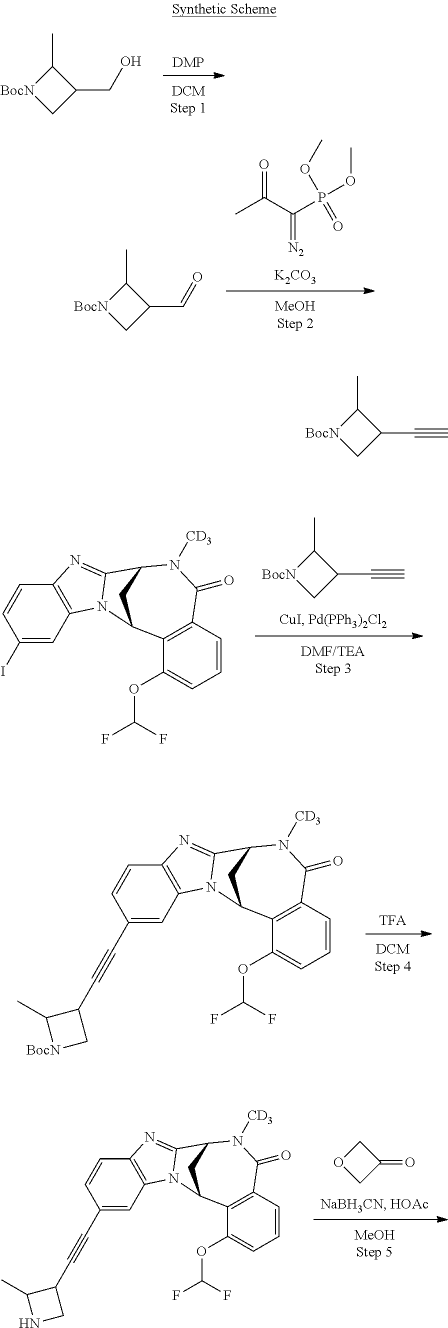

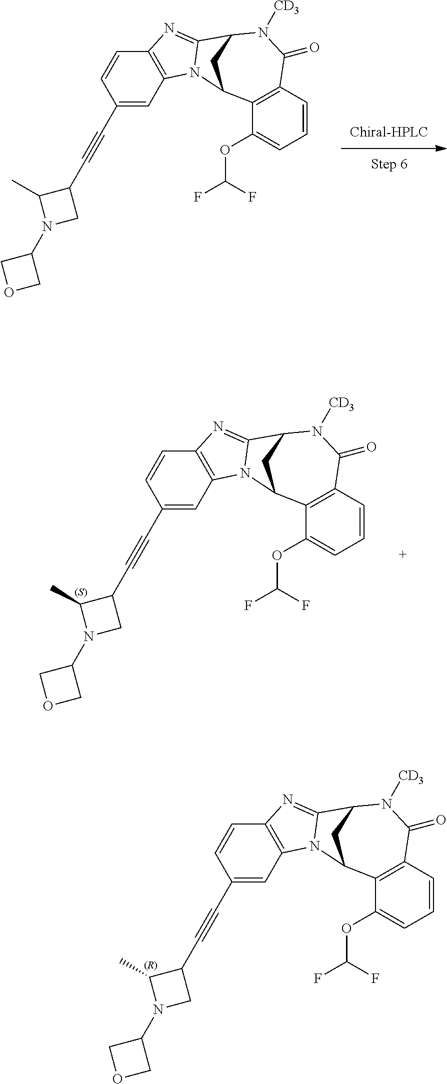

### Preparation 257A: tert-butyl 3-formyl-2-methylazetidine-1-carboxylate

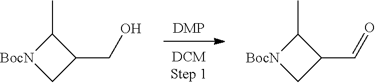

A mixture of tert-butyl 3-(hydroxymethyl)-2-methylazetidine-1-carboxylate (700 mg, 3.478 mmol) and DMP (1.77 g, 4.174 mmol) in DCM (10 mL) was stirred for 2 h at room temperature. The reaction was quenched by the addition of sat. Na₂S₂O₃ (aq.) (10 mL) and sat. NaHCO₃ (aq.) (10 mL) at room temperature. The resulting mixture was extracted with EtOAc (3×20 mL). The combined organic layers were washed with brine (3×20 mL), dried over anhydrous MgSO₄. After filtration, the filtrate was concentrated under reduced pressure. The residue was purified by silica gel column chromatography, eluted with PE/EA (1:1) to afford tert-butyl 3-formyl-2-methylazetidine-1-carboxylate (550 mg, 79%) as an off-white solid. MS ESI calculated for C₁₀H₁₇NO₃ [M+H]⁺, 200.12 found N/A.

### Preparation 257B: tert-butyl 3-ethynyl-2-methylazetidine-1-carboxylate

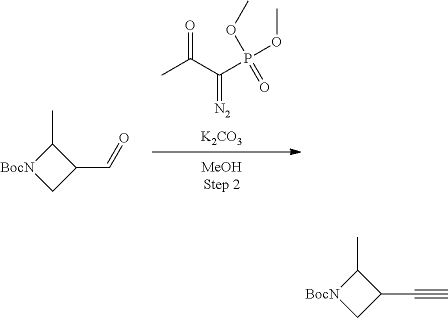

To a solution of tert-butyl 3-formyl-2-methylazetidine-1-carboxylate (500 mg, 2.509 mmol) in MeOH (10 mL) was added K₂CO₃ (1.37 g, 10.036 mmol) and seyferth-gilbert homologation (578 mg, 3.011 mmol) and the mixture stirred for 3 h. The resulting mixture was diluted with water (10 mL). The resulting mixture was extracted with EtOAc (3×20 mL). The combined organic layers were washed with brine (2×10 mL), dried over anhydrous Na₂SO₄.

After filtration, the filtrate was concentrated under reduced pressure. The residue was purified by silica gel column chromatography, eluted with PE/EA (2:1) to afford tert-butyl 3-ethynyl-2-methylazetidine-1-carboxylate (150 mg, 30%) as an off-white solid. MS ESI calculated for C₁₁H₁₇NO₂ [M+H]⁺, 196.13 found N/A. ¹H NMR (400 MHz, Chloroform-d) δ 4.33-4.17 (m, 1H), 4.01 (t, J=8.4 Hz, 1H), 3.79 (t, J=7.6 Hz, 1H), 2.84-2.76 (m, 1H), 2.30 (d, J=2.4 Hz, 1H), 1.44 (s, 9H), 1.43-1.41 (m, 3H).

### Preparation 257C: tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-2-methylazetidine-1-carboxylate

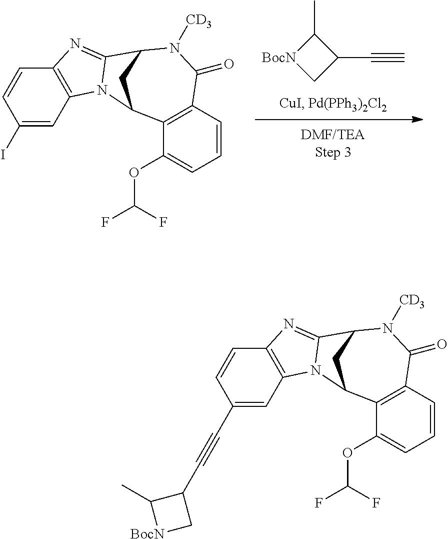 [SMILES: [2H]C([2H])([2H])N1C(=O)c2cccc(OC(F)F)c2[C@H]2C[C@@H]1c1nc3ccc(I)cc3n12 ; C#CC1CN(C(=O)OC(C)(C)C)C1C ; [2H]C([2H])([2H])N1C(=O)c2cccc(OC(F)F)c2[C@H]2C[C@@H]1c1nc3ccc(C#CC4CN(C(=O)OC(C)(C)C)C4C)cc3n12]

To a stirred mixture of (7R,14R)-1-(difluoromethoxy)-11-iodo-6-(methyl-d3)-6,7-dihydro-7,14-methanobenzo [f]benzo [4,5]imidazo [1,2-a][1,4]diazocin-5(14H)-one (280 mg, 0.578 mmol), tert-butyl 3-ethynyl-2-methylazetidine-1-carboxylate (146 mg, 0.751 mmol) and CuI (11 mg, 0.058 mmol) in TEA (2 mL) and DMF (2 mL) were added Pd(PPh₃)₂Cl₂ (40 mg, 0.058 mmol) at room temperature. The resulting mixture was stirred for 1 h at 80° C. under nitrogen atmosphere. The resulting mixture was diluted with water (10 mL). The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (9:1) to afford tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-2-methylazetidine-1-carboxylate (240 mg, 75%) as an off-white solid. MS ESI calculated for C₃₀H₂₇D₃F₂N₄O₄[M+H]⁺, 552.24. found 552.30. ¹⁹F NMR (377 MHz, Chloroform-d) δ −81.73, −82.18, −82.25, −82.70.

### Preparation 257D: (7R,14R)-1-(difluoromethoxy)-6-(methyl-d3)-11-((2-methylazetidin-3-yl)ethynyl)-6,7-dihydro-7,14-methanobenzo[f]benzo[4,5]imidazo[1,2-a][1,4]diazocin-5(14H)-one

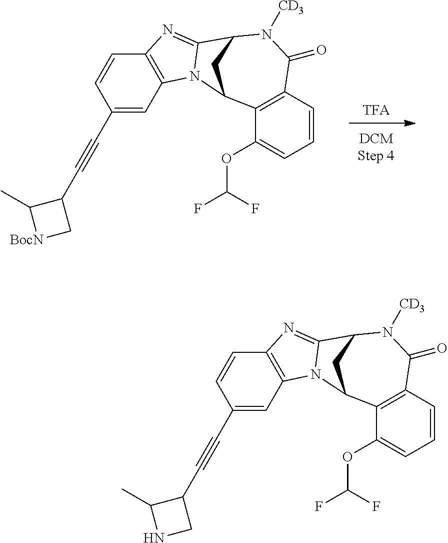 [SMILES: "TFA" ; [2H]C([2H])([2H])N1C(=O)c2cccc(OC(F)F)c2[C@H]2C[C@@H]1c1nc3ccc(C#CC4CN(C(=O)OC(C)(C)C)C4C)cc3n12 ; [2H]C([2H])([2H])N1C(=O)c2cccc(OC(F)F)c2[C@H]2C[C@@H]1c1nc3ccc(C#CC4CNC4C)cc3n12]

Into a 40 mL vial were added tert-butyl 3-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-2-methylazetidine-1-carboxylate (240 mg, 0.435 mmol) in DCM (5 mL) was added trifluoroacetaldehyde (1 mL) at room temperature. The resulting mixture was stirred for 1 h at room temperature. The resulting mixture was diluted with water (10 mL). The mixture was neutralized to pH 7 with saturated NaHCO₃(aq.). The resulting mixture was extracted with EtOAc (3×10 mL). The combined organic layers were washed with brine (2×5 mL), dried over anhydrous Na₂SO₄. After filtration, the filtrate was concentrated under reduced pressure. The residue was purified by silica gel column chromatography, eluted with CH₂Cl₂/MeOH (4:1) to afford (7R,14R)-1-(difluoromethoxy)-6-(methyl-d3)-11-((2-methylazetidin-3-yl)ethynyl)-6,7-dihydro-7,14-methanobenzo[f]benzo[4,5]imidazo[1,2-a][1,4]diazocin-5(14H)-one (150 mg, 76%) as an off-white solid. MS ESI calculated for C₂₅H₁₉D₃F₂N₄O₂ [M+H]⁺, 452.19. found 452.15. ¹H NMR (400 MHz, Chloroform-d) δ 8.48 (d, J=8.2 Hz, 1H), 7.61 (d, J=8.5 Hz, 1H), 7.56-7.24 (m, 4H), 6.84 (t, J=72.7 Hz, 1H), 6.19 (d, J=7.3 Hz, 1H), 4.92 (d, J=7.3 Hz, 1H), 4.24-4.04 (m, 1H), 3.83-3.59 (m, 2H), 3.55-3.38 (m, 1H), 3.29-3.13 (m, 1H), 2.84 (d, J=13.6 Hz, 1H), 1.35 (d, J=6.2 Hz, 3H). ¹⁹F NMR (377 MHz, Chloroform-d) δ −79.92, −80.31, −81.07, −81.6.

### Preparation 257E: (7R,14R)-1-(difluoromethoxy)-6-(methyl-d3)-11-((2-methyl-1-(oxetan-3-yl) azetidin-3-yl) ethynyl)-6,7-dihydro-7,14-methanobenzo [f]benzo [4,5]imidazo [1,2-a][1,4]diazocin-5(14H)-one

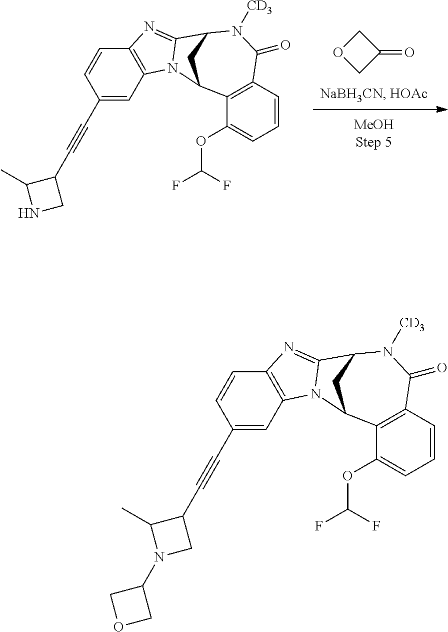

To a stirred mixture of (7R,14R)-1-(difluoromethoxy)-6-(methyl-d3)-11-((2-methylazetidin-3-yl)ethynyl)-6,7-dihydro-7,14-methanobenzo[f]benzo[4,5]imidazo[1,2-a][1,4]diazocin-5(14H)-one (34 mg, 0.465 mmol), 3-oxetanone (34 mg, 0.465 mmol) and AcOH (56 mg, 0.930 mmol) in MeOH (3 mL) was added NaBH₃CN (30 mg, 0.465 mmol) at room temperature. The resulting mixture was stirred for 12 h at room temperature under nitrogen atmosphere. The reaction was quenched by the addition of water (5 mL) at room temperature. The resulting mixture was extracted with EtOAc (3×10 mL). The combined organic layers were washed with brine (3×5 mL), dried over anhydrous Na₂SO₄. After filtration, the filtrate was concentrated under reduced pressure. The residue was purified by reversed-phase flash chromatography with the following conditions: column, C18 silica gel; mobile phase, CH₃CN in water (10 mmol/L NH₄HCO₃), 30% to 50% gradient in 20 min; detector, 254 nm to afford (7R,14R)-1-(difluoromethoxy)-6-(methyl-d3)-11-((2-methyl-1-(oxetan-3-yl) azetidin-3-yl) ethynyl)-6,7-dihydro-7,14-methanobenzo [f]benzo [4,5]imidazo [1,2-a][1,4]diazocin-5(14H)-one (80 mg, 50%) as a white solid. MS ESI calculated for C₂₈H₂₃D₃F₂N₄O₃[M+H]⁺, 508.22. found 508.15.

### Example 257 and 258: (7R,14R)-1-(difluoromethoxy)-6-(methyl-d3)-11-(((2S or 2R)-2-methyl-1-(oxetan-3-yl)azetidin-3-yl)ethynyl)-6,7-dihydro-7,14-methanobenzo[f]benzo[4,5]imidazo[1,2-a][1,4]diazocin-5(14H)-one and (7R,14R)-1-(difluoromethoxy)-6-(methyl-d3)-11-(((2R or 2S)-2-methyl-1-(oxetan-3-yl)azetidin-3-yl)ethynyl)-6,7-dihydro-7,14-methanobenzo[f]benzo[4,5]imidazo[1,2-a][1,4]diazocin-5(14H)-one

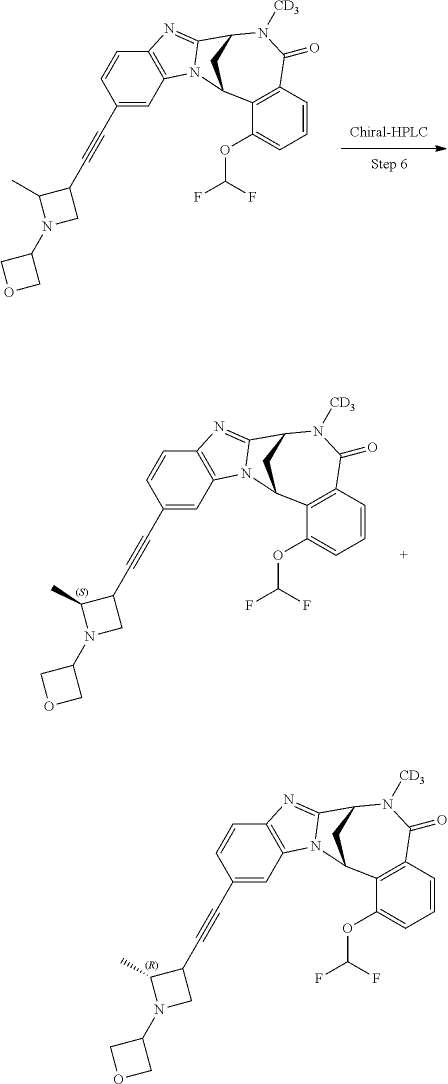

The mixture (80 mg) was purified by Prep-Chiral HPLC with the following conditions (Column: Chiral NQ(2)5u, 250*30 mm; Mobile Phase A: Hex (10 mM NH₃-MeOH), Mobile Phase B: IPA; Flow rate: 40 mL/min; Gradient: isocratic 30; Wave Length: 210/220 nm; RT1(min): 13.6; RT2(min): 17.0). The first peak to afforded (30 mg, 37%) as a white solid. MS ESI calculated for C₂₈H₂₃D₃F₂N₄O₃[M+H]⁺, 508.22. found 508.15. ¹H NMR (400 MHz, Chloroform-d) δ 8.53-8.35 (m, 1H), 7.61 (d, J=8.4 Hz, 1H), 7.51-7.48 (m, 1H), 7.42 (t, J=8.2 Hz, 1H), 7.36-7.31 (m, 1H), 7.28-7.24 (m, 1H), 6.84 (t, J=72.2 Hz, 1H), 6.20 (d, J=7.2 Hz, 1H), 4.93 (d, J=7.2 Hz, 1H), 4.79-4.72 (m, 1H), 4.71-4.66 (m, 2H), 4.61-4.55 (m, 1H), 3.89 (d, J=6.0 Hz, 1H), 3.80-3.70 (m, 1H), 3.51-3.31 (m, 2H), 3.20-3.00 (m, 2H), 2.85 (d, J=13.6 Hz, 1H), 1.38-1.32 (m, 3H). ¹⁹F NMR (376 MHz, Chloroform-d) δ −79.96, −80.40, −80.99, −81.44. The second peak to afforded (27 mg, 33%) as a white solid. MS ESI calculated for C₂₈H₂₃D₃F₂N₄O₃ [M+H]⁺, 508.22. found 508.15. ¹H NMR (400 MHz, Chloroform-d) δ 8.51-8.42 (m, 1H), 7.65-7.56 (m, 1H), 7.50-7.48 (m, 1H), 7.42 (t, J=8.2 Hz, 1H), 7.35-7.30 (m, 1H), 7.28-7.24 (m, 1H), 6.84 (t, J=72.2 Hz, 1H), 6.20 (d, J=7.2 Hz, 1H), 4.93 (d, J=7.2 Hz, 1H), 4.74 (t, J=6.8 Hz, 1H), 4.71-4.65 (m, 2H), 4.58 (t, J=6.0 Hz, 1H), 3.92-3.80 (m, 1H), 3.72 (t, J=6.8 Hz, 1H), 3.50-3.38 (m, 1H), 3.38-3.28 (m, 1H), 3.17-2.99 (m, 2H), 2.85 (d, J=13.6 Hz, 1H), 1.33 (d, J=6.2 Hz, 3H). ¹⁹F NMR (376 MHz, Chloroform-d) δ −79.94, −80.39, −81.01, −81.45.

### Example 259: N-(5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrimidin-2-yl)acetamide

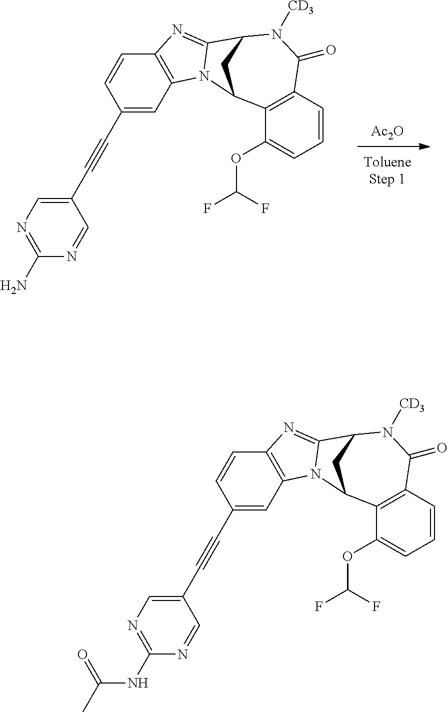 [SMILES: "Ac2O" ; [2H]C([2H])([2H])N1C(=O)c2cccc(OC(F)F)c2[C@H]2C[C@@H]1c1nc3ccc(C#Cc4cnc(N)nc4)cc3n12 ; [2H]C([2H])([2H])N1C(=O)c2cccc(OC(F)F)c2[C@H]2C[C@@H]1c1nc3ccc(C#Cc4cnc(NC(C)=O)nc4)cc3n12]

Into a 8 mL vial was added (7R,14R)-11-((2-aminopyrimidin-5-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (90 mg, 0.189 mmol) in Toluene (2 mL) and Ac₂O (0.4 mL) at room temperature. The resulting mixture was stirred for 16 h at 100° C. The resulting mixture was concentrated under reduced pressure and purified by silica gel column chromatography, eluted with CH₂Cl₂/MeOH (15:1) followed by reversed-phase flash chromatography with the following conditions: column, C18 silica gel; mobile phase, CH₃CN in Water (10 mmol/L NH₄HCO₃), 5% to 40% gradient in 25 min; detector, 254 nm to afford N-(5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)pyrimidin-2-yl)acetamide (26 mg, 27%) as a white solid. MS ESI calculated for C₂₇H₁₇D₃F₂N₆O₃ [M+H]⁺, 518.18. found 518.15. ¹H NMR (400 MHz, Chloroform-d) δ 8.70 (s, 2H), 8.55-8.45 (m, 1H), 8.13 (s, 1H), 7.79 (d, J=8.5 Hz, 1H), 7.75-7.68 (m, 1H), 7.57-7.44 (m, 2H), 7.38 (d, J=8.2 Hz, 1H), 6.88 (t, J=72.6 Hz, 1H), 6.35 (d, J=6.4 Hz, 1H), 5.32-5.18 (m, 1H), 3.61-3.51 (m, 1H), 2.96 (d, J=13.4 Hz, 1H), 2.51 (s, 3H). ¹⁹F NMR (377 MHz, Chloroform-d) δ −80.30, −80.74, −80.94, −81.38.

### Example 260: N-(5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)acetamide

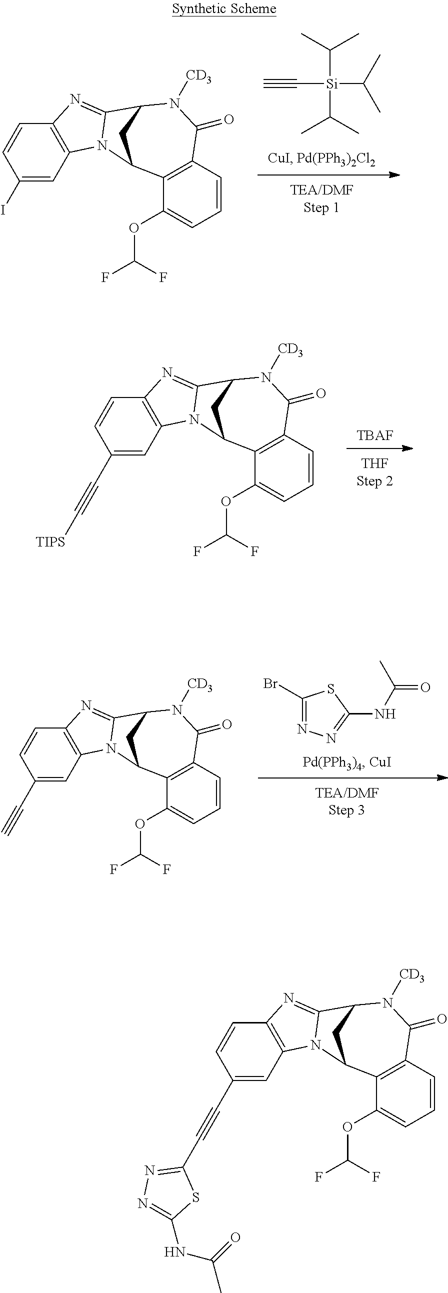

### Preparation 26A: (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one

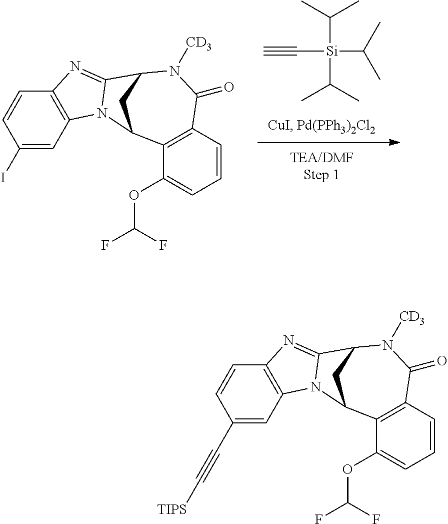

A mixture of (7R,14R)-1-(difluoromethoxy)-11-iodo-6-(methyl-d3)-6,7-dihydro-7,14-methanobenzo[f]benzo[4,5]imidazo[1,2-a][1,4]diazocin-5(14H)-one (400 mg, 0.826 mmol), ethynyltriisopropylsilane (181 mg, 0.991 mmol), CuI (15 mg, 0.083 mmol), Pd(PPh₃)Cl₂ (58 mg, 0.083 mmol) in TEA (3 mL) and DMF (3 mL) was stirred for 2 h at 80° C. under nitrogen atmosphere. The reaction was quenched by the addition of water (20 mL) at room temperature. The resulting mixture was extracted with EtOAc (3×30 mL). The combined organic layers were washed with brine (3×20 mL), dried over anhydrous Na₂SO₄. After filtration, the filtrate was concentrated under reduced pressure. The residue was purified by Prep-TC (EA) to afford (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one (370 mg, 83%) as a brown solid. MS ESI calculated for C₃₀H₃₂D₃F₂N₃O₂Si [M+H]⁺, 539.27 found 539.25. ¹H NMR (400 MHz, Chloroform-d) δ 8.51-8.44 (m, 1H), 7.62 (d, J=8.2 Hz, 2H), 7.46-7.28 (m, 3H), 6.83 (t, J=72.1 Hz, 1H), 6.23 (d, J=7.1 Hz, 1H), 4.95 (d, J=7.1 Hz, 1H), 3.49-3.3 8 (in, 1H), 2.86 (d, J=13.5 Hz, 1H), 1.14 (s, 21H).

### Preparation 260B: (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one

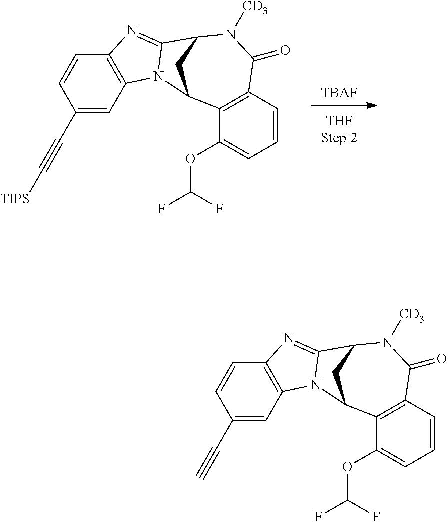

To a solution of (7R,14R)-1-(difluoromethoxy)-6-(methyl-d3)-11-((triisopropylsilyl)ethynyl)-6,7-dihydro-7,14-methanobenzo[f]benzo[4,5]imidazo[1,2-a][1,4]diazocin-5(14H)-one (370 mg, 0.687 mmol) in THF was added TBAF (824 uL, 0.824 mmol, 1M in THF) at room temperature. The resulting mixture was stirred for 1 h at room temperature. The resulting mixture was diluted with water (10 mL) and extracted with EtOAc (3×20 mL). The combined organic layers were washed with brine (3×10 mL), dried over anhydrous Na₂SO₄. After filtration, the filtrate was concentrated under reduced pressure. The residue was purified by Prep-TLC (CH₂Cl₂/MeOH 10:1) to afford (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (210 mg, 80%) as a brown solid. MS ESI calculated for C₂₁H₁₂D₃F₂N₃O₂ [M+H]⁺, 383.13 found 383.20. ¹H NMR (400 MHz, Chloroform-d) δ 8.54-8.45 (m, 1H), 7.68 (d, J=8.5 Hz, 1H), 7.63 (s, 1H), 7.48-7.39 (m, 2H), 7.35 (d, J=8.1 Hz, 1H), 7.05-6.63 (m, 1H), 6.26 (d, J=7.2 Hz, 1H), 5.06 (d, J=7.2 Hz, 1H), 3.55-3.45 (m, 1H), 3.10 (s, 1H), 2.89 (d, J=13.6 Hz, 1H).

### Example 260: N-(5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)acetamide

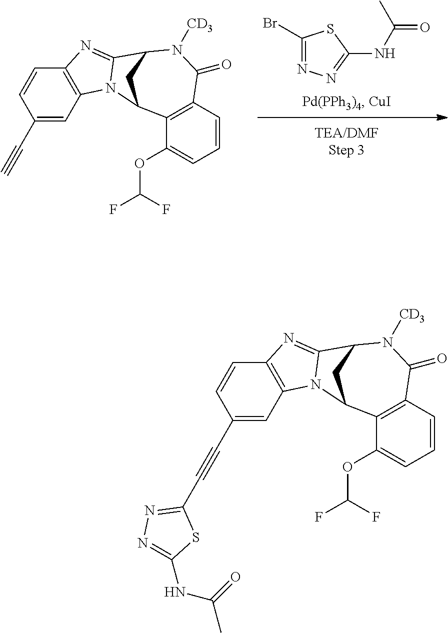

A mixture of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (50 mg, 0.131 mmol), N-(5-bromo-1,3,4-thiadiazol-2-yl)acetamide (35 mg, 0.157 mmol), CuI (2 mg, 0.013 mmol), Pd(PPh₃)₄ (9 mg, 0.013 mmol) in TEA (0.5 mL) and DMF (0.5 mL) was stirred for 16 h at 80° C. under nitrogen atmosphere. The reaction was concentrated under reduced pressure and purified by Prep-TLC (CH₂Cl₂/MeOH 10:1) followed by Prep-HPLC with the following conditions (Column: XBridge Prep Phenyl OBD Column 19*250 mm, 5 m; Mobile Phase A: Water (10 mmol/L NH₄HCO₃), Mobile Phase B: ACN; Flow rate: 60 m/min; Gradient: 21% B to 36% B in 10 min; Wave Length: 254 nm; RT1 (min): 13.211) to afford N-(5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)acetamide (9 mg, 13%) as a white solid. MS ESI calculated for C₂₅H₁₅D₃F₂N₆O₃S [M+H]⁺, 524.13 found 524.10. ¹H NMR (400 MHz, DMSO-d) S 12.70 (s, 1H), 8.35-8.24 (m, 1H), 7.85-7.61 (m, 3H), 7.55-7.41 (m, 3H), 6.29 (d, J=7.0 Hz, 1H), 5.26 (d, J=7.1 Hz, 1H), 3.63-3.48 (m, 1H), 2.84 (d, J=13.8 Hz, 1H), 2.22 (s, 3H). ¹⁹F NMR (377 MHz, DMSO-d₆) δ −81.62, −82.07, −82.30, −82.74.

### Example 261: tert-butyl (5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)carbamate

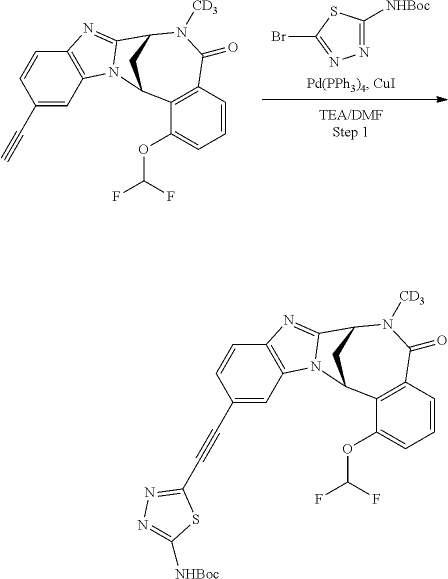

A mixture of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (100 mg, 0.262 mmol), tert-butyl N-(5-bromo-1,3,4-thiadiazol-2-yl)carbamate (293 mg, 1.048 mmol), CuI (5 mg, 0.026 mmol), Pd(PPh₃)₄ (60 mg, 0.052 mmol) in TEA (1 mL) and DMF (1 mL) was stirred for 2 h at 80° C. under nitrogen atmosphere. The resulting mixture was concentrated under reduced pressure and purified by Prep-TLC (CH₂Cl₂/MeOH 10:1) followed by Prep-HPLC with the following conditions (Column: XBridge Prep OBD C18 Column 30*150 mm, 5 m; Mobile Phase A: Water (10 mmol/L NH₄HCO₃), Mobile Phase B: ACN; Flow rate: 60 mL/min; Gradient: 36% to 54% B in 10 min; Wave Length: 254/220 nm; RT1 (min): 9.98) to afford tert-butyl (5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)carbamate (7 mg, 5%) as a white solid. MS ESI calculated for C₂₈H₂₁D₃F₂N₆O₄S [M+H]⁺, 582.17 found 582.15. ¹H NMR (400 MHz, DMSO-d₆) δ 12.31 (s, 1H), 8.31-8.19 (m, 1H), 7.76-7.53 (m, 3H), 7.50-7.34 (m, 3H), 6.22 (d, J=7.1 Hz, 1H), 5.19 (d, J=7.2 Hz, 1H), 3.54-3.42 (m, 1H), 2.77 (d, J=13.8 Hz, 1H), 1.43 (s, 9H). ¹⁹F NMR (377 MHz, DMSO-d₆) δ −81.62, −82.07, −82.29, −82.74.

### Example 262: 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolonitrile

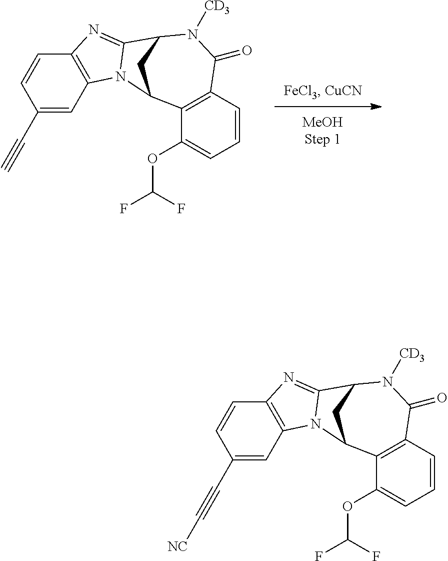

A mixture of (7R,14R)-1-(difluoromethoxy)-11-ethynyl-6-(methyl-d3)-6,7-dihydro-7,14-methanobenzo[f]benzo[4,5]imidazo[1,2-a][1,4]diazocin-5(14H)-one (160 mg, 0.418 mmol), FeCl₃ (542 mg, 3.344 mmol) and CuCN (224 mg, 2.508 mmol) in MeOH (8 mL) was stirred for overnight at 70° C. The resulting mixture was concentrated under vacuum and purified by silica gel column chromatography, eluted with DCM/MeOH (0% to 6%) followed by Prep-HPLC with the following conditions: column, C18 silica gel; mobile phase, CH₃CN in Water (10 mmol/L NH₄HCO₃), 5% to 95% gradient in 25 min; detector, 254 nm to afford 3-((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)propiolonitrile (78 mg, 45%) as a white solid. MS ESI calculated for C₂₂H₁₁D₃F₂N₄O₂[M+H]⁺, 408.13. found 408.15. ¹H NMR (400 MHz, Chloroform-d) δ 8.53-8.45 (m, 1H), 7.75-7.69 (m, 2H), 7.49-7.41 (m, 2H), 7.40-7.33 (m, 1H), 6.86 (t, J=72.6 Hz, 1H), 6.26 (d, J=7.2 Hz, 1H), 4.99 (d, J=7.1 Hz, 1H), 3.55-3.41 (m, 1H), 2.90 (d, J=13.7 Hz, 1H). ¹⁹F NMR (377 MHz, Chloroform-d) δ −80.25, −80.69, −81.00, −81.45.

### Example 263: (7R,14R)-11-((5-amino-1,3,4-thiadiazol-2-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one

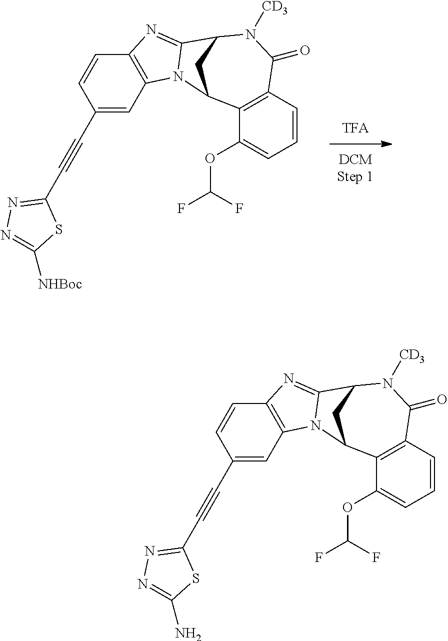 [SMILES: [2H]C([2H])([2H])N1C(=O)c2cccc(OC(F)F)c2[C@H]2C[C@@H]1c1nc3ccc(C#Cc4nnc(NC(=O)OC(C)(C)C)s4)cc3n12 ; "TFA" ; [2H]C([2H])([2H])N1C(=O)c2cccc(OC(F)F)c2[C@H]2C[C@@H]1c1nc3ccc(C#Cc4nnc(N)s4)cc3n12]

To a solution of tert-butyl (5-(((7R,14R)-1-(difluoromethoxy)-6-(methyl-d3)-5-oxo-5,6,7,14-tetrahydro-7,14-methanobenzo[f]benzo[4,5]imidazo[1,2-a][1,4]diazocin-11-yl)ethynyl)-1,3,4-thiadiazol-2-yl)carbamate (40 mg, 0.069 mmol) in DCM (0.8 mL) was TFA (0.1 mL) at room temperature. The reaction was stirred for 1 h at room temperature. The resulting mixture was concentrated under reduced pressure followed by Prep-HPLC with the following conditions (Column: YMC-Actus Triart C18 ExRS 30*150 mm, 5 μm; Mobile Phase A: Water (10 mmol/L NH₄HCO₃+0.05% NH₃H₂O), Mobile Phase B: ACN; Flow rate: 60 mL/min; Gradient: 23% B to 40% B in 10 min; Wave Length: 254/220 nm; RT1 (min): 9.93) to afford (7R,14R)-11-((5-amino-1,3,4-thiadiazol-2-yl)ethynyl)-1-(difluoromethoxy)-6-(methyl-d3)-6,7-dihydro-7,14-methanobenzo[f]benzo[4,5]imidazo[1,2-a][1,4]diazocin-5(14H)-one (18 mg, 54%) as a white solid. MS ESI calculated for C₂₃H₁₃D₃F₂N₆O₂S [M+H]⁺, 482.12. found 482.10. ¹H NMR (400 MHz, DMSO-d₆) δ 8.31-8.25 (m, 1H), 7.81-7.60 (m, 5H), 7.55-7.48 (m, 2H), 7.44-7.39 (m, 1H), 6.28 (d, J=7.1 Hz, 1H), 5.26 (d, J=7.1 Hz, 1H), 3.60-3.43 (m, 1H), 2.83 (d, J=13.8 Hz, 1H). ¹⁹F NMR (377 MHz, DMSO-d₆) δ −81.61, −82.05, −82.24, −82.69.

### II. Biological Evaluation

### TNF-α Induced HEK Blue Cellular Assay

Test articles were diluted in DMSO and serially diluted into 384 well assay plate (Corning 3765), at final concentrations ranging from 30 mM to 0.5 nM. HEK Blue™ TNFα reporter cells were added at a final density of 10,000 cell per well in assay media [DMEM (Gibco, cat #21063-029), 10% fetal bovine serum (ExcelBio, cat #FND500), 1% Penicillin-Streptomycin (Solarbio, cat #P1400-100]. TNF-α (R&D 210-TA-020/CF) was then added to the assay plate at a final concentration of 100 μg/ml. This plate was then incubated for 24 hrs at 37° C. and 5% CO₂. Secreted alkaline phosphatase expression was then measured using QUANTI-Blue™ (Invivogen), according to manufacturer instructions and read on an Envision microplate reader at 620 nm.

Inhibition data for test compound over a range of concentration was plotted as percentage inhibition of the test compound (100%=maximum inhibition). IC₅₀ values were determined after correcting for background [(sample read-mean of low control)/(mean of high control-mean of low control)] whereby the low control is DMSO without stimulation and high control is DMSO with stimulation. The IC₅₀ is defined as the concentration of test compound which produces 50% inhibition and was quantified using the 4 parameter logistic equation to fit the data.

Representative data for exemplary compounds is presented in Table 3.

TABLE 3

| Ex. No | IC₅₀ value |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | C |
| 13 | A |
| 14 | A |
| 15 | A |
| 16 | A |
| 17 | A |
| 18 | A |
| 19 | A |
| 20 | A |
| 21 | A |
| 22 | A |
| 23 | B |
| 24 | A |
| 25 | C |
| 26 | B |
| 27 | A |
| 28 | A |
| 29 | A |
| 30 | A |
| 31 | A |
| 32 | A |
| 33 | D |
| 34 | A |
| 35 | D |
| 36 | A |
| 37 | A |
| 38 | A |
| 39 | A |
| 40 | A |
| 41 | A |
| 42 | B |
| 43 | A |
| 44 | A |
| 45 | A |
| 46 | B |
| 47 | D |
| 48 | A |
| 49 | B |
| 50 | A |
| 51 | A |
| 52 | A |
| 53 | A |
| 54 | D |
| 55 | D |
| 56 | A |
| 57 | B |
| 58 | D |
| 59 | D |
| 60 | A |
| 61 | B |
| 62 | A |
| 63 | A |
| 64 | D |
| 65 | D |
| 66 | B |
| 67 | B |
| 68 | A |
| 69 | B |
| 70 | D |
| 71 | B |
| 72 | A |
| 73 | A |
| 74 | A |
| 75 | B |
| 76 | A |
| 77 | A |
| 78 | B |
| 79 | B |
| 80 | A |
| 81 | B |
| 82 | D |
| 83 | A |
| 84 | A |
| 85 | A |
| 86 | A |
| 87 | A |
| 88 | A |
| 89 | B |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | D |
| 105 | A |
| 106 | A |
| 107 | A |
| 108 | A |
| 109 | B |
| 110 | A |
| 111 | A |
| 112 | A |
| 113 | A |
| 114 | A |
| 115 | A |
| 116 | C |
| 117 | B |
| 118 | A |
| 119 | A |
| 120 | A |
| 121 | A |
| 123 | B |
| 124 | A |
| 125 | A |
| 126 | A |
| 127 | A |
| 128 | A |
| 129 | A |
| 130 | B |
| 131 | A |
| 132 | A |
| 133 | B |
| 134 | A |
| 135 | A |
| 136 | A |
| 137 | A |
| 138 | D |
| 139 | A |
| 140 | A |
| 141 | A |
| 142 | A |
| 143 | A |
| 144 | B |
| 145 | A |
| 146 | B |
| 147 | A |
| 148 | A |
| 149 | A |
| 150 | A |
| 151 | B |
| 152 | B |
| 153 | B |
| 154 | A |
| 155 | A |
| 156 | B |
| 157 | B |
| 158 | A |
| 159 | A |
| 160 | A |
| 161 | A |
| 162 | A |
| 163 | B |
| 164 | B |
| 165 | B |
| 166 | A |
| 167 | A |
| 168 | B |
| 169 | B |
| 170 | A |
| 171 | A |
| 172 | A |
| 173 | A |
| 174 | A |
| 175 | A |
| 176 | B |
| 177 | A |
| 178 | B |
| 179 | D |
| 180 | D |
| 181 | D |
| 182 | B |
| 183 | A |
| 184 | A |
| 185 | A |
| 186 | A |
| 187 | A |
| 188 | B |
| 189 | D |
| 190 | B |
| 191 | D |
| 192 | D |
| 193 | B |
| 194 | D |
| 195 | A |
| 196 | B |
| 197 | D |
| 198 | D |
| 199 | B |
| 200 | B |
| 201 | D |
| 202 | B |
| 203 | A |
| 204 | A |
| 205 | A |
| 206 | A |
| 207 | A |
| 208 | D |
| 209 | D |
| 210 | B |
| 211 | A |
| 212 | B |
| 213 | D |
| 214 | B |
| 215 | D |
| 216 | D |
| 217 | D |
| 218 | D |
| 219 | A |
| 220 | A |
| 221 | B |
| 222 | A |
| 223 | B |
| 224 | A |
| 225 | A |
| 226 | A |
| 227 | A |
| 228 | A |
| 229 | B |
| 230 | A |
| 231 | A |
| 232 | B |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | B |
| 249 | A |
| 250 | A |
| 251 | D |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| — | ″ |
| — | ″ |

Note:
IC₅₀ data are designated within the following ranges:
A: ≤0.1 μM
B: >0.1 μM to ≤1.0 μM
C: >1 μM to ≤10 μM
D: >10 μM to ≤30 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1. A compound of Formula (I), or pharmaceutically acceptable salt, solvate, N-oxide, or deuteroisotope thereof: 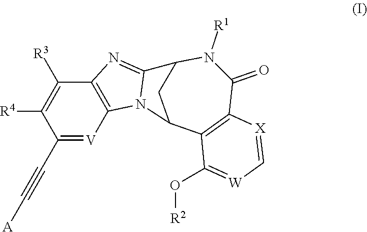
wherein,
Group A is selected from H, —CO₂R⁶, —CON(R⁶)₂, optionally substituted C1-C6 alkyl, optionally substituted C1-C6 alkyl further substituted with —O-(optionally substituted heterocyclyl), optionally substituted C3-C6 carbocyclyl, optionally substituted C4-C7 carbocyclylalkyl, optionally substituted heterocyclyl, optionally substituted heterocyclyl further substituted with an optionally substituted carbocyclyl or heterocyclyl, optionally substituted heterocyclylalkyl, optionally substituted heteroaryl, optionally substituted heteroaralkyl, optionally substituted aryl, optionally substituted aralkyl;
V is N or C—R⁵;
W is N or C—H;
X is N or C—H;
R¹ is selected from hydrogen, optionally substituted C1-C6 alkyl, optionally substituted C3-C6 cycloalkyl, or optionally substituted C4-C7 cycloalkylalkyl;
R² is hydrogen, or optionally substituted C1-C3 alkyl;
R³ is hydrogen or halogen;
R⁴ is hydrogen, halogen, or optionally substituted C1-C6 alkyl;
R⁵ is hydrogen, halogen, or optionally substituted C1-C6 alkyl; and
R⁶ is hydrogen or optionally substituted C1-C6 alkyl.

2. (canceled)

3. The compound of claim 1, or pharmaceutically acceptable salt, solvate, N-oxide, or deuteroisotope thereof, wherein R¹ is CH₃ or CD₃.

4. The compound of claim 1, or pharmaceutically acceptable salt, solvate, N-oxide, or deuteroisotope thereof, wherein R¹ is optionally substituted C1-C6 alkyl.

5. (canceled)

6. (canceled)

7. The compound of claim 1, or pharmaceutically acceptable salt, solvate, N-oxide, or deuteroisotope thereof, wherein R² is —CHF₂.

8. The compound of claim 1, or pharmaceutically acceptable salt, solvate, N-oxide, or deuteroisotope thereof, wherein R⁴ is hydrogen or halogen.

9. (canceled)

10. The compound of claim 1, or pharmaceutically acceptable salt, solvate, N-oxide, or deuteroisotope thereof, wherein R³ is hydrogen.

11. The compound of claim 1, or pharmaceutically acceptable salt, solvate, N-oxide, or deuteroisotope thereof, wherein V is N.

12. (canceled)

13. The compound of claim 1, or pharmaceutically acceptable salt, solvate, N-oxide, or deuteroisotope thereof, wherein V is C—R¹¹ and R¹¹ is hydrogen.

14. The compound of claim 1, or pharmaceutically acceptable salt, solvate, N-oxide, or deuteroisotope thereof, wherein W is C—H and X is C—H.

15. (canceled)

16. (canceled)

17. (canceled)

18. The compound of claim 1, or pharmaceutically acceptable salt, solvate, N-oxide, or deuteroisotope thereof, wherein Group A is optionally substituted C1-C6 alkyl.

19. The compound of claim 1, or pharmaceutically acceptable salt, solvate, N-oxide, or deuteroisotope thereof, wherein Group A is optionally substituted C1-C6 alkyl further substituted with —O-(optionally substituted heterocyclyl).

20. The compound, or pharmaceutically acceptable salt, solvate, N-oxide, or deuteroisotope thereof, of claim 19, wherein optionally substituted C1-C6 alkyl further substituted with —O-(optionally substituted heterocyclyl) is substituted with an —O—(N-methylpiperidine).

21. The compound, or pharmaceutically acceptable salt, solvate, N-oxide, or deuteroisotope thereof, of claim 18, wherein the optionally substituted C1-C6 alkyl is substituted with at least an amino group or a hydroxy group.

22. The compound of claim 1, or pharmaceutically acceptable salt, solvate, N-oxide, or deuteroisotope thereof, wherein Group A is optionally substituted C3-C6 carbocyclyl.

23. The compound, or pharmaceutically acceptable salt, solvate, N-oxide, or deuteroisotope thereof, of claim 22, wherein the optionally substituted C3-C6 carbocyclyl is substituted with at least an amino group or a hydroxy group.

24. The compound of claim 1, or pharmaceutically acceptable salt, solvate, N-oxide, or deuteroisotope thereof, wherein Group A is optionally substituted 5- or 6-membered heterocyclyl.

25. The compound, or pharmaceutically acceptable salt, solvate, N-oxide, or deuteroisotope thereof, of claim 24, wherein the optionally substituted 5- or 6-membered heterocyclyl is an optionally substituted tetrahydrofuran, optionally substituted tetrahydropyran, optionally substituted piperidine, optionally substituted morpholine, optionally substituted piperazine, or optionally substituted pyrrolidine.

26. (canceled)

27. The compound of claim 1, or pharmaceutically acceptable salt, solvate, N-oxide, or deuteroisotope thereof, wherein Group A is optionally substituted 5- or 6-membered heteroaryl.

28. The compound of claim 27, or pharmaceutically acceptable salt, solvate, N-oxide, or deuteroisotope thereof, wherein the optionally substituted 5- or 6-membered heteroaryl is selected from optionally substituted pyrazole, optionally substituted thiazole, optionally substituted oxazole, optionally substituted imidazole, optionally substituted triazole, optionally substituted pyridine or optionally substituted pyrimidine.

29. (canceled)

30. (canceled)

31. The compound of claim 1, or pharmaceutically acceptable salt, solvate, N-oxide, or deuteroisotope thereof, wherein Group A is optionally substituted aryl.

32. The compound, or pharmaceutically acceptable salt, solvate, N-oxide, or deuteroisotope thereof, of claim 31, wherein the optionally substituted aryl is optionally substituted phenyl.

33. The compound of claim 1, or pharmaceutically acceptable salt, solvate, N-oxide, or deuteroisotope thereof, wherein Group A is optionally substituted C4-C7 carbocyclylalkyl.

34. The compound, or pharmaceutically acceptable salt, solvate, N-oxide, or deuteroisotope thereof, of claim 33, wherein the optionally substituted C4-C7 carbocyclylalkyl is —CH₂-(optionally substituted carbocyclyl).

35. The compound of claim 1, or pharmaceutically acceptable salt, solvate, N-oxide, or deuteroisotope thereof, wherein Group A is optionally substituted heterocyclyl further substituted with an optionally substituted heterocyclyl.

36. The compound, or pharmaceutically acceptable salt, solvate, N-oxide, or deuteroisotope thereof, of claim 35, wherein the optionally substituted heterocyclyl further substituted with an optionally substituted carbocyclyl or heterocyclyl is selected from:

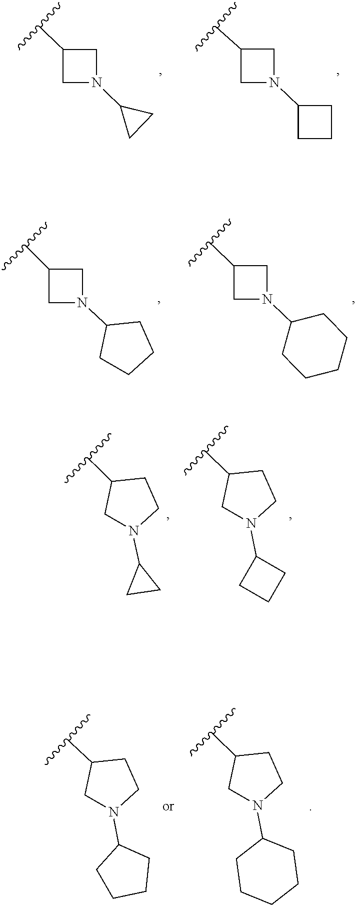

37. The compound, or pharmaceutically acceptable salt, solvate, N-oxide, or deuteroisotope thereof, of claim 35, wherein the optionally substituted heterocyclyl further substituted with an optionally substituted carbocyclyl or heterocyclyl is selected from:

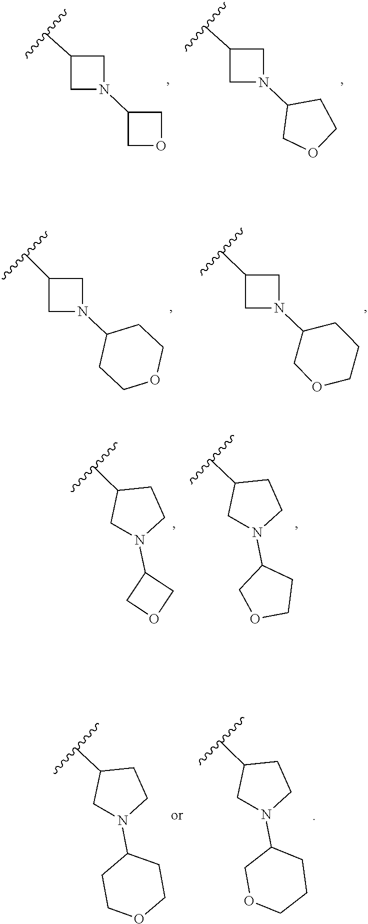

38. A compound described in Table 1, or pharmaceutically acceptable salt, solvate, N-oxide, or deuteroisotope thereof.

39. A compound described in Table 2, or pharmaceutically acceptable salt, solvate, N-oxide, or deuteroisotope thereof.

40. A pharmaceutical composition comprising the compound, or pharmaceutically acceptable salt or solvate or N-oxide thereof, of claim 1 and a pharmaceutically acceptable excipient or carrier.

41. (canceled)

42. (canceled)

43. (canceled)

44. (canceled)

45. (canceled)

46. A method of treating an inflammatory or autoimmune disease or disorder in a patient in need thereof, comprising administering to the patient a therapeutically effectively amount of the compound of claim 1, or pharmaceutically acceptable salt or solvate or N-oxide thereof.

47. (canceled)

48. (canceled)

49. (canceled)

50. (canceled)

51. (canceled)

52. (canceled)

53. The method of claim 46, wherein the disease or disorder is selected from rheumatoid arthritis, psoriatic arthritis, systemic onset juvenile idiopathic arthritis, multiple sclerosis, lupus nephritis, systemic lupus erythematosus, psoriasis, Crohn's disease, colitis, asthma, graft versus host disease, allograft rejection, chronic obstructive pulmonary disease, Alzheimer's disease, Graves' disease, cutaneous lupus, ankylosing spondylitis, cryopyrin-associated periodic syndromes (CAPS), gout, gouty arthritis, TNF receptor associated periodic syndrome (TRAPS), Wegener's granulomatosis, sarcoidosis, familial Mediterranean fever (FMF), neuropathic pain, adult onset stills, Inflammatory Bowel Diseases (IBD), ulcerative colitis, spondyloarthritis, Behçet's Disease, Palmoplantar Pustulosis (PPP), Atopic Dermatitis, Sepsis, Fibromyalgia, Schnitzler Syndrome, Blau Syndrome, Pyogenic Arthritis, Pyoderma Gangrenosum, and chronic Recurrent Multifocal Osteomyelitis (CRMO).
